SEQUENCE LISTING (18 sequences)

SEQ ID NO: 1 - protein, 1409 aa, synthetic construct
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 2 - protein, 1367 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 4 - protein, 1300 aa, synthetic construct
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 5 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 6 - protein, 1129 aa, synthetic construct
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 7 - protein, 11 aa, synthetic construct
YGRKKRRQRRR

SEQ ID NO: 8 - protein, 12 aa, synthetic construct
RRQRRTSKLMKR

SEQ ID NO: 9 - protein, 27 aa, synthetic construct
GWTLNSAGYLLGKINLKALAALAKKIL

SEQ ID NO: 10 - protein, 33 aa, synthetic construct
KALAWEAKLAKALAKALAKHLAKALAKALKCEA

SEQ ID NO: 11 - protein, 16 aa, synthetic construct
RQIKIWFQNRRMKWKK

SEQ ID NO: 12 - protein, 11 aa, synthetic construct
YGRKKRRQRRR

SEQ ID NO: 13 - protein, 9 aa, synthetic construct
RKKRRQRRR

SEQ ID NO: 14 - protein, 11 aa, synthetic construct
YGRKKRRQRRR

SEQ ID NO: 15 - protein, 8 aa, synthetic construct
RKKRRQRR

SEQ ID NO: 16 - protein, 11 aa, synthetic construct
YARAAARQARA

SEQ ID NO: 17 - protein, 11 aa, synthetic construct
THRLPRRRRRR

SEQ ID NO: 18 - protein, 11 aa, synthetic construct
GGRRARRRRRR

(19) United States
(12) Patent Application Publication
(10) Pub. No.: US 2026/0242829 A1
(43) Pub. Date: Aug. 20, 2026

(54) Use of Inhibitors to Increase Efficiency of CRISPR/Cas Insertions

(71) Applicant: ASTRAZENECA AB, Södertälje (SE)

(72) Inventors: Marcello MARESCA, Södertälje (SE); Sasa SVIKOVIC, Södertälje (SE); Nina AKRAP, Södertälje (SE); Sandra WIMBERGER, Södertälje (SE)

(21) Appl. No.: 19/469,699

(22) PCT Filed: Mar. 28, 2024
(86) PCT No.: PCT/IB2024/053026

§ 371 Date: Sep. 26, 2025

Related U.S. Application Data
(60) Provisional application No. 63/492,847, filed on Mar. 29, 2023.

(51) Int. Cl. C12N 15/90; C07D 487/04; C07D 519/00; C07K 14/705; C12N 5/074; C12N 5/0783; C12N 9/22; C12N 9/99; C12N 15/11; C12N 15/86; C12N 15/88

(52) U.S. Cl. CPC C12N 15/907; C07D 487/04; C07D 519/00; C07K 14/7051; C12N 5/0636; C12N 5/0696; C12N 9/226; C12N 9/99; C12N 15/111; C12N 15/86; C12N 15/88; C12N 2310/20; C12N 2510/00; C12N 2710/10043; C12N 2740/10043; C12N 2740/15043; C12N 2750/14143

(57) ABSTRACT

The present disclosure provides methods of inserting a polynucleotide of interest into the genome of a eukaryotic cell, wherein said methods comprise improving the efficiency of CRISPR/Cas-mediated polynucleotide insertion by addition of an inhibitor of the microhomology—mediated end-joining (MMEJ) pathway to the eukaryotic cell. The present disclosure further provides compositions for inserting a polynucleotide of interest into the genome of a eukaryotic cell, and kits for inserting a gene of interest into the genome of a eukaryotic cell.

### RELATED APPLICATIONS

This application claims priority to U.S. Provisional Application 63/492,847, filed Mar. 29, 2023, which is incorporated by reference herein in its entirety for all purposes.

### BACKGROUND

The development of cost-efficient and reliable methods for precise targeted alterations to the genome of living cells has been a long-standing goal. Genome editing has the potential to eliminate genes responsible for a particular disorder (i.e. a gene “knock-out”), or alternatively, provide a means for gene manipulation or insertion to correct a genetic deficiency or enhance a biological process via a gene “knock-in.” Genome editing can be applied for treatment of a multitude of disorders, including treatment of inherited disorders, hematological disorders and cancer, and in methods of immunotherapy.

Clustered Regularly Interspaced Short Palindromic Repeats (CRISPR) and CRISPR-associated (Cas) systems are prokaryotic immune systems first discovered by Ishino in E. coli (Ishino et al., Journal of Bacteriology 169(12):5429-5433 (1987)). The prokaryotic immune system provides immunity against viruses and plasmids by targeting the nucleic acids of the viruses and plasmids in a sequence-specific manner. See also Soret et al., Nature Reviews Microbiology 6(3):181-186 (2008).

Since its original discovery, multiple groups have performed extensive research around potential applications of the CRISPR system in genetic engineering, including gene editing (Jinek et al., Science 337(6096):816-821 (2012); Cong et al., Science 339(6121):819-823 (2013); and Mali et al., Science 339(6121):823-826 (2013)). The CRISPR-Cas9 gene editing system has been used successfully in a wide range of organisms and cell lines. In addition to genome editing, the CRISPR system has a multitude of other applications, including regulating gene expression, genetic circuit construction, and functional genomics, amongst others (reviewed in Sander et al., Nature Biotechnology 32:347-355 (2014)).

The Cas9 endonuclease generates a double-stranded DNA break at the target sequence, upstream of a protospacer adjacent motif (PAM). The target sequence can then be removed, or a sequence of interest can be inserted into the target sequence using an endogenous repair pathway of the cell. Endogenous DNA repair pathways include the Non-Homologous End Joining (NHEJ) pathway, Microhomology-Mediated End Joining (MMEJ) pathway, and the Homology Directed Repair (HDR) pathway. NHEJ, MMEJ, and HDR pathways repair double-stranded DNA breaks, but repair of such double-stranded DNA breaks may result in insertions or deletions at the double-stranded break site. In NHEJ, a homologous template is not required for repairing breaks in the DNA. NHEJ repair can be error-prone, although errors are decreased when the DNA break includes compatible overhangs. NHEJ and MMEJ are mechanistically distinct DNA repair pathways with different subsets of DNA repair enzymes involved in each of them. Unlike NHEJ, which can be precise in some cases, or error-prone in some cases, MMEJ is always error-prone and results in both deletion and insertions at the site under repair. MMEJ-associated deletions are due to the micro-homologies (2-10 base pairs) at both sides of a double-strand break. In contrast, HDR requires a homologous template to direct repair, but HDR repairs are typically high-fidelity and less error-prone. HDR-driven repair of double-stranded DNA breaks is therefore preferable to NHEJ- or MMEJ-mediated repair; however, in many cell types HDR is limited by the activity of NHEJ at all cell cycle stages, and HDR is primarily utilized in the S phase of cell growth (Mao et al., Cell Cycle, 7:2902-2906 (2008)).

### SUMMARY

In some embodiments, the present disclosure relates to methods of increasing the efficiency of CRISPR/Cas-mediated gene insertion. In some embodiments, the method comprises inserting a polynucleotide of interest into the genome of a eukaryotic cell, the method comprising (a) adding an inhibitor of the MMEJ pathway to a composition comprising the eukaryotic cell, (b) adding a Cas effector protein to the composition, and (c) adding the polynucleotide of interest to the composition, wherein the polynucleotide of interest is inserted into the genome of the eukaryotic cell by homology directed repair (HDR) or single-stranded template repair (SSTR).

In some embodiments, step (a) of the method further comprises adding an inhibitor of the non-homologous end-joining (NHEJ) pathway.

In some embodiments, the method further comprises (d) adding a polynucleotide comprising an RNA guide sequence, a Cas-binding region, a DNA template sequence, or combinations thereof to the composition.

In some embodiments, the Cas effector protein and the polynucleotide of (d) are added in the form of a ribonucleoprotein (RNP).

In some embodiments, the Cas effector protein is added in (b) by adding a Cas polynucleotide encoding the Cas effector protein.

In some embodiments, the polynucleotide of interest, the polynucleotide of step (d) and the Cas polynucleotide are encoded on a single vector. In some embodiments, the polynucleotide of interest is added as DNA. In some embodiments, the polynucleotide of step (d) is added as DNA. In some embodiments, the polynucleotide of step (d) is added as RNA. In some embodiments, the Cas effector polynucleotide is added as DNA. In some embodiments, the Cas polynucleotide is added as RNA. In some embodiments, the Cas polynucleotide is added as mRNA.

In some embodiments, the vector is a viral vector. In some embodiments, the viral vector is a retrovirus, a lentivirus, an adenovirus, or an adeno-associated virus (AAV).

In some embodiments, the Cas effector protein, the polynucleotide of interest, and the polynucleotide of (d) are added to the eukaryotic cell by microinjection, electroporation, or via a lipid nanoparticle, liposome, exosome, gold nanoparticle or a DNA nanoclew.

In some embodiments, the vector is added to the composition comprising the eukaryotic cell by transfecting the eukaryotic cell.

In some embodiments, the Cas effector protein is a Cas9 nuclease, a Cas12a nuclease, or a Cas12f nuclease. In some embodiments, the Cas effector protein is a Cas9 nuclease. In some embodiments, the Cas9 nuclease is a Cas9 nuclease fused to a reverse transcriptase, a Cas9 nuclease fused to a DNA polymerase, a Cas9 nuclease fused to DNJS, a Cas9 nickase, a Cas9 fused to a Geminin degron domain, or a Cas9 nuclease fused to CTIP.

In some embodiments, the polynucleotide of interest is added via a vector. In some embodiments, the vector is a viral vector. In some embodiments, the viral vector is a retrovirus, a lentivirus, an adenovirus, or an adeno-associated virus (AAV).

In some embodiments, the polynucleotide of interest comprises a gene of interest. In some embodiments, the polynucleotide of interest is 1 to 50 base pairs in length. In some embodiments, the polynucleotide of interest is 1 to 10 base pairs in length. In some embodiments, the polynucleotide of interest is 50 to 5000 base pairs in length.

In some embodiments, the polynucleotide of interest is single-stranded. In some embodiments, the polynucleotide of interest is double stranded. In some embodiments, the polynucleotide of interest is a hybrid polynucleotide comprising single-stranded and double-stranded regions. In some embodiments, the hybrid polynucleotide comprises double-stranded sequences at the 5′ and 3′ ends and an internal single-stranded sequence. In some embodiments, the polynucleotide of interest is double-stranded with blunt ends. In some embodiments, the polynucleotide of interest is double-stranded with a 3′ overhang. In some embodiments, the polynucleotide of interest is double-stranded with a 5′ overhang. In some embodiments, the polynucleotide of interest is a circular polynucleotide.

In some embodiments, the polynucleotide of interest comprises a chemical modification which enhances the activity, distribution, or uptake of the polynucleotide.

In some embodiments, the inhibitor of the MMEJ pathway is an inhibitor of POL Q/DNA polymerase q. In some embodiments, the inhibitor of POL Q is a compound of formula (I):

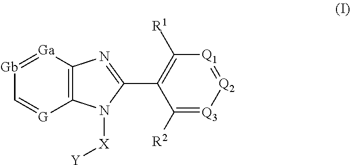

or any stereoisomer thereof or pharmaceutically acceptable salt thereof;
wherein,
R¹ and R² are each, independently, H, halo, C₁-C₃ alkyl, C₁-C₃ alkoxy, C₁-C₃ haloalkyl, C₁-C₃ hydroxyalkyl, —CN, C₂-C₄ alkyne, or C₂-C₆ alkoxyalkyl;
Q¹, Q², and Q³ are, independently N, C-L-R, or CR^{x}, wherein no more than one of Q¹, Q², and Q³ is C-L-R;
L is a bond, —O—; —C(O)—; —O(CH₂)ₚC(O)—; —C(O)NR^{y}—; —O(CH₂)ₚC(O)NR^{y}—; —O(CH₂)ₚNR^{y}; —NR^{y}—; —(CH₂)ₚ—; —(CH₂)ₚNR^{y}—; —(CH₂)ₚO—; —(CH₂)ₚC(O)—; —(CH₂)ₚC(O)O—; —O(CH₂)ₚ—;
p is, independently, 1, 2, or 3
R is H, R^{a}, R^{b}, R^{c}, or R^{d};
R^{a} is a 3-10 membered heterocycle optionally substituted with 1 to 4 substituents selected from amino, carboxy, halo, hydroxy, oxo, —CN, —S(O)₂OH, C1-C4 alkylamino, C1-C5 alkoxy, C₂-C₅ alkoxyalkyl, 4-6 membered heterocycle, and C₁-C₇ alkyl, wherein the C1-C7 alkyl is optionally substituted with 1 to 4 substituents selected from amino, carboxy, halo, hydroxy, oxo, —CN, C₂-C₈ ester, and C₁-C₅ alkoxy;
   R^{b} is a C₁-C₇ alkyl, wherein one or two methylene groups from the C₁-C₇ alkyl are optionally replaced with NR^{e} or O and one or two single bonds in a C₂-C₇ alkyl chain are optionally replaced with a double or triple bond(s), wherein the C₁-C₇ alkyl is optionally substituted with 1 to 4 substituents selected from: halo, oxo, hydroxy, carboxyl, amino, —CN, C₂-C₄ alkynyl, C₂-C₆ carbamate, C₁-C₈ amide, C₁-C₄ sulfonyl, C₁-C₄ sulfonamide, C₁-C₄ alkylamino, C₁-C₅ alkoxy, C₃-C₆ carbocycle, and 3-10 membered heterocycle,
wherein the C₃-C₆ carbocycle is optionally substituted with 1 to 4 substituents selected from hydroxy, halo, and carboxy;
wherein the 3-10 membered heterocycle is optionally substituted with 1 to 4 substituents selected from amino, carboxy, halo, hydroxy, oxo, —CN, —S(O)₂OH, C₁-C₄ alkylamino, C₁-C₅ alkoxy, C₂-C₅ alkoxyalkyl, 4-6 membered heterocycle, and C₁-C₇ alkyl, wherein the C₁-C₇ alkyl is optionally substituted with 1 to 4 substituents selected from amino, carboxy, halo, hydroxy, oxo, —CN, C₂-C₈ ester, and C₁-C₅ alkoxy;
R^{c} is a C₃-C₆ carbocycle optionally substituted with 1 to 4 substituents selected from hydroxy halo, and carboxy;
R^{d} is C₁-C₄ sulfonyl or C₁-C₄ sulfonamide;
R^{y} is H, C₁-C₃ alkyl, or C₁₋₃ haloalkyl;
R^{x} is H, halo, hydroxy, —CN, —NH₂, C₁-C₃ alkoxy, C₁-C₃ alkyl, or C₁₋₃ haloalkyl;
R^{c} is H, halo, C₁-C₈ alkyl, or C₁-C₈ haloalkyl;
X is a C₁-C₄ alkylene;
Y is a phenyl or 5-6 membered heteroaryl, wherein the phenyl or heteroaryl is optionally substituted with 1-3 substituents selected from halo, C₁-C₃ alkyl, C₁-C₃ alkoxy, —CN, C₁-C₃ haloalkyl, and cyclopropyl;
G is N or CH;
Gₐ and G_{b} are N, CH, or CR⁵ wherein one, and only one, of Gₐ and G_{b} is N or CH and one, and only one, of Gₐ and G_{b} is CR⁵;
R⁵ is;

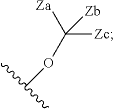

Zₐ and Z_{b} are, independently, C₁-C₃ alkyl or C₁-C₃ haloalkyl, or Zₐ and Z_{b} form a 3-6 membered carbocycle or heterocycle; and
Z_{c} is H, —CN, C₁-C₃ alkyl, C₁-C₃ haloalkyl, or C₂-C₄ alkyne,
or combinations thereof.

In some embodiments the inhibitor of POL Q is a compound disclosed herein, or combinations thereof.

In some embodiments, the inhibitor of POL Q is a compound listed in Table I, Table II, Table III, a pharmaceutically acceptable salt thereof, or combinations thereof.

In some embodiments, the inhibitor of POL Q is 9-Benzyl-8-(2-chloro-4-(2-(4-methylpiperazin-1-yl)ethoxy)phenyl)-6-(1-methylcyclopropoxy)-9H-purine (Compound 1), or a salt thereof. In some embodiments, the inhibitor of POL Q is 9-Benzyl-8-(2-chloro-4-(2-(4-methylpiperazin-1-yl)ethoxy)phenyl)-6-(1-methylcyclopropoxy)-9H-purine (Compound 1)

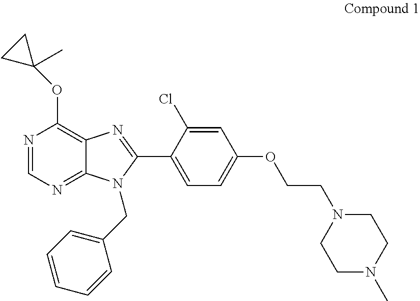 [SMILES: CN1CCN(CCOc2ccc(-c3nc4c(OC5(C)CC5)ncnc4n3Cc3ccccc3)c(Cl)c2)CC1]

In some embodiments, the inhibitor of the MMEJ pathway in the composition comprising the eukaryotic cell is about 0.01 mM to about 1 mM, about 0.1 mM to about 1 mM, about 0.1 mM to about 0.5 mM, about 0.1 mM to about 100 mM, or about 1 mM to about 50 mM.

In some embodiments, the inhibitor of the NHEJ pathway is an inhibitor of DNA-dependent protein kinase (DNA-PK). In some embodiments, the inhibitor of DNA-PK is M3814, M9831/VX984, Nu7441, KU0060648, AZD7648, or combinations thereof. In some embodiments, the inhibitor of DNA-PK is AZD7648. In some embodiments, the inhibitor of DNA-PK is a peptide.

In some embodiments, the inhibitor of the NHEJ pathway in the composition comprising the eukaryotic cell is about 0.01 mM to about 1 mM, about 0.1 mM to about 1 mM, about 0.1 mM to about 0.5 mM, about 0.1 mM to about 100 mM, or about 1 mM to about 50 mM.

In some embodiments, the inhibitor of the MMEJ pathway is added to the composition comprising the eukaryotic cell 0 minutes to about 48 hours, 0 minutes to about 24 hours, 0 minutes to about 12 hours, 0 minutes to about 6 hours, or 0 minutes to about 1 hour before the Cas effector protein is added to the composition. In some embodiments, the inhibitor of the MMEJ pathway is added to the composition comprising the eukaryotic cell 0 minutes to about 1 hour after the Cas effector protein is added to the composition comprising the eukaryotic cell.

In some embodiments, the inhibitor of the NHEJ pathway is added to the composition comprising the eukaryotic cell 0 minutes to about 48 hours, 0 minutes to about 24 hours, 0 minutes to about 12 hours, 0 minutes to about 6 hours, or 0 minutes to about 1 hour before the Cas effector protein is added to the composition. In some embodiments, the inhibitor of the NHEJ pathway is added to the composition comprising the eukaryotic cell 0 minutes to about 1 hour after the Cas effector protein is added to the composition comprising the eukaryotic cell.

In some embodiments, the inhibitor of the MMEJ pathway and the inhibitor of the NHEJ pathway are added to the composition comprising the eukaryotic cell at the same time. In some embodiments, the inhibitor of the MMEJ pathway and the inhibitor of the NHEJ pathway are added to the composition comprising the eukaryotic cell at different times.

In some embodiments, the inhibitor of the MMEJ pathway, the inhibitor of the NHEJ pathway, and the Cas effector protein are added to the composition comprising the eukaryotic cell at the same time.

In some embodiments, the inhibitor of the MMEJ pathway is in the composition comprising the eukaryotic cell for about 1 to about 300 hours, for about 10 to about 100 hours, or about 20 to about 80 hours.

In some embodiments, the inhibitor of the MMEJ pathway is added to the composition comprising the eukaryotic cell at least once, at least twice, or at least three times.

In some embodiments, the inhibitor of the NHEJ pathway is in the composition comprising the eukaryotic cell for about 1 to about 300 hours, for about 10 to about 100 hours, or about 20 to about 80 hours.

In some embodiments, the inhibitor of the NHEJ pathway is added to the composition comprising the eukaryotic cell at least once, at least twice, or at least three times.

In some embodiments, the composition comprising the eukaryotic cell is a cell culture. In some embodiments, the cell culture is an in vitro cell culture or an ex vivo cell culture. In some embodiments, the eukaryotic cell is in vivo.

In some embodiments, the cell culture comprises a cell extract.

In some embodiments, the eukaryotic cell is a lymphocyte. In some embodiments, the lymphocyte comprises a chimeric antigen receptor (CAR) or a T cell receptor (TCR).

In some embodiments, the eukaryotic cell is a pluripotent stem cell. In some embodiments, the pluripotent stem cell is an induced pluripotent stem cell (iPSC).

In some embodiments, the cell culture is a mammalian cell culture.

In some embodiments, the present disclosure relates to methods of increasing the efficiency of CRISPR/Cas-mediated gene insertion comprising inserting a polynucleotide of interest into a genome of a eukaryotic cell comprising a genomically-integrated Cas polynucleotide. In some embodiments, the disclosure provides a method of inserting a polynucleotide of interest into a genome of a eukaryotic cell, the method comprising: (a) adding an inhibitor of the microhomology-mediated end joining (MMEJ) pathway to a composition comprising the eukaryotic cell, and (b) adding the polynucleotide of interest to the composition, wherein the genome comprises a genomically integrated Cas polynucleotide, and wherein the polynucleotide of interest is inserted into the genome by homology directed repair (HDR) or single-stranded template repair (SSTR). In some embodiments, the genomically-integrated Cas polynucleotide is inducible.

In some embodiments, the method further comprises adding an inhibitor of the non-homologous end joining (NHEJ) pathway to the composition.

In some embodiments, the method further comprises (c) adding a polynucleotide comprising an RNA guide sequence, a Cas-binding region, a DNA template sequence, or combinations thereof, to the composition.

In some embodiments, (i) the polynucleotide of interest and (ii) the polynucleotide of (c) are encoded on a vector. In some embodiments, the polynucleotide of interest is added as DNA.

In some embodiments, the polynucleotide of (c) is added as DNA. In some embodiments, the polynucleotide of (c) is added as RNA.

In some embodiments, the vector is a viral vector. In some embodiments, the viral vector is a retrovirus, a lentivirus, an adenovirus, or an adeno-associated virus (AAV). In some embodiments, the vector is added to the composition comprising the eukaryotic cell by transfecting the eukaryotic cell.

In some embodiments, the Cas effector protein is a Cas9 nuclease, a Cas12a nuclease, or a Cas12f nuclease. In some embodiments, the Cas effector protein is a Cas9 nuclease. In some embodiments, the Cas9 nuclease is a Cas9 nuclease fused to a reverse transcriptase, a Cas9 nuclease fused to a DNA polymerase, a Cas9 nuclease fused to DNlS, a Cas9 nickase, a Cas9 fused to a Geminin degron domain, or a Cas9 nuclease fused to CTIP.

In some embodiments, the polynucleotide of interest is added via a vector. In some embodiments, the vector is a viral vector. In some embodiments, the viral vector is a retrovirus, a lentivirus, an adenovirus, or an adeno-associated virus (AAV).

In some embodiments, the polynucleotide of interest comprises a gene of interest. In some embodiments, the polynucleotide of interest is 1 to 50 base pairs in length, 1 to 10 base pairs in length, or 50 to 5000 base pairs in length.

In some embodiments, the polynucleotide of interest is single-stranded. In some embodiments, the polynucleotide of interest is double stranded. In some embodiments, the polynucleotide of interest is a hybrid polynucleotide comprising single-stranded and double-stranded regions. In some embodiments, the hybrid polynucleotide comprises double-stranded sequences at the 5′ and 3′ ends and an internal single-stranded sequence. In some embodiments, the polynucleotide of interest is double-stranded with blunt ends. In some embodiments, the polynucleotide of interest is double-stranded with a 3′ overhang. In some embodiments, the polynucleotide of interest is double-stranded with a 5′ overhang. In some embodiments, the polynucleotide of interest is a circular polynucleotide.

In some embodiments, the polynucleotide comprises a chemical modification which enhances the activity, distribution, or uptake of the polynucleotide.

In some embodiments, the inhibitor of the MMEJ pathway is an inhibitor of POL Q/DNA polymerase q. In some embodiments, the inhibitor of POL Q is a compound of Formula I, or combinations thereof. In some embodiments, the inhibitor of POL Q is a compound disclosed herein, or combinations thereof. In some embodiments, the inhibitor of POL Q is a compound disclosed in Tables I, II, III, pharmaceutical salts thereof, or combinations thereof. In some embodiments, the inhibitor of POL Q is Compound 1 or a pharmaceutical salt thereof. In some embodiments, the inhibitor of POL Q is Compound 1

In some embodiments, the inhibitor of the MMEJ pathway in the composition comprising the eukaryotic cell is about 0.01 mM to about 1 mM, about 0.1 mM to about 1 mM, about 0.1 mM to about 0.5 mM, about 0.1 mM to about 100 mM, or about 1 mM to about 50 mM.

In some embodiments, the inhibitor of the NHEJ pathway is an inhibitor of DNA-dependent protein kinase (DNA-PK). In some embodiments, the inhibitor of DNA-PK is M3814, M9831/VX984, Nu7441, KU0060648, AZD7648, or combinations thereof. In some embodiments, the inhibitor of DNA-PK is AZD7648. In some embodiments, the inhibitor of DNA-PK is a peptide.

In some embodiments, the inhibitor of the NHEJ pathway in the composition comprising the eukaryotic cell is about 0.01 mM to about 1 mM, about 0.1 mM to about 1 mM, about 0.1 mM to about 0.5 mM, about 0.1 mM to about 100 mM, or about 1 mM to about 50 mM.

In some embodiments, the inhibitor of the MMEJ pathway is added to the composition comprising a eukaryotic cell comprising a genomically-integrated Cas polynucleotide 0 minutes to about 48 hours, 0 minutes to about 24 hours, 0 minutes to about 12 hours, 0 minutes to about 6 hours, or 0 minutes to about 1 hour before induction of the genomically-integrated Cas polynucleotide.

In some embodiments, the inhibitor of the NHEJ pathway is added to the composition comprising a eukaryotic cell comprising a genomically-integrated Cas polynucleotide 0 minutes to about 48 hours, 0 minutes to about 24 hours, 0 minutes to about 12 hours, 0 minutes to about 6 hours, or 0 minutes to about 1 hour before induction of the genomically-integrated Cas polynucleotide.

In some embodiments, the inhibitor of the MMEJ pathway and the inhibitor of the NHEJ pathway are added to the composition comprising the eukaryotic cell comprising a genomically-integrated Cas polynucleotide at the same time. In some embodiments, the inhibitor of the MMEJ pathway and the inhibitor of the NHEJ pathway are added to the composition comprising the eukaryotic cell comprising a genomically-integrated Cas polynucleotide at different times.

In some embodiments, the inhibitor of the MMEJ pathway is added to the composition comprising a eukaryotic cell comprising a genomically-integrated Cas polynucleotide at the same time as induction of the genomically-integrated Cas polynucleotide.

In some embodiments, the inhibitor of the NHEJ pathway is added to the composition comprising a eukaryotic cell comprising a genomically-integrated Cas polynucleotide at the same time as induction of the genomically-integrated Cas polynucleotide

In some embodiments, the inhibitor of the MMEJ pathway and the inhibitor of the NHEJ pathway are added to the composition comprising a eukaryotic cell comprising a genomically-integrated Cas polynucleotide at the same time as induction of the genomically-integrated Cas polynucleotide.

In some embodiments, the inhibitor of the MMEJ pathway is in the composition comprising the eukaryotic cell comprising a genomically-integrated Cas polynucleotide for about 1 to about 300 hours, about 10 to about 100 hours, or about 20 to about 80 hours.

In some embodiments, the inhibitor of the MMEJ pathway is added to the composition comprising the eukaryotic cell comprising a genomically-integrated Cas polynucleotide at least once, at least twice, or at least three times.

In some embodiments, the inhibitor of the NHEJ pathway is in the composition comprising the eukaryotic cell comprising a genomically-integrated Cas polynucleotide for about 1 to about 300 hours, about 10 to about 100 hours, or about 20 to about 80 hours.

In some embodiments, the inhibitor of the NHEJ pathway is added to the composition comprising the eukaryotic cell comprising a genomically-integrated Cas polynucleotide at least once, at least twice, or at least three times.

In some embodiments, the composition comprising the eukaryotic cell comprising a genomically-integrated Cas polynucleotide is a cell culture. In some embodiments, the cell cultures is an in vitro cell culture or an ex vivo cell culture.

In some embodiments, the eukaryotic cell comprising a genomically-integrated Cas polynucleotide is in vivo.

In some embodiments, the cell culture comprises a cell extract. In some embodiments, the cell culture is a mammalian cell culture.

In some embodiments, the eukaryotic cell comprising a genomically-integrated Cas polynucleotide is a lymphocyte. In some embodiments, the lymphocyte comprises a chimeric antigen receptor (CAR) or a T cell receptor (TCR).

In some embodiments, the eukaryotic cell comprising a genomically-integrated Cas polynucleotide is a pluripotent stem cell. In some embodiments, the pluripotent stem cell is an induced pluripotent stem cell (iPSC).

In some embodiments, the present disclosure relates to a method of inserting a polynucleotide of interest into a genome of a eukaryotic cell, the method comprising (a) adding an inhibitor of the microhomology-mediated end joining (MMEJ) pathway to a composition comprising the eukaryotic cell, and (b) adding to the composition comprising the eukaryotic cell (i) a Cas effector protein, (ii) a polynucleotide of interest, and (iii) a polynucleotide comprising an RNA guide sequence, a Cas-binding region, a DNA template sequence, or combinations thereof, wherein the polynucleotide of interest is inserted into the genome by homology directed repair (HDR) or single-stranded template repair (SSTR).

In some embodiments, the method comprises adding an inhibitor of the non-homologous end joining (NHEJ) pathway to the composition comprising the eukaryotic cell.

In some embodiments, the Cas effector protein and the polynucleotide comprising an RNA guide sequence, a Cas-biding region, a DNA template sequence, or combinations thereof, are added in the form of a ribonucleoprotein (RNP).

In some embodiments, the Cas effector protein is encoded by a Cas polynucleotide. In some embodiments, the Cas effector protein and the polynucleotide of interest are encoded on a vector. In some embodiments, the Cas effector protein and the polynucleotide of (iii) are encoded on a vector. In some embodiments, the Cas effector protein, the polynucleotide of interest, and the polynucleotide of (iii) are encoded on a vector. In some embodiments, the polynucleotide is on a vector.

In some embodiments, the present disclosure relates to a method of increasing the efficiency of homology directed repair (HDR) and single-stranded template repair (SSTR) gene insertions in a eukaryotic cell, the method comprising adding an inhibitor of the microhomology-mediated end joining (MMEJ) pathway when performing CRISPR/Cas-mediated gene insertions in the eukaryotic cell.

In some embodiments, the method further comprises adding an inhibitor of the non-homologous end joining (NHEJ) pathway.

In some embodiments, the CRISPR/Cas-mediated gene insertion is a CRISPR/Cas9-mediated gene insertion.

In some embodiments, the present disclosure relates to a method of reducing microhomology-mediated end joining (MMEJ) pathway recombination during CRISPR/Cas-mediated gene insertion in a cell, the method comprising adding an inhibitor of the MMEJ pathway to the cell when performing Cas-mediated gene insertions.

In some embodiments, the method further comprises reducing non-homologous end joining (NHEJ) recombination during CRISPR/Cas-mediated gene insertions in a cell comprising adding an inhibitor of the NHEJ pathway to the cell.

In some embodiments, the CRISPR/Cas-mediated gene insertions are CRISPR/Cas9-mediated gene insertions.

In some embodiments, the present disclosure relates to a composition comprising a Cas effector protein or a vector encoding a Cas effector protein, and an inhibitor of the microhomology-mediated end joining (MMEJ) pathway. In some embodiments, the composition further comprises an inhibitor of the non-homologous end joining (NHEJ) pathway.

In some embodiments, the composition further comprises a polynucleotide comprising at least one RNA guide sequence, a Cas-binding region, a DNA template sequence, or combinations thereof.

In some embodiments, the Cas effector protein is a Cas9 nuclease, a Cas12a nuclease, or a Cas12f nuclease. In some embodiments the Cas effector protein is a Cas9 nuclease. In some embodiments, the Cas9 nuclease is a Cas9 nuclease fused to a reverse transcriptase, a Cas9 nuclease fused to a DNA polymerase, a Cas9 fused to DNlS, a Cas9 nickase, a Cas9 fused to a Geminin degron domain, or a Cas9 nuclease fused to CTIP.

In some embodiments, the vector encoding the Cas effector protein is a viral vector.

In some embodiments, the polynucleotide comprising at least one RNA guide sequence, a Cas-binding region, a DNA template sequence, or combinations thereof, is encoded on a vector.

In some embodiments the vector is a viral vector.

In some embodiments, the Cas effector protein and the polynucleotide comprising at least one RNA guide sequence, a Cas-binding region, a DNA template sequence, or combinations thereof, are in the form of a ribonucleoprotein (RNP).

In some embodiments, the composition further comprises a pharmaceutically acceptable carrier, diluent, or excipient.

In some embodiments, the present disclosure relates to a kit comprising a Cas effector protein or a vector encoding a Cas effector protein and an inhibitor of the microhomology-mediated end joining (MMEJ) pathway.

In some embodiments, the kit further comprises an inhibitor of the non-homologous end-joining (NHEJ) pathway.

In some embodiments, the kit further comprises a polynucleotide comprising at least one RNA guide sequence, a Cas-binding region, a DNA template sequence, or combinations thereof.

In some embodiments, the Cas effector protein is a Cas9 nuclease, a Cas12a nuclease, or a Cas12f nuclease. In some embodiments, the Cas effector protein is a Cas9 nuclease. In some embodiments, the Cas9 nuclease is a Cas9 nuclease fused to a reverse transcriptase, a Cas9 fused to a DNA polymerase, a Cas9 fused to DNlS, a Cas9 nickase, a Cas9 fused to a Geminin degron domain, or a Cas9 nuclease fused to CTIP.

In some embodiments, the polynucleotide comprising at least one RNA guide sequence, a Cas-binding region, a DNA template sequence, or combinations thereof, is encoded on a vector. In some embodiments, the vector is a viral vector.

In some embodiments, the Cas effector protein and the polynucleotide comprising at least one RNA guide sequence, a Cas-binding region, a DNA template sequence, or combinations thereof, are in the form of a ribonucleoprotein (RNP).

### BRIEF DESCRIPTION OF THE DRAWINGS

FIG. 1 is a schematic showing manipulation of DNA repair with small molecule inhibitors. In this schematic, components of a CRISPR/Cas genome editing system provide double stranded breaks (DSB) at specific sequences. The DSB can be repaired by the imprecise and error-prone microhomology-mediated end joining (MMEJ) or non-homologous end joining (NHEJ) pathways, or alternatively, by the more precise homology directed repair (HDR) pathway.

FIG. 2A-2B illustrate an exemplary method described in embodiments herein. FIG. 2A shows an example in which cells are pre-treated for 3 hours with pharmacological inhibitors of POL Q/DNA polymerase q (PolQ) and/or DNA-dependent protein kinase (DNA-PK). A CRISPR/Cas gene editing system is then added to the cells. After 60 hours, genomic DNA is isolated from the cells and deep-targeted sequencing is performed. The results of the sequencing are then analyzed by Rational InDel Meta-Analysis (RIMA) in order to determine the frequency of MMEJ and NHEJ repairs. FIG. 2B shows a graphical representation of the RIMA results, where deletions associated with microhomologies are visualized according to the bars shown in the figure.

FIG. 3 shows the effect of inhibiting the MMEJ and NHEJ pathways on DNA repair of DSB and precise integrations as described in Example CRISPR-1.

FIG. 4 shows the effect of MMEJ and NHEJ pathway inhibition on CRISPR/Cas editing efficiency as described in Example CRISPR-1.

FIG. 5 shows the effect of MMEJ and NHEJ pathway inhibition on CRISPR/Cas-mediated gene knock-in efficiency in mutated sequencing reads as described in Example CRISPR-2.

FIG. 6 shows the effect of MMEJ and NHEJ pathway inhibition on CRISPR/Cas-mediated gene knock-in efficiency in mapped sequencing reads as described in Example CRISPR-2.

FIG. 7 shows the effect of Pol Q and DNA-PK inhibition on MMEJ in mutated reads as described in Example CRISPR-3.

FIG. 8 shows the effect of Pol Q inhibition on MMEJ in mapped reads as described in Example CRISPR-3.

FIG. 9 shows the effect of MMEJ and NHEJ pathway inhibition on cell confluency as described in Example CRISPR-4.

FIG. 10 shows the effect of MMEJ and NHEJ pathway inhibition on transfection efficiency as described in Example CRISPR-4.

FIGS. 11 & 12 show the effect of inhibiting the MMEJ and NHEJ pathways on DNA repair of DSB and precise integration in induced Pluripotent Stem Cells (iPSC).

FIG. 13 shows the effect of Pol Q and DNA-PK inhibition on DNA repair of CRISPR/Cas-induced DSB in Cas9-inducible iPSCs.

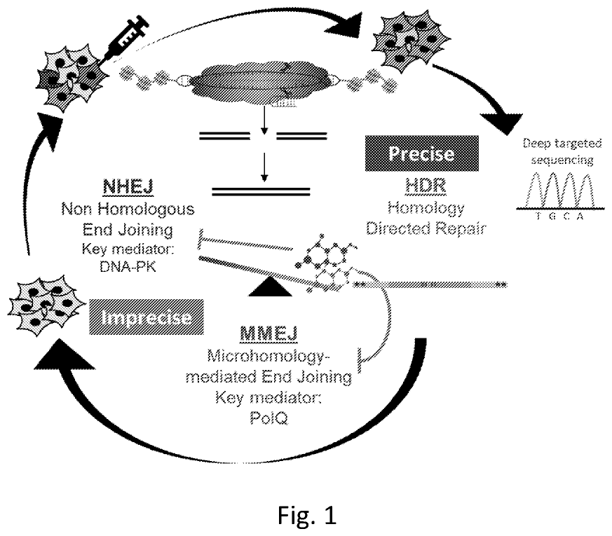

### DETAILED DESCRIPTION

The present disclosure relates to methods of improving CRISPR/Cas-mediated gene insertion (i.e. gene “knock-in”) in eukaryotic cells, compositions for improved CRISPR/Cas-mediated insertion, and kits for improved CRISPR/Cas-mediated gene insertion. In general a CRISPR system, e.g., a CRISPR/Cas system, includes elements that promote the formation of a CRISPR complex, such as a guide polynucleotide and a Cas protein, at the site of a target polynucleotide, e.g., a target DNA sequence. In naturally-occurring CRISPR systems (e.g., the bacterial immunity CRISPR/Cas9 system), foreign DNA is incorporated into CRISPR arrays, which then produce CRISPR-RNAs (crRNA). The crRNA includes RNA guide sequence regions complementary to the foreign DNA site and hybridizes with trans-activating CRISPR-RNA (tracrRNA), which is also encoded by the CRISPR system. The tracrRNA forms secondary structures, e.g., stem loops, and is capable of binding to Cas9 protein. The crRNA/tracrRNA hybrid associates with Cas9, and the crRNA/tracrRNA/Cas9 complex recognizes and cleaves foreign DNA bearing the protospacer sequences, thereby conferring immunity against the invading virus or plasmid. CRISPR/Cas systems are further described in, e.g., Jinek et al., Science 337(6096):816-821 (2012); Cong et al., Science 339(6121):819-823 (2013); Mali et al., Science 339(6121):823-826 (2013); and Sander et al., Nat Biotechnol 32:347-355 (2014).

CRISPR/Cas systems have been engineered to introduce insertions into a target polynucleotide, also known as targeted insertions. Typically, the guide polynucleotide is designed such that the Cas protein generates a double-stranded cleavage at the target polynucleotide, and a separate donor template comprising the sequence of interest is inserted into the cleaved target polynucleotide by cellular DNA repair mechanisms, e.g., non-homologous end joining (NHEJ) or homology directed repair (HDR). The efficiency of insertion is dependent on several factors, including transfection ratio of the donor template, Cas protein, and guide polynucleotide; sequence and size of the donor template; and type of DNA repair mechanism triggered. For example, HDR provides high-fidelity DNA repair but has low insertion frequency, while NHEJ has higher insertion frequency but may also introduce mutations into the target DNA.

In some embodiments, the present disclosure provides compositions, polynucleotides, and/or fusion proteins for improved targeted insertion methods. In some embodiments, the compositions, polynucleotides, and/or fusion proteins of the present disclosure provide higher precision of inserting a sequence of interest. In some embodiments, the compositions, polynucleotides, and fusion proteins of the present disclosure provide higher efficiency of inserting a sequence of interest.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps. It is contemplated that any embodiment discussed in this specification can be implemented with respect to any protein, compositions, polynucleotides, vectors, cells, methods, and/or kits of the present disclosure. Furthermore, compositions, polynucleotides, vectors, cells, and/or kits of the present disclosure can be used to achieve methods and proteins of the present disclosure.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

A “nucleic acid,” “nucleic acid molecule,” “nucleotide,” “nucleotide sequence,” “oligonucleotide,” or “polynucleotide” means a polymeric compound including covalently linked nucleotides. The term “nucleic acid” includes ribonucleic acid (RNA) or deoxyribonucleic acid (DNA) both of which may be single- or double-stranded. The polynucleotide may comprise naturally-occurring nucleobases (e.g., guanine, adenine, cytosine, thymine, and uracil), modified nucleobases (e.g., hypoxanthine, xanthine, 7-methylguanine, dihydrouracil, 5-methylcytosine, 5-hydroxymethylcytosine), and/or artificial nucleobases (e.g., isoguanine or isocytosine). Nucleic acids are transcribed from a 5′ end to a 3′ end. In some embodiments, the disclosure provides a polynucleotide comprising RNA and DNA nucleotides. Methods of producing a polynucleotide comprising both RNA and DNA nucleotides are known in the art and include, e.g., ligation or oligonucleotide synthesis methods. In some embodiments, the disclosure provides a polynucleotide capable of forming a complex with a Cas nuclease or Cas nickase as described herein. In some embodiments, the disclosure provides a polynucleotide encoding any one of the proteins disclosed herein, e.g., a Cas nuclease or Cas nickase.

A “gene” refers to an assembly of nucleotides that encode a polypeptide and includes cDNA and genomic DNA nucleic acid molecules. In some embodiments, “gene” also refers to a non-coding nucleic acid fragment that can act as a regulatory sequence preceding (i.e., 5′) and following (i.e., 3′) the coding sequence.

A nucleic acid molecule is “hybridizable” or “hybridized” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are known and exemplified in Sambrook et al., Molecular Cloning: A Laboratory Manual, Second Edition, Cold Spring Harbor Laboratory Press, Cold Spring Harbor (1989), particularly Chapter 11 and Table 11.1 therein. The conditions of temperature and ionic strength determine the stringency of the hybridization. The stringency of the hybridization conditions can be selected to provide selective formation or maintenance of a desired hybridization product of two complementary polynucleotides, in the presence of other potentially cross-reacting or interfering polynucleotides. Stringent conditions are sequence-dependent; typically, longer complementary sequences specifically hybridize at higher temperatures than shorter complementary sequences. Generally, stringent hybridization conditions are between about 5° C. to about 10° C. lower than the thermal melting point (Tₘ) (i.e., the temperature at which 50% of the sequences hybridize to a substantially complementary sequence) for a specific polynucleotide at a defined ionic strength, concentration of chemical denaturants, pH, and concentration of the hybridization partners. Generally, nucleotide sequences having a higher percentage of G and C bases hybridize under more stringent conditions than nucleotide sequences having a lower percentage of G and C bases. Generally, stringency can be increased by increasing temperature, increasing pH, decreasing ionic strength, and/or increasing the concentration of chemical nucleic acid denaturants (such as formamide, dimethylformamide, dimethylsulfoxide, ethylene glycol, propylene glycol and ethylene carbonate). Stringent hybridization conditions typically include salt concentrations or ionic strength of less than about 1 M, 500 mM, 200 mM, 100 mM or 50 mM; hybridization temperatures above about 20° C., 30° C., 40° C., 60° C. or 80° C.; and chemical denaturant concentrations above about 10%, 20%, 30% 40% or 50%. Because many factors can affect the stringency of hybridization, the combination of parameters may be more significant than the absolute value of any parameter alone.

The term “complementary” is used to describe the relationship between nucleotide bases that are capable of hybridizing to one another. For example, with respect to DNA, adenosine is complementary to thymine and cytosine is complementary to guanine. When two nucleic acids are “complementary,” it is meant that a first nucleic acid or one or more regions thereof is capable of hydrogen bonding with a second nucleic acid or one or more regions thereof. Complementary nucleic acids need not have complementarity at each nucleotide and may include one or more nucleotide mismatches, i.e., points at which hydrogen bonding does not occur. For example, complementary oligonucleotides can have at least 80%, at least 85%, at least 90%, at least 95%, at least 96%, at least 97%, at least 98%, or at least 99% of nucleotides hydrogen bond. By contrast, “fully complementary” or “100% complementary” in reference to oligonucleotides means that each nucleotide hydrogen bonds without any nucleotide mismatches.

The term “homologous recombination” refers to the insertion of an exogenous polynucleotide (e.g., DNA) into another nucleic acid (e.g., DNA) molecule, e.g., insertion of a vector, polynucleotide fragment or gene in a chromosome. In some cases, the exogenous polynucleotide targets a specific chromosomal site for homologous recombination. For specific homologous recombination, the exogenous polynucleotide typically contains sufficiently long regions of homology to sequences of the chromosome to allow complementary binding and incorporation of the exogenous polynucleotide into the chromosome. Longer regions of homology and greater degrees of sequence similarity may increase the efficiency of homologous recombination. In some embodiments, the polynucleotides or compositions described herein facilitate homologous recombination by generating breaks, e.g., double-stranded breaks in a nucleic acid sequence.

The term “homology-directed repair” or “HDR” refers to a mechanism of repairing double-stranded breaks in DNA using a template nucleic acid sequence. The most common form of HDR is homologous recombination. In HDR, a double-stranded break is repaired by a process involving resection of the 5′ ended DNA strand at the break to create a 3′ overhang, which serves as both a substrate for proteins required for strand invasion and as a primer for DNA repair synthesis. The invasive strand then displaces one strand of a double-stranded DNA template sequence which comprises homologous sequences and pair with the other strand, resulting in the formation of hybrid DNA known as the displacement loop. These recombination intermediates are then resolved to complete the DNA repair process.

The term “single-strand template repair” or “SSTR” refers to another mechanism of repairing double-stranded breaks in DNA using a template nucleic acid sequence. In contrast to HDR, SSTR utilizes a single-stranded template nucleic acid sequence for double-strand DNA break repair.

The term “non-homologous end joining pathway” or “NHEJ pathway” refers to another mechanism of repairing double-stranded breaks in DNA. In NHEJ, a Ku80/70 heterodimer recognizes and binds to blunt ends formed by the double-stranded break, where the resulting complex activates the activity of DNA-PK. Activation of DNA-PK recruits Artemis nuclease, DNA polymerases, and DNA ligases to ultimately repair the double-stranded break. NHEJ differs from HDR and homologous recombination that that it does not require a homologous template sequence for repair.

The term “microhomology-mediated end joining pathway” or “NMEJ pathway” refers to another mechanism for repairing double-stranded breaks in DNA. MMEJ is similar to NHEJ in that a homologous template sequence is not utilized for double-stranded break repair. However, MMEJ is distinguished from other repair mechanisms by its utilization of microhomologous sequences to align broken DNA strands. MMEJ does not rely on Ku protein or DNA-PK, but DNA polymerase q (Pol Q) has been shown to be required for MMEJ. MMEJ is also known as “alternative end-joining,” or “alternative nonhomologous end-joining” or “Alt-NHEJ.”

As used herein, the term “operably linked” means that a polynucleotide of interest, e.g., the polynucleotide encoding a nuclease, is linked to the regulatory element in a manner that allows for expression of the polynucleotide. Regulatory elements can be cis-regulatory elements or trans-regulatory elements. Regulatory elements include, for example, promoters, enhancers, terminators, 5′ and 3′ UTRs, insulators, silencers, operators, and the like. In some embodiments, the regulatory element is a promoter. In some embodiments, a polynucleotide expressing a protein of interest is operably linked to a promoter on an expression vector.

As used herein, “promoter,” “promoter sequence,” or “promoter region” refers to a DNA regulatory region or polynucleotide capable of binding RNA polymerase and involved in initiating transcription of a downstream coding or non-coding sequence. In some embodiments, the promoter sequence includes the transcription initiation site and extends upstream to include the minimum number of bases or elements used to initiate transcription at levels detectable above background. In some embodiments, the promoter sequence includes a transcription initiation site, as well as protein binding domains responsible for the binding of RNA polymerase. Eukaryotic promoters typically contain “TATA” boxes and “CAT” boxes. Various promoters, including inducible promoters, may be used to drive expression of the various vectors of the present disclosure.

A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. In some embodiments, the vector is an episomal vector, which is removed/lost from a population of cells after a number of cellular generations, e.g., by asymmetric partitioning. The term “vector” includes both viral and non-viral means for introducing the nucleic acid into a cell in vitro, ex vivo, or in vivo. A large number of vectors known in the art may be used to manipulate nucleic acids, incorporate response elements and promoters into genes, etc. A vector may include one or more regulatory regions, and/or selectable markers useful in selecting, measuring, and monitoring nucleic acid transfer results (transfer to which tissues, duration of expression, etc.).

Possible vectors include, for example, plasmids or modified viruses including, for example, bacteriophages such as lambda derivatives, or plasmids such as PBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified, or any site may be produced by ligating polynucleotides (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include, but are not limited, to retrovirus, lentivirus, adenovirus, adeno-associated virus, pox, baculovirus, vaccinia, herpes simplex, Epstein-Barr, adenovirus, geminivirus, and caulimovirus vectors. In some embodiments, a viral vector is utilized to provide the polynucleotides described herein. In some embodiments, a viral vector is utilized to provide a polynucleotide coding for a protein described herein.

Vectors may be introduced into the desired host cells by known methods, including, but not limited to, transfection, transduction, cell fusion, and lipofection. Vectors can include various regulatory elements including promoters. In some embodiments, vector designs can be based on constructs designed by Mali et al., Nat Methods 10: 957-63 (2013).

Methods known in the art may be used to propagate polynucleotides and/or vectors provided herein. Once a suitable host system and growth conditions are established, recombinant expression vectors can be propagated and prepared in quantity. As described herein, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors.

The term “plasmid” refers to an extra chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of polynucleotides have been joined or recombined into a unique construction which is capable of introducing a promoter fragment and DNA sequence for a selected gene product along with appropriate 3′ untranslated sequence into a cell. In some embodiments, a plasmid is utilized to provide the polynucleotides described herein. In some embodiments, a plasmid is utilized to provide a polynucleotide coding for a protein described herein.

The term “transfection” as used herein means the introduction of an exogenous nucleic acid molecule, including a vector, into a cell. Transfection methods, e.g., for components of the CRISPR/Cas compositions described herein, are known to one of ordinary skill in the art. A “transfected” cell includes an exogenous nucleic acid molecule inside the cell and a “transformed” cell is one in which the exogenous nucleic acid molecule within the cell induces a phenotypic change in the cell. The transfected nucleic acid molecule can be integrated into the host cell's genomic DNA and/or can be maintained by the cell, temporarily or for a prolonged period of time, extra-chromosomally. Host cells or organisms that express exogenous nucleic acid molecules or fragments are referred to herein as “recombinant,” “transformed,” or “transgenic” organisms. In some embodiments, the present disclosure provides a host cell comprising any of the vectors described herein, e.g., a vector comprising a Cas polynucleotide, a vector comprising the polynucleotide of interest, or a vector comprising a polynucleotide comprising an RNA guide sequence, a CAS-binding region, a DNA Template sequence or combinations thereof.

The term “host cell” refers to a cell into which a recombinant expression vector has been introduced, or “host cell” may also refer to the progeny of such a cell. Because modifications may occur in succeeding generations, for example, due to mutation or environmental influences, the progeny may not be identical to the parent cell, but are still included within the scope of the term “host cell.”

The terms “peptide,” “polypeptide,” and “protein” are used interchangeably herein, and refer to a polymeric form of amino acids of any length, which can include coded and non-coded amino acids, non-naturally occurring amino acids, chemically or biochemically modified or derivatized amino acids, peptides and polypeptides having modified peptide backbones, and circular/cyclic peptides and polypeptides.

The start of the protein or polypeptide is known as the “N-terminus” (and also referred to as the amino-terminus, NH₂-terminus, N-terminal end or amine-terminus), referring to the free amine (—NH₂) group of the first amino acid residue of the protein or polypeptide. The end of the protein or polypeptide is known as the “C-terminus” (and also referred to as the carboxy-terminus, carboxyl-terminus, C-terminal end, or COOH-terminus), referring to the free carboxyl group (—COOH) of the last amino acid residue of the protein or polypeptide.

An “amino acid” as used herein refers to a compound including both a carboxyl (—COOH) and amino (—NH₂) group. “Amino acid” refers to both natural and unnatural, i.e., synthetic, amino acids. Natural amino acids, with their three-letter and single-letter abbreviations, include: alanine (Ala; A); arginine (Arg, R); asparagine (Asn; N); aspartic acid (Asp; D); cysteine (Cys; C); glutamine (Gln; Q); glutamic acid (Glu; E); glycine (Gly; G); histidine (His; H); isoleucine (Ile; I); leucine (Leu; L); lysine (Lys; K); methionine (Met; M); phenylalanine (Phe; F); proline (Pro; P); serine (Ser; S); threonine (Thr; T); tryptophan (Trp; W); tyrosine (Tyr; Y); and valine (Val; V). Unnatural or synthetic amino acids include a side chain that is distinct from the natural amino acids provided above and may include, e.g., fluorophores, post-translational modifications, metal ion chelators, photocaged and photocross-linking moieties, uniquely reactive functional groups, and NMR, IR, and x-ray crystallographic probes. Exemplary unnatural or synthetic amino acids are provided in, e.g., Mitra et al., Mater Methods 3:204 (2013) and Wals et al., Front Chem 2:15 (2014). Unnatural amino acids may also include naturally-occurring compounds that are not typically incorporated into a protein or polypeptide, such as, e.g., citrulline (Cit), selenocysteine (Sec), and pyrrolysine (Pyl).

An “amino acid substitution” refers to a polypeptide or protein including one or more substitutions of wild-type or naturally occurring amino acid with a different amino acid relative to the wild-type or naturally occurring amino acid at that amino acid residue. The substituted amino acid may be a synthetic or naturally occurring amino acid. In some embodiments, the substituted amino acid is a naturally occurring amino acid selected from the group consisting of: A, R, N, D, C, Q, E, G, H, I, L, K, M, F, P, S, T, W, Y, and V. In some embodiments, the substituted amino acid is an unnaturally or synthetic amino acid. Substitution mutants may be described using an abbreviated system. For example, a substitution mutation in which the fifth (5^{th}) amino acid residue is substituted may be abbreviated as “X5Y,” wherein “X” is the wild-type or naturally occurring amino acid to be replaced, “5” is the amino acid residue position within the amino acid sequence of the protein or polypeptide, and “Y” is the substituted, or non-wild-type or non-naturally occurring, amino acid.

An “isolated” polypeptide, protein, peptide, or nucleic acid is a molecule that has been removed from its natural environment. It is also understood that “isolated” polypeptides, proteins, peptides, or nucleic acids may be formulated with excipients such as diluents or adjuvants and still be considered isolated. As used herein, “isolated” does not necessarily imply any particular level purity of the polypeptide, protein, peptide, or nucleic acid.

The term “recombinant” when used in reference to a nucleic acid molecule, peptide, polypeptide, or protein means of, or resulting from, a new combination of genetic material that is not known to exist in nature. A recombinant molecule can be produced by any of the techniques available in the field of recombinant technology, including, but not limited to, polymerase chain reaction (PCR), gene splicing (e.g., using restriction endonucleases), and solid-phase synthesis of nucleic acid molecules, peptides, or proteins.

The term “exogenous” means that the referenced molecule or activity introduced into the host cell. The molecule can be introduced, for example, by introduction of an encoding nucleic acid into the host genetic material, such as by integration into a host chromosome or as non-chromosomal genetic material, e.g., a plasmid. An “exogenous” protein can be introduced into a host cell via an “exogenous” nucleic acid encoding the protein. The term “endogenous” refers to a referenced molecule or activity that is naturally present in the host cell. An “endogenous” protein is expressed by a nucleic acid contained within the host cell. The term “heterologous” refers to a molecule or activity derived from a source other than the referenced organism/species, whereas “homologous” refers to a molecule or activity derived from the host organism/species. Accordingly, exogenous expression of an encoding nucleic acid can utilize either or both of a heterologous or homologous encoding nucleic acid.

The term “domain” when used in reference to a polypeptide or protein means a distinct functional and/or structural unit in a protein. Domains are sometimes responsible for a particular function or interaction, contributing to the overall role of a protein. Domains may exist in a variety of biological contexts. Similar domains may be found in proteins with different functions. Alternatively, domains with low sequence identity (i.e., less than about 50%, less than about 40%, less than about 30%, less than about 20%, less than about 10%, less than about 5%, or less than about 1% sequence identity) may have the same function.

The term “motif,” when used in reference to a polypeptide or protein, generally refers to a set of conserved amino acid residues, typically shorter than 20 amino acids in length, that may be important for protein function. Specific sequence motifs may mediate a common function, such as protein-binding or targeting to a particular subcellular location, in a variety of proteins. Examples of motifs include, but are not limited to, nuclear localization signals, microbody targeting motifs, motifs that prevent or facilitate secretion, and motifs that facilitate protein recognition and binding. Motif databases and/or motif searching tools are known in the field and include, for example, PROSITE, PFAM, PRINTS, and MiniMotif Miner.

An “engineered” protein, as used herein, means a protein that includes one or more modifications in a protein to achieve a desired property. Exemplary modifications include, but are not limited to, insertion, deletion, substitution, and/or fusion with another domain or protein. A “fusion protein” (also termed “chimeric protein”) is a protein comprising at least two domains, typically coded by two separate genes, that have been joined such that they are transcribed and translated as a single unit, thereby producing a single polypeptide having the functional properties of each of the domains. Engineered proteins of the present disclosure include Cas nucleases, Cas nickases, and fusions of Cas proteins with a DNA polymerase, DNA ligase, and/or DNA polymerase-binding protein.

In some embodiments, engineered protein is generated from a wild-type protein. As used herein, a “wild-type” protein or nucleic acid is a naturally-occurring, unmodified protein or nucleic acid. For example, a wild-type Cas9 protein can be isolated from the organism Streptococcus pyogenes. Wild-type can be contrasted with “mutant,” which includes one or more modifications in the amino acid and/or nucleotide sequence of the protein or nucleic acid. In some embodiments, an engineered protein can have substantially the same activity as a wild-type protein, e.g., greater than about 80%, greater than about 85%, greater than about 90%, greater than about 95%, or greater than about 99% of the activity as a wild-type protein. In some embodiments, the Cas nuclease of a fusion protein described herein has substantially the same activity as a wild-type Cas nuclease.

In some embodiments, an engineered protein, e.g., a Cas9 protein, can have substantially the same amino acid sequence as a wild-type protein, e.g., greater than about 80%, greater than about 85%, greater than about 90%, greater than about 95%, or greater than about 99% identify as a wild-type protein. As used herein, the terms “sequence similarity” or “% similarity” refers to the degree of identity or correspondence between nucleic acid sequences or amino acid sequences. In the context of polynucleotides, “sequence similarity” may refer to nucleic acid sequences where changes in one or more nucleotide bases results in substitution of one or more amino acids, but do not affect the functional properties of the protein encoded by the polynucleotide. “Sequence similarity” may also refer to modifications of the polynucleotide, such as deletion or insertion of one or more nucleotide bases, that do not substantially affect the functional properties of the resulting transcript. It is therefore understood that the present disclosure encompasses more than the specific exemplary sequences. Methods of making nucleotide base substitutions are known, as are methods of determining the retention of biological activity of the encoded polypeptide.

Moreover, the skilled artisan recognizes that similar polynucleotides encompassed by the present disclosure are also defined by their ability to hybridize, under stringent conditions, with the sequences exemplified herein. Similar polynucleotides of the present disclosure are about 70%, at least about 70%, about 75%, at least about 75%, about 80%, at least about 80%, about 85%, at least about 85%, about 90%, at least about 90%, about 95%, at least about 95%, about 99%, at least about 99%, or about 100% identical to the polynucleotides disclosed herein.

In the context of polypeptides, “sequence similarity” refers to two or more polypeptides where greater than about 40% of the amino acids are identical, or greater than about 60% of the amino acids are functionally identical. “Functionally identical” or “functionally similar” amino acids have chemically similar side chains. For example, amino acids can be grouped in the following manner according to functional similarity: (i) positively-charged side chains: Arg, His, Lys; (ii) negatively-charged side chains: Asp, Glu; (iii) polar, uncharged side chains: Ser, Thr, Asn, Gln; (iv) hydrophobic side chains: Ala, Val, Ile, Leu, Met, Phe, Tyr, Trp; and (v) others: Cys, Gly, Pro.

In some embodiments, similar polypeptides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical amino acids. In some embodiments, similar polypeptides of the present disclosure have about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% functionally identical amino acids.

Sequence similarity can be determined by sequence alignment using methods known in the field, such as, for example, BLAST, MUSCLE, Clustal (including ClustalW and ClustalX), and T-Coffee (including variants such as, for example, M-Coffee, R-Coffee, and Expresso).

Percent identity of polynucleotides or polypeptides can be determined when the polynucleotide or polypeptide sequences are aligned over a specified comparison window. In some embodiments, only specific portions of two or more sequences are aligned to determine sequence identity. In some embodiments, only specific domains of two or more sequences are aligned to determine sequence similarity. A comparison window can be a segment of at least 10 to over 1000 residues, at least 20 to about 1000 residues, or at least 50 to 500 residues in which the sequences can be aligned and compared. Methods of alignment for determination of sequence identity are well-known and can be performed using publicly available databases such as BLAST. For example, in some embodiments, “percent identity” of two amino acid sequences is determined using the algorithm of Karlin and Altschul, Proc Nat Acad Sci USA 87:2264-2268 (1990), modified as in Karlin and Altschul, Proc Nat Acad Sci USA 90:5873-5877 (1993). Such algorithms are incorporated into BLAST programs, e.g., BLAST+ or the NBLAST and XBLAST programs described in Altschul et al., J Mol Biol, 215: 403-410 (1990). BLAST protein searches can be performed with programs such as, e.g., the XBLAST program, score=50, wordlength=3 to obtain amino acid sequences homologous to the protein molecules of the disclosure. Where gaps exist between two sequences, Gapped BLAST can be utilized as described in Altschul et al., Nucleic Acids Res 25(17): 3389-3402 (1997). When utilizing BLAST and Gapped BLAST programs, the default parameters of the respective programs (e.g., XBLAST and NBLAST) can be used.

In some embodiments, a polypeptide or polynucleotide has 70%, at least 70%, 75%, at least 75%, 80%, at least 80%, 85%, at least 85%, 90%, at least 90%, 95%, at least 95%, 97%, at least 97%, 98%, at least 98%, 99%, or at least 99% or 100% sequence identity with a reference polypeptide or polynucleotide (or a fragment of the reference polypeptide or polynucleotide) provided herein. In some embodiments, a polypeptide or polynucleotide have about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99% or about 100% sequence identity with a reference polypeptide or polynucleotide (or a fragment of the reference polypeptide or nucleic acid molecule) provided herein.

As used herein, a “complex” refers to a group of two or more associated polynucleotides and/or polypeptides. In the context of complex formation, the terms “associate” or “association” refers to molecules bound to one another through electrostatic, hydrophobic/hydrophilic, and/or hydrogen bonding interaction, without being covalently attached. A molecule that comprises different moieties covalently attached to one another is known. In some embodiments, a complex is formed when all the components of the complex are present together, i.e., a self-assembling complex. In some embodiments, a complex is formed through chemical interactions between different components of the complex such as, for example, hydrogen-bonding. In some embodiments, the polynucleotides provided herein form a complex with the proteins provided herein through secondary structure recognition of the polynucleotide by the protein. In some embodiments, the Cas-binding region of the polynucleotides provided herein comprise a secondary structure recognized by a Cas nuclease, Cas nickase, or fusion protein provided herein.

The term “alkoxy” refers to an alkyl group attached to the rest of the molecule via an oxygen atom. Representative alkoxy groups include, but are not limited to, methoxy, ethoxy, propoxy, tert-butoxy and the like.

The term “alkoxyalkyl” refers to an alkyl group attached to an alkoxy group, where in the group is attached to the rest of the molecule via a carbon on the alkyl group, i.e. a group having a structure of —R—O—R′ wherein R and R′ are the same or different alkyl groups.

The term “alkyl” or “alkane” refers to straight chained or branched non-aromatic hydrocarbon which is completely saturated. Examples of straight chained and branched alkyl groups include methyl, ethyl, n-propyl, iso-propyl, n-butyl, sec-butyl, tert-butyl, pentyl, hexyl, pentyl and octyl.

The term “alkylamino” refers to an amino group substituted with at least one alkyl group, i.e. a group having a structure of —NRR′, NHR, NRR¹H⁺, or NH₂R⁺ wherein R and R′ are the same or different alkyl groups.

The term “alkyne” or “alkynyl” is a non-aromatic hydrocarbon comprising at least one carbon-carbon triple bond. Examples of alkyne groups include acetylene, propyne, and butyne.

The term “amide” refers to a group with the general formula of RC(═O)NR1R2, or

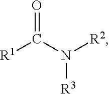

where R¹, R², and R³ are either hydrogen or the same or different alkyl groups, provided at least one is an alkyl group.

The term “carbamate” refers to a group with the general formula of R1OC(O)NR2R3 or

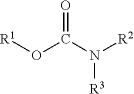

wherein R¹, R², and R³ are either hydrogen or the same or different alkyl groups, provided at least one is an alkyl group. The carbamate is connected to the rest of the molecule via a carbon on any of the alkyl groups.

The term “carbocycle” refers to a partially or completely saturated non-aromatic hydrocarbon ring system, including cycloalkyls, cycloalkenyls, and cycloalkynyls. Cycloalkyls include cyclopropane, cyclobutane, cyclopentane, cyclohexane, cyclopropene, cyclobutene, cyclopentene, and cyclohexene.

The term “ester” refers to a group having the structure R¹—C(O)—OR², or

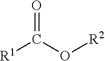

wherein R¹ and R² are the same or different alkyl groups. The ester is connected to the rest of the molecule via a carbon on either alkyl group.

The term “halo” means fluoro, chloro, bromo, and iodo. In some embodiments, halo is fluoro or chloro. In other embodiments, halo is fluoro. In still other embodiments, halo is chloro.

The term “haloalkyl” means an alkyl group in which one or more hydrogens has been substituted with a halo.

The term “hydroxyalkyl” means an alkyl group in which one or more hydrogens has been substituted with a hydroxy group.

The term “heterocycle” “heterocyclic” or “heterocyclyl” refers to a partially or completely saturated hydrocarbon ring system wherein at least one of the ring carbon atoms is replaced with a heteroatom independently selected from nitrogen, oxygen or sulphur. Heterocyclic groups can be attached to the rest of the molecule via a carbon or nitrogen ring-member atoms. Heterocycles include monocyclic heterocycles as well as spiro, fused and/or bridged polycyclic heterocycles such as bicyclic heterocycles. Examples of monocyclic heterocycles include, but are not limited to, tetrahydropyran, tetrahydrofuran, morpholine, azetidine, pyrrolidine, piperidine, piperazine, azepane, diazepane, oxetane, and isoxazolidine. Examples of polycyclic heterocycles include 2-azaspiro[3.3]heptane, 2,6-diazaspiro[3.3]heptane, 1,6-diazaspiro[3.3]heptane, 2-thia-6-azaspiro[3.3]heptane, 3,6-diazabicyclo[3.1.1]heptane, 2,6-diazaspiro[3.4]octane, 3,8-diazabicyclo[3.2.1]octane, and 4,7-diazaspiro[2.5]octane.

The term “sulfonyl” refers to a group having the general formula R¹S(O)₂R², or

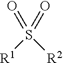

wherein R¹ and R² are either hydrogen or the same or different alkyl groups, provided at least one is an alkyl group. The sulfonyl is connected to the rest of the molecule via a carbon on either alkyl group.

In this specification the prefix C_{x-y} as used in terms such as “C_{x-y} alkyl” and the like where x and y are integers, indicates the numerical range of carbon atoms that are present in the group. Examples of suitable C₁₋₃ alkyl groups include, but are not limited to, methyl, ethyl, n-propyl, and i-propyl. Examples of suitable C₁₋₄ alkyl groups include, but are not limited to, methyl, ethyl, n-propyl, and i-propyl, n-butyl, i-butyl, s-butyl and t-butyl. In some cases, a group will have two sections comprising carbon, which case the prefix indicates the numerical range of total carbons in the group, e.g., C₂₋₆ alkoxyalkyl, refers to an alkoxyalkyl group wherein the alkyl group and the alkoxy group together have 2 to 6 carbons.

### Cas Proteins

As used herein, a “Cas effector protein,” also referred herein as “Cas protein” encompasses both Cas nucleases and Cas nickases. Cas effector proteins are part of the CRISPR/Cas system described herein. CRISPR/Cas systems, which include a Cas effector protein and a polynucleotide (also referred to as a “guide polynucleotide”), can be utilized for site-specific genome modifications. In some embodiments, the CRISPR/Cas system comprises a Cas effector protein and a guide polynucleotide comprising a Cas-binding region (which binds and/or activates the Cas protein) and a guide sequence (which hybridizes to a target sequence), where the Cas effector protein and the guide polynucleotide form a complex as described herein. In some embodiments, the CRISPR/Cas system comprises a Cas effector protein, a first polynucleotide comprising a guide sequence, and a second polynucleotide comprising a Cas-binding region, where the first and second polynucleotides hybridize to each other and form a complex with the Cas effector protein.

CRISPR/Cas systems can be classified as Types I to VI based on the Cas effector protein in the system. For example, Cas9 is found in Type II systems, and Cas12 is found in Type V systems. Each Type can be further divided into subtypes. For example, Type IT can include subtypes II-A, II-B, and II-C, and Type V can include subtypes V-A and V-B. Classification of CRISPR/Cas systems and Cas nucleases is further discussed in, e.g., Makarova et al., Methods Mol Biol 1311:47-75 (2015); Makarova et al., The CRISPR Journal Oct 2018; 325-336; and Koonin et al., Phil Trans R Soc B 374:20180087 (2018). Cas nucleases described herein can encompass any Type or variant, unless otherwise specified.

In some embodiments, the Cas effector protein is a Cas nuclease. In general, a Cas effector nuclease is capable of generating a double-stranded polynucleotide cleavage, e.g., a double-stranded DNA cleavage. In general, a Cas nuclease can include one or more nuclease domains, such as RuvC and HNH, and can cleave double-stranded DNA. In some embodiments, a Cas nuclease comprises a RuvC domain and an HNH domain, each of which cleaves one strand of double-stranded DNA. In some embodiments, the Cas nuclease generates blunt ends. In some embodiments, the RuvC and HNH of a Cas nuclease cleaves each DNA strand at the same position, thereby generating blunt ends. In some embodiments, the Cas nuclease generates cohesive ends. In some embodiments, the RuvC and HNH of a Cas nuclease cleaves each DNA strand at different positions (i.e., cut at an “offset”), thereby generating cohesive ends. As used herein, the terms “cohesive ends,” “staggered ends,” or “sticky ends” refer to a nucleic acid fragment with strands of unequal length. In contrast to “blunt ends,” cohesive ends are produced by a staggered cut on a double-stranded nucleic acid (e.g., DNA). A sticky or cohesive end has protruding singles strands with unpaired nucleotides, or “overhangs,” e.g., a 3′ or a 5′ overhang.

In some embodiments, the Cas nuclease is a Cas9 nuclease. Exemplary Cas9 nucleases include, but are not limited to, the Cas9 from Streptococcus pyogenes, Streptococcus thermophilus, Streptococcus mutans, Listeria innocua, Neisseria meningitidis, Staphylococcus aureus, Klebisella pneumoniae, and numerous other bacteria. Further exemplary Cas9 nucleases are described in, e.g., U.S. Pat. Nos. 8,771,945; 9,023,649; 10,000,772; 10,407,697; and US 2014/0068797. In some embodiments, the Cas9 nuclease is from S. pyogenes (SpCas9).

In some embodiments, the Cas9 nuclease comprises the sequence disclosed in UniProt ID G3ECR1 (SEQ ID NO: 1), UniProt ID Q99ZW2 (SEQ ID NO: 2), or UniProt ID J7RUA5 (SEQ ID NO: 3). In some embodiments, the Cas9 comprises a polypeptide sequence having at least 70%, at least 75%, at least 80%, at least 85%, at least 90%, at least 95%, at least 96%, at least 97%, at least 98%, at least 99%, or about 100% sequence identity to any of SEQ ID NOs: 1-3. In some embodiments, the disclosure provides for a polynucleotide which encodes a polypeptide having at least 70%, at least 75%, at least 80%, at least 85%, at least 90%, at least 95%, at least 96%, at least 97%, at least 98%, at least 99%, or about 100% sequence identity to any of SEQ ID NOs: 1-3. In some embodiments, the Cas9 is encoded by a polynucleotide which has been codon optimized for expression in a host cell.

In some embodiments, the Cas9 nuclease is a Type IIB Cas9 nuclease. In general, Type IIB Cas9 proteins are capable of generating cohesive ends, as described herein. Exemplary Type IIB Cas9 proteins include, but are not limited to, the Cas9 protein from Legionella pneumophila, Francisella novicida, Parasutterella excrementihominis, Sutterella wadsworthensis, Wolinella succinogenes, and numerous other bacteria. Further Type IIB Cas9 proteins are described in, e.g., WO 2019/099943.

In some embodiments, the Cas effector protein is a Cas12 nuclease. In some embodiments, the Cas nuclease is a Casl2a nuclease (formerly known as “Cpf1” or “C₂cl”). In some embodiments, the Cas nuclease is a Cas12f nuclease. Cas12f nuclease is also known in the art as Cas14 (Makarova et al, Nature Rev. Microbiol., 2019, 18:67-83). In some embodiments, the Cas nuclease is a Cas14 nuclease. Cas12 nucleases are generally smaller than Cas9 nucleases and can typically generate cohesive ends. Exemplary Cas12 proteins include, but are not limited to, the Cas12 protein from Francisella novicida, Acidaminococcus sp., Lachnospiraceae sp., Prevotella sp., and numerous other bacteria. Further Cas12 nuclease are described in, e.g., U.S. Pat. No. 9,580,701; US 2016/0208243; Zetsche et al., Cell 163(3):759-771 (2015); and Chen et al., Science 360:436-439 (2018).

In some embodiments, the Cas12 nuclease comprises the sequence disclosed by UniProt ID AOQ7Q2 SEQ ID NO: 4), UniProt ID U2UMQ6 (SEQ ID NO: 5), or UniProt ID TOD7A2 (SEQ ID NO: 6). In some embodiments, the Cas12 has at least 70%, at least 75%, at least 80%, at least 85%, at least 90%, at least 95%, at least 96%, at least 97%, at least 98%, at least 99%, or about 100% sequence identity to any of SEQ ID NOs: 4-6. In some embodiments, the disclosure provides for a polynucleotide which encodes a polypeptide having at least 70%, at least 75%, at least 80%, at least 85%, at least 90%, at least 95%, at least 96%, at least 97%, at least 98%, at least 99%, or about 100% sequence identity to the polypeptide of any of SEQ ID NOs: 4-6. In some embodiments, the Cas12 is encoded by a polynucleotide which has been codon optimized for expression in a host cell.

In some embodiments, the Cas effector protein is a Cas nickase. A nickase, which generates a single-stranded cleavage on a double-stranded polynucleotide (e.g., DNA), is distinguished from a nuclease, which cleaves both strands of a double-stranded polynucleotide (e.g., DNA). As discussed herein, a wild-type Cas nuclease typically comprises two catalytic nuclease domains, RuvC and HNH, and each nuclease domain is responsible for cleavage of one strand of double-stranded DNA. Thus, in some embodiments, a Cas nickase comprises an amino acid mutation in a catalytic domain relative to a Cas nuclease. Cas nickases are further described in, e.g., Cho et al., Genome Res 24:132-141 (2013); Ran et al., Cell 154:1380-1389 (2013); and Mali et al., Nat Biotechnol 31:833-838 (2013).

In some embodiments, the Cas nickase is a Cas9 nickase. In some embodiments, the Cas nickase is a Cas12a nickase. In some embodiments, the Cas nickase is a Type II-B Cas nickase. In some embodiments, the Cas nickase is produced by providing a mutation in a Cas nuclease. For example, the SpCas9 nickase comprises a D10A mutation or H840A mutation relative to wild-type SpCas9 nuclease. It will be understood by one of ordinary skill in the art that alignment methods such as those described herein can be used to determine the corresponding amino acid residues in other Cas nucleases (e.g., Cas12a or Type II-B Cas nucleases) to produce a Cas nickase.

In some embodiments, the Cas nuclease or Cas nickase of the composition is not fused to a heterologous protein domain. In some embodiments, the Cas nuclease or Cas nickase is not fused to a DNA polymerase, a DNA ligase, or a reverse transcriptase.

In some embodiments, the recombinant Cas effector proteins of the present disclosure are part of a fusion protein including one or more heterologous protein domains (e.g., about or at least about 1, 2, 3, 4, 5, 6, 7, 8, 9, or 10 or more domains in addition to the recombinant Cas effector protein). A Cas fusion protein can include any additional protein sequence, and optionally a linker sequence between any two domains. Examples of protein domains that may be fused to a recombinant Cas9 protein include, without limitation: epitope tags, reporter gene sequences, and protein domains having one or more of the following activities: methylase activity, demethylase activity, transcription activation activity, transcription repression activity, transcription release factor activity, histone modification activity, RNA cleavage activity, and nucleic acid binding activity. Non-limiting examples of epitope tags include: histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), autofluorescent proteins including blue fluorescent protein (BFP), and mCherry. In some embodiments, a recombinant Cas effector protein is fused to a protein or a fragment of a protein that binds DNA molecules or bind other cellular molecules, including but not limited to: maltose binding protein (MBP), S-tag, Lex A DNA binding domain (DBD), GAL4 DNA binding domain, and herpes simplex virus (HSV) BP16 protein. Additional domains that may form part of a fusion protein including a Cas effector protein are described in U.S. Patent Publication 2011/0059502. In some embodiments, a tagged recombinant Cas effector protein is used to identify the location of a target sequence.

In some embodiments, the Cas effector protein is fused to a heterologous protein or protein domain. In some embodiments, the Cas effector protein is fused to a reverse transcriptase. In some embodiments, the Cas effector protein is a Cas9 nuclease fused to a reverse transcriptase.

Examples of such Cas9-reverse transcriptase fusions are described in Anzalone et al., Nature, 576:149-157 (2019).

In some embodiments, the Cas effector protein is fused to a DNA polymerase. In some embodiments, the Cas effector protein is a Cas9 nuclease fused to a DNA polymerase.

In some embodiments, the Cas effector protein is fused to a dominant negative 53BP1 (also known as TP53BP1, tumor suppressor p53-binding protein 1). In some embodiments, the Cas effector protein is a Cas9 nuclease fused to a dominant negative 53BP1 protein. In some embodiments, the dominant negative 53BP1 protein is DNlS. In some embodiments, the Cas effector protein is a Cas9 nuclease fused to DNlS.

In some embodiments, the Cas effector protein is fused to a Geminin degron domain. IN some embodiments, the Cas effector protein is a Cas9 nuclease fused to a Geminin degron domain. Examples of such proteins are described in Gutschner et al, Cell Reports, 14:1555-1566 (2016).

In some embodiments, the Cas effector protein is fused to a CtIP (C-terminal binding protein 1) protein. In some embodiments, the Cas effector protein is a Cas9 nuclease fused to a CtIP protein.

In some embodiments, a recombinant Cas effector protein may form a component of an inducible system. The inducible nature of the system allows for spatiotemporal control of gene editing or gene expression using a form of energy. The form of energy can include, but is not limited to: electromagnetic radiation, sound energy, chemical energy, and thermal energy. Non-limiting examples of inducible system include: tetracycline inducible promoters (Tet-On or Tet-Off), small molecule two-hybrid transcription activations systems (FKBP, ABA, etc), or light inducible systems (Phytochrome, LOV domains, or cryptochrome). In some embodiments, the Cas effector protein is a part of a Light Inducible Transcriptional Effector (LITE) to direct changes in transcriptional activity in a sequence-specific manner. The components of a light may include a Cas effector protein, a light-responsive cytochrome heterodimer (e.g., from Arabidopsis thaliana), and a transcriptional activation/repression domain. Further examples of inducible DNA binding proteins and methods for their use are provided in International Application Publication Nos. WO 2014/018423 and WO 2014/093635; U.S. Pat. Nos. 8,889,418 and 8,895,308; and U.S. Patent Publication Nos. 2014/0186919, 2014/0242700, 2014/0273234, and 2014/0335620.

TABLE 1

| | |
|---|---|
| SEQ ID NO: 1 | MLFNKCIIISINLDFSNKEKCMTKPYSIGL |
| | DIGTNSVGWAVITDNYKVPSKKMKVLGNTS |
| | KKYIKKNLLGVLLFDSGITAEGRRLKRTAR |
| | RRYTRRRNRILYLQEIFSTEMATLDDAFFQ |
| | RLDDSFLVPDDKRDSKYPIFGNLVEEKVYH |
| | DEFPTIYHLRKYLADSTKKADLRLVYLALA |
| | HMIKYRGHFLIEGEFNSKNNDIQKNFQDFL |
| | DTYNAIFESDLSLENSKQLEEIVKDKISKL |
| | EKKDRILKLFPGEKNSGIFSEFLKLIVGNQ |
| | ADFRKCFNLDEKASLHESKESYDEDLETLL |
| | GYIGDDYSDVFLKAKKLYDAILLSGFLTVT |
| | DNETEAPLSSAMIKRYNEHKEDLALLKEYI |
| | RNISLKTYNEVFKDDTKNGYAGYIDGKTNQ |
| | EDFYVYLKNLLAEFEGADYFLEKIDREDFL |
| | RKQRTFDNGSIPYQIHLQEMRAILDKQAKF |
| | YPFLAKNKERIEKILTFRIPYYVGPLARGN |
| | SDFAWSIRKRNEKITPWNFEDVIDKESSAE |
| | AFINRMTSFDLYLPEEKVLPKHSLLYETFN |
| | VYNELTKVRFIAESMRDYQFLDSKQKKDIV |
| | RLYFKDKRKVTDKDIIEYLHAIYGYDGIEL |
| | KGIEKQFNSSLSTYHDLLNIINDKEFLDDS |
| | SNEAIIEEIIHTLTIFEDREMIKQRLSKFE |
| | NIFDKSVLKKLSRRHYTGWGKLSAKLINGI |
| | RDEKSGNTILDYLIDDGISNRNFMQLIHDD |
| | ALSFKKKIQKAQIIGDEDKGNIKEVVKSLP |
| | GSPAIKKGILQSIKIVDELVKVMGGRKPES |
| | IVVEMARENQYTNQGKSNSQQRLKRLEKSL |
| | KELGSKILKENIPAKLSKIDNNALQNDRLY |
| | LYYLQNGKDMYTGDDLDIDRLSNYDIDHII |
| | PQAFLKDNSIDNKVLVSSASNRGKSDDFPS |
| | LEVVKKRKTFWYQLLKSKLISQRKFDNLTK |
| | AERGGLLPEDKAGFIQRQLVETRQITKHVA |
| | RLLDEKFNNKKDENNRAVRTVKIITLKSTL |
| | VSQFRKDFELYKVREINDFHHAHDAYLNAV |
| | IASALLKKYPKLEPEFVYGDYPKYNSFRER |
| | KSATEKVYFYSNIMNIFKKSISLADGRVIE |
| | RPLIEVNEETGESVWNKESDLATVRRVLSY |
| | PQVNVVKKVEEQNHGLDRGKPKGLFNANLS |
| | SKPKPNSNENLVGAKEYLDPKKYGGYAGIS |
| | NSFAVLVKGTIEKGAKKKITNVLEFQGISI |
| | LDRINYRKDKLNFLLEKGYKDIELIIELPK |
| | YSLFELSDGSRRMLASILSTNNKRGEIHKG |
| | NQIFLSQKFVKLLYHAKRISNTINENHRKY |
| | VENHKKEFEELFYYILEFNENYVGAKKNGK |
| | LLNSAFQSWQNHSIDELCSSFIGPTGSERK |
| | GLFELTSRGSAADFEFLGVKIPRYRDYTPS |
| | SLLKDATLIHQSVTGLYETRIDLAKLGEG |
| SEQ ID NO: 2 | MDKKYSIGLDIGTNSVGWAVITDEYKVPSK |
| | KFKVLGNTDRHSIKKNLIGALLFDSGETAE |
| | ATRLKRTARRRYTRRKNRICYLQEIFSNEM |
| | AKVDDSFFHRLEESFLVEEDKKHERHPIFG |
| | NIVDEVAYHEKYPTIYHLRKKLVDSTDKAD |
| | LRLIYLALAHMIKFRGHFLIEGDLNPDNSD |
| | VDKLFIQLVQTYNQLFEENPINASGVDAKA |
| | ILSARLSKSRRLENLIAQLPGEKKNGLFGN |
| | LIALSLGLTPNFKSNFDLAEDAKLQLSKDT |
| | YDDDLDNLLAQIGDQYADLFLAAKNLSDAI |
| | LLSDILRVNTEITKAPLSASMIKRYDEHHQ |
| | DLTLLKALVRQQLPEKYKEIFFDQSKNGYA |
| | GYIDGGASQEEFYKFIKPILEKMDGTEELL |
| | VKLNREDLLRKQRTFDNGSIPHQIHLGELH |
| | AILRRQEDFYPFLKDNREKIEKILTFRIPY |
| | YVGPLARGNSRFAWMTRKSEETITPWNFEE |
| | VVDKGASAQSFIERMTNFDKNLPNEKVLPK |
| | HSLLYEYFTVYNELTKVKYVTEGMRKPAFL |
| | SGEQKKAIVDLLFKTNRKVTVKQLKEDYFK |
| | KIECFDSVEISGVEDRFNASLGTYHDLLKI |
| | IKDKDFLDNEENEDILEDIVLTLTLFEDRE |
| | MIEERLKTYAHLFDDKVMKQLKRRRYTGWG |
| | RLSRKLINGIRDKQSGKTILDFLKSDGFAN |
| | RNFMQLIHDDSLTFKEDIQKAQVSGQGDSL |
| | HEHIANLAGSPAIKKGILQTVKVVDELVKV |
| | MGRHKPENIVIEMARENQTTQKGQKNSRER |
| | MKRIEEGIKELGSQILKEHPVENTQLQNEK |
| | LYLYYLQNGRDMYVQELDINRLSDYDVDHI |
| | VPQSFLKDDSIDNKVLTRSDKNRGKSDNVP |
| | SEEVVKKMKNYWRQLLNAKLITQRKFDNLT |
| | KAERGGLSELDKAGFIKRQLVETRQITKHV |
| | AQILDSRMNTKYDENDKLIREVKVITLKSK |
| | LVSDFRKDFQFYKVREINNYHHAHDAYLNA |
| | VVGTALIKKYPKLESEFVYGDYKVYDVRKM |
| | IAKSEQEIGKATAKYFFYSNIMNFFKTEIT |
| | LANGEIRKRPLIETNGETGEIVWDKGRDFA |
| | TVRKVLSMPQVNIVKKTEVQTGGFSKESIL |
| | PKRNSDKLIARKKDWDPKKYGGFDSPTVAY |
| | SVLVVAKVEKGKSKKLKSVKELLGITIMER |
| | SSFEKNPIDFLEAKGYKEVKKDLIIKLPKY |
| | SLFELENGRKRMLASAGELQKGNELALPSK |
| | YVNFLYLASHYEKLKGSPEDNEQKQLFVEQ |
| | HKHYLDEIIEQISEFSKRVILADANLDKVL |
| | SAYNKHRDKPIREQAENIIHLFTLTNLGAP |
| | AAFKYFDTTIDRKRYTSTKEVLDATLIHQS |
| | ITGLYETRIDLSQLGGD |
| SEQ ID NO: 3 | MKRNYILGLDIGITSVGYGIIDYETRDVID |
| | AGVRLFKEANVENNEGRRSKRGARRLKRRR |
| | RHRIQRVKKLLFDYNLLTDHSELSGINPYE |
| | ARVKGLSQKLSEEEFSAALLHLAKRRGVHN |
| | VNEVEEDTGNELSTKEQISRNSKALEEKYV |
| | AELQLERLKKDGEVRGSINRFKTSDYVKEA |
| | KQLLKVQKAYHQLDQSFIDTYIDLLETRRT |
| | YYEGPGEGSPFGWKDIKEWYEMLMGHCTYF |
| | PEELRSVKYAYNADLYNALNDLNNLVITRD |
| | ENEKLEYYEKFQIIENVFKQKKKPTLKQIA |
| | KEILVNEEDIKGYRVTSTGKPEFTNLKVYH |
| | DIKDITARKEIIENAELLDQIAKILTIYQS |
| | SEDIQEELTNLNSELTQEEIEQISNLKGYT |
| | GTHNLSLKAINLILDELWHTNDNQIAIFNR |
| | LKLVPKKVDLSQQKEIPTTLVDDFILSPVV |
| | KRSFIQSIKVINAIIKKYGLPNDIIIELAR |
| | EKNSKDAQKMINEMQKRNRQTNERIEEIIR |
| | TTGKENAKYLIEKIKLHDMQEGKCLYSLEA |
| | IPLEDLLNNPFNYEVDHIIPRSVSFDNSFN |
| | NKVLVKQEENSKKGNRTPFQYLSSSDSKIS |
| | YETFKKHILNLAKGKGRISKTKKEYLLEER |
| | DINRFSVQKDFINRNLVDTRYATRGLMNLL |
| | RSYFRVNNLDVKVKSINGGFTSFLRRKWKF |
| | KKERNKGYKHHAEDALIIANADFIFKEWKK |
| | LDKAKKVMENQMFEEKQAESMPEIETEQEY |
| | KEIFITPHQIKHIKDFKDYKYSHRVDKKPN |
| | RELINDTLYSTRKDDKGNTLIVNNLNGLYD |
| | KDNDKLKKLINKSPEKLLMYHHDPQTYQKL |
| | KLIMEQYGDEKNPLYKYYEETGNYLTKYSK |
| | KDNGPVIKKIKYYGNKLNAHLDITDDYPNS |
| | RNKVVKLSLKPYRFDVYLDNGVYKFVTVKN |
| | LDVIKKENYYEVNSKCYEEAKKLKKISNQA |
| | EFIASFYNNDLIKINGELYRVIGVNNDLLN |
| | RIEVNMIDITYREYLENMNDKRPPRIIKTI |
| | ASKTQSIKKYSTDILGNLYEVKSKKHPQII |
| | KKG |
| SEQ ID NO: 4 | MSIYQEFVNKYSLSKTLRFELIPQGKTLEN |
| | IKARGLILDDEKRAKDYKKAKQIIDKYHQF |
| | FIEEILSSVCISEDLLQNYSDVYFKLKKSD |
| | DDNLQKDFKSAKDTIKKQISEYIKDSEKFK |
| | NLFNQNLIDAKKGQESDLILWLKQSKDNGI |
| | ELFKANSDITDIDEALEIIKSFKGWTTYFK |
| | GFHENRKNVYSSNDIPTSIIYRIVDDNLPK |
| | FLENKAKYESLKDKAPEAINYEQIKKDLAE |
| | ELTFDIDYKTSEVNQRVFSLDEVFEIANFN |
| | NYLNQSGITKENTIIGGKFVNGENTKRKGI |
| | NEYINLYSQQINDKTLKKYKMSVLFKQILS |
| | DTESKSFVIDKLEDDSDVVTTMQSFYEQIA |
| | AFKTVEEKSIKETLSLLFDDLKAQKLDLSK |
| | IYFKNDKSLTDLSQQVEDDYSVIGTAVLEY |
| | ITQQIAPKNLDNPSKKEQELIAKKTEKAKY |
| | LSLETIKLALEEFNKHRDIDKQCRFEEILA |
| | NFAAIPMIFDEIAQNKDNLAQISIKYQNQG |
| | KKDLLQASAEDDVKAIKDLLDQTNNLLHKL |
| | KIFHISQSEDKANILDKDEHFYLVFEECYF |
| | ELANIVPLYNKIRNYITQKPYSDEKFKLNF |
| | ENSTLANGWDKNKEPDNTAILFIKDDKYYL |
| | GVMNKKNNKIFDDKAIKENKGEGYKKIVYK |
| | LLPGANKMLPKVFFSAKSIKFYNPSEDILR |
| | IRNHSTHTKNGSPQKGYEKFEFNIEDCRKF |
| | IDFYKQSISKHPEWKDFGFRFSDTQRYNSI |
| | DEFYREVENQGYKLTFENISESYIDSVVNQ |
| | GKLYLFQIYNKDFSAYSKGRPNLHTLYWKA |
| | LFDERNLQDVVYKLNGEAELFYRKQSIPKK |
| | ITHPAKEAIANKNKDNPKKESVFEYDLIKD |
| | KRFTEDKFFFHCPITINFKSSGANKFNDEI |
| | NLLLKEKANDVHILSIDRGERHLAYYTLVD |
| | GKGNIIKQDTFNIIGNDRMKTNYHDKLAAI |
| | EKDRDSARKDWKKINNIKEMKEGYLSQVVH |
| | EIAKLVIEYNAIVVFEDLNFGFKRGRFKVE |
| | KQVYQKLEKMLIEKLNYLVFKDNEFDKTGG |
| | VLRAYQLTAPFETFKKMGKQTGIIYYVPAG |
| | FTSKICPVTGFVNQLYPKYESVSKSQEFFS |
| | KFDKICYNLDKGYFEFSFDYKNFGDKAAKG |
| | KWTIASFGSRLINFRNSDKNHNWDTREVYP |
| | TKELEKLLKDYSIEYGHGECIKAAICGESD |
| | KKFFAKLTSVLNTILQMRNSKTGTELDYLI |
| | SPVADVNGNFFDSRQAPKNMPQDADANGAY |
| | HIGLKGLMLLGRIKNNQEGKKLNLVIKNEE |
| | YFEFVQNRNN |
| SEQ ID NO: 5 | MTQFEGFTNLYQVSKTLRFELIPQGKTLKH |
| | IQEQGFIEEDKARNDHYKELKPIIDRIYKT |
| | YADQCLQLVQLDWENLSAAIDSYRKEKTEE |
| | TRNALIEEQATYRNAIHDYFIGRTDNLTDA |
| | INKRHAEIYKGLFKAELFNGKVLKQLGTVT |
| | TTEHENALLRSFDKFTTYFSGFYENRKNVF |
| | SAEDISTAIPHRIVQDNFPKFKENCHIFTR |
| | LITAVPSLREHFENVKKAIGIFVSTSIEEV |
| | FSFPFYNQLLTQTQIDLYNQLLGGISREAG |
| | TEKIKGLNEVLNLAIQKNDETAHIIASLPH |
| | RFIPLFKQILSDRNTLSFILEEFKSDEEVI |
| | QSFCKYKTLLRNENVLETAEALFNELNSID |
| | LTHIFISHKKLETISSALCDHWDTLRNALY |
| | ERRISELTGKITKSAKEKVQRSLKHEDINL |
| | QEIISAAGKELSEAFKQKTSEILSHAHAAL |
| | DQPLPTTLKKQEEKEILKSQLDSLLGLYHL |
| | LDWFAVDESNEVDPEFSARLTGIKLEMEPS |
| | LSFYNKARNYATKKPYSVEKFKLNFQMPTL |
| | ASGWDVNKEKNNGAILFVKNGLYYLGIMPK |
| | QKGRYKALSFEPTEKTSEGFDKMYYDYFPD |
| | AAKMIPKCSTQLKAVTAHFQTHTTPILLSN |
| | NFIEPLEITKEIYDLNNPEKEPKKFQTAYA |
| | KKTGDQKGYREALCKWIDFTRDFLSKYTKT |
| | TSIDLSSLRPSSQYKDLGEYYAELNPLLYH |
| | ISFQRIAEKEIMDAVETGKLYLFQIYNKDF |
| | AKGHHGKPNLHTLYWTGLFSPENLAKTSIK |
| | LNGQAELFYRPKSRMKRMAHRLGEKMLNKK |
| | LKDQKTPIPDTLYQELYDYVNHRLSHDLSD |
| | EARALLPNVITKEVSHEIIKDRRFTSDKFF |
| | FHVPITLNYQAANSPSKFNQRVNAYLKEHP |
| | ETPIIGIDRGERNLIYITVIDSTGKILEQR |
| | SLNTIQQFDYQKKLDNREKERVAARQAWSV |
| | VGTIKDLKQGYLSQVIHEIVDLMIHYQAVV |
| | VLENLNFGFKSKRTGIAEKAVYQQFEKMLI |
| | DKLNCLVLKDYPAEKVGGVLNPYQLTDQFT |
| | SFAKMGTQSGFLFYVPAPYTSKIDPLTGFV |
| | DPFVWKTIKNHESRKHFLEGFDFLHYDVKT |
| | GDFILHFKMNRNLSFQRGLPGFMPAWDIVF |
| | EKNETQFDAKGTPFIAGKRIVPVIENHRFT |
| | GRYRDLYPANELIALLEEKGIVFRDGSNIL |
| | PKLLENDDSHAIDTMVALIRSVLQMRNSNA |
| | ATGEDYINSPVRDLNGVCFDSRFQNPEWPM |
| | DADANGAYHIALKGQLLLNHLKESKDLKLQ |
| | NGISNQDWLAYIQELRN |
| SEQ ID NO: 6 | MAVKSIKVKLRLDDMPEIRAGLWKLHKEVN |
| | AGVRYYTEWLSLLRQENLYRRSPNGDGEQE |
| | CDKTAEECKAELLERLRARQVENGHRGPAG |
| | SDDELLQLARQLYELLVPQAIGAKGDAQQI |
| | ARKFLSPLADKDAVGGLGIAKAGNKPRWVR |
| | MREAGEPGWEEEKEKAETRKSADRTADVLR |
| | ALADFGLKPLMRVYTDSEMSSVEWKPLRKG |
| | QAVRTWDRDMFQQAIERMMSWESWNQRVGQ |
| | EYAKLVEQKNRFEQKNFVGQEHLVHLVNQL |
| | QQDMKEASPGLESKEQTAHYVTGRALRGSD |
| | KVFEKWGKLAPDAPFDLYDAEIKNVQRRNT |
| | RRFGSHDLFAKLAEPEYQALWREDASFLTR |
| | YAVYNSILRKLNHAKMFATFTLPDATAHPI |
| | WTRFDKLGGNLHQYTFLFNEFGERRHAIRF |
| | HKLLKVENGVAREVDDVTVPISMSEQLDNL |
| | LPRDPNEPIALYFRDYGAEQHFTGEFGGAK |
| | IQCRRDQLAHMHRRRGARDVYLNVSVRVQS |
| | QSEARGERRPPYAAVFRLVGDNHRAFVHFD |
| | KLSDYLAEHPDDGKLGSEGLLSGLRVMSVD |
| | LGLRTSASISVFRVARKDELKPNSKGRVPF |
| | FFPIKGNDNLVAVHERSQLLKLPGETESKD |
| | LRAIREERQRTLRQLRTQLAYLRLLVRCGS |
| | EDVGRRERSWAKLIEQPVDAANHMTPDWRE |
| | AFENELQKLKSLHGICSDKEWMDAVYESVR |
| | RVWRHMGKQVRDWRKDVRSGERPKIRGYAK |
| | DVVGGNSIEQIEYLERQYKFLKSWSFFGKV |
| | SGQVIRAEKGSRFAITLREHIDHAKEDRLK |
| | KLADRIIMEALGYVYALDERGKGKWVAKYP |
| | PCQLILLEELSEYQFNNDRPPSENNQLMQW |
| | SHRGVFQELINQAQVHDLLVGTMYAAFSSR |
| | FDARTGAPGIRCRRVPARCTQEHNPEPFPW |
| | WLNKFVVEHTLDACPLRADDLIPTGEGEIF |
| | VSPFSAEEGDFHQIHADLNAAQNLQQRLWS |
| | DFDISQIRLRCDWGEVDGELVLIPRLTGKR |
| | TADSYSNKVFYTNTGVTYYERERGKKRRKV |
| | FAQEKLSEEEAELLVEADEAREKSVVLMRD |
| | PSGIINRGNWTRQKEFWSMVNQRIEGYLVK |
| | QIRSRVPLQDSACENTGDI |
| SEQ ID NO: 7 | YGRKKRRQRRR |
| SEQ ID NO: 8 | RRQRRTSKLMKR |
| SEQ ID NO: 9 | GWTLNSAGYLLGKINLKALAALAKKIL |
| SEQ ID NO: 10 | KALAWEAKLAKALAKALAKHLAKALAKALK |
| | CEA |
| SEQ ID NO: 11 | RQIKIWFQNRRMKWKK |
| SEQ ID NO: 12 | YGRKKRRQRRR |
| SEQ ID NO: 13 | RKKRRQRRR |
| SEQ ID NO: 14 | YGRKKRRQRRR |
| SEQ ID NO: 15 | RKKRRQRR |
| SEQ ID NO: 16 | YARAAARQARA |
| SEQ ID NO: 17 | THRLPRRRRRR |
| SEQ ID NO: 18 | GGRRARRRRRR |

### Nucleotides

i. Sequence of Interest

In some embodiments, a polynucleotide of the disclosure is an exogenous polynucleotide which comprises a sequence of interest (SOI) to be inserted into the genome of a eukaryotic cell. In some embodiments, the sequence of interest encodes a gene of interest.

In some embodiments, the polynucleotide comprising exogenous polynucleotide comprising a SOI is an exogenous polynucleotide template which is inserted into the genome of a eukaryotic cell via CRISPR/Cas-mediated homologous recombination. In some embodiments, the SOI comprises at least one mutation of interest to be inserted into a genome of a eukaryotic cell. In some embodiments, the SOI comprises a gene of interest to be inserted into a genome of a eukaryotic cell. In some embodiments, the SOI can be introduced as an exogenous polynucleotide template. In some embodiments, the SOI is a hybrid polynucleotide comprising single-stranded and double-stranded regions. In some embodiments, the hybrid polynucleotide comprises double-stranded sequences at the 5′ and 3′ ends and an internal single-stranded sequence (Shy et al, bioRxiv, 2021, preprint published Sep. 2, 2021). In some embodiments, the exogenous polynucleotide includes blunt ends. In some embodiments, the exogenous polynucleotide template includes cohesive ends. In some embodiments, the exogenous polynucleotide template includes cohesive ends complementary to cohesive ends in the target sequence.

The exogenous polynucleotide template can be of any suitable length, such as about or at least about 10, 15, 20, 25, 50, 75, 100, 150, 200, 250, 500, 1000, 5000, or 10,000 or more nucleotides in length. In some embodiments, the exogenous polynucleotide template is complementary to a portion of a polynucleotide including the target sequence. In some embodiments, when optimally aligned, the exogenous polynucleotide template overlaps with one or more nucleotides of a target sequence (e.g., about or at least about 1, 5, 10, 15, 20, 25, 30, 35, 40, 45, 50, 60, 70, 80, 90, or 100 or more nucleotides). In some embodiments, when the exogenous polynucleotide template and a polynucleotide including the target sequence are optimally aligned, the nearest nucleotide of the exogenous polynucleotide template is within about 1, 5, 10, 15, 20, 25, 50, 75, 100, 200, 300, 400, 500, 100, 1500, 2000, 2500, 5000, 10,000 or more nucleotides from the target sequence.

In some embodiments, the exogenous polynucleotide is DNA, such as, e.g., a DNA plasmid, a bacterial artificial chromosome (BAC), a yeast artificial chromosome (YAC), a viral vector, a linear piece of single-stranded or double-stranded DNA, an oligonucleotide, a PCR fragment, a naked nucleic acid, or a nucleic acid complexed with a delivery vehicle such as a liposome. In some embodiments, the exogenous polynucleotide is RNA. In some embodiments, the RNA is a messenger RNA (mRNA).

In some embodiments, the exogenous polynucleotide is inserted into the target sequence using an endogenous DNA repair pathway of the cell. In some embodiments, the endogenous DNA repair pathway is HDR. During the repair process, an exogenous polynucleotide template including the SOI can be introduced into the target sequence. In some embodiments, an exogenous polynucleotide template including the SOI flanked by an upstream sequence and a downstream sequence is introduced into the cell, where the upstream and downstream sequences share sequence similarity with either side of the site of integration in the target sequence. In some embodiments, the exogenous polynucleotide including the SOI includes, for example, a mutated gene. In some embodiments, the exogenous polynucleotide includes a sequence endogenous or exogenous to the cell. In some embodiments, the SOI includes polynucleotides encoding a protein, or a non-coding sequence such as, e.g., a microRNA. In some embodiments, the SOI is operably linked to a regulatory element. In some embodiments, the SOI is a regulatory element. In some embodiments, the SOI includes a resistance cassette, e.g., a gene that confers resistance to an antibiotic. In some embodiments, the SOI includes a mutation of the wild-type target sequence. In some embodiments, the SOI disrupts or corrects the target sequence by creating a frameshift mutation or nucleotide substitution. In some embodiments, the SOI includes a marker. Introduction of a marker into a target sequence can make it easy to screen for targeted integrations. In some embodiments, the marker is a restriction site, a fluorescent protein, or a selectable marker. In some embodiments, the SOI is introduced as a vector including the SOI.

The upstream and downstream sequences in the exogenous polynucleotide template are selected to promote homologous recombination between the target sequence and the exogenous polynucleotide. The upstream sequence is a nucleic acid sequence that shares sequence similarity with the sequence upstream of the targeted site for integration (i.e., the target sequence). Similarly, the downstream sequence is a nucleic acid sequence that shares sequence similarity with the sequence downstream of the targeted site for integration. Thus, in some embodiments, the exogenous polynucleotide template including the SOI is inserted into the target sequence by homologous recombination at the upstream and downstream sequences. In some embodiments, the upstream and downstream sequences in the exogenous polynucleotide template have at least 70%, at least 75%, at least 80%, at least 85%, at least 90%, at least 95%, at least 96%, at least 97%, at least 98%, at least 99%, or 100% sequence identity with the upstream and downstream sequences of the targeted genome sequence, respectively. In some embodiments, the upstream or downstream sequence has at least about 20, 50, 100, 150, 200, 250, 300, 350, 400, or 500 base pairs and up to about 600, 750, 1000, 1250, 1500, 1750 or 2000 base pairs. In some embodiments, the upstream or downstream sequence has about 20 to 2000 base pairs, or about 50 to 1750 base pairs, or about 100 to 1500 base pairs, or about 200 to 1250 base pairs, or about 300 to 1000 base pairs, or about 400 to about 750 base pairs, or about 500 to 600 base pairs. In some embodiments, the upstream or downstream sequence has about 50, about 100, about 250, about 500, about 100, about 1250, about 1500, about 1750, about 2000, about 2250, or about 2500 base pairs.

In some embodiments, the SOI comprises a gene of interest. As used herein, the term “gene of interest” refers to a gene that encodes a biomolecule of interest (e.g., a protein or an RNA molecule). In some embodiments, the gene of interest encodes a protein of interest. In some embodiments, the protein of interest comprises an intracellular protein, a membrane protein, an extracellular protein, or combination thereof. In some embodiments, the protein of interest comprises a nuclear protein, a transcription factor, a nuclear membrane transporter, an intracellular organelle associated protein, a membrane receptor, a catalytic protein, an enzyme, a therapeutic protein, a membrane protein, a membrane transport protein, a signal transduction protein, an immunological protein, or combination thereof. In some embodiments, the immunological protein comprises an antibody, e.g., IgG, IgA, IgM, IgD, IgE, or combination thereof. In some embodiments, the immunological protein is a T cell receptor (TCR). In some embodiments, immunological protein is a chimeric antigen receptor (CAR). In some embodiments, the SOI encodes a copy of a native gene of the host cell. In some embodiments, the SOI encodes a copy of a native gene that is deficient in the host cell. In some embodiments, the host cell comprises a mutation in a gene, and the SOI encodes a wild-type copy of the gene. In some embodiments, the host cell comprises a wild-type gene, and the SOI encodes a copy of the gene comprising a mutation of interest. In some embodiments, the SOI encodes a heterologous gene that is not naturally occurring in the host cell.

In some embodiments, the gene of interest encodes an RNA of interest. In some embodiments, the RNA of interest comprises a therapeutic RNA. In some embodiments, the RNA of interest comprises messenger RNA (mRNA), transfer RNA (tRNA), ribosomal RNA (rRNA), small nuclear RNA (snRNA), antisense RNA, microRNA (miRNA), small interfering RNA (siRNA), cell-free RNA (cfRNA), or combination thereof. In some embodiments, the sequence of interest comprises a regulatory element of interest. In some embodiments, the SOI is inserted into a target polynucleotide of a host cell, such that the regulatory element on the sequence of interest is capable of regulating a native gene of the host cell. Regulatory elements are described herein and include, e.g., promoters, enhancers, silencers, operators, response elements, 5′ UTR, 3′ UTR, insulators, and the like.

In some embodiments, the polynucleotide comprising a SOI is about 1 nucleotide to about 5000 nucleotides in length. In some embodiments, the polynucleotide comprising the SOI is about 5 nucleotides to about 5000 nucleotides in length. In some embodiments, polynucleotide comprising a SOI is about 6 nucleotides to about 1000 nucleotides in length. In some embodiments, the polynucleotide comprising a SOI is about 7 nucleotides to about 750 nucleotides in length. In some embodiments, the polynucleotide comprising a SOI is about 8 nucleotides to about 500 nucleotides in length. In some embodiments, the polynucleotide comprising a SOI is about 9 nucleotides to about 250 nucleotides in length. In some embodiments, the polynucleotide comprising a SOI is about 10 nucleotides to about 100 nucleotides in length. In some embodiments, the polynucleotide comprising a SOI is about 15 nucleotides to about 90 nucleotides in length. In some embodiments, the polynucleotide comprising a SOI is about 20 nucleotides to about 80 nucleotides in length. In some embodiments, the polynucleotide comprising a SOI is about 25 nucleotides to about 70 nucleotides in length. In some embodiments, the polynucleotide comprising a SOI is about 30 nucleotides to about 50 nucleotides in length. In some embodiments, the polynucleotide comprising a SOI is about 1 to about 10 nucleotides in length. In some embodiments, the polynucleotide comprising a SOI is about 1 to about 20 nucleotides in length. In some embodiments, the polynucleotide comprising a SOI is about 1 to about 30 nucleotides in length. In some embodiments, the polynucleotide comprising a SOI is about 10 to about 40 nucleotides in length. In some embodiments, the polynucleotide comprising a SOI is about 1 to about 50 nucleotides in length. In some embodiments, the polynucleotide comprising a SOI is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the polynucleotide comprising a SOI is greater than about 10 nucleotides, greater than about 15 nucleotides, greater than about 20 nucleotides, greater than about 25 nucleotides, greater than about 30 nucleotides, greater than about 35 nucleotides, greater than about 40 nucleotides, greater than about 45 nucleotides, or greater than about 50 nucleotides in length.

In some embodiments, the SOI is about 3 to about 5000 nucleotides in length. In some embodiments, the SOI is about 4 to about 1000 nucleotides in length. In some embodiments, the SOI is about 5 to about 900 nucleotides in length. In some embodiments, the SOI is about 6 to about 800 nucleotides in length. In some embodiments, the SOI is about 7 to about 700 nucleotides in length. In some embodiments, the SOI is about 8 to about 600 nucleotides in length. In some embodiments, the SOI is about 9 to about 500 nucleotides in length. In some embodiments, the SOI is about 50 to about 5000 nucleotides in length. In some embodiments, the SOI is about 60 to about 1000 nucleotides in length. In some embodiments, the SOI is about 70 to about 900 nucleotides in length. In some embodiments, the SOI is about 8 to about 800 nucleotides in length. In some embodiments, the SOI is about 90 to about 700 nucleotides in length. In some embodiments, the SOI is about 100 to about 500 nucleotides in length. In some embodiments, the SOI is about 100 to about 250 nucleotides in length. In some embodiments, the SOI is about 10 to about 90 nucleotides in length. In some embodiments, the SOI is about 11 to about 80 nucleotides in length. In some embodiments, the SOI is about 12 to about 70 nucleotides in length. In some embodiments, the SOI is about 15 to about 60 nucleotides in length. In some embodiments, the SOI is about 10 to about 50 nucleotides in length. In some embodiments, the SOI is about 1 to about 10 nucleotides in length. In some embodiments, the SOI is about 1 to about 25 nucleotides in length. In some embodiments, the SOI is about 1 to about 50 nucleotides in length. In some embodiments, the SOI is about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 nucleotides in length. In some embodiments, the SOI is greater than about 10 nucleotides, greater than about 15 nucleotides, greater than about 20 nucleotides, greater than about 25 nucleotides, greater than about 30 nucleotides, greater than about 35 nucleotides, greater than about 40 nucleotides, greater than about 45 nucleotides, or greater than about 50 nucleotides in length.

ii. Cas and Cas-Associated Polynucleotides

In some embodiments, the present disclosure encompasses nucleotide or polynucleotide sequences which encode a Cas effector protein of the disclosure, i.e., a Cas polynucleotide.

In some embodiments, a polynucleotide of the disclosure is capable of forming a complex with a Cas effector protein. In some embodiments, the polynucleotide capable of forming a complex with a Cas effector protein comprise a guide sequence. In some embodiments, the polynucleotide capable of forming a complex with a Cas effector protein comprises a Cas-binding region. In some embodiments, the polynucleotide capable of forming a complex with a Cas effector protein comprises a DNA template sequence. In some embodiments, the polynucleotide capable of forming a complex with a Cas effector protein comprises a guide sequence, a Cas-binding region, and a DNA template sequence, or any combination thereof. In some embodiments, the polynucleotide comprises, in 5′ to 3′ order, a guide sequence, a Cas-binding region, and a DNA template sequence.

In some embodiments, the guide sequence is capable of hybridizing with a target polynucleotide, e.g., a target polynucleotide in a genome of a host cell. In embodiments, the guide sequence is complementary to the target polynucleotide. In some embodiments, the target polynucleotide is a target DNA intended to be cleaved by the Cas nuclease or Cas nickase. In some embodiments, the guide sequence comprises RNA, i.e., an RNA guide sequence. In some embodiments, the guide sequence comprises a combination of RNA and DNA. Hybrid RNA-DNA guide sequences are further described in, e.g., Rueda et al., Nat Comm 8:1610 (2017).

In some embodiments, the guide sequence is about 10 to about 40 nucleotides in length. In some embodiments, the guide sequence is about 12 to about 30 nucleotides in length. In some embodiments, the guide sequence is about 15 to about 20 nucleotides in length. In some embodiments, the guide sequence is about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 nucleotides in length. In some embodiments, the guide sequence is a sufficient length for hybridizing to the target polynucleotide.

In some embodiments, the Cas-binding region is capable of binding to the Cas effector protein (e.g., Cas nuclease or Cas nickase), thereby forming a complex with the Cas protein. In some embodiments, the Cas-binding region comprises RNA. In some embodiments, the Cas-binding region comprises a combination of RNA and DNA. Hybrid RNA-DNA sequences that can bind to and/or activate Cas proteins are further described in, e.g., Rueda et al., Nat Comm 8:1610 (2017).

In some embodiments, multiple guide RNA as described in the methods, kits, and compositions described herein can be used during the same method, kit or composition. For example, in some embodiments, 2, 3, 4, 5, 6, 7, 8, 9 or 10 or more different guide RNA can be used at the same time.

In some embodiments, the Cas-binding region comprises a tracrRNA that binds to and activates the Cas protein. In some embodiments, the Cas-binding region is capable of hybridizing with a tracrRNA, and the composition further comprises a tracrRNA. In some embodiments, the tracrRNA is capable of binding the Cas nuclease or Cas nickase. In some embodiments, the tracrRNA is capable of activating the Cas nuclease or Cas nickase. In some embodiments, the activating comprises initiating or increasing the cleavage activity of the Cas nuclease or Cas nickase. In some embodiments, the activating comprises promoting binding of the Cas nuclease or Cas nickase to a target polynucleotide (e.g., as guided by the guide sequence). In some embodiments, the activating comprises a combination of promoting binding of the Cas nuclease or Cas nickase to the target polynucleotide; and initiating or increasing cleavage activity of the Cas nuclease or Cas nickase. TracrRNA sequences of Cas proteins (e.g., Cas9, Cas12a, or Type II-B Cas proteins described herein) are available from public databases, including RNAcentral and Rfam, and further described in, e.g., Chylinski et al., RNA Biol 10(5):726-737 (2013) and Gasiunas et al., Nat Comm 11:5512 (2020).

In some embodiments, the polynucleotide capable of forming a complex with a Cas effector molecule comprises a DNA template sequence at a 3′ end of the polynucleotide. In some embodiments, the DNA template sequence comprises single-stranded DNA. In some embodiments, the DNA template sequence comprises a sequence of interest. In some embodiments, the DNA template sequence comprises a primer binding sequence and a sequence of interest. In some embodiments, the DNA template sequence comprises a template for amplification by a DNA polymerase. In some embodiments, the sequence of interest comprises a template for amplification by a DNA polymerase. In some embodiments, the Cas nuclease or Cas nickase of the composition is guided to a target polynucleotide by the guide sequence and cleaves the target polynucleotide, and one strand of the cleaved target polynucleotide hybridizes to the primer binding sequence and serves as a primer for a DNA polymerase. In some embodiments, the DNA polymerase is capable of synthesizing a DNA strand complementary to the SOI to form a double-stranded sequence comprising the SOI. In some embodiments, the double-stranded sequence comprising the SOI is inserted into the cleaved target polynucleotide, e.g., via ligation or a DNA repair pathway described herein.

In some embodiments, the DNA template sequence is about 5 nucleotides to about 5000 nucleotides in length. In some embodiments, the DNA template sequence is about 6 nucleotides to about 1000 nucleotides in length. In some embodiments, the DNA template sequence is about 7 nucleotides to about 750 nucleotides in length. In some embodiments, the DNA template sequence is about 8 nucleotides to about 500 nucleotides in length. In some embodiments, the DNA template sequence is about 9 nucleotides to about 250 nucleotides in length. In some embodiments, the DNA template sequence is about 10 nucleotides to about 100 nucleotides in length. In some embodiments, the DNA template sequence is about 15 nucleotides to about 90 nucleotides in length. In some embodiments, the DNA template sequence is about 20 nucleotides to about 80 nucleotides in length. In some embodiments, the DNA template sequence is about 25 nucleotides to about 70 nucleotides in length. In some embodiments, the DNA template sequence is about 30 nucleotides to about 50 nucleotides in length. In some embodiments, the DNA template sequence is about 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the DNA template sequence is greater than about 10 nucleotides, greater than about 15 nucleotides, greater than about 20 nucleotides, greater than about 25 nucleotides, greater than about 30 nucleotides, greater than about 35 nucleotides, greater than about 40 nucleotides, greater than about 45 nucleotides, or greater than about 50 nucleotides in length.

In some embodiments, the DNA template sequence comprises a primer-binding sequence. In some embodiments, the primer-binding sequence is about 3 to about 50 nucleotides in length. In some embodiments, the primer-binding sequence is about 4 to about 45 nucleotides in length. In some embodiments, the primer-binding sequence is about 5 to about 40 nucleotides in length. In some embodiments, the primer-binding sequence is about 6 to about 35 nucleotides in length. In some embodiments, the primer-binding sequence is about 7 to about 30 nucleotides in length. In some embodiments, the primer-binding sequence is about 8 to about 25 nucleotides in length. In some embodiments, the primer-binding sequence is about 10 to about 20 nucleotides in length. In some embodiments, the primer-binding sequence is about 4 to about 30 nucleotides in length. In some embodiments, the primer-binding sequence is about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the primer-binding sequence is of sufficient length to hybridize with a region of the cleaved target DNA sequence.

In some embodiments, the polynucleotide comprising the DNA template sequence comprises a modified nucleotide, a non-B DNA structure, a DNA polymerase recruitment moiety, a DNA ligase recruitment moiety, or a combination thereof.

In some embodiments, the polynucleotide comprising DNA template sequence comprises a modified nucleotide. In some embodiments, the modified nucleotide comprises an abasic site, a covalent linker, a xeno nucleic acid (XNA), a locked nucleic acid (LNA), a peptide nucleic acid (PNA), a phosphorothioate bond, a DNA lesion, a DNA photoproduct, a modified deoxyribonucleoside, a methylated nucleotide, or a combination thereof.

In some embodiments, the modified nucleotide reduces or prevents overextension of the sequence of interest by the DNA polymerase. In some embodiments, reducing or preventing overextension of the sequence of interest by the DNA polymerase increases the precision of inserting the double-stranded sequence comprising the sequence of interest. In some embodiments, the modified nucleotide comprises an abasic site, also known as an apurinic/apyrimidinic (AP site).

In some embodiments, the modified nucleotide comprises a covalent linker. In some embodiments, the covalent linker comprises a triethylene glycol (TEG) linker. In some embodiments, the covalent linker comprises an amino linker. TEG linkers and amino linkers have been shown to block polymerase extension; see, e.g., Strobel et al., bioRxiv doi:10.1101/2019.12.26.888743 (23 Jan. 2020).

In some embodiments, the modified nucleotide reduces or prevents nuclease degradation of a polynucleotide of the disclosure. In some embodiments, the modified nucleotide comprises a xeno nucleic acid (XNA). An XNA is a synthetic nucleotide analogue that has a different sugar group than the deoxyribose of DNA or the ribose of RNA. Exemplary sugar groups for XNA include, but are not limited to, threose, cyclohexene, glycol, or a locked ribose. In some embodiments, the XNA comprises 1,5-anhydrohexitol nucleic acid (HNA), cyclohexene nucleic acid (CeNA), threose nucleic acid (TNA), glycol nucleic acid (GNA), locked nucleic acid (LNA), and peptide nucleic acid (PNA). In some embodiments, the modified nucleotide comprises a locked nucleic acid (LNA), also known as a bridged nucleic acid (BNA). An LNA is a modified RNA nucleotide in which the ribose moiety is modified with an extra bridge connecting the 2′ oxygen and 4′ carbon. In some embodiments, the modified nucleotide comprises a peptide nucleic acid (PNA). Unlike the deoxyribose or ribose backbones of DNA or RNA, the backbone of a PNA polymer comprises N-(2-aminoethyl)-glycine units linked by peptide bonds, and the purine and pyrimidine bases are linked to the PNA backbone by a methylene bridge and a carbonyl group. In some embodiments, the modified nucleotide comprises a phosphorothioate bond. A phosphorothioate bond comprises a sulfur atom in place of one of the oxygens in the phosphate group linking two nucleotides. In some embodiments, the presence of an XNA, e.g., an LNA or a PNA, or a phosphorothioate bond in a polynucleotide increases stability of the polynucleotide against nuclease degradation.

In some embodiments, the presence of a modified nucleotide in a polynucleotide (e.g., the polynucleotide of the composition provided herein) is capable of recruiting a DNA polymerase to the polynucleotide. In some embodiments, recruiting a DNA polymerase comprises: increasing the likelihood that a DNA polymerase recognizes the polynucleotide, e.g., due to presence of the modified nucleotide therein; promoting binding of a DNA polymerase to the polynucleotide; and/or activating a DNA polymerase, e.g., initiating or increasing activity of the DNA polymerase. In some embodiments, the recruited DNA polymerase binds to a strand of the cleaved target polynucleotide and extends the sequence of interest on the DNA template sequence, as described herein.

In some embodiments, the modified nucleotide comprises a DNA lesion. As used herein, a “DNA lesion” refers to a region of a DNA polynucleotide containing a base alteration, base deletion, and/or sugar alteration typically indicative of DNA damage. DNA lesions can be caused by hydrolysis, oxidation, alkylation, depurination, depyrimidination, and/or deamination of a nucleobase. In some embodiments, the DNA lesion is capable of recruiting a DNA polymerase. In some embodiments, the DNA lesion comprises 8-oxoguanine, thymine-glycol, N7-(2-hydroxethyl)guanine (7HEG), 7-(2-oxoethyl)guanine, or a combination thereof. In some embodiments, the DNA lesion comprises 8-oxoguanine, thymine-glycol, or a combination thereof.

In some embodiments, the modified nucleotide comprises a DNA photoproduct. DNA photoproducts are ultraviolet (UV)-induced DNA lesions and are further described in, e.g., Yokoyama et al., Int J Mol Sci 15(11):20321-20338 (2014). In some embodiments, the DNA photoproduct is capable of recruiting a DNA polymerase. In some embodiments, the DNA photoproduct comprises a pyrimidine dimer, a cyclobutane pyrimidine dimer (CPD), a pyrimidine (6-4) pyrimidone photoproduct (also referred to as a “(6-4) photoproduct”), an adenine-thymine heterodimer, a Dewar pyrimidinone, or a combination thereof. In some embodiments, the DNA photoproduct comprises CPD, a (6-4) photoproduct, or a combination thereof.

In some embodiments, the modified nucleotide comprises a modified deoxyribonucleoside. In some embodiments, the modified deoxyribonucleoside is capable of recruiting a DNA polymerase. In some embodiments, the modified deoxyribonucleoside comprises a base not typically present in DNA, i.e., adenine, cytosine, guanine, or thymine. In some embodiments, the modified deoxyribonucleoside comprises deoxyuridine, acrolein-deoxyguanine, malondialdehyde-deoxyguanine, deoxyinosine, deoxyxanthosine, or a combination thereof. In some embodiments, the modified deoxyribonucleoside comprises deoxyuridine.

In some embodiments, the modified nucleotide comprises one or more methylated nucleotides. In some embodiments, methylated nucleotides, e.g., methylated cytosines, are capable of recruiting a DNA polymerase. In some embodiments, the methylated nucleotide comprises 5-hydroxymethylcytosine, 5-methylcytosine, or a combination thereof.

In some embodiments, the DNA template sequence comprises a non-B DNA structure. As used herein, “a non-B DNA structure” is a DNA secondary structural conformation that is not the canonical right-handed B-DNA helix. Non-limiting examples of non-B DNA structures include G-quadruplex, triplex DNA (H-DNA), Z-DNA, cruciform, slipped DNA strands, A-tract bending, sticky DNA. Non-B DNA structures are further described in, e.g., Guiblet et al., Nucleic Acids Res 49(3):1497-1516 (2021). In some embodiments, the non-B DNA structure is capable of recruiting a DNA polymerase. In some embodiments, the non-B DNA structure comprises a hairpin, a cruciform, Z-DNA, H-DNA (triplex DNA), G-quadruplex DNA (tetraplex DNA), slipped DNA, sticky DNA, or a combination thereof.

In some embodiments, the DNA template sequence comprises a DNA polymerase recruitment moiety. DNA polymerase recruitment is described herein. Non-limiting examples of DNA polymerases that can be recruited by the DNA polymerase recruitment moiety include bacterial DNA polymerases such as Pol I (including a Klenow fragment thereof), Pol II, Pol III, Pol IV, or Pol V; eukaryotic DNA polymerases such as Pol α, Pol β, Pol λ, Pol γ, Pol σ, Pol μ, Pol δ, Pol ε, Pol η, Pol τ, Pol κ, Po ζ, Pol θ, REV1, or REV3; isothermal DNA polymerases such as Bst, T4, or Φ29 (phi29) DNA polymerase; thermostable DNA polymerases such as Taq, Pfu, KOD, Tth, or Pwo DNA polymerase; or a variant or homologue thereof.

In some embodiments, a polynucleotide of the disclosure can be chemically crosslinked to one or more moieties or conjugates which enhance the activity, cellular distribution, or cellular uptake of the polynucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

A conjugate may include a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the amino terminus of an exogenous polypeptide (e.g., a site-directed modifying polypeptide). In some embodiments, a PTD is covalently linked to the carboxyl terminus of an exogenous polypeptide (e.g., a site-directed modifying polypeptide). In some embodiments, a PTD is covalently linked to a nucleic acid (e.g., a DNA-targeting RNA, a polynucleotide encoding a DNA-targeting RNA, a polynucleotide encoding a site-directed modifying polypeptide, etc.). Exemplary PTDs include but are not limited to a minimal undecapeptide protein transduction domain (corresponding to residues 47-57 of HIV-1 TAT comprising YGRKKRRQRRR; SEQ ID NO:7); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); an Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:8); Transportan GWTLNSAGYLLGKINLKALAALAKKIL (SEQ ID NO:9); KALAWEAKLAKALAKALAKHLAKALAKALKCEA (SEQ ID NO: 10); and RQIKIWFQNRRMKWKK (SEQ ID NO: 11). Exemplary PTDs include but are not limited to, YGRKKRRQRRR (SEQ ID NO: 12), RKKRRQRRR (SEQ ID NO:13); an arginine homopolymer of from 3 arginine residues to 50 arginine residues; Exemplary PTD domain amino acid sequences include, but are not limited to, any of the following: YGRKKRRQRRR (SEQ ID NO:14); RKKRRQRR (SEQ ID NO: 15); YARAAARQARA (SEQ ID NO: 16); THRLPRRRRRR (SEQ ID NO:17); and GGRRARRRRRR (SEQ ID NO: 18). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

In some embodiments, a polynucleotide of the disclosure is codon optimized for expression in a eukaryotic cell. In some embodiments, the polynucleotide sequence encoding a stiCas9 is codon optimized for expression in an animal cell. In some embodiments, the polynucleotide sequence encoding the recombinant Cas effector protein is codon optimized for expression in a human cell. In some embodiments, the polynucleotide sequence encoding the recombinant Cas effector protein is codon optimized for expression in a plant cell. Codon optimization is the adjustment of codons to match the expression host's tRNA abundance in order to increase yield and efficiency of recombinant or heterologous protein expression. Codon optimization methods are routine in the art and may be performed using software programs such as, for example, Integrated DNA Technologies' Codon Optimization tool, Entelechon's Codon Usage Table analysis tool, GENEMVAKER's Blue Heron software, Aptagen's Gene Forge software, DNA Builder Software, General Codon Usage Analysis software, the publicly available OPTIMIZER software, and Genscript's OptimumGene algorithm.

### CRISPR-Cas Systems

In some embodiments, the present disclosure encompasses CRISPR-Cas systems comprising a naturally-occurring Cas effector protein or a non-naturally occurring Cas effector protein, and a polynucleotide encoding a sequence of interest. In some embodiments, the CRISPR-Cas system comprises a naturally-occurring Cas effector protein or non-naturally occurring Cas effector protein, a polynucleotide encoding a sequence of interest, and a polynucleotide capable of forming a complex with a Cas effector protein. In some embodiments, the polynucleotide capable of forming a complex with a Cas effector protein comprises a guide sequence, a Cas-binding region, and a DNA template region.

In some embodiments, the CRISPR-Cas system comprises a regulatory element operably linked to a polynucleotide sequence encoding a recombinant Cas effector protein provided herein, and polynucleotide that forms a complex with the recombinant Cas effector protein and includes a guide sequence.

In some embodiments, the regulatory element linked to the polynucleotide sequence encoding a recombinant Cas effector protein is a promoter. In some embodiments, the regulatory element is a eukaryote promoter. In some embodiments, the regulatory element is a viral promoter. In some embodiments, the regulatory element is a eukaryotic regulatory element, i.e., a eukaryotic promoter. In some embodiments, the eukaryotic regulatory element is a mammalian promoter.

In some embodiments, the polynucleotide capable of forming a complex with the Cas effector protein of the CRISPR-Cas system is an RNA molecule. An RNA molecule that binds to CRISPR-Cas components and targets them to a specific location within the target DNA is referred to herein as “guide RNA,” “gRNA,” or “small guide RNA” and may also be referred to herein as a “DNA-targeting RNA.” A guide polynucleotide, e.g., guide RNA, includes at least two nucleotide segments: at least one “DNA-binding segment” and at least one “polypeptide-binding segment.” By “segment” is meant a part, section, or region of a molecule, e.g., a contiguous stretch of nucleotides of guide polynucleotide molecule. The definition of “segment,” unless otherwise specifically defined, is not limited to a specific number of total base pairs.

In some embodiments, the DNA-binding segment (or “DNA-targeting sequence”) of the guide polynucleotide hybridizes with a target sequence in a cell. In some embodiments, the DNA-binding segment of the guide polynucleotide, e.g., guide RNA, includes a polynucleotide sequence that is complementary to a specific sequence within a target DNA.

In some embodiments, the guide polynucleotide of the present disclosure has a guide sequence that hybridizes to a target sequence in a eukaryotic cell. In some embodiments, the eukaryotic cell is an animal or human cell. In some embodiments, the eukaryotic cell is a human or rodent or bovine cell line or cell strain. Examples of such cells, cell lines, or cell strains include, but are not limited to, mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C₁₂₇, L cell, COS, e.g., COS1 and COS7, QC1-3, HEK-293, VERO, PER.C₆, HeLA, EB1, EB2, EB3, oncolytic or hybridoma-cell lines. In some embodiments, the eukaryotic cells are CHO-cell lines. In some embodiments, the eukaryotic cell is a CHO cell. In some embodiments, the cell is a CHO-K1 cell, a CHO-K1 SV cell, a DG44 CHO cell, a DUXB1 1 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the POTELLIGENT CHOK1 SV (Lonza Biologics, Inc.). Eukaryotic cells can also be avian cells, cell lines or cell strains, such as, for example, EBX cells, EB14, EB24, EB26, EB66, or EBv13.

In some embodiments, the eukaryotic cell is a human cell. In some embodiments, the human cell is a stem cell. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs). In some embodiments, the human cell is a differentiated form of any of the cells described herein. In some embodiments, the eukaryotic cell is a cell derived from any primary cell in culture.

In some embodiments, the eukaryotic cell is a hepatocyte such as a human hepatocyte, animal hepatocyte, or a non-parenchymal cell. For example, the eukaryotic cell can be a plateable metabolism qualified human hepatocyte, a plateable induction qualified human hepatocyte, plateable human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C₅₇B1I/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes).

In some embodiments, the eukaryotic cell is a plant cell. For example, the plant cell can be of a crop plant such as cassava, corn, sorghum, wheat, or rice. The plant cell can be of an algae, tree, or vegetable. The plant cell can be of a monocot or dicot or of a crop or grain plant, a production plant, fruit, or vegetable. For example, the plant cell can be of a tree, e.g., a citrus tree such as orange, grapefruit, or lemon tree; peach or nectarine trees; apple or pear trees; nut trees such as almond or walnut or pistachio trees; nightshade plants, e.g., potatoes, plants of the genus Brassica, plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

In some embodiments, the guide sequence of the guide polynucleotide is about 5 to about 50 nucleotides. In some embodiments, the guide sequence of the guide polynucleotide is about 6 to about 45 nucleotides. In some embodiments, the guide sequence of the guide polynucleotide is about 7 to about 40 nucleotides. In some embodiments, the guide sequence of the guide polynucleotide is about 8 to about 35 nucleotides. In some embodiments, the guide sequence of the guide polynucleotide is about 9 to about 30 nucleotides. In some embodiments, the guide sequence of the guide polynucleotide is about 10 to about 20 nucleotides. In some embodiments, the guide sequence of the guide polynucleotide is about 12 to about 20 nucleotides. In some embodiments, the guide sequence of the guide polynucleotide is about 14 to about 20 nucleotides. In some embodiments, the guide sequence of the guide polynucleotide is about 16 to about 20 nucleotides. In some embodiments, the guide sequence of the guide polynucleotide is about 18 to about 20 nucleotides. In some embodiments, the guide sequence of the guide polynucleotide is about 5 to about 10 nucleotides. In some embodiments, the guide sequence of the guide polynucleotide is about 6 to about 10 nucleotides. In some embodiments, the guide sequence of the guide polynucleotide is about 7 to about 10 nucleotides. In some embodiments, the guide sequence of the guide polynucleotide is about 8 to about 10 nucleotides. The length of the guide sequence may be determined by the skilled artisan using guide sequence design tools such as, e.g., CRISPR Design Tool (Hsu et al., Nat Biotechnol 31(9):827-832 (2013)), ampliCan (Labun et al., bioRxiv 2018, doi: 10.1101/249474), CasFinder (Alach et al., bioRxiv 2014, doi: 10.1101/005074), CHOPCHOP (Labun et al., Nucleic Acids Res 2016, doi: 10.1093/nar/gkw398), and the like.

In some embodiments, the guide polynucleotide, e.g., guide RNA, of the present disclosure includes a polypeptide-binding sequence/segment. The polypeptide-binding segment (or “protein-binding sequence”) of the guide polynucleotide, e.g., guide RNA, interacts with the polynucleotide-binding domain of a Cas effector protein of the present disclosure. Such polypeptide-binding segments or sequences are known to those of skill in the art, e.g., those disclosed in U.S. Patent Publications 2014/0068797, 2014/0273037, 2014/0273226, 2014/0295556, 2014/0295557, 2014/0349405, 2015/0045546, 2015/0071898, 2015/0071899, and 2015/0071906, the disclosures of which are incorporated herein in their entireties. In some embodiments, the polypeptide-binding segment of the guide polynucleotide binds to Cas9. In some embodiments, the polypeptide-binding segment of the guide polynucleotide binds to the recombinant Cas9 proteins provided herein.

In some embodiments, the guide polynucleotide is at least about 10, 15, 20, 25 or 30 nucleotides and up to about 25, 30, 35, 40, 45, 50, 60, 70, 80, 90, 100, 110, 120, 130, 140 or 150 nucleotides. In some embodiments, the guide polynucleotide is between about 10 to about 150 nucleotides. In some embodiments, the guide polynucleotide is between about 20 to about 120 nucleotides. In some embodiments, the guide polynucleotide is between about 30 to about 100 nucleotides. In some embodiments, the guide polynucleotide is between about 40 to about 80 nucleotides. In some embodiments, the guide polynucleotide is between about 50 to about 60 nucleotides. In some embodiments, the guide polynucleotide is between about 10 to about 35 nucleotides. In some embodiments, the guide polynucleotide is between about 15 to about 30 nucleotides. In some embodiments, the guide polynucleotide is between about 20 to about 25 nucleotides.

The guide polynucleotide, e.g., guide RNA, can be introduced into the target cell as an isolated molecule, e.g., RNA molecule, or is introduced into the cell using an expression vector containing DNA encoding the guide polynucleotide, e.g., guide RNA.

In some embodiments, the guide polynucleotide of the CRISPR-Cas system is linked to a direct repeat sequence. A direct repeat, or DR, sequence is an array of repetitive sequences in the CRISPR locus, interspaced by short stretches of non-repetitive sequences (spacers). The spacer sequences target the Protospacer Adjacent Motifs (PAM) on the target sequence. When the non-coding portion of the CRISPR locus (i.e., the guide polynucleotide and the tracrRNA) is transcribed, the transcript is cleaved at the DR sequences into short crRNAs containing individual spacer sequences, which direct the Cas9 nuclease to the PAM. In some embodiments, the DR sequence is RNA. In some embodiments, the DR sequence is encoded by a nucleic acid. In some embodiments, the DR sequence is linked to the guide polynucleotide. In some embodiments, the DR sequence is linked to the guide sequence of the guide polynucleotide. In some embodiments, the DR sequence includes a secondary structure. In some embodiments, the DR sequence includes a stem loop structure. In some embodiments, the DR sequence is 10 to 20 nucleotides. In some embodiments, the DR sequence is at least 16 nucleotides. In some embodiments, the DR sequence is at least 16 nucleotides and includes a single stem loop. In some embodiments, the DR sequence includes an RNA aptamer. In some embodiments, the secondary structure or stem loop in the DR is the recognized by a nuclease for cleavage. In some embodiments, the nuclease is a ribonuclease. In some embodiments, the nuclease is RNase III.

In some embodiments, the CRISPR-Cas systems of the present disclosure further include a tracrRNA. A “tracrRNA,” or trans-activating CRISPR-RNA, forms an RNA duplex with a pre-crRNA, or pre-CRISPR-RNA, and is then cleaved by the RNA-specific ribonuclease RNase III to form a crRNA/tracrRNA hybrid. In some embodiments, the guide RNA includes the crRNA/tracrRNA hybrid. In some embodiments, the tracrRNA component of the guide RNA activates the Cas effector protein. In some embodiments, the guide polynucleotide of the CRISPR-Cas system includes a tracrRNA sequence. In some embodiments, the CRISPR-Cas system includes a separate polynucleotide including a tracrRNA sequence.

In some embodiments, the polynucleotide encoding a recombinant Cas effector protein and a guide polynucleotide is on a single vector. In some embodiments, the polynucleotide encoding a recombinant Cas effector protein, a guide polynucleotide (or nucleotide that can be transcribed into a guide polynucleotide), and a tracrRNA are on a single vector. In some embodiments, the polynucleotide encoding a recombinant Cas effector protein, a guide polynucleotide (or nucleotide that can be transcribed into a guide polynucleotide), a tracrRNA, and a direct repeat sequence are on a single vector. In some embodiments, the vector is an expression vector. In some embodiments, the vector is a mammalian expression vector. In some embodiments, the vector is a human expression vector. In some embodiments, the vector is a plant expression vector.

In some embodiments, the polynucleotide encoding a recombinant Cas effector protein and a guide polynucleotide is a single nucleic acid molecule. In some embodiments, the polynucleotide encoding a recombinant Cas effector protein, a guide polynucleotide, and a tracrRNA is a single nucleic acid molecule. In some embodiments, the polynucleotide encoding a recombinant Cas effector protein, a guide polynucleotide, a tracrRNA, and a direct repeat sequence is a single nucleic acid molecule. In some embodiments, the single nucleic acid molecule is an expression vector. In some embodiments, the single nucleic acid molecule is a mammalian expression vector. In some embodiments, the single nucleic acid molecule is a human expression vector. In some embodiments, the single nucleic acid molecule is a plant expression vector.

In some embodiments, the recombinant Cas effector protein and the guide polynucleotide are capable of forming a complex. In some embodiments, the complex of the recombinant Cas effector protein and the guide polynucleotide does not occur in nature.

### Cells

In some embodiments of the disclosure, the eukaryotic cell is a eukaryotic cell. In some embodiments, the eukaryotic cell is an animal or human cell. In some embodiments, the eukaryotic cell is a human or rodent or bovine cell line or cell strain. Examples of such cells, cell lines, or cell strains include, but are not limited to, mouse myeloma (NSO)-cell lines, Chinese hamster ovary (CHO)-cell lines, HT1080, H9, HepG2, MCF7, MDBK Jurkat, NIH3T3, PC12, BHK (baby hamster kidney cell), VERO, SP2/0, YB2/0, Y0, C₁₂₇, L cell, COS, e.g., COS1 and COS7, QC1-3, HEK-293, VERO, PER.C₆, HeLa, EB1, EB2, EB3, oncolytic or hybridoma-cell lines. In some embodiments, the eukaryotic cells are CHO-cell lines. In some embodiments, the eukaryotic cell is a CHO cell. In some embodiments, the cell is a CHO-K1 cell, a CHO-K1 SV cell, a DG44 CHO cell, a DUXB11 CHO cell, a CHOS, a CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the POTELLIGENT CHOK1 SV (Lonza Biologics, Inc.). Eukaryotic cells can also be avian cells, cell lines or cell strains, such as, for example, EBX cells, EB14, EB24, EB26, EB66, or EBv13.

In some embodiments, the eukaryotic cell is a human cell. In some embodiments, the human cell is a stem cell. The stem cells can be, for example, pluripotent stem cells, including embryonic stem cells (ESCs), adult stem cells, induced pluripotent stem cells (iPSCs), tissue specific stem cells (e.g., hematopoietic stem cells) and mesenchymal stem cells (MSCs). In some embodiments, the cell is a pluripotent stem cell. In some embodiments, the cell is an induced pluripotent stem cell. In some embodiments, the human cell is a differentiated form of any of the cells described herein. In some embodiments, the eukaryotic cell is a cell derived from any primary cell in culture.

In some embodiments, the eukaryotic cell is a hepatocyte such as a human hepatocyte, animal hepatocyte, or a non-parenchymal cell. For example, the eukaryotic cell can be a plateable metabolism qualified human hepatocyte, a plateable induction qualified human hepatocyte, plateable human hepatocyte, suspension qualified human hepatocyte (including 10-donor and 20-donor pooled hepatocytes), human hepatic kupffer cells, human hepatic stellate cells, dog hepatocytes (including single and pooled Beagle hepatocytes), mouse hepatocytes (including CD-1 and C₅₇B1I/6 hepatocytes), rat hepatocytes (including Sprague-Dawley, Wistar Han, and Wistar hepatocytes), monkey hepatocytes (including Cynomolgus or Rhesus monkey hepatocytes), cat hepatocytes (including Domestic Shorthair hepatocytes), and rabbit hepatocytes (including New Zealand White hepatocytes).

In some embodiments, the eukaryotic cell is a hematopoietic cell. In some embodiments, the hematopoietic cell is a myeloid progenitor cell. In some embodiments, the hematopoietic cell is a lymphoid progenitor cell. In some embodiments, the hematopoietic cell is a mast cell, a megakarytocyte, a thrombocyte, basophil, a neutrophil, an eosinophil, a dendritic cell, a monocyte, or a macrophage. In some embodiments, the hematopoietic cell is a natural killer cell (NK cell), a T lymphocyte, or a B lymphocyte. In some embodiments, the T or B lymphocyte comprises a chimeric antigen receptor (CAR).

In some embodiments, the eukaryotic cell is a plant cell. For example, the plant cell can be of a crop plant such as cassava, corn, sorghum, wheat, or rice. The plant cell can be of an algae, tree, or vegetable. The plant cell can be of a monocot or dicot or of a crop or grain plant, a production plant, fruit, or vegetable. For example, the plant cell can be of a tree, e.g., a citrus tree such as orange, grapefruit, or lemon tree; peach or nectarine trees; apple or pear trees; nut trees such as almond or walnut or pistachio trees; nightshade plants, e.g., potatoes, plants of the genus Brassica, plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

In some embodiments, the eukaryotic cell is a tissue culture of any of the aforementioned cells. In some embodiments, the eukaryotic cell is in the form of a tissue extract of any of the aforementioned cells.

In some embodiments, the eukaryotic cell comprises a genomically-integrated Cas polynucleotide. In some embodiments, the eukaryotic cell comprises an inducible genomically-integrated Cas polynucleotide.

### Delivery Systems

Various methods are known in the art for delivery of CRISPR-Cas systems. Suitable delivery systems include microinjection, electroporation, transfection, or hydrodynamic delivery of a polynucleotide encoding a Cas effector protein, a polynucleotide comprising a sequence of interest, and/or a polynucleotide capable of forming a complex with a Cas effector protein. In some embodiments, the delivery system comprises a delivery particle. Examples of such delivery systems, including nanoparticles, cell-penetrating peptides, and DNA nanoclews, are disclosed in Lino et al., Drug Delivery, 25(1):1234-1257 (2018)).

In some embodiments, the CRISPR-Cas system, including a Cas effector protein, a polynucleotide encoding a Cas effector protein, a polynucleotide encoding a sequence of interest, and/or a polynucleotide capable of forming a complex with a Cas effector protein, of the present disclosure is delivered by a delivery particle. A delivery particle is a biological delivery system or formulation which includes a particle. A “particle,” as defined herein, is an entity having a maximum diameter of about 100 microns (μm). In some embodiments, the particle has a maximum diameter of about 10 μm. In some embodiments, the particle has a maximum diameter of about 2000 nanometers (nm). In some embodiments, the particle has a maximum diameter of about 1000 nm. In some embodiments, the particle has a maximum diameter of about 900 nm, about 800 nm, about 700 nm, about 600 nm, about 500 nm, about 400 nm, about 300 nm, about 200 nm, or about 100 nm. In some embodiments, the particle has a diameter of about 25 nm to about 200 nm. In some embodiments, the particle has a diameter of about 50 nm to about 150 nm. In some embodiments, the particle has a diameter of about 75 nm to about 100 nm.

Delivery particles may be provided in any form, including but not limited to: solid, semi-solid, emulsion, or colloidal particles. In some embodiments, the delivery particle is a lipid-based system, a liposome, a micelle, a microvesicle, an exosome, or a gene gun. In some embodiments, the delivery particle includes a CRISPR-Cas system. In some embodiments, the delivery particle includes a CRISPR-Cas system including a recombinant Cas effector protein and a polynucleotide capable of forming a complex with the Cas effector protein, wherein said polynucleotide comprises a guide polynucleotide. In some embodiments, the delivery particle includes a Cas effector protein, a polynucleotide comprising a sequence of interest, and a polynucleotide capable of forming a complex with a Cas effector protein and comprising a guide polynucleotide. In some embodiments, the delivery particle includes a CRISPR-Cas system including a recombinant Cas effector protein and a polynucleotide which forms a complex with a Cas effector protein and which comprises a guide polynucleotide, wherein the recombinant Cas effector protein and the polynucleotide are in a complex. In some embodiments, the delivery particle includes a CRISPR-Cas system including a recombinant Cas effector protein, a polynucleotide which forms a complex with a Cas effector protein and which comprises a guide polynucleotide, and polynucleotide including a tracrRNA. In some embodiments, the delivery particle includes a CRISPR-Cas system including a Cas effector protein, a polynucleotide which forms a complex with a Cas effector protein and comprises a guide polynucleotide, and a tracrRNA.

In some embodiments, the complex of the Cas effector protein and a polynucleotide of the disclosure is a ribonucleoprotein (RNP), wherein said RNP is delivered via hydrodynamic delivery, a nanoparticle, a vesicle, a cell-penetrating peptide, or a DNA nanoclew.

In some embodiments, the delivery particle further includes a lipid, a sugar, a metal or a protein. In some embodiments, the delivery particle is a lipid envelope. Delivery of mRNA using lipid envelopes or delivery particles including lipids is described, for example, in Su et al., Molecular Pharmacology 8(3):774-784 (2011). In some embodiments, the delivery particle is a sugar-based particle, for example, GalNAc. Sugar-based particles are described in WO 2014/118272 and Nair et al., J. Am. Chem. Soc. 136(49):16958-16961 (2014).

In some embodiments, the delivery particle is a nanoparticle. Nanoparticles encompassed in the present disclosure may be provided in different forms, e.g., as solid nanoparticles (e.g., metal such as silver, gold, iron, titanium), non-metal, lipid-based solids, polymers, suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present disclosure.

Preparation of delivery particles is further described in U.S. Patent Publications 2011/0293703, 2012/0251560, and 2013/0302401; and U.S. Pat. Nos. 5,543,158, 5,855,913, 5,895,309, 6,007,845, and 8,709,843.

In some embodiments, a vesicle includes the CRISPR-Cas system of the present disclosure. A “vesicle” is a small structure within a cell having a fluid enclosed by a lipid bilayer. In some embodiments, the CRISPR-Cas system of the present disclosure is delivered by a vesicle. In some embodiments, the vesicle includes a recombinant Cas effector protein and a guide polynucleotide. In some embodiments, the vesicle includes a Cas effector protein and a guide polynucleotide, wherein the Cas effector protein and the guide polynucleotide are in a complex. In some embodiments, the vesicle includes a CRISPR-Cas system including a Cas effector protein, a polynucleotide capable of forming a complex with a Cas effector protein and comprising a guide polynucleotide, and a polynucleotide including a tracrRNA. In some embodiments, the vesicle includes a CRISPR-Cas system including a t Cas effector protein, a polynucleotide capable of forming a complex with a Cas effector protein and comprising guide polynucleotide, and a tracrRNA.

In some embodiments, the vesicle including the Cas effector protein and polynucleotide capable of forming a complex with the Cas effector protein and comprising a guide polynucleotide is an exosome or a liposome. In some embodiments, the vesicle is an exosome. In some embodiments, the exosome is used to deliver the CRISPR-Cas systems of the present disclosure. Exosomes are endogenous nano-vesicles (i.e., having a diameter of about 30 to about 100 nm) that transport RNAs and proteins, and which can deliver RNA to the brain and other target organs. Engineered exosomes for delivery of exogenous biological materials into target organs is described, for example, by Alvarez-Erviti et al., Nature Biotechnology 29:341 (2011), E1-Andaloussi et al., Nature Protocols 7:2112-2116 (2012), and Wahlgren et al., Nucleic Acids Research 40(17):e130 (2012).

In some embodiments, the liposome is used to deliver the CRISPR-Cas systems of the present disclosure. Liposomes are spherical vesicle structures having at least one lipid bilayer and can be used as a vehicle for administration of nutrients and pharmaceutical drugs. Liposomes are often composed of phospholipids, in particular phosphatidylcholine, but also other lipids such as egg phosphatidylethanolamine. Types of liposomes include, but are not limited to, multilamellar vesicle, small unilamellar vesicle, large unilamellar vesicle, and cochleate vesicle. See, e.g., Spuch and Navarro, Journal of Drug Delivery, Article ID 469679 (2011). Liposomes for delivery of biological materials such as CRISPR-Cas components are described, for example, by Morrissey et al., Nature Biotechnology 23(8):1002-1007 (2005), Zimmerman et al., Nature Letters 441:111-114 (2006), and Li et al., Gene Therapy 19:775-780 (2012).

In some embodiments, the Cas effector protein can be delivered using cell-penetrating peptide fused to the Cas effector protein.

In some embodiments, the Cas effector protein and a polynucleotide of the disclosure can be delivered in the form of a DNA nanoclew. DNA nanoclews are spherical structures comprising DNA that can be loaded with a payload, such as a Cas effector protein (Sun et al., J. Am. Chem. Soc., 136:14722-14725). DNA nanoclews have been used in vitro for delivery of Cas9 editing systems (Lino et al., Drug Delivery, 25(1):1234-1257).

In some embodiments, a viral vector includes the CRISPR-Cas systems of the present disclosure. In some embodiments, the CRISPR-Cas system of the present disclosure is delivered by a viral vector. In some embodiments, the viral vector includes a recombinant Cas9 and a guide polynucleotide. In some embodiments, the viral vector includes a Cas effector protein and a guide polynucleotide, wherein the Cas effector protein and the guide polynucleotide are in a complex. In some embodiments, the viral vector includes a CRISPR-Cas system including a Cas effector protein, a polynucleotide capable of forming a complex with a Cas effector protein and comprising a guide polynucleotide, and a polynucleotide including a tracrRNA. In some embodiments, the viral vector includes a CRISPR-Cas system including a Cas effector protein, a polynucleotide capable of forming a complex with a Cas effector protein and comprising a guide polynucleotide, and a tracrRNA. In some embodiments, the viral vector is of a retrovirus, a lentivirus, an adenovirus, or an adeno-associated virus. Examples of viral vectors are provided herein.

In some embodiments, retroviral, lentiviral, adenoviral, and/or adeno-associated virus (AAV) vectors can be used as a viral vector including the elements of the CRISPR-Cas systems as described herein. In some embodiments of the present disclosure, the Cas effector protein is expressed intracellularly by cells transduced by a viral vector.

In some embodiments, the Cas proteins and methods of the present disclosure are used in ex vivo gene editing, such as CAR-T type therapies. These embodiments may involve modification of cells from human donors. In these instances, viral vectors can be also used; however, there is the additional option to directly transfect the Cas9 protein (along with in vitro transcribed guide RNA and donor DNA) into cultured cells.

### Inhibitors of the Micro-Homology End Joining (MMEJ) Pathway

As used herein, an inhibitor of the MMEJ pathway is any compound, molecule, or entity that inhibits, antagonizes, blocks, or decreases the activity and/or level of any component of the MMEJ pathway. In some embodiments, the inhibitor of the MMEJ pathway is a PolQ inhibitor.

In some embodiments, the inhibitor of the MMEJ pathway is an inhibitor of POL Q/DNA polymerase q.

In some embodiments, the inhibitor of POL Q is a compound of formula (I):

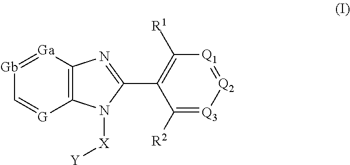

or any stereoisomer thereof or pharmaceutically acceptable salt thereof;
wherein,
R¹ and R² are each, independently, H, halo, C₁-C₃ alkyl, C₁-C₃ alkoxy, C₁-C₃ haloalkyl, C₁-C₃ hydroxyalkyl, —CN, C₂-C₄ alkyne, or C₂-C₆ alkoxyalkyl;
Q¹, Q², and Q³ are, independently N, C-L-R, or CR^{x}, wherein no more than one of Q¹, Q², and Q³ is C-L-R;
L is a bond, —O—; —C(O)—; —O(CH₂)ₚC(O)—; —C(O)NR^{y}—; —O(CH₂)ₚC(O)NR^{y}—; —O(CH₂)ₚNR^{y}; —NR^{y}—; —(CH₂)ₚ—; —(CH₂)ₚNR^{y}—; —(CH₂)ₚO—; —(CH₂)ₚC(O)—; —(CH₂)ₚC(O)O—; —O(CH₂)ₚ—;
p is, independently, 1, 2, or 3
R is H, R^{a}, R^{b}, R^{c}, or R^{d};
R^{a} is a 3-10 membered heterocycle optionally substituted with 1 to 4 substituents selected from amino, carboxy, halo, hydroxy, oxo, —CN, —S(O)₂OH, C₁-C₄ alkylamino, C₁-C₅ alkoxy, C₂-C₅ alkoxyalkyl, 4-6 membered heterocycle, and C₁-C₇ alkyl, wherein the C₁-C₇ alkyl is optionally substituted with 1 to 4 substituents selected from amino, carboxy, halo, hydroxy, oxo, —CN, C₂-C₈ ester, and C₁-C₅ alkoxy;
   R^{b} is a C₁-C₇ alkyl, wherein one or two methylene groups from the C₁-C₇ alkyl are optionally replaced with NR^{e} or 0 and one or two single bonds in a C₂-C₇ alkyl chain are optionally replaced with a double or triple bond(s), wherein the C₁-C₇ alkyl is optionally substituted with 1 to 4 substituents selected from: halo, oxo, hydroxy, carboxyl, amino, —CN, C₂-C₄ alkynyl, C₂-C₆ carbamate, C₁-C₈ amide, C₁-C₄ sulfonyl, C₁-C₄ sulfonamide, C₁-C₄ alkylamino, C₁-C₅ alkoxy, C₃-C₆ carbocycle, and 3-10 membered heterocycle,
wherein the C₃-C₆ carbocycle is optionally substituted with 1 to 4 substituents selected from hydroxy, halo, and carboxy;
wherein the 3-10 membered heterocycle is optionally substituted with 1 to 4 substituents selected from amino, carboxy, halo, hydroxy, oxo, —CN, —S(O)₂OH, C₁-C₄ alkylamino, C₁-C₅ alkoxy, C₂-C₅ alkoxyalkyl, 4-6 membered heterocycle, and C₁-C₇ alkyl, wherein the C₁-C₇ alkyl is optionally substituted with 1 to 4 substituents selected from amino, carboxy, halo, hydroxy, oxo, —CN, C₂-C₈ ester, and C₁-C₅ alkoxy;
R^{c} is a C₃-C₆ carbocycle optionally substituted with 1 to 4 substituents selected from hydroxy halo, and carboxy;
R^{d} is C₁-C₄ sulfonyl or C₁-C₄ sulfonamide;
R^{y} is H, C₁-C₃ alkyl, or C₁₋₃ haloalkyl;
R^{x} is H, halo, hydroxy, —CN, —NH₂, C₁-C₃ alkoxy, C₁-C₃ alkyl, or C₁₋₃ haloalkyl;
R^{e} is H, halo, C₁-C₈ alkyl, or C₁-C₈ haloalkyl;
X is a C₁-C₄ alkylene;
Y is a phenyl or 5-6 membered heteroaryl, wherein the phenyl or heteroaryl is optionally substituted with 1-3 substituents selected from halo, C₁-C₃ alkyl, C₁-C₃ alkoxy, —CN, C₁-C₃ haloalkyl, and cyclopropyl;
G is N or CH;
Gₐ and G_{b} are N, CH, or CR⁵ wherein one, and only one, of Gₐ and G_{b} is N or CH and one, and only one, of Gₐ and G_{b} is CR⁵;
R⁵ is

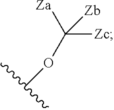

Zₐ and Z_{b} are, independently, C₁-C₃ alkyl or C₁-C₃ haloalkyl, or Zₐ and Z_{b} form a 3-6 membered carbocycle or heterocycle; and
Z_{c} is H, —CN, C₁-C₃ alkyl, C₁-C₃ haloalkyl, or C₂-C₄ alkyne,
or combinations thereof. In some embodiments the inhibitor of POL Q is a compound disclosed herein, or combinations thereof.

In some embodiments, the inhibitor of POL Q is a compound listed in Table I, a pharmaceutically acceptable salt thereof, or combinations thereof.

In some embodiments, the inhibitor of POL Q is a compound listed in Table I, Table II, Table III, a pharmaceutically acceptable salt thereof, or combinations thereof.

In some embodiments the inhibitor of POL Q is a compound of formula (I), or any stereoisomer thereof or pharmaceutically acceptable salt thereof, wherein G is N.

In some embodiments the inhibitor of POL Q is a compound of formula (I), or any stereoisomer thereof or pharmaceutically acceptable salt thereof, wherein G is CH.

In some embodiments the inhibitor of POL Q is a compound of formula (I), or any stereoisomer thereof or pharmaceutically acceptable salt thereof, wherein Gₐ is CR₅.

In some embodiments the inhibitor of POL Q is a compound of formula (I), or any stereoisomer thereof or pharmaceutically acceptable salt thereof, wherein G_{b} is CR₅.

In some embodiments the inhibitor of POL Q is a compound of formula (I), or any stereoisomer thereof or pharmaceutically acceptable salt thereof, wherein G_{b} is N.

In some embodiments the inhibitor of POL Q is a compound of formula (I), or any stereoisomer thereof or pharmaceutically acceptable salt thereof, wherein G_{b} is CH.

In some embodiments the inhibitor of POL Q is a compound of formula (I), or any stereoisomer thereof or pharmaceutically acceptable salt thereof, wherein G_{b} is N.

In some embodiments the inhibitor of POL Q is a compound of formula (I), or any stereoisomer thereof or pharmaceutically acceptable salt thereof, wherein G_{b} is CH.

In some embodiments the inhibitor of POL Q is a compound of formula (I), or any stereoisomer thereof or pharmaceutically acceptable salt thereof, wherein Zₐ and Z_{b}, are independently, C₁-C₃ alkyl.

In some embodiments the inhibitor of POL Q is a compound of formula (I), or any stereoisomer thereof or pharmaceutically acceptable salt thereof, wherein Zₐ and Z_{b} are —CH₃.

In some embodiments the inhibitor of POL Q is a compound of formula (I), or any stereoisomer thereof or pharmaceutically acceptable salt thereof, wherein Gₐ or G_{b} is CR₅ and R⁵ is

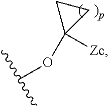

wherein p is 1-4.

In some embodiments the inhibitor of POL Q is a compound of formula (I), or any stereoisomer thereof or pharmaceutically acceptable salt thereof, wherein Gₐ or G_{b} is CR₅ and R⁵ is

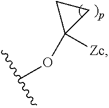

wherein p is 2.

In some embodiments the inhibitor of POL Q is a compound of formula (I), or any stereoisomer thereof or pharmaceutically acceptable salt thereof, wherein Gₐ or G_{b} is CR₅ and R⁵ is

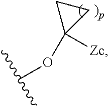

wherein p is is 1.

In some embodiments the inhibitor of POL Q is a compound of formula (I), or any stereoisomer thereof or pharmaceutically acceptable salt thereof, wherein Z_{c} is —CH₃.

In some embodiments the inhibitor of POL Q is a compound of formula (I), or any stereoisomer thereof or pharmaceutically acceptable salt thereof, wherein Z_{c} is —CN.

In some embodiments the inhibitor of POL Q is a compound of formula (I), or any stereoisomer thereof or pharmaceutically acceptable salt thereof, wherein Y is a phenyl or 5-6 membered heteroaryl, wherein the phenyl or heteroaryl is optionally substituted with 1-3 substituents selected from halo, C₁-C₃ alkyl, C₁-C₃ alkoxy, —CN, and C₁-C₃ haloalkyl.

In some embodiments the inhibitor of POL Q is a compound of formula (I), or any stereoisomer thereof or pharmaceutically acceptable salt thereof, wherein Y is phenyl.

In some embodiments the inhibitor of POL Q is a compound of formula (I), or any stereoisomer thereof or pharmaceutically acceptable salt thereof, wherein Y is a N-heteroaryl.

In some embodiments the inhibitor of POL Q is a compound of formula (I), or any stereoisomer thereof or pharmaceutically acceptable salt thereof, wherein Y is a pyridine.

In some embodiments the inhibitor of POL Q is a compound of formula (I), or any stereoisomer thereof or pharmaceutically acceptable salt thereof, wherein Y is substituted.

In some embodiments the inhibitor of POL Q is a compound of formula (I), or any stereoisomer thereof or pharmaceutically acceptable salt thereof, wherein Y is substituted with —Cl.

In some embodiments the inhibitor of POL Q is a compound of formula (I), or any stereoisomer thereof or pharmaceutically acceptable salt thereof, wherein Y is substituted with —CH₃.

In some embodiments the inhibitor of POL Q is a compound of formula (I), or any stereoisomer thereof or pharmaceutically acceptable salt thereof, wherein Y is not substituted.

In some embodiments the inhibitor of POL Q is a compound of formula (I), or any stereoisomer thereof or pharmaceutically acceptable salt thereof, wherein R¹ is -halo.

In some embodiments the inhibitor of POL Q is a compound of formula (I), or any stereoisomer thereof or pharmaceutically acceptable salt thereof, wherein R¹ is —Cl.

In some embodiments the inhibitor of POL Q is a compound of formula (I), or any stereoisomer thereof or pharmaceutically acceptable salt thereof, wherein R¹ is —F.

In some embodiments the inhibitor of POL Q is a compound of formula (I), or any stereoisomer thereof or pharmaceutically acceptable salt thereof, wherein R¹ is —CH₃.

In some embodiments the inhibitor of POL Q is a compound of formula (I), or any stereoisomer thereof or pharmaceutically acceptable salt thereof, wherein R² is —H.

In some embodiments the inhibitor of POL Q is a compound of formula (I), or any stereoisomer thereof or pharmaceutically acceptable salt thereof, wherein Q¹, Q², or Q³ is C-L-R.

In some embodiments the inhibitor of POL Q is a compound of formula (I), or any stereoisomer thereof or pharmaceutically acceptable salt thereof, wherein Q¹ is C-L-R.

In some embodiments the inhibitor of POL Q is a compound of formula (I), or any stereoisomer thereof or pharmaceutically acceptable salt thereof, wherein Q² is C-L-R.

In some embodiments the inhibitor of POL Q is a compound of formula (I), or any stereoisomer thereof or pharmaceutically acceptable salt thereof, wherein Q³ is C-L-R.

In some embodiments the inhibitor of POL Q is a compound of formula (I), or any stereoisomer thereof or pharmaceutically acceptable salt thereof, wherein Q¹, Q², or Q³ is C-L-R and L is a bond, —O—, —(CH₂)ₚO— or —O(CH₂)ₚ—.

In some embodiments the inhibitor of POL Q is a compound of formula (I), or any stereoisomer thereof or pharmaceutically acceptable salt thereof, wherein Q¹ is C-L-R and L is a bond, —O—, —(CH₂)ₚO— or —O(CH₂)ₚ—.

In some embodiments the inhibitor of POL Q is a compound of formula (I), or any stereoisomer thereof or pharmaceutically acceptable salt thereof, wherein Q² is C-L-R and L is a bond, —O—, —(CH₂)ₚO— or —O(CH₂)ₚ—.

In some embodiments the inhibitor of POL Q is a compound of formula (I), or any stereoisomer thereof or pharmaceutically acceptable salt thereof, wherein Q³ is C-L-R and L is a bond, —O—, —(CH₂)ₚO— or —O(CH₂)ₚ—.

In some embodiments the inhibitor of POL Q is a compound of formula (I), or any stereoisomer thereof or pharmaceutically acceptable salt thereof, wherein Q¹, Q², or Q³ is C-L-R and L is a bond.

In some embodiments the inhibitor of POL Q is a compound of formula (I), or any stereoisomer thereof or pharmaceutically acceptable salt thereof, wherein Q¹ is C-L-R and L is a bond.

In some embodiments the inhibitor of POL Q is a compound of formula (I), or any stereoisomer thereof or pharmaceutically acceptable salt thereof, wherein Q² is C-L-R and L is a bond.

In some embodiments the inhibitor of POL Q is a compound of formula (I), or any stereoisomer thereof or pharmaceutically acceptable salt thereof, wherein Q³ is C-L-R and L is a bond.

In some embodiments the inhibitor of POL Q is a compound of formula (I), or any stereoisomer thereof or pharmaceutically acceptable salt thereof, wherein Q¹, Q², or Q³ is C-L-R and L is —O—.

In some embodiments the inhibitor of POL Q is a compound of formula (I), or any stereoisomer thereof or pharmaceutically acceptable salt thereof, wherein Q¹ is C-L-R and L is—O—.

In some embodiments the inhibitor of POL Q is a compound of formula (I), or any stereoisomer thereof or pharmaceutically acceptable salt thereof, wherein Q² is C-L-R and L is—O—.

In some embodiments the inhibitor of POL Q is a compound of formula (I), or any stereoisomer thereof or pharmaceutically acceptable salt thereof, wherein Q³ is C-L-R and L is—O—.

In some embodiments the inhibitor of POL Q is a compound of formula (I), or any stereoisomer thereof or pharmaceutically acceptable salt thereof, wherein Q¹, Q², or Q³ is C-L-R and L is —(CH₂)ₚO.

In some embodiments the inhibitor of POL Q is a compound of formula (I), or any stereoisomer thereof or pharmaceutically acceptable salt thereof, wherein Q¹ is C-L-R and L is —(CH₂)ₚO.

In some embodiments the inhibitor of POL Q is a compound of formula (I), or any stereoisomer thereof or pharmaceutically acceptable salt thereof, wherein Q² is C-L-R and L is —(CH₂)ₚO.

In some embodiments the inhibitor of POL Q is a compound of formula (I), or any stereoisomer thereof or pharmaceutically acceptable salt thereof, wherein Q³ is C-L-R and L is —(CH₂)ₚO.

In some embodiments the inhibitor of POL Q is a compound of formula (I), or any stereoisomer thereof or pharmaceutically acceptable salt thereof, wherein Q¹, Q², or Q³ is C-L-R and L is —O(CH₂)ₚ—.

In some embodiments the inhibitor of POL Q is a compound of formula (I), or any stereoisomer thereof or pharmaceutically acceptable salt thereof, wherein Q¹ is C-L-R and L is —O(CH₂)ₚ—.

In some embodiments the inhibitor of POL Q is a compound of formula (I), or any stereoisomer thereof or pharmaceutically acceptable salt thereof, wherein Q² is C-L-R and L is —O(CH₂)ₚ—.

In some embodiments the inhibitor of POL Q is a compound of formula (I), or any stereoisomer thereof or pharmaceutically acceptable salt thereof, wherein Q³ is C-L-R and L is —O(CH₂)ₚ—.

In some embodiments the inhibitor of POL Q is a compound of formula (I), or any stereoisomer thereof or pharmaceutically acceptable salt thereof, wherein Q¹, Q², or Q³ is C-L-R and L is —O(CH₂)₂—.

In some embodiments the inhibitor of POL Q is a compound of formula (I), or any stereoisomer thereof or pharmaceutically acceptable salt thereof, wherein Q¹ is C-L-R and L is —O(CH₂)₂—.

In some embodiments the inhibitor of POL Q is a compound of formula (I), or any stereoisomer thereof or pharmaceutically acceptable salt thereof, wherein Q² is C-L-R and L is —O(CH₂)₂—.

In some embodiments the inhibitor of POL Q is a compound of formula (I), or any stereoisomer thereof or pharmaceutically acceptable salt thereof, wherein Q³ is C-L-R and L is —O(CH₂)₂—.

In some embodiments the inhibitor of POL Q is a compound of formula (I), or any stereoisomer thereof or pharmaceutically acceptable salt thereof, wherein Q¹, Q², or Q³ is C-L-R and L is —C(O)—; —O(CH₂)ₚC(O)—; —C(O)NR^{y}—; —O(CH₂)ₚC(O)NR^{y}—; —(CH₂)ₚC(O)—; or —(CH₂)ₚC(O)O—.

In some embodiments the inhibitor of POL Q is a compound of formula (I), or any stereoisomer thereof or pharmaceutically acceptable salt thereof, wherein Q¹ is C-L-R and L is —C(O)—; —O(CH₂)ₚC(O)—; —C(O)NR^{y}—; —O(CH₂)ₚC(O)NR^{y}—; —(CH₂)ₚC(O)—; or —(CH₂)ₚC(O)O—.

In some embodiments the inhibitor of POL Q is a compound of formula (I), or any stereoisomer thereof or pharmaceutically acceptable salt thereof, wherein Q² is C-L-R and L is —C(O)—; —O(CH₂)ₚC(O)—; —C(O)NR^{y}—; —O(CH₂)ₚC(O)NR^{y}—; —(CH₂)ₚC(O)—; or —(CH₂)ₚC(O)O—.

In some embodiments the inhibitor of POL Q is a compound of formula (I), or any stereoisomer thereof or pharmaceutically acceptable salt thereof, wherein Q³ is C-L-R and L is —C(O)—; —O(CH₂)ₚC(O)—; —C(O)NR^{y}—; —O(CH₂)ₚC(O)NR^{y}—; —(CH₂)ₚC(O)—; or —(CH₂)ₚC(O)O—.

In some embodiments the inhibitor of POL Q is a compound of formula (I), or any stereoisomer thereof or pharmaceutically acceptable salt thereof, wherein Q¹, Q², or Q³ is C-L-R and R is H, R^{a} or R^{b}.

In some embodiments the inhibitor of POL Q is a compound of formula (I), or any stereoisomer thereof or pharmaceutically acceptable salt thereof, wherein Q¹ is C-L-R and R is H, R^{a} or R^{b}.

In some embodiments the inhibitor of POL Q is a compound of formula (I), or any stereoisomer thereof or pharmaceutically acceptable salt thereof, wherein Q² is C-L-R and R is H, R^{a} or R^{b}.

In some embodiments the inhibitor of POL Q is a compound of formula (I), or any stereoisomer thereof or pharmaceutically acceptable salt thereof, wherein Q³ is C-L-R and R is H, R^{a} or R^{b}.

In some embodiments the inhibitor of POL Q is a compound of formula (I), or any stereoisomer thereof or pharmaceutically acceptable salt thereof, wherein Q¹, Q², or Q³ is C-L-R and R is R^{a}.

In some embodiments the inhibitor of POL Q is a compound of formula (I), or any stereoisomer thereof or pharmaceutically acceptable salt thereof, wherein Q¹, Q², or Q³ is C-L-R^{a} and R^{a} is a 3-10 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and R^{a} is a 4-7 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and R^{a} is a 6 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and R^{a} is piperidine, 1,2-diazinane, 1,3-diazinane, 1,4-diazinane, 1,2-oxazinane, 1,3-oxazinane, 1,4-oxazinane.

In some embodiments the inhibitor of POL Q is a compound of formula (I), or any stereoisomer thereof or pharmaceutically acceptable salt thereof, wherein Q¹, Q², or Q³ is C-L-R^{a} and R^{a} is substituted with 1 to 4 substituents selected from amino, carboxy, halo, hydroxy, oxo, -CN, —S(O)₂OH, C₁-C₄ alkylamino, C₁-C₅ alkoxy, C₂-C₅ alkoxyalkyl, 4-6 membered heterocycle, and C₁-C₇ alkyl, wherein the C₁-C₇ alkyl is optionally substituted with 1 to 4 substituents selected from amino, carboxy, halo, hydroxy, oxo, —CN, C₂-C₈ ester, and C₁-C₅ alkoxy.

In some embodiments the inhibitor of POL Q is a compound of formula (I), or any stereoisomer thereof or pharmaceutically acceptable salt thereof, wherein Q¹, Q², or Q³ is C-L-R^{a} and R^{a} is substituted with C₁-C₇ alkyl, wherein the C₁-C₇ alkyl is optionally substituted with 1 to 4 substituents selected from amino, carboxy, halo, hydroxy, oxo, —CN, C₂-C₈ ester, and C₁-C₅ alkoxy.

In some embodiments the inhibitor of POL Q is a compound of formula (I), or any stereoisomer thereof or pharmaceutically acceptable salt thereof, wherein Q¹, Q², or Q³ is C-L-R^{a} and R^{a} is substituted with C₁-C₇ alkyl substituted with oxo.

In some embodiments the inhibitor of POL Q is a compound of formula (I), or any stereoisomer thereof or pharmaceutically acceptable salt thereof, wherein Q¹, Q², or Q³ is C-L-R^{a} and R^{a} is substituted with C₁-C₇ alkyl substituted with a C₁-C₅ alkoxy.

In some embodiments the inhibitor of POL Q is a compound of formula (I), or any stereoisomer thereof or pharmaceutically acceptable salt thereof, wherein Q¹, Q², or Q³ is C-L-R^{a} and R^{a} is substituted with methyl.

In some embodiments the inhibitor of POL Q is a compound of formula (I), or any stereoisomer thereof or pharmaceutically acceptable salt thereof, wherein Q¹, Q², or Q³ is C-L-R^{a} and L is —O(CH₂)ₚ— and R^{a} is an optionally substituted 3-10 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is —O(CH₂)ₚ— and R^{a} is an optionally substituted 3-10 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is —O(CH₂)₂— and R^{a} is an optionally substituted 3-10 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is —O(CH₂)₂— and R^{a} is an optionally substituted 4-7 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is —O(CH₂)₂— and R^{a} is an unsubstituted 4-7 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is —O(CH₂)₂— and R^{a} is a 4-7 membered N-heterocycle substituted with hydroxy, methyl, or amino.

In some embodiments the inhibitor of POL Q is a compound of formula (I), or any stereoisomer thereof or pharmaceutically acceptable salt thereof, wherein Q¹, Q², or Q³ is C-L-R^{a} and L is —O(CH₂)₃—and R^{a} is an optionally substituted 3-10 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is —O(CH₂)₃— and R^{a} is a 4-7 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is —O(CH₂)₃— and R^{a} is an unsubstituted 4-7 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is —O(CH₂)₃— and R^{a} is a 4-7 membered N-heterocycle substituted with hydroxy, methyl, or amino.

In some embodiments the inhibitor of POL Q is a compound of formula (I), or any stereoisomer thereof or pharmaceutically acceptable salt thereof, wherein Q¹, Q², or Q³ is C-L-R^{a} and L is a bond and R^{a} is an optionally substituted 3-10 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is a bond and R^{a} is a 4-7 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is a bond and R^{a} is an unsubstituted 4-7 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is a bond and R^{a} is a 4-7 membered N-heterocycle substituted with hydroxy, methyl, or amino.

In some embodiments the inhibitor of POL Q is a compound of formula (I), or any stereoisomer thereof or pharmaceutically acceptable salt thereof, wherein Q¹, Q², or Q³ is C-L-R^{a} and L is —O— and R^{a} is an optionally substituted 3-10 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is —O— and R^{a} is a 4-7 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is —O— and R^{a} is an unsubstituted 4-7 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{a} and L is —O— and R^{a} is a 4-7 membered N-heterocycle substituted with hydroxy, methyl, or amino.

In some embodiments the inhibitor of POL Q is a compound of formula (I), or any stereoisomer thereof or pharmaceutically acceptable salt thereof, wherein Q¹, Q², or Q³ is C-L-R and R is R^{b}.

In some embodiments the inhibitor of POL Q is a compound of formula (I), or any stereoisomer thereof or pharmaceutically acceptable salt thereof, wherein Q¹, Q², or Q³ is C-L-R^{b} and R^{b} is substituted with 1 to 4 substituents selected from: halo, oxo, hydroxy, carboxyl, amino, —CN, C₂-C₄ alkynyl, C₂-C₆ carbamate, C₁-C₈ amide, C₁-C₄ sulfonyl, C₁-C₄ sulfonamide, C₁-C₄ alkylamino, C₁-C₅ alkoxy, C₃-C₆ carbocycle, and 3-10 membered heterocycle, wherein the C₃-C₆ carbocycle is optionally substituted with 1 to 4 substituents selected from hydroxy, halo, and carboxy, and wherein the 3-10 membered heterocycle is optionally substituted with 1 to 4 substituents selected from amino, carboxy, halo, hydroxy, oxo, —CN, —S(O)₂OH, C₁-C₄ alkylamino, C₁-C₅ alkoxy, C₂-C₅ alkoxyalkyl, 4-6 membered heterocycle, and C₁-C₇ alkyl, wherein the C₁-C₇ alkyl is optionally substituted with 1 to 4 substituents selected from amino, carboxy, halo, hydroxy, oxo, —CN, C₂-C₈ ester, and C₁-C₅ alkoxy.

In some embodiments the inhibitor of POL Q is a compound of formula (I), or any stereoisomer thereof or pharmaceutically acceptable salt thereof, wherein Q¹, Q², or Q³ is C-L-R^{b} and R^{b} is substituted with at least one C₃-C₆ carbocycle, wherein the C₃-C₆ carbocycle is substituted with 1 to 4 substituents selected from hydroxy, halo, and carboxy.

In some embodiments the inhibitor of POL Q is a compound of formula (I), or any stereoisomer thereof or pharmaceutically acceptable salt thereof, wherein Q¹, Q², or Q³ is C-L-R^{b} and R^{b} is substituted with at least one C₃-C₆ carbocycle, wherein the C₃-C₆ carbocycle is substituted with 1 to 4 substituents selected from hydroxy and halo.

In some embodiments the inhibitor of POL Q is a compound of formula (I), or any stereoisomer thereof or pharmaceutically acceptable salt thereof, wherein Q¹, Q², or Q³ is C-L-R^{b} and R^{b} is substituted with hydroxy.

In some embodiments the inhibitor of POL Q is a compound of formula (I), or any stereoisomer thereof or pharmaceutically acceptable salt thereof, wherein Q¹, Q², or Q³ is C-L-R^{b} and R^{b} is substituted with a 3-10 membered heterocycle.

In some embodiments the inhibitor of POL Q is a compound of formula (I), or any stereoisomer thereof or pharmaceutically acceptable salt thereof, wherein Q¹, Q², or Q³ is C-L-R^{b} and R^{b} is substituted with a N-heterocycle.

In some embodiments the inhibitor of POL Q is a compound of formula (I), or any stereoisomer thereof or pharmaceutically acceptable salt thereof, wherein Q¹, Q², or Q³ is C-L-R^{b} and R^{b} is substituted with a 4-7 membered N-heterocycle.

In some embodiments the inhibitor of POL Q is a compound of formula (I), or any stereoisomer thereof or pharmaceutically acceptable salt thereof, wherein Q¹, Q², or Q³ is C-L-R^{b} and R^{b} is substituted with a 3-10 membered heterocycle, such as, but not limited to a N-heterocycle, such as, but not limited to a 4-7 membered N-heterocycle, wherein the heterocycle is substituted with 1 to 4 substituents selected from amino, carboxy, halo, hydroxy, oxo, —CN, —S(O)₂OH, C1-C4 alkylamino, C1-C5 alkoxy, C₂-C₅ alkoxyalkyl, 4-6 membered heterocycle, and C₁-C₇ alkyl, wherein the C1-C7 alkyl is optionally substituted with 1 to 4 substituents selected from amino, carboxy, halo, hydroxy, oxo, —CN, C₂-C₈ ester, and C₁-C₅ alkoxy.

In some embodiments the inhibitor of POL Q is a compound of formula (I), or any stereoisomer thereof or pharmaceutically acceptable salt thereof, wherein Q¹, Q², or Q³ is C-L-R^{b} and R^{b} is substituted with a 3-10 membered heterocycle, such as, but not limited to a N-heterocycle, such as, but not limited to a 4-7 membered N-heterocycle, wherein the heterocycle is substituted with C1-C7 alkyl, oxo, and/or halo.

In some embodiments the inhibitor of POL Q is a compound of formula (I), or any stereoisomer thereof or pharmaceutically acceptable salt thereof, wherein Q¹, Q², or Q³ is C-L-R^{b} and R^{b} is substituted with amino, C₁-C₈ amide, and/or C₁-C₄ alkylamino.

In some embodiments the inhibitor of POL Q is a compound of formula (I), or any stereoisomer thereof or pharmaceutically acceptable salt thereof, wherein Q¹, Q², or Q³ is C-L-R^{b} and R^{b} is substituted with oxo, hydroxy, and/or carboxy.

In some embodiments the inhibitor of POL Q is a compound of formula (I), or any stereoisomer thereof or pharmaceutically acceptable salt thereof, wherein Q¹, Q², or Q³ is C-L-R^{b}L is —O—, and R^{b} is an optionally substituted C₁-C₅ alkyl.

In some embodiments the inhibitor of POL Q is a compound of formula (I), or any stereoisomer thereof or pharmaceutically acceptable salt thereof, wherein Q¹, Q², or Q³ is C-L-R^{b}, L is —O—, and R^{b} is an optionally substituted C₁-C₃ alkyl.

In some embodiments the inhibitor of POL Q is a compound of formula (I), or any stereoisomer thereof or pharmaceutically acceptable salt thereof, wherein Q¹, Q², or Q³ is C-L-R^{b}, L is —O—, and R^{b} is an C₁-C₅ alkyl substituted with 1 to 4 substituents selected from amino, carboxy, oxy, and hydroxy.

In some embodiments the inhibitor of POL Q is a compound of formula (I), or any stereoisomer thereof or pharmaceutically acceptable salt thereof, wherein Q¹, Q², or Q³ is C-L-R^{b}, L is —O—, and R^{b} is an C₁-C₃ alkyl substituted with 1 to 4 substituents selected from amino, carboxy, oxy, and hydroxy.

In some embodiments the inhibitor of POL Q is a compound of formula (I), or any stereoisomer thereof or pharmaceutically acceptable salt thereof, wherein Q¹, Q², or Q³ is C-L-R^{b}, L is —O—, and R^{b} is an C₁-C₅ alkyl substituted with 1 to 4 substituents selected from —CN, C₂-C₄ alkynyl, C₂-C₆ carbamate, and C₁-C₈ amide.

In some embodiments the inhibitor of POL Q is a compound of formula (I), or any stereoisomer thereof or pharmaceutically acceptable salt thereof, wherein Q¹, Q², or Q³ is C-L-R^{b}, L is —O—, and R^{b} is an C₁-C₃ alkyl substituted with 1 to 4 substituents selected from —CN, C₂-C₄ alkynyl, C₂-C₆ carbamate, and C₁-C₈ amide.

In some embodiments the inhibitor of POL Q is a compound of formula (I), or any stereoisomer thereof or pharmaceutically acceptable salt thereof, wherein Q¹, Q², or Q³ is C-L-R^{b}L is —O—, and R^{b} is an unsubstituted C₁-C₅ alkyl.

In some embodiments the inhibitor of POL Q is a compound of formula (I), or any stereoisomer thereof or pharmaceutically acceptable salt thereof, wherein Q¹, Q², or Q³ is C-L-R^{b}L is —O—, and R^{b} is an unsubstituted C₁-C₃ alkyl.

In some embodiments the inhibitor of POL Q is a compound of formula (I), or any stereoisomer thereof or pharmaceutically acceptable salt thereof, wherein Q¹, Q², or Q³ is C-L-R^{b}, L is —O—, and R^{b} is an optionally substituted C₁-C₇ alkyl, wherein one or two methylene groups from the C₁-C₇ alkyl is replaced with NR^{e} or O.

In some embodiments the inhibitor of POL Q is a compound of formula (I), or any stereoisomer thereof or pharmaceutically acceptable salt thereof, wherein Q¹, Q², or Q³ is C-L-R^{b}, L is —O—, and R^{b} is an optionally substituted C₁-C₇ alkyl, wherein one methylene group from the C₁-C₇ alkyl is replaced with NH.

In some embodiments the inhibitor of POL Q is a compound of formula (I), or any stereoisomer thereof or pharmaceutically acceptable salt thereof, wherein Q¹, Q², or Q³ is C-L-R^{b}, L is —O—, and R^{b} is an optionally substituted C₁-C₇ alkyl, wherein one methylene group from the C₁-C₇ alkyl is replaced with NCH₃.

In some embodiments the inhibitor of POL Q is a compound of formula (I), or any stereoisomer thereof or pharmaceutically acceptable salt thereof, wherein Q¹, Q², or Q³ is C-L-R^{b}L is a bond, and R^{b} is an optionally substituted C₁-C₅ alkyl.

In some embodiments the inhibitor of POL Q is a compound of formula (I), or any stereoisomer thereof or pharmaceutically acceptable salt thereof, wherein Q¹, Q², or Q³ is C-L-R^{b}L is a bond, and R^{b} is an optionally substituted C₁-C₃ alkyl.

In some embodiments the inhibitor of POL Q is a compound of formula (I), or any stereoisomer thereof or pharmaceutically acceptable salt thereof, wherein Q¹, Q², or Q³ is C-L-R^{b}, L is a bond, and R^{b} is an C₁-C₅ alkyl substituted with 1 to 4 substituents selected from amino, carboxy, oxy, and hydroxy.

In some embodiments the inhibitor of POL Q is a compound of formula (I), or any stereoisomer thereof or pharmaceutically acceptable salt thereof, wherein Q¹, Q², or Q³ is C-L-R^{b}, L is a bond, and R^{b} is an C₁-C₃ alkyl substituted with 1 to 4 substituents selected from amino, carboxy, oxy, and hydroxy.

In some embodiments the inhibitor of POL Q is a compound of formula (I), or any stereoisomer thereof or pharmaceutically acceptable salt thereof, wherein Q¹, Q², or Q³ is C-L-R^{b}, L is a bond, and R^{b} is an C₁-C₅ alkyl substituted with 1 to 4 substituents selected from —CN, C₂-C₄ alkynyl, C₂-C₆ carbamate, and C₁-C₈ amide.

In some embodiments the inhibitor of POL Q is a compound of formula (I), or any stereoisomer thereof or pharmaceutically acceptable salt thereof, wherein Q¹, Q², or Q³ is C-L-R^{b}, L is a bond, and R^{b} is an C₁-C₃ alkyl substituted with 1 to 4 substituents selected from —CN, C₂-C₄ alkynyl, C₂-C₆ carbamate, and C₁-C₈ amide.

In some embodiments the inhibitor of POL Q is a compound of formula (I), or any stereoisomer thereof or pharmaceutically acceptable salt thereof, wherein Q¹, Q², or Q³ is C-L-R^{b}L is a bond, and R^{b} is an unsubstituted C₁-C₅ alkyl.

In some embodiments the inhibitor of POL Q is a compound of formula (I), or any stereoisomer thereof or pharmaceutically acceptable salt thereof, wherein Q¹, Q², or Q³ is C-L-R^{b}L is a bond, and R^{b} is an unsubstituted C₁-C₃ alkyl.

In some embodiments the inhibitor of POL Q is a compound of formula (I), or any stereoisomer thereof or pharmaceutically acceptable salt thereof, wherein Q¹, Q², or Q³ is C-L-R^{b}, L is a bond, and R^{b} is an optionally substituted C₁-C₇ alkyl, wherein one or two methylene groups from the C₁-C₇ alkyl is replaced with NR^{e} or O.

In some embodiments the inhibitor of POL Q is a compound of formula (I), or any stereoisomer thereof or pharmaceutically acceptable salt thereof, wherein Q¹, Q², or Q³ is C-L-R^{b}, L is a bond, and R^{b} is an optionally substituted C₁-C₇ alkyl, wherein one methylene group from the C₁-C₇ alkyl is replaced with NH.

In some embodiments the inhibitor of POL Q is a compound of formula (I), or any stereoisomer thereof or pharmaceutically acceptable salt thereof, wherein Q¹, Q², or Q³ is C-L-R^{b}, L is a bond, and R^{b} is an optionally substituted C₁-C₇ alkyl, wherein one methylene group from the C₁-C₇ alkyl is replaced with NCH₃.

In some embodiments the inhibitor of POL Q is a compound of formula (I), or any stereoisomer thereof or pharmaceutically acceptable salt thereof, wherein Q¹, Q², or Q³ is C-L-R^{c}—.

In some embodiments the inhibitor of POL Q is a compound of formula (I), or any stereoisomer thereof or pharmaceutically acceptable salt thereof, wherein Q¹, Q², or Q³ is C-L-R^{c} and R^{c} is substituted with 1 to 4 substituents selected from hydroxy, halo, and carboxy.

In some embodiments the inhibitor of POL Q is a compound of formula (I), or any stereoisomer thereof or pharmaceutically acceptable salt thereof, wherein Q¹, Q², or Q³is C-L-R and R^{c} is substituted with 1 to 4 substituents selected from hydroxy and halo.

In some embodiments, the inhibitor of POL Q is a compound listed in Table I, a pharmaceutically acceptable salt thereof, or combinations thereof.

In some embodiments, the inhibitor of POL Q is a compound listed in Table I, Table II, Table III, a pharmaceutically acceptable salt thereof, or combinations thereof.

TABLE I
4-(1-benzyl-5-isopropoxy-1H-benzo[d]imidazol-2-yl)-3-chlorophenol,
tert-butyl (3-(4-(1-benzyl-5-isopropoxy-1H-benzo[d]imidazol-2-yl)-3-
chlorophenoxy)propyl)carbamate,
N-(3-(4-(1-benzyl-5-isopropoxy-1H-benzo[d]imidazol-2-yl)-3-
chlorophenoxy)propyl)acetamide,
N-(3-(4-(1-benzyl-5-isopropoxy-1H-benzo[d]imidazol-2-yl)-3-
chlorophenoxy)propyl)heptanamide,
1-(4-chlorobenzyl)-5-isopropoxy-2-(4-methyl-6-(2-(4-methylpiperazin-1-yl)ethoxy)pyridin-3-
yl)-1H-benzo[d]imidazole,
2-(2-chloro-4-(2-(4-methylpiperazin-1-yl)ethoxy)phenyl)-1-(2-chloro-6-fluoro-3-
methylbenzyl)-5-isopropoxy-1H-benzo[d]imidazole,
1-(3-chlorobenzyl)-5-isopropoxy-2-(4-methyl-6-(2-(4-methylpiperazin-1-yl)ethoxy)pyridin-3-
yl)-1H-benzo[d]imidazole,
1-benzyl-2-(2-chloro-4-(2-(1-methylpyrrolidin-2-yl)ethoxy)phenyl)-5-isopropoxy-1H-
benzo[d]imidazole (Isomer 1),
1-benzyl-2-(2-chloro-4-(2-(1-methylpyrrolidin-2-yl)ethoxy)phenyl)-5-isopropoxy-1H-
benzo[d]imidazole (Isomer 2),
(R)-1-benzyl-2-(2-chloro-4-(2-(1-methylpyrrolidin-2-yl)ethoxy)phenyl)-5-isopropoxy-1H-
benzo[d]imidazole,
(S)-1-benzyl-2-(2-chloro-4-(2-(1-methylpyrrolidin-2-yl)ethoxy)phenyl)-5-isopropoxy-1H-
benzo[d]imidazole,
1-benzyl-2-(2-chloro-4-((1-methylazepan-4-yl)oxy)phenyl)-5-isopropoxy-1H-
benzo[d]imidazole (Isomer 1),
1-benzyl-2-(2-chloro-4-((1-methylazepan-4-yl)oxy)phenyl)-5-isopropoxy-1H-
benzo[d]imidazole (Isomer 2),
(R)-1-benzyl-2-(2-chloro-4-((1-methylazepan-4-yl)oxy)phenyl)-5-isopropoxy-1H-
benzo[d]imidazole,
(S)-1-benzyl-2-(2-chloro-4-((1-methylazepan-4-yl)oxy)phenyl)-5-isopropoxy-1H-
benzo[d]imidazole
1-benzyl-2-(2-chloro-4-methoxyphenyl)-5-isopropoxy-1H-benzo[d]imidazole,
9-benzyl-6-isopropoxy-8-(4-(2-(4-methylpiperazin-1-yl)ethoxy)phenyl)-9H-purine,
9-benzyl-6-isopropoxy-8-(2-methyl-4-(2-(piperazin-1-yl)ethoxy)phenyl)-9H-purine,
tert-butyl 4-(2-(4-(9-benzyl-6-isopropoxy-9H-purin-8-yl)-3-methylphenoxy)ethyl)piperazine-
1-carboxylate,
1-(4-(2-(4-(9-benzyl-6-isopropoxy-9H-purin-8-yl)-3-methylphenoxy)ethyl)piperazin-1-
yl)ethan-1-one,
1-(4-(2-(4-(9-benzyl-6-isopropoxy-9H-purin-8-yl)-3-methylphenoxy)ethyl)piperazin-1-
yl)hexan-1-one,
9-benzyl-8-(2-methyl-4-(2-(4-methylpiperazin-1-yl)ethoxy)phenyl)-6-(1-
methylcyclopropoxy)-9H-purine,
1-(4-(5-(9-benzyl-6-(1-methylcyclopropoxy)-9H-purin-8-yl)-4-methylpyridin-2-yl)piperazin-
1-yl)ethan-1-one,
9-benzyl-8-(4-methyl-6-(2-(4-methylpiperazin-1-yl)ethoxy)pyridin-3-yl)-6-(1-
methylcyclopropoxy)-9H-purine,
(R)-1-(4-(9-benzyl-6-(1-methylcyclopropoxy)-9H-purin-8-yl)-3-chlorophenyl)-N,N-
dimethylpyrrolidin-3-amine,
9-benzyl-8-(4-methyl-6-(piperazin-1-yl)pyridin-3-yl)-6-(1-methylcyclopropoxy)-9H-purine,
9-benzyl-8-(2-chloro-3-methoxyphenyl)-6-(1-methylcyclopropoxy)-9H-purine,
9-benzyl-8-(2-chloro-4-(piperazin-1-yl)phenyl)-6-(1-methylcyclopropoxy)-9H-purine,
2-(4-(9-benzyl-6-(1-methylcyclopropoxy)-9H-purin-8-yl)-3-chlorophenoxy)-1-(piperazin-1-
yl)ethan-1-one,
2-(4-(9-benzyl-6-(1-methylcyclopropoxy)-9H-purin-8-yl)-3-chlorophenoxy)-1-(4-
methylpiperazin-1-yl)ethan-1-one,
1-(4-((4-(9-benzyl-6-(1-methylcyclopropoxy)-9H-purin-8-yl)-3-
chlorophenoxy)methyl)piperidin-1-yl)ethan-1-one,
9-benzyl-8-(2-chloro-4-(2-(1-methylpyrrolidin-2-yl)ethoxy)phenyl)-6-(1-
methylcyclopropoxy)-9H-purine,
9-benzyl-8-(2-chloro-4-(2-(1-methylpyrrolidin-2-yl)ethoxy)phenyl)-6-(1-
methylcyclopropoxy)-9H-purine (Isomer 2),
9-benzyl-8-(2-chloro-4-(2-(1-methylpyrrolidin-2-yl)ethoxy)phenyl)-6-(1-
methylcyclopropoxy)-9H-purine (Isomer 1),
(R)-9-benzyl-8-(2-chloro-4-(2-(1-methylpyrrolidin-2-yl)ethoxy)phenyl)-6-(1-
methylcyclopropoxy)-9H-purine,
(S)-9-benzyl-8-(2-chloro-4-(2-(1-methylpyrrolidin-2-yl)ethoxy)phenyl)-6-(1-
methylcyclopropoxy)-9H-purine,
1-(4-(2-(4-(9-benzyl-6-(1-methylcyclopropoxy)-9H-purin-8-yl)-3-
chlorophenoxy)ethyl)piperazin-1-yl)ethan-1-one,
3-(4-(9-benzyl-6-(1-methylcyclopropoxy)-9H-purin-8-yl)-3-chlorophenoxy)propan-1-amine,
N-(3-(4-(9-benzyl-6-(1-methylcyclopropoxy)-9H-purin-8-yl)-3-
chlorophenoxy)propyl)acetamide,
1-(4-(2-(3-(9-benzyl-6-(1-methylcyclopropoxy)-9H-purin-8-yl)-2-
chlorophenoxy)ethyl)piperazin-1-yl)ethan-1-one,
9-benzyl-8-(2-chloro-3-((1-methylpiperidin-4-yl)oxy)phenyl)-6-(1-methylcyclopropoxy)-9H-
purine,
9-benzyl-8-(2-chloro-3-(2-(piperazin-1-yl)ethoxy)phenyl)-6-(1-methylcyclopropoxy)-9H-
purine,
9-benzyl-8-(2-chloro-3-(piperidin-4-yloxy)phenyl)-6-(1-methylcyclopropoxy)-9H-purine,
9-benzyl-8-(2-chloro-3-(2-(4-methylpiperazin-1-yl)ethoxy)phenyl)-6-(1-methylcyclopropoxy)-
9H-purine,
9-benzyl-8-(2-chloro-4-((1-methylazepan-4-yl)oxy)phenyl)-6-(1-methylcyclopropoxy)-9H-
purine (Isomer 1),
9-benzyl-8-(2-chloro-4-((1-methylazepan-4-yl)oxy)phenyl)-6-(1-methylcyclopropoxy)-9H-
purine (Isomer 2),
(R)-9-benzyl-8-(2-chloro-4-((1-methylazepan-4-yl)oxy)phenyl)-6-(1-methylcyclopropoxy)-
9H-purine,
(S)-9-benzyl-8-(2-chloro-4-((1-methylazepan-4-yl)oxy)phenyl)-6-(1-methylcyclopropoxy)-
9H-purine,
4-(2-(4-(9-benzyl-6-(1-methylcyclopropoxy)-9H-purin-8-yl)-3-
chlorophenoxy)ethyl)piperazin-2-one,
(R)-8-(4-(azetidin-2-ylmethoxy)-2-chlorophenyl)-9-benzyl-6-(1-methylcyclopropoxy)-9H-
purine,
(S)-9-benzyl-8-(2-chloro-4-(pyrrolidin-3-yloxy)phenyl)-6-(1-methylcyclopropoxy)-9H-purine
9-benzyl-8-(2-chloro-4-(3-(piperazin-1-yl)propoxy)phenyl)-6-(1-methylcyclopropoxy)-9H-
purine,
9-benzyl-8-(2-chloro-4-((3-fluoroazetidin-3-yl)methoxy)phenyl)-6-(1-methylcyclopropoxy)-
9H-purine,
2-(4-(9-benzyl-6-(1-methylcyclopropoxy)-9H-purin-8-yl)-3-chlorophenoxy)-N,N-
dimethylacetamide,
2-(4-(9-benzyl-6-(1-methylcyclopropoxy)-9H-purin-8-yl)-3-chlorophenoxy)-N-
methylacetamide,
1-(4-(3-(4-(9-benzyl-6-(1-methylcyclopropoxy)-9H-purin-8-yl)-3-
chlorophenoxy)propyl)piperazin-1-yl)ethan-1-one,
2-(4-(9-benzyl-6-(1-methylcyclopropoxy)-9H-purin-8-yl)-3-chlorophenoxy)ethan-1-amine,
2-(4-(9-benzyl-6-(1-methylcyclopropoxy)-9H-purin-8-yl)-3-chlorophenoxy)acetic acid,
(R)-9-benzyl-8-(2-chloro-4-(pyrrolidin-2-ylmethoxy)phenyl)-6-(1-methylcyclopropoxy)-9H-
purine,
N-(2-(4-(9-benzyl-6-(1-methylcyclopropoxy)-9H-purin-8-yl)-3-chlorophenoxy)ethyl)-2-
hydroxyacetamide,
2-(4-(9-benzyl-6-(1-methylcyclopropoxy)-9H-purin-8-yl)-3-chlorophenoxy)acetamide,
2-(4-(9-benzyl-6-(1-methylcyclopropoxy)-9H-purin-8-yl)-3-chlorophenoxy)-N-(2-
hydroxyethyl)acetamide,
(2-(4-(9-benzyl-6-(1-methylcyclopropoxy)-9H-purin-8-yl)-3-chlorophenoxy)ethyl)glycine,
N-(2-(4-(9-benzyl-6-(1-methylcyclopropoxy)-9H-purin-8-yl)-3-chlorophenoxy)ethyl)-N-
methylglycine,
9-benzyl-8-(2-chloro-4-(((3S,4R)-4-fluoropyrrolidin-3-yl)oxy)phenyl)-6-(1-
methylcyclopropoxy)-9H-purine,
(R)-9-benzyl-8-(2-chloro-4-(piperidin-3-yloxy)phenyl)-6-(1-methylcyclopropoxy)-9H-purine,
9-benzyl-8-(2-chloro-4-(piperidin-4-yloxy)phenyl)-6-(1-methylcyclopropoxy)-9H-purine,
8-(4-((2-azaspiro[3.3]heptan-6-yl)oxy)-2-chlorophenyl)-9-benzyl-6-(1-methylcyclopropoxy)-
9H-purine,
8-(4-(azetidin-3-yloxy)-2-chlorophenyl)-9-benzyl-6-(1-methylcyclopropoxy)-9H-purine,
8-(2-chloro-4-(2-(4-methylpiperazin-1-yl)ethoxy)phenyl)-9-(2-chlorobenzyl)-6-(1-
methylcyclopropoxy)-9H-purine,
8-(2-chloro-4-(2-(4-methylpiperazin-1-yl)ethoxy)phenyl)-9-(3-chlorobenzyl)-6-(1-
methylcyclopropoxy)-9H-purine,
8-(2-chloro-4-(2-(4-methylpiperazin-1-yl)ethoxy)phenyl)-9-(2,3-difluorobenzyl)-6-(1-
methylcyclopropoxy)-9H-purine,
8-(2-chloro-4-(2-(4-methylpiperazin-1-yl)ethoxy)phenyl)-6-(1-methylcyclopropoxy)-9-
phenethyl-9H-purine,
8-(2-chloro-4-(2-(4-methylpiperazin-1-yl)ethoxy)phenyl)-6-(1-methylcyclopropoxy)-9-
(pyridin-2-ylmethyl)-9H-purine,
8-(2-chloro-4-(2-(4-methylpiperazin-1-yl)ethoxy)phenyl)-6-(1-methylcyclopropoxy)-9-
(pyrimidin-2-ylmethyl)-9H-purine,
8-(2-chloro-4-(2-(piperazin-1-yl)ethoxy)phenyl)-6-(1-methylcyclopropoxy)-9-(pyridin-2-
ylmethyl)-9H-purine,
1-(5-(9-benzyl-6-(1-methylcyclopropoxy)-9H-purin-8-yl)-4-methylpyridin-2-yl)piperidin-4-
amine,
1-(5-(9-benzyl-6-(1-methylcyclopropoxy)-9H-purin-8-yl)-4-methylpyridin-2-yl)-N,N-
dimethylazetidin-3-amine,
1-(5-(9-benzyl-6-(1-methylcyclopropoxy)-9H-purin-8-yl)-4-methylpyridin-2-yl)-N,N-
dimethylpiperidin-4-amine,
9-benzyl-8-(4-methyl-6-(3-(piperazin-1-yl)propoxy)pyridin-3-yl)-6-(1-methylcyclopropoxy)-
9H-purine,
3-((5-(9-benzyl-6-(1-methylcyclopropoxy)-9H-purin-8-yl)-4-methylpyridin-2-yl)oxy)-N,N-
dimethylpropan-1-amine,
1-(4-(3-((5-(9-benzyl-6-(1-methylcyclopropoxy)-9H-purin-8-yl)-4-methylpyridin-2-
yl)oxy)propyl)piperazin-1-yl)ethan-1-one,
1-(4-(2-((5-(9-benzyl-6-(1-methylcyclopropoxy)-9H-purin-8-yl)-4-methylpyridin-2-
yl)oxy)ethyl)piperazin-1-yl)ethan-1-one,
9-benzyl-8-(4-methyl-6-(3-(pyrrolidin-1-yl)propoxy)pyridin-3-yl)-6-(1-methylcyclopropoxy)-
9H-purine,
9-benzyl-8-(4-methyl-6-(2-(piperazin-1-yl)ethoxy)pyridin-3-yl)-6-(1-methylcyclopropoxy)-
9H-purine,
4-(3-((5-(9-benzyl-6-(1-methylcyclopropoxy)-9H-purin-8-yl)-4-methylpyridin-2-
yl)oxy)propyl)morpholine,
2-((5-(9-benzyl-6-(1-methylcyclopropoxy)-9H-purin-8-yl)-4-methylpyridin-2-yl)oxy)ethan-1-
amine,
3-((5-(9-benzyl-6-(1-methylcyclopropoxy)-9H-purin-8-yl)-4-methylpyridin-2-yl)oxy)propan-
1-amine,
1-(3-((5-(9-benzyl-6-(1-methylcyclopropoxy)-9H-purin-8-yl)-4-methylpyridin-2-
yl)oxy)azetidin-1-yl)ethan-1-one,
(S)-9-benzyl-8-(4-methyl-6-(pyrrolidin-3-yloxy)pyridin-3-yl)-6-(1-methylcyclopropoxy)-9H-
purine,
2-((5-(9-benzyl-6-(1-methylcyclopropoxy)-9H-purin-8-yl)-4-methylpyridin-2-yl)oxy)-N,N-
dimethylethan-1-amine,
(S)-9-benzyl-8-(4-methyl-6-(piperidin-3-yloxy)pyridin-3-yl)-6-(1-methylcyclopropoxy)-9H-
purine,
9-benzyl-8-(3-methyl-2-(2-(piperazin-1-yl)ethoxy)pyridin-4-yl)-6-(1-methylcyclopropoxy)-
9H-purine,
(R)-2-(((5-(9-benzyl-6-(1-methylcyclopropoxy)-9H-purin-8-yl)-4-methylpyridin-2-
yl)oxy)methyl)morpholine,
(R)-3-(((5-(9-benzyl-6-(1-methylcyclopropoxy)-9H-purin-8-yl)-4-methylpyridin-2-
yl)oxy)methyl)morpholine,
8-{6-[(azetidin-3-yl)oxy]-4-methylpyridin-3-yl}-9-benzyl-6-[(1-methylcyclopropyl)oxy]-9H-
purine,
9-benzyl-8-(2,6-difluoro-4-(2-(4-methylpiperazin-1-yl)ethoxy)phenyl)-6-(1-
methylcyclopropoxy)-9H-purine,
9-benzyl-8-(2-fluoro-4-(2-(4-methylpiperazin-1-yl)ethoxy)phenyl)-6-(1-methylcyclopropoxy)-
9H-purine,
9-benzyl-8-(2-methoxy-4-(2-(4-methylpiperazin-1-yl)ethoxy)phenyl)-6-(1-
methylcyclopropoxy)-9H-purine,
9-benzyl-8-(2,6-dimethyl-4-(2-(4-methylpiperazin-1-yl)ethoxy)phenyl)-6-(1-
methylcyclopropoxy)-9H-purine,
9-benzyl-8-(2,3-difluoro-4-(2-(4-methylpiperazin-1-yl)ethoxy)phenyl)-6-(1-
methylcyclopropoxy)-9H-purine,
9-benzyl-8-(2-bromo-4-(2-(4-methylpiperazin-1-yl)ethoxy)phenyl)-6-(1-
methylcyclopropoxy)-9H-purine,
8-(2-chloro-4-(2-(4-methylpiperazin-1-yl)ethoxy)phenyl)-6-(1-methylcyclopropoxy)-9-
(pyridin-3-ylmethyl)-9H-purine,
2-(9-benzyl-6-(1-methylcyclopropoxy)-9H-purin-8-yl)-5-(2-(4-methylpiperazin-1-
yl)ethoxy)benzonitrile,
9-benzyl-8-(2-ethynyl-4-(2-(4-methylpiperazin-1-yl)ethoxy)phenyl)-6-(1-
methylcyclopropoxy)-9H-purine,
(2-(9-benzyl-6-(1-methylcyclopropoxy)-9H-purin-8-yl)-5-(2-(4-methylpiperazin-1-
yl)ethoxy)phenyl)methanol,
3-(4-(9-benzyl-6-(1-methylcyclopropoxy)-9H-purin-8-yl)-3-chlorophenoxy)propanoic acid,
(2-(4-(9-benzyl-6-(1-methylcyclopropoxy)-9H-purin-8-yl)-3-chlorophenoxy)ethyl)-L-proline,
(R)-1-(2-(4-(9-benzyl-6-(1-methylcyclopropoxy)-9H-purin-8-yl)-3-
chlorophenoxy)ethyl)pyrrolidin-3-ol,
1-(2-(4-(9-benzyl-6-(1-methylcyclopropoxy)-9H-purin-8-yl)-3-chlorophenoxy)ethyl)azetidin-
3-ol,
1-(2-(4-(9-benzyl-6-(1-methylcyclopropoxy)-9H-purin-8-yl)-3-chlorophenoxy)ethyl)piperidin-
4-o1
2-(2-(4-(9-benzyl-6-(1-methylcyclopropoxy)-9H-purin-8-yl)-3-chlorophenoxy)ethyl)-2-
azaspiro[3.3]heptan-6-ol,
1-(3-(4-(9-benzyl-6-(1-methylcyclopropoxy)-9H-purin-8-yl)-3-
chlorophenoxy)propyl)azetidin-3-ol,
8-(4-(2-(1,6-diazaspiro[3.3]heptan-6-yl)ethoxy)-2-chlorophenyl)-9-benzyl-6-(1-
methylcyclopropoxy)-9H-purine,
(S)-4-(2-(4-(9-benzyl-6-(1-methylcyclopropoxy)-9H-purin-8-yl)-3-chlorophenoxy)ethyl)-3-
methylpiperazin-2-one,
6-(2-(4-(9-benzyl-6-(1-methylcyclopropoxy)-9H-purin-8-yl)-3-chlorophenoxy)ethyl)-2-thia-6-
azaspiro[3.3]heptane 2,2-dioxide,
1-(2-(4-(9-benzyl-6-(1-methylcyclopropoxy)-9H-purin-8-yl)-3-chlorophenoxy)ethyl)-1,4-
diazepan-5-one,
(R)-4-(2-(4-(9-benzyl-6-(1-methylcyclopropoxy)-9H-purin-8-yl)-3-chlorophenoxy)ethyl)-3-
methylpiperazin-2-one,
6-(2-(4-(9-benzyl-6-(1-methylcyclopropoxy)-9H-purin-8-yl)-3-chlorophenoxy)ethyl)-2-thia-6-
azaspiro[3.3]heptane,
9-benzyl-8-(2,3-dichloro-4-(2-(piperazin-1-yl)ethoxy)phenyl)-6-(1-methylcyclopropoxy)-9H-
purine,
9-benzyl-8-(2-chloro-5-methoxy-4-(2-(piperazin-1-yl)ethoxy)phenyl)-6-(1-
methylcyclopropoxy)-9H-purine,
9-benzyl-8-(2-chloro-3-methoxy-4-(2-(piperazin-1-yl)ethoxy)phenyl)-6-(1-
methylcyclopropoxy)-9H-purine,
9-benzyl-8-(2,5-dichloro-4-(2-(piperazin-1-yl)ethoxy)phenyl)-6-(1-methylcyclopropoxy)-9H-
purine,
9-(2-chlorobenzyl)-8-(4-methyl-6-(2-(piperazin-1-yl)ethoxy)pyridin-3-yl)-6-(1-
methylcyclopropoxy)-9H-purine,
9-(3-chlorobenzyl)-8-(4-methyl-6-(2-(piperazin-1-yl)ethoxy)pyridin-3-yl)-6-(1-
methylcyclopropoxy)-9H-purine,
8-(2-chloro-4-(2-(piperazin-1-yl)ethoxy)phenyl)-6-(1-methylcyclobutoxy)-9-(pyridin-2-
ylmethyl)-9H-purine,
8-(2-chloro-4-(2-(piperazin-1-yl)ethoxy)phenyl)-6-(1-methylcyclopropoxy)-9-(2-(pyridin-3-
yl)ethyl)-9H-purine,
(S)-8-(2-chloro-4-(2-(piperazin-1-yl)ethoxy)phenyl)-6-(1-methylcyclopropoxy)-9-(2-
phenylpropyl)-9H-purine,
(R)-8-(2-chloro-4-(2-(piperazin-1-yl)ethoxy)phenyl)-6-(1-methylcyclopropoxy)-9-(2-
phenylpropyl)-9H-purine,
8-(2-chloro-4-(2-(piperazin-1-yl)ethoxy)phenyl)-6-(1-methylcyclopropoxy)-9-(2-(pyridin-2-
yl)ethyl)-9H-purine,
8-(2-chloro-4-(2-(piperazin-1-yl)ethoxy)phenyl)-6-(1-methylcyclopropoxy)-9-phenethyl-9H-
purine,
8-(2-chloro-4-(2-(piperazin-1-yl)ethoxy)phenyl)-9-(3-chlorobenzyl)-6-(1-
methylcyclopropoxy)-9H-purine,
8-(2-chloro-4-(2-(4-methylpiperazin-1-yl)ethoxy)phenyl)-6-(1-methylcyclopropoxy)-9-
(pyridin-4-ylmethyl)-9H-purine,
4-((8-(2-chloro-4-(2-(piperazin-1-yl)ethoxy)phenyl)-6-(1-methylcyclopropoxy)-9H-purin-9-
yl)methyl)thiazole,
8-(2-chloro-4-(2-(piperazin-1-yl)ethoxy)phenyl)-9-((5-chloropyridin-2-yl)methyl)-6-(1-
methylcyclopropoxy)-9H-purine,
2-((8-(2-chloro-4-(2-(piperazin-1-yl)ethoxy)phenyl)-6-(1-methylcyclopropoxy)-9H-purin-9-
yl)methyl)thiazole,
8-(2-chloro-4-(2-(piperazin-1-yl)ethoxy)phenyl)-9-((3-chloropyridin-2-yl)methyl)-6-(1-
methylcyclopropoxy)-9H-purine,
4-((8-(2-chloro-4-(2-(piperazin-1-yl)ethoxy)phenyl)-6-(1-methylcyclopropoxy)-9H-purin-9-
yl)methyl)-2-methylthiazole,
8-(2-chloro-4-(2-(piperazin-1-yl)ethoxy)phenyl)-6-(1-methylcyclopropoxy)-9-((6-
methylpyridin-2-yl)methyl)-9H-purine,
8-(2-chloro-4-(2-(piperazin-1-yl)ethoxy)phenyl)-6-(1-methylcyclopropoxy)-9-((4-
(trifluoromethyl)pyridin-2-yl)methyl)-9H-purine,
6-((8-(2-chloro-4-(2-(piperazin-1-yl)ethoxy)phenyl)-6-(1-methylcyclopropoxy)-9H-purin-9-
yl)methyl)picolinonitrile,
8-(2-chloro-4-(2-(piperazin-1-yl)ethoxy)phenyl)-9-((6-methoxypyridin-2-yl)methyl)-6-(1-
methylcyclopropoxy)-9H-purine,
2-((8-(2-chloro-4-(2-(piperazin-1-yl)ethoxy)phenyl)-6-(1-methylcyclopropoxy)-9H-purin-9-
yl)methyl)nicotinonitrile,
4-((8-(2-chloro-4-(2-(piperazin-1-yl)ethoxy)phenyl)-6-(1-methylcyclopropoxy)-9H-purin-9-
yl)methyl)-5-methylthiazole,
8-(2-chloro-4-(2-(piperazin-1-yl)ethoxy)phenyl)-6-(1-methylcyclopropoxy)-9-(2-(pyridin-4-
yl)ethyl)-9H-purine,
8-(2-chloro-4-(2-(piperazin-1-yl)ethoxy)phenyl)-6-(1-methylcyclopropoxy)-9-((4-
methylpyridin-2-yl)methyl)-9H-purine,
2-((8-(2-chloro-4-(2-(piperazin-1-yl)ethoxy)phenyl)-6-(1-methylcyclopropoxy)-9H-purin-9-
yl)methyl)-5-methylthiazole,
2-((8-(2-chloro-4-(2-(piperazin-1-yl)ethoxy)phenyl)-6-(1-methylcyclopropoxy)-9H-purin-9-
yl)methyl)isonicotinonitrile,
8-(2-chloro-4-(2-(piperazin-1-yl)ethoxy)phenyl)-9-((4-methoxypyridin-2-yl)methyl)-6-(1-
methylcyclopropoxy)-9H-purine,
5-chloro-2-((8-(2-chloro-4-(2-(piperazin-1-yl)ethoxy)phenyl)-6-(1-methylcyclopropoxy)-9H-
purin-9-yl)methyl)thiazole,
2-chloro-4-((8-(2-chloro-4-(2-(piperazin-1-yl)ethoxy)phenyl)-6-(1-methylcyclopropoxy)-9H-
purin-9-yl)methyl)thiazole,
8-(2-chloro-4-(2-(piperazin-1-yl)ethoxy)phenyl)-6-(1-methylcyclopropoxy)-9-((5-
methylpyridin-2-yl)methyl)-9H-purine,
8-(2-chloro-4-(2-(piperazin-1-yl)ethoxy)phenyl)-6-(1-methylcyclopropoxy)-9-((3-
methylpyridin-2-yl)methyl)-9H-purine,
8-(2-chloro-4-(2-(piperazin-1-yl)ethoxy)phenyl)-9-((3-methoxypyridin-2-yl)methyl)-6-(1-
methylcyclopropoxy)-9H-purine,
1-(4-(9-benzyl-6-(1-methylcyclopropoxy)-9H-purin-8-yl)-3-chlorophenyl)azetidin-3-amine,
1-(4-(9-benzyl-6-(1-methylcyclopropoxy)-9H-purin-8-yl)-3-chlorophenyl)piperidin-4-amine,
4-(4-(9-benzyl-6-(1-methylcyclopropoxy)-9H-purin-8-yl)-3-chlorophenyl)piperazin-2-one,
(E)-3-(4-(9-benzyl-6-(1-methylcyclopropoxy)-9H-purin-8-yl)-3-chlorophenyl)acrylic acid,
1-(2-(4-(9-benzyl-6-(1-methylcyclopropoxy)-9H-purin-8-yl)-3-
chlorophenoxy)ethyl)piperazin-2-one,
4-(9-benzyl-6-(1-methylcyclopropoxy)-9H-purin-8-yl)-3-chloro-N,N-dimethylbenzamide,
4-(9-benzyl-6-(1-methylcyclopropoxy)-9H-purin-8-yl)-3-chloro-N-methylbenzamide,
(4-(9-benzyl-6-(1-methylcyclopropoxy)-9H-purin-8-yl)-3-chlorophenyl)(piperazin-1-
yl)methanone,
4-(9-benzyl-6-(1-methylcyclopropoxy)-9H-purin-8-yl)-3-chlorobenzamide,
9-benzyl-8-(2-chloro-4-(pyrrolidin-1-ylmethyl)phenyl)-6-(1-methylcyclopropoxy)-9H-purine,
9-benzyl-8-(2-chloro-4-(piperazin-1-ylmethyl)phenyl)-6-(1-methylcyclopropoxy)-9H-purine,
(4-(9-benzyl-6-(1-methylcyclopropoxy)-9H-purin-8-yl)-3-chlorophenyl)methanamine,
9-benzyl-8-(2-chloro-4-methoxyphenyl)-6-(1-methylcyclopropoxy)-9H-purine,
9-benzyl-8-(2-methoxypyridin-3-yl)-6-(1-methylcyclopropoxy)-9H-purine,
2-(2-chloro-4-(2-(piperazin-1-yl)ethoxy)phenyl)-4-(1-methylcyclobutoxy)-1-(pyridin-2-
ylmethyl)-1H-benzo[d]imidazole,
1-benzyl-2-(2-chloro-4-(2-(piperazin-1-yl)ethoxy)phenyl)-4-(1-methylcyclobutoxy)-1H-
benzo[d]imidazole,
2-(2-chloro-4-(2-(piperazin-1-yl)ethoxy)phenyl)-7-(1-methylcyclobutoxy)-3-(pyridin-2-
ylmethyl)-3H-imidazo[4,5-b]pyridine,
3-benzyl-2-(2-chloro-4-(2-(piperazin-1-yl)ethoxy)phenyl)-7-(1-methylcyclobutoxy)-3H-
imidazo[4,5-b]pyridine,
9-benzyl-8-(2-chloro-4-(2-(piperazin-1-yl)ethoxy)phenyl)-6-(1-ethynylcyclopropoxy)-9H-
purine,
9-benzyl-8-(2-chloro-4-(2-(piperazin-1-yl)ethoxy)phenyl)-6-(1-ethylcyclopropoxy)-9H-purine,
9-benzyl-8-(2-chloro-4-(2-(piperazin-1-yl)ethoxy)phenyl)-6-(1-ethynylcyclobutoxy)-9H-
purine,
8-(2-chloro-4-(2-(piperazin-1-yl)ethoxy)phenyl)-9-(3-chlorobenzyl)-6-cyclopropoxy-9H-
purine,
1-((8-(2-chloro-4-(2-(piperazin-1-yl)ethoxy)phenyl)-9-(3-chlorobenzyl)-9H-purin-6-
yl)oxy)cyclopropane-1-carbonitrile,
2-(2-(3-chloro-4-(9-((4-chloropyridin-2-yl)methyl)-6-(1-methylcyclopropoxy)-9H-purin-8-
yl)phenoxy)ethyl)-2-azaspiro[3.3]heptan-6-ol,
(R)-1-(2-(3-chloro-4-(9-((4-chloropyridin-2-yl)methyl)-6-(1-methylcyclopropoxy)-9H-purin-
8-yl)phenoxy)ethyl)pyrrolidin-3-ol,
(S)-9-benzyl-8-(2-chloro-4-(2-(3-methylpiperazin-1-yl)ethoxy)phenyl)-6-(1-
methylcyclopropoxy)-9H-purine,
8-(4-(2-(3,8-diazabicyclo[3.2.1]octan-3-yl)ethoxy)-2-chlorophenyl)-9-benzyl-6-(1-
methylcyclopropoxy)-9H-purine,
3-(2-(4-(9-benzyl-6-(1-methylcyclopropoxy)-9H-purin-8-yl)-3-chlorophenoxy)ethyl)-3,6-
diazabicyclo[3.1.1 ]heptane,
(R)-9-benzyl-8-(2-chloro-4-(2-(3-methylpiperazin-1-yl)ethoxy)phenyl)-6-(1-
methylcyclopropoxy)-9H-purine,
(R)-1-(2-(4-(9-benzyl-6-(1-methylcyclopropoxy)-9H-purin-8-yl)-3-
chlorophenoxy)ethyl)piperidin-3-ol,
2-(2-(4-(9-benzyl-6-(1-methylcyclopropoxy)-9H-purin-8-yl)-3-chlorophenoxy)ethyl)-2,6-
diazaspiro[3.4]octan-7-one,
(cis)-9-benzyl-8-(2-chloro-4-(2-(3,5-dimethylpiperazin-1-yl)ethoxy)phenyl)-6-(1-
methylcyclopropoxy)-9H-purine,
(S)-9-benzyl-8-(2-chloro-4-(2-(2-methylpiperazin-1-yl)ethoxy)phenyl)-6-(1-
methylcyclopropoxy)-9H-purine,
(R)-9-benzyl-8-(2-chloro-4-(2-(2-methylpiperazin-1-yl)ethoxy)phenyl)-6-(1-
methylcyclopropoxy)-9H-purine,
1-(2-(4-(9-benzyl-6-(1-methylcyclopropoxy)-9H-purin-8-yl)-3-chlorophenoxy)ethyl)-1,4-
diazepan-2-one,
1-(2-(4-(9-benzyl-6-(1-methylcyclopropoxy)-9H-purin-8-yl)-3-chlorophenoxy)ethyl)-3-
methylpiperazin-2-one (Isomer 1),
1-(2-(4-(9-benzyl-6-(1-methylcyclopropoxy)-9H-purin-8-yl)-3-chlorophenoxy)ethyl)-3-
methylpiperazin-2-one (Isomer 2),
6-(2-(4-(9-benzyl-6-(1-methylcyclopropoxy)-9H-purin-8-yl)-3-chlorophenoxy)ethyl)-3,6-
diazabicyclo[3.1.1]heptane,
1-(2-(4-(9-benzyl-6-(1-methylcyclopropoxy)-9H-purin-8-yl)-3-chlorophenoxy)ethyl)-3,3-
dimethylpiperazin-2-one,
8-(4-(2-(4,7-diazaspiro[2.5]octan-7-yl)ethoxy)-2-chlorophenyl)-9-benzyl-6-(1-
methylcyclopropoxy)-9H-purine,
(R)-(4-(2-(4-(9-benzyl-6-(1-methylcyclopropoxy)-9H-purin-8-yl)-3-
chlorophenoxy)ethyl)piperazin-2-yl)methanol,
8-(4-(2-((1S,4S)-2,5-diazabicyclo[2.2.1]heptan-2-yl)ethoxy)-2-chlorophenyl)-9-benzyl-6-(1-
methylcyclopropoxy)-9H-purine,
8-(4-(2-((1R,4R)-2,5-diazabicyclo[2.2.1]heptan-2-yl)ethoxy)-2-chlorophenyl)-9-benzyl-6-(1-
methylcyclopropoxy)-9H-purine,
(1S,4S)-2-(2-(4-(9-benzyl-6-(1-methylcyclopropoxy)-9H-purin-8-yl)-3-chlorophenoxy)ethyl)-
2,5-diazabicyclo[2.2.2]octane,
8-(4-(2-(4,7-diazaspiro[2.5]octan-4-yl)ethoxy)-2-chlorophenyl)-9-benzyl-6-(1-
methylcyclopropoxy)-9H-purine,
(S)-2-(4-(2-(4-(9-benzyl-6-(1-methylcyclopropoxy)-9H-purin-8-yl)-3-
chlorophenoxy)ethyl)piperazin-2-yl)acetonitrile,
1-(3-(4-(9-benzyl-6-(1-methylcyclopropoxy)-9H-purin-8-yl)-3-
chlorophenoxy)propyl)piperazin-2-one,
4-(3-(4-(9-benzyl-6-(1-methylcyclopropoxy)-9H-purin-8-yl)-3-
chlorophenoxy)propyl)piperazin-2-one,
8-(2-chloro-4-(2-(piperazin-1-yl)ethoxy)phenyl)-9-((4-cyclopropylpyridin-2-yl)methyl)-6-(1-
methylcyclopropoxy)-9H-purine,
8-(2-chloro-4-(2-(piperazin-1-yl)ethoxy)phenyl)-9-((4-ethylpyridin-2-yl)methyl)-6-(1-
methylcyclopropoxy)-9H-purine,
8-(2-chloro-4-(2-(piperazin-1-yl)ethoxy)phenyl)-9-(1-(4-chloropyridin-2-yl)ethyl)-6-(1-
methylcyclopropoxy)-9H-purine (Isomer 2),
8-(2-chloro-4-(2-(piperazin-1-yl)ethoxy)phenyl)-9-(1-(4-chloropyridin-2-yl)ethyl)-6-(1-
methylcyclopropoxy)-9H-purine (Isomer 1),
(R)-8-(2-chloro-4-(2-(piperazin-1-yl)ethoxy)phenyl)-9-(1-(4-chloropyridin-2-yl)ethyl)-6-(1-
methylcyclopropoxy)-9H-purine,
(S)-8-(2-chloro-4-(2-(piperazin-1-yl)ethoxy)phenyl)-9-(1-(4-chloropyridin-2-yl)ethyl)-6-(1-
methylcyclopropoxy)-9H-purine,
8-(2-chloro-4-(2-(piperazin-1-yl)ethoxy)phenyl)-6-(1-methylcyclopropoxy)-9-(1-(pyridin-2-
yl)propan-2-yl)-9H-purine (Isomer 2),
8-(2-chloro-4-(2-(piperazin-1-yl)ethoxy)phenyl)-6-(1-methylcyclopropoxy)-9-(1-(pyridin-2-
yl)propan-2-yl)-9H-purine (Isomer 1),
(R)-8-(2-chloro-4-(2-(piperazin-1-yl)ethoxy)phenyl)-6-(1-methylcyclopropoxy)-9-(1-(pyridin-
2-yl)propan-2-yl)-9H-purine,
(S)-8-(2-chloro-4-(2-(piperazin-1-yl)ethoxy)phenyl)-6-(1-methylcyclopropoxy)-9-(1-(pyridin-
2-yl)propan-2-yl)-9H-purine,

TABLE II
2-(3-Chloro-4-(6-(1-methylcyclopropoxy)-9-((4-methylpyridin-2-yl)methyl)-9H-purin-8-
yl)phenyl)acetamide
E-3-(3-Chloro-4-(9-(3-chlorobenzyl)-6-(1-methylcyclopropoxy)-9H-purin-8-yl)phenyl)acrylic
acid
3-(3-Chloro-4-(9-(3-chlorobenzyl)-6-(1-methylcyclopropoxy)-9H-purin-8-yl)phenoxy)-22-
dimethylpropanoic acid
2-(3-Chloro-4-(6-(1-methylcyclopropoxy)-9-((4-methylpyridin-2-yl)methyl)-9H-purin-8-
yl)phenoxy)ethan-1-ol
4-(3-Chloro-4-(9-(3-chlorobenzyl)-6-(1-methylcyclopropoxy)-9H-purin-8-yl)phenoxy)-2-
methylbutanoic acid
4-(3-Chloro-4-(9-(3-chlorobenzyl)-6-(1-methylcyclopropoxy)-9H-purin-8-yl)phenoxy)-2-
methylbutanoic acid (Isomer 1)
4-(3-Chloro-4-(9-(3-chlorobenzyl)-6-(1-methylcyclopropoxy)-9H-purin-8-yl)phenoxy)-2-
methylbutanoic acid (Isomer 2)
(R)-4-(3-Chloro-4-(9-(3-chlorobenzyl)-6-(1-methylcyclopropoxy)-9H-purin-8-yl)phenoxy)-2-
methylbutanoic acid
(S)-4-(3-Chloro-4-(9-(3-chlorobenzyl)-6-(1-methylcyclopropoxy)-9H-purin-8-yl)phenoxy)-2-
methylbutanoic acid
(S)-1-(3-Chloro-4-(6-(1-methylcyclopropoxy)-9-((4-methylpyridin-2-yl)methyl)-9H-purin-8-
yl)phenoxy)propan-2-ol
(R)-1-(3-Chloro-4-(6-(1-methylcyclopropoxy)-9-((4-methylpyridin-2-yl)methyl)-9H-purin-8-
yl)phenoxy)propan-2-ol
1-(3-Chloro-4-(6-(1-methylcyclopropoxy)-9-((4-methylpyridin-2-yl)methyl)-9H-purin-8-
yl)phenoxy)propan-2-ol
2-(3-Chloro-4-(9-((4-chloropyridin-2-yl)methyl)-6-(1-methylcyclopropoxy)-9H-purin-8-
yl)phenyl)acetamide
1-(3-(3-Chloro-4-(6-(1-methylcyclopropoxy)-9-((4-methylpyridin-2-yl)methyl)-9H-purin-8-
yl)phenoxy)100zetidine-1-yl)-2-hydroxyethan-1-one
1-(3-(3-Chloro-4-(6-(1-methylcyclopropoxy)-9-((4-methylpyridin-2-yl)methyl)-9H-purin-8-
yl)phenoxy)100zetidine-1-yl)ethan-1-one
(S)-5-(3-Chloro-4-(6-(1-methylcyclopropoxy)-9-((4-methylpyridin-2-yl)methyl)-9H-purin-8-
yl)phenoxy)piperidin-2-one
5-(3-Chloro-4-(6-(1-methylcyclopropoxy)-9-((4-methylpyridin-2-yl)methyl)-9H-purin-8-
yl)phenoxy)tetrahydropyrimidin-2(1H)-one
5-(3-Chloro-4-(9-(3-chlorobenzyl)-6-(1-methylcyclopropoxy)-9H-purin-8-
yl)phenoxy)pentanoic acid
4-(3-Chloro-4-(9-(3-chlorobenzyl)-6-(1-methylcyclopropoxy)-9H-purin-8-yl)phenoxy)butanoic
acid
1-(3-Chloro-4-(6-(1-methylcyclopropoxy)-9-((4-methylpyridin-2-yl)methyl)-9H-purin-8-
yl)phenoxy)-2-methylpropan-2-ol
2-(2-(3-Chloro-4-(9-(3-chlorobenzyl)-6-(1-methylcyclopropoxy)-9H-purin-8-
yl)phenoxy)ethoxy)acetic acid
(S)-4-(2-Chloro-3-(9-(5-chloro-2-cyanobenzyl)-6-(1-methylcyclopropoxy)-9H-purin-8-
yl)phenoxy)-2-methylbutanoic acid

TABLE III
(R)-4-(3-chloro-4-(9-(5-chloro-2-methoxybenzyl)-6-(1-methylcyclopropoxy)-9H-purin-8-
yl)phenoxy)-2-methylbutanoic acid
(S)-4-(3-chloro-4-(9-(5-chloro-2-methoxybenzyl)-6-(1-methylcyclopropoxy)-9H-purin-8-
yl)phenoxy)-2-methylbutanoic acid
3-(2-Chloro-3-(9-(5-chloro-2-methoxybenzyl)-6-(1-methylcyclopropoxy)-9H-purin-8-
yl)phenoxy)propanoic acid
(R)-4-(2-chloro-3-(9-(5-chloro-2-methoxybenzyl)-6-(1-methylcyclopropoxy)-9H-purin-8-
yl)phenoxy)-3-methylbutanoic acid
(S)-4-(2-chloro-3-(9-(5-chloro-2-methoxybenzyl)-6-(1-methylcyclopropoxy)-9H-purin-8-
yl)phenoxy)-3-methylbutanoic acid
(1s,3s)-3-(2-chloro-3-(9-(5-chloro-2-methoxybenzyl)-6-(1-methylcyclopropoxy)-9H-purin-8-
yl)phenoxy)cyclobutane-1-carboxylic acid
(R)-4-(2-Chloro-3-(9-(5-chloro-2-cyanobenzyl)-6-(1-methylcyclopropoxy)-9H-purin-8-
yl)phenoxy)-2-methylbutanoic acid
(S)-4-(2-Chloro-3-(9-(5-chloro-2-cyanobenzyl)-6-(1-methylcyclopropoxy)-9H-purin-8-
yl)phenoxy)-2-methylbutanoic acid
2-(3-chloro-4-(9-((5-methyl-1,3,4-thiadiazol-2-yl)methyl)-6-(1-methylcyclopropoxy)-9H-purin-
8-yl)phenyl)acetamide
2-(3-chloro-4-(9-((5-methyl-1,3,4-oxadiazol-2-yl)methyl)-6-(1-methylcyclopropoxy)-9H-purin-
8-yl)phenyl)acetamide
3-chloro-4-(6-(1-methylcyclopropoxy)-9-((4-methylpyridin-2-yl)methyl)-9H-purin-8-yl)aniline
2-(3-chloro-4-(6-(1-methylcyclopropoxy)-9-((5-methylthiazol-2-yl)methyl)-9H-purin-8-
yl)phenyl)acetamide
2-(3-chloro-4-(6-(1-methylcyclopropoxy)-9-((2-methylthiazol-5-yl)methyl)-9H-purin-8-
yl)phenyl)acetamide
2-(3-chloro-4-(6-(1-methylcyclopropoxy)-9-((2-methylthiazol-4-yl)methyl)-9H-purin-8-
yl)phenyl)acetamide
2-(3-chloro-4-(6-(1-methylcyclopropoxy)-9-(2-(thiazol-2-yl)ethyl)-9H-purin-8-
yl)phenyl)acetamide
2-(3-chloro-4-(6-(1-methylcyclopropoxy)-9-(thiazol-5-ylmethyl)-9H-purin-8-yl)phenoxy)-N-
methylacetamide
3-chloro-4-(6-(1-methylcyclopropoxy)-9-((4-methylpyridin-2-yl)methyl)-9H-purin-8-
yl)benzenesulfonamide
2-(3-chloro-4-(9-((5-ethyl-1,3,4-oxadiazol-2-yl)methyl)-6-(1-methylcyclopropoxy)-9H-purin-8-
yl)phenyl)acetamide
3-chloro-4-(9-(3-chlorobenzyl)-6-(1-methylcyclopropoxy)-9H-purin-8-yl)benzoic acid
2-chloro-3-(9-(3-chlorobenzyl)-6-(1-methylcyclopropoxy)-9H-purin-8-yl)benzoic acid
3-chloro-4-(6-(1-methylcyclopropoxy)-9-((4-methylpyridin-2-yl)methyl)-9H-purin-8-yl)benzoic
acid
3-chloro-4-(6-(1-methylcyclopropoxy)-9-((4-methylpyridin-2-yl)methyl)-9H-purin-8-
yl)benzamide
2-(3-chloro-4-(9-((4-chloropyridin-2-yl)methyl)-6-(1-methylcyclopropoxy)-9H-purin-8-
yl)phenoxy)ethan-1-ol
8-(2-chloro-4-(methylsulfonyl)phenyl)-6-(1-methylcyclopropoxy)-9-((4-methylpyridin-2-
yl)methyl)-9H-purine
2-(3-chloro-4-(6-(1-methylcyclopropoxy)-9-((3-methylpyridazin-4-yl)methyl)-9H-purin-8-
yl)phenyl)acetamide
2-(3-chloro-4-(6-(1-methylcyclopropoxy)-9-((5-methylpyridazin-3-yl)methyl)-9H-purin-8-
yl)phenyl)acetamide
2-(3-chloro-4-(6-(1-methylcyclopropoxy)-9-((6-methylpyrimidin-4-yl)methyl)-9H-purin-8-
yl)phenyl)acetamide
2-(3-chloro-4-(9-((5-cyclopropyl-1,3,4-thiadiazol-2-yl)methyl)-6-(1-methylcyclopropoxy)-9H-
purin-8-yl)phenyl)acetamide
2-(3-chloro-4-(9-((5-cyclopropyl-1,3,4-oxadiazol-2-yl)methyl)-6-(1-methylcyclopropoxy)-9H-
purin-8-yl)phenyl)acetamide
2-(3-chloro-4-(9-((5-methoxypyridazin-3-yl)methyl)-6-(1-methylcyclopropoxy)-9H-purin-8-
yl)phenyl)acetamide
2-(3-chloro-4-(9-((3-cyclopropyl-1,2,4-oxadiazol-5-yl)methyl)-6-(1-methylcyclopropoxy)-9H-
purin-8-yl)phenyl)acetamide
2-(3-chloro-4-(9-((4,5-dimethylthiazol-2-yl)methyl)-6-(1-methylcyclopropoxy)-9H-purin-8-
yl)phenyl)acetamide
2-(3-chloro-4-(9-((2,4-dimethylthiazol-5-yl)methyl)-6-(1-methylcyclopropoxy)-9H-purin-8-
yl)phenyl)acetamide
2-(3-chloro-4-(6-(1-methylcyclopropoxy)-9-(2-(4-methylthiazol-5-yl)ethyl)-9H-purin-8-
yl)phenyl)acetamide
2-(3-chloro-4-(9-((3,5-dimethylisoxazol-4-yl)methyl)-6-(1-methylcyclopropoxy)-9H-purin-8-
yl)phenyl)acetamide
2-(3-chloro-4-(6-(1-methylcyclopropoxy)-9-((5-methylthiazol-2-yl)methyl)-9H-purin-8-
yl)phenoxy)-N-methylacetamide
N-(3-chloro-4-(6-(1-methylcyclopropoxy)-9-((4-methylpyridin-2-yl)methyl)-9H-purin-8-
yl)phenyl)methanesulfonamide
3-chloro-N-methyl-4-(6-(1-methylcyclopropoxy)-9-((4-methylpyridin-2-yl)methyl)-9H-purin-8-
yl)benzenesulfonamide
2-(3-chloro-4-(9-((1,4-dimethyl-1H-pyrazol-5-yl)methyl)-6-(1-methylcyclopropoxy)-9H-purin-
8-yl)phenyl)acetamide
2-(3-chloro-4-(9-((1,3-dimethyl-1H-pyrazol-5-yl)methyl)-6-(1-methylcyclopropoxy)-9H-purin-
8-yl)phenyl)acetamide
2-(3-chloro-4-(9-((1,3-dimethyl-1H-pyrazol-4-yl)methyl)-6-(1-methylcyclopropoxy)-9H-purin-
8-yl)phenyl)acetamide
2-(3-chloro-4-(6-(1-methylcyclopropoxy)-9-((5-methylthiazol-2-yl)methyl)-9H-purin-8-
yl)phenoxy)-N-methylethan-1-amine
2-(3-chloro-4-(6-(1-methylcyclopropoxy)-9-((2-(trifluoromethyl)pyridin-4-yl)methyl)-9H-
purin-8-yl)phenyl)acetamide
2-(3-chloro-4-(6-(1-methylcyclopropoxy)-9-((4-(trifluoromethyl)pyridin-2-yl)methyl)-9H-
purin-8-yl)phenyl)acetamide
2-(3-chloro-4-(6-(1-methylcyclopropoxy)-9-((2-(trifluoromethyl)pyridin-3-yl)methyl)-9H-
purin-8-yl)phenyl)acetamide
2-(3-chloro-4-(6-(1-methylcyclopropoxy)-9-((6-(trifluoromethyl)pyridin-3-yl)methyl)-9H-
purin-8-yl)phenyl)acetamide
2-(3-chloro-4-(9-((5-cyanopyridin-3-yl)methyl)-6-(1-methylcyclopropoxy)-9H-purin-8-
yl)phenyl)acetamide
2-(3-chloro-4-(9-((2-cyanopyridin-4-yl)methyl)-6-(1-methylcyclopropoxy)-9H-purin-8-
yl)phenyl)acetamide
2-(2-chloro-3-(9-(3-chlorobenzyl)-6-(1-methylcyclopropoxy)-9H-purin-8-yl)phenoxy)acetic
acid
2-(3-chloro-4-(9-(3-fluorobenzyl)-6-(1-methylcyclopropoxy)-9H-purin-8-yl)phenyl)acetamide
2-(3-chloro-4-(9-(4-fluorobenzyl)-6-(1-methylcyclopropoxy)-9H-purin-8-yl)phenyl)acetamide
2-(3-chloro-4-(6-(1-methylcyclopropoxy)-9-((4-(trifluoromethyl)pyridin-2-yl)methyl)-9H-
purin-8-yl)phenoxy)ethan-1-ol
2-(3-chloro-4-(9-(3-chlorobenzyl)-6-(1-methylcyclopropoxy)-9H-purin-8-yl)phenyl)acetamide
2-(4-(9-(2-(6-bromopyridin-2-yl)ethyl)-6-(1-methylcyclopropoxy)-9H-purin-8-yl)-3-
chlorophenyl)acetamide
8-(2-chloro-4-(2,2-difluoroethoxy)phenyl)-6-(1-methylcyclopropoxy)-9-((4-methylpyridin-2-
yl)methyl)-9H-purine
2-(4-(9-benzyl-6-(1-methylcyclopropoxy)-9H-purin-8-yl)-3-chlorophenyl)acetamide
2-(3-chloro-4-(6-(1-methylcyclopropoxy)-9-((4-methylpyridin-2-yl)methyl)-9H-purin-8-
yl)phenyl)acetic acid
8-(4-(azetidin-3-yloxy)-2-chlorophenyl)-9-((4-chloropyridin-2-yl)methyl)-6-(1-
methylcyclopropoxy)-9H-purine
2-(3-chloro-4-(9-((4-chloropyridin-2-yl)methyl)-6-(1-methylcyclopropoxy)-9H-purin-8-
yl)phenoxy)-N-methylacetamide
3-chloro-N-methyl-4-(6-(1-methylcyclopropoxy)-9-((4-methylpyridin-2-yl)methyl)-9H-purin-8-
yl)benzamide
N-(3-chloro-4-(6-(1-methylcyclopropoxy)-9-((4-methylpyridin-2-yl)methyl)-9H-purin-8-
yl)phenyl)acetamide
4-chloro-N-methyl-3-(6-(1-methylcyclopropoxy)-9-((4-methylpyridin-2-yl)methyl)-9H-purin-8-
yl)benzamide
2-chloro-N-methyl-3-(6-(1-methylcyclopropoxy)-9-((4-methylpyridin-2-yl)methyl)-9H-purin-8-
yl)benzamide
2-(2-chloro-3-(6-(1-methylcyclopropoxy)-9-((4-methylpyridin-2-yl)methyl)-9H-purin-8-
yl)phenyl)acetamide
2-(4-chloro-3-(6-(1-methylcyclopropoxy)-9-((4-methylpyridin-2-yl)methyl)-9H-purin-8-
yl)phenyl)acetamide
2-(3-chloro-4-(6-(1-methylcyclopropoxy)-9-((2-methylpyridin-4-yl)methyl)-9H-purin-8-
yl)phenyl)acetamide
2-(3-chloro-4-(6-(1-methylcyclopropoxy)-9-((6-methylpyridin-2-yl)methyl)-9H-purin-8-
yl)phenyl)acetamide
2-(3-chloro-4-(6-(1-methylcyclopropoxy)-9-((2-methylpyridin-3-yl)methyl)-9H-purin-8-
yl)phenyl)acetamide
2-(3-chloro-4-(6-(1-methylcyclopropoxy)-9-((4-methylpyridin-2-yl)methyl)-9H-purin-8-
yl)phenoxy)acetamide
methyl (3-chloro-4-(6-(1-methylcyclopropoxy)-9-((4-methylpyridin-2-yl)methyl)-9H-purin-8-
yl)phenyl)carbamate
2-(3-chloro-4-(9-((4-methoxypyridin-2-yl)methyl)-6-(1-methylcyclopropoxy)-9H-purin-8-
yl)phenyl)acetamide
3-(3-chloro-4-(9-((4-chloropyridin-2-yl)methyl)-6-(1-methylcyclopropoxy)-9H-purin-8-
yl)phenoxy)propan-1-ol
2-(3-chloro-4-(6-(1-methylcyclopropoxy)-9-((4-methylpyridin-2-yl)methyl)-9H-purin-8-
yl)phenyl)ethan-1-ol
1-(3-chloro-4-(6-(1-methylcyclopropoxy)-9-((4-methylpyridin-2-yl)methyl)-9H-purin-8-
yl)phenyl)-3-methylurea
2-(3-chloro-4-(9-((4-chloropyridin-2-yl)methyl)-6-(1-methylcyclopropoxy)-9H-purin-8-
yl)phenoxy)-N-methylethan-1-amine
1-(3-chloro-4-(6-(1-methylcyclopropoxy)-9-((4-methylpyridin-2-yl)methyl)-9H-purin-8-
yl)phenyl)-N-methylmethanamine
2-(3-chloro-4-(6-(1-methylcyclopropoxy)-9-((4-methylpyridin-2-yl)methyl)-9H-purin-8-
yl)phenoxy)ethan-1-amine
(S)-2-((8-(2-chloro-4-(pyrrolidin-3-yloxy)phenyl)-6-(1-methylcyclopropoxy)-9H-purin-9-
yl)methyl)-5-methylthiazole
2-(4-(9-((5-(tert-butyl)-1,2,4-oxadiazol-3-yl)methyl)-6-(1-methylcyclopropoxy)-9H-purin-8-yl)-
3-chlorophenyl)acetamide
2-(3-chloro-4-(6-(1-methylcyclopropoxy)-9-((5-methylthiazol-2-yl)methyl)-9H-purin-8-
yl)phenoxy)-N,N-dimethylethan-1-amine
2-(2-chloro-3-(6-(1-methylcyclopropoxy)-9-((5-methylthiazol-2-yl)methyl)-9H-purin-8-
yl)phenoxy)-N,N-dimethylethan-1-amine
3-(3-chloro-4-(6-(1-methylcyclopropoxy)-9-((5-methylthiazol-2-yl)methyl)-9H-purin-8-
yl)phenoxy)-N-methylpropan-1-amine
2-((8-(2-chloro-4-(2-(piperazin-1-yl)ethoxy)phenyl)-6-(1-methylcyclopropoxy)-9H-purin-9-
yl)methyl)-1,3,4-thiadiazole
2-((8-(2-chloro-4-(2-(piperazin-1-yl)ethoxy)phenyl)-6-(1-methylcyclopropoxy)-9H-purin-9-
yl)methyl)-1,3,4-oxadiazole
2-(3-chloro-4-(9-(5-cyano-2-fluorobenzyl)-6-(1-methylcyclopropoxy)-9H-purin-8-
yl)phenyl)acetamide
2-(3-chloro-4-(9-(2-cyano-4-fluorobenzyl)-6-(1-methylcyclopropoxy)-9H-purin-8-
yl)phenyl)acetamide
2-(3-chloro-4-(9-(2-cyano-5-fluorobenzyl)-6-(1-methylcyclopropoxy)-9H-purin-8-
yl)phenyl)acetamide
(E)-3-(2-chloro-3-(9-(3-chlorobenzyl)-6-(1-methylcyclopropoxy)-9H-purin-8-yl)phenyl)acrylic
acid
2-(3-chloro-4-(9-(3-cyanobenzyl)-6-(1-methylcyclopropoxy)-9H-purin-8-yl)phenyl)acetamide
2-(3-chloro-4-(9-(2-cyanobenzyl)-6-(1-methylcyclopropoxy)-9H-purin-8-yl)phenyl)acetamide
2-(3-chloro-4-(9-(4-cyanobenzyl)-6-(1-methylcyclopropoxy)-9H-purin-8-yl)phenyl)acetamide
3-(3-chloro-4-(9-(3,4-dichlorobenzyl)-6-(1-methylcyclopropoxy)-9H-purin-8-
yl)phenoxy)propanoic acid
3-(3-chloro-4-(9-(2,3-dichlorobenzyl)-6-(1-methylcyclopropoxy)-9H-purin-8-
yl)phenoxy)propanoic acid
3-chloro-4-(6-(1-methylcyclopropoxy)-9-((4-methylpyridin-2-yl)methyl)-9H-purin-8-yl)-N-
(2,2,2-trifluoroethyl)benzamide
3-(3-chloro-4-(9-(3-chlorobenzyl)-6-(1-methylcyclopropoxy)-9H-purin-8-yl)phenoxy)propanoic
acid
3-(3-chloro-4-(9-(2-chlorobenzyl)-6-(1-methylcyclopropoxy)-9H-purin-8-yl)phenoxy)propanoic
acid
3-(2-chloro-3-(9-(3-chlorobenzyl)-6-(1-methylcyclopropoxy)-9H-purin-8-yl)phenoxy)propanoic
acid
(S)-2-(2-chloro-3-(9-(3-chlorobenzyl)-6-(1-methylcyclopropoxy)-9H-purin-8-
yl)phenoxy)propanoic acid
(R)-2-(2-chloro-3-(9-(3-chlorobenzyl)-6-(1-methylcyclopropoxy)-9H-purin-8-
yl)phenoxy)propanoic acid
8-(4-(2-(2H-tetrazol-5-yl)ethoxy)-2-chlorophenyl)-9-(3-chlorobenzyl)-6-(1-
methylcyclopropoxy)-9H-purine
3-chloro-N-(2,2-difluoroethyl)-4-(6-(1-methylcyclopropoxy)-9-((4-methylpyridin-2-yl)methyl)-
9H-purin-8-yl)benzamide
3-(3-chloro-4-(6-(1-methylcyclopropoxy)-9-((4-(trifluoromethyl)pyridin-2-yl)methyl)-9H-
purin-8-yl)phenoxy)propan-1-ol
1-(3-chloro-4-(6-(1-methylcyclopropoxy)-9-((4-methylpyridin-2-yl)methyl)-9H-purin-8-
yl)phenyl)azetidin-2-one
3-(3-chloro-4-(6-(1-methylcyclopropoxy)-9-((4-methylpyridin-2-yl)methyl)-9H-purin-8-
yl)phenyl)oxazolidin-2-one
3-((3-chloro-4-(9-(3-chlorobenzyl)-6-(1-methylcyclopropoxy)-9H-purin-8-
yl)phenyl)amino)propanoic acid
3-(3-chloro-4-(6-(1-methylcyclopropoxy)-9-((4-methylpyridin-2-yl)methyl)-9H-purin-8-
yl)phenoxy)-2,2-difluoropropan-1-ol
2-(3-chloro-4-(9-(3-methylbenzyl)-6-(1-methylcyclopropoxy)-9H-purin-8-yl)phenyl)acetamide
2-(3-chloro-4-(9-(4-methylbenzyl)-6-(1-methylcyclopropoxy)-9H-purin-8-yl)phenyl)acetamide
2-(3-chloro-4-(9-(2-methylbenzyl)-6-(1-methylcyclopropoxy)-9H-purin-8-yl)phenyl)acetamide
2-(3-chloro-4-(6-(1-methylcyclopropoxy)-9-phenethyl-9H-purin-8-yl)phenyl)acetamide
8-(2-chloro-4-(oxetan-3-yloxy)phenyl)-6-(1-methylcyclopropoxy)-9-((4-methylpyridin-2-
yl)methyl)-9H-purine
1-(3-chloro-4-(6-(1-methylcyclopropoxy)-9-((4-methylpyridin-2-yl)methyl)-9H-purin-8-
yl)phenoxy)propan-2-one
2-(3-chloro-4-(9-(3-methoxybenzyl)-6-(1-methylcyclopropoxy)-9H-purin-8-
yl)phenyl)acetamide
2-(3-chloro-4-(9-(4-methoxybenzyl)-6-(1-methylcyclopropoxy)-9H-purin-8-
yl)phenyl)acetamide
1-(3-chloro-4-(6-(1-methylcyclopropoxy)-9-((4-methylpyridin-2-yl)methyl)-9H-purin-8-
yl)phenyl)imidazolidin-2-one
8-(2-chloro-4-(3-fluoropropoxy)phenyl)-6-(1-methylcyclopropoxy)-9-((4-methylpyridin-2-
yl)methyl)-9H-purine
3-(3-chloro-4-(6-(1-methylcyclopropoxy)-9-((4-methylpyridin-2-yl)methyl)-9H-purin-8-
yl)phenoxy)-2-fluoropropan-1-ol (Isomer 1)
3-(3-chloro-4-(6-(1-methylcyclopropoxy)-9-((4-methylpyridin-2-yl)methyl)-9H-purin-8-
yl)phenoxy)-2-fluoropropan-1-ol (Isomer 2)
8-(4-(azetidin-3-yloxy)-2-chlorophenyl)-6-(1-methylcyclopropoxy)-9-((4-methylpyridin-2-
yl)methyl)-9H-purine
3-chloro-N,N-dimethyl-4-(6-(1-methylcyclopropoxy)-9-((4-methylpyridin-2-yl)methyl)-9H-
purin-8-yl)benzamide
2-(3-chloro-4-(6-(1-methylcyclopropoxy)-9-((4-methylpyridin-2-yl)methyl)-9H-purin-8-
yl)phenyl)-N-methylacetamide
3-chloro-N-ethyl-4-(6-(1-methylcyclopropoxy)-9-((4-methylpyridin-2-yl)methyl)-9H-purin-8-
yl)benzamide
1-(3-chloro-4-(6-(1-methylcyclopropoxy)-9-((4-methylpyridin-2-yl)methyl)-9H-purin-8-
yl)phenyl)azetidin-3-ol
3-(3-chloro-4-(6-(1-methylcyclopropoxy)-9-((4-methylpyridin-2-yl)methyl)-9H-purin-8-
yl)phenyl)propanamide
2-(3-chloro-4-(6-(1-methylcyclopropoxy)-9-(2-(3-methylpyridin-2-yl)ethyl)-9H-purin-8-
yl)phenyl)acetamide
2-(3-chloro-4-(6-(1-methylcyclopropoxy)-9-((4-methylpyridin-2-yl)methyl)-9H-purin-8-
yl)phenoxy)-N-methylacetamide
3-chloro-N-(2-hydroxyethyl)-4-(6-(1-methylcyclopropoxy)-9-((4-methylpyridin-2-yl)methyl)-
9H-purin-8-yl)benzamide
2-(2-chloro-3-(6-(1-methylcyclopropoxy)-9-((4-methylpyridin-2-yl)methyl)-9H-purin-8-
yl)phenoxy)-N-methylacetamide
(R)-N-(3-chloro-4-(6-(1-methylcyclopropoxy)-9-((4-methylpyridin-2-yl)methyl)-9H-purin-8-
yl)phenyl)-2-hydroxypropanamide
2-(4-chloro-3-(6-(1-methylcyclopropoxy)-9-((4-methylpyridin-2-yl)methyl)-9H-purin-8-
yl)phenoxy)-N-methylacetamide
2-hydroxyethyl (3-chloro-4-(6-(1-methylcyclopropoxy)-9-((4-methylpyridin-2-yl)methyl)-9H-
purin-8-yl)phenyl)carbamate
3-(3-chloro-4-(6-(1-methylcyclopropoxy)-9-((4-methylpyridin-2-yl)methyl)-9H-purin-8-
yl)phenoxy)propan-1-ol
8-(2-chloro-4-(2-methoxyethoxy)phenyl)-6-(1-methylcyclopropoxy)-9-((4-methylpyridin-2-
yl)methyl)-9H-purine
2-(3-chloro-4-(6-(1-methylcyclopropoxy)-9-((4-methylpyridin-2-yl)methyl)-9H-purin-8-
yl)phenoxy)propan-1-ol (Isomer 2)
2-(3-chloro-4-(6-(1-methylcyclopropoxy)-9-((4-methylpyridin-2-yl)methyl)-9H-purin-8-
yl)phenoxy)propan-1-ol (Isomer 1)
8-(2-chloro-4-(2-(methylsulfonyl)ethoxy)phenyl)-6-(1-methylcyclopropoxy)-9-((4-
methylpyridin-2-yl)methyl)-9H-purine
3-(3-chloro-4-(6-(1-methylcyclopropoxy)-9-((4-methylpyridin-2-yl)methyl)-9H-purin-8-
yl)phenyl)-1,1-dimethylurea
2-((3-chloro-4-(6-(1-methylcyclopropoxy)-9-((4-methylpyridin-2-yl)methyl)-9H-purin-8-
yl)phenyl)amino)-N-methylacetamide
1-(3-chloro-4-(6-(1-methylcyclopropoxy)-9-((4-methylpyridin-2-yl)methyl)-9H-purin-8-
yl)phenyl)-3-(2-hydroxyethyl)urea
1-(2-(3-chloro-4-(6-(1-methylcyclopropoxy)-9-((4-methylpyridin-2-yl)methyl)-9H-purin-8-
yl)phenoxy)ethyl)urea
3-(3-chloro-4-(9-((4-chloropyridin-2-yl)methyl)-6-(1-methylcyclopropoxy)-9H-purin-8-
yl)phenoxy)-N-methylpropan-1-amine
2-(3-chloro-4-(9-((4-chloropyridin-2-yl)methyl)-6-(1-methylcyclopropoxy)-9H-purin-8-
yl)phenoxy)-N,N-dimethylethan-1-amine
1-(3-chloro-4-(6-(1-methylcyclopropoxy)-9-((4-methylpyridin-2-yl)methyl)-9H-purin-8-
yl)phenyl)-N,N-dimethylmethanamine
2-(3-chloro-4-(6-(1-methylcyclopropoxy)-9-((4-methylpyridin-2-yl)methyl)-9H-purin-8-
yl)phenoxy)-N-methylethan-1-amine
2-((8-(2-chloro-4-(piperidin-4-yloxy)phenyl)-6-(1-methylcyclopropoxy)-9H-purin-9-yl)methyl)-
5-methylthiazole
(R)-2-((8-(2-chloro-4-(pyrrolidin-2-ylmethoxy)phenyl)-6-(1-methylcyclopropoxy)-9H-purin-9-
yl)methyl)-5-methylthiazole
(S)-2-((8-(2-chloro-4-((1-methylpyrrolidin-3-yl)oxy)phenyl)-6-(1-methylcyclopropoxy)-9H-
purin-9-yl)methyl)-5-methylthiazole
1-(2-(3-chloro-4-(6-(1-methylcyclopropoxy)-9-((5-methylthiazol-2-yl)methyl)-9H-purin-8-
yl)phenoxy)ethyl)azetidin-3-ol
1-(2-(2-chloro-3-(6-(1-methylcyclopropoxy)-9-((5-methylthiazol-2-yl)methyl)-9H-purin-8-
yl)phenoxy)ethyl)azetidin-3-ol
N-(3-chloro-4-(6-(1-methylcyclopropoxy)-9-((4-methylpyridin-2-yl)methyl)-9H-purin-8-
yl)phenethyl)methanesulfonamide
N-(2-(3-chloro-4-(6-(1-methylcyclopropoxy)-9-((4-methylpyridin-2-yl)methyl)-9H-purin-8-
yl)phenoxy)ethyl)methanesulfonamide
5-chloro-4-((8-(2-chloro-4-(2-(piperazin-1-yl)ethoxy)phenyl)-6-(1-methylcyclopropoxy)-9H-
purin-9-yl)methyl)thiazole
2-chloro-5-((8-(2-chloro-4-(2-(piperazin-1-yl)ethoxy)phenyl)-6-(1-methylcyclopropoxy)-9H-
purin-9-yl)methyl)thiazole
2-(3-chloro-4-(6-(1-methylcyclopropoxy)-9-(2-(1,3,5-trimethyl-1H-pyrazol-4-yl)ethyl)-9H-
purin-8-yl)phenyl)acetamide
3-(3-chloro-4-(6-(1-methylcyclopropoxy)-9-((5-methylthiazol-2-yl)methyl)-9H-purin-8-
yl)phenoxy)-N,N-dimethylpropan-1-amine
3-(2-chloro-3-(6-(1-methylcyclopropoxy)-9-((5-methylthiazol-2-yl)methyl)-9H-purin-8-
yl)phenoxy)-N,N-dimethylpropan-1-amine
8-(2-chloro-4-(2-(piperazin-1-yl)ethoxy)phenyl)-9-((4-methyl-4H-1,2,4-triazol-3-yl)methyl)-6-
(1-methylcyclopropoxy)-9H-purine
3-(3-chloro-4-(6-(1-methylcyclopropoxy)-9-(3-(trifluoromethyl)benzyl)-9H-purin-8-
yl)phenoxy)propanoic acid
3-chloro-4-(6-(1-methylcyclopropoxy)-9-((4-methylpyridin-2-yl)methyl)-9H-purin-8-yl)-N-
(prop-2-yn-1-yl)benzamide
(3-chloro-4-(6-(1-methylcyclopropoxy)-9-((4-methylpyridin-2-yl)methyl)-9H-purin-8-
yl)phenyl)(3-fluoroazetidin-1-yl)methanone
2-(3-chloro-4-(6-(1-methylcyclopropoxy)-9-((4-methylpyridin-2-yl)methyl)-9H-purin-8-
yl)phenoxy)-N-(2,2,2-trifluoroethyl)acetamide
4-(3-chloro-4-(9-(3-chlorobenzyl)-6-(1-methylcyclopropoxy)-9H-purin-8-yl)phenyl)butanoic
acid
methyl 3-(3-chloro-4-(9-(3-chlorobenzyl)-6-(1-methylcyclopropoxy)-9H-purin-8-
yl)phenoxy)propanoate
4-(2-chloro-3-(9-(3-chlorobenzyl)-6-(1-methylcyclopropoxy)-9H-purin-8-yl)phenoxy)butanoic
acid
2-(2-chloro-3-(9-(3-chlorobenzyl)-6-(1-methylcyclopropoxy)-9H-purin-8-yl)phenoxy)-2-
methylpropanoic acid
(R)-3-(2-chloro-3-(9-(3-chlorobenzyl)-6-(1-methylcyclopropoxy)-9H-purin-8-yl)phenoxy)-2-
methylpropanoic acid
(S)-3-(2-chloro-3-(9-(3-chlorobenzyl)-6-(1-methylcyclopropoxy)-9H-purin-8-yl)phenoxy)-2-
methylpropanoic acid
2-(2-(2-chloro-3-(9-(3-chlorobenzyl)-6-(1-methylcyclopropoxy)-9H-purin-8-
yl)phenoxy)ethoxy)acetic acid
8-(3-(3-(2H-tetrazol-5-yl)propoxy)-2-chlorophenyl)-9-(3-chlorobenzyl)-6-(1-
methylcyclopropoxy)-9H-purine
8-(4-(3-(1H-tetrazol-5-yl)propoxy)-2-chlorophenyl)-9-(3-chlorobenzyl)-6-(1-
methylcyclopropoxy)-9H-purine
2-(3-chloro-4-(6-(1-methylcyclopropoxy)-9-((4-methylpyridin-2-yl)methyl)-9H-purin-8-
yl)phenyl)-N-(2,2-difluoroethyl)acetamide
2-(3-chloro-4-(6-(1-methylcyclopropoxy)-9-((4-methylpyridin-2-yl)methyl)-9H-purin-8-
yl)phenoxy)-N-(2,2-difluoroethyl)acetamide
3-(3-chloro-4-(9-(3-methylbenzyl)-6-(1-methylcyclopropoxy)-9H-purin-8-
yl)phenoxy)propanoic acid
3-(3-chloro-4-(9-(2-methylbenzyl)-6-(1-methylcyclopropoxy)-9H-purin-8-
yl)phenoxy)propanoic acid
azetidin-1-yl(3-chloro-4-(6-(1-methylcyclopropoxy)-9-((4-methylpyridin-2-yl)methyl)-9H-
purin-8-yl)phenyl)methanone
3-chloro-N-cyclopropyl-4-(6-(1-methylcyclopropoxy)-9-((4-methylpyridin-2-yl)methyl)-9H-
purin-8-yl)benzamide
1-(3-chloro-4-(6-(1-methylcyclopropoxy)-9-((4-methylpyridin-2-yl)methyl)-9H-purin-8-
yl)phenyl)pyrrolidin-2-one
5-(3-chloro-4-(6-(1-methylcyclopropoxy)-9-((4-methylpyridin-2-yl)methyl)-9H-purin-8-
yl)phenyl)pyrrolidin-2-one (Isomer 1)
5-(3-chloro-4-(6-(1-methylcyclopropoxy)-9-((4-methylpyridin-2-yl)methyl)-9H-purin-8-
yl)phenyl)pyrrolidin-2-one (Isomer 2)
4-(3-chloro-4-(6-(1-methylcyclopropoxy)-9-((4-methylpyridin-2-yl)methyl)-9H-purin-8-
yl)phenyl)pyrrolidin-2-one (Isomer 1)
4-(3-chloro-4-(6-(1-methylcyclopropoxy)-9-((4-methylpyridin-2-yl)methyl)-9H-purin-8-
yl)phenyl)pyrrolidin-2-one (Isomer 2)
3-(3-chloro-4-(6-(1-methylcyclopropoxy)-9-((4-methylpyridin-2-yl)methyl)-9H-purin-8-
yl)phenyl)pyrrolidin-2-one (racemic)
3-chloro-4-(6-(1-methylcyclopropoxy)-9-((4-methylpyridin-2-yl)methyl)-9H-purin-8-yl)-N-
(oxetan-3-yl)benzamide
3-(3-chloro-4-(6-(1-methylcyclopropoxy)-9-((4-methylpyridin-2-yl)methyl)-9H-purin-8-
yl)phenoxy)pyrrolidin-2-one (racemic)
4-(3-chloro-4-(6-(1-methylcyclopropoxy)-9-((4-methylpyridin-2-yl)methyl)-9H-purin-8-
yl)phenoxy)pyrrolidin-2-one (racemic)
(3-chloro-4-(6-(1-methylcyclopropoxy)-9-((4-methylpyridin-2-yl)methyl)-9H-purin-8-
yl)phenyl)(3-hydroxyazetidin-1-yl)methanone
(R)-1-(3-chloro-4-(6-(1-methylcyclopropoxy)-9-((4-methylpyridin-2-yl)methyl)-9H-purin-8-
yl)phenyl)-3-hydroxypyrrolidin-2-one
4-(3-chloro-4-(6-(1-methylcyclopropoxy)-9-((4-methylpyridin-2-yl)methyl)-9H-purin-8-
yl)phenyl)morpholin-3-one
(R)-4-(3-chloro-4-(6-(1-methylcyclopropoxy)-9-((4-methylpyridin-2-yl)methyl)-9H-purin-8-
yl)phenoxy)pyrrolidin-2-one
(S)-4-(3-chloro-4-(6-(1-methylcyclopropoxy)-9-((4-methylpyridin-2-yl)methyl)-9H-purin-8-
yl)phenoxy)pyrrolidin-2-one
(S)-3-(3-chloro-4-(6-(1-methylcyclopropoxy)-9-((4-methylpyridin-2-yl)methyl)-9H-purin-8-
yl)phenoxy)pyrrolidin-2-one
(R)-3-(3-chloro-4-(6-(1-methylcyclopropoxy)-9-((4-methylpyridin-2-yl)methyl)-9H-purin-8-
yl)phenoxy)pyrrolidin-2-one
5-(3-chloro-4-(6-(1-methylcyclopropoxy)-9-((4-methylpyridin-2-yl)methyl)-9H-purin-8-
yl)phenoxy)-1,3-oxazinan-2-one (racemic)
(R)-5-((3-chloro-4-(6-(1-methylcyclopropoxy)-9-((4-methylpyridin-2-yl)methyl)-9H-purin-8-
yl)phenoxy)methyl)oxazolidin-2-one
3-(3-chloro-4-(9-(3-chlorobenzyl)-6-(1-methylcyclopropoxy)-9H-purin-8-yl)phenoxy)-N-
(methylsulfonyl)propanamide
(S)-3-chloro-N-(2-fluoropropyl)-4-(6-(1-methylcyclopropoxy)-9-((4-methylpyridin-2-
yl)methyl)-9H-purin-8-yl)benzamide
3-chloro-N-(3-fluoropropyl)-4-(6-(1-methylcyclopropoxy)-9-((4-methylpyridin-2-yl)methyl)-
9H-purin-8-yl)benzamide
1-(4-(2-(4-(9-((1,3,4-oxadiazol-2-yl)methyl)-6-(1-methylcyclopropoxy)-9H-purin-8-yl)-3-
chlorophenoxy)ethyl)piperazin-1-yl)-2,2,2-trifluoroethan-1-one
8-(2-chloro-4-(oxetan-3-ylmethoxy)phenyl)-6-(1-methylcyclopropoxy)-9-((4-methylpyridin-2-
yl)methyl)-9H-purine
(R)-8-(2-chloro-4-(oxetan-2-ylmethoxy)phenyl)-6-(1-methylcyclopropoxy)-9-((4-
methylpyridin-2-yl)methyl)-9H-purine
2-(3-chloro-4-(6-(1-methylcyclopropoxy)-9-phenethyl-9H-purin-8-yl)phenoxy)-N-
methylacetamide
(S)-8-(2-chloro-4-((tetrahydrofuran-3-yl)oxy)phenyl)-6-(1-methylcyclopropoxy)-9-((4-
methylpyridin-2-yl)methyl)-9H-purine
1-((3-chloro-4-(6-(1-methylcyclopropoxy)-9-((4-methylpyridin-2-yl)methyl)-9H-purin-8-
yl)phenoxy)methyl)cyclopropan-1-ol
(1s,3s)-3-(3-chloro-4-(6-(1-methylcyclopropoxy)-9-((4-methylpyridin-2-yl)methyl)-9H-purin-8-
yl)phenoxy)cyclobutan-1-ol
(1r,3r)-3-(3-chloro-4-(6-(1-methylcyclopropoxy)-9-((4-methylpyridin-2-yl)methyl)-9H-purin-8-
yl)phenoxy)cyclobutan-1-ol
3-((3-chloro-4-(6-(1-methylcyclopropoxy)-9-((4-methylpyridin-2-yl)methyl)-9H-purin-8-
yl)phenoxy)methyl)oxetan-3-ol
4-(3-chloro-4-(6-(1-methylcyclopropoxy)-9-((4-methylpyridin-2-yl)methyl)-9H-purin-8-
yl)phenyl)piperazin-2-one
1-(3-chloro-4-(6-(1-methylcyclopropoxy)-9-((4-methylpyridin-2-yl)methyl)-9H-purin-8-
yl)phenyl)piperazin-2-one
1-((8-(2-chloro-4-(2-(dimethylamino)ethoxy)phenyl)-9-((4-methylpyridin-2-yl)methyl)-9H-
purin-6-yl)oxy)cyclopropane-1-carbonitrile
1-(3-chloro-4-(6-(1-methylcyclopropoxy)-9-((4-methylpyridin-2-yl)methyl)-9H-purin-8-
yl)phenyl)azetidine-3-carboxamide
3-(3-chloro-4-(6-(1-methylcyclopropoxy)-9-((4-methylpyridin-2-yl)methyl)-9H-purin-8-
yl)phenoxy)azetidine-1-carboxamide
1-(2-(3-chloro-4-(9-((4-chloropyridin-2-yl)methyl)-6-(1-methylcyclopropoxy)-9H-purin-8-
yl)phenoxy)ethyl)azetidin-3-ol
2-((8-(2-chloro-4-(2-(piperazin-1-yl)ethoxy)phenyl)-6-(1-methylcyclopropoxy)-9H-purin-9-
yl)methyl)-5-(trifluoromethyl)thiazole
(S)-8-(2-chloro-4-(pyrrolidin-3-yloxy)phenyl)-6-(1-methylcyclopropoxy)-9-((4-methylpyridin-
2-yl)methyl)-9H-purine
1-(3-chloro-4-(6-(1-methylcyclopropoxy)-9-((4-methylpyridin-2-yl)methyl)-9H-purin-8-
yl)benzyl)azetidin-3-ol
2-(3-chloro-4-(6-(1-methylcyclopropoxy)-9-((4-methylpyridin-2-yl)methyl)-9H-purin-8-
yl)phenyl)-N,N-dimethylacetamide
3-chloro-N-isopropyl-4-(6-(1-methylcyclopropoxy)-9-((4-methylpyridin-2-yl)methyl)-9H-
purin-8-yl)benzamide
N-(3-chloro-4-(6-(1-methylcyclopropoxy)-9-((4-methylpyridin-2-yl)methyl)-9H-purin-8-
yl)phenethyl)acetamide
2-(3-chloro-4-(6-(1-methylcyclopropoxy)-9-((4-methylpyridin-2-yl)methyl)-9H-purin-8-
yl)phenyl)-N-ethylacetamide
3-(3-chloro-4-(6-(1-methylcyclopropoxy)-9-((4-methylpyridin-2-yl)methyl)-9H-purin-8-
yl)phenyl)-N-methylpropanamide
2-((8-(4-((2-azaspiro[3.3]heptan-6-yl)oxy)-2-chlorophenyl)-6-(1-methylcyclopropoxy)-9H-
purin-9-yl)methyl)-5-methylthiazole
2-(3-chloro-4-(6-(1-methylcyclopropoxy)-9-((4-methylpyridin-2-yl)methyl)-9H-purin-8-
yl)phenoxy)-N,N-dimethylacetamide
3-chloro-N-(3-hydroxypropyl)-4-(6-(1-methylcyclopropoxy)-9-((4-methylpyridin-2-yl)methyl)-
9H-purin-8-yl)benzamide
(S)-3-chloro-N-(2-hydroxypropyl)-4-(6-(1-methylcyclopropoxy)-9-((4-methylpyridin-2-
yl)methyl)-9H-purin-8-yl)benzamide
N-(2-(3-chloro-4-(6-(1-methylcyclopropoxy)-9-((4-methylpyridin-2-yl)methyl)-9H-purin-8-
yl)phenoxy)ethyl)acetamide
3-chloro-N-(2-hydroxyethyl)-N-methyl-4-(6-(1-methylcyclopropoxy)-9-((4-methylpyridin-2-
yl)methyl)-9H-purin-8-yl)benzamide
2-(3-chloro-4-(6-(1-methylcyclopropoxy)-9-((4-methylpyridin-2-yl)methyl)-9H-purin-8-
yl)phenoxy)-N-ethylacetamide
methyl (3-chloro-4-(6-(1-methylcyclopropoxy)-9-((4-methylpyridin-2-yl)methyl)-9H-purin-8-
yl)phenethyl)carbamate
N-(3-chloro-4-(6-(1-methylcyclopropoxy)-9-((4-methylpyridin-2-yl)methyl)-9H-purin-8-
yl)phenethyl)-2-hydroxyacetamide
2-(3-chloro-4-(6-(1-methylcyclopropoxy)-9-((4-methylpyridin-2-yl)methyl)-9H-purin-8-
yl)phenyl)-N-(2-hydroxyethyl)acetamide
4-(3-chloro-4-(6-(1-methylcyclopropoxy)-9-((4-methylpyridin-2-yl)methyl)-9H-purin-8-
yl)phenyl)thiomorpholine 1,1-dioxide
2-(3-chloro-4-(6-(1-methylcyclopropoxy)-9-((4-methylpyridin-2-yl)methyl)-9H-purin-8-
yl)phenoxy)-N-(2-hydroxyethyl)acetamide
N-(2-(3-chloro-4-(6-(1-methylcyclopropoxy)-9-((4-methylpyridin-2-yl)methyl)-9H-purin-8-
yl)phenoxy)ethyl)-2-hydroxyacetamide
methyl (2-(3-chloro-4-(6-(1-methylcyclopropoxy)-9-((4-methylpyridin-2-yl)methyl)-9H-purin-
8-yl)phenoxy)ethyl)carbamate
1-(3-chloro-4-(9-((4-chloropyridin-2-yl)methyl)-6-(1-methylcyclopropoxy)-9H-purin-8-
yl)phenyl)piperidin-4-amine
3-(4-(9-((4-chloropyridin-2-yl)methyl)-6-(1-methylcyclopropoxy)-9H-purin-8-yl)-3-
fluorophenoxy)-N,N-dimethylpropan-1-amine
8-(4-(sec-butyl)-2-chlorophenyl)-6-(1-methylcyclopropoxy)-9-((4-methylpyridin-2-yl)methyl)-
9H-purine (racemic)
2-(2-(3-chloro-4-(6-(1-methylcyclopropoxy)-9-((4-methylpyridin-2-yl)methyl)-9H-purin-8-
yl)phenoxy)ethoxy)ethan-1-ol
8-(2-chloro-4-(3-(methylsulfonyl)propoxy)phenyl)-6-(1-methylcyclopropoxy)-9-((4-
methylpyridin-2-yl)methyl)-9H-purine
8-(2-chloro-4-(piperazin-1-yl)phenyl)-6-(1-methylcyclopropoxy)-9-((4-methylpyridin-2-
yl)methyl)-9H-purine
2-(3-chloro-4-(6-(1-methylcyclopropoxy)-9-((4-methylpyridin-2-yl)methyl)-9H-purin-8-
yl)phenyl)-N,N-dimethylethan-1-amine
2-(2-chloro-3-(6-(1-methylcyclopropoxy)-9-((4-methylpyridin-2-yl)methyl)-9H-purin-8-
yl)phenoxy)-N,N-dimethylethan-1-amine
3-(3-chloro-4-(6-(1-methylcyclopropoxy)-9-((4-methylpyridin-2-yl)methyl)-9H-purin-8-
yl)phenoxy)-N-methylpropan-1-amine
2-((8-(2-chloro-4-(2-(pyrrolidin-1-yl)ethoxy)phenyl)-6-(1-methylcyclopropoxy)-9H-purin-9-
yl)methyl)-5-methylthiazole
(R)-2-((8-(2-chloro-4-((1-methylpyrrolidin-2-yl)methoxy)phenyl)-6-(1-methylcyclopropoxy)-
9H-purin-9-yl)methyl)-5-methylthiazole
2-((8-(2-chloro-4-((1-methylpiperidin-4-yl)oxy)phenyl)-6-(1-methylcyclopropoxy)-9H-purin-9-
yl)methyl)-5-methylthiazole
2-((8-(4-(azepan-4-yloxy)-2-chlorophenyl)-6-(1-methylcyclopropoxy)-9H-purin-9-yl)methyl)-5-
methylthiazole (isomer 2)
2-((8-(4-(azepan-4-yloxy)-2-chlorophenyl)-6-(1-methylcyclopropoxy)-9H-purin-9-yl)methyl)-5-
methylthiazole (isomer 1)
1-(3-(3-chloro-4-(6-(1-methylcyclopropoxy)-9-((5-methylthiazol-2-yl)methyl)-9H-purin-8-
yl)phenoxy)propyl)azetidin-3-ol
(R)-1-(2-(3-chloro-4-(6-(1-methylcyclopropoxy)-9-((5-methylthiazol-2-yl)methyl)-9H-purin-8-
yl)phenoxy)ethyl)pyrrolidin-3-ol
1-(3-(2-chloro-3-(6-(1-methylcyclopropoxy)-9-((5-methylthiazol-2-yl)methyl)-9H-purin-8-
yl)phenoxy)propyl)azetidin-3-ol
N1-(3-chloro-4-(6-(1-methylcyclopropoxy)-9-((4-methylpyridin-2-yl)methyl)-9H-purin-8-
yl)phenyl)-N2,N2-dimethylethane-1,2-diamine
2-((8-(2-chloro-4-(2-(piperazin-1-yl)ethoxy)phenyl)-6-(1-methylcyclopropoxy)-9H-purin-9-
yl)methyl)-4-methylthiazole
5-((8-(2-chloro-4-(2-(piperazin-1-yl)ethoxy)phenyl)-6-(1-methylcyclopropoxy)-9H-purin-9-
yl)methyl)-2-methylthiazole
2-((3-chloro-4-(9-(3-chlorobenzyl)-6-(1-methylcyclopropoxy)-9H-purin-8-
yl)phenoxy)methyl)thiazole-4-carboxylic acid
4-(3-chloro-4-(6-(1-methylcyclopropoxy)-9-((perfluorophenyl)methyl)-9H-purin-8-
yl)phenoxy)-2-methylbutanoic acid (racemic)
4-(3-chloro-4-(9-(3-chlorobenzyl)-6-(1-cyanocyclopropoxy)-9H-purin-8-yl)phenoxy)-2-
methylbutanoic acid (racemic)
(1r,3r)-3-(3-chloro-4-(9-(3-chlorobenzyl)-6-(1-methylcyclopropoxy)-9H-purin-8-
yl)phenoxy)cyclobutane-1-carboxylic acid
(1s,3s)-3-(3-chloro-4-(9-(3-chlorobenzyl)-6-(1-methylcyclopropoxy)-9H-purin-8-
yl)phenoxy)cyclobutane-1-carboxylic acid
(E)-4-(3-chloro-4-(9-(3-chlorobenzyl)-6-(1-methylcyclopropoxy)-9H-purin-8-yl)phenoxy)-2-
methylbut-2-enoic acid
(1r,3r)-3-(2-chloro-3-(9-(3-chlorobenzyl)-6-(1-methylcyclopropoxy)-9H-purin-8-
yl)phenoxy)cyclobutane-1-carboxylic acid
(1s,3s)-3-(2-chloro-3-(9-(3-chlorobenzyl)-6-(1-methylcyclopropoxy)-9H-purin-8-
yl)phenoxy)cyclobutane-1-carboxylic acid
4-(4-(9-(5-bromo-2-fluorobenzyl)-6-(1-methylcyclopropoxy)-9H-purin-8-yl)-3-chlorophenoxy)-
2-methylbutanoic acid (racemic)
4-(4-(9-(2-bromo-6-chlorobenzyl)-6-(1-methylcyclopropoxy)-9H-purin-8-yl)-3-
chlorophenoxy)-2-methylbutanoic acid (racemic)
4-(3-chloro-4-(9-(3,5-difluorobenzyl)-6-(1-methylcyclopropoxy)-9H-purin-8-yl)phenoxy)-2-
methylbutanoic acid (racemic)
4-(3-chloro-4-(9-(2,3-difluorobenzyl)-6-(1-methylcyclopropoxy)-9H-purin-8-yl)phenoxy)-2-
methylbutanoic acid (Isomer 1)
4-(3-chloro-4-(9-(2,3-difluorobenzyl)-6-(1-methylcyclopropoxy)-9H-purin-8-yl)phenoxy)-2-
methylbutanoic acid (Isomer 2)
4-(3-chloro-4-(9-(2,6-difluorobenzyl)-6-(1-methylcyclopropoxy)-9H-purin-8-yl)phenoxy)-2-
methylbutanoic acid (isomer 1)
4-(3-chloro-4-(9-(2,6-difluorobenzyl)-6-(1-methylcyclopropoxy)-9H-purin-8-yl)phenoxy)-2-
methylbutanoic acid (isomer 2)
2-(3-chloro-4-(6-(1-methylcyclopropoxy)-9-((4-methylpyridin-2-yl)methyl)-9H-purin-8-
yl)phenoxy)-N-(prop-2-yn-1-yl)acetamide
4-(3-chloro-4-(9-(3-chloro-2-fluorobenzyl)-6-(1-methylcyclopropoxy)-9H-purin-8-yl)phenoxy)-
2-methylbutanoic acid (racemic)
4-(3-chloro-4-(9-(5-chloro-2-fluorobenzyl)-6-(1-methylcyclopropoxy)-9H-purin-8-yl)phenoxy)-
2-methylbutanoic acid (racemic)
4-(3-chloro-4-(9-(3-chloro-4-fluorobenzyl)-6-(1-methylcyclopropoxy)-9H-purin-8-yl)phenoxy)-
2-methylbutanoic acid (racemic)
4-(3-chloro-4-(9-(3-chloro-2-fluorobenzyl)-6-(1-methylcyclopropoxy)-9H-purin-8-yl)phenoxy)-
2-methylbutanoic acid (Isomer 1)
4-(3-chloro-4-(9-(3-chloro-2-fluorobenzyl)-6-(1-methylcyclopropoxy)-9H-purin-8-yl)phenoxy)-
2-methylbutanoic acid (Isomer 2)
4-(3-chloro-4-(9-(3-chloro-5-fluorobenzyl)-6-(1-methylcyclopropoxy)-9H-purin-8-yl)phenoxy)-
2-methylbutanoic acid (isomer 1)
4-(3-chloro-4-(9-(3-chloro-5-fluorobenzyl)-6-(1-methylcyclopropoxy)-9H-purin-8-yl)phenoxy)-
2-methylbutanoic acid (isomer 2)
4-(3-chloro-4-(9-(3-chlorobenzyl)-6-((1,1,1-trifluoro-2-methylpropan-2-yl)oxy)-9H-purin-8-
yl)phenoxy)-2-methylbutanoic acid (racemic)
4-(3-chloro-4-(9-(3,5-dichlorobenzyl)-6-(1-methylcyclopropoxy)-9H-purin-8-yl)phenoxy)-2-
methylbutanoic acid (racemic)
4-(3-chloro-4-(9-(2,5-dichlorobenzyl)-6-(1-methylcyclopropoxy)-9H-purin-8-yl)phenoxy)-2-
methylbutanoic acid (racemic)
4-(3-chloro-4-(9-(2,6-dichlorobenzyl)-6-(1-methylcyclopropoxy)-9H-purin-8-yl)phenoxy)-2-
methylbutanoic acid (racemic)
4-(4-(9-(2-bromobenzyl)-6-(1-methylcyclopropoxy)-9H-purin-8-yl)-3-chlorophenoxy)-2-
methylbutanoic acid (racemic)
4-(4-(9-(3-bromobenzyl)-6-(1-methylcyclopropoxy)-9H-purin-8-yl)-3-chlorophenoxy)-2-
methylbutanoic acid (racemic)
3-chloro-N-(3-fluorocyclobutyl)-4-(6-(1-methylcyclopropoxy)-9-((4-methylpyridin-2-
yl)methyl)-9H-purin-8-yl)benzamide
(S)-(3-chloro-4-(6-(1-methylcyclopropoxy)-9-((4-methylpyridin-2-yl)methyl)-9H-purin-8-
yl)phenyl)(3-fluoropyrrolidin-1-yl)methanone
(3-chloro-4-(6-(1-methylcyclopropoxy)-9-((4-methylpyridin-2-yl)methyl)-9H-purin-8-
yl)phenyl)((3R,4R)-3-fluoro-4-hydroxypyrrolidin-1-yl)methanone
2-(3-chloro-4-(6-(1-methylcyclopropoxy)-9-((4-methylpyridin-2-yl)methyl)-9H-purin-8-
yl)phenoxy)-1-(3-fluoroazetidin-1-yl)ethan-1-one
1-(2-(3-chloro-4-(6-(1-methylcyclopropoxy)-9-((4-(trifluoromethyl)pyridin-2-yl)methyl)-9H-
purin-8-yl)phenoxy)ethyl)azetidin-3-ol
4-(3-chloro-4-(9-(3-iodobenzyl)-6-(1-methylcyclopropoxy)-9H-purin-8-yl)phenoxy)-2-
methylbutanoic acid (racemic)
3-chloro-N-(2-cyanoethyl)-N-methyl-4-(6-(1-methylcyclopropoxy)-9-((4-methylpyridin-2-
yl)methyl)-9H-purin-8-yl)benzamide
4-(3-chloro-4-(6-(1-methylcyclopropoxy)-9-((4-methylpyridin-2-yl)methyl)-9H-purin-8-
yl)benzoyl)piperazin-2-one
3-(4-(4-(tert-butoxy)-1-(3-chlorobenzyl)-1H-benzo[d]imidazol-2-yl)-3-
chlorophenoxy)propanoic acid
5-(3-chloro-4-(9-(3-chlorobenzyl)-6-(1-methylcyclopropoxy)-9H-purin-8-yl)phenyl)pentanoic
acid
4-(3-chloro-4-(9-(3-chlorobenzyl)-6-(1-methylcyclopropoxy)-9H-purin-8-yl)phenyl)-2-
methylbutanoic acid (racemic)
4-(3-chloro-4-(9-(3-chlorobenzyl)-6-(1-methylcyclopropoxy)-9H-purin-8-yl)phenoxy)-2-
methylbutanoic acid (racemic)
5-(2-chloro-3-(9-(3-chlorobenzyl)-6-(1-methylcyclopropoxy)-9H-purin-8-yl)phenoxy)pentanoic
acid
4-(2-chloro-3-(9-(3-chlorobenzyl)-6-(1-methylcyclopropoxy)-9H-purin-8-yl)phenoxy)-2-
methylbutanoic acid (racemic)
4-(3-chloro-4-(9-(3-chlorobenzyl)-6-(1-methylcyclopropoxy)-9H-purin-8-yl)phenoxy)-3-
methylbutanoic acid (Isomer 1)
4-(3-chloro-4-(9-(3-chlorobenzyl)-6-(1-methylcyclopropoxy)-9H-purin-8-yl)phenoxy)-3-
methylbutanoic acid (Isomer 2)
(S)-4-(2-chloro-3-(9-(3-chlorobenzyl)-6-(1-methylcyclopropoxy)-9H-purin-8-yl)phenoxy)-2-
methylbutanoic acid
(R)-4-(2-chloro-3-(9-(3-chlorobenzyl)-6-(1-methylcyclopropoxy)-9H-purin-8-yl)phenoxy)-2-
methylbutanoic acid
3-(2-chloro-3-(9-(3-chlorobenzyl)-6-(1-methylcyclopropoxy)-9H-purin-8-yl)phenoxy)-2,2-
dimethylpropanoic acid
4-(2-chloro-3-(9-(3-chlorobenzyl)-6-(1-methylcyclopropoxy)-9H-purin-8-yl)phenoxy)-3-
methylbutanoic acid (Isomer 2)
4-(2-chloro-3-(9-(3-chlorobenzyl)-6-(1-methylcyclopropoxy)-9H-purin-8-yl)phenoxy)-3-
methylbutanoic acid (Isomer 1)
(R)-4-(3-chloro-4-(9-(3-chlorobenzyl)-6-(1-methylcyclopropoxy)-9H-purin-8-yl)phenoxy)-2-
hydroxy-2-methylbutanoic acid
2-(3-(3-chloro-4-(9-(3-chlorobenzyl)-6-(1-methylcyclopropoxy)-9H-purin-8-
yl)phenoxy)propoxy)acetic acid
3-(2-(3-chloro-4-(9-(3-chlorobenzyl)-6-(1-methylcyclopropoxy)-9H-purin-8-
yl)phenoxy)ethoxy)propanoic acid
2-(3-(2-chloro-3-(9-(3-chlorobenzyl)-6-(1-methylcyclopropoxy)-9H-purin-8-
yl)phenoxy)propoxy)acetic acid
rac-(R)-2-(2-(3-chloro-4-(9-(3-chlorobenzyl)-6-(1-methylcyclopropoxy)-9H-purin-8-
yl)phenoxy)ethoxy)propanoic acid
(R)-2-(2-(3-chloro-4-(9-(3-chlorobenzyl)-6-(1-methylcyclopropoxy)-9H-purin-8-
yl)phenoxy)ethoxy)propanoic acid
1-(2-(3-chloro-4-(9-((4-(difluoromethyl)pyridin-2-yl)methyl)-6-(1-methylcyclopropoxy)-9H-
purin-8-yl)phenoxy)ethyl)azetidin-3-ol
1-(azetidin-1-yl)-2-(3-chloro-4-(6-(1-methylcyclopropoxy)-9-((4-methylpyridin-2-yl)methyl)-
9H-purin-8-yl)phenyl)ethan-1-one
(3-chloro-4-(6-(1-methylcyclopropoxy)-9-((4-methylpyridin-2-yl)methyl)-9H-purin-8-
yl)phenyl)(pyrrolidin-1-yl)methanone
2-(3-chloro-4-(6-(1-methylcyclopropoxy)-9-((4-methylpyridin-2-yl)methyl)-9H-purin-8-
yl)phenyl)-N-cyclopropylacetamide
(R)-(3-chloro-4-(6-(1-methylcyclopropoxy)-9-((4-methylpyridin-2-yl)methyl)-9H-purin-8-
yl)phenyl)(3-hydroxypyrrolidin-1-yl)methanone
1-(azetidin-1-yl)-2-(3-chloro-4-(6-(1-methylcyclopropoxy)-9-((4-methylpyridin-2-yl)methyl)-
9H-purin-8-yl)phenoxy)ethan-1-one
1-(2-(3-chloro-4-(6-(1-methylcyclopropoxy)-9-((4-methylpyridin-2-yl)methyl)-9H-purin-8-
yl)phenoxy)ethyl)azetidin-2-one
(3-chloro-4-(6-(1-methylcyclopropoxy)-9-((4-methylpyridin-2-yl)methyl)-9H-purin-8-
yl)phenyl)(3-hydroxy-3-methylazetidin-1-yl)methanone
(3-chloro-4-(6-(1-methylcyclopropoxy)-9-((4-methylpyridin-2-yl)methyl)-9H-purin-8-
yl)phenyl)(morpholino)methanone
(R)-3-chloro-4-(6-(1-methylcyclopropoxy)-9-((4-methylpyridin-2-yl)methyl)-9H-purin-8-yl)-N-
(tetrahydrofuran-3-yl)benzamide
3-chloro-4-(6-(1-methylcyclopropoxy)-9-((4-methylpyridin-2-yl)methyl)-9H-purin-8-yl)-N-(3-
methyloxetan-3-yl)benzamide
2-(3-chloro-4-(6-(1-methylcyclopropoxy)-9-((4-methylpyridin-2-yl)methyl)-9H-purin-8-
yl)phenoxy)-N-cyclopropylacetamide
3-chloro-N-(3-hydroxycyclobutyl)-4-(6-(1-methylcyclopropoxy)-9-((4-methylpyridin-2-
yl)methyl)-9H-purin-8-yl)benzamide
3-chloro-N-methyl-4-(6-(1-methylcyclopropoxy)-9-((4-methylpyridin-2-yl)methyl)-9H-purin-8-
yl)-N-(oxetan-3-yl)benzamide
1-(3-chloro-4-(6-(1-methylcyclopropoxy)-9-((4-methylpyridin-2-yl)methyl)-9H-purin-8-
yl)phenyl)-4-hydroxypiperidin-2-one (racemic)
2-(3-chloro-4-(6-(1-methylcyclopropoxy)-9-((4-methylpyridin-2-yl)methyl)-9H-purin-8-
yl)phenoxy)-1-(3-hydroxyazetidin-1-yl)ethan-1-one
2-(3-chloro-4-(6-(1-methylcyclopropoxy)-9-((4-methylpyridin-2-yl)methyl)-9H-purin-8-
yl)phenoxy)-N-(oxetan-3-yl)acetamide
(S)-6-((3-chloro-4-(6-(1-methylcyclopropoxy)-9-((4-methylpyridin-2-yl)methyl)-9H-purin-8-
yl)phenoxy)methyl)morpholin-3-one
4-(2-chloro-3-(9-(3-chlorobenzyl)-6-(1-methylcyclopropoxy)-9H-purin-8-yl)phenoxy)-N-
(methylsulfonyl)butanamide
4-(3-chloro-4-(9-(3-chlorobenzyl)-6-(1-methylcyclopropoxy)-9H-purin-8-yl)phenoxy)-N-
(methylsulfonyl)butanamide
(S)-2-(3-chloro-4-(6-(1-methylcyclopropoxy)-9-((4-methylpyridin-2-yl)methyl)-9H-purin-8-
yl)phenoxy)-N-(2-fluoropropyl)acetamide
2-(3-chloro-4-(6-(1-methylcyclopropoxy)-9-((4-methylpyridin-2-yl)methyl)-9H-purin-8-
yl)phenoxy)-N-(3-fluoropropyl)acetamide
3-(3-chloro-4-(6-(1-methylcyclopropoxy)-9-((4-(trifluoromethyl)pyridin-2-yl)methyl)-9H-
purin-8-yl)phenoxy)-N,N-dimethylpropan-1-amine
rel-(1R,3R)-3-(3-chloro-4-(6-(1-methylcyclopropoxy)-9-((4-methylpyridin-2-yl)methyl)-9H-
purin-8-yl)phenoxy)cyclopentan-1-ol (Isomer 1)
8-(2-chloro-4-((3-methoxyoxetan-3-yl)methoxy)phenyl)-6-(1-methylcyclopropoxy)-9-((4-
methylpyridin-2-yl)methyl)-9H-purine
3-(2-(3-chloro-4-(6-(1-methylcyclopropoxy)-9-((4-methylpyridin-2-yl)methyl)-9H-purin-8-
yl)phenoxy)ethyl)oxetan-3-ol
4-(3-chloro-4-(6-(1-methylcyclopropoxy)-9-((4-methylpyridin-2-yl)methyl)-9H-purin-8-
yl)phenoxy)tetrahydro-2H-thiopyran 1,1-dioxide
8-(4-((1R,4R)-2,5-diazabicyclo[2.2.1]heptan-2-yl)-2-chlorophenyl)-6-(1-
methylcyclopropoxy)-9-((4-methylpyridin-2-yl)methyl)-9H-purine
(3-chloro-4-(6-(1-methylcyclopropoxy)-9-((4-methylpyridin-2-yl)methyl)-9H-purin-8-
yl)phenyl)(piperazin-1-yl)methanone
4-(3-chloro-4-(6-(1-methylcyclopropoxy)-9-((4-methylpyridin-2-yl)methyl)-9H-purin-8-
yl)phenyl)-1,4-diazepan-5-one
1-(3-chloro-4-(6-(1-methylcyclopropoxy)-9-((4-methylpyridin-2-yl)methyl)-9H-purin-8-
yl)phenyl)-1,4-diazepan-5-one
(S)-1-(3-chloro-4-(6-(1-methylcyclopropoxy)-9-((4-methylpyridin-2-yl)methyl)-9H-purin-8-
yl)phenyl)pyrrolidine-3-carboxamide
1-(3-chloro-4-(6-(1-methylcyclopropoxy)-9-((4-methylpyridin-2-yl)methyl)-9H-purin-8-
yl)phenyl)-N-methylazetidine-3-carboxamide
(R)-N-(3-chloro-4-(6-(1-methylcyclopropoxy)-9-((4-methylpyridin-2-yl)methyl)-9H-purin-8-
yl)phenyl)-3-hydroxypyrrolidine-1-carboxamide
3-(3-chloro-4-(6-(1-methylcyclopropoxy)-9-((4-methylpyridin-2-yl)methyl)-9H-purin-8-
yl)phenoxy)-N-methylazetidine-1-carboxamide
(R)-3-(3-chloro-4-(6-(1-methylcyclopropoxy)-9-((4-methylpyridin-2-yl)methyl)-9H-purin-8-
yl)phenoxy)pyrrolidine-1-carboxamide
9-((4-chloro-3-fluoropyridin-2-yl)methyl)-8-(2-chloro-4-(2-(piperazin-1-yl)ethoxy)phenyl)-6-
(1-methylcyclopropoxy)-9H-purine
8-(2-chloro-4-(2-(piperazin-1-yl)ethoxy)phenyl)-9-((4-chloro-5-fluoropyridin-2-yl)methyl)-6-
(1-methylcyclopropoxy)-9H-purine
1-(3-(3-chloro-4-(9-((4-chloropyridin-2-yl)methyl)-6-(1-methylcyclopropoxy)-9H-purin-8-
yl)phenoxy)propyl)azetidin-3-ol
8-(2-bromo-4-(2-(piperazin-1-yl)ethoxy)phenyl)-9-((4-chloropyridin-2-yl)methyl)-6-(1-
methylcyclopropoxy)-9H-purine
9-((4-chloropyridin-2-yl)methyl)-8-(2-fluoro-4-(2-(piperazin-1-yl)ethoxy)phenyl)-6-(1-
methylcyclopropoxy)-9H-purine
8-(2-chloro-4-(2-(piperazin-1-yl)ethoxy)phenyl)-9-((5-fluoropyridin-2-yl)methyl)-6-(1-
methylcyclopropoxy)-9H-purine
8-(2-chloro-4-(2-(piperazin-1-yl)ethoxy)phenyl)-9-((3-fluoropyridin-2-yl)methyl)-6-(1-
methylcyclopropoxy)-9H-purine
3-(3-chloro-4-(9-((4-(difluoromethyl)pyridin-2-yl)methyl)-6-(1-methylcyclopropoxy)-9H-purin-
8-yl)phenoxy)-N,N-dimethylpropan-1-amine
(R)-1-(3-chloro-4-(6-(1-methylcyclopropoxy)-9-((4-methylpyridin-2-yl)methyl)-9H-purin-8-
yl)benzyl)pyrrolidin-3-ol
8-(2-chloro-4-(piperidin-4-yloxy)phenyl)-6-(1-methylcyclopropoxy)-9-((4-methylpyridin-2-
yl)methyl)-9H-purine
1-(3-chloro-4-(6-(1-methylcyclopropoxy)-9-((4-methylpyridin-2-yl)methyl)-9H-purin-8-
yl)phenethyl)azetidin-3-ol
(R)-8-(2-chloro-4-(pyrrolidin-2-ylmethoxy)phenyl)-6-(1-methylcyclopropoxy)-9-((4-
methylpyridin-2-yl)methyl)-9H-purine
2-(3-chloro-4-(6-(1-methylcyclopropoxy)-9-((4-methylpyridin-2-yl)methyl)-9H-purin-8-
yl)phenyl)-N-isopropylacetamide
3-(3-chloro-4-(6-(1-methylcyclopropoxy)-9-((4-methylpyridin-2-yl)methyl)-9H-purin-8-
yl)phenyl)-N,N-dimethylpropanamide
2-((8-(2-chloro-4-((2-methyl-2-azaspiro[3.3]heptan-6-yl)oxy)phenyl)-6-(1-
methylcyclopropoxy)-9H-purin-9-yl)methyl)-5-methylthiazole
1-(2-(2-chloro-3-(6-(1-methylcyclopropoxy)-9-((4-methylpyridin-2-yl)methyl)-9H-purin-8-
yl)phenoxy)ethyl)azetidin-3-ol
(R)-3-chloro-N-(1-hydroxybutan-2-yl)-4-(6-(1-methylcyclopropoxy)-9-((4-methylpyridin-2-
yl)methyl)-9H-purin-8-yl)benzamide
3-chloro-N-(1-hydroxy-2-methylpropan-2-yl)-4-(6-(1-methylcyclopropoxy)-9-((4-
methylpyridin-2-yl)methyl)-9H-purin-8-yl)benzamide
3-chloro-N-(2-hydroxy-2-methylpropyl)-4-(6-(1-methylcyclopropoxy)-9-((4-methylpyridin-2-
yl)methyl)-9H-purin-8-yl)benzamide
(R)-3-chloro-N-(4-hydroxybutan-2-yl)-4-(6-(1-methylcyclopropoxy)-9-((4-methylpyridin-2-
yl)methyl)-9H-purin-8-yl)benzamide
(R)-3-chloro-N-(2-hydroxybutyl)-4-(6-(1-methylcyclopropoxy)-9-((4-methylpyridin-2-
yl)methyl)-9H-purin-8-yl)benzamide
2-(3-chloro-4-(6-(1-methylcyclopropoxy)-9-((4-methylpyridin-2-yl)methyl)-9H-purin-8-
yl)phenoxy)-N-isopropylacetamide
N-(3-(3-chloro-4-(6-(1-methylcyclopropoxy)-9-((4-methylpyridin-2-yl)methyl)-9H-purin-8-
yl)phenoxy)propyl)acetamide
(R)-N-(3-chloro-4-(6-(1-methylcyclopropoxy)-9-((4-methylpyridin-2-yl)methyl)-9H-purin-8-
yl)phenethyl)-2-hydroxypropanamide
2-(3-chloro-4-(6-(1-methylcyclopropoxy)-9-((4-methylpyridin-2-yl)methyl)-9H-purin-8-
yl)phenyl)-N-(2-hydroxyethyl)-N-methylacetamide
2-(3-chloro-4-(6-(1-methylcyclopropoxy)-9-((4-methylpyridin-2-yl)methyl)-9H-purin-8-
yl)phenyl)-N-(3-hydroxypropyl)acetamide
(S)-2-(3-chloro-4-(6-(1-methylcyclopropoxy)-9-((4-methylpyridin-2-yl)methyl)-9H-purin-8-
yl)phenyl)-N-(2-hydroxypropyl)acetamide
(S)-2-(3-chloro-4-(6-(1-methylcyclopropoxy)-9-((4-methylpyridin-2-yl)methyl)-9H-purin-8-
yl)phenoxy)-N-(2-hydroxypropyl)acetamide
2-(3-chloro-4-(6-(1-methylcyclopropoxy)-9-((4-methylpyridin-2-yl)methyl)-9H-purin-8-
yl)phenoxy)-N-(3-hydroxypropyl)acetamide
2-(3-chloro-4-(6-(1-methylcyclopropoxy)-9-((4-methylpyridin-2-yl)methyl)-9H-purin-8-
yl)phenoxy)-N-(2-hydroxyethyl)-N-methylacetamide
(R)-N-(2-(3-chloro-4-(6-(1-methylcyclopropoxy)-9-((4-methylpyridin-2-yl)methyl)-9H-purin-8-
yl)phenoxy)ethyl)-2-hydroxypropanamide
3-(3-chloro-4-(1-((4-chloropyridin-2-yl)methyl)-4-(1-methylcyclopropoxy)-1H-imidazo[4,5-
c]pyridin-2-yl)phenoxy)-N,N-dimethylpropan-1-amine
8-(2-chloro-4-(2-(piperazin-1-yl)ethoxy)phenyl)-9-((2-chloropyridin-4-yl)methyl)-6-(1-
methylcyclopropoxy)-9H-purine
8-(2-chloro-4-(2-(piperazin-1-yl)ethoxy)phenyl)-9-((5-chloropyridin-3-yl)methyl)-6-(1-
methylcyclopropoxy)-9H-purine
4-(2-(3-chloro-4-(9-((4-chloropyridin-2-yl)methyl)-6-(1-methylcyclopropoxy)-9H-purin-8-
yl)phenoxy)ethyl)piperazine-1-sulfonic acid
2-(3-chloro-4-(4-(1-methylcyclopropoxy)-1-((4-methylpyridin-2-yl)methyl)-1H-imidazo[4,5-
c]pyridin-2-yl)phenoxy)-N,N-dimethylethan-1-amine
4-(3-chloro-4-(6-(1-methylcyclopropoxy)-9-((4-methylpyridin-2-yl)methyl)-9H-purin-8-
yl)phenoxy)-2-methylbutan-2-ol
3-(3-chloro-4-(6-(1-methylcyclopropoxy)-9-((4-methylpyridin-2-yl)methyl)-9H-purin-8-
yl)phenoxy)-2,2-dimethylpropan-1-ol
8-(2-chloro-4-(1,4-diazepan-1-yl)phenyl)-6-(1-methylcyclopropoxy)-9-((4-methylpyridin-2-
yl)methyl)-9H-purine
3-(3-chloro-4-(6-(1-methylcyclopropoxy)-9-((4-methylpyridin-2-yl)methyl)-9H-purin-8-
yl)phenoxy)-N,N-dimethylpropan-1-amine
3-(2-chloro-3-(6-(1-methylcyclopropoxy)-9-((4-methylpyridin-2-yl)methyl)-9H-purin-8-
yl)phenoxy)-N,N-dimethylpropan-1-amine
3-(4-(9-((4-chloropyridin-2-yl)methyl)-6-(1-methylcyclopropoxy)-9H-purin-8-yl)-3-
methylphenoxy)-N,N-dimethylpropan-1-amine
2-((8-(2-chloro-4-(3-(pyrrolidin-1-yl)propoxy)phenyl)-6-(1-methylcyclopropoxy)-9H-purin-9-
yl)methyl)-5-methylthiazole
2-((8-(2-chloro-4-((1-methylazepan-4-yl)oxy)phenyl)-6-(1-methylcyclopropoxy)-9H-purin-9-
yl)methyl)-5-methylthiazole (isomer 1)
2-((8-(2-chloro-4-((1-methylazepan-4-yl)oxy)phenyl)-6-(1-methylcyclopropoxy)-9H-purin-9-
yl)methyl)-5-methylthiazole (isomer 2)
(R)-1-(3-(3-chloro-4-(6-(1-methylcyclopropoxy)-9-((5-methylthiazol-2-yl)methyl)-9H-purin-8-
yl)phenoxy)propyl)pyrrolidin-3-ol
(R)-1-(3-(2-chloro-3-(6-(1-methylcyclopropoxy)-9-((5-methylthiazol-2-yl)methyl)-9H-purin-8-
yl)phenoxy)propyl)pyrrolidin-3-ol
(S)-2-((8-(2-chloro-4-(2-(2-methylpiperazin-1-yl)ethoxy)phenyl)-6-(1-methylcyclopropoxy)-
9H-purin-9-yl)methyl)-5-methylthiazole
3-((3-chloro-4-(9-(3-chlorobenzyl)-6-(1-methylcyclopropoxy)-9H-purin-8-yl)phenoxy)methyl)-
1-methyl-1H-pyrazole-5-carboxylic acid
4-(3-chloro-4-(9-(2-fluoro-5-(trifluoromethyl)benzyl)-6-(1-methylcyclopropoxy)-9H-purin-8-
yl)phenoxy)-2-methylbutanoic acid (racemic)
4-(3-chloro-4-(6-(1-methylcyclopropoxy)-9-(3-(trifluoromethyl)benzyl)-9H-purin-8-
yl)phenoxy)-2-methylbutanoic acid (Isomer 1)
4-(3-chloro-4-(6-(1-methylcyclopropoxy)-9-(3-(trifluoromethyl)benzyl)-9H-purin-8-
yl)phenoxy)-2-methylbutanoic acid (Isomer 2)
4-(3-chloro-4-(9-(3-cyanobenzyl)-6-(1-methylcyclopropoxy)-9H-purin-8-yl)phenoxy)-2-
methylbutanoic acid (racemic)
4-(3-chloro-4-(9-(2-cyanobenzyl)-6-(1-methylcyclopropoxy)-9H-purin-8-yl)phenoxy)-2-
methylbutanoic acid (racemic)
(R)-4-(3-chloro-4-(9-(3-cyanobenzyl)-6-(1-methylcyclopropoxy)-9H-purin-8-yl)phenoxy)-2-
methylbutanoic acid
(R)-4-(3-chloro-4-(9-(2-cyanobenzyl)-6-(1-methylcyclopropoxy)-9H-purin-8-yl)phenoxy)-2-
methylbutanoic acid
(S)-4-(3-chloro-4-(9-(2-cyanobenzyl)-6-(1-methylcyclopropoxy)-9H-purin-8-yl)phenoxy)-2-
methylbutanoic acid
(S)-1-(3-chloro-4-(6-(1-methylcyclopropoxy)-9-((4-methylpyridin-2-yl)methyl)-9H-purin-8-
yl)benzoyl)pyrrolidine-2-carbonitrile
(R)-1-(3-chloro-4-(6-(1-methylcyclopropoxy)-9-((4-methylpyridin-2-yl)methyl)-9H-purin-8-
yl)benzoyl)pyrrolidine-3-carbonitrile
3-(3-chloro-4-(1-(3-chlorobenzyl)-4-(1-methylcyclobutoxy)-1H-benzo[d]imidazol-2-
yl)phenoxy)propanoic acid
1-(2-(3-chloro-4-(9-(3-chlorobenzyl)-6-(1-methylcyclopropoxy)-9H-purin-8-
yl)phenoxy)ethyl)cyclopropane-1-carboxylic acid
(1s,3s)-3-((3-chloro-4-(9-(3-chlorobenzyl)-6-(1-methylcyclopropoxy)-9H-purin-8-
yl)phenoxy)methyl)cyclobutane-1-carboxylic acid
(1r,3r)-3-((3-chloro-4-(9-(3-chlorobenzyl)-6-(1-methylcyclopropoxy)-9H-purin-8-
yl)phenoxy)methyl)cyclobutane-1-carboxylic acid
4-(3-chloro-4-(9-(3-(difluoromethyl)benzyl)-6-(1-methylcyclopropoxy)-9H-purin-8-
yl)phenoxy)-2-methylbutanoic acid (Isomer 1)
4-(3-chloro-4-(9-(3-(difluoromethyl)benzyl)-6-(1-methylcyclopropoxy)-9H-purin-8-
yl)phenoxy)-2-methylbutanoic acid (Isomer 2)
4-(3-chloro-4-(9-(2-(difluoromethyl)benzyl)-6-(1-methylcyclopropoxy)-9H-purin-8-
yl)phenoxy)-2-methylbutanoic acid (Isomer 1)
4-(3-chloro-4-(9-(2-(difluoromethyl)benzyl)-6-(1-methylcyclopropoxy)-9H-purin-8-
yl)phenoxy)-2-methylbutanoic acid (Isomer 2)
((1S,4S)-2-oxa-5-azabicyclo[2.2.1]heptan-5-yl)(3-chloro-4-(6-(1-methylcyclopropoxy)-9-((4-
methylpyridin-2-yl)methyl)-9H-purin-8-yl)phenyl)methanone
4-(3-chloro-4-(9-(2-fluoro-5-methylbenzyl)-6-(1-methylcyclopropoxy)-9H-purin-8-
yl)phenoxy)-2-methylbutanoic acid (racemic)
(S)-(3-chloro-4-(6-(1-methylcyclopropoxy)-9-((4-methylpyridin-2-yl)methyl)-9H-purin-8-
yl)phenyl)(3-fluoropiperidin-1-yl)methanone
(3-chloro-4-(6-(1-methylcyclopropoxy)-9-((4-methylpyridin-2-yl)methyl)-9H-purin-8-
yl)phenyl)(4-fluoropiperidin-1-yl)methanone
(S)-2-(3-chloro-4-(6-(1-methylcyclopropoxy)-9-((4-methylpyridin-2-yl)methyl)-9H-purin-8-
yl)phenoxy)-1-(3-fluoropyrrolidin-1-yl)ethan-1-one
2-(3-chloro-4-(6-(1-methylcyclopropoxy)-9-((4-methylpyridin-2-yl)methyl)-9H-purin-8-
yl)phenoxy)-N-(3-fluorocyclobutyl)acetamide
2-(3-chloro-4-(6-(1-methylcyclopropoxy)-9-((4-methylpyridin-2-yl)methyl)-9H-purin-8-
yl)phenyl)-1-((3R,4R)-3-fluoro-4-hydroxypyrrolidin-1-yl)ethan-1-one
2-(3-chloro-4-(6-(1-methylcyclopropoxy)-9-((4-methylpyridin-2-yl)methyl)-9H-purin-8-
yl)phenoxy)-1-((3R,4R)-3-fluoro-4-hydroxypyrrolidin-1-yl)ethan-1-one
9-benzyl-8-(2-chloro-4-(2-(piperazin-1-yl)ethoxy)phenyl)-6-((3-(trifluoromethyl)oxetan-3-
yl)oxy)-9H-purine
1-(3-(3-chloro-4-(6-(1-methylcyclopropoxy)-9-((4-(trifluoromethyl)pyridin-2-yl)methyl)-9H-
purin-8-yl)phenoxy)propyl)azetidin-3-ol
8-(4-((2-oxaspiro[3.3]heptan-6-yl)oxy)-2-chlorophenyl)-6-(1-methylcyclopropoxy)-9-((4-
methylpyridin-2-yl)methyl)-9H-purine
(3,6-diazabicyclo[3.1.1]heptan-3-yl)(3-chloro-4-(6-(1-methylcyclopropoxy)-9-((4-
methylpyridin-2-yl)methyl)-9H-purin-8-yl)phenyl)methanone
(3-chloro-4-(6-(1-methylcyclopropoxy)-9-((4-methylpyridin-2-yl)methyl)-9H-purin-8-
yl)phenyl)(2,6-diazaspiro[3.3]heptan-2-yl)methanone
(3-chloro-4-(6-(1-methylcyclopropoxy)-9-((4-methylpyridin-2-yl)methyl)-9H-purin-8-
yl)phenyl)(1,6-diazaspiro[3.3]heptan-6-yl)methanone
2-(3-chloro-4-(6-(1-methylcyclopropoxy)-9-((4-methylpyridin-2-yl)methyl)-9H-purin-8-
yl)phenoxy)-N-(2-cyanoethyl)-N-methylacetamide
4-(2-(3-chloro-4-(6-(1-methylcyclopropoxy)-9-((4-methylpyridin-2-yl)methyl)-9H-purin-8-
yl)phenyl)acetyl)piperazin-2-one
4-(2-(3-chloro-4-(6-(1-methylcyclopropoxy)-9-((4-methylpyridin-2-yl)methyl)-9H-purin-8-
yl)phenoxy)acetyl)piperazin-2-one
3-((3-chloro-4-(6-(1-methylcyclopropoxy)-9-((4-methylpyridin-2-yl)methyl)-9H-purin-8-
yl)phenoxy)methyl)-5,6,7,8-tetrahydro-[1,2,4]triazolo[4,3-a]pyrazine
5-(3-chloro-4-(9-(3-chlorobenzyl)-6-(1-methylcyclopropoxy)-9H-purin-8-yl)phenyl)-2-
methylpentanoic acid (racemic)
4-(3-chloro-4-(9-(3-chlorobenzyl)-6-(1-methylcyclopropoxy)-9H-purin-8-yl)phenoxy)-2,2-
dimethylbutanoic acid
4-(3-chloro-4-(9-(3-chlorobenzyl)-6-(1-methylcyclopropoxy)-9H-purin-8-yl)phenoxy)-3,3-
dimethylbutanoic acid
6-(3-chloro-4-(9-(3-chlorobenzyl)-6-(1-methylcyclopropoxy)-9H-purin-8-yl)phenoxy)hexanoic
acid
5-(3-chloro-4-(9-(3-chlorobenzyl)-6-(1-methylcyclopropoxy)-9H-purin-8-yl)phenoxy)-2-
methylpentanoic acid (isomer 2)
5-(3-chloro-4-(9-(3-chlorobenzyl)-6-(1-methylcyclopropoxy)-9H-purin-8-yl)phenoxy)-2-
methylpentanoic acid (isomer 1)
6-(2-chloro-3-(9-(3-chlorobenzyl)-6-(1-methylcyclopropoxy)-9H-purin-8-yl)phenoxy)hexanoic
acid
4-(3-chloro-4-(9-(3-chlorobenzyl)-6-(1-methylcyclopropoxy)-9H-purin-8-yl)phenoxy)-2-
ethylbutanoic acid (racemic)
4-(2-chloro-3-(9-(3-chlorobenzyl)-6-(1-methylcyclopropoxy)-9H-purin-8-yl)phenoxy)-2,2-
dimethylbutanoic acid
5-(2-chloro-3-(9-(3-chlorobenzyl)-6-(1-methylcyclopropoxy)-9H-purin-8-yl)phenoxy)-2-
methylpentanoic acid (Isomer 1)
5-(2-chloro-3-(9-(3-chlorobenzyl)-6-(1-methylcyclopropoxy)-9H-purin-8-yl)phenoxy)-2-
methylpentanoic acid (Isomer 2)
2-(4-(3-chloro-4-(9-(3-chlorobenzyl)-6-(1-methylcyclopropoxy)-9H-purin-8-
yl)phenoxy)butoxy)acetic acid
4-(3-chloro-4-(9-(3-chloro-5-methoxybenzyl)-6-(1-methylcyclopropoxy)-9H-purin-8-
yl)phenoxy)-2-methylbutanoic acid (Isomer 1)
4-(3-chloro-4-(9-(3-chloro-5-methoxybenzyl)-6-(1-methylcyclopropoxy)-9H-purin-8-
yl)phenoxy)-2-methylbutanoic acid (Isomer 2)
2-(4-(2-chloro-3-(9-(3-chlorobenzyl)-6-(1-methylcyclopropoxy)-9H-purin-8-
yl)phenoxy)butoxy)acetic acid
3-(3-(3-chloro-4-(9-(3-chlorobenzyl)-6-(1-methylcyclopropoxy)-9H-purin-8-
yl)phenoxy)propoxy)propanoic acid
2-(2-(3-chloro-4-(9-(3-chlorobenzyl)-6-(1-methylcyclopropoxy)-9H-purin-8-
yl)phenoxy)ethoxy)-2-methylpropanoic acid
2-(2-(2-(3-chloro-4-(9-(3-chlorobenzyl)-6-(1-methylcyclopropoxy)-9H-purin-8-
yl)phenoxy)ethoxy)ethoxy)acetic acid
1-(3-(3-chloro-4-(9-((4-(difluoromethyl)pyridin-2-yl)methyl)-6-(1-methylcyclopropoxy)-9H-
purin-8-yl)phenoxy)propyl)azetidin-3-ol
9-((4-chloropyridin-2-yl)methyl)-6-(1-methylcyclopropoxy)-8-(4-(2-(piperazin-1-yl)ethoxy)-2-
(trifluoromethyl)phenyl)-9H-purine
4-(3-chloro-4-(9-(3-methoxybenzyl)-6-(1-methylcyclopropoxy)-9H-purin-8-yl)phenoxy)-2-
methylbutanoic acid (racemic)
4-(3-chloro-4-(9-(3-methoxybenzyl)-6-(1-methylcyclopropoxy)-9H-purin-8-yl)phenoxy)-2-
methylbutanoic acid (Isomer 1)
4-(3-chloro-4-(9-(3-methoxybenzyl)-6-(1-methylcyclopropoxy)-9H-purin-8-yl)phenoxy)-2-
methylbutanoic acid (Isomer 2)
8-(4-((2-azaspiro[3.3]heptan-6-yl)oxy)-2-chlorophenyl)-6-(1-methylcyclopropoxy)-9-((4-
methylpyridin-2-yl)methyl)-9H-purine
(R)-2-(3-chloro-4-(6-(1-methylcyclopropoxy)-9-((4-methylpyridin-2-yl)methyl)-9H-purin-8-
yl)phenyl)-1-(3-hydroxypyrrolidin-1-yl)ethan-1-one
(R)-(3-chloro-4-(6-(1-methylcyclopropoxy)-9-((4-methylpyridin-2-yl)methyl)-9H-purin-8-
yl)phenyl)(2-(hydroxymethyl)pyrrolidin-1-yl)methanone
(3-chloro-4-(6-(1-methylcyclopropoxy)-9-((4-methylpyridin-2-yl)methyl)-9H-purin-8-
yl)phenyl)(4-hydroxypiperidin-1-yl)methanone
(S)-(3-chloro-4-(6-(1-methylcyclopropoxy)-9-((4-methylpyridin-2-yl)methyl)-9H-purin-8-
yl)phenyl)(3-hydroxypiperidin-1-yl)methanone
3-chloro-N-((1-hydroxycyclopropyl)methyl)-N-methyl-4-(6-(1-methylcyclopropoxy)-9-((4-
methylpyridin-2-yl)methyl)-9H-purin-8-yl)benzamide
(R)-(3-chloro-4-(6-(1-methylcyclopropoxy)-9-((4-methylpyridin-2-yl)methyl)-9H-purin-8-
yl)phenyl)(3-hydroxy-3-methylpyrrolidin-1-yl)methanone
(R)-(3-chloro-4-(6-(1-methylcyclopropoxy)-9-((4-methylpyridin-2-yl)methyl)-9H-purin-8-
yl)phenyl)(3-(hydroxymethyl)pyrrolidin-1-yl)methanone
(S)-(3-chloro-4-(6-(1-methylcyclopropoxy)-9-((4-methylpyridin-2-yl)methyl)-9H-purin-8-
yl)phenyl)(3-methoxypyrrolidin-1-yl)methanone
(S)-(3-chloro-4-(6-(1-methylcyclopropoxy)-9-((4-methylpyridin-2-yl)methyl)-9H-purin-8-
yl)phenyl)(2-(hydroxymethyl)pyrrolidin-1-yl)methanone
2-(3-chloro-4-(6-(1-methylcyclopropoxy)-9-((4-methylpyridin-2-yl)methyl)-9H-purin-8-
yl)phenoxy)-1-(pyrrolidin-1-yl)ethan-1-one
3-chloro-N-((1s,3s)-3-hydroxycyclobutyl)-N-methyl-4-(6-(1-methylcyclopropoxy)-9-((4-
methylpyridin-2-yl)methyl)-9H-purin-8-yl)benzamide
1-(2-(3-chloro-4-(6-(1-methylcyclopropoxy)-9-((4-methylpyridin-2-yl)methyl)-9H-purin-8-
yl)phenoxy)ethyl)pyrrolidin-2-one
2-(3-chloro-4-(6-(1-methylcyclopropoxy)-9-((4-methylpyridin-2-yl)methyl)-9H-purin-8-
yl)phenyl)-1-(3-hydroxy-3-methylazetidin-1-yl)ethan-1-one
1-(3-(3-chloro-4-(6-(1-methylcyclopropoxy)-9-((4-methylpyridin-2-yl)methyl)-9H-purin-8-
yl)phenoxy)azetidin-1-yl)propan-1-one
(R)-1-(3-(3-chloro-4-(6-(1-methylcyclopropoxy)-9-((4-methylpyridin-2-yl)methyl)-9H-purin-8-
yl)phenoxy)pyrrolidin-1-yl)ethan-1-one
(R)-2-(3-chloro-4-(6-(1-methylcyclopropoxy)-9-((4-methylpyridin-2-yl)methyl)-9H-purin-8-
yl)phenoxy)-1-(3-hydroxypyrrolidin-1-yl)ethan-1-one
(R)-1-(2-(3-chloro-4-(6-(1-methylcyclopropoxy)-9-((4-methylpyridin-2-yl)methyl)-9H-purin-8-
yl)phenoxy)ethyl)-3-hydroxypyrrolidin-2-one
2-(3-chloro-4-(6-(1-methylcyclopropoxy)-9-((4-methylpyridin-2-yl)methyl)-9H-purin-8-
yl)phenoxy)-1-morpholinoethan-1-one
2-(3-chloro-4-(6-(1-methylcyclopropoxy)-9-((4-methylpyridin-2-yl)methyl)-9H-purin-8-
yl)phenoxy)-N-(3-methyloxetan-3-yl)acetamide
2-(3-chloro-4-(6-(1-methylcyclopropoxy)-9-((4-methylpyridin-2-yl)methyl)-9H-purin-8-
yl)phenoxy)-1-(3-hydroxy-3-methylazetidin-1-yl)ethan-1-one
(R)-2-(3-chloro-4-(6-(1-methylcyclopropoxy)-9-((4-methylpyridin-2-yl)methyl)-9H-purin-8-
yl)phenoxy)-N-(tetrahydrofuran-3-yl)acetamide
2-(3-chloro-4-(6-(1-methylcyclopropoxy)-9-((4-methylpyridin-2-yl)methyl)-9H-purin-8-
yl)phenoxy)-N-(3-hydroxycyclobutyl)acetamide
2-(3-chloro-4-(6-(1-methylcyclopropoxy)-9-((4-methylpyridin-2-yl)methyl)-9H-purin-8-
yl)phenoxy)-N-methyl-N-(oxetan-3-yl)acetamide
4-(2-(3-chloro-4-(6-(1-methylcyclopropoxy)-9-((4-methylpyridin-2-yl)methyl)-9H-purin-8-
yl)phenoxy)ethyl)morpholin-3-one
(R)-1-(3-(3-chloro-4-(6-(1-methylcyclopropoxy)-9-((4-methylpyridin-2-yl)methyl)-9H-purin-8-
yl)phenoxy)azetidin-1-yl)-2-hydroxypropan-1-one
(S)-1-(3-(3-chloro-4-(6-(1-methylcyclopropoxy)-9-((4-methylpyridin-2-yl)methyl)-9H-purin-8-
yl)phenoxy)pyrrolidin-1-yl)-2-hydroxyethan-1-one
6-((8-(2-chloro-4-(2-(piperazin-1-yl)ethoxy)phenyl)-6-(1-methylcyclopropoxy)-9H-purin-9-
yl)methyl)nicotinonitrile
8-(4-(2-((1R,4R)-2,5-diazabicyclo[2.2.1]heptan-2-yl)ethoxy)-2-chlorophenyl)-9-((4-
chloropyridin-2-yl)methyl)-6-(1-methylcyclopropoxy)-9H-purine
8-(4-(2-(1,6-diazaspiro[3.3]heptan-6-yl)ethoxy)-2-chlorophenyl)-9-((4-chloropyridin-2-
yl)methyl)-6-(1-methylcyclopropoxy)-9H-purine
8-(4-(2-((1S,4S)-2,5-diazabicyclo[2.2.1]heptan-2-yl)ethoxy)-2-chlorophenyl)-9-((4-
chloropyridin-2-yl)methyl)-6-(1-methylcyclopropoxy)-9H-purine
8-(2-chloro-4-(2-(piperazin-1-yl)ethoxy)phenyl)-9-((4-(difluoromethyl)pyridin-2-yl)methyl)-6-
(1-methylcyclopropoxy)-9H-purine
8-(4-((1,6-diazaspiro[3.3]heptan-6-yl)methyl)-2-chlorophenyl)-6-(1-methylcyclopropoxy)-9-
((4-methylpyridin-2-yl)methyl)-9H-purine
8-(2-chloro-4-(2,6-diazaspiro[3.4]octan-6-yl)phenyl)-6-(1-methylcyclopropoxy)-9-((4-
methylpyridin-2-yl)methyl)-9H-purine
8-(4-(2,6-diazabicyclo[3.2.1]octan-6-yl)-2-chlorophenyl)-6-(1-methylcyclopropoxy)-9-((4-
methylpyridin-2-yl)methyl)-9H-purine (racemic)
8-(2-chloro-4-(hexahydropyrrolo[3,2-b]pyrrol-1(2H)-yl)phenyl)-6-(1-methylcyclopropoxy)-9-
((4-methylpyridin-2-yl)methyl)-9H-purine (unknown absolute stereochemistry)
8-(4-(1,4-diazabicyclo[3.2.1]octan-4-yl)-2-chlorophenyl)-6-(1-methylcyclopropoxy)-9-((4-
methylpyridin-2-yl)methyl)-9H-purine (racemic)
8-(2-chloro-4-((3aR,6aR)-hexahydropyrrolo[3,4-b]pyrrol-5(1H)-yl)phenyl)-6-(1-
methylcyclopropoxy)-9-((4-methylpyridin-2-yl)methyl)-9H-purine
2-(3-chloro-4-(6-(1-methylcyclopropoxy)-9-((4-methylpyridin-2-yl)methyl)-9H-purin-8-
yl)phenyl)-1-(piperazin-1-yl)ethan-1-one
(3-chloro-4-(6-(1-methylcyclopropoxy)-9-((4-methylpyridin-2-yl)methyl)-9H-purin-8-
yl)phenyl)(1,4-diazepan-1-yl)methanone
(S)-1-(3-chloro-4-(6-(1-methylcyclopropoxy)-9-((4-methylpyridin-2-yl)methyl)-9H-purin-8-
yl)phenyl)-N-methylpyrrolidine-3-carboxamide
1-(2-(3-chloro-4-(6-(1-methylcyclopropoxy)-9-((4-methylpyridin-2-yl)methyl)-9H-purin-8-
yl)phenoxy)ethyl)piperazin-2-one
4-(2-(3-chloro-4-(6-(1-methylcyclopropoxy)-9-((4-methylpyridin-2-yl)methyl)-9H-purin-8-
yl)phenoxy)ethyl)piperazin-2-one
3-(3-chloro-4-(6-(1-methylcyclopropoxy)-9-((4-methylpyridin-2-yl)methyl)-9H-purin-8-
yl)phenoxy)-N,N-dimethylazetidine-1-carboxamide
(R)-3-(3-chloro-4-(6-(1-methylcyclopropoxy)-9-((4-methylpyridin-2-yl)methyl)-9H-purin-8-
yl)phenoxy)-N-methylpyrrolidine-1-carboxamide
(R)-1-(3-(3-chloro-4-(9-((4-chloropyridin-2-yl)methyl)-6-(1-methylcyclopropoxy)-9H-purin-8-
yl)phenoxy)propyl)pyrrolidin-3-ol
(S)-1-(3-(3-chloro-4-(9-((4-chloropyridin-2-yl)methyl)-6-(1-methylcyclopropoxy)-9H-purin-8-
yl)phenoxy)propyl)pyrrolidin-3-ol
8-(2-chloro-4-(2-(piperazin-1-yl)ethoxy)phenyl)-9-((3-fluoro-4-methylpyridin-2-yl)methyl)-6-
(1-methylcyclopropoxy)-9H-purine
8-(2-chloro-4-(2-(pyrrolidin-1-yl)ethyl)phenyl)-6-(1-methylcyclopropoxy)-9-((4-methylpyridin-
2-yl)methyl)-9H-purine
9-benzyl-8-(2-chloro-4-(2-(piperazin-1-yl)ethoxy)phenyl)-6-cyclobutoxy-9H-purine
8-(2-chloro-4-(2-(pyrrolidin-1-yl)ethoxy)phenyl)-6-(1-methylcyclopropoxy)-9-((4-
methylpyridin-2-yl)methyl)-9H-purine
1-(3-chloro-4-(6-(1-methylcyclopropoxy)-9-((4-methylpyridin-2-yl)methyl)-9H-purin-8-
yl)benzyl)piperidin-4-ol
8-(4-(azepan-4-yloxy)-2-chlorophenyl)-6-(1-methylcyclopropoxy)-9-((4-methylpyridin-2-
yl)methyl)-9H-purine (isomer 2)
8-(4-(azepan-4-yloxy)-2-chlorophenyl)-6-(1-methylcyclopropoxy)-9-((4-methylpyridin-2-
yl)methyl)-9H-purine (isomer 1)
(R)-1-(3-chloro-4-(6-(1-methylcyclopropoxy)-9-((4-methylpyridin-2-yl)methyl)-9H-purin-8-
yl)phenethyl)pyrrolidin-3-ol
(R)-1-(2-(3-chloro-4-(6-(1-methylcyclopropoxy)-9-((4-methylpyridin-2-yl)methyl)-9H-purin-8-
yl)phenoxy)ethyl)pyrrolidin-3-ol
1-(3-(3-chloro-4-(6-(1-methylcyclopropoxy)-9-((4-methylpyridin-2-yl)methyl)-9H-purin-8-
yl)phenoxy)propyl)azetidin-3-ol
1-(3-(2-chloro-3-(6-(1-methylcyclopropoxy)-9-((4-methylpyridin-2-yl)methyl)-9H-purin-8-
yl)phenoxy)propyl)azetidin-3-ol
2-(3-chloro-4-(6-(1-methylcyclopropoxy)-9-((4-methylpyridin-2-yl)methyl)-9H-purin-8-
yl)phenyl)-N-(2-hydroxy-2-methylpropyl)acetamide
(R)-2-(3-chloro-4-(6-(1-methylcyclopropoxy)-9-((4-methylpyridin-2-yl)methyl)-9H-purin-8-
yl)phenoxy)-N-(4-hydroxybutan-2-yl)acetamide
2-(3-chloro-4-(6-(1-methylcyclopropoxy)-9-((4-methylpyridin-2-yl)methyl)-9H-purin-8-
yl)phenoxy)-N-(2-hydroxy-2-methylpropyl)acetamide
(R)-2-(3-chloro-4-(6-(1-methylcyclopropoxy)-9-((4-methylpyridin-2-yl)methyl)-9H-purin-8-
yl)phenoxy)-N-(1-hydroxybutan-2-yl)acetamide
2-(3-chloro-4-(6-(1-methylcyclopropoxy)-9-((4-methylpyridin-2-yl)methyl)-9H-purin-8-
yl)phenoxy)-N-(1-hydroxy-2-methylpropan-2-yl)acetamide
(R)-2-(3-chloro-4-(6-(1-methylcyclopropoxy)-9-((4-methylpyridin-2-yl)methyl)-9H-purin-8-
yl)phenoxy)-N-(2-hydroxybutyl)acetamide
(R)-8-(2-chloro-4-(2-(2-methylpiperazin-1-yl)ethoxy)phenyl)-9-((4-chloropyridin-2-yl)methyl)-
6-(1-methylcyclopropoxy)-9H-purine
(S)-8-(2-chloro-4-(2-(2-methylpiperazin-1-yl)ethoxy)phenyl)-9-((4-chloropyridin-2-yl)methyl)-
6-(1-methylcyclopropoxy)-9H-purine
8-(2-chloro-4-(2-(piperazin-1-yl)ethoxy)phenyl)-9-((6-chloro-4-methylpyridin-2-yl)methyl)-6-
(1-methylcyclopropoxy)-9H-purine
9-((4-chloro-3-methylpyridin-2-yl)methyl)-8-(2-chloro-4-(2-(piperazin-1-yl)ethoxy)phenyl)-6-
(1-methylcyclopropoxy)-9H-purine
8-(2-chloro-4-(2-(piperazin-1-yl)ethoxy)phenyl)-9-((4-chloro-5-methylpyridin-2-yl)methyl)-6-
(1-methylcyclopropoxy)-9H-purine
8-(2-chloro-4-(2-(piperazin-1-yl)ethoxy)phenyl)-9-((4-chloro-6-methylpyridin-2-yl)methyl)-6-
(1-methylcyclopropoxy)-9H-purine
8-(2-chloro-4-(2-(piperazin-1-yl)ethyl)phenyl)-6-(1-methylcyclopropoxy)-9-((4-methylpyridin-
2-yl)methyl)-9H-purine
9-((4-chloropyridin-2-yl)methyl)-8-(2-methyl-4-(2-(piperazin-1-yl)ethoxy)phenyl)-6-(1-
methylcyclopropoxy)-9H-purine
8-(2-chloro-4-(2-(piperazin-1-yl)ethoxy)phenyl)-6-(1-methylcyclopropoxy)-9-((5-
methylpyridin-3-yl)methyl)-9H-purine
8-(2-chloro-4-(2-(piperazin-1-yl)ethoxy)phenyl)-6-(1-methylcyclopropoxy)-9-((2-
methylpyridin-4-yl)methyl)-9H-purine
8-(2-chloro-4-(2-(piperazin-1-yl)ethoxy)phenyl)-9-((5-methoxypyridin-2-yl)methyl)-6-(1-
methylcyclopropoxy)-9H-purine
4-(4-(9-(3,5-bis(trifluoromethyl)benzyl)-6-(1-methylcyclopropoxy)-9H-purin-8-yl)-3-
chlorophenoxy)-2-methylbutanoic acid (racemic)
4-(3-chloro-4-(9-(2-methyl-3-(trifluoromethyl)benzyl)-6-(1-methylcyclopropoxy)-9H-purin-8-
yl)phenoxy)-2-methylbutanoic acid (isomer 1)
4-(3-chloro-4-(9-(2-methyl-3-(trifluoromethyl)benzyl)-6-(1-methylcyclopropoxy)-9H-purin-8-
yl)phenoxy)-2-methylbutanoic acid (isomer 2)
8-(2-chloro-4-((5,6,7,8-tetrahydroimidazo[1,2-a]pyridin-6-yl)oxy)phenyl)-6-(1-
methylcyclopropoxy)-9-((4-methylpyridin-2-yl)methyl)-9H-purine (Isomer 2)
8-(2-chloro-4-((5,6,7,8-tetrahydroimidazo[1,2-a]pyridin-6-yl)oxy)phenyl)-6-(1-
methylcyclopropoxy)-9-((4-methylpyridin-2-yl)methyl)-9H-purine (Isomer 1)
(R)-1-(2-(3-chloro-4-(6-(1-methylcyclopropoxy)-9-((4-methylpyridin-2-yl)methyl)-9H-purin-8-
yl)phenoxy)acetyl)pyrrolidine-3-carbonitrile
(S)-1-(2-(3-chloro-4-(6-(1-methylcyclopropoxy)-9-((4-methylpyridin-2-yl)methyl)-9H-purin-8-
yl)phenoxy)acetyl)pyrrolidine-2-carbonitrile
(1r,4r)-4-(3-chloro-4-(9-(3-chlorobenzyl)-6-(1-methylcyclopropoxy)-9H-purin-8-
yl)phenoxy)cyclohexane-1-carboxylic acid
(1s,4s)-4-(3-chloro-4-(9-(3-chlorobenzyl)-6-(1-methylcyclopropoxy)-9H-purin-8-
yl)phenoxy)cyclohexane-1-carboxylic acid
(1r,4r)-4-(2-chloro-3-(9-(3-chlorobenzyl)-6-(1-methylcyclopropoxy)-9H-purin-8-
yl)phenoxy)cyclohexane-1-carboxylic acid
(1s,4s)-4-(2-chloro-3-(9-(3-chlorobenzyl)-6-(1-methylcyclopropoxy)-9H-purin-8-
yl)phenoxy)cyclohexane-1-carboxylic acid
4-(3-chloro-4-(9-(3-(1,1-difluoroethyl)benzyl)-6-(1-methylcyclopropoxy)-9H-purin-8-
yl)phenoxy)-2-methylbutanoic acid (racemic)
4-(3-chloro-4-(9-(3-(1,1-difluoroethyl)benzyl)-6-(1-methylcyclopropoxy)-9H-purin-8-
yl)phenoxy)-2-methylbutanoic acid (Isomer 1)
4-(3-chloro-4-(9-(3-(1,1-difluoroethyl)benzyl)-6-(1-methylcyclopropoxy)-9H-purin-8-
yl)phenoxy)-2-methylbutanoic acid (Isomer 2)
rel-1-((1R,5S)-6-oxa-3-azabicyclo[3.1.1]heptan-3-yl)-2-(3-chloro-4-(6-(1-
methylcyclopropoxy)-9-((4-methylpyridin-2-yl)methyl)-9H-purin-8-yl)phenyl)ethan-1-one
(racemic)
1-((1S,4S)-2-oxa-5-azabicyclo[2.2.1]heptan-5-yl)-2-(3-chloro-4-(6-(1-methylcyclopropoxy)-9-
((4-methylpyridin-2-yl)methyl)-9H-purin-8-yl)phenoxy)ethan-1-one
rel-1-((1R,5S)-6-oxa-3-azabicyclo[3.1.1]heptan-3-yl)-2-(3-chloro-4-(6-(1-
methylcyclopropoxy)-9-((4-methylpyridin-2-yl)methyl)-9H-purin-8-yl)phenoxy)ethan-1-one
(racemic)
(2-(3-chloro-4-(9-(3-chlorobenzyl)-6-(1-methylcyclopropoxy)-9H-purin-8-yl)phenoxy)ethyl)-L-
proline
(S)-2-(3-chloro-4-(6-(1-methylcyclopropoxy)-9-((4-methylpyridin-2-yl)methyl)-9H-purin-8-
yl)phenoxy)-1-(3-fluoropiperidin-1-yl)ethan-1-one
2-(3-chloro-4-(6-(1-methylcyclopropoxy)-9-((4-methylpyridin-2-yl)methyl)-9H-purin-8-
yl)phenoxy)-1-(4-fluoropiperidin-1-yl)ethan-1-one
(R)-1-(3-(3-chloro-4-(6-(1-methylcyclopropoxy)-9-((4-(trifluoromethyl)pyridin-2-yl)methyl)-
9H-purin-8-yl)phenoxy)propyl)pyrrolidin-3-ol
1-(3,6-diazabicyclo[3.1.1]heptan-3-yl)-2-(3-chloro-4-(6-(1-methylcyclopropoxy)-9-((4-
methylpyridin-2-yl)methyl)-9H-purin-8-yl)phenyl)ethan-1-one
2-(3-chloro-4-(6-(1-methylcyclopropoxy)-9-((4-methylpyridin-2-yl)methyl)-9H-purin-8-
yl)phenyl)-1-(1,6-diazaspiro[3.3]heptan-6-yl)ethan-1-one
1-(3,6-diazabicyclo[3.1.1]heptan-6-yl)-2-(3-chloro-4-(6-(1-methylcyclopropoxy)-9-((4-
methylpyridin-2-yl)methyl)-9H-purin-8-yl)phenyl)ethan-1-one
2-(3-chloro-4-(6-(1-methylcyclopropoxy)-9-((4-methylpyridin-2-yl)methyl)-9H-purin-8-
yl)phenyl)-1-(2,6-diazaspiro[3.3]heptan-2-yl)ethan-1-one
1-((1S,4S)-2,5-diazabicyclo[2.2.1]heptan-2-yl)-2-(3-chloro-4-(6-(1-methylcyclopropoxy)-9-
((4-methylpyridin-2-yl)methyl)-9H-purin-8-yl)phenyl)ethan-1-one
7-(2-(3-chloro-4-(6-(1-methylcyclopropoxy)-9-((4-methylpyridin-2-yl)methyl)-9H-purin-8-
yl)phenoxy)ethyl)-5,6,7,8-tetrahydro-[1,2,4]triazolo[4,3-a]pyrazine
7-(3-chloro-4-(9-(3-chlorobenzyl)-6-(1-methylcyclopropoxy)-9H-purin-8-yl)phenoxy)heptanoic
acid
5-(3-chloro-4-(9-(3-chlorobenzyl)-6-(1-methylcyclopropoxy)-9H-purin-8-yl)phenoxy)-3,3-
dimethylpentanoic acid
5-(3-chloro-4-(9-(3-chlorobenzyl)-6-(1-methylcyclopropoxy)-9H-purin-8-yl)phenoxy)-2,2-
dimethylpentanoic acid
7-(2-chloro-3-(9-(3-chlorobenzyl)-6-(1-methylcyclopropoxy)-9H-purin-8-yl)phenoxy)heptanoic
acid
5-(2-chloro-3-(9-(3-chlorobenzyl)-6-(1-methylcyclopropoxy)-9H-purin-8-yl)phenoxy)-2,2-
dimethylpentanoic acid
4-(3-chloro-4-(9-(3-chlorobenzyl)-6-(1-methylcyclopropoxy)-9H-purin-8-yl)phenoxy)-2-
isopropylbutanoic acid (racemic)
9-benzyl-8-(2-chloro-4-(2-(2-(difluoromethyl)piperazin-1-yl)ethoxy)phenyl)-6-(1-
methylcyclopropoxy)-9H-purine
(R)-1-(3-(3-chloro-4-(9-((4-(difluoromethyl)pyridin-2-yl)methyl)-6-(1-methylcyclopropoxy)-
9H-purin-8-yl)phenoxy)propyl)pyrrolidin-3-ol
4-(3-chloro-4-(9-(2,5-dimethylbenzyl)-6-(1-methylcyclopropoxy)-9H-purin-8-yl)phenoxy)-2-
methylbutanoic acid (racemic)
4-(3-chloro-4-(9-(2,3-dimethylbenzyl)-6-(1-methylcyclopropoxy)-9H-purin-8-yl)phenoxy)-2-
methylbutanoic acid (racemic)
4-(3-chloro-4-(9-(2,6-dimethylbenzyl)-6-(1-methylcyclopropoxy)-9H-purin-8-yl)phenoxy)-2-
methylbutanoic acid (racemic)
4-(3-chloro-4-(9-(2,5-dimethylbenzyl)-6-(1-methylcyclopropoxy)-9H-purin-8-yl)phenoxy)-2-
methylbutanoic acid (Isomer 1)
4-(3-chloro-4-(9-(2,5-dimethylbenzyl)-6-(1-methylcyclopropoxy)-9H-purin-8-yl)phenoxy)-2-
methylbutanoic acid (Isomer 2)
2-(3-chloro-4-(6-(1-methylcyclopropoxy)-9-((4-methylpyridin-2-yl)methyl)-9H-purin-8-
yl)phenyl)-1-(4-hydroxypiperidin-1-yl)ethan-1-one
(S)-2-(3-chloro-4-(6-(1-methylcyclopropoxy)-9-((4-methylpyridin-2-yl)methyl)-9H-purin-8-
yl)phenyl)-1-(3-hydroxy-3-methylpyrrolidin-1-yl)ethan-1-one
(S)-2-(3-chloro-4-(6-(1-methylcyclopropoxy)-9-((4-methylpyridin-2-yl)methyl)-9H-purin-8-
yl)phenoxy)-1-(2-(hydroxymethyl)pyrrolidin-1-yl)ethan-1-one
(S)-2-(3-chloro-4-(6-(1-methylcyclopropoxy)-9-((4-methylpyridin-2-yl)methyl)-9H-purin-8-
yl)phenoxy)-1-(3-methoxypyrrolidin-1-yl)ethan-1-one
(R)-2-(3-chloro-4-(6-(1-methylcyclopropoxy)-9-((4-methylpyridin-2-yl)methyl)-9H-purin-8-
yl)phenoxy)-1-(3-(hydroxymethyl)pyrrolidin-1-yl)ethan-1-one
(R)-2-(3-chloro-4-(6-(1-methylcyclopropoxy)-9-((4-methylpyridin-2-yl)methyl)-9H-purin-8-
yl)phenoxy)-1-(2-(hydroxymethyl)pyrrolidin-1-yl)ethan-1-one
2-(3-chloro-4-(6-(1-methylcyclopropoxy)-9-((4-methylpyridin-2-yl)methyl)-9H-purin-8-
yl)phenoxy)-1-(4-hydroxypiperidin-1-yl)ethan-1-one
(R)-2-(3-chloro-4-(6-(1-methylcyclopropoxy)-9-((4-methylpyridin-2-yl)methyl)-9H-purin-8-
yl)phenoxy)-1-(3-hydroxy-3-methylpyrrolidin-1-yl)ethan-1-one
2-(3-chloro-4-(6-(1-methylcyclopropoxy)-9-((4-methylpyridin-2-yl)methyl)-9H-purin-8-
yl)phenoxy)-N-((1s,3s)-3-hydroxycyclobutyl)-N-methylacetamide
2-(3-chloro-4-(6-(1-methylcyclopropoxy)-9-((4-methylpyridin-2-yl)methyl)-9H-purin-8-
yl)phenoxy)-N-((1-hydroxycyclopropyl)methyl)-N-methylacetamide
(R)-1-((S)-3-(3-chloro-4-(6-(1-methylcyclopropoxy)-9-((4-methylpyridin-2-yl)methyl)-9H-
purin-8-yl)phenoxy)pyrrolidin-1-yl)-2-hydroxypropan-1-one
(S)-8-(2-chloro-4-(hexahydropyrrolo[1,2-a]pyrazin-2(1H)-yl)phenyl)-6-(1-
methylcyclopropoxy)-9-((4-methylpyridin-2-yl)methyl)-9H-purine
8-(2-chloro-4-(1,7-diazaspiro[3.5]nonan-7-yl)phenyl)-6-(1-methylcyclopropoxy)-9-((4-
methylpyridin-2-yl)methyl)-9H-purine
8-(4-(2-(1,6-diazaspiro[3.3]heptan-6-yl)ethoxy)-2-chlorophenyl)-6-(1-methylcyclopropoxy)-9-
((4-methylpyridin-2-yl)methyl)-9H-purine
(S)-(3-chloro-4-(6-(1-methylcyclopropoxy)-9-((4-methylpyridin-2-yl)methyl)-9H-purin-8-
yl)phenyl)(3-(dimethylamino)pyrrolidin-1-yl)methanone
2-(3-chloro-4-(6-(1-methylcyclopropoxy)-9-((4-methylpyridin-2-yl)methyl)-9H-purin-8-
yl)phenyl)-1-(1,4-diazepan-1-yl)ethan-1-one
8-(2-chloro-4-(3-(pyrrolidin-1-yl)propoxy)phenyl)-6-(1-methylcyclopropoxy)-9-((4-
methylpyridin-2-yl)methyl)-9H-purine
1-(3-chloro-4-(6-(1-methylcyclopropoxy)-9-((4-methylpyridin-2-yl)methyl)-9H-purin-8-
yl)phenethyl)piperidin-4-ol
(R)-1-(3-(3-chloro-4-(6-(1-methylcyclopropoxy)-9-((4-methylpyridin-2-yl)methyl)-9H-purin-8-
yl)phenoxy)propyl)pyrrolidin-3-ol
(R)-1-(3-(2-chloro-3-(6-(1-methylcyclopropoxy)-9-((4-methylpyridin-2-yl)methyl)-9H-purin-8-
yl)phenoxy)propyl)pyrrolidin-3-ol
(S)-8-(2-chloro-4-(2-(2-methylpiperazin-1-yl)ethoxy)phenyl)-6-(1-methylcyclopropoxy)-9-((4-
methylpyridin-2-yl)methyl)-9H-purine
8-(2-chloro-4-(2-(piperazin-1-yl)ethoxy)phenyl)-9-((4,6-dimethylpyridin-2-yl)methyl)-6-(1-
methylcyclopropoxy)-9H-purine
8-(2-chloro-4-(2-(piperazin-1-yl)ethoxy)phenyl)-9-((4,5-dimethylpyridin-2-yl)methyl)-6-(1-
methylcyclopropoxy)-9H-purine
8-(2-chloro-4-(2-(piperazin-1-yl)ethoxy)phenyl)-9-((3,4-dimethylpyridin-2-yl)methyl)-6-(1-
methylcyclopropoxy)-9H-purine
4-(3-chloro-4-(9-(2-cyclopropylbenzyl)-6-(1-methylcyclopropoxy)-9H-purin-8-yl)phenoxy)-2-
methylbutanoic acid (racemic)
4-(3-chloro-4-(9-(2-cyclopropylbenzyl)-6-(1-methylcyclopropoxy)-9H-purin-8-yl)phenoxy)-2-
methylbutanoic acid (Isomer 1)
4-(3-chloro-4-(9-(2-cyclopropylbenzyl)-6-(1-methylcyclopropoxy)-9H-purin-8-yl)phenoxy)-2-
methylbutanoic acid (Isomer 2)
4-(3-chloro-4-(9-(3-cyclopropylbenzyl)-6-(1-methylcyclopropoxy)-9H-purin-8-yl)phenoxy)-2-
methylbutanoic acid (racemic)
(3-chloro-4-(6-(1-methylcyclopropoxy)-9-((4-methylpyridin-2-yl)methyl)-9H-purin-8-
yl)phenyl)(3-(pyrrolidin-1-yl)azetidin-1-yl)methanone
1-([1,3′-biazetidin]-1′-yl)-2-(3-chloro-4-(6-(1-methylcyclopropoxy)-9-((4-methylpyridin-2-
yl)methyl)-9H-purin-8-yl)phenyl)ethan-1-one
rel-(1R,4R)-2-(2-(4-(9-benzyl-6-(1-methylcyclopropoxy)-9H-purin-8-yl)-3-
chlorophenoxy)ethyl)-2,5-diazabicyclo[2.2.2]octane (unknown absolute stereochemistry)
8-(4-(2-(3,8-diazabicyclo[3.2.1]octan-8-yl)ethoxy)-2-chlorophenyl)-9-benzyl-6-(1-
methylcyclopropoxy)-9H-purine
2-(3-chloro-4-(6-(1-methylcyclopropoxy)-9-((4-methylpyridin-2-yl)methyl)-9H-purin-8-
yl)phenyl)-1-((3R,5S)-3,5-dimethylpiperazin-1-yl)ethan-1-one
1-(4-(2-(3-chloro-4-(6-(1-methylcyclopropoxy)-9-((4-methylpyridin-2-yl)methyl)-9H-purin-8-
yl)phenoxy)ethyl)piperazin-1-yl)ethan-1-one
4-(tert-butoxy)-2-(2-chloro-4-(2-(piperazin-1-yl)ethoxy)phenyl)-1-(3-chlorobenzyl)-1H-
benzo[d]imidazole
4-(tert-butoxy)-2-(2-chloro-4-(2-(piperazin-1-yl)ethoxy)phenyl)-1-((4-methylpyridin-2-
yl)methyl)-1H-benzo[d]imidazole
4-(tert-butoxy)-2-(2-chloro-4-(2-(4-methylpiperazin-1-yl)ethoxy)phenyl)-1-((4-methylpyridin-
2-yl)methyl)-1H-imidazo[4,5-c]pyridine
2-(2-chloro-4-(2-(piperazin-1-yl)ethoxy)phenyl)-1-(3-chlorobenzyl)-4-(1-methylcyclobutoxy)-
1H-benzo[d]imidazole
9-benzyl-8-(2-(methoxymethyl)-4-(2-(4-methylpiperazin-1-yl)ethoxy)phenyl)-6-(1-
methylcyclopropoxy)-9H-purine
4-(3-chloro-4-(9-(2-methoxy-5-methylbenzyl)-6-(1-methylcyclopropoxy)-9H-purin-8-
yl)phenoxy)-2-methylbutanoic acid (racemic)
4-(3-chloro-4-(9-(5-fluoro-2-methoxybenzyl)-6-(1-methylcyclopropoxy)-9H-purin-8-
yl)phenoxy)-2-methylbutanoic acid (racemic)
4-(3-chloro-4-(9-(3-chloro-2-methoxybenzyl)-6-(1-methylcyclopropoxy)-9H-purin-8-
yl)phenoxy)-2-methylbutanoic acid (racemic)
4-(3-chloro-4-(9-(2-methoxybenzyl)-6-(1-methylcyclopropoxy)-9H-purin-8-yl)phenoxy)-2-
methylbutanoic acid (racemic)
4-(3-chloro-4-(9-(2-fluorobenzyl)-6-(1-methylcyclopropoxy)-9H-purin-8-yl)phenoxy)-2-
methylbutanoic acid (racemic)
4-(3-chloro-4-(9-(3-fluorobenzyl)-6-(1-methylcyclopropoxy)-9H-purin-8-yl)phenoxy)-2-
methylbutanoic acid (racemic)
4-(3-chloro-4-(9-(4-cyanobenzyl)-6-(1-methylcyclopropoxy)-9H-purin-8-yl)phenoxy)-2-
methylbutanoic acid (racemic)
(S)-4-(3-chloro-4-(9-(3-cyanobenzyl)-6-(1-methylcyclopropoxy)-9H-purin-8-yl)phenoxy)-2-
methylbutanoic acid
4-(3-chloro-4-(9-(5-chloro-2-cyanobenzyl)-6-(1-methylcyclopropoxy)-9H-purin-8-yl)phenoxy)-
2-methylbutanoic acid (racemic)
(R)-4-(3-chloro-4-(9-(5-chloro-2-fluorobenzyl)-6-(1-methylcyclopropoxy)-9H-purin-8-
yl)phenoxy)-2-methylbutanoic acid
(S)-4-(3-chloro-4-(9-(5-chloro-2-fluorobenzyl)-6-(1-methylcyclopropoxy)-9H-purin-8-
yl)phenoxy)-2-methylbutanoic acid
5-(3-chloro-4-(9-(2-cyanobenzyl)-6-(1-methylcyclopropoxy)-9H-purin-8-yl)phenyl)-2-
methylpentanoic acid (racemic)
4-(3-chloro-4-(9-(2-cyano-6-fluorobenzyl)-6-(1-methylcyclopropoxy)-9H-purin-8-yl)phenoxy)-
2-methylbutanoic acid (racemic)
4-(3-chloro-4-(9-(5-cyano-2-methoxybenzyl)-6-(1-methylcyclopropoxy)-9H-purin-8-
yl)phenoxy)-2-methylbutanoic acid (racemic)
2-(1-((3-chloro-4-(9-(2-cyanobenzyl)-6-(1-methylcyclopropoxy)-9H-purin-8-
yl)phenoxy)methyl)cyclopropyl)acetic acid
(1s,4s)-4-(3-chloro-4-(9-(2-cyanobenzyl)-6-(1-methylcyclopropoxy)-9H-purin-8-
yl)phenoxy)cyclohexane-1-carboxylic acid
4-(3-chloro-4-(9-(2-cyanobenzyl)-6-(1-methylcyclopropoxy)-9H-purin-8-yl)phenoxy)-2,2-
dimethylbutanoic acid
4-(3-chloro-4-(9-(5-cyano-2-fluorobenzyl)-6-(1-methylcyclopropoxy)-9H-purin-8-yl)phenoxy)-
2-methylbutanoic acid (racemic)
(1r,4r)-4-(3-chloro-4-(9-(2-cyanobenzyl)-6-(1-methylcyclopropoxy)-9H-purin-8-
yl)phenoxy)cyclohexane-1-carboxylic acid
5-(3-chloro-4-(9-(2-cyanobenzyl)-6-(1-methylcyclopropoxy)-9H-purin-8-yl)phenyl)pentanoic
acid
5-(3-chloro-4-(9-(2-cyanobenzyl)-6-(1-methylcyclopropoxy)-9H-purin-8-yl)phenoxy)-2,2-
dimethylpentanoic acid
2-(2-(2-chloro-3-(9-(5-chloro-2-fluorobenzyl)-6-(1-methylcyclopropoxy)-9H-purin-8-
yl)phenoxy)ethoxy)acetic acid
(S)-4-(2-chloro-3-(9-(5-chloro-2-fluorobenzyl)-6-(1-methylcyclopropoxy)-9H-purin-8-
yl)phenoxy)-2-methylbutanoic acid
4-(3-chloro-4-(9-(2-cyanobenzyl)-6-(1-methylcyclopropoxy)-9H-purin-8-yl)phenoxy)-3-
methylbutanoic acid (racemic)
(R)-4-(2-chloro-3-(9-(5-chloro-2-fluorobenzyl)-6-(1-methylcyclopropoxy)-9H-purin-8-
yl)phenoxy)-2-methylbutanoic acid
(1r,3r)-3-((3-chloro-4-(9-(2-cyanobenzyl)-6-(1-methylcyclopropoxy)-9H-purin-8-
yl)phenoxy)methyl)cyclobutane-1-carboxylic acid
5-(2-chloro-3-(9-(3-chlorobenzyl)-6-(1-methylcyclopropoxy)-9H-purin-8-yl)phenoxy)-3,3-
dimethylpentanoic acid
2-(3-(2-chloro-3-(9-(5-chloro-2-methoxybenzyl)-6-(1-methylcyclopropoxy)-9H-purin-8-
yl)phenoxy)propoxy)acetic acid
4-(3-chloro-4-(9-(5-chloro-2-methoxybenzyl)-6-(1-methylcyclopropoxy)-9H-purin-8-
yl)phenoxy)-2,2-dimethylbutanoic acid
3-(2-chloro-3-(9-(5-chloro-2-fluorobenzyl)-6-(1-methylcyclopropoxy)-9H-purin-8-
yl)phenoxy)propanoic acid
4-(2-chloro-3-(9-(5-chloro-2-fluorobenzyl)-6-(1-methylcyclopropoxy)-9H-purin-8-yl)phenoxy)-
3-methylbutanoic acid (isomer 1)
4-(2-chloro-3-(9-(5-chloro-2-fluorobenzyl)-6-(1-methylcyclopropoxy)-9H-purin-8-yl)phenoxy)-
3-methylbutanoic acid (isomer 2)
4-(3-chloro-4-(9-(3-chloro-2-fluoro-5-(trifluoromethyl)benzyl)-6-(1-methylcyclopropoxy)-9H-
purin-8-yl)phenoxy)-2-methylbutanoic acid (racemic)
4-(3-chloro-4-(9-(5-chloro-2-methylbenzyl)-6-(1-methylcyclopropoxy)-9H-purin-8-
yl)phenoxy)-2-methylbutanoic acid (racemic)
4-(3-chloro-4-(9-(3-chloro-2-cyanobenzyl)-6-(1-methylcyclopropoxy)-9H-purin-8-yl)phenoxy)-
2-methylbutanoic acid (racemic)
(1r,4r)-4-(2-chloro-3-(9-(5-chloro-2-methoxybenzyl)-6-(1-methylcyclopropoxy)-9H-purin-8-
yl)phenoxy)cyclohexane-1-carboxylic acid
3-(2-chloro-3-(9-(3-chlorobenzyl)-6-(1-methylcyclopropoxy)-9H-purin-8-yl)-5-
fluorophenoxy)propanoic acid
(1s,4s)-4-(3-chloro-4-(9-(5-chloro-2-methoxybenzyl)-6-(1-methylcyclopropoxy)-9H-purin-8-
yl)phenoxy)cyclohexane-1-carboxylic acid
4-(3-chloro-4-(9-(5-chloro-2-methoxybenzyl)-6-(1-methylcyclopropoxy)-9H-purin-8-
yl)phenoxy)-3-methylbutanoic acid (racemic)
(1r,4r)-4-(3-chloro-4-(9-(5-chloro-2-methoxybenzyl)-6-(1-methylcyclopropoxy)-9H-purin-8-
yl)phenoxy)cyclohexane-1-carboxylic acid
(1s,4s)-4-(3-chloro-4-(9-(5-chloro-2-fluorobenzyl)-6-(1-methylcyclopropoxy)-9H-purin-8-
yl)phenoxy)cyclohexane-1-carboxylic acid
4-(3-chloro-4-(9-(5-chloro-2-fluorobenzyl)-6-(1-methylcyclopropoxy)-9H-purin-8-yl)phenoxy)-
3-methylbutanoic acid (racemic)
4-(3-chloro-4-(9-(5-chloro-2-fluorobenzyl)-6-(1-methylcyclopropoxy)-9H-purin-8-yl)phenoxy)-
2,2-dimethylbutanoic acid
5-(3-chloro-4-(9-(5-chloro-2-methoxybenzyl)-6-(1-methylcyclopropoxy)-9H-purin-8-
yl)phenoxy)-2,2-dimethylpentanoic acid
2-(2-(3-chloro-4-(9-(5-chloro-2-methoxybenzyl)-6-(1-methylcyclopropoxy)-9H-purin-8-
yl)phenoxy)ethoxy)acetic acid
(1r,4r)-4-(3-chloro-4-(9-(5-chloro-2-fluorobenzyl)-6-(1-methylcyclopropoxy)-9H-purin-8-
yl)phenoxy)cyclohexane-1-carboxylic acid
(1s,4s)-4-(2-chloro-3-(9-(5-chloro-2-methoxybenzyl)-6-(1-methylcyclopropoxy)-9H-purin-8-
yl)phenoxy)cyclohexane-1-carboxylic acid
(1r,3r)-3-(2-chloro-3-(9-(5-chloro-2-methoxybenzyl)-6-(1-methylcyclopropoxy)-9H-purin-8-
yl)phenoxy)cyclobutane-1-carboxylic acid
2-(2-(2-chloro-3-(9-(5-chloro-2-methoxybenzyl)-6-(1-methylcyclopropoxy)-9H-purin-8-
yl)phenoxy)ethoxy)acetic acid
4-(3-chloro-4-(9-((5-chloro-2-methoxypyridin-3-yl)methyl)-6-(1-methylcyclopropoxy)-9H-
purin-8-yl)phenoxy)-2-methylbutanoic acid (racemic)
2-(2-(3-chloro-4-(9-(5-chloro-2-methoxybenzyl)-6-(1-methylcyclopropoxy)-9H-purin-8-
yl)phenoxy)ethoxy)-2-methylpropanoic acid
5-(2-chloro-3-(9-(5-chloro-2-methoxybenzyl)-6-(1-methylcyclopropoxy)-9H-purin-8-
yl)phenyl)-2-methylpentanoic acid (racemic)
5-(3-chloro-4-(9-(5-chloro-2-methoxybenzyl)-6-(1-methylcyclopropoxy)-9H-purin-8-
yl)phenyl)-2-methylpentanoic acid (racemic)
4-(3-chloro-4-(9-(3-chloro-2-fluoro-6-methoxybenzyl)-6-(1-methylcyclopropoxy)-9H-purin-8-
yl)phenoxy)-2-methylbutanoic acid (racemic)
4-(3-chloro-4-(9-(5-chloro-2-ethoxybenzyl)-6-(1-methylcyclopropoxy)-9H-purin-8-
yl)phenoxy)-2-methylbutanoic acid (racemic)
4-(3-chloro-4-(9-(5-chloro-2-(difluoromethoxy)benzyl)-6-(1-methylcyclopropoxy)-9H-purin-8-
yl)phenoxy)-2-methylbutanoic acid (racemic)
4-(3-chloro-4-(9-(5-chloro-3-fluoro-2-methoxybenzyl)-6-(1-methylcyclopropoxy)-9H-purin-8-
yl)phenoxy)-2-methylbutanoic acid (racemic)
4-(2-chloro-3-(9-(5-chloro-2-methoxybenzyl)-6-(1-methylcyclopropoxy)-9H-purin-8-
yl)phenyl)butanoic acid
4-(2-chloro-3-(9-(3-chlorobenzyl)-6-(1-methylcyclopropoxy)-9H-purin-8-yl)phenoxy)-3,3-
difluorobutanoic acid
3-(2-chloro-3-(9-(5-chloro-2-methoxybenzyl)-6-(1-methylcyclopropoxy)-9H-purin-8-
yl)phenyl)propanoic acid
3-(2-chloro-3-(9-(3-chlorobenzyl)-6-(1-methylcyclopropoxy)-9H-purin-8-yl)-6-
fluorophenoxy)propanoic acid
3-(2-chloro-3-(9-(3-chlorobenzyl)-6-(1-methylcyclopropoxy)-9H-purin-8-yl)-4-
fluorophenoxy)propanoic acid
(R)-4-(3-chloro-4-(9-(5-fluoro-2-methoxybenzyl)-6-(1-methylcyclopropoxy)-9H-purin-8-
yl)phenoxy)-2-methylbutanoic acid
(S)-4-(3-chloro-4-(9-(5-fluoro-2-methoxybenzyl)-6-(1-methylcyclopropoxy)-9H-purin-8-
yl)phenoxy)-2-methylbutanoic acid
(1r,4r)-4-(2-chloro-3-(9-(5-fluoro-2-methoxybenzyl)-6-(1-methylcyclopropoxy)-9H-purin-8-
yl)phenoxy)cyclohexane-1-carboxylic acid
2-(3-(2-chloro-3-(9-(5-fluoro-2-methoxybenzyl)-6-(1-methylcyclopropoxy)-9H-purin-8-
yl)phenoxy)propoxy)acetic acid
(S)-4-(2-chloro-3-(9-(5-fluoro-2-methoxybenzyl)-6-(1-methylcyclopropoxy)-9H-purin-8-
yl)phenoxy)-2-methylbutanoic acid
(S)-4-(2-chloro-3-(9-(5-chloro-2-methoxybenzyl)-6-(1-methylcyclopropoxy)-9H-purin-8-
yl)phenoxy)-2-methylbutanoic acid
(1s,3s)-3-(2-chloro-3-(9-(5-fluoro-2-methoxybenzyl)-6-(1-methylcyclopropoxy)-9H-purin-8-
yl)phenoxy)cyclobutane-1-carboxylic acid
(R)-4-(2-chloro-3-(9-(5-chloro-2-methoxybenzyl)-6-(1-methylcyclopropoxy)-9H-purin-8-
yl)phenoxy)-2-methylbutanoic acid
(S)-4-(3-chloro-4-(9-(2-methoxy-5-(trifluoromethyl)benzyl)-6-(1-methylcyclopropoxy)-9H-
purin-8-yl)phenoxy)-2-methylbutanoic acid
4-(2-chloro-3-(9-(5-fluoro-2-methoxybenzyl)-6-(1-methylcyclopropoxy)-9H-purin-8-
yl)phenoxy)-3-methylbutanoic acid (Isomer1)
(R)-4-(2-chloro-3-(9-(5-fluoro-2-methoxybenzyl)-6-(1-methylcyclopropoxy)-9H-purin-8-
yl)phenoxy)-2-methylbutanoic acid
(R)-4-(3-chloro-4-(9-(2-methoxy-5-(trifluoromethyl)benzyl)-6-(1-methylcyclopropoxy)-9H-
purin-8-yl)phenoxy)-2-methylbutanoic acid
4-(2-chloro-3-(9-(5-fluoro-2-methoxybenzyl)-6-(1-methylcyclopropoxy)-9H-purin-8-
yl)phenoxy)-3-methylbutanoic acid (Isomer 2)
(R)-4-(3-chloro-4-(9-(5-chloro-2-cyanobenzyl)-6-(1-methylcyclopropoxy)-9H-purin-8-
yl)phenoxy)-2-methylbutanoic acid
(S)-4-(3-chloro-4-(9-(5-chloro-2-cyanobenzyl)-6-(1-methylcyclopropoxy)-9H-purin-8-
yl)phenoxy)-2-methylbutanoic acid
3-(2-chloro-3-(9-(5-fluoro-2-methoxybenzyl)-6-(1-methylcyclopropoxy)-9H-purin-8-
yl)phenoxy)propanoic acid
(1s,3s)-3-(2-chloro-3-(9-(5-chloro-2-cyanobenzyl)-6-(1-methylcyclopropoxy)-9H-purin-8-
yl)phenoxy)cyclobutane-1-carboxylic acid
(1r,4r)-4-(2-chloro-3-(9-(5-chloro-2-cyanobenzyl)-6-(1-methylcyclopropoxy)-9H-purin-8-
yl)phenoxy)cyclohexane-1-carboxylic acid
(S)-(4-(3-chloro-4-(9-(3-chlorobenzyl)-6-(1-methylcyclopropoxy)-9H-purin-8-yl)phenoxy)-2-
methylbutanoyl)glycine
(S)-4-(2-chloro-3-(9-(5-chloro-2-cyanobenzyl)-6-(1-methylcyclopropoxy)-9H-purin-8-
yl)phenoxy)-2-methylbutanoic acid
3-(2-chloro-3-(9-(2-methoxy-5-methylbenzyl)-6-(1-methylcyclopropoxy)-9H-purin-8-
yl)phenoxy)propanoic acid
2-(3-(2-chloro-3-(9-(5-chloro-2-methoxybenzyl)-6-(1-methylcyclopropoxy)-9H-purin-8-
yl)phenoxy)propoxy)-2-methylpropanoic acid
(1r,4r)-4-(2-chloro-3-(9-(2-methoxy-5-methylbenzyl)-6-(1-methylcyclopropoxy)-9H-purin-8-
yl)phenoxy)cyclohexane-1-carboxylic acid
2-(3-(3-chloro-4-(9-(5-chloro-2-methoxybenzyl)-6-(1-methylcyclopropoxy)-9H-purin-8-
yl)phenoxy)propoxy)-2-methylpropanoic acid
2-(3-(2-chloro-3-(9-(2-methoxy-5-methylbenzyl)-6-(1-methylcyclopropoxy)-9H-purin-8-
yl)phenoxy)propoxy)acetic acid
(S)-4-(3-chloro-4-(9-(2-methoxy-5-methylbenzyl)-6-(1-methylcyclopropoxy)-9H-purin-8-
yl)phenoxy)-2-methylbutanoic acid
(R)-4-(2-chloro-3-(9-(5-chloro-2-cyanobenzyl)-6-(1-methylcyclopropoxy)-9H-purin-8-
yl)phenoxy)-2-methylbutanoic acid
3-(2-chloro-3-(9-(5-chloro-2-cyanobenzyl)-6-(1-methylcyclopropoxy)-9H-purin-8-
yl)phenoxy)propanoic acid
2-(3-(2-chloro-3-(9-(5-chloro-2-cyanobenzyl)-6-(1-methylcyclopropoxy)-9H-purin-8-
yl)phenoxy)propoxy)acetic acid
4-(2-chloro-3-(9-(2-methoxy-5-methylbenzyl)-6-(1-methylcyclopropoxy)-9H-purin-8-
yl)phenoxy)-3-methylbutanoic acid (isomer 2)
4-(2-chloro-3-(9-(2-methoxy-5-methylbenzyl)-6-(1-methylcyclopropoxy)-9H-purin-8-
yl)phenoxy)-3-methylbutanoic acid (Isomer 1)
(1s,3s)-3-(2-chloro-3-(9-(2-methoxy-5-methylbenzyl)-6-(1-methylcyclopropoxy)-9H-purin-8-
yl)phenoxy)cyclobutane-1-carboxylic acid
(S)-4-(2-chloro-3-(9-(2-methoxy-5-methylbenzyl)-6-(1-methylcyclopropoxy)-9H-purin-8-
yl)phenoxy)-2-methylbutanoic acid
4-(2-chloro-3-(9-(5-chloro-2-cyanobenzyl)-6-(1-methylcyclopropoxy)-9H-purin-8-yl)phenoxy)-
3-methylbutanoic acid (Isomer 1)
4-(2-chloro-3-(9-(5-chloro-2-cyanobenzyl)-6-(1-methylcyclopropoxy)-9H-purin-8-yl)phenoxy)-
3-methylbutanoic acid (Isomer 2)
(R)-4-(2-chloro-3-(9-(2-methoxy-5-methylbenzyl)-6-(1-methylcyclopropoxy)-9H-purin-8-
yl)phenoxy)-2-methylbutanoic acid

In some embodiments, the inhibitor of POL Q is 9-Benzyl-8-(2-chloro-4-(2-(4-methylpiperazin-1-yl)ethoxy)phenyl)-6-(1-methylcyclopropoxy)-9H-purine (Compound 1), or a salt thereof. In some embodiments, the inhibitor of POL Q is 9-Benzyl-8-(2-chloro-4-(2-(4-methylpiperazin-1-yl)ethoxy)phenyl)-6-(1-methylcyclopropoxy)-9H-purine (Compound 1)

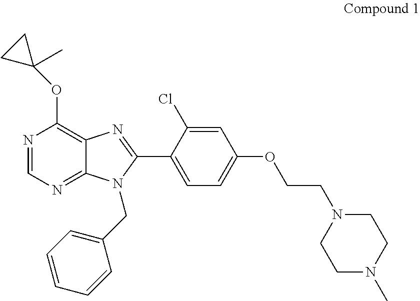 [SMILES: CN1CCN(CCOc2ccc(-c3nc4c(OC5(C)CC5)ncnc4n3Cc3ccccc3)c(Cl)c2)CC1]

In some embodiments, the inhibitor of POL Q is a compound disclosed herein, a pharmaceutically acceptable salt thereof, or combinations thereof. In some embodiments, the inhibitor of POL Q is a compound disclosed in Table I, II, III, a pharmaceutically acceptable salt thereof. or combinations thereof. In some embodiments, the inhibitor of POL Q is Compound 1 or a pharmaceutical salt thereof. In some embodiments, the inhibitor of POL Q is Compound 1.

In some embodiments, the inhibitor of the MMEJ pathway is added to the composition comprising the eukaryotic cell at a concentration of about 0.01 mM to about 1 mM. In some embodiments the concentration of the inhibitor of the MMEJ pathway is about 0.01 mM to about 0.75 mM, about 0.01 mM to about 0.5 mM, about 0.01 mM to about 0.25 mM, about 0.01 mM to about 0.1 mM, about 0.01 mM to about 75 mM, about 0.01 mM to about 50 mM, about 0.01 mM to about 25 mM, about 0.01 to about 25 mM, about 0.01 to about 20 mM, about 0.01 mM to about 15 mM, about 0.01 mM to about 10 mM, or about 0.01 mM to about 1 mM. In some embodiments the concentration of the inhibitor of the MMEJ pathway is about 0.1 mM to about 1 mM, about 1 mM to about 1 mM, about 10 mM to about 1 mM, about 15 mM to about 1 M, about 20 mM to about 1 M, about 25 mM to about 1 mM, about 50 mM to about 1 mM, about 75 mM to about 1 mM, about 0.1 mM to about 1 mM, about 0.25 mM to about 1 mM, about 0.5 mM to about 1 mM, or about 0.75 mM to about 1 mM. In some embodiments, the concentration of the inhibitor of the MMEJ pathway is about 0.1 mM to about 1 mM, 0.1 mM to about 0.75 mM, about 0.1 mM to about 0.5 mM, about 0.1 mM to about 0.25 mM, about 0.1 mM to about 0.1 mM, about 0.1 mM to about 75 mM, about 0.1 mM to about 50 mM, about 0.1 mM to about 25 mM, about 0.1 mM to about 20 mM, about 0.1 mM to about 15 mM, about 0.1 mM to about 10 mM, or about 0.1 mM to about 1 mM. In some embodiments, the concentration of the inhibitor of the MMEJ pathway is about 1 mM to about 10 mM, about 1 mM to about 15 mM, about 1 mM to about 20 mM, about 1 mM to about 25 mM, about 1 mM to about 50 mM, about 1 mM to about 0.1 mM, about 1 mM to about 0.25 mM, about 1 mM to about 0.5 mM, about 1 mM to about 0.75 mM, or about 1 mM to about 1 mM. In some embodiments, the concentration of the inhibitor of the MMEJ pathway is about 0.01 mM to about 100 mM, about 0.1 mM to about 90 mM, about 0.2 mM to about 80 mM, about 0.3 mM to about 70 mM, about 0.4 mM to about 60 mM, about 0.5 mM to about 50 mM, about 1 mM to about 50 mM, about 2 mM to about 45 mM, about 3 mM to about 40 mM, about 4 mM to about 35 mM, about 5 mM to about 30 mM, about 6 mM to about 25 mM, about 7 mM to about 20 mM, or about 8 mM to about 15 mM. In some embodiments, the concentration of the inhibitor of the MMEJ pathway is about 0.01 mM to about 0.1 mM, about 0.01 to about 1 mM, about 0.05 mM to about 0.1 mM, about 0.5 mM to about 1 mM, about 0.5 mM to about 5 mM, about 0.5 mM to about 10 mM, about 0.1 mM to about 1 mM, about 0.1 mM to about 5 mM, about 0.1 mM to about 10 mM, about 1 mM to about 5 mM, about 1 mM to about 10 mM, about 1 mM to about 15 mM, about 1 mM to about 20 mM, about 1 mM to about 25 mM, about 1 mM to about 50 mM, about 5 mM to about 10 mM, about 5 mM to about 15 mM, about 5 mM to about 20 mM, or about 5 mM to about 25 mM. In some embodiments, the concentration of the inhibitor of the MMEJ pathway is about 0.1, 0.2, 0.3, 0.4, 0.5, 0.6, 0.7, 0.7, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mM.

In some embodiments, the concentration of the inhibitor of the MMEJ pathway is 0.01 mM to about 1 mM, about 0.1 mM to about 1 mM, about 0.1 mM to about 0.5 mM, about 0.1 mM to about 100 mM, or about 1 mM to about 50 mM.

In some embodiments, the inhibitor of the MMEJ pathway is added to the composition comprising the eukaryotic cell about 0 minutes to about 96 hours before the Cas effector protein is added, about 0 minutes to about 72 hours before the Cas effector protein is added, about 0 minutes to about 48 hours before the Cas effector protein is added, about 0 minutes to about 36 hours before the Cas effector protein is added, about 0 minutes to about 24 hours before the Cas effector protein is added, about 0 minutes to about 18 hours before the Cas effector protein is added, about 0 minutes to about 12 hours before the Cas effector protein is added, about 0 minutes to about 6 hours before the Cas effector protein is added, about 0 minutes to about 3 hours before the Cas effector protein is added, about 0 minutes to about 2 hours before the Cas effector protein is added, about 0 minutes to about 1 hour before the Cas effector protein is added, or about 0 minutes to about 30 minutes before the Cas effector protein is added. In some embodiments, the inhibitor of the MMEJ pathway is added to the composition comprising a eukaryotic cell about 1, 2, 3, 4, 5, 6, 7, 8, 9, 10, 11, 12, 13, 14, 15, 16, 17, 18, 19, 20, 21, 22, 23, 24, 24, 25, 26, 27, 28, 29, 30, 31, 32, 33, 34, 35, 40, 45, 50, 55, 60, 65, 70, 75, 80, 85, 90, 95, or 100 hours before the Cas effector protein is added.

In some embodiments, the inhibitor of the MMEJ pathway is added to the composition comprising a eukaryotic cell at the same time the Cas effector protein is added.

In some embodiments, the inhibitor of the MMEJ pathway is added to the composition comprising a eukaryotic cell about 0 minutes to about 30 minutes after the Cas effector protein is added, about 0 minutes to about 1 hour after the Cas effector protein is added, about 0 minutes to about 3 hours after the Cas effector protein is added, about 0 minutes to about 6 hours after the Cas effector protein is added, about 0 minutes to about 12 hours after the Cas effector protein is added, about 0 minutes to about 18 hours after the Cas effector protein is added, about 0 minutes to about 24 hours after the Cas effector protein is added, about 0 minutes to about 36 hours after the Cas effector protein is added, about 0 minutes to about 48 hours after the Cas effector protein is added, about 0 minutes to about 72 hours after the Cas effector protein is added, or about 0 minutes to about 96 hours after the Cas effector protein is added. In some embodiments, the inhibitor of the MMEJ pathway is added to the composition comprising a eukaryotic cell about 1, 2, 3, 4, 5, 6, 7, 8, 9, 10, 11, 12, 13, 14, 15, 16, 17, 18, 19, 20, 21, 22, 23, 24, 24, 25, 26, 27, 28, 29, 30, 31, 32, 33, 34, 35, 40, 45, 50, 55, 60, 65, 70, 75, 80, 85, 90, 95, or 100 hours after the Cas effector protein is added.

In some embodiments, the inhibitor of the MMEJ pathway is in the composition comprising a eukaryotic cell for about 1 to about 300 hours, about 10 to about 200 hours, about 10 to about 100 hours, about 20 to about 80 hours, about 30 to about 70 hours, or about 40 to about hours. In some embodiments, the inhibitor of the MMEJ pathway is in the composition comprising a eukaryotic cell for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, 100, 125, 150, 175, 200, 225, 250, 275, or 300 hours.

In some embodiments, the inhibitor of the MMEJ pathway is added to the composition comprising a eukaryotic cell at least 1, 2, 3, 4, 5, 6, 7, 8, 9, or 10 or more times.

Inhibitors of the non-homologous end joining (NHEJ) pathway

As used herein, an inhibitor of the NHEJ pathway is any compound, molecule, or entity that inhibits, antagonizes, blocks, or decreases the activity and/or level of any component of the NHEJ pathway. The NHEJ inhibitor can be an antibody or antigen-binding fragment thereof, a peptide, soluble protein, siRNA, antisense oligonucleotide, aptamer, or small-molecule compound that inhibits, antagonizes, blocks, or decreases the activity and/or level of any component of the NHEJ pathway. In some embodiments, the NHEJ pathway inhibits, antagonizes, blocks, or decreases the activity and/or level of Ku70, Ku80, DNA Ligase IV, XLF (non-homologous end-joining factor 1; XRCC4-like factor), or DNA-dependent protein kinase (DNA-PK). In some embodiments, the inhibitor of DNA-PK is M3814, M9831/VX984, Nu7441, KU0060648, AZD7648, Nu5455, vanillin, wortmannin, or combinations thereof. In some embodiments, the inhibitor of DNA-PK is AZD7648.

In some embodiments, the inhibitor of the NHEJ pathway is added to the composition comprising the eukaryotic cell at a concentration of about 0.01 mM to about 1 mM. In some embodiments the concentration of the inhibitor of the NHEJ pathway is about 0.01 mM to about 0.75 mM, about 0.01 mM to about 0.5 mM, about 0.01 mM to about 0.25 mM, about 0.01 mM to about 0.1 mM, about 0.01 mM to about 75 mM, about 0.01 mM to about 50 mM, about 0.01 mM to about 25 mM, about 0.01 to about 25 mM, about 0.01 to about 20 mM, about 0.01 mM to about 15 mM, about 0.01 mM to about 10 mM, or about 0.01 mM to about 1 mM. In some embodiments the concentration of the inhibitor of the NHEJ pathway is about 0.1 mM to about 1 mM, about 1 mM to about 1 mM, about 10 mM to about 1 mM, about 15 mM to about 1 M, about 20 mM to about 1 M, about 25 mM to about 1 mM, about 50 mM to about 1 mM, about 75 mM to about 1 mM, about 0.1 mM to about 1 mM, about 0.25 mM to about 1 mM, about 0.5 mM to about 1 mM, or about 0.75 mM to about 1 mM. In some embodiments, the concentration of the inhibitor of the NHEJ pathway is about 0.1 mM to about 1 mM, 0.1 mM to about 0.75 mM, about 0.1 mM to about 0.5 mM, about 0.1 mM to about 0.25 mM, about 0.1 mM to about 0.1 mM, about 0.1 mM to about 75 mM, about 0.1 mM to about 50 mM, about 0.1 mM to about 25 mM, about 0.1 mM to about 20 mM, about 0.1 mM to about 15 mM, about 0.1 mM to about 10 mM, or about 0.1 mM to about 1 mM. In some embodiments, the concentration of the inhibitor of the NHEJ pathway is about 1 mM to about 10 mM, about 1 mM to about 15 mM, about 1 mM to about 20 mM, about 1 mM to about 25 mM, about 1 mM to about 50 mM, about 1 mM to about 0.1 mM, about 1 mM to about 0.25 mM, about 1 mM to about 0.5 mM, about 1 mM to about 0.75 mM, or about 1 mM to about 1 mM. In some embodiments, the concentration of the inhibitor of the NHEJ pathway is about 0.01 mM to about 100 mM, about 0.1 mM to about 90 mM, about 0.2 mM to about 80 mM, about 0.3 mM to about 70 mM, about 0.4 mM to about 60 mM, about 0.5 mM to about 50 mM, about 1 mM to about 50 mM, about 2 mM to about 45 mM, about 3 mM to about 40 mM, about 4 mM to about 35 mM, about 5 mM to about 30 mM, about 6 mM to about 25 mM, about 7 mM to about 20 mM, or about 8 mM to about 15 mM. In some embodiments, the concentration of the inhibitor of the NHEJ pathway is about 0.01 mM to about 0.1 mM, about 0.01 to about 1 mM, about 0.05 mM to about 0.1 mM, about 0.5 mM to about 1 mM, about 0.5 mM to about 5 mM, about 0.5 mM to about 10 mM, about 0.1 mM to about 1 mM, about 0.1 mM to about 5 mM, about 0.1 mM to about 10 mM, about 1 mM to about 5 mM, about 1 mM to about 10 mM, about 1 mM to about 15 mM, about 1 mM to about 20 mM, about 1 mM to about 25 mM, about 1 mM to about 50 mM, about 5 mM to about 10 mM, about 5 mM to about 15 mM, about 5 mM to about 20 mM, or about 5 mM to about 25 mM. In some embodiments, the concentration of the inhibitor of the NHEJ pathway is about 0.1, 0.2, 0.3, 0.4, 0.5, 0.6, 0.7, 0.7, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 mM.

In some embodiments, the concentration of the inhibitor of the NHEJ pathway is 0.01 mM to about 1 mM, about 0.1 mM to about 1 mM, about 0.1 mM to about 0.5 mM, about 0.1 mM to about 100 mM, or about 1 mM to about 50 mM.

In some embodiments, the inhibitor of the NHEJ pathway is added to the composition comprising the eukaryotic cell about 0 minutes to about 96 hours before the Cas effector protein is added, about 0 minutes to about 72 hours before the Cas effector protein is added, about 0 minutes to about 48 hours before the Cas effector protein is added, about 0 minutes to about 36 hours before the Cas effector protein is added, about 0 minutes to about 24 hours before the Cas effector protein is added, about 0 minutes to about 18 hours before the Cas effector protein is added, about 0 minutes to about 12 hours before the Cas effector protein is added, about 0 minutes to about 6 hours before the Cas effector protein is added, about 0 minutes to about 3 hours before the Cas effector protein is added, about 0 minutes to about 2 hours before the Cas effector protein is added, about 0 minutes to about 1 hour before the Cas effector protein is added, or about 0 minutes to about 30 minutes before the Cas effector protein is added. In some embodiments, the inhibitor of the NHEJ pathway is added to the composition comprising a eukaryotic cell about 1, 2, 3, 4, 5, 6, 7, 8, 9, 10, 11, 12, 13, 14, 15, 16, 17, 18, 19, 20, 21, 22, 23, 24, 24, 25, 26, 27, 28, 29, 30, 31, 32, 33, 34, 35, 40, 45, 50, 55, 60, 65, 70, 75, 80, 85, 90, 95, or 100 hours before the Cas effector protein is added.

In some embodiments, the inhibitor of the NHEJ pathway is added to the composition comprising a eukaryotic cell at the same time the Cas effector protein is added.

In some embodiments, the inhibitor of the NHEJ pathway is added to the composition comprising a eukaryotic cell about 0 minutes to about 30 minutes after the Cas effector protein is added, about 0 minutes to about 1 hour after the Cas effector protein is added, about 0 minutes to about 3 hours after the Cas effector protein is added, about 0 minutes to about 6 hours after the Cas effector protein is added, about 0 minutes to about 12 hours after the Cas effector protein is added, about 0 minutes to about 18 hours after the Cas effector protein is added, about 0 minutes to about 24 hours after the Cas effector protein is added, about 0 minutes to about 36 hours after the Cas effector protein is added, about 0 minutes to about 48 hours after the Cas effector protein is added, about 0 minutes to about 72 hours after the Cas effector protein is added, or about 0 minutes to about 96 hours after the Cas effector protein is added. In some embodiments, the inhibitor of the NHEJ pathway is added to the composition comprising a eukaryotic cell about 1, 2, 3, 4, 5, 6, 7, 8, 9, 10, 11, 12, 13, 14, 15, 16, 17, 18, 19, 20, 21, 22, 23, 24, 24, 25, 26, 27, 28, 29, 30, 31, 32, 33, 34, 35, 40, 45, 50, 55, 60, 65, 70, 75, 80, 85, 90, 95, or 100 hours after the Cas effector protein is added.

In some embodiments, the inhibitor of the NHEJ pathway is in the composition comprising a eukaryotic cell for about 1 to about 300 hours, about 10 to about 200 hours, about 10 to about 100 hours, about 20 to about 80 hours, about 30 to about 70 hours, or about 40 to about hours. In some embodiments, the inhibitor of the NHEJ pathway is in the composition comprising a eukaryotic cell for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, 100, 125, 150, 175, 200, 225, 250, 275, or 300 hours.

In some embodiments, the inhibitor of the NHEJ pathway is added to the composition comprising a eukaryotic cell at least 1, 2, 3, 4, 5, 6, 7, 8, 9, or 10 or more times.

In some embodiments, the inhibitor of the NHEJ pathway is added to the composition comprising a eukaryotic cell before the inhibitor of the MMEJ pathway is added to the composition comprising a eukaryotic cell after the inhibitor of the MMEJ pathway is added to the composition. In some embodiments, the inhibitor of the NHEJ pathway and the inhibitor of the MMEJ pathway are added to the composition comprising a eukaryotic cell at the same time.

In some embodiments, the inhibitor of the MMEJ pathway and the inhibitor of the NHEJ pathway are added to the composition comprising a eukaryotic cell before the Cas effector protein is added. In some embodiments, the inhibitor of the MMEJ pathway and the inhibitor of the NHEJ pathway are added to the composition comprising a eukaryotic cell after the Cas effector protein is added. In some embodiments, the inhibitor of the MMEJ pathway and the inhibitor of the NHEJ pathway are added to the composition comprising a eukaryotic cell at the same time the Cas effector protein is added. In some embodiments, the inhibitor of the MMEJ pathway is added to the composition comprising a eukaryotic cell before the Cas effector protein is added and the inhibitor of the NHEJ pathway is added after the Cas effector protein is added. In some embodiments, the inhibitor of the MMEJ pathway is added to the composition comprising a eukaryotic cell after the Cas effector protein is added and the inhibitor of the NHEJ pathway is added before the Cas effector protein is added.

All references cited herein, including patents, patent applications, papers, textbooks and the like, and the references cited therein, to the extent that they are not already, are hereby incorporated herein by reference in their entirety.

### EXAMPLES

### Crispr Examples

### Example CRISPR-1—Effect of MMEJ and NHEJ Inhibitors on Double-Strand Break Repair Pathways

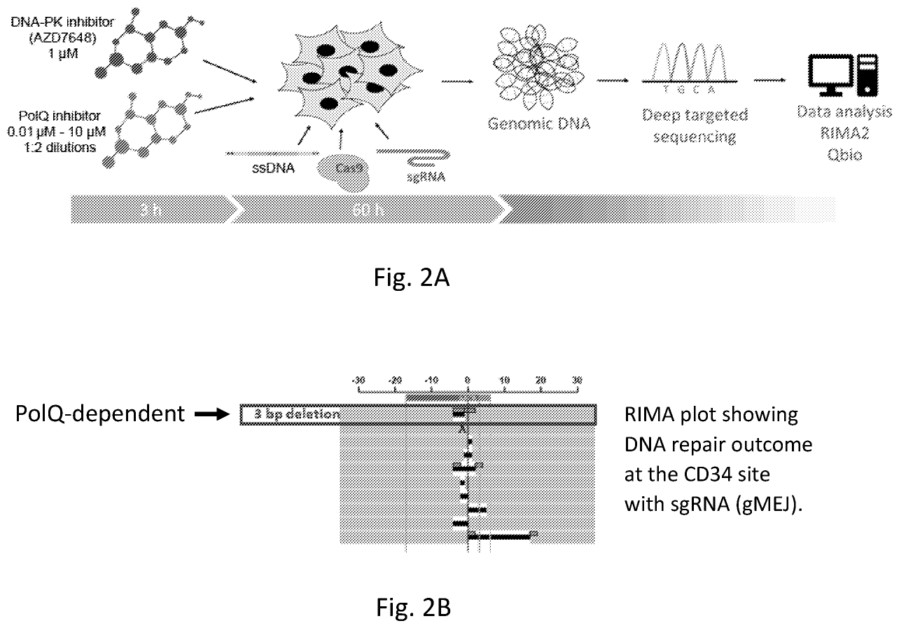

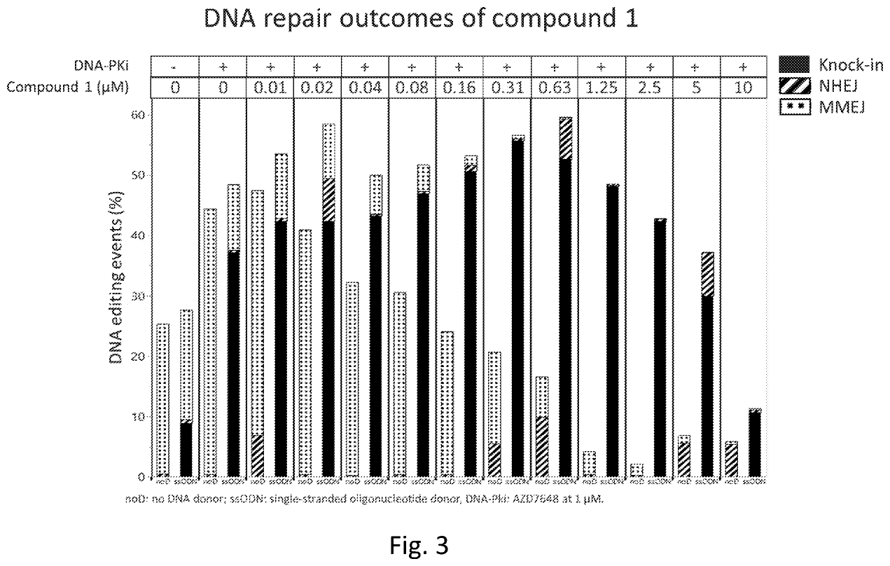

The effect of inhibitors of the MMEJ and NHEJ pathways on CRISPR-Cas-induced DNA double stranded break repair pathways was examined using the process shown schematically in FIG. 2A. Briefly, HEK293T cells were seeded into a 96-well plate 20 hours before transfection with plasmids encoding SpCas9 and a guide RNA (sgRNA) targeting CD34 in the presence and absence of single-stranded oligonucleotide donor (ssDNA). Two hours prior to transfection, the cells were submitted to inhibitor treatments, including the following conditions: a) 1 μM DNAPK inhibitor AZD7648 b) 1 μM DNAPK inhibitor AZD7648 in combination with with compound 1, which was titrated in a 1:2 dilution series, ranging between 10 μM and 0.01 μM and corresponding DMSO control. Seventy hours post-transfection, cell confluence and eGFP-based transfection efficiencies were determined with the Incucyte SX5. Genomic DNA was extracted and editing outcome was analysed through deep-targeted amplicon sequencing and RIMA2 analysis (FIG. 2A) to determine knock-in frequencies as well as mutagenic DNA repair events, such as NHEJ and MMEJ outcomes (FIG. 2B). The results of these experiments are shown in FIG. 3.

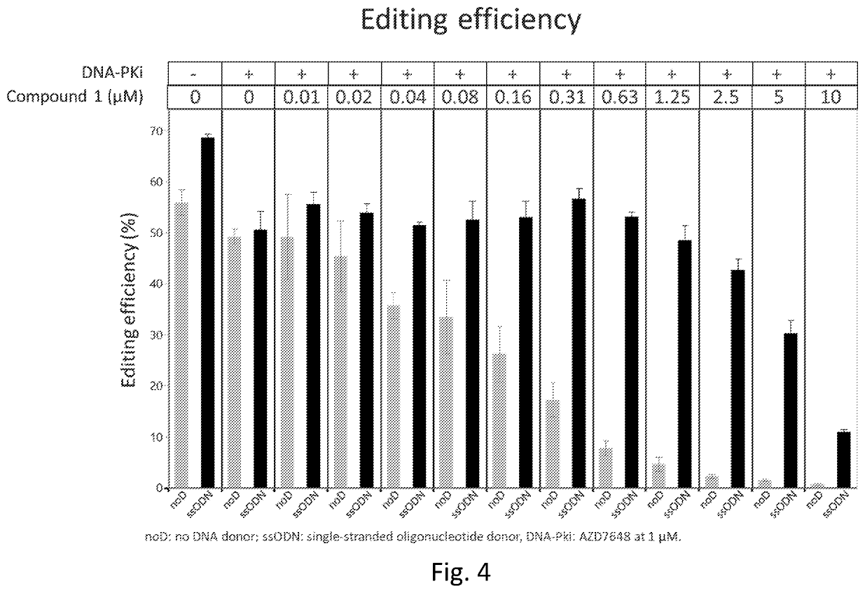

To demonstrate the effect of DNA repair inhibitors on CRISPR/Cas editing efficiency, HEK293T cells were treated with the DNA-PK inhibitor AZD7648 (1 mM) alone and in combination with the Pol Q inhibitor, Compound 1, at indicated concentrations, followed by CRISPR/Cas9-mediated gene targeting. Results are shown in FIG. 4. These studies show that inhibition of NHEJ and/or MMEJ pathways in combination with CRISPR/Cas-gene targeting results in DNA double strand break repair by the more precise HDR pathway, and minimizes the contribution from the more error-prone MMEJ and NHEJ pathways.

### Example CRISPR-2—Effect of MMEJ and NHEJ Inhibitors on CRISPR/Cas-Mediated Knock-In Efficiency

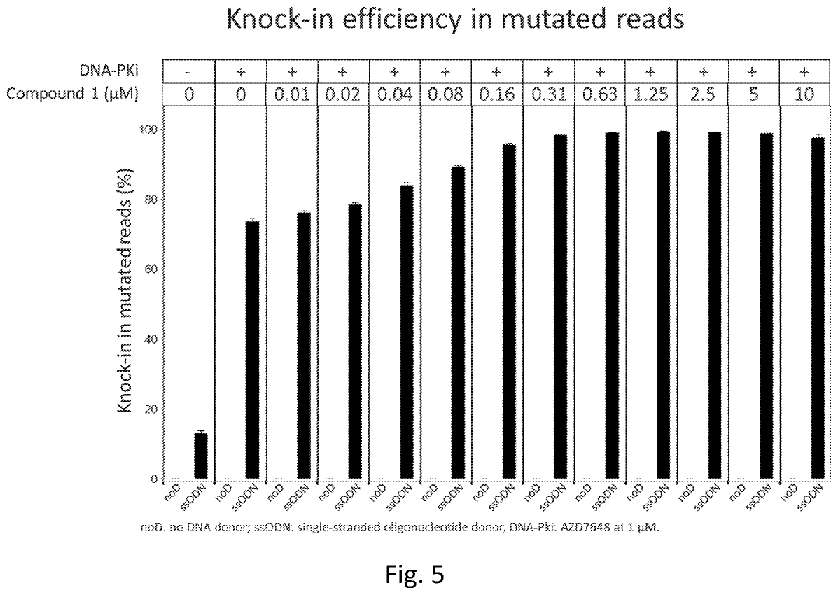

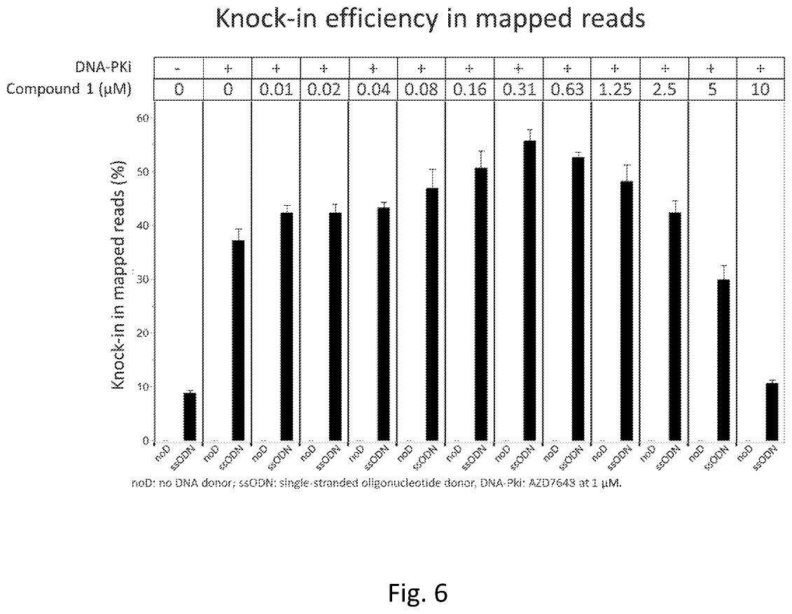

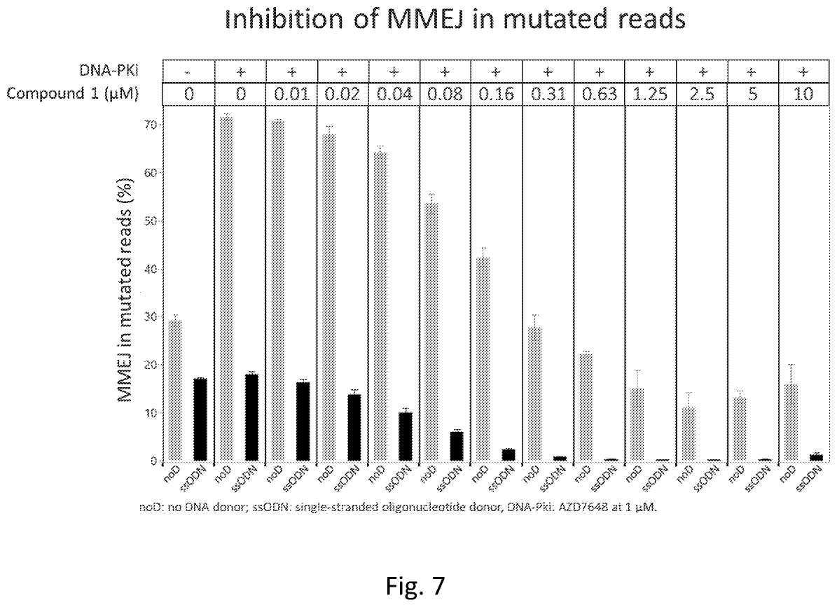

The effect of NHEJ and MMEJ inhibitors on the CRISPR/Cas-mediated knock-in efficiency was determined in both mutated and mapped reads. Briefly, HEK293T cells were cultured and transfected, and then treated with an NHEJ inhibitor (AZD7648) alone and in combination with an MMEJ inhibitor (Compound 1) at various concentrations following the protocol described in Example CRISPR-1, followed by isolation of genomic DNA and subsequent analysis of knock-in efficiency. Results are shown in FIG. 5 (mutated sequencing reads)( ) and FIG. 7 (mapped sequencing reads).

### Example CRISPR-3—Effect of MMEJ Inhibition in Mutated and Mapped Sequencing Reads

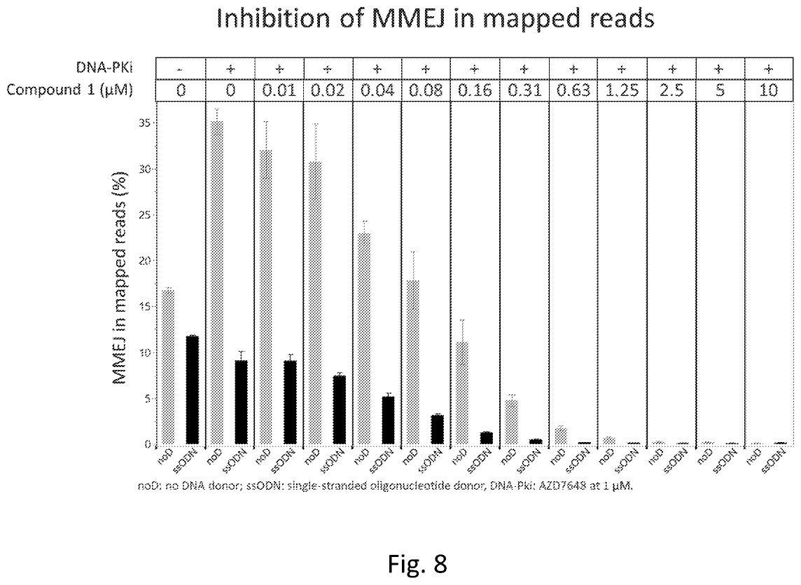

HEK293T cells were cultured, transfected, and treated with the DNA-PK inhibitor AZD7648 (1 mM) alone and in combination with the Pol Q inhibitor Compound 1 at indicated concentrations, followed by CRISPR/Cas9-mediated gene knock-in. The effect of MMEJ pathway inhibition was assessed in mutated and mapped sequencing reads. Treatment of CRISPR/Cas-edited cells with MMEJ inhibitors are shown in FIG. 7 (MMEJ-mutated reads) and FIG. 8 (MMEJ-mapped reads).

### Example CRISPR-4—Effect of the Inhibition of the NHEJ and MMEJ Pathways on Cell Confluency and Transfection Efficiency in CRISPR/Cas-Transfected Cells

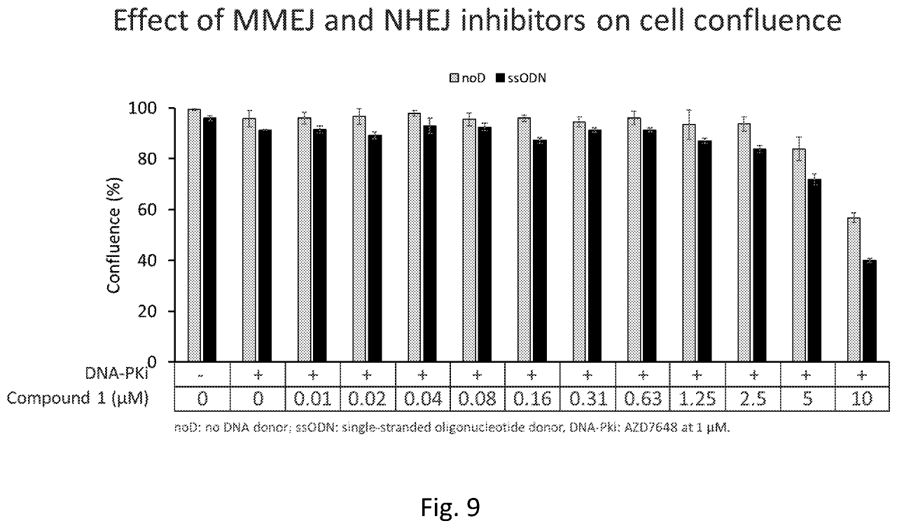

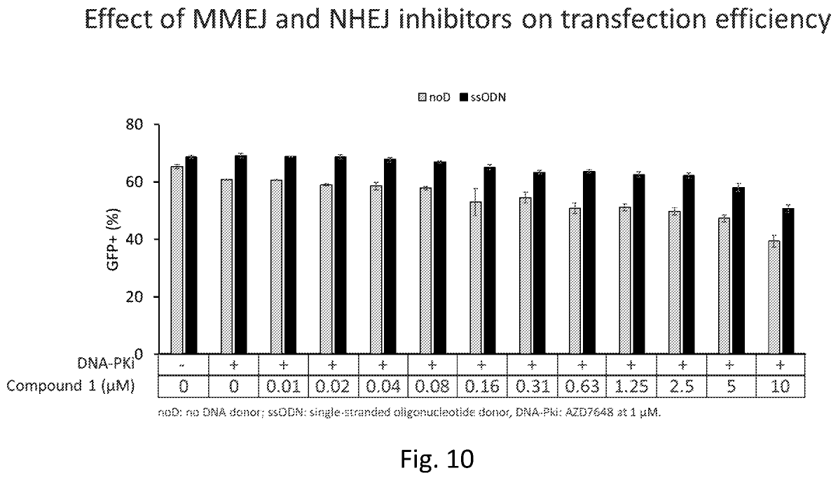

HEK293T cells were cultured, transfected, and treated with NHEJ and MMEJ inhibitors as described in Example CRISPR-1. Cell confluency and transfection efficiency was assessed in transfected cells treated with NHEJ and MMEJ inhibitors. Results are shown in FIG. 9 (cell confluency), and FIG. 10 (transfection efficiency).

### Example CRISPR-5—Effect of NHEJ and MMEJ Inhibitors on Double-Strand Break Repair Pathways in Induced Pluripotent Stem Cells (iPSCs)

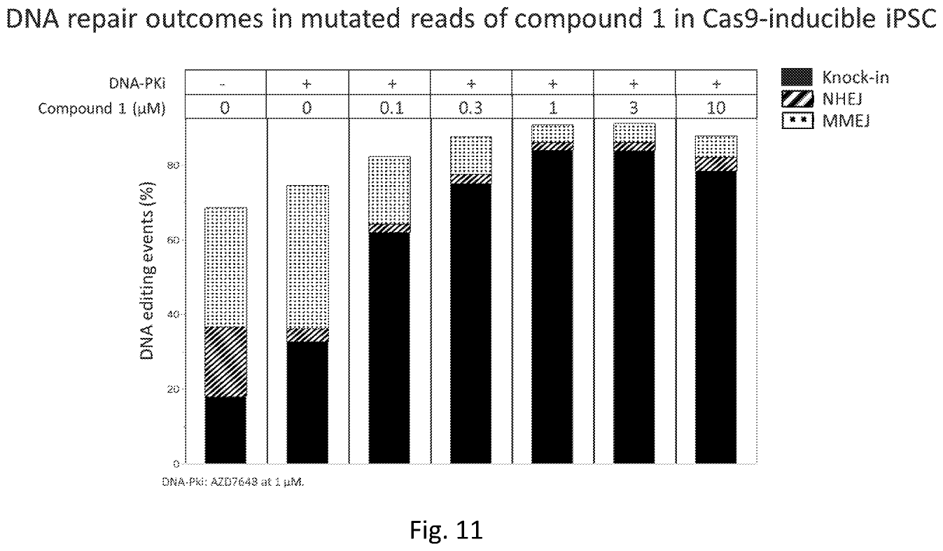

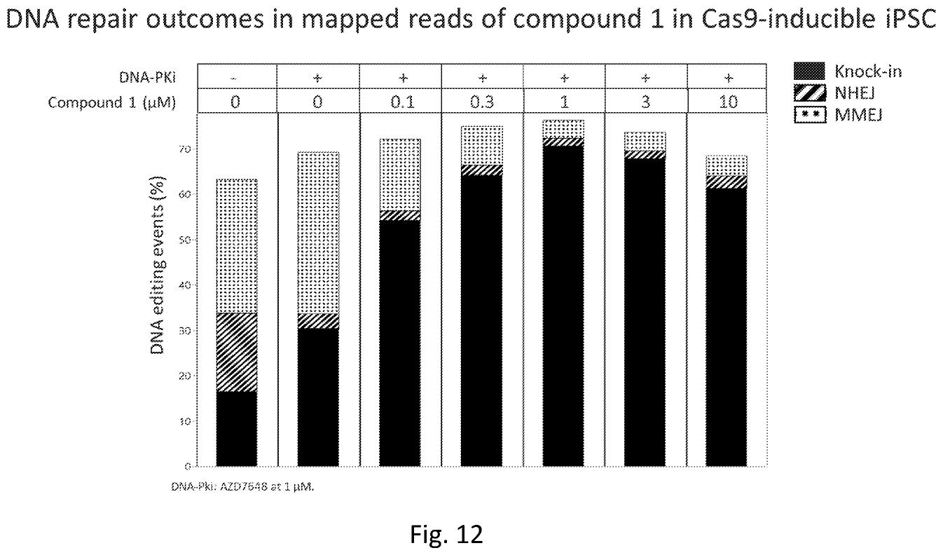

The effect of NHEJ and/or MMEJ pathway inhibition on CRISPR-Cas-induced DNA double stranded break repair pathways in iPSCs was examined. Briefly, expression of Cas9 in iPSCs was induced with 100 ng/mL doxycycline 24 hours prior to reverse transfection with a sgRNA targeting the CD34 site (gMEJ) and a single-stranded oligonucleotide donor (ssDNA). Immediately after transfections, cells were submitted to inhibitor treatments, including the following conditions: a) 1 μM DNAPK inhibitor AZD7648 b) 1 μM DNAPK inhibitor AZD7648 in combination with with Compound 1, which was titrated in a 1:3 dilution series, ranging between 10 μM and 0.1 μM and corresponding DMSO control. Seventy-two hours post-transfection, the percentage of double-stranded break repair by the HDR, NHEJ, and MMEJ pathways was determined as discussed in Example CRISPR-1. The results of these experiments are shown in FIG. 11 (mapped reads) and FIG. 12 (mutated reads).

### Example CRISPR-6—Effect of NHEJ and MMEJ Inhibitors on Single-Strand Template Repair (SSTR) Gene Insertion in iPSCs

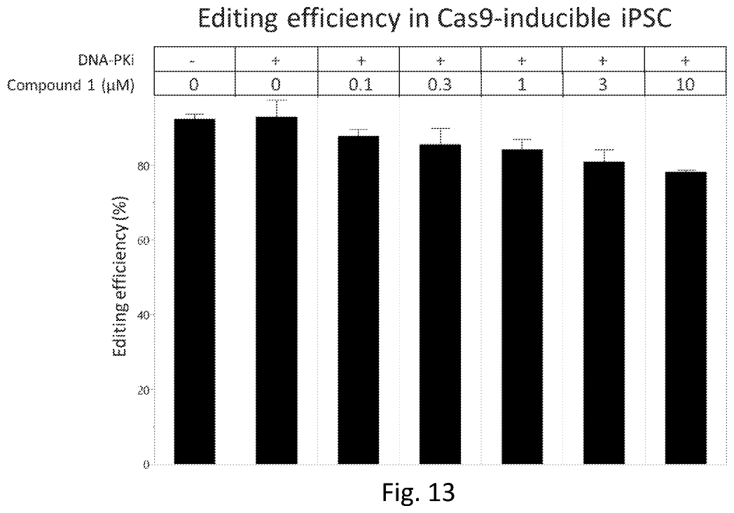

The effect of NHEJ and MMEJ pathway inhibition on editing efficiency in Cas9-inducible iPSCs was investigated. Briefly, Cas9-inducible iPSCs were cultured and transfected with sgRNA and ssDNA polynucleotides as described in Example CRISPR-5. Results are shown in FIG. 13.

### Synthetic Methods

A suitable process for the preparation of compounds of the Formula (I), or a pharmaceutically acceptable salts thereof is illustrated by the following representative process variants in which, unless otherwise stated, G, Gₐ, G_{b}, Q₁, Q₂, Q₃ and R¹, R², R^{a}, R^{b}, R^{c}, R^{d}, X, Y Zₐ/Zₐₐ, Z_{b}/Z_{bb}, Z_{c}/Z_{cc} have any of the meanings defined hereinbefore. Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

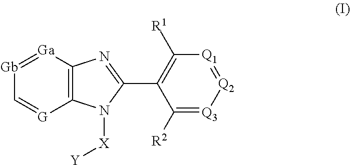

Compounds of Formula (I) may be made by, for example:
a) 1. by reaction of another compound of formula (I) where Q¹, Q² or Q³ is C—OH with a primary or secondary alcohol under conditions known in the art as suitable for Mitsunobu reaction (e.g. Synthetic Example 1);
or by reaction with a primary or secondary halide under typical conditions for nucleophilic substitution, e.g. a suitable solvent such as DMA or DMF in the presence of a suitable base, for example potassium carbonate or cesium carbonate at a suitable temperature (0-120° C.) with or without a protecting group for other functionalities.

2. by reaction of another compound of formula (I) where Q¹, Q² or Q³ is C-LG, LG being a leaving group such as halogen, with an suitable amine (e.g. Synthetic Example 19) under conditions known in the art, optionally catalysed by metal complexes such as palladium catalysts suitable for Buchwald-Hartwig amination reactions.

3. by reaction of another compound of formula (I) where Q¹, Q² or Q³ is C-LG, LG being a leaving group such as halogen, with a suitable alcohol (e.g. Synthetic Example 20) under conditions known in the art (e.g. reaction in the presence of a strong base such sodium hydride to form the alkoxide), optionally catalysed by metal complexes such as palladium catalysts (e.g. RockPhos Pd G3) suitable for ether formation reactions.

More generally, a compound of Formula (I) can be made from a compound of Formula (I) (e.g. amide coupling in Synthetic Example N1, reductive amination in Synthetic Example N6).

Compound of formula (I) where Q¹, Q² or Q³ is C—OH or where Q¹, Q² or Q³ is C-LG can be made by methods illustrated thereafter
b) When Gₐ is CR⁵ and R⁵ is

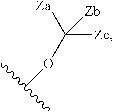

by reaction of another compound of formula (II) with a compound of formula (III), where LG is a leaving group known to the art, for example halide such as F, Cl or Br, or trifluoromethanesulfonate (triflate). Conditions for the reaction may use a suitable solvent (for example THF) in the presence of a suitable base (for example sodium hydride or LHMDS) and a suitable temperature (such as from 0° C. to ambient temperature), with or without a protecting group for other functionalities.

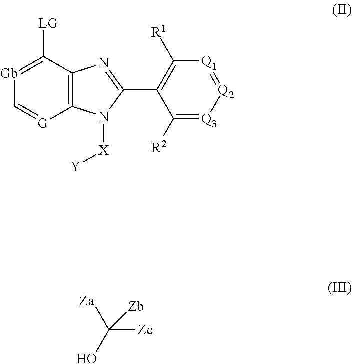

Compound of formula (II) can be made by reaction of compound of formula (IV) with compound of formula (V). Conditions for the reaction involved a one-step procedure as described in paragraph (d). The reaction can be converted in a two-step procedure with the isolation of intermediate compound of formula (VI). Conditions for the reaction are described in paragraph (d).

Alternatively, compound of formula (II) can be made by reaction of compound of formula (IVa) with compound of formula (V). Conditions for the reaction are described in paragraph (d).

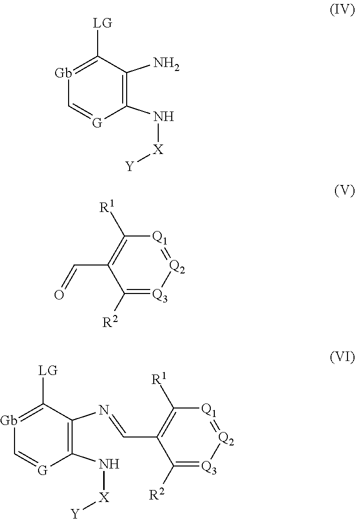

Alternatively, compound of formula (IV) can be made by reaction of formulae (IVa) by reduction of the nitro group to the amino group, as described in paragraph (d)

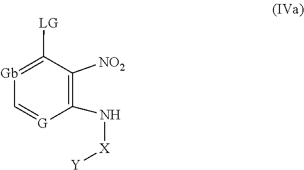

When X═CH₂, compound of formula (IVa) can be made from reaction between compound of formula (VII) and compound of formula (VIIIa) under conditions known in the art as suitable for reductive amination.

Alternatively compound of formula (IVa) can be made from reaction between compound of formula (VIIa) and compound of formula (VIII). Conditions for the reaction may use an inert solvent (for example DMF) in the presence of a base (such as triethylamine) and a suitable temperature (e.g. room temperature).

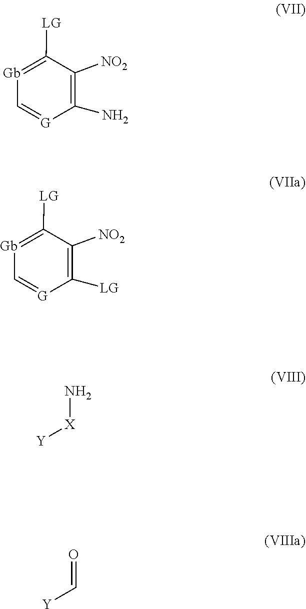

Alternatively compounds (II) can be obtained from reaction between compound of formula (IIa) and compound of formula (VIIIb) under conditions known in the art as suitable for nucleophilic substitution or from reaction between compound of formula (IIa) and compound of formula (VIIIc) under conditions known in the art as suitable for Mitsunobu reactions.

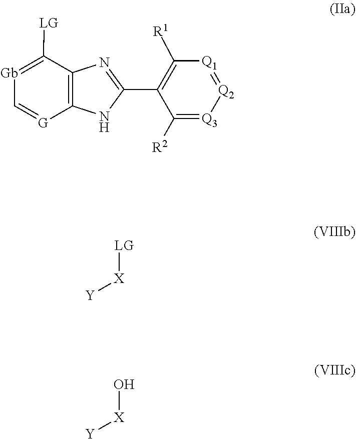

c) When G_{b} is CR⁵ and R⁵ is

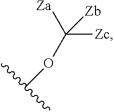

by reaction of another compound of formula (IX) with a compound of formula (X) under conditions known in the art as suitable for Mitsunobu reaction,
or by reaction nucleophilic substitution reaction of another compound of formula (IX) with a compound of formula (Xa), where LG is a leaving group known to the art, for example halide such as Cl, Br or I). Conditions for the nucleophilic substitution reaction may use a suitable solvent (for example acetonitrile, DMF or DMA) in the presence of a suitable base (for example potassium carbonate) and a suitable temperature (between 0-120° C.) with or without a protecting group for other functionalities.

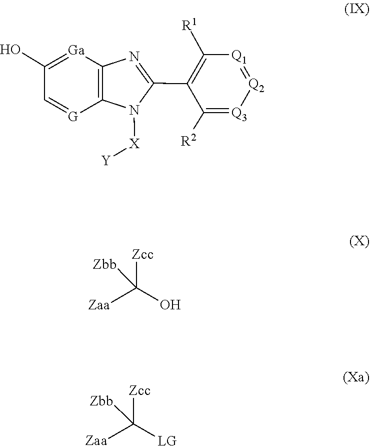

Compound of formula (IX) can be made by reaction of compound of formula (XI) with compound of formula (V), with or without a protecting group for the hydroxy group.

Compound of formula (IX) can also be made by reaction of compound of formula (XIa) with compound of formula (V), with or without a protecting group for the hydroxy group.

Alternatively compound of formula (XI) can be made from compound of formula (XIa) by reduction of the nitro group to the amino group.

Conditions for the above reactions are illustrated in paragraph (d).

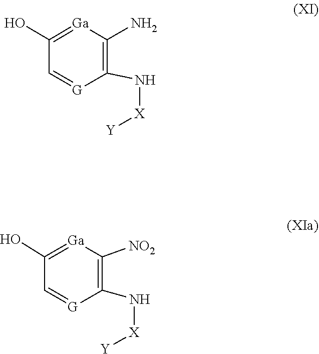

Compound of formula (XIa) can be made by reaction of compound of formula (XII) with compound of formula (VIII) where LG is a leaving group known to the art, for example halide (such as F or C1) or trifluoromethanesulfonate (triflate); with or without a protecting group for the hydroxy group.

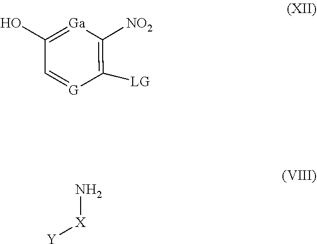

d) by reaction of compound of formula (XIII) with compound of formula (V). Conditions for the reaction involved one step procedure and may use a suitable solvent (for example EtOH, isopropanol, dioxane or DMSO) and a suitable temperature (60-120° C.), optionally in the presence of a mild oxidant (such as iron(III) chloride and/or atmospheric oxygen) and/or an acid (e.g. p-toluenesulfonic acid, acetic acid) and/or a catalyst (e.g. copper(II) acetate in Synthetic Example 8). The reaction can be converted in a two-step procedure with the isolation of intermediate compound of formula (XIV), where a mild oxidant (e.g. iron(III) chloride and/or oxygen) is added for the second step.

Alternatively by reaction of compound of formula (XIIIa) with compound of formula (V) for the reaction may use a suitable solvent (for example NMP and water) in the presence of a mild reducing agent such as sodium dithionate (also known as sodium hydrosulfite) at a suitable temperature (e.g. 80-120° C.).

Alternatively compound of formula (XIII) can be made from compound of formula (XIIIa) by reduction of the nitro group to the amino group (e.g. in the presence of iron with a suitable solvent such as ethanol)

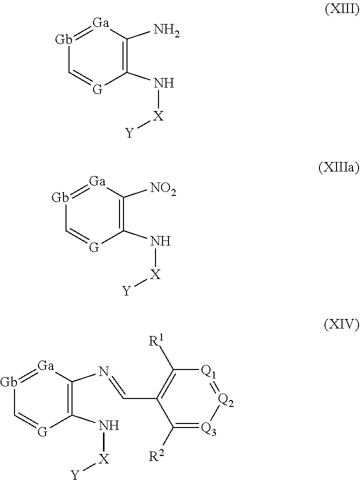

Compound of formula (XIIIa) can be made by reaction of compound of formula (XV) and another compound of formula (VIII) where LG is a leaving group known to the art, for example halide (such as F or Cl) or trifluoromethanesulfonate (triflate) or methanesulfonyl.

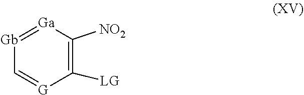

When Gₐ is CR⁵ and R⁵ is

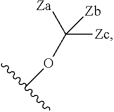

compound of formula (XIIIa) can be made by reaction of compound of formula (IVa) with compound of formula (III). Conditions for the reaction may use a suitable solvent (for example THF) in the presence of a suitable base (for example sodium hydride or LHMDS) and a suitable temperature (such around ambient temperature) with or without a protecting group for other functionalities

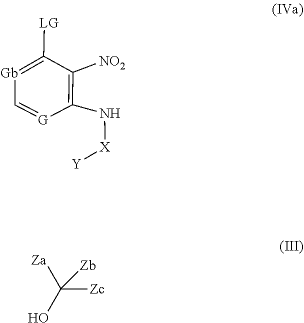

When Gₐ is CR⁵ and R⁵ is

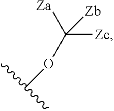

compound of formula (XIIIa) can also be made by reaction of another compound of formula (XVI) with a compound of formula (III) under conditions known in the art as suitable for Mitsunobu reaction,
or by reaction (nucleophilic substitution) of another compound of formula (XVI) with a compound of formula (IIIa), where LG is a leaving group known to the art, for example halide such as Cl, Br or I). Conditions for the nucleophilic substitution reaction may use a suitable solvent (for example acetonitrile, DMF or DMA) in the presence of a suitable base (for example potassium carbonate) and a suitable temperature (between 0-120° C.) with or without a protecting group for other functionalities

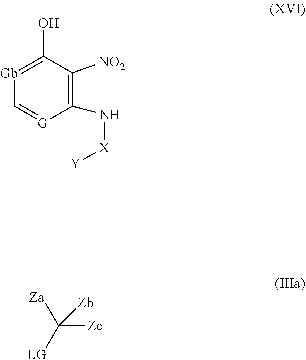

Compound of formula (XVI) can be made by reaction of another compound of formula (XVII) and another compound of formula (VIII) with or without a protecting group for the hydroxyl and other functionalities

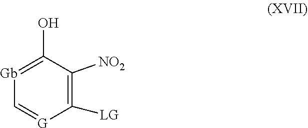

(e) by reaction of compound of formula (XVIII), when LG is a leaving group known to the art for example halide such as Cl, Br or I, with compound of formula (XIX) when FG is a functional group suitable for cross-couplings reactions (e.g. Suzuki reaction) such as a boronate ester or boronic acid. Conditions of the reaction are illustrated in Synthetic Example 5.

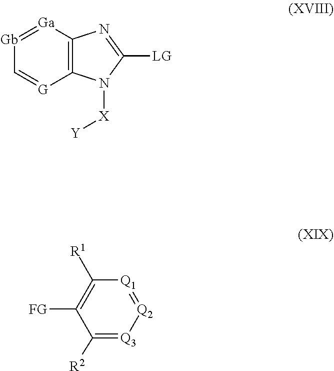

Compound of formula (XVIII) can be made from compound of formula (XX), for example by bromination (when LG is Br) as described in Synthetic Example 5.

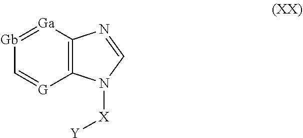

(f) from reaction between compound of formula (XXI) and compound of formula (VIIIb) under conditions known in the art as suitable for nucleophilic substitution (e.g. Synthetic Example 23).
or from reaction between compound of formula (XXI) and compound of formula (VIIIc) under conditions known in the art as suitable for Mitsunobu reactions.

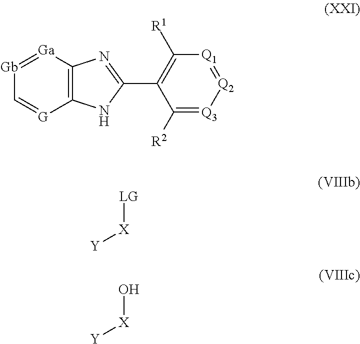

It is understood that compound of formula (XXI), by analogy with compound of formula (I), may be made by reaction already illustrated in the paragraphs (a) to (e).

### General Experimental Conditions and Abbreviations

The compounds described herein are further illustrated in the following Synthetic Examples. The compounds were named using Chemdraw version 20.0.2.51. These Examples are given by way of illustration only and are non-limiting. In general:

Reagents and solvents (all anhydrous HPLC-grade) were obtained from commercial suppliers and used without any further purification unless otherwise stated. All reagents were weighed and handled in air unless otherwise stated. Brine refers to a saturated solution of NaCl. Concentration under reduced pressure refers to the use of a rotary evaporator.

Operations were carried out at ambient temperature, i.e. in the range 17 to 25° C. and under an atmosphere of an inert gas such as nitrogen unless otherwise stated.

Evaporations were carried out by rotary evaporation under reduced pressure utilising a warm or hot water bath or utilising Genevac equipment or Biotage v10 evaporator in vacuo and work up procedures were carried out after removal of residual solids by filtration.

Flash chromatographic purifications were performed on an automated Teledyne Isco CombiFlash® Rf, Teledyne Isco CombiFlash® Companion® or CHEETAHφ MP200 system with integrated UV detection using prepacked silica gel columns (40-60 μm) or C18 spherical (20-35 μm) using the chromatographic conditions as detailed in corresponding experimental data.

Preparative reverse phase HPLC was performed on an Agilent 1290 Infinity II Preparative system equipped with a SQ MS detector (Multimode ESI/APCI source), with a Waters CSH C18 OBD column (5 microns silica, 30 mm diameter, 100 mm length); Waters MassLynx system with integrated MS detection, with a XBridge or Xselect CSH Prep C18 OBD column (5 μm silica, 30 mm diameter, 150 mm length); Gilson GX-281 with integrated UV detection, with either XBridge (10 μm, 19 mm diameter, 150 mm length) or Sunfire C18 columns (10 m, 19 mm diameter, 250 mm length) using decreasingly polar mixtures of water (containing 0.1-0.3% aqueous ammonium), water (containing 0.05% aqueous ammonia and 10 mmol NH₄HCO₃), water (containing 0.1% formic acid) or water (containing 0.05% TFA) and acetonitrile or methanol as eluents.

Preparative SFC purification was performed on either a Sepiatec P100 SFC system or Waters Prep 100 SFC system equipped with QDa MS detector, using the chromatographic conditions as detailed in corresponding experimental data.

Preparative chiral HPLC was performed with a Gilson GX-281 system with integrated UV detection and equipped with one of Chiralpak AS, AD, Chiralcel OD,OJ Chiralpak IA,IB,IC,ID,IE,IF,IG,IH columns (Daicel Chemical Industries, Ltd.) (R,R)-Whelk-O1, (S,S)-Whelk-O1 columns (Regis technologies, Inc.) CHIRAL Cellulose-SB, SC, SA columns (YMC Co., Ltd.) at different column size (250×20 mm, 250×30 mm) with noted percentage of either ethanol in hexane (% Et/Hex) or isopropanol in hexane (% IPA/Hex) as isocratic solvent systems.

Yields, where present, are not necessarily the maximum attainable.

In general, the structures of end-products of the Formula I were confirmed by nuclear magnetic resonance (NMR) spectroscopy; ¹H-NMR chemical shift values were measured on the delta scale and are quoted in ppm with measurement against TMS or residual solvent peaks as internal standards; proton magnetic resonance spectra were determined using a Bruker Avance 500 spectrometer at a proton frequency of 500 MHz, Bruker Avance 400, Bruker Avance III HD or Bruker Avance Neo spectrometers at a proton frequency of 400 MHz or Bruker Avance III, Avance III HD or Avance III NEO spectrometers at a proton frequency of 300 MHz; measurements were taken at ambient temperature unless otherwise specified; the following abbreviations have been used: s, singlet; d, doublet; t, triplet; q, quartet; m, multiplet; dd, doublet of doublets; ddd, doublet of doublet of doublet; dt, doublet of triplets; br s, broad signal; hept, heptet.

In general, end products of the Formula I were also characterized by mass spectrometry following liquid chromatography (LCMS or UPLC); reverse-phase C18 silica was used with a flow rate of 1 mL/min and detection was by Electrospray Mass Spectrometry and by UV/vis absorbance recording a wavelength range of 220-320 nm. Analytical UPLC was performed using a Waters Acquity UPLC CSH C18 column with dimensions 2.1×50 mm and particle size 1.7 micron) Gradient analysis was employed using decreasingly polar mixtures as eluent, for example decreasingly polar mixtures of water (containing 0.1% v/v formic acid or 0.3% ammonia v/v) as solvent A and acetonitrile as solvent B. A typical 1.7 minute analytical UPLC method would employ a solvent gradient over 1.3 min, at 1 mL/min, from a 97:3 mixture of solvents A and B respectively to a 3:97 mixture of solvents A and B. Also, LCMS was performed using a Shimadzu LCMS-2020 with electrospray ionization in positive ion detection mode with 20ADXR pump, SIL-20ACXR autosampler, CTO-20AC column oven, M20A PDA Detector and LCMS 2020 MS detector. LC was run in two set ups: 1) Halo C18 column (2.0 μm 3.0×30 mm) in combination with a gradient (5-100% B in 1.2 minutes) of water and formic acid-FA (0.1%) (A) and CH₃CN and FA (0.1%) (B) at a flow rate of 1.5 mL/min; 2) Poroshell HPH C18 column (2.7 μm 3.0×50 mm) in combination with a gradient (5-95% B in 2 minutes) of aqueous 46 mM ammonium carbonate/ammonia buffer at pH 10 (A) and MeCN (B) at a flow rate of 1.2 mL/min; 3) Halo C18 column (2.0 μm 3.0×30 mm) in combination with a gradient (5-95% B in 2 minutes) of water and TFA (0.05%) (A) and CH₃CN and TFA (0.05%) at a flow rate of 1.5 mL/min (B). The Column Oven (CTO-20AC) temperature was 40.0° C. The injection volume was 1 μL. PDA (SPD-M20A) detection was in the range 190-400 nm. The MS detector, which was configured with electrospray ionization as ionizable source; Acquisition mode: Scan; Nebulizing Gas Flow:1.5 L/min; Drying Gas Flow:15 L/min; Detector Voltage: Tuning Voltage±0.2 kv; DL Temperature: 250 oC; Heat Block Temperature: 250 oC; Scan Range: 90.00-900.00 m/z. It is understood that, unless otherwise specified, the reported molecular ion corresponds to the [M+H]+, rounded to the lower unit. Typically, unless otherwise specified; for molecules with multiple isotopic patterns (e.g. ³⁵Cl, ⁷⁹Br, ¹²C) only the lower most common isotope is reported.

Ion exchange purification was generally performed using a SCX-2 (Biotage, Propylsulfonic acid functionalized silica. Manufactured using a trifunctional silane. Non end-capped) cartridge.

Intermediate purity was assessed by thin layer chromatographic, mass spectral, HPLC (high performance liquid chromatography) and/or NMR analysis.

The following abbreviations have been used

| | |
|---|---|
| Ac₂O | acetic anhydride |
| AcOH | acetic acid |
| aq. | aqueous |
| BINAP | 2,2′-bis(diphenylphosphino)- |
| | 1,1′-binaphthalene |
| BOC | tert-butyloxycarbonyl |
| Cbz | benzyloxycarbonyl |
| DCM | dichloromethane |
| DEA | diethylamine |
| DIAD | diisopropyl azodicarboxylate |
| DIEA | diisopropylethylamine |
| DMA | N,N-dimethylacetamide |
| DMF | N,N-dimethylformamide |
| DMSO | dimethyl sulphoxide |
| Et₂O | diethyl ether |
| EtOAc | ethyl acetate |
| EtOH | ethanol |
| h | hour(s) |
| HATU | 1-[Bis(dimethylamino)methylene]- |
| | 1H-1,2,3-triazolo[4,5- |
| | b]pyridinium 3-oxid hexafluorophosphate |
| HOBt | 1-hydroxybenzotriazole |
| HPLC | high performance liquid chromatography |
| IPA or iPrOH | isopropanol |
| LCMS | liquid chromatography-mass spectrometry |
| LHMDS | lithium bis(trimethylsilyl)amide |
| MeCN | acetonitrile |
| Mel | methyl iodide |
| MeOH | methanol |
| min | minute(s) |
| Ms₂O | methanesulfonic anhydride |
| MTBE | methyl tert-butyl ether |
| NMP | 1-methyl-2-pyrrolidone |
| m/z | mass to charge ratio |
| NMR | nuclear magnetic resonance |
| PDA | photo-diode array |
| Pd₂(dba)₃ | tris(dibenzylideneacetone)dipalladium(0) |
| PdCl₂(dppf) | [1,1′-bis(diphenylphosphino)ferrocene] |
| | dichloropalladium(II) |
| PdCl₂(PPh₃)₂ | bis(triphenylphosphine)palladium(II) |
| | dichloride |
| Pd(PPh₃)₄ | palladium-tetrakis(triphenylphosphine) |
| PPh₃ | triphenyl phosphine |
| RockPhos | [(2-di-tert-butylphosphino-3-methoxy- |
| Pd G3 | 6-methyl-2′,4′,6′-triisopropyl-1,1′- |
| | biphenyl)-2-(2-aminobiphenyl)] |
| | palladium(II) methanesulfonate |
| rac | racemic |
| rac-BINAP | see CAS Number: 2151915-22-7 |
| Pd | |
| G3 | |
| rt/RT | room temperature |
| scCO₂ | supercritical carbon dioxide |
| SCX | strong cation exchanger |
| SFC | supercritical fluid chromatography |
| T3P | Propylphosphonic anhydride |
| TBAF | Tetra-N-butylammonium fluoride |
| TBDPS | tert-butyldiphenylsilyl |
| TEA | triethylamine |
| tert | tertiary |
| TFA | trifluoroacetic acid |
| THF | tetrahydrofuran |
| TLC | thin layer chromatography |
| TMS | tetramethylsilane |
| UPLC | ultra performance liquid chromatography |
| UV | ultraviolet |

### Synthetic Example 1

### 9-Benzyl-6-isopropoxy-8-(2-methyl-4-(2-(4-methylpiperazin-1-yl)ethoxy)phenyl)-9H-purine

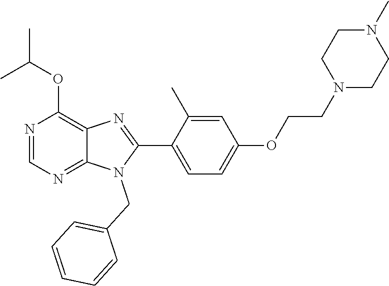 [SMILES: Cc1cc(OCCN2CCN(C)CC2)ccc1-c1nc2c(OC(C)C)ncnc2n1Cc1ccccc1]

DIAD (0.260 mL, 1.34 mmol) was added dropwise to 4-(9-benzyl-6-isopropoxy-9H-purin-8-yl)-3-methylphenol (200 mg, 0.53 mmol), 2-(4-methylpiperazin-1-yl)ethan-1-ol (116 mg, 0.80 mmol) and PPh₃ (392 mg, 1.50 mmol) in THF (20 mL) at 0° C. under nitrogen. The resulting mixture was stirred at rt for 16 hours. The residue was purified by preparative TLC (EtOAc) and then further purified by preparative HPLC (XBridge Shield RP18 OBD column, 5 μm silica, 30 mm diameter, 150 mm length), using decreasingly polar mixtures of water (containing 0.05% aq. NH₃) and MeCN as eluents. Fractions containing the desired compound were evaporated to dryness to afford 9-benzyl-6-isopropoxy-8-(2-methyl-4-(2-(4-methylpiperazin-1-yl)ethoxy)phenyl)-9H-purine (76 mg, 28%) as a yellow oil. ¹H NMR (300 MHz, DMSO-d₆): 1.42 (6H, d), 1.95 (3H, s), 2.16 (3H, s), 2.33 (8H, m), 2.70 (2H, t), 4.13 (2H, t), 5.26 (2H, s), 5.62 (1H, p), 6.84-6.94 (4H, m), 7.18-7.31 (4H, m), 8.55 (1H, s). m/z: ES+[M+H]+ 501.

4-(9-Benzyl-6-isopropoxy-9H-purin-8-yl)-3-methylphenol used as starting material was made as follows:

### N4-Benzyl-6-chloropyrimidine-4,5-diamine

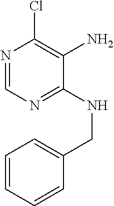 [SMILES: Nc1c(Cl)ncnc1NCc1ccccc1]

A mixture of phenylmethanamine (47.0 g, 439 mmol) and triethylamine (74.0 g, 732 mmol) was added dropwise to 4,6-dichloropyrimidin-5-amine (60 g, 366 mmol) in DMA (400 mL) at 100° C. over a period of 20 minutes under air. The resulting solution was stirred at 100° C. for 8 hours. The reaction mixture was then poured into water (1000 mL) under stirring. The resulting precipitate was collected by filtration, washed with water (500 mL) and dried at 60° C. to afford N4-benzyl-6-chloropyrimidine-4,5-diamine (60.0 g, 70%) as a pale yellow solid.

¹H NMR (300 MHz, DMSO-d₆): 4.65 (2H, d), 5.11 (2H, s), 7.19-7.29 (1H, m), 7.29-7.45 (5H, m), 7.76 (1H, s). m/z: ES+[M+H]+ 235.

### 4-(9-Benzyl-6-chloro-9H-purin-8-yl)-3-methylphenol

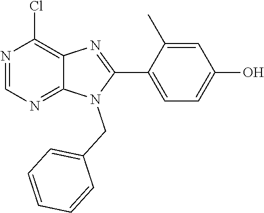 [SMILES: Cc1cc(O)ccc1-c1nc2c(Cl)ncnc2n1Cc1ccccc1]

4-Hydroxy-2-methylbenzaldehyde (580 mg, 4.26 mmol) was added to N4-benzyl-6-chloropyrimidine-4,5-diamine (500 mg, 2.13 mmol) and iron(III) chloride supported on silica gel (6.91 g, 6.39 mmol) in 1,4-dioxane (40 mL). The resulting mixture was stirred at 100° C. for 3 days. The solvent was removed under reduced pressure. The filtrate was collected by filtration, the precipitate was washed with EtOAc (100 mL) and dried under vacuum to afford a crude product. The crude product was purified by flash C18-flash chromatography, elution gradient 5 to 80% MeCN in water (containing 0.05% NH₄HCO₃). Pure fractions were evaporated to dryness to afford 4-(9-benzyl-6-chloro-9H-purin-8-yl)-3-methylphenol (230 mg, 31%) as a yellow solid. ¹H NMR (400 MHz, DMSO-d₆): 1.94 (3H, s), 5.34 (2H, s), 6.70-6.75 (2H, m), 6.87-6.92 (2H, m), 7.19-7.26 (4H, m), 8.81 (1H, s), 9.94 (1H, s). m/z: ES+[M+H]+ 351.

### 4-(9-Benzyl-6-isopropoxy-9H-purin-8-yl)-3-methylphenol

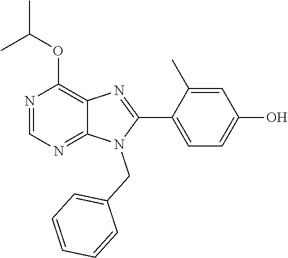 [SMILES: Cc1cc(O)ccc1-c1nc2c(OC(C)C)ncnc2n1Cc1ccccc1]

NaH (50.2 mg, 1.25 mmol) was added portionwise to 4-(9-benzyl-6-chloro-9H-purin-8-yl)-3-methylphenol (220 mg, 0.63 mmol) and IPA (0.097 mL, 1.25 mmol) in THF (10 mL). The resulting mixture was stirred at rt for 2 hours. The reaction mixture was quenched with saturated NaHCO₃ (50 mL) and extracted with EtOAc (3×20 mL). The organic layer was dried over Na₂SO₄, filtered and evaporated to afford 4-(9-benzyl-6-isopropoxy-9H-purin-8-yl)-3-methylphenol (210 mg, 89%) as a yellow solid. ¹H NMR (300 MHz, DMSO-d₆): 1.41 (6H, d), 1.90 (3H, s), 5.26 (2H, s), 5.61 (1H, p), 6.68-6.75 (2H, m), 6.84-6.90 (2H, m), 7.13-7.25 (4H, m), 8.54 (1H, s), 9.85 (1H, s). m/z: ES+[M+H]+ 375.

### Synthetic Example 2

### 2-(2-Chloro-4-(2-(4-methylpiperazin-1-yl)ethoxy)phenyl)-1-(3-chlorobenzyl)-5-isopropoxy-1H-benzo[d]imidazole

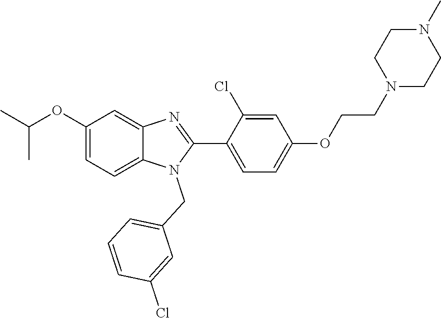 [SMILES: CC(C)Oc1ccc2c(c1)nc(-c1ccc(OCCN3CCN(C)CC3)cc1Cl)n2Cc1cccc(Cl)c1]

2-(2-Chloro-4-(2-(4-methylpiperazin-1-yl)ethoxy)phenyl)-1-(3-chlorobenzyl)-1H-benzo[d]imidazol-5-ol (125 mg, 0.24 mmol), 2-bromopropane (0.046 mL, 0.49 mmol) and potassium carbonate (169 mg, 1.22 mmol) were suspended in acetonitrile (5 mL) and sealed into a microwave tube. The reaction was heated to 100° C. for 12 hours in the microwave reactor and cooled to room temperature. The solid was removed by filtration and the filtrate evaporated to dryness. The crude product was purified by preparative HPLC (Waters CSH C18 OBD column, 5 μm silica, 30 mm diameter, 100 mm length), using decreasingly polar mixtures of water (containing 1% aqueous ammonia) and MeCN as eluents. Fractions containing the desired compound were evaporated to dryness to afford 2-(2-chloro-4-(2-(4-methylpiperazin-1-yl)ethoxy)phenyl)-1-(3-chlorobenzyl)-5-isopropoxy-1H-benzo[d]imidazole (14 mg, 10%). ¹H NMR (500 MHz, CD30D): 1.32 (3H, s), 1.33 (3H, s), 2.27 (3H, s), 2.43-2.72 (6H, m), 2.84 (2H, t), 3.30 (2H, p), 4.19 (2H, d), 4.55-4.64 (1H, m), 5.26 (2H, s), 6.86 (1H, dt), 6.93 (2H, dd), 7.01 (1H, dd), 7.15-7.23 (4H, m), 7.31 (1H, d), 7.35 (1H, d). m/z: ES+[M+H]+ 553.

2-(2-Chloro-4-(2-(4-methylpiperazin-1-yl)ethoxy)phenyl)-1-(3-chlorobenzyl)-1H-benzo[d]imidazol-5-ol used as starting material was made as follows:

### 2-(2-Chloro-4-(2-(4-methylpiperazin-1-yl)ethoxy)phenyl)-1-(3-chlorobenzyl)-5-methoxy-1H-benzo[d]imidazole

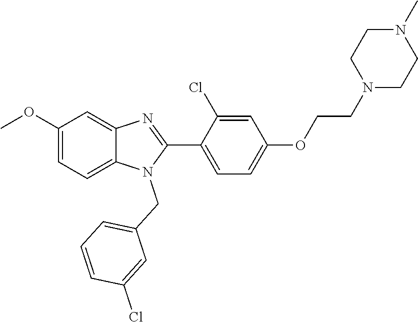

A solution of crude 2-chloro-4-(2-(4-methylpiperazin-1-yl)ethoxy)benzaldehyde (578 mg, 1.02 mmol) in NMP (3 mL) was added in one portion to a stirred suspension of N-(3-chlorobenzyl)-4-methoxy-2-nitroaniline (299 mg, 1.02 mmol, commercially available from Princeton BiolMolecular Research Inc., ACD Identifier: MFCD12564576) and sodium dithionite (628 mg, 3.07 mmol) in water (1 mL). The resulting solution was stirred at reflux for 18 hours. The reaction mixture was diluted with EtOAc (50 mL), and washed sequentially with saturated NaHCO₃ (50 mL), water (50 mL), and saturated brine (5 mL). The organic layer was dried with MgSO₄, filtered and evaporated onto silica gel (1 g). The resulting powder was purified by flash silica chromatography, elution gradient 0 to 20% MeOH in DCM with ammonia as modifier. Pure fractions were evaporated to dryness to afford 2-(2-chloro-4-(2-(4-methylpiperazin-1-yl)ethoxy)phenyl)-1-(3-chlorobenzyl)-5-methoxy-1H-benzo[d]imidazole (213 mg, 40%) as a white solid. Impure fractions were combined, concentrated in vacuo and repurified by preparative HPLC (Waters CSH C18 OBD column, 5 μm silica, 30 mm diameter, 100 mm length), using decreasingly polar mixtures of water (containing 1% aq. NH₃) and MeCN as eluents. Fractions containing the desired compound were evaporated to dryness to afford 2-(2-chloro-4-(2-(4-methylpiperazin-1-yl)ethoxy)phenyl)-1-(3-chlorobenzyl)-5-methoxy-1H-benzo[d]imidazole (92 mg, 17%) as a white solid. ¹H NMR (500 MHz, CDCl₃): 2.30 (3H, s), 2.48 (4H, s), 2.63 (4H, s), 2.84 (2H, t), 3.87 (3H, s), 4.14 (2H, t), 5.17 (2H, s), 6.82 (1H, d), 6.88 (1H, dd), 6.91 (1H, dd), 6.95 (1H, s), 7.04-7.1 (2H, m), 7.15 (1H, t), 7.18-7.22 (1H, m), 7.35 (2H, s). m/z: ES+525 [M+H]+ 525.

### 2-(2-Chloro-4-(2-(4-methylpiperazin-1-yl)ethoxy)phenyl)-1-(3-chlorobenzyl)-1H-benzo[d]imidazol-5-ol

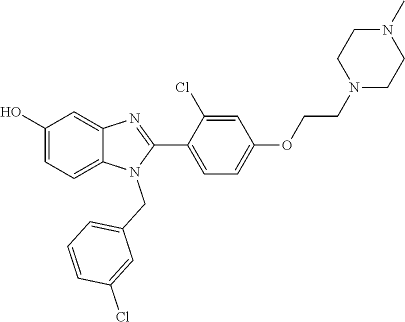 [SMILES: CN1CCN(CCOc2ccc(-c3nc4cc(O)ccc4n3Cc3cccc(Cl)c3)c(Cl)c2)CC1]

A solution of boron tribromide in dichloromethane (1.52 mL, 1.52 mmol) was added dropwise to a stirred solution of 2-(2-chloro-4-(2-(4-methylpiperazin-1-yl)ethoxy)phenyl)-1-(3-chlorobenzyl)-5-methoxy-1H-benzo[d]imidazole (0.20 g, 0.38 mmol) in anhydrous dichloromethane (1 mL) at 0° C. over a period of 2 minutes under nitrogen. The resulting suspension was stirred at room temperature for 35 minutes. The reaction mixture was quenched with 2M HCl (5 mL) and evaporated to remove the DCM. DMSO (2 mL) was added and the resulting solution was purified by flash reverse phase silica chromatography, elution gradient 5 to 95% MeCN in water with 0.1% formic acid as modifier. Pure fractions were evaporated to dryness to afford 2-(2-chloro-4-(2-(4-methylpiperazin-1-yl)ethoxy)phenyl)-1-(3-chlorobenzyl)-1H-benzo[d]imidazol-5-ol (150 mg, 77%) as a white solid. ¹H NMR (500 MHz, CDCl₃): 2.64 (3H, s), 2.81-2.96 (6H, m), 3.06 (4H, s), 4.11 (2H, t), 5.15 (2H, s), 6.83 (2H, td), 6.87 (1H, dd), 6.93 (1H, s), 7-7.06 (2H, m), 7.12-7.22 (2H, m), 7.29-7.35 (2H, m), 8.38 (1H, s). m/z: ES+[M+H]+ 511.

2-Chloro-4-(2-(4-methylpiperazin-1-yl)ethoxy)benzaldehyde used as starting material is described in Synthetic Example 4.

### Synthetic Example 3

### 3-(4-(1-Benzyl-5-isopropoxy-1H-benzo[d]imidazol-2-yl)-3-chlorophenoxy)propan-1-amine

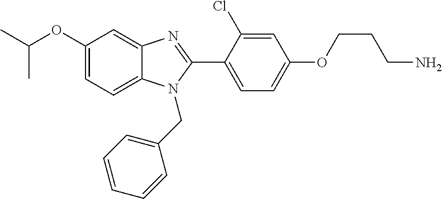 [SMILES: CC(C)Oc1ccc2c(c1)nc(-c1ccc(OCCCN)cc1Cl)n2Cc1ccccc1]

TFA (2 mL, 25.96 mmol) was added slowly to tert-butyl (3-(4-(1-benzyl-5-isopropoxy-1H-benzo[d]imidazol-2-yl)-3-chlorophenoxy)propyl)carbamate (100 mg, 0.18 mmol) in DCM (5 mL) at 0° C. The resulting mixture was stirred at rt for 2 hours. The reaction mixture was evaporated to a crude oil. The crude product was purified by preparative HPLC (XBridge Shield RP18 OBD column, 5 μm silica, 30 mm diameter, 150 mm length), using decreasingly polar mixtures of water (containing 0.05% aq. NH₃) and MeCN as eluents. Fractions containing the desired compound were evaporated to dryness to afford 3-(4-(1-benzyl-5-isopropoxy-1H-benzo[d]imidazol-2-yl)-3-chlorophenoxy)propan-1-amine (27 mg, 33%) as a yellow oil which solidified on standing. ¹H NMR (400 MHz, CDCl₃): 1.39 (6H, d), 1.97-2.07 (2H, m), 2.99 (2H, t), 4.13 (2H, t), 4.58 (1H, p), 5.23 (2H, s), 6.88 (2H, dt), 6.97-7.03 (2H, m), 7.06-7.13 (2H, m), 7.25 (3H, dd), 7.33-7.41 (2H, m). 2H not observed. m/z: ES+[M+H]+ 450.

Tert-butyl (3-(4-(1-benzyl-5-isopropoxy-1H-benzo[d]imidazol-2-yl)-3-chlorophenoxy) propyl)carbamate used as starting material was made as follows:

### N-Benzyl-4-isopropoxy-2-nitroaniline

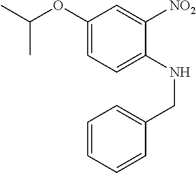 [SMILES: CC(C)Oc1ccc(NCc2ccccc2)c([N+](=O)[O-])c1]

Phenylmethanamine (2.37 g, 22.1 mmol) was added slowly to DIEA (7.02 mL, 40.2 mmol) and 1-fluoro-4-isopropoxy-2-nitrobenzene (4 g, 20.1 mmol, commercially available) in DMA (10 mL) at rt. The resulting mixture was stirred at 100° C. for 18 hours. and cooled to rt. The reaction mixture was poured into water (50 mL) and extracted with EtOAc (3×50 mL). The organic layer was washed sequentially with saturated NH₄Cl (20 mL×1), saturated NaHCO₃ (20 mL×1), and saturated brine (20 mL×1). The organic layer was dried over Na₂SO₄, filtered and evaporated to afford crude material. The residue was purified by preparative TLC (petroleum ether: EtOAc=1:6), to afford N-benzyl-4-isopropoxy-2-nitroaniline (4.50 g, 78%) as a red oil which solidified on standing. ¹H NMR (400 MHz, DMSO-d₆): 1.22 (6H, d), 4.47 (1H, hept), 4.60 (2H, d), 6.88 (1H, d), 7.18 (1H, dd), 7.25 (1H, ddd), 7.29-7.39 (4H, m), 7.51 (1H, d), 8.51 (1H, t). m/z (ES+), [M+H]+=287.

### 4-(1-Benzyl-5-isopropoxy-1H-benzo[d]imidazol-2-yl)-3-chlorophenol—Synthetic Example A1

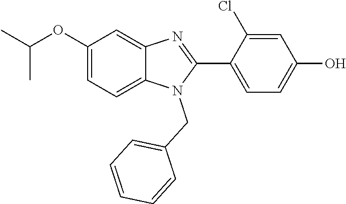 [SMILES: CC(C)Oc1ccc2c(c1)nc(-c1ccc(O)cc1Cl)n2Cc1ccccc1]

A solution of sodium dithionite (7.30 g, 41.9 mmol) in water (5.00 mL) was added dropwise to a stirred mixture of N-benzyl-4-isopropoxy-2-nitroaniline (3 g, 10.5 mmol) and 2-chloro-4-hydroxybenzaldehyde (1.80 g, 11.5 mmol) in NMP (20 mL) at rt. The resulting mixture was stirred at 100° C. for 18 hours. The reaction mixture was poured into saturated brine (75 mL) and extracted with EtOAc (3×100 mL). The organic layer was dried over Na₂SO₄, filtered and evaporated to afford a yellow gum. The crude product was purified by flash silica chromatography, elution gradient 0 to 100% petroleum ether in EtOAc. Pure fractions were evaporated to dryness to afford 4-(1-benzyl-5-isopropoxy-1H-benzo[d]imidazol-2-yl)-3-chlorophenol (2.30 g, 56%) as a yellow oil which solidified on standing. ¹H NMR (300 MHz, DMSO-d₆): 1.27 (6H, d), 4.60 (1H, p), 5.25 (2H, s), 6.85 (2H, td), 6.93-7.04 (3H, m), 7.15-7.30 (4H, m), 7.30 (1H, t), 7.37 (1H, d), 10.41 (1H, s). m/z: ES+[M+H]+ 393.

### Tert-butyl (3-(4-(l-benzyl-5-isopropoxy-1H-benzo[d]imidazol-2-yl)-3-chlorophenoxy) propyl)carbamate—Synthetic Example A2

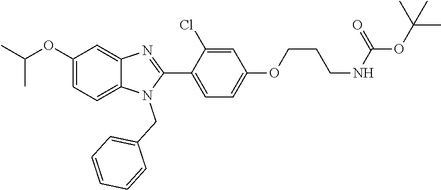 [SMILES: CC(C)Oc1ccc2c(c1)nc(-c1ccc(OCCCNC(=O)OC(C)(C)C)cc1Cl)n2Cc1ccccc1]

DIAD (1.00 g, 4.96 mmol) was added dropwise to 4-(1-benzyl-5-isopropoxy-1H-benzo[d]imidazol-2-yl)-3-chlorophenol (1.5 g, 3.82 mmol), tert-butyl (3-hydroxypropyl)carbamate (0.803 g, 4.58 mmol) and Ph₃P (1.50 g, 5.73 mmol) in THF (20 mL) at 0° C. under nitrogen. The resulting mixture was stirred at rt for 16 hours. The solvent was removed under reduced pressure. The reaction mixture was diluted with EtOAc: petroleum ether (200 mL, 1:5). The solid was filtered out and the organic layer washed sequentially with saturated NH₄Cl (30 mL), saturated NaHCO₃ (30 mL), and saturated brine (30 mL×2). The organic layer was dried over Na₂SO₄, filtered and evaporated to afford a crude product. The crude product was purified by flash silica chromatography, elution gradient 0 to 30% EtOAc in petroleum ether. Pure fractions were evaporated to dryness to afford tert-butyl (3-(4-(1-benzyl-5-isopropoxy-1H-benzo[d]imidazol-2-yl)-3-chlorophenoxy)propyl)carbamate (900 mg, 43%) as a yellow gum. ¹H NMR (400 MHz, CDCl₃): 1.38 (6H, d), 1.46 (9H, s), 2.03 (2H, q), 3.35 (2H, q), 4.07 (2H, t), 4.58 (1H, hept), 4.74 (1H, s), 5.22 (2H, s), 6.87 (2H, ddd), 6.95-7.02 (2H, m), 7.06 (1H, d), 7.09 (1H, d), 7.25 (3H, dd), 7.35 (1H, d), 7.39 (1H, d). m/z: ES+[M+H]+ 550.

### Synthetic Example 4

### 9-Benzyl-8-(2-chloro-4-(2-(4-methylpiperazin-1-yl)ethoxy)phenyl)-6-(1-methylcyclopropoxy)-9H-purine

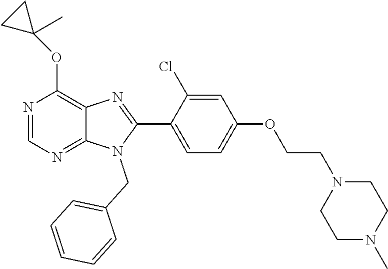 [SMILES: CN1CCN(CCOc2ccc(-c3nc4c(OC5(C)CC5)ncnc4n3Cc3ccccc3)c(Cl)c2)CC1]

To a stirred solution of (E)-N-benzyl-5-((2-chloro-4-(2-(4-methylpiperazin-1-yl)ethoxy)benzylidene)amino)-6-(1-methylcyclopropoxy)pyrimidin-4-amine (82 g crude, calculated as 129 mmol, 1.00 equiv) in IPA (820 mL) was added FeCl₃ (32 g, 193 mmol, 1.50 equiv) under nitrogen atmosphere at room temperature. The reaction mixture was stirred at 80° C. for 1.5 h. The resulting mixture was concentrated under vacuum. The reaction mixture was diluted with 500 ml H₂O, the aqueous layer was adjusted to pH=10 with NaOH and extracted with DCM/IPA (6:1). The combined organic layers were washed with saturated NaHCO₃ and brine (1000 mL×5), dried with Na₂SO₄ and concentrated. The residue was applied onto a silica gel column, eluting with DCM/ammonia solution (3.5M in MeOH) (1:0-1:20). The resulting mixture was further purified by SFC (OptiChiral-C9-5 column, 5 μm silica, 30 mm diameter, 250 mm length) eluting with 50% scCO₂, and MeOH (containing 0.1% 2M NH₃-MeOH) and concentrated under vacuum below 40° C. to afford a yellow solid, which was slurried in Et₂O (10V) for 2 h. The resulting mixture was filtered and the filtrate cake was dried under vacuum to afford 9-benzyl-8-(2-chloro-4-(2-(4-methylpiperazin-1-yl)ethoxy)phenyl)-6-(1-methylcyclopropoxy)-9H-purine (32.4 g, 45%) as a light yellow solid. ¹H NMR (400 MHz, CDCl₃): 0.76-0.88 (2H, m), 1.01-1.26 (2H, m), 1.80 (3H, s), 2.31 (3H, s), 2.37-2.57 (4H, m), 2.64 (4H, br s), 2.85 (2H, t), 4.15 (2H, t), 5.34 (2H, s), 6.82 (1H, dd), 6.93 (2H, dd), 7.06 (1H, d), 7.13-7.21 (2H, m), 7.14-7.19 (2H, m), 8.67 (1H, s). m/z: ES+[M+H]+ 533.

(E)-N-benzyl-5-((2-chloro-4-(2-(4-methylpiperazin-1-yl)ethoxy)benzylidene)amino)-6-(1-methylcyclopropoxy)pyrimidin-4-amine used as starting material was made as follows:

### N-Benzyl-6-chloro-5-nitropyrimidin-4-amine

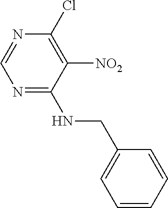 [SMILES: O=[N+]([O-])c1c(Cl)ncnc1NCc1ccccc1]

To a stirred solution of 4,6-dichloro-5-nitropyrimidine (400 g, 2.01 mol, 1.00 equiv) in DCM (4000 mL) was added TEA (228 g, 2.26 mol, 1.1 equiv) under nitrogen atmosphere at room temperature. Phenylmethanamine (243.1 g, 2.26 mol, 1.1 equiv) was charged at 0° C. The reaction mixture was stirred at rt for 30 min. The resulting mixture was washed with brine (1000 mL×5), dried over anhydrous Na₂SO₄ and concentrated. The residue was applied onto a silica gel column with petroleum ether/ethyl acetate (2:1-1:1). This afforded N-benzyl-6-chloro-5-nitropyrimidin-4-amine (327 g, 60%) as a yellow solid. ¹H NMR (300 MHz, CDCl₃): 4.82 (2H, d), 7.28-7.47 (5H, m), 7.82 (1H, s), 8.43 (1H, s). m/z: ES+[M+H]+ 265.

### N-Benzyl-6-(1-methylcyclopropoxy)-5-nitropyrimidin-4-amine

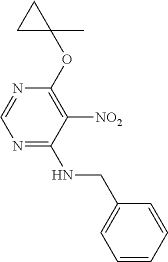 [SMILES: CC1(Oc2ncnc(NCc3ccccc3)c2[N+](=O)[O-])CC1]

To a stirred solution of N-benzyl-6-chloro-5-nitropyrimidin-4-amine (210 g, 0.79 mol, 1.00 equiv) in THF (2100 mL) was added 1-methylcyclopropan-1-ol (114.3 g, 1.59 mmol, 2.00 equiv) under nitrogen atmosphere at room temperature. LHMDS (1980 mL, 1.98 mol, 2.50 equiv) was charged at 0° C. The reaction mixture was stirred at r.t for 18 h. The reaction mixture was diluted with 1000 ml NH₄Cl, extracted with ethyl acetate (1500 mL×3). The combined organic layers were washed with brine (2000 mL×2), dried with Na₂SO₄ and concentrated. The residue was applied onto a silica gel column with petroleum ether/ethyl acetate (1:50-1:20). This afforded N-benzyl-6-(1-methylcyclopropoxy)-5-nitropyrimidin-4-amine (108 g, 45%) as yellow oil. ¹H NMR (400 MHz, DMSO-d₆): 0.76 (2H, t), 0.92 (2H, t), 1.62 (3H, s), 4.70 (2H, d), 7.17-7.26 (1H, m), 7.26-7.39 (4H, m), 8.33 (1H, s), 8.86 (1H, t). m/z: ES+[M+H]+ 301.

### N4-Benzyl-6-(1-methylcyclopropoxy)pyrimidine-4,5-diamine

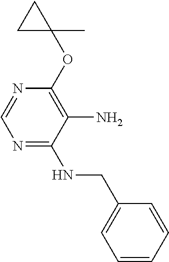 [SMILES: CC1(Oc2ncnc(NCc3ccccc3)c2N)CC1]

To a stirred solution of N-benzyl-6-(1-methylcyclopropoxy)-5-nitropyrimidin-4-amine (108 g, 360 mmol, 1.00 equiv) in EtOH (1080 mL) was added iron powder (201 g, 3.60 mol, 10 equiv) and NH₄Cl (23.2 g, 432 mmol, 1.2 equiv) under nitrogen atmosphere at room temperature. The reaction mixture was stirred at 80° C. for 18 h. The resulting mixture was filtered through celite, eluting with EtOH. The filtrate was concentrated to give crude material. The crude product was purified by flash silica chromatography, eluted with petroleum ether: ethyl acetate 10:1 to 1:3, to afford N4-benzyl-6-(1-methylcyclopropoxy)pyrimidine-4,5-diamine (77 g, 79%) as an off-white solid. ¹H NMR (300 MHz, DMSO-d₆): 0.68 (2H, t), 0.85 (2H, t), 1.60 (3H, s), 4.12 (2H, s), 4.59 (2H, d), 6.69 (1H, t), 7.05-7.41 (5H, m), 7.72 (1H, s). m/z: ES+[M+H]+ 271.

### (E)-N-benzyl-5-((2-chloro-4-(2-(4-methylpiperazin-1-yl)ethoxy)benzylidene)amino)-6-(1-methylcyclopropoxy)pyrimidin-4-amine

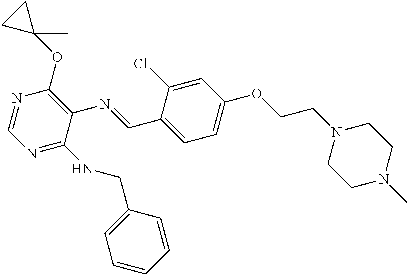 [SMILES: CN1CCN(CCOc2ccc(/C=N/c3c(NCc4ccccc4)ncnc3OC3(C)CC3)c(Cl)c2)CC1]

To a stirred solution of N4-benzyl-6-(1-methylcyclopropoxy)pyrimidine-4,5-diamine (35 g, 129 mmol, 1.00 equiv) in MeOH/AcOH (20V/1V) was added 2-chloro-4-(2-(4-methylpiperazin-1-yl)ethoxy) benzaldehyde (47.6 g, 168 mmol, 1.30 equiv) under nitrogen atmosphere at room temperature. The reaction mixture was stirred at rt for 15 h. The resulting mixture was concentrated under vacuum to afford (E)-N-benzyl-5-((2-chloro-4-(2-(4-methylpiperazin-1-yl)ethoxy)benzylidene)amino)-6-(1-methylcyclopropoxy)pyrimidin-4-amine (82 g, crude) as a yellow oil, which was used directly with no further purification. ¹H NMR (300 MHz, CDCl₃): 0.69-0.81 (2H, m), 0.98-1.10 (2H, m), 1.70 (3H, s), 2.55 (3H, s), 2.73-3.10 (10H, m), 4.08-4.20 (2H, m), 4.75 (2H, d), 6.38-6.49 (1H, m), 6.77-6.88 (1H, m), 6.88-6.97 (1H, m), 7.24-7.38 (5H, m), 8.00 (1H, d), 8.23 (1H, s), 9.40 (1H, s). m/z: ES+[M+H]+ 535.

2-Chloro-4-(2-(4-methylpiperazin-1-yl)ethoxy)benzaldehyde used as starting material was made as follows:

### Tert-butyl 4-(2-(3-chloro-4-formylphenoxy)ethyl)piperazine-1-carboxylate

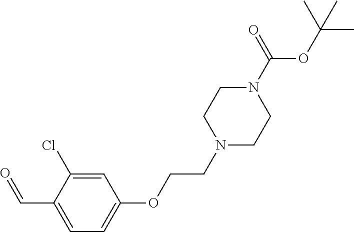 [SMILES: CC(C)(C)OC(=O)N1CCN(CCOc2ccc(C=O)c(Cl)c2)CC1]

To a stirred solution of 2-chloro-4-hydroxybenzaldehyde (300 g, 1.92 mol, 1.00 equiv) in DMF (3000 mL) were added tert-butyl 4-(2-chloroethyl)piperazine-1-carboxylate (643.4 g, 2.59 mol, 1.35 equiv), K₂CO₃ (528.8 g, 3.84 mol, 2.00 equiv) and KI (63.6 g, 0.38 mol, 0.20 equiv) under nitrogen atmosphere. The reaction mixture was stirred at 80° C. for 14 h. The resulting mixture was diluted with 2 L water. The resulting mixture was filtered and the filtrate cake was washed with H₂O. Then the filtrate cake was dried under vacuum to afford tert-butyl 4-(2-(3-chloro-4-formylphenoxy)ethyl)piperazine-1-carboxylate (390 g, 55%) as a yellow solid, which was used in the next step without further purification. ¹H NMR (400 MHz, DMSO-d₆): 1.39 (9H, s), 2.43 (4H, t), 2.73 (2H, t), 3.30 (4H, m), 4.23 (2H, t), 7.08 (1H, dd), 7.20 (1H, d), 7.81 (1H, d), 10.19 (1H, s). m/z: ES+[M+H]+ 369.

### 2-Chloro-4-(2-(4-methylpiperazin-1-yl)ethoxy)benzaldehyde

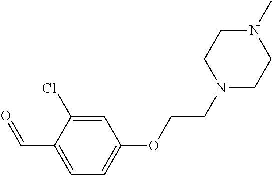 [SMILES: CN1CCN(CCOc2ccc(C=O)c(Cl)c2)CC1]

To a stirred solution of tert-butyl 4-(2-(3-chloro-4-formylphenoxy)ethyl)piperazine-1-carboxylate (390 g, 1.06 mol, 1.00 equiv) in formic acid (1850 mL) was added formaldehyde (48.5 g, 1.28 mol, 1.20 equiv) under nitrogen atmosphere. The reaction mixture was stirred at 70° C. for 14 h. The reaction mixture was diluted with 1000 ml H₂O, extracted with MTBE (1500 mL×3). The aqueous layer was adjusted to pH=10 with NaOH and extracted with DCM (1000 mL×3). The combined organic layers were washed with saturated and brine (1000 mL×5), dried with Na₂SO₄ and concentrated. The residue was purified by flash silica chromatography, eluting with DCM/ammonia solution (3.5M in MeOH) (1:100-1:30). This afforded 2-chloro-4-(2-(4-methylpiperazin-1-yl)ethoxy)benzaldehyde 176 g (42%) as yellow oil. ¹H NMR (300 MHz, DMSO-d₆): 2.14 (3H, s), 2.39 (8H, d), 2.70 (2H, t), 4.22 (2H, t), 7.10 (1H, dd), 7.22 (1H, d), 7.82 (1H, d), 10.20 (1H, d). m/z: ES+[M+H]+ 283.

### Synthetic Example 5

### 9-Benzyl-8-(2-chloro-4-((1-methylpiperidin-4-yl)methoxy)phenyl)-6-(1-methylcyclopropoxy)-9H-purine

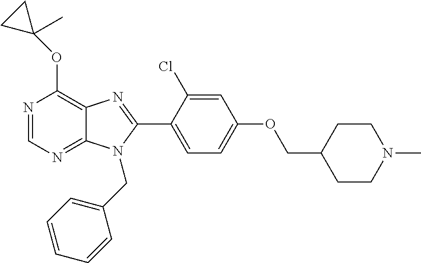 [SMILES: CN1CCC(COc2ccc(-c3nc4c(OC5(C)CC5)ncnc4n3Cc3ccccc3)c(Cl)c2)CC1]

DIAD (143 μL, 0.74 mmol) was added dropwise to 4-(9-benzyl-6-(1-methylcyclopropoxy)-9H-purin-8-yl)-3-chlorophenol (150 mg, 0.37 mmol), (1-methylpiperidin-4-yl)methanol (95 mg, 0.74 mmol) and Ph₃P (193 mg, 0.74 mmol) in THF (5 mL) at 0° C. under nitrogen. The resulting mixture was stirred at rt for 18 hours. The reaction mixture was concentrated and diluted with EtOAc (50 mL) and washed sequentially with saturated NH₄Cl (2×15 mL) and saturated brine (2×15 mL). The organic layer was dried over Na₂SO₄, filtered and evaporated to afford crude product. The residue was purified by preparative TLC (EtOAc), to afford the crude product as a yellow gum. The crude product was purified by preparative HPLC (XBridge Shield RP18 OBD column, 5 μm silica, 30 mm diameter, 150 mm length), using decreasingly polar mixtures of water (containing 0.05% aq. NH₃) and MeCN as eluents. Fractions containing the desired compound were evaporated to dryness to afford 9-benzyl-8-(2-chloro-4-((1-methylpiperidin-4-yl)methoxy)phenyl)-6-(1-methylcyclopropoxy)-9H-purine (5.0 mg, 2.6%) as a white solid. ¹H NMR (400 MHz, DMSO-d₆): 0.80-0.88 (2H, m), 0.98-1.06 (2H, m), 1.21-1.37 (2H, m), 1.64-1.78 (6H, m), 1.80-1.95 (2H, m), 2.17 (3H, s), 2.79 (2H, dd), 3.94 (2H, d), 5.29 (2H, s), 6.86-6.95 (2H, m), 7.00-7.06 (1H, m), 7.16-7.25 (4H, m), 7.40 (1H, d), 8.61 (1H, s). m/z: ES+[M+H]+ 518.

4-(9-Benzyl-6-(1-methylcyclopropoxy)-9H-purin-8-yl)-3-chlorophenol used as starting material was made as follows:

### 9-Benzyl-6-chloro-9H-purine

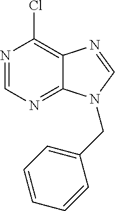 [SMILES: Clc1ncnc2c1ncn2Cc1ccccc1]

(Bromomethyl)benzene (12.2 g, 71.2 mmol) was added dropwise to 6-chloro-9H-purine (10 g, 64.70 mmol), potassium carbonate (10.73 g, 77.64 mmol) in acetonitrile (300 ml-) at 25° C. over a period of 10 minutes. The resulting suspension was stirred at 25° C. for 16 hours. The reaction mixture was filtered through celite and the filtrate was concentrated. The crude product was purified by flash silica chromatography, elution gradient 0 to 50% EtOAc in petroleum ether. Pure fractions were evaporated to dryness to afford 9-benzyl-6-chloro-9H-purine (9.00 g, 57%) as a white solid. ¹H NMR (400 MHz, CDCl3): 5.48 (2H, s), 7.29-7.47 (5H, m), 8.13 (1H, s), 8.81 (1H, s). m/z: ES+[M+H]+ 245.

### 9-Benzyl-6-(1-methylcyclopropoxy)-9H-purine

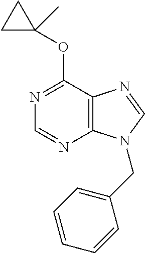 [SMILES: CC1(Oc2ncnc3c2ncn3Cc2ccccc2)CC1]

1-Methylcyclopropan-1-ol (6.52 g, 90.4 mmol) was added to 9-benzyl-6-chloro-9H-purine (8.81 g, 36.0 mmol) in THF (72 ml-) was added. The reaction mixture was stirred at 0° C. and then sodium hydride (60% in mineral oil, 3.62 g, 90.5 mmol) was added slowly to the mixture. The reaction was stirred at room temperature for 17 hours. Water (50 ml) was added slowly and the reaction mixture was extracted with DCM (3×50 mL). The organics were combined and concentrated in vacuo. The crude material was purified by column chromatography on silica gel (330 g, 0-50% EtOAc in heptanes) to yield 9-benzyl-6-(1-methylcyclopropoxy)-9H-purine (7.18 g, 71%) as a yellow gum. ¹H NMR (500 MHz, CDCl₃): 0.8-0.86 (2H, m), 1.1-1.17 (2H, m), 1.78 (3H, s), 5.41 (2H, s), 7.26-7.39 (5H, m), 7.88 (1H, s), 8.64 (1H, s).

m/z: ES+[M+H]+ 281.

### 9-Benzyl-8-bromo-6-(1-methylcyclopropoxy)-9H-purine

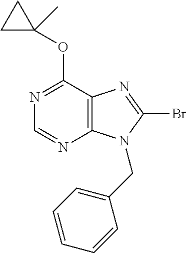 [SMILES: CC1(Oc2ncnc3c2nc(Br)n3Cc2ccccc2)CC1]

Lithium bis(trimethylsilyl)amide (28.6 mL, 28.6 mmol) was added to 9-benzyl-6-(1-methylcyclopropoxy)-9H-purine (5 g, 17.84 mmol) and 1,2-dibromotetrachloroethane (8.72 g, 26.8 mmol) in THF (29 mL) at 0° C. under nitrogen. The resulting mixture was stirred at 25° C. for 16 hours. The reaction mixture was quenched with water (50 mL) and extracted EtOAc (3×100 mL), and saturated brine (1×100 mL). The organic layer was dried over Na₂SO₄, filtered and evaporated to afford crude product. The crude product was purified by flash silica chromatography, elution gradient 0 to 30% EtOAc in petroleum ether. Pure fractions were evaporated to dryness to afford 9-benzyl-8-bromo-6-(1-methylcyclopropoxy)-9H-purine (5.70 g, 89%) as a yellow gum. ¹H NMR (300 MHz, CDCl₃): 0.76-0.87 (2H, m), 1.06-1.16 (2H, m), 1.76 (3H, s), 5.44 (2H, s), 7.27-7.37 (5H, m), 8.60 (1H, s); m/z: ES+[M+H]+ 359.

### 4-(9-Benzyl-6-(1-methylcyclopropoxy)-9H-purin-8-yl)-3-chlorophenol

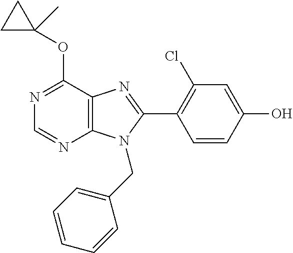 [SMILES: CC1(Oc2ncnc3c2nc(-c2ccc(O)cc2Cl)n3Cc2ccccc2)CC1]

Dichloro[1,1′-bis(diphenylphosphino)ferrocene]palladium(II) (907 mg, 1.39 mmol) was added to 9-benzyl-8-bromo-6-(1-methylcyclopropoxy)-9H-purine (5 g, 13.9 mmol), 3-chloro-4-(4,4,5,5-tetramethyl-1,3,2-dioxaborolan-2-yl)phenol (7.09 g, 27.8 mmol) and cesium carbonate (9.07 g, 27.8 mmol) in 1,4-dioxane (20 mL) and water (2 mL) at 25° C. under nitrogen. The resulting mixture was stirred at 90° C. for 3 hours. The reaction mixture was diluted with EtOAc (50 mL) and washed sequentially with water (3×50 mL). The organic layer was dried over Na₂SO₄, filtered and evaporated to afford a crude product. The crude product was purified by flash silica chromatography, elution gradient 0 to 100% EtOAc in petroleum ether. Pure fractions were evaporated to dryness to afford 4-(9-benzyl-6-(1-methylcyclopropoxy)-9H-purin-8-yl)-3-chlorophenol (5.60 g, 99%) as a yellow solid. ¹H NMR (400 MHz, DMSO-d₆): 0.83 (2H, t), 1.02 (2H, t), 1.73 (3H, s), 5.29 (2H, s), 6.83 (1H, dd), 6.88-6.92 (2H, m), 6.99 (1H, d), 7.18-7.22 (3H, m), 7.29 (1H, d), 8.61 (1H, s)-one H non observed. m/z: ES−[M−H]−405.

### Synthetic Example 6

### 9-Benzyl-8-(2-chloro-4-(2-(piperazin-1-yl)ethoxy)phenyl)-6-(1-methylcyclopropoxy)-9H-purine

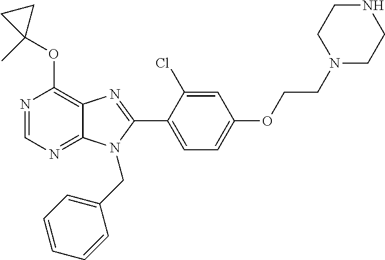 [SMILES: CC1(Oc2ncnc3c2nc(-c2ccc(OCCN4CCNCC4)cc2Cl)n3Cc2ccccc2)CC1]

tert-Butyldimethylsilyltrifluoromethanesulfonate (12.05 mL, 52.5 mmol) was added to tert-butyl 4-(2-(4-(9-benzyl-6-(1-methylcyclopropoxy)-9H-purin-8-yl)-3-chlorophenoxy)ethyl)piperazine-1-carboxylate (6.5 g, 10.50 mmol) in DCM (130 mL). The resulting solution was stirred at rt for 10 minutes. The solvent was removed under reduced pressure. The crude product was purified by flash C18-flash chromatography, elution gradient 30 to 90% MeOH in water (containing 0.1% NH₄HCO₃). Pure fractions were evaporated to dryness to afford 9-benzyl-8-(2-chloro-4-(2-(piperazin-1-yl)ethoxy)phenyl)-6-(1-methylcyclopropoxy)-9H-purine (3.10 g, 57%) as a white foam. ¹H NMR (400 MHz, DMSO-d₆): 0.72-0.9 (2H, m), 0.95-1.12 (2H, m), 1.73 (3H, s), 2.25-2.49 (4H, m), 2.59-2.92 (6H, m), 4.19 (2H, t), 5.30 (2H, s), 6.91 (2H, dd), 7.04 (1H, dd), 7.13-7.29 (4H, m), 7.41 (1H, d), 8.63 (1H, s)-one proton not observed. m/z: ES+[M+H]+ 519.

Tert-butyl 4-(2-(4-(9-benzyl-6-(1-methylcyclopropoxy)-9H-purin-8-yl)-3-chlorophenoxy)ethyl)piperazine-1-carboxylate used as starting material was made as follows:

### tert-Butyl 4-(2-(4-(9-benzyl-6-chloro-9H-purin-8-yl)-3-chlorophenoxy)ethyl)piperazine-1-carboxylate

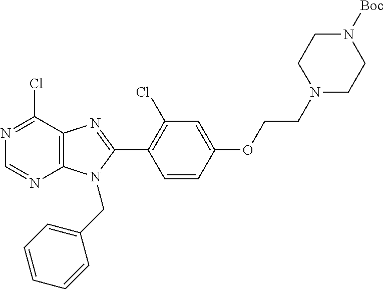 [SMILES: CC(C)(C)OC(=O)N1CCN(CCOc2ccc(-c3nc4c(Cl)ncnc4n3Cc3ccccc3)c(Cl)c2)CC1]

Iron(III) chloride solution (20.73 g, 128 mmol) was added to N4-benzyl-6-chloropyrimidine-4,5-diamine (30 g, 128 mmol, Synthetic Example 1) and tert-butyl 4-(2-(3-chloro-4-formylphenoxy)ethyl)piperazine-1-carboxylate (51.9 g, 141 mmol) in EtOH (500 mL). The resulting mixture was stirred at 60° C. for 2 days. The reaction mixture was evaporated to dryness, redissolved in EtOAc (100 mL) and washed with water (3×100 mL). The organic layer was dried over Na₂SO₄, filtered and evaporated to afford a crude product. The crude product was purified by flash C18 chromatography, elution gradient 40 to 70% MeCN in water. Pure fractions were evaporated to dryness to afford tert-butyl 4-(2-(4-(9-benzyl-6-chloro-9H-purin-8-yl)-3-chlorophenoxy)ethyl)piperazine-1-carboxylate (12.00 g, 16%) as a yellow solid.

¹H NMR (300 MHz, DMSO-d₆): 1.17 (2H, t), 1.40 (9H, s), 1.99 (2H, s), 2.45 (4H, t), 2.74 (2H, t), 4.22 (2H, t), 5.38 (2H, s), 6.89-6.99 (2H, m), 7.09 (1H, dd), 7.17-7.25 (3H, m), 7.29 (1H, d), 7.51 (1H, d), 8.86 (1H, s). m/z: ES+[M+H]+ 583.

### tert-Butyl 4-(2-(4-(9-benzyl-6-(1-methylcyclopropoxy)-9H-purin-8-yl)-3-chlorophenoxy)ethyl)piperazine-1-carboxylate

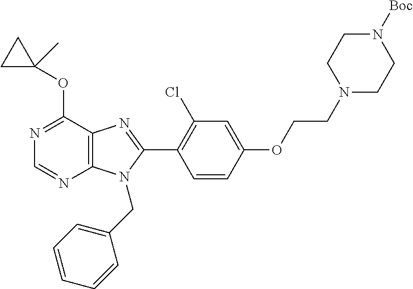 [SMILES: CC(C)(C)OC(=O)N1CCN(CCOc2ccc(-c3nc4c(OC5(C)CC5)ncnc4n3Cc3ccccc3)c(Cl)c2)CC1]

Sodium hydride (6.17 g, 257 mmol) was added to 1-methylcyclopropan-1-ol (5.56 g, 77.12 mmol) and tert-butyl 4-(2-(4-(9-benzyl-6-chloro-9H-purin-8-yl)-3-chlorophenoxy)ethyl)piperazine-1-carboxylate (30 g, 51.4 mmol) in THF (200 mL) at 0° C. under nitrogen. The resulting mixture was stirred at rt for 1 hour. The reaction mixture was poured into ice water. The reaction mixture was evaporated, diluted with EtOAc (250 mL) and washed sequentially with water (3×200 mL) and saturated brine (2×200 mL). The organic layer was dried over Na₂SO₄, filtered and evaporated to afford the crude product. The crude product was purified by flash C18-flash chromatography, elution gradient 30 to 80% MeCN in water (containing 0.1% NH₄HCO₃). Pure fractions were evaporated to dryness to afford tert-butyl 4-(2-(4-(9-benzyl-6-(1-methylcyclopropoxy)-9H-purin-8-yl)-3-chlorophenoxy)ethyl)piperazine-1-carboxylate (13.00 g, 41%) as a yellow solid. ¹H NMR (300 MHz, DMSO-d₆): 0.8-0.88 (2H, m), 0.98-1.06 (2H, m), 1.40 (9H, s), 1.74 (3H, s), 2.39-2.48 (4H, m), 2.69-2.82 (2H, m), 3.33 (4H, s), 4.20 (2H, t), 5.30 (2H, s), 6.85-6.96 (2H, m), 7.05 (1H, d), 7.14-7.29 (4H, m), 7.42 (1H, d), 8.62 (1H, s). m/z: ES+[M+H]+ 619.

Tert-butyl 4-(2-(3-chloro-4-formylphenoxy)ethyl)piperazine-1-carboxylate used as starting material was made as described in Synthetic Example 4.

### Synthetic Example 7

### 4-((8-(2-Chloro-4-(2-(4-methylpiperazin-1-yl)ethoxy)phenyl)-6-(1-methylcyclopropoxy)-9H-purin-9-yl)methyl)-2-methylthiazole

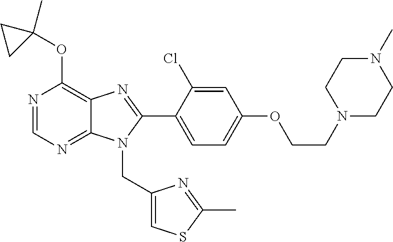 [SMILES: Cc1nc(Cn2c(-c3ccc(OCCN4CCN(C)CC4)cc3Cl)nc3c(OC4(C)CC4)ncnc32)cs1]

A solution of sodium hydrosulfite (282 mg, 1.62 mmol) in water (1 mL) was added to a stirred mixture of 6-(1-methylcyclopropoxy)-N-((2-methylthiazol-4-yl)methyl)-5-nitropyrimidin-4-amine (130 mg, 0.40 mmol) and 2-chloro-4-(2-(4-methylpiperazin-1-yl)ethoxy)benzaldehyde (137 mg, 0.49 mmol, Synthetic Example 4 starting material) in NMP (3 mL). The resulting mixture was stirred at 100° C. for 16 hours then at 110° C. for a further 16 hours. The crude product was purified by flash C18-flash chromatography, elution gradient 5 to 40% MeCN in water (containing 5% TFA). Fractions were evaporated to dryness to afford the crude product. The crude product was further purified by preparative HPLC (XBridge Prep OBD C18 column, 5 μm silica, 30 mm diameter, 150 mm length), using decreasingly polar mixtures of water (containing 0.1% aq. NH₃ and 10 mmol/L NH₄HCO₃) and MeCN as eluents. Fractions containing the desired compound were evaporated to dryness to afford 4-((8-(2-chloro-4-(2-(4-methylpiperazin-1-yl)ethoxy)phenyl)-6-(1-methylcyclopropoxy)-9H-purin-9-yl)methyl)-2-methylthiazole (55 mg, 24%) as a white solid. ¹H NMR (400 MHz, DMSO-d₆): 0.81-0.88 (2H, m), 1.01 (2H, d), 1.73 (3H, s), 2.15 (3H, s), 2.33 (4H, s), 2.49 (3H, s), 2.52 (4H, s), 2.69 (2H, t), 4.17 (2H, t), 5.30 (2H, s), 6.96 (1H, s), 7.02 (1H, dd), 7.22 (1H, d), 7.43 (1H, d), 8.60 (1H, s). m/z: ES+[M+H]+ 554.

6-(1-Methylcyclopropoxy)-N-((2-methylthiazol-4-yl)methyl)-5-nitropyrimidin-4-amine used as starting material was made as follows:

### 6-Chloro-N-((2-methylthiazol-4-yl)methyl)-5-nitropyrimidin-4-amine

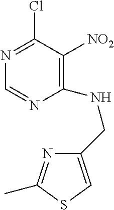 [SMILES: Cc1nc(CNc2ncnc(Cl)c2[N+](=O)[O-])cs1]

A solution of (2-methylthiazol-4-yl)methanamine (330 mg, 2.58 mmol) in DCM (10 mL) was added to a stirred mixture of 4,6-dichloro-5-nitropyrimidine (500 mg, 2.58 mmol) and N,N-diisopropylethylamine (1.35 mL, 7.73 mmol) in DCM (10 mL) at 0° C. The resulting mixture was stirred at rt for 1 hour. The reaction mixture was diluted with water (25 mL), extracted with DCM (3×20 mL). The organic layer was dried over Na₂SO₄, filtered and evaporated to give 6-chloro-N-((2-methylthiazol-4-yl)methyl)-5-nitropyrimidin-4-amine (0.4 g) as a yellow gum. The product was used in the next step directly without further purification.

### 6-(1-methylcyclopropoxy)-N-((2-methylthiazol-4-yl)methyl)-5-nitropyrimidin-4-amine

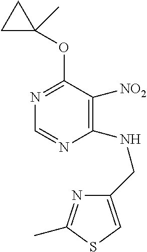 [SMILES: Cc1nc(CNc2ncnc(OC3(C)CC3)c2[N+](=O)[O-])cs1]

LHMDS (2.66 mL, 2.66 mmol) was added to 6-chloro-N-((2-methylthiazol-4-yl)methyl)-5-nitropyrimidin-4-amine (0.38 g, 1.33 mmol) and 1-methylcyclopropan-1-ol (0.192 g, 2.66 mmol) in THF (20 mL) at 0° C. The resulting mixture was stirred at rt for 16 hours. The reaction mixture was quenched with saturated NH₄Cl (25 mL), then the THF solvent was removed under reduced pressure. The reaction mixture was extracted with EtOAc (3×20 mL), the organic layer was dried over Na₂SO₄, filtered and evaporated to afford a yellow gum. The crude product was purified by flash C18-flash chromatography, elution gradient 5 to 100% MeCN in water. Pure fractions were evaporated to dryness to afford 6-(1-methylcyclopropoxy)-N-((2-methylthiazol-4-yl)methyl)-5-nitropyrimidin-4-amine (0.150 g, 35%) as a yellow solid. ¹H NMR (300 MHz, DMSO-d₆): 0.76 (2H, dd), 0.88-0.96 (2H, m), 1.61 (3H, s), 2.61 (3H, s), 4.71 (2H, dd), 7.18 (1H, d), 8.33 (1H, s), 8.80 (1H, t). m/z: ES+[M+H]+ 322.

### Synthetic Example 8

### 1-Benzyl-2-(2-chloro-4-(2-(piperazin-1-yl)ethoxy)phenyl)-5-isopropoxy-1H-imidazo[4,5-b]pyridine

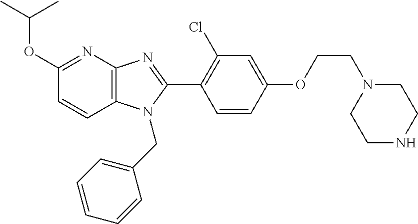 [SMILES: CC(C)Oc1ccc2c(n1)nc(-c1ccc(OCCN3CCNCC3)cc1Cl)n2Cc1ccccc1]

Tert-butyl 4-(2-(4-(1-benzyl-5-isopropoxy-1H-imidazo[4,5-b]pyridin-2-yl)-3-chlorophenoxy)ethyl)piperazine-1-carboxylate (44 mg, 0.07 mmol) was added to trifluoroacetic acid (0.5 ml, 6.49 mmol) in dichloromethane (2 mL). The resulting solution was stirred at rt for 1 hour after which the solvent was removed under reduced pressure. The crude product was purified by flash C18-flash chromatography, elution gradient 30 to 80% MeOH in water (0.1% NH₄HCO₃). Pure fractions were evaporated to dryness to afford 1-benzyl-2-(2-chloro-4-(2-(piperazin-1-yl)ethoxy)phenyl)-5-isopropoxy-1H-imidazo[4,5-b]pyridine (0.026 g, 71%) as a white solid. ¹H NMR (300 MHz, DMSO-d₆): 1.32 (6H, d), 2.40 (4H, t), 2.64-2.72 (6H, m), 4.18 (2H, t), 5.24-5.34 (3H, m), 6.63 (1H, d), 6.94-6.99 (2H, m), 7.07 (1H, dd), 7.21-7.29 (4H, m), 7.49 (1H, d), 7.80 (1H, d)-one proton not observed. m/z: ES+[M+H]+ 506.

Tert-butyl 4-(2-(4-(1-benzyl-5-isopropoxy-1H-imidazo[4,5-b]pyridin-2-yl)-3-chlorophenoxy)ethyl)piperazine-1-carboxylate used as starting material was made as follows:

### 6-Amino-5-nitropyridin-2(1H)-one

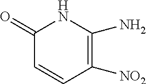 [SMILES: Nc1[nH]c(=O)ccc1[N+](=O)[O-]]

6-Chloro-3-nitropyridin-2-amine (10 g, 57.62 mmol) was added to NaOH (23.1 g, 57.6 mmol) in EtOH (50 mL) and water (16.6 mL). The resulting solution was stirred at 80° C. for 30 minutes. The reaction mixture was acidified with conc. HCl and the precipitate formed was filtered to afford 6-amino-5-nitropyridin-2(1H)-one (8.00 g, 90%) as a yellow solid. The product was used in the next step directly without further purification. ¹H NMR (300 MHz, DMSO-d₆): 11.51 (s, 1H), 8.53 (s, 2H), 7.98 (d, 1H), 5.66 (d, 1H). m/z: ES−[M−H]−154.

### 6-Isopropoxy-3-nitropyridin-2-amine

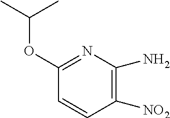

6-Amino-5-nitropyridin-2(1H)-one (4 g, 25.8 mmol) was added to potassium carbonate (10.7 g, 77.4 mmol) and 2-iodopropane (13.1 g, 77.4 mmol) in DMF (80 mL) at 80° C. The resulting solution was stirred at 80° C. overnight. After cooling of the reaction mixture, the solvent was removed under reduced pressure. The crude product was purified by flash silica chromatography, elution gradient 0 to 20% EtOAc in petroleum ether. Pure fractions were evaporated to dryness to afford 6-isopropoxy-3-nitropyridin-2-amine (3.20 g, 63%) as a yellow solid. ¹H NMR (300 MHz, DMSO-d₆): 1.18 (6H, d), 5.12-5.24 (1H, m), 5.95 (1H, d), 7.97 (2H, s), 8.10 (1H, d). m/z: ES+[M+H]+ 198.

### 6-Isopropoxypyridine-2,3-diamine

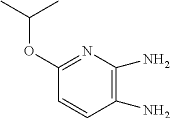

6-Isopropoxy-3-nitropyridin-2-amine (3.1 g, 15.72 mmol) and palladium on carbon (310 mg, 0.29 mmol) in MeOH (60 mL) were stirred under an atmosphere of hydrogen at rt for 2 hours. The reaction mixture was filtered through filter paper to afford 6-isopropoxypyridine-2,3-diamine (2.60 g, 99%) as a purple oil. The product was used in the next step directly without further purification. ¹H NMR (300 MHz, DMSO-d₆): 1.18 (6H, d), 3.92-4.69 (2H, m), 4.9-4.97 (1H, m), 4.97-5.51 (2H, m), 5.73 (1H, d), 6.73 (1H, d). m/z: ES+[M+H]+ 168.

### N3-benzyl-6-isopropoxypyridine-2,3-diamine

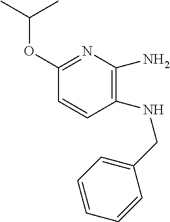 [SMILES: CC(C)Oc1ccc(NCc2ccccc2)c(N)n1]

6-Isopropoxypyridine-2,3-diamine (1.5 g, 8.97 mmol) was added to benzaldehyde (0.909 ml, 8.97 mmol) and acetic acid (0.051 ml, 0.90 mmol) in dichloromethane (30 mL). The reaction was stirred for 5 hours at rt. Sodium triacetoxyborohydride (5.70 g, 26.91 mmol) was then added to the reaction mixture. The resulting solution was stirred for a further 16 hours at rt. The reaction mixture was concentrated under reduced pressure. The crude product was purified by flash silica chromatography, elution gradient 0 to 20% EtOAc in petroleum ether. Pure fractions were evaporated to dryness to afford N3-benzyl-6-isopropoxypyridine-2,3-diamine (1.10 g, 48%) as a purple oil. ¹H NMR (300 MHz, DMSO-d₆): 1.17 (6H, d), 4.20 (2H, s), 4.94 (1H, p), 5.45 (2H, s), 5.73 (1H, d), 6.57 (1H, d), 7.12-7.25 (1H, m), 7.25-7.46 (4H, m)-one proton not observed. m/z: ES+[M+H]+ 258.

### Tert-butyl 4-(2-(4-(1-benzyl-5-isopropoxy-1H-imidazo[4,5-b]pyridin-2-yl)-3-chlorophenoxy)ethyl)piperazine-1-carboxylate

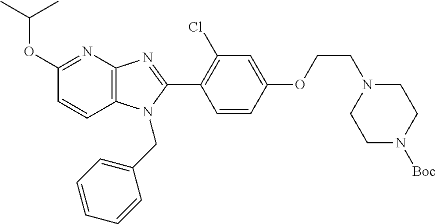 [SMILES: CC(C)Oc1ccc2c(n1)nc(-c1ccc(OCCN3CCN(C(=O)OC(C)(C)C)CC3)cc1Cl)n2Cc1ccccc1]

N3-benzyl-6-isopropoxypyridine-2,3-diamine (50 mg, 0.19 mmol) was added to tert-butyl 4-(2-(3-chloro-4-formylphenoxy)ethyl)piperazine-1-carboxylate (86 mg, 0.23 mmol, Synthetic Example 6 starting material) and copper(II) acetate monohydrate (7.76 mg, 0.04 mmol) in acetic acid (2 mL). The resulting solution was stirred at 100° C. for 1 hour. The solvent was removed under reduced pressure. The crude product was purified by flash silica chromatography, elution gradient 0 to 10% MeOH in dichloromethane. Pure fractions were evaporated to dryness to afford tert-butyl 4-(2-(4-(1-benzyl-5-isopropoxy-1H-imidazo[4,5-b]pyridin-2-yl)-3-chlorophenoxy)ethyl)piperazine-1-carboxylate (118 mg, 100%) as a yellow solid. ¹H NMR (300 MHz, DMSO-d₆): 1.2-1.35 (6H, m), 1.39 (9H, d), 2.44 (4H, s), 2.73 (2H, s), 3.17 (8H, d), 4.08 (3H, q), 4.19 (1H, d), 5.30 (1H, d), 6.82 (1H, t), 6.97 (1H, d), 7.07 (1H, d), 7.2-7.25 (1H, m), 7.27 (1H, d), 7.46-7.56 (1H, m). m/z: ES+[M+H]+ 606.

### Synthetic Example 9

### 2-(2-Chloro-4-(2-(4-methylpiperazin-1-yl)ethoxy)phenyl)-1-(3-chlorobenzyl)-1H-benzo[d]imidazol-5-ol

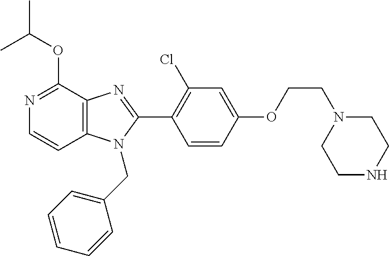 [SMILES: CC(C)Oc1nccc2c1nc(-c1ccc(OCCN3CCNCC3)cc1Cl)n2Cc1ccccc1]

Tert-butyl 4-(2-(4-(1-benzyl-4-isopropoxy-1H-imidazo[4,5-c]pyridin-2-yl)-3-chlorophenoxy)ethyl)piperazine-1-carboxylate (90 mg, 0.15 mmol) was added to trifluoroacetic acid (1 mL, 12.98 mmol) in dichloromethane (4 mL). The resulting solution was stirred at rt for 1 hour. The solvent was removed under reduced pressure. The crude product was purified by flash C18-flash chromatography, elution gradient 30 to 80% MeOH in water (0.1% NH₄HCO₃). Pure fractions were evaporated to dryness to afford 1-benzyl-2-(2-chloro-4-(2-(piperazin-1-yl)ethoxy)phenyl)-4-isopropoxy-1H-imidazo[4,5-c]pyridine (0.029 g, 38%) as a white solid. ¹H NMR (400 MHz, DMSO-d₆): 1.37 (6H, d), 2.36-2.44 (4H, m), 2.64-2.72 (6H, m), 4.18 (2H, t), 5.28 (2H, s), 5.44-5.55 (1H, m), 6.92-6.96 (2H, m), 7.05 (1H, dd), 7.12 (1H, d), 7.21-7.28 (4H, m), 7.47 (1H, d), 7.85 (1H, d)-one proton not observed. m/z: ES+[M+H]+ 506.

Tert-butyl 4-(2-(4-(1-benzyl-4-isopropoxy-1H-imidazo[4,5-c]pyridin-2-yl)-3-chlorophenoxy)ethyl)piperazine-1-carboxylate used as starting material was made as follows:

### 4-Chloro-2-isopropoxy-3-nitropyridine

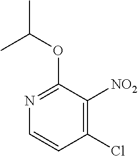

4-Chloro-3-nitropyridin-2(1H)-one (5.0 g, 28.6 mmol) was added to sodium hydride (2.06 g, 85.9 mmol) in DMF (100 mL) at 0° C. The reaction was warmed to rt over 30 minutes and then 2-iodopropane (24.3 g, 143.2 mmol) was added. The resulting solution was stirred at rt for a further 12 hours. The reaction mixture was quenched with saturated NH₄Cl (50 mL), extracted with EtOAc (3×100 mL), the organic layer was dried over Na₂SO₄, filtered and evaporated to afford crude product. The crude product was purified by flash C18-flash chromatography, elution gradient 5 to 80% MeOH in water (0.1% NH₄HCO₃). Pure fractions were evaporated to dryness to afford 4-chloro-2-isopropoxy-3-nitropyridine (2.10 g, 34%) as a yellow oil. ¹H NMR (300 MHz, DMSO-d₆): 1.30 (6H, d), 5.29-5.44 (1H, m), 7.43 (1H, d), 8.37 (1H, d).

### N-Benzyl-2-isopropoxy-3-nitropyridin-4-amine

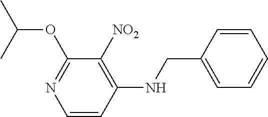 [SMILES: CC(C)Oc1nccc(NCc2ccccc2)c1[N+](=O)[O-]]

4-Chloro-2-isopropoxy-3-nitropyridine (1.0 g, 4.62 mmol) was added to benzylamine (0.504 mL, 4.62 mmol) and TEA (6.43 mL, 46.2 mmol) in DMSO (20 mL). The resulting solution was stirred at 90° C. for 5 hours. The solvent was removed under reduced pressure. The crude product was purified by flash silica chromatography, elution gradient 0 to 10% EtOAc in petroleum ether. Pure fractions were evaporated to dryness to afford N-benzyl-2-isopropoxy-3-nitropyridin-4-amine (1.20 g, 90%) as a yellow solid. ¹H NMR (300 MHz, DMSO-d₆): 1.25 (6H, d), 4.49 (2H, d), 5.21-5.35 (1H, m), 6.39 (1H, d), 7.21-7.28 (1H, m), 7.3-7.35 (4H, m), 7.75 (1H, d), 7.87 (1H, t). m/z: ES−[M−H]−286.

### N4-Benzyl-2-isopropoxypyridine-3,4-diamine

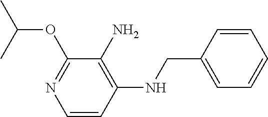 [SMILES: CC(C)Oc1nccc(NCc2ccccc2)c1N]

N-benzyl-2-isopropoxy-3-nitropyridin-4-amine (1 g, 3.48 mmol) was added to iron (0.972 g, 17.40 mmol) and ammonium chloride (1.862 g, 34.80 mmol) in EtOH (16 mL) and water (1.6 mL). The resulting solution was stirred at 80° C. for 12 hours. The reaction mixture was filtered through filter paper then the solvents were removed under reduced pressure. The crude product was purified by flash silica chromatography, elution gradient 0 to 20% EtOAc in petroleum ether. Pure fractions were evaporated to dryness to afford N4-benzyl-2-isopropoxypyridine-3,4-diamine (0.518 g, 58%) as a yellow solid. ¹H NMR (300 MHz, DMSO-d₆): 1.25 (6H, d), 4.04 (2H, s), 4.35 (2H, d), 5.09-5.19 (1H, m), 5.91 (1H, t), 6.10 (1H, d), 7.18 (1H, d), 7.2-7.25 (1H, m), 7.28-7.32 (1H, m), 7.32-7.37 (3H, m). m/z: ES+[M+H]+ 258.

### Tert-butyl 4-(2-(4-(1-benzyl-4-isopropoxy-1H-imidazo[4,5-c]pyridin-2-yl)-3-chlorophenoxy)ethyl)piperazine-1-carboxylate

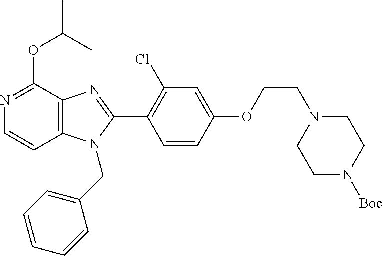 [SMILES: CC(C)Oc1nccc2c1nc(-c1ccc(OCCN3CCN(C(=O)OC(C)(C)C)CC3)cc1Cl)n2Cc1ccccc1]

N4-benzyl-2-isopropoxypyridine-3,4-diamine (50 mg, 0.19 mmol) was added to tert-butyl 4-(2-(3-chloro-4-formylphenoxy)ethyl)piperazine-1-carboxylate (86 mg, 0.23 mmol) and copper(II) acetate monohydrate (7.8 mg, 0.04 mmol) in AcOH (2 mL). The resulting solution was stirred at 100° C. for 1 hour. The solvent was removed under reduced pressure. The crude product was purified by flash C18-flash chromatography, elution gradient 10 to 80% MeOH in water (0.1% NH₄HCO₃). Pure fractions were evaporated to dryness to afford tert-butyl 4-(2-(4-(1-benzyl-4-isopropoxy-1H-imidazo[4,5-c]pyridin-2-yl)-3-chlorophenoxy)ethyl)piperazine-1-carboxylate (95 mg, 81%) as a yellow solid. ¹H NMR (400 MHz, DMSO-d₆): 1.37 (6H, d), 1.39 (9H, s), 2.44 (4H, t), 2.74 (2H, t), 3.28-3.3 (2H, m), 3.32-3.34 (2H, m), 4.20 (2H, t), 5.28 (2H, s), 5.47-5.53 (1H, m), 6.92-6.96 (2H, m), 7.05 (1H, dd), 7.12 (1H, d), 7.21-7.24 (3H, m), 7.27 (1H, d), 7.47 (1H, d), 7.85 (1H, d). m/z: ES+[M+H]+ 606.

### Synthetic Example 10

### 8-(2-Chloro-4-(2-(piperazin-1-yl)ethoxy)phenyl)-9-((4-chloropyridin-2-yl)methyl)-6-(1-methylcyclopropoxy)-9H-purine

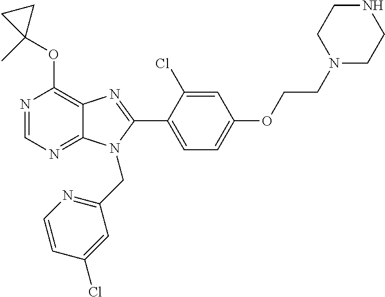 [SMILES: CC1(Oc2ncnc3c2nc(-c2ccc(OCCN4CCNCC4)cc2Cl)n3Cc2cc(Cl)ccn2)CC1]

A solution of tert-butyl 4-(2-(3-chloro-4-(9-((4-chloropyridin-2-yl)methyl)-6-(1-methylcyclopropoxy)-9H-purin-8-yl)phenoxy)ethyl)piperazine-1-carboxylate (2.07 g, 3.16 mmol) in acetonitrile was cooled to 0° C. with an ice/water bath. Hydrogen chloride 4.0 M in dioxane (8.0 mL, 32.0 mmol) was added and the ice bath removed. Stirring was continued at rt for 1 h before additional hydrogen chloride 4.0 M in dioxane (8.0 mL, 32.0 mmol) was added and stirring continued for a further 30 min. The reaction mixture was evaporated to give the crude product as a yellow solid. The crude product was purified by ion exchange chromatography, using an SCX column. The desired product was eluted from the column using 1M NH₃/MeOH to dryness to afford the crude free base. The crude free base was purified by flash deactivated alumina chromatography, elution gradient 0 to 10% MeOH in DCM to afford 8-(2-chloro-4-(2-(piperazin-1-yl)ethoxy)phenyl)-9-((4-chloropyridin-2-yl)methyl)-6-(1-methylcyclopropoxy)-9H-purine (1.50 g, 86%) as a yellow foam.

¹H NMR (500 MHz, CDCl₃): 0.8-0.87 (2H, m), 1.14-1.2 (2H, m), 1.81 (3H, s), 2.55 (4H, s), 2.80 (2H, t), 2.92 (4H, t), 4.12 (2H, t), 5.44 (2H, s), 6.80 (1H, dd), 6.88 (1H, dd), 7.02 (1H, d), 7.12 (1H, dd), 7.25 (1H, d), 8.30 (1H, dd), 8.64 (1H, s). 1H not observed. m/z: ES+[M+H]+ 554.

Tert-butyl 4-(2-(3-chloro-4-(9-((4-chloropyridin-2-yl)methyl)-6-(1-methylcyclopropoxy)-9H-purin-8-yl)phenoxy)ethyl)piperazine-1-carboxylate used as starting material was made as follows:

### Tert-butyl 4-(2-(3-chloro-4-(6-chloro-9H-purin-8-yl)phenoxy)ethyl)piperazine-1-carboxylate

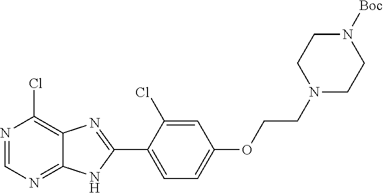 [SMILES: CC(C)(C)OC(=O)N1CCN(CCOc2ccc(-c3nc4c(Cl)ncnc4[nH]3)c(Cl)c2)CC1]

Tert-butyl 4-(2-(3-chloro-4-formylphenoxy)ethyl)piperazine-1-carboxylate (1 g, 2.71 mmol) and 6-chloropyrimidine-4,5-diamine (0.431 g, 2.98 mmol) were dissolved in IPA (38.7 mL). Iron(III) chloride (0.088 g, 0.54 mmol) was added and the reaction stirred at 80° C. under air for 2.5 days. The reaction was cooled to rt, diluted with DCM (50 mL) and water (50 mL) was added. The mixture was filtered through a small plug of celite to aid separation, washing the celite with DCM (50 mL). The mixture was then extracted with DCM (50 mL×3) and the combined organics were washed with saturated NaHCO₃ (20 mL), separated, dried over MgSO₄, filtered and evaporated to afford crude product. The crude product was purified by flash silica chromatography, elution gradient 0 to 100% EtOAc in heptane then 3:1 EtOAc/EtOH in EtOAc to elute product. Pure fractions were evaporated to dryness to afford tert-butyl 4-(2-(3-chloro-4-(6-chloro-9H-purin-8-yl)phenoxy)ethyl)piperazine-1-carboxylate (1.050 g, 78%) as a pale yellow solid. ¹H NMR (500 MHz, DMSO-d₆): 1.40 (9H, s), 2.44-2.48 (4H, m), 2.76 (2H, t), 3.26-3.38 (4H, m), 4.23 (2H, t), 7.15 (1H, dd), 7.30 (1H, d), 7.81 (1H, d), 8.75 (1H, s). 1H not observed. m/z: ES+[M+H]+ 493.

Tert-butyl 4-(2-(3-chloro-4-formylphenoxy)ethyl)piperazine-1-carboxylate used as starting material was made as described in Synthetic Example 4.

### Tert-butyl 4-(2-(3-chloro-4-(6-chloro-9-((4-chloropyridin-2-yl)methyl)-9H-purin-8-yl)phenoxy)ethyl)piperazine-1-carboxylate

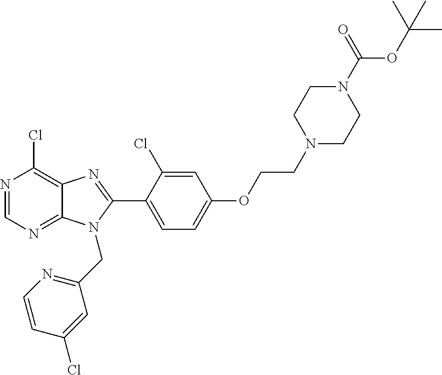 [SMILES: CC(C)(C)OC(=O)N1CCN(CCOc2ccc(-c3nc4c(Cl)ncnc4n3Cc3cc(Cl)ccn3)c(Cl)c2)CC1]

To a solution of tert-butyl 4-(2-(3-chloro-4-(6-chloro-9H-purin-8-yl)phenoxy)ethyl)piperazine-1-carboxylate (1.62 g, 3.28 mmol) and 4-chloro-2-(chloromethyl)pyridine hydrochloride (0.977 g, 4.93 mmol) in anhydrous DMF (16.4 mL) was added cesium carbonate (3.21 g, 9.85 mmol). The reaction was inerted by the application of three cycles of vacuum and nitrogen backfill and then the reaction was stirred at 60° C. under nitrogen for 19 h. The reaction was quenched with ice-water (20 mL), and ethyl acetate (20 mL) was added. The organic layer was removed and the aqueous layer was further extracted with ethyl acetate (10 mL×6). The combined organic layers were washed with a LiCl saturated aqueous solution (10 mL×3), filtered through an isolute phase separation cartridge and evaporated to afford crude product as a brown foam. The crude product was purified by flash silica chromatography, elution gradient 0 to 35% then to 70% 3:1 EtOAc:EtOH in n-heptane. Pure fractions were evaporated to dryness to afford tert-butyl 4-(2-(3-chloro-4-(6-chloro-9-((4-chloropyridin-2-yl)methyl)-9H-purin-8-yl)phenoxy)ethyl)piperazine-1-carboxylate (1.13 g, 56%) as a pale brown foam. ¹H NMR (500 MHz, CDCl₃): 1.47 (9H, s), 2.5-2.54 (4H, m), 2.83 (2H, t), 3.44-3.48 (4H, m), 4.14 (2H, t), 5.47 (2H, s), 6.84 (1H, dd), 6.98 (1H, d), 7.05 (1H, d), 7.14 (1H, dd), 7.31 (1H, d), 8.28 (1H, d), 8.77 (1H, s). m/z: ES+[M+H]+ 618.

### Tert-butyl 4-(2-(3-chloro-4-(9-((4-chloropyridin-2-yl)methyl)-6-(1-methylcyclopropoxy)-9H-purin-8-yl)phenoxy)ethyl)piperazine-1-carboxylate

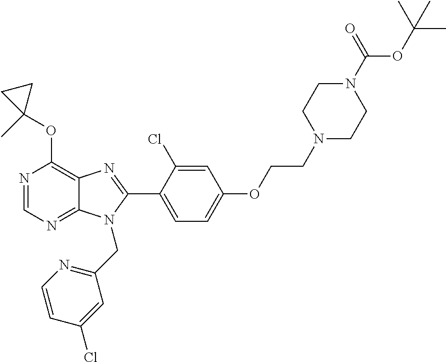 [SMILES: CC(C)(C)OC(=O)N1CCN(CCOc2ccc(-c3nc4c(OC5(C)CC5)ncnc4n3Cc3cc(Cl)ccn3)c(Cl)c2)CC1]

To a suspension of sodium hydride (0.889 g, 22.2 mmol) in tetrahydrofuran (15 mL) cooled to 0° C. under nitrogen was added a solution of 1-methylcyclopropan-1-ol (0.802 g, 11.1 mmol) and tert-butyl 4-(2-(3-chloro-4-(6-chloro-9-((4-chloropyridin-2-yl)methyl)-9H-purin-8-yl)phenoxy)ethyl)piperazine-1-carboxylate (3.44 g, 5.56 mmol) in tetrahydrofuran (30 mL) as a steady stream. The reaction mixture was stirred at 0° C. for 20 min and then the ice bath removed and the reaction was stirred at room temperature under nitrogen for 19 h. The reaction mixture was cooled with an ice/water bath and carefully quenched with saturated ammonium chloride solution (15 mL). After gas evolution had subsided, the mixture was diluted with water (150 mL) and EtOAc (150 mL). The aqueous phase was extracted with EtOAc (150 mL). The combined organic phases were washed with brine, dried and evaporated. The crude product was purified by flash silica chromatography, elution gradient 0 to 100% 3:1 EtOAc/EtOH in heptane to afford tert-butyl 4-(2-(3-chloro-4-(9-((4-chloropyridin-2-yl)methyl)-6-(1-methylcyclopropoxy)-9H-purin-8-yl)phenoxy)ethyl)piperazine-1-carboxylate (2.07 g, 57%) as a pale yellow foam. ¹H NMR (500 MHz, CDCl₃): 0.8-0.87 (2H, m), 1.14-1.2 (2H, m), 1.47 (9H, s), 1.81 (3H, s), 2.45-2.59 (4H, m), 2.82 (2H, t), 3.38-3.53 (4H, m), 4.12 (2H, t), 5.44 (2H, s), 6.80 (1H, dd), 6.89 (1H, d), 7.01 (1H, d), 7.12 (1H, dd), 7.24-7.28 (1H, m), 8.30 (1H, d), 8.64 (1H, s). m/z: ES+[M+H]+ 654.

### Synthetic Example 11

### 9-Benzyl-8-(2-chloro-4-(2-(piperazin-1-yl)ethoxy)phenyl)-6-((1,1,1-trifluoro-2-methylpropan-2-yl)oxy)-9H-purine

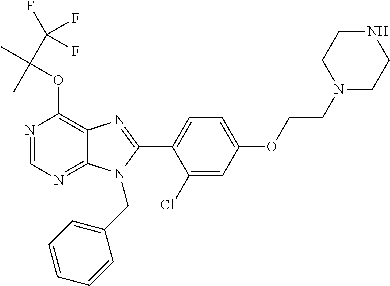 [SMILES: CC(C)(Oc1ncnc2c1nc(-c1ccc(OCCN3CCNCC3)cc1Cl)n2Cc1ccccc1)C(F)(F)F]

Tert-butyl 4-(2-(4-(9-benzyl-6-((1,1,1-trifluoro-2-methylpropan-2-yl)oxy)-9H-purin-8-yl)-3-chlorophenoxy)ethyl)piperazine-1-carboxylate (120 mg, 0.18 mmol) was added to trifluoroacetic acid (0.2 mL, 2.60 mmol) in dichloromethane (2 mL). The resulting solution was stirred at rt for 2 hours. The solvent was removed under reduced pressure. The crude product was purified by flash C18-flash chromatography, elution gradient 50 to 100% MeOH in water (0.1% NH₄HCO₃), followed by preparative HPLC (XBridge Prep OBD C18 column, 30*150 mm, 5 μm), using decreasingly polar mixtures of water (containing 10 mmol/L NH₄HCO₃) and MeCN as eluents. Fractions containing the desired compound were evaporated to dryness to afford 9-benzyl-8-(2-chloro-4-(2-(piperazin-1-yl)ethoxy)phenyl)-6-((1,1,1-trifluoro-2-methylpropan-2-yl)oxy)-9H-purine (30.0 mg, 29%) as a white solid. ¹H NMR (300 MHz, DMSO-d₆): 1.95 (6H, s), 2.3-2.45 (4H, m), 2.58-2.79 (6H, m), 4.18 (2H, t), 5.30 (2H, s), 6.91 (2H, dd), 7.04 (1H, dd), 7.15-7.23 (3H, m), 7.27 (1H, d), 7.43 (1H, d), 8.61 (1H, s)-1H not observed. m/z: ES+[M+H]+ 575.

Tert-butyl 4-(2-(4-(9-benzyl-6-((1,1,1-trifluoro-2-methylpropan-2-yl)oxy)-9H-purin-8-yl)-3-chlorophenoxy)ethyl)piperazine-1-carboxylate used as starting material was made as follows:

### N-benzyl-5-nitro-6-((1,1,1-trifluoro-2-methylpropan-2-yl)oxy)pyrimidin-4-amine

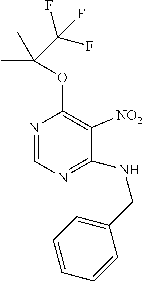 [SMILES: CC(C)(Oc1ncnc(NCc2ccccc2)c1[N+](=O)[O-])C(F)(F)F]

LHMDS (11.3 mL, 11.3 mmol) was added to N-benzyl-6-chloro-5-nitropyrimidin-4-amine (1 g, 3.78 mmol) and 1,1,1-trifluoro-2-methylpropan-2-ol (1.24 mL, 11.3 mmol) in THF (20 mL) under 0° C. The resulting mixture was stirred at 60° C. for 12 hours. The reaction mixture was diluted with EtOAc (100 mL) and washed sequentially with saturated brine (3×100 mL). The organic layer was dried over Na₂SO₄, filtered and evaporated to afford crude product. The crude product was purified by flash silica chromatography, elution gradient 0 to 5% EtOAc in petroleum ether. Fractions were evaporated to dryness to afford N-benzyl-5-nitro-6-((1,1,1-trifluoro-2-methylpropan-2-yl)oxy)pyrimidin-4-amine (800 mg, 59%) as a pale yellow oil. ¹H NMR (300 MHz, DMSO-d₆): 1.81 (6H, s), 4.69 (2H, d), 7.07-7.47 (5H, m), 8.31 (1H, s), 8.90 (1H, t). m/z: ES+[M+H]+ 357.

### N4-benzyl-6-((1,1,1-trifluoro-2-methylpropan-2-yl)oxy)pyrimidine-4,5-diamine

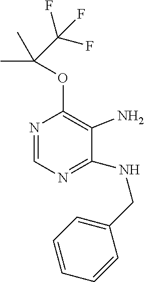 [SMILES: CC(C)(Oc1ncnc(NCc2ccccc2)c1N)C(F)(F)F]

Iron (549 mg, 9.82 mmol) was added to a mixture of N-benzyl-5-nitro-6-((1,1,1-trifluoro-2-methylpropan-2-yl)oxy)pyrimidin-4-amine (700 mg, 1.96 mmol) and ammonium chloride (1.05 g, 19.6 mmol) in ethanol (15 mL). The resulting mixture was stirred at 80° C. for 4 hours. The solvent was then removed under reduced pressure and the crude product was purified by flash silica chromatography, elution gradient 0 to 30% EtOAc in petroleum ether. Pure fractions were evaporated to dryness to afford N4-benzyl-6-((1,1,1-trifluoro-2-methylpropan-2-yl)oxy)pyrimidine-4,5-diamine (400 mg, 63%) as a white solid. ¹H NMR (300 MHz, DMSO-d₆): 1.72 (6H, s), 4.16 (2H, s), 4.61 (2H, d), 6.95 (1H, t), 7.11-7.46 (5H, m), 7.72 (1H, s). m/z: ES+[M+H]+ 327.

### Tert-butyl 4-(2-(4-(9-benzyl-6-((1,1,1-trifluoro-2-methylpropan-2-yl)oxy)-9H-purin-8-yl)-3-chlorophenoxy)ethyl)piperazine-1-carboxylate

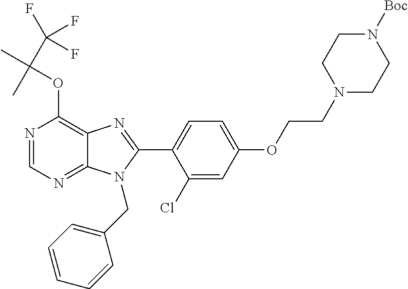 [SMILES: CC(C)(C)OC(=O)N1CCN(CCOc2ccc(-c3nc4c(OC(C)(C)C(F)(F)F)ncnc4n3Cc3ccccc3)c(Cl)c2)CC1]

Iron(III) chloride (49.7 mg, 0.31 mmol) was added to N4-benzyl-6-((1,1,1-trifluoro-2-methylpropan-2-yl)oxy)pyrimidine-4,5-diamine (100 mg, 0.31 mmol), tert-butyl 4-(2-(3-chloro-4-formylphenoxy)ethyl)piperazine-1-carboxylate (124 mg, 0.34 mmol) and AcOH (1.7 μL, 0.03 mmol) in IPA (2 mL). The resulting mixture was stirred at 80° C. for 4 hours. The solvent was then removed under reduced pressure and the crude residue was purified by C18-flash chromatography, elution gradient 40 to 90% MeOH in water (0.1% NH₄HCO₃). Pure fractions were evaporated to dryness to afford tert-butyl 4-(2-(4-(9-benzyl-6-((1,1,1-trifluoro-2-methylpropan-2-yl)oxy)-9H-purin-8-yl)-3-chlorophenoxy)ethyl)piperazine-1-carboxylate (140 mg, 68%) as a white foam. ¹H NMR (300 MHz, DMSO-d₆): 1.38 (9H, s), 1.94 (6H, s), 2.43 (4H, t), 2.72 (2H, t), 3.24-3.32 (4H, m), 4.19 (2H, t), 5.29 (2H, s), 6.90 (2H, dd), 7.03 (1H, dd), 7.12-7.24 (3H, m), 7.26 (1H, d), 7.43 (1H, d), 8.60 (1H, s). m/z: ES+[M+H]+ 675.

### Synthetic Example 12

### 9-Benzyl-8-(2-chloro-4-(2-(piperazin-1-yl)ethoxy)phenyl)-6-(1-methylcyclobutoxy)-9H-purine

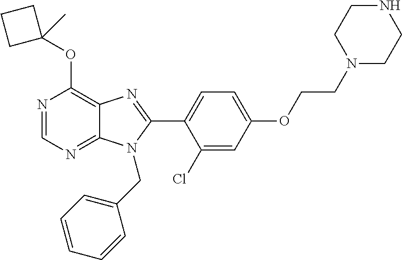 [SMILES: CC1(Oc2ncnc3c2nc(-c2ccc(OCCN4CCNCC4)cc2Cl)n3Cc2ccccc2)CCC1]

Pd(PPh₃)₄ (31.0 mg, 0.03 mmol) was added to 9-benzyl-8-bromo-6-(1-methylcyclobutoxy)-9H-purine (100 mg, 0.27 mmol), 1-(2-(3-chloro-4-(4,4,5,5-tetramethyl-1,3,2-dioxaborolan-2-yl)phenoxy)ethyl)piperazine (196 mg, 0.54 mmol) and Cs₂CO₃ (262 mg, 0.80 mmol) in 1,4-dioxane (2 mL) and water (0.4 mL) under nitrogen. The resulting solution was stirred at 100° C. for 1 hour. The solvent was removed under reduced pressure. The crude product was purified by flash C18-flash chromatography, elution gradient 50 to 100% MeOH in water (0.1% NH₄HCO₃), followed by preparative HPLC (Phenomenex Gemini-NX axia Prep C18 OBD column, 5 μm silica, 19 mm diameter, 100 mm length), using decreasingly polar mixtures of water (containing 0.1% NH₃) and MeCN as eluents. Fractions containing the desired compound were evaporated to dryness to afford 9-benzyl-8-(2-chloro-4-(2-(piperazin-1-yl)ethoxy)phenyl)-6-(1-methylcyclobutoxy)-9H-purine (16 mg, 11%) as a white solid. ¹H NMR (300 MHz, DMSO-d₆): 1.62-1.96 (5H, m), 2.26-2.49 (8H, m), 2.61-2.78 (6H, m), 4.18 (2H, t), 5.28 (2H, s), 6.85-6.95 (2H, m), 6.98-7.11 (1H, m), 7.15-7.25 (3H, m), 7.26 (1H, d), 7.41 (1H, d), 8.51 (1H, s)-one H not observed. m/z: ES+[M+H]+ 533.

1-(2-(3-Chloro-4-(4,4,5,5-tetramethyl-1,3,2-dioxaborolan-2-yl)phenoxy)ethyl)piperazine used as starting material was made as follows:

### Tert-butyl 4-(2-(4-bromo-3-chlorophenoxy)ethyl)piperazine-1-carboxylate

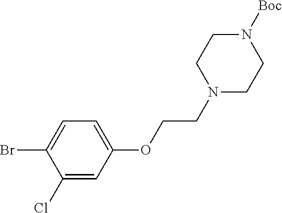 [SMILES: CC(C)(C)OC(=O)N1CCN(CCOc2ccc(Br)c(Cl)c2)CC1]

Potassium carbonate (6.66 g, 48.2 mmol) was added to 4-bromo-3-chlorophenol (5 g, 24.1 mmol), tert-butyl 4-(2-chloroethyl)piperazine-1-carboxylate (7.19 g, 28.92 mmol) in DMF (100 mL) at 25° C. under nitrogen. The resulting suspension was stirred at 80° C. for 3 hours. The reaction mixture was diluted with water (300 mL), and the aqueous layer was extracted with EtOAc (3×100 mL). The organic layer was dried over Na₂SO₄, filtered and evaporated to afford the crude product. The crude product was purified by crystallisation from EtOAc/petroleum ether to afford tert-butyl 4-(2-(4-bromo-3-chlorophenoxy)ethyl)piperazine-1-carboxylate (5.50 g, 54%) as a white solid. ¹H NMR (300 MHz, CDCl₃): 1.46 (9H, s), 2.51 (4H, s), 2.81 (2H, t), 3.45 (4H, t), 4.07 (2H, t), 6.70 (1H, dd), 7.02 (1H, d), 7.47 (1H, d). m/z: ES+[M+H]+ 419.

### Tert-butyl 4-(2-(3-chloro-4-(4,4,5,5-tetramethyl-1,3,2-dioxaborolan-2-yl)phenoxy)ethyl)piperazine-1-carboxylate

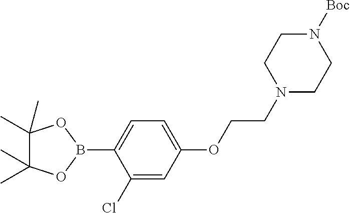 [SMILES: CC(C)(C)OC(=O)N1CCN(CCOc2ccc(B3OC(C)(C)C(C)(C)O3)c(Cl)c2)CC1]

PdCl₂(dppf) (0.523 g, 0.71 mmol) was added to tert-butyl 4-(2-(4-bromo-3-chlorophenoxy)ethyl)piperazine-1-carboxylate (3 g, 7.15 mmol), bis(pinacolato)diboron (3.63 g, 14.29 mmol) and potassium acetate (2.10 g, 21.4 mmol) in 1,4-dioxane (60 mL) under nitrogen. The resulting solution was stirred at 100° C. for 2 hours. The reaction mixture was diluted with EtOAc (250 mL), and washed sequentially with water (3×250 mL) and saturated brine (3×250 mL). The organic layer was dried over Na₂SO₄, filtered and evaporated to afford the crude product. The crude product was purified by flash silica chromatography, elution gradient 0 to 30% EtOAc in petroleum ether. Pure fractions were evaporated to dryness to afford tert-butyl 4-(2-(3-chloro-4-(4,4,5,5-tetramethyl-1,3,2-dioxaborolan-2-yl)phenoxy)ethyl)piperazine-1-carboxylate (3.00 g, 90%) as a pale yellow gum. ¹H NMR (300 MHz, DMSO-d₆): 1.16 (9H, s), 1.27 (12H, s), 2.42 (4H, m), 2.58 (2H, t), 3.17-3.20 (4H, m), 4.01 (2H, t), 6.79 (1H, dd), 6.88 (1H, d), 7.46 (1H, d). m/z: ES+[M+H]+ 467.

### 1-(2-(3-Chloro-4-(4,4,5,5-tetramethyl-1,3,2-dioxaborolan-2-yl)phenoxy)ethyl)piperazine

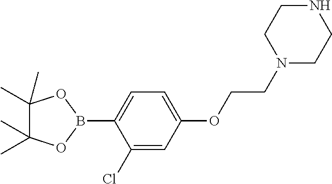 [SMILES: CC1(C)OB(c2ccc(OCCN3CCNCC3)cc2Cl)OC1(C)C]

Trifluoroacetic acid (20 mL) was added to tert-butyl 4-(2-(3-chloro-4-(4,4,5,5-tetramethyl-1,3,2-dioxaborolan-2-yl)phenoxy)ethyl)piperazine-1-carboxylate (2 g, 4.28 mmol) in dichloromethane (20 mL). The resulting solution was stirred at 25° C. for 2 hours and then the solvent was removed under reduced pressure. The crude product was purified by preparative HPLC (XBridge Prep C18 OBD column, 30*150 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness to afford 1-(2-(3-chloro-4-(4,4,5,5-tetramethyl-1,3,2-dioxaborolan-2-yl)phenoxy)ethyl)piperazine (1.05 g, 67%) as a white solid. ¹H NMR (300 MHz, DMSO-d₆): 1.29 (12H, s), 3.20 (4H, t), 3.30 (6H, t), 4.30 (2H, t), 6.95 (1H, dd), 7.05 (1H, d), 7.62 (1H, d)-one proton not observed. m/z: ES+[M+H]+ 367.

9-Benzyl-8-bromo-6-(1-methylcyclobutoxy)-9H-purine used as starting material was made as follows:

### 9-Benzyl-6-(1-methylcyclobutoxy)-9H-purine

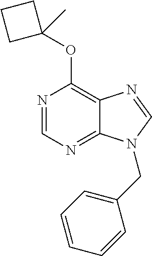 [SMILES: CC1(Oc2ncnc3c2ncn3Cc2ccccc2)CCC1]

Sodium hydride (265 mg, 11.03 mmol) was added portionwise to a mixture of 9-benzyl-6-chloro-9H-purine (900 mg, 3.68 mmol, Synthetic Example 5 starting material), 1-methylcyclobutan-1-ol (634 mg, 7.36 mmol) in THF (30 mL) at 0° C. over a period of 2 minutes under nitrogen. The resulting suspension was stirred at 25° C. for 4 hours. The reaction mixture was diluted with aqueous NH₄Cl (3 mL) and water (50 mL). The aqueous layer was extracted with EtOAc (3×25 mL). The combined organic layers were dried over Na₂SO₄, filtered and evaporated to afford the crude product. The crude product was purified by flash silica chromatography, elution gradient 0 to 40% EtOAc in petroleum ether. Pure fractions were evaporated to dryness to afford 9-benzyl-6-(1-methylcyclobutoxy)-9H-purine (900 mg, 83%) as a colourless solid. ¹H NMR (300 MHz, CDCl₃): 1.64-1.94 (5H, m), 2.29-2.42 (2H, m), 2.52-2.67 (2H, m), 5.41 (2H, s), 7.24-7.38 (5H, m), 7.90 (1H, s), 8.51 (1H, s). m/z: ES+[M+H]+ 295.

### 9-Benzyl-8-bromo-6-(1-methylcyclobutoxy)-9H-purine

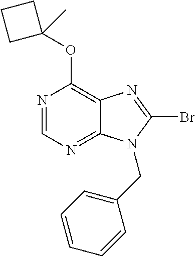 [SMILES: CC1(Oc2ncnc3c2nc(Br)n3Cc2ccccc2)CCC1]

Lithium bis(trimethylsilyl)amide (6.79 mL, 6.79 mmol) was added to a solution of 9-benzyl-6-(1-methylcyclobutoxy)-9H-purine (500 mg, 1.70 mmol) and 1,2-dibromo-1,1,2,2-tetrachloroethane (2.21 g, 6.79 mmol) in THF (30 mL) at 25° C. under nitrogen. The resulting solution was stirred at 25° C. for 5 hours. The reaction mixture was poured into water (150 mL) and extracted with EtOAc (3×100 mL). The organic layer was dried over Na₂SO₄, filtered and evaporated to afford the crude product. The crude product was purified by flash silica chromatography, elution gradient 0 to 30% EtOAc in petroleum ether. Pure fractions were evaporated to dryness to afford 9-benzyl-8-bromo-6-(1-methylcyclobutoxy)-9H-purine (400 mg, 63%) as a yellow gum. ¹H NMR (300 MHz, DMSO-d₆): 1.63 (3H, s), 1.67-1.87 (2H, m), 2.16-2.35 (2H, m), 2.37-2.40 (2H, m), 5.32 (2H, s), 7.11-7.16 (2H, m), 7.12-7.36 (3H, m), 8.37 (1H, s). m/z: ES+[M+H]+ 373.

### Synthetic Example 13

### 1-((9-Benzyl-8-(2-chloro-4-(2-(piperazin-1-yl)ethoxy)phenyl)-9H-purin-6-yl)oxy)cyclopropane-1-carbonitrile

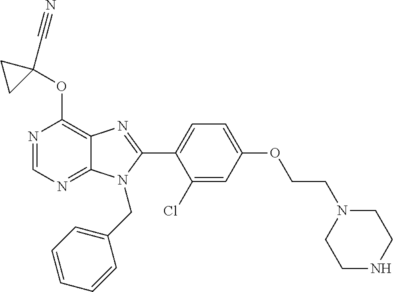 [SMILES: N#CC1(Oc2ncnc3c2nc(-c2ccc(OCCN4CCNCC4)cc2Cl)n3Cc2ccccc2)CC1]

Hydrogen chloride (4M in dioxane) (7.93 μl, 0.03 mmol) was added to a stirred solution of tert-butyl 4-(2-(4-(9-benzyl-6-(1-cyanocyclopropoxy)-9H-purin-8-yl)-3-chlorophenoxy)ethyl)piperazine-1-carboxylate (10 mg, 0.02 mmol) in anhydrous acetonitrile (0.5 mL) cooled to 0° C. After 20 minutes, additional hydrogen chloride (4M in dioxane, 7.9 μL, 0.03 mmol) was added and the reaction was allowed to warm to room temperature. Then the reaction mixture was diluted with methanol and the crude product was purified by ion exchange chromatography, using an SCX column. The desired product was eluted from the column using 1 M NH₃/MeOH and pure fractions were evaporated to dryness. The crude product was purified by preparative HPLC (Waters CSH C18 OBD column, 5 μm silica, 30 mm diameter, 100 mm length), using decreasingly polar mixtures of water (containing 1% concentrated aqueous ammonia) and MeCN as eluents. Fractions containing the desired compound were evaporated to dryness to afford 1-((9-benzyl-8-(2-chloro-4-(2-(piperazin-1-yl)ethoxy)phenyl)-9H-purin-6-yl)oxy)cyclopropane-1-carbonitrile (2.6 mg, 31%) as a white solid. ¹H NMR (500 MHz, CDCl₃): 1.52-1.63 (2H, m), 1.63-1.74 (2H, m), 2.68-2.75 (4H, m), 2.87 (2H, t), 3.02-3.1 (4H, m), 4.15 (2H, t), 5.36 (2H, s), 6.82 (1H, dd), 6.93 (2H, dd), 7.06 (1H, d), 7.14-7.23 (4H, m), 8.74 (1H, s)-one H not observed. m/z: ES+[M+H]+ 530.

Tert-butyl 4-(2-(4-(9-benzyl-6-(1-cyanocyclopropoxy)-9H-purin-8-yl)-3-chlorophenoxy)ethyl)piperazine-1-carboxylate used as starting material was made as follows:

### Tert-butyl 4-(2-(4-(9-benzyl-6-(1-cyanocyclopropoxy)-9H-purin-8-yl)-3-chlorophenoxy)ethyl)piperazine-1-carboxylate

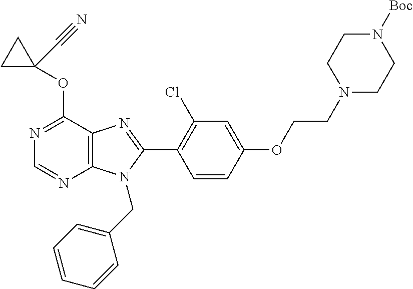 [SMILES: CC(C)(C)OC(=O)N1CCN(CCOc2ccc(-c3nc4c(OC5(C#N)CC5)ncnc4n3Cc3ccccc3)c(Cl)c2)CC1]

Sodium hydride (206 mg, 5.14 mmol) was added to tert-butyl 4-(2-(4-(9-benzyl-6-chloro-9H-purin-8-yl)-3-chlorophenoxy)ethyl)piperazine-1-carboxylate (150 mg, 0.26 mmol, Synthetic Example 6 starting material) in THF (6 mL) at 0° C. under nitrogen. 1-Hydroxycyclopropane-1-carbonitrile (107 mg, 1.29 mmol) was added after the reaction had been stirred for 15 minutes. The resulting solution was stirred at rt for a further 2 hours. The reaction mixture was poured into saturated NH₄Cl (75 mL) and extracted with EtOAc (3×100 mL). The organic layer was dried over Na₂SO₄, filtered and evaporated to afford a brown oil. The crude product was purified by flash silica chromatography, elution gradient 0 to 10% MeOH in DCM. Pure fractions were evaporated to dryness to afford tert-butyl 4-(2-(4-(9-benzyl-6-(1-cyanocyclopropoxy)-9H-purin-8-yl)-3-chlorophenoxy)ethyl)piperazine-1-carboxylate (73.0 mg, 45%) as a green oil. ¹H NMR (300 MHz, DMSO-d₆): 1.40 (13H, s), 1.64 (2H, t), 1.73-1.82 (2H, m), 2.45 (4H, t), 2.72-2.77 (2H, m), 4.21 (2H, t), 5.35 (2H, s), 6.89-6.94 (2H, m), 7.02-7.11 (1H, m), 7.17-7.24 (4H, m), 7.39-7.46 (1H, m), 8.78 (1H, s). m/z: ES+[M+H]+ 630.

### Synthetic Example 14

### 2-(2-Chloro-4-(2-(piperazin-1-yl)ethoxy)phenyl)-3-((4-chloropyridin-2-yl)methyl)-7-(1-methylcyclopropoxy)-3H-imidazo[4,5-b]pyridine

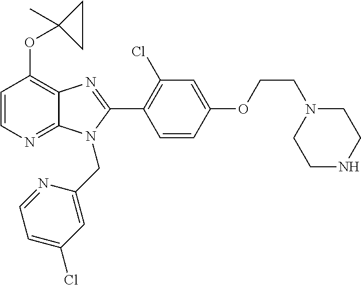 [SMILES: CC1(Oc2ccnc3c2nc(-c2ccc(OCCN4CCNCC4)cc2Cl)n3Cc2cc(Cl)ccn2)CC1]

p-Toluenesulfonic acid monohydrate (94 mg, 0.49 mmol) was added to N2-((4-chloropyridin-2-yl)methyl)-4-(1-methylcyclopropoxy)pyridine-2,3-diamine (100 mg, 0.33 mmol) and 2-chloro-4-(2-(piperazin-1-yl)ethoxy)benzaldehyde (106 mg, 0.39 mmol) in EtOH (5.5 mL) at 25° C. The resulting mixture was stirred at 60° C. for 2 hours. The reaction mixture was quenched with water (15 mL) and extracted with EtOAc (3×50 mL). The organic layer was dried over Na₂SO₄, filtered and evaporated to afford a brown oil. 21 Fractions containing the desired compound were evaporated to dryness to afford 2-(2-chloro-4-(2-(piperazin-1-yl)ethoxy)phenyl)-3-((4-chloropyridin-2-yl)methyl)-7-(1-methylcyclopropoxy)-3H-imidazo[4,5-b]pyridine (25 mg, 14%) as a white solid. ¹H NMR (400 MHz, CDCl₃): 0.84 (2H, t), 1.23 (2H, t), 1.72 (3H, s), 2.56-2.61 (4H, m), 2.81 (2H, t), 2.96 (4H, t), 4.12 (2H, t), 5.49 (2H, s), 6.76-6.85 (2H, m), 7.01 (1H, d), 7.06-7.13 (2H, m), 7.30 (1H, d), 8.31 (2H₂, t)-one proton not observed. m/z: ES+[M+H]+ 553.

2-Chloro-4-(2-(piperazin-1-yl)ethoxy)benzaldehyde used as starting material was made as follows:

### 2-Chloro-4-(2-(piperazin-1-yl)ethoxy)benzaldehyde

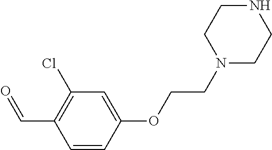 [SMILES: O=Cc1ccc(OCCN2CCNCC2)cc1Cl]

2,2,2-trifluoroacetic acid (3 mL, 2.71 mmol) was added to tert-butyl 4-(2-(3-chloro-4-formylphenoxy)ethyl)piperazine-1-carboxylate (1.0 g, 2.71 mmol, Synthetic Example 6 starting material) in DCM (5 mL) at 25° C. The resulting mixture was stirred at 25° C. for 1 hour. The reaction mixture was diluted with EtOAc (25 mL) and washed sequentially with water (3×25 mL). The organic layer was dried over Na₂SO₄, filtered and evaporated to afford the crude product. The crude product was purified by flash silica chromatography, elution gradient 0 to 50% pentane in EtOAc. Pure fractions were evaporated to dryness to afford 2-chloro-4-(2-(piperazin-1-yl)ethoxy)benzaldehyde (0.700 g, 96%) as a yellow gum. ¹H NMR (300 MHz, DMSO-d₆): 3.22-3.39 (10H, m), 4.41 (2H, t), 7.11 (1H, dd), 7.23 (1H, d), 7.86 (1H, d), 10.20 (1H, d). One H not observed. m/z: ES+[M+H]+ 269.

N2-((4-Chloropyridin-2-yl)methyl)-4-(1-methylcyclopropoxy)pyridine-2,3-diamine used as starting material was made as follows:

### 2-Chloro-4-(1-methylcyclopropoxy)-3-nitropyridine

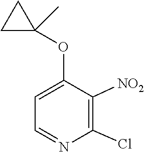

Sodium hydride (0.415 g, 10.4 mmol) was added in one portion to 2,4-dichloro-3-nitropyridine (2 g, 10.4 mmol) and 1-methylcyclopropan-1-ol (0.747 g, 10.4 mmol) in THF (20 mL) at 0° C. under nitrogen. The resulting suspension was stirred at 25° C. for 1 hour. The reaction mixture was quenched with water (20 mL), extracted with EtOAc (3×50 mL). The top layer was dried over Na₂SO₄, filtered and evaporated to afford a yellow solid, which was purified by preparative TLC (petroleum ether/EtOAc 3:1), to afford 2-chloro-4-(1-methylcyclopropoxy)-3-nitropyridine (0.900 g, 38%) as a yellow solid. ¹H NMR (300 MHz, DMSO-d₆): 0.85-0.92 (2H, m), 0.98-1.05 (2H, m), 1.57 (3H, s), 7.66 (1H, d), 8.51 (1H, d). m/z: ES+[M+H]+ 229.

N-((4-chloropyridin-2-yl)methyl)-4-(1-methylcyclopropoxy)-3-nitropyridin-2-amine

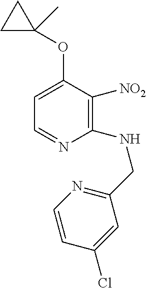 [SMILES: CC1(Oc2ccnc(NCc3cc(Cl)ccn3)c2[N+](=O)[O-])CC1]

BINAP (82 mg, 0.13 mmol) was added to 2-chloro-4-(1-methylcyclopropoxy)-3-nitropyridine (300 mg, 1.31 mmol), (4-chloropyridin-2-yl)methanamine (281 mg, 1.97 mmol), Cₛ₂CO₃ (1283 mg, 3.94 mmol) and Pd₂(dba)₃ (120 mg, 0.13 mmol) in dioxane (7 mL) at 25° C. The resulting mixture was stirred at 100° C. for 2 hours. The reaction mixture was quenched with water (20 mL), extracted with EtOAc (3×100 mL) and washed sequentially with water (2×50 mL), saturated brine (2×50 mL). The organic layer was dried over Na₂SO₄, filtered and evaporated to afford a yellow oil. This oil was purified by preparative TLC (petroleum ether/EtOAc 2:1), to afford N-((4-chloropyridin-2-yl)methyl)-4-(1-methylcyclopropoxy)-3-nitropyridin-2-amine (275 mg, 63%) as a yellow solid. ¹H NMR (400 MHz, DMSO-d₆): 0.79-0.93 (2H, m), 0.93-1 (2H, m), 1.54 (3H, s), 4.69 (2H, d), 6.74 (1H, d), 7.36 (1H, d), 7.40 (1H, m), 7.87 (1H, t), 8.07 (1H, d), 8.49 (1H, d). m/z: ES+[M+H]+ 335.

### N2-((4-chloropyridin-2-yl)methyl)-4-(1-methylcyclopropoxy)pyridine-2,3-diamine

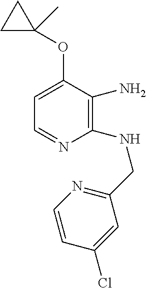 [SMILES: CC1(Oc2ccnc(NCc3cc(Cl)ccn3)c2N)CC1]

Iron (307 mg, 5.50 mmol) was added to N-((4-chloropyridin-2-yl)methyl)-4-(1-methylcyclopropoxy)-3-nitropyridin-2-amine (230 mg, 0.69 mmol) and ammonium chloride (294 mg, 5.50 mmol) in EtOH:H₂O (4:1) (8 mL) at 25° C. The resulting mixture was stirred at 80° C. for 2 hours. The mixture was filtered through a celite pad and the solvent was removed. The reaction mixture was quenched with water (15 mL), extracted with EtOAc (3×50 mL), and washed sequentially with water (2×20 mL) and saturated brine (2×20 mL). The organic layer was dried over Na₂SO₄, filtered and evaporated to afford a yellow oil. This oil was purified by preparative TLC (EtOAc) to afford N2-((4-chloropyridin-2-yl)methyl)-4-(1-methylcyclopropoxy)pyridine-2,3-diamine (110 mg, 52%) as a yellow oil. ¹H NMR (300 MHz, DMSO-d₆): 0.76 (2H, t), 0.81-0.96 (2H, m), 1.50 (3H, s), 4.14 (2H, s), 4.63 (2H, d), 6.25 (1H, t), 6.53 (1H, d), 7.29-7.42 (3H, m), 8.48 (1H, d). m/z: ES+[M+H]+ 305.

### Synthetic Example 15

### 2-(2-chloro-4-(2-(piperazin-1-yl)ethoxy)phenyl)-1-((4-chloropyridin-2-yl)methyl)-4-(1-methylcyclopropoxy)-1H-benzo[d]imidazole

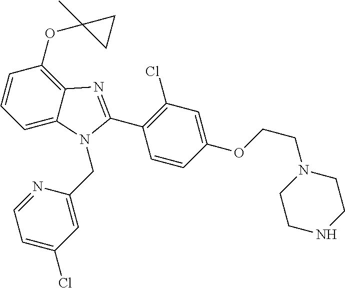 [SMILES: CC1(Oc2cccc3c2nc(-c2ccc(OCCN4CCNCC4)cc2Cl)n3Cc2cc(Cl)ccn2)CC1]

2,2,2-Trifluoroacetic acid (0.3 mL, 3.9 mmol) was added to tert-butyl 4-(2-(3-chloro-4-(1-((4-chloropyridin-2-yl)methyl)-4-(1-methylcyclopropoxy)-1H-benzo[d]imidazol-2-yl)phenoxy)ethyl)piperazine-1-carboxylate (80 mg, 0.12 mmol) in DCM (1 mL) at 25° C. The resulting mixture was stirred at 25° C. for 1 hour. The reaction mixture was diluted with EtOAc (10 mL) and washed sequentially with water (3×10 mL). The organic layer was dried over Na₂SO₄, filtered and evaporated to afford the crude product. The crude product was purified by preparative HPLC (XBridge Shield RP18 OBD column, 15*150 mm, 10 μm), using decreasingly polar mixtures of water (containing 10 mmol/L NH₄HCO₃ and 0.1% aqueous ammonia) and MeCN as eluents. Fractions containing the desired compound were evaporated to dryness to afford 2-(2-chloro-4-(2-(piperazin-1-yl)ethoxy)phenyl)-1-((4-chloropyridin-2-yl)methyl)-4-(1-methylcyclopropoxy)-1H-benzo[d]imidazole (28 mg, 41%) as a white solid. ¹H NMR (400 MHz, DMSO-d₆): 0.82 (2H, t), 0.98 (2H, t), 1.59 (3H, s), 2.31-2.45 (4H, m), 2.6-2.76 (6H, m), 4.15 (2H, t), 5.34 (2H, s), 6.95-7.02 (2H, m), 7.06 (1H, d), 7.13-7.19 (2H, m), 7.21 (1H, d), 7.37-7.43 (2H, m), 8.36 (1H, d)-one H not observed. m/z: ES+[M+H]+ 552.

Tert-butyl 4-(2-(3-chloro-4-(1-((4-chloropyridin-2-yl)methyl)-4-(1-methylcyclopropoxy)-1H-benzo[d]imidazol-2-yl)phenoxy)ethyl)piperazine-1-carboxylate used as starting material was made as follows:

### 1-Fluoro-3-(1-methylcyclopropoxy)-2-nitrobenzene

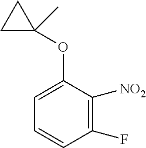

Sodium hydride (78 mg, 3.27 mmol) was added to 1,3-difluoro-2-nitrobenzene (400 mg, 2.51 mmol) and 1-methylcyclopropan-1-ol (181 mg, 2.51 mmol) in THF (3 mL) at 0° C. The resulting mixture was stirred at 25° C. for 2 hours. The reaction mixture was concentrated, diluted with EtOAc (20 mL) and washed sequentially with water (3×5 mL) followed by saturated brine (3×5 mL). The top layer was dried over Na₂SO₄, filtered and evaporated to afford the crude product. The crude product was purified by preparative TLC (petroleum ether/EtOAc 2:1), to afford 1-fluoro-3-(1-methylcyclopropoxy)-2-nitrobenzene (440 mg, 83%) as a yellow solid. ¹H NMR (300 MHz, DMSO-d₆): 0.79-1.01 (4H, m), 1.54 (3H, s), 7.15-7.19 (1H, m), 7.35-7.39 (1H, m), 7.63-7.65 (1H, m). m/z: ES+[M+H]+ 212.

### N-((4-chloropyridin-2-yl)methyl)-3-(1-methylcyclopropoxy)-2-nitroaniline

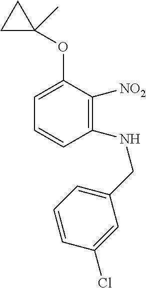 [SMILES: CC1(Oc2cccc(NCc3cccc(Cl)c3)c2[N+](=O)[O-])CC1]

N,N-Diisopropylethylamine (826 mg, 6.39 mmol) was added to 1-fluoro-3-(1-methylcyclopropoxy)-2-nitrobenzene (270 mg, 1.28 mmol) and (4-chloropyridin-2-yl)methanamine (219 mg, 1.53 mmol) in DMSO (5 mL) at 25° C. The resulting mixture was stirred at 130° C. for 2 hours. The reaction mixture was concentrated, diluted with EtOAc (20 mL) and washed sequentially with water (3×5 mL) and saturated brine (3×5 mL). The top layer was dried over Na₂SO₄, filtered and evaporated to afford the crude product. The crude product was purified by flash silica chromatography, elution gradient 0 to 5% MeOH in DCM. Pure fractions were evaporated to dryness to afford N-((4-chloropyridin-2-yl)methyl)-3-(1-methylcyclopropoxy)-2-nitroaniline (237 mg, 55%) as a yellow solid. ¹H NMR (300 MHz, CDCl₃): 0.73 (2H, t), 1.06 (2H, t), 1.58 (3H, s), 4.56 (2H, s), 6.24 (1H, dd), 6.68 (1H, dd), 7.18 (1H, t), 7.23-7.26 (1H, m), 7.27-7.29 (1H, m), 7.36 (1H, d), 8.50 (1H, d). m/z: ES+[M+H]+ 334.

### Tert-butyl 4-(2-(3-chloro-4-(1-((4-chloropyridin-2-yl)methyl)-4-(1-methylcyclopropoxy)-1H-benzo[d]imidazol-2-yl)phenoxy)ethyl)piperazine-1-carboxylate

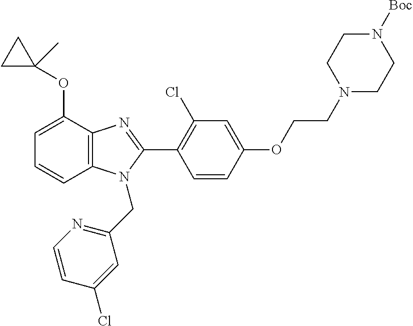 [SMILES: CC(C)(C)OC(=O)N1CCN(CCOc2ccc(-c3nc4c(OC5(C)CC5)cccc4n3Cc3cc(Cl)ccn3)c(Cl)c2)CC1]

Sodium hydrosulfite (730 mg, 4.19 mmol) was added to N-((4-chloropyridin-2-yl)methyl)-3-(1-methylcyclopropoxy)-2-nitroaniline (140 mg, 0.42 mmol) in MeOH (2 mL) and water (2 mL) at ° C. The mixture was stirred for 16 hours. Then tert-butyl 4-(2-(3-chloro-4-formylphenoxy)ethyl)piperazine-1-carboxylate (201 mg, 0.55 mmol) and 4-methylbenzenesulfonic acid (87 mg, 0.50 mmol) was added. The reaction mixture was stirred at 25° C. for a further 3 hours. The reaction mixture was concentrated and then diluted with EtOAc (15 mL). The crude mixture was then washed sequentially with water (3×15 mL). The organic layer was dried over Na₂SO₄, filtered and evaporated to afford the crude product. The crude product was purified by flash silica chromatography, elution gradient 0 to 10% MeOH in DCM. Pure fractions were evaporated to dryness to afford tert-butyl 4-(2-(3-chloro-4-(1-((4-chloropyridin-2-yl)methyl)-4-(1-methylcyclopropoxy)-1H-benzo[d]imidazol-2-yl)phenoxy)ethyl)piperazine-1-carboxylate (90 mg, 33%) as a yellow gum. ¹H NMR (300 MHz, DMSO-d₆): 0.80 (2H, t), 0.97 (2H, t), 1.38 (9H, s), 1.57 (3H, s), 2.38-2.47 (4H, m), 2.72 (2H, t), 3.25-3.31 (4H, m), 4.16 (2H, t), 5.33 (2H, s), 6.93-7.08 (3H, m), 7.11-7.22 (3H, m), 7.35-7.42 (2H, m), 8.35 (1H, d). m/z: ES+[M+H]+ 652.

### Synthetic Example 16

### 2-(2-chloro-4-(2-(piperazin-1-yl)ethoxy)phenyl)-1-((4-chloropyridin-2-yl)methyl)-4-(1-methylcyclopropoxy)-1H-imidazo[4,5-c]pyridine

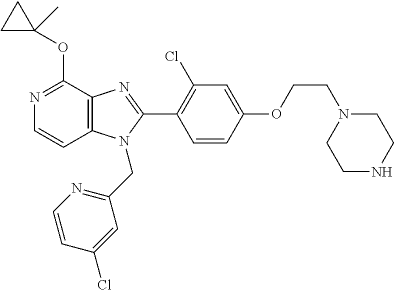 [SMILES: CC1(Oc2nccc3c2nc(-c2ccc(OCCN4CCNCC4)cc2Cl)n3Cc2cc(Cl)ccn2)CC1]

HCl (4M in dioxane, 123 mg, 3.37 mmol) was added to tert-butyl 4-(2-(3-chloro-4-(1-((4-chloropyridin-2-yl)methyl)-4-(1-methylcyclopropoxy)-1H-imidazo[4,5-c]pyridin-2-yl)phenoxy)ethyl)piperazine-1-carboxylate (220 mg, 0.34 mmol) in dichloromethane (1 mL) at rt.

The reaction mixture was stirred at rt for 1 hour, then evaporated to dryness, redissolved in DMF (2 mL) and filtered through celite. The residue was purified by preparative HPLC (YMC-Actus Triart C18 ExRS column, 30*150 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness to afford 2-(2-chloro-4-(2-(piperazin-1-yl)ethoxy)phenyl)-1-((4-chloropyridin-2-yl)methyl)-4-(1-methylcyclopropoxy)-1H-imidazo[4,5-c]pyridine (67 mg, 36%) as a white solid. ¹H NMR (400 MHz, DMSO-d₆): 0.78 (2H, t), 0.93 (2H, t), 1.69 (3H, s), 2.34-2.44 (4H, m), 2.59-2.76 (6H, m), 4.15 (2H, t), 5.38 (2H, s), 6.95-7.02 (1H, m), 7.19-7.24 (3H, m), 7.34-7.42 (2H, m), 7.92 (1H, d), 8.34 (1H, d)-one proton not observed. m/z: ES+[M+H]+ 553

Tert-butyl 4-(2-(3-chloro-4-(1-((4-chloropyridin-2-yl)methyl)-4-(1-methylcyclopropoxy)-1H-imidazo[4,5-c]pyridin-2-yl)phenoxy)ethyl)piperazine-1-carboxylate used as starting material was made as follows:

### 2-Chloro-4-(methylthio)-3-nitropyridine

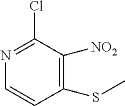

Sodium thiomethoxide (0.872 g, 12.4 mmol) was added to 2,4-dichloro-3-nitropyridine (2 g, 10.4 mmol) in MeOH (10 mL) and the reaction mixture was stirred at rt for 16 hours. The reaction mixture was evaporated to dryness, redissolved in EtOAc (50 mL), and washed sequentially with water (2×15 mL), saturated brine (2×15 mL). The organic layer was dried over Na₂SO₄, filtered and evaporated to afford the crude product. The crude product was purified by flash silica chromatography, elution gradient 0 to 30% EtOAc in petroleum ether. Pure fractions were evaporated to dryness to afford 2-chloro-4-(methylthio)-3-nitropyridine (1.40 g, 66%) as a yellow solid. ¹H NMR (300 MHz, DMSO-d₆): 2.40 (3H, s), 7.54 (1H, d), 8.37 (1H, d). m/z: ES+[M+H]+ 205.

### 2-(1-Methylcyclopropoxy)-4-(methylthio)-3-nitropyridine

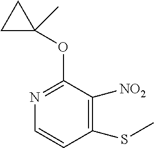 [SMILES: CSc1ccnc(OC2(C)CC2)c1[N+](=O)[O-]]

Sodium hydride (2.06 g, 86.0 mmol) was added in one portion to 2-chloro-4-(methylthio)-3-nitropyridine (2.2 g, 10.7 mmol) and 1-methylcyclopropan-1-ol (1.16 g, 16.1 mmol) in THF (200 mL) at 0° C. under nitrogen. The resulting suspension was stirred at rt for 6 hours. Then the reaction mixture was diluted with EtOAc (100 mL), and washed with saturated brine (2×50 mL). The organic layer was dried over Na₂SO₄, filtered and evaporated to afford the crude product. The crude product was purified by flash silica chromatography, elution gradient 10 to 40% EtOAc in petroleum ether. Pure fractions were evaporated to dryness to afford 2-(1-methylcyclopropoxy)-4-(methylthio)-3-nitropyridine (1.30 g, 50%) as a brown solid. ¹H NMR (300 MHz, DMSO-d₆): 0.76 (2H, t), 0.88 (2H, t), 1.62 (3H, s), 2.59 (3H, s), 7.21 (1H, d), 8.29 (1H, d). m/z: ES+[M+H]+ 241.

### 2-(1-Methylcyclopropoxy)-4-(methylsulfonyl)-3-nitropyridine

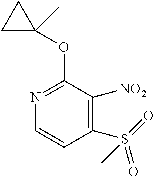 [SMILES: CC1(Oc2nccc(S(C)(=O)=O)c2[N+](=O)[O-])CC1]

m-Chloroperbenzoic acid (2.05 g, 11.9 mmol) was added to 2-(1-methylcyclopropoxy)-4-(methylthio)-3-nitropyridine (1.30 g, 5.41 mmol) in dichloromethane (100 mL) and the reaction was stirred at rt for 6 hours. Then the reaction mixture was evaporated to dryness and redissolved in EtOAc (100 mL) and washed sequentially with water (3×50 mL) and saturated brine (3×50 mL). The organic layer was dried over Na₂SO₄, filtered and evaporated to afford the crude product. The crude product was purified by flash silica chromatography, elution gradient 25 to 50% EtOAc in petroleum ether. Pure fractions were evaporated to dryness to afford 2-(1-methylcyclopropoxy)-4-(methylsulfonyl)-3-nitropyridine (720 mg, 49%) as a yellow solid. ¹H NMR (300 MHz, DMSO-d6): 0.80 (2H, t), 0.93 (2H, t), 1.64 (3H, s), 3.46 (3H, s), 7.69 (1H, d), 8.78 (1H, d). m/z: ES+[M+H]+ 273.

### N-((4-chloropyridin-2-yl)methyl)-2-(1-methylcyclopropoxy)-3-nitropyridin-4-amine

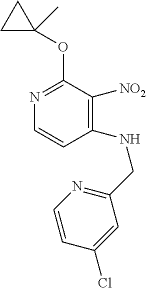 [SMILES: CC1(Oc2nccc(NCc3cc(Cl)ccn3)c2[N+](=O)[O-])CC1]

Triethylamine (0.530 mL, 3.80 mmol) was added to 4-chloro-2-pyridinemethanamine (542 mg, 3.80 mmol) and 2-(1-methylcyclopropoxy)-4-(methylsulfonyl)-3-nitropyridine (690 mg, 2.53 mmol) in DMF (110 mL). The reaction mixture was stirred at 60° C. for 16 hours. Then the reaction mixture was cooled, concentrated and further diluted with EtOAc (100 mL). The organic layer was washed sequentially with water (2×75 mL) and saturated brine (2×75 mL), dried over Na₂SO₄, filtered and evaporated. The crude product was purified by flash silica chromatography, elution gradient 30 to 60% EtOAc in petroleum ether. Pure fractions were evaporated to dryness to afford N-((4-chloropyridin-2-yl)methyl)-2-(1-methylcyclopropoxy)-3-nitropyridin-4-amine (260 mg, 31%) as a yellow solid. ¹H NMR (300 MHz, DMSO-d₆): 0.72 (2H, t), 0.84 (2H, t), 1.60 (3H, s), 4.60 (2H, d), 6.46 (1H, d), 7.4-7.6 (2H, m), 7.86 (1H, d), 7.98 (1H, t), 8.44-8.61 (1H, m). m/z: ES+[M+H]+ 335.

### N4-((4-chloropyridin-2-yl)methyl)-2-(1-methylcyclopropoxy)pyridine-3,4-diamine

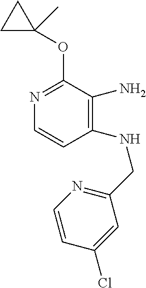 [SMILES: CC1(Oc2nccc(NCc3cc(Cl)ccn3)c2N)CC1]

Iron (417 mg, 7.47 mmol) was added to ammonium chloride (40 mg, 0.75 mmol) and N-((4-chloropyridin-2-yl)methyl)-2-(1-methylcyclopropoxy)-3-nitropyridin-4-amine (250 mg, 0.75 mmol) in ethanol/water (10:1; 1 mL). The reaction mixture was stirred at 60° C. for 3 hours. The reaction mixture then cooled, evaporated to dryness and redissolved in EtOAc (200 mL). The organic layer was washed sequentially with water (2×100 mL) and saturated brine (2×100 mL), dried over Na₂SO₄, filtered and evaporated to afford the crude product. The crude product was purified by flash silica chromatography, elution gradient 30 to 60% EtOAc in petroleum ether. Pure fractions were evaporated to dryness to afford N4-((4-chloropyridin-2-yl)methyl)-2-(1-methylcyclopropoxy)pyridine-3,4-diamine (170 mg, 75%) as a yellow solid. ¹H NMR (300 MHz, DMSO-d₆): 0.64 (2H, t), 0.82 (2H, t), 1.58 (3H, s), 3.88-4.16 (2H, m), 4.45 (2H, d), 6.07 (2H, d), 7.23 (1H, d), 7.38-7.53 (2H, m), 8.42-8.66 (1H, m). m/z: ES+[M+H]+ 305.

### Tert-butyl 4-(2-(3-chloro-4-(1-((4-chloropyridin-2-yl)methyl)-4-(1-methylcyclopropoxy)-1H-imidazof4,5-c]pyridin-2-yl)phenoxy)ethyl)piperazine-1-carboxylate

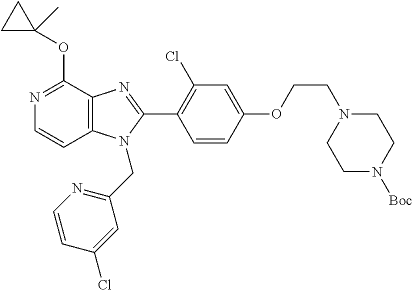 [SMILES: CC(C)(C)OC(=O)N1CCN(CCOc2ccc(-c3nc4c(OC5(C)CC5)nccc4n3Cc3cc(Cl)ccn3)c(Cl)c2)CC1]

Tert-butyl 4-(2-(3-chloro-4-formylphenoxy)ethyl)piperazine-1-carboxylate (203 mg, 0.55 mmol) was added to N4-((4-chloropyridin-2-yl)methyl)-2-(1-methylcyclopropoxy)pyridine-3,4-diamine (140 mg, 0.46 mmol) in MeOH (20 mL) and acetic acid (1 mL) was stirred at rt for 16 hours. The reaction mixture was evaporated to dryness, redissolved in EtOAc (100 mL), and washed sequentially with water (2×50 mL) and saturated brine (2×50 mL). The organic layer was dried over Na₂SO₄, filtered and evaporated to afford the crude product. The crude product was purified by flash silica chromatography, elution gradient 30 to 70% EtOAc in petroleum ether. Pure fractions were evaporated to dryness to afford tert-butyl 4-(2-(3-chloro-4-(1-((4-chloropyridin-2-yl)methyl)-4-(1-methylcyclopropoxy)-1H-imidazo[4,5-c]pyridin-2-yl)phenoxy)ethyl)piperazine-1-carboxylate (230 mg, 77%) as a brown solid. ¹H NMR (300 MHz, DMSO-d₆): 0.78 (2H, t), 0.94 (2H, t), 1.40 (9H, s), 1.70 (3H, s), 2.41-2.47 (4H, m), 2.74 (2H, t), 3.25-3.4 (4H, m), 4.18 (2H, t), 5.39 (2H, s), 6.97-7.04 (1H, m), 7.2-7.25 (3H, m), 7.35-7.43 (2H, m), 7.93 (1H, d), 8.35 (1H, d). m/z: ES+[M+H]+ 653.

### Synthetic Example 17

### 3-(3-Chloro-4-(9-((4-chloropyridin-2-yl)methyl)-6-(1-methylcyclopropoxy)-9H-purin-8-yl)phenoxy)-N,N-dimethylpropan-1-amine

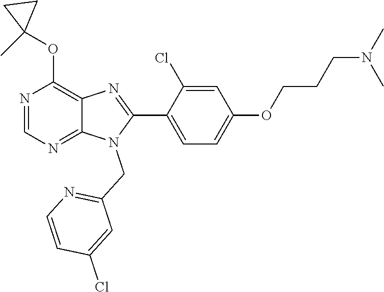

K₂CO₃ (56 mg, 0.41 mmol) was added to 3-chloro-4-(9-((4-chloropyridin-2-yl)methyl)-6-(1-methylcyclopropoxy)-9H-purin-8-yl)phenol (60 mg, 0.14 mmol) and 3-bromo-N,N-dimethylpropan-1-amine (45 mg, 0.27 mmol) in DMF (1 mL). The resulting mixture was stirred at 60° C. for 4 hours. The reaction mixture was filtered through celite. The crude filtrate was purified by preparative HPLC (XBridge Shield RP18 OBD column, 30*150 mm, 5 μm), using decreasingly polar mixtures of water (containing 10 mmol/L NH₄HCO₃ and 0.1% aqueous ammonia) and MeCN as eluents. The fractions containing the desired compound were evaporated to dryness to afford 3-(3-chloro-4-(9-((4-chloropyridin-2-yl)methyl)-6-(1-methylcyclopropoxy)-9H-purin-8-yl)phenoxy)-N,N-dimethylpropan-1-amine (8.0 mg, 11%) as a yellow solid. ¹H NMR (400 MHz, DMSO-d₆): 0.85 (2H, t), 1.02 (2H, t), 1.73 (3H, s), 1.79-1.91 (2H, m), 2.13 (6H, s), 2.34 (2H, t), 4.07 (2H, t), 5.40 (2H, s), 6.96 (1H, dd), 7.17 (1H, d), 7.26 (1H, d), 7.32-7.44 (2H, m), 8.30 (1H, d), 8.57 (1H, s). m/z: ES+[M+H]+ 527.

3-Chloro-4-(9-((4-chloropyridin-2-yl)methyl)-6-(1-methylcyclopropoxy)-9H-purin-8-yl)phenol used as starting material was made as follows:

### 4-((Tert-butyldiphenylsilyl)oxy)-2-chlorobenzaldehyde

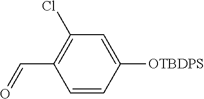

Tert-butylchlorodiphenylsilane (26.3 g, 95.8 mmol) was added to a mixture of 1H-imidazole (4.87 g, 71.5 mmol) and 2-chloro-4-hydroxybenzaldehyde (10 g, 63.9 mmol) in DMF (200 mL) at rt. The mixture was stirred at this temperature for 3 hours. Then the reaction mixture was diluted with EtOAc (500 mL) and washed sequentially with water (3×400 mL) and saturated brine (2×400 mL). The organic layer was dried over Na₂SO₄, filtered and evaporated to afford the crude product. The crude product was purified by flash silica chromatography, elution gradient 0 to 30% EtOAc in petroleum ether. Pure fractions were evaporated to dryness to afford 4-((tert-butyldiphenylsilyl)oxy)-2-chlorobenzaldehyde (13.0 g, 51%) as a white solid. ¹H NMR (300 MHz, DMSO-d₆): 1.01 (9H, dd), 6.89-6.95 (1H, m), 7.33-7.56 (7H, m), 7.64-7.72 (5H, m), 10.14 (1H, dd). m/z: ES+[M+H]+ 395.

### 8-(4-((Tert-butyldiphenylsilyl)oxy)-2-chlorophenyl)-6-chloro-9H-purine

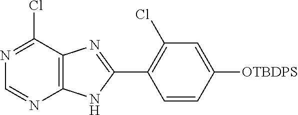

Iron(III) chloride solution (51.3 g, 316 mmol) was added to 6-chloropyrimidine-4,5-diamine (21.96 g, 151.9 mmol) and 4-((tert-butyldiphenylsilyl)oxy)-2-chlorobenzaldehyde (50 g, 126.6 mmol) in IPA (500 mL). The reaction mixture was stirred at 60° C. for 4 hours. The reaction mixture was adjusted to pH 7 with 1 M NaOH. The reaction mixture was poured into water (1.5 L), extracted with EtOAc (3×1.5 L). The organic layer was dried over Na₂SO₄, filtered and evaporated to afford the crude product. The crude product was purified by flash silica chromatography, elution gradient 0 to 20% EtOAc in petroleum ether. Pure fractions were evaporated to dryness to afford 8-(4-((tert-butyldiphenylsilyl)oxy)-2-chlorophenyl)-6-chloro-9H-purine (29.0 g, 44%) as a yellow solid. ¹H NMR (300 MHz, DMSO-d₆): 1.08 (9H, s), 6.86 (1H, dd), 7.03 (1H, d), 7.44-7.55 (7H, m), 7.69-7.75 (4H, m), 8.74 (1H, s), 14.06 (1H, s). m/z: ES+[M+H]+ 519.

### 8-(4-((Tert-butyldiphenylsilyl)oxy)-2-chlorophenyl)-6-chloro-9-((4-chloropyridin-2-yl)methyl)-9H-purine

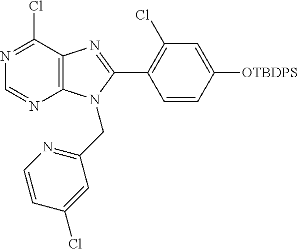

DIAD (9.36 mL, 48.1 mmol) was added to (4-chloropyridin-2-yl)methanol (3.32 g, 23.1 mmol), 8-(4-((tert-butyldiphenylsilyl)oxy)-2-chlorophenyl)-6-chloro-9H-purine (10 g, 19.2 mmol) and triphenylphosphine (12.62 g, 48.1 mmol) in THF (125 mL) at 0° C. over a period of 5 minutes under nitrogen. The resulting mixture was stirred at rt for 2 hours. The reaction mixture was poured into water (750 mL) and extracted with EtOAc (3×750 mL). The organic layer was dried over Na₂SO₄, filtered and evaporated to afford a brown oil. The crude product was purified by flash silica chromatography, elution gradient 0 to 30% EtOAc in petroleum ether. Pure fractions were evaporated to dryness to afford 8-(4-((tert-butyldiphenylsilyl)oxy)-2-chlorophenyl)-6-chloro-9-((4-chloropyridin-2-yl)methyl)-9H-purine (12.00 g, 97%) as a beige waxy solid. ¹H NMR (300 MHz, DMSO-d₆): 1.05 (9H, s), 5.45 (2H, s), 6.69 (1H, dd), 6.94 (1H, d), 7.27-7.33 (2H, m), 7.41-7.55 (7H, m), 7.63-7.69 (4H, m), 8.23 (1H, d), 8.79 (1H, s). m/z: ES+[M+H]+ 644.

### 3-Chloro-4-(9-((4-chloropyridin-2-yl)methyl)-6-(1-methylcyclopropoxy)-9H-purin-8-yl)phenol

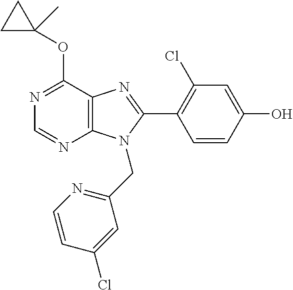 [SMILES: CC1(Oc2ncnc3c2nc(-c2ccc(O)cc2Cl)n3Cc2cc(Cl)ccn2)CC1]

Sodium hydride (2.60 g, 65.1 mmol) was added to solution of 8-(4-((tert-butyldiphenylsilyl)oxy)-2-chlorophenyl)-6-chloro-9-((4-chloropyridin-2-yl)methyl)-9H-purine (12 g, 18.6 mmol) in DMF (12 mL) at 0° C. under nitrogen. The reaction mixture was stirred for 15 minutes and then 1-methylcyclopropan-1-ol (4.19 g, 46.5 mmol) was added at 0° C. The resulting solution was allowed to warm up to rt and stirred for 2 hours. The reaction mixture was then poured into a solution of aqueous saturated NH₄Cl (500 mL) and extracted with EtOAc (3×500 mL). The organic layer was dried over Na₂SO₄, filtered and evaporated to afford the crude product as a brown oil. The crude product was purified by flash silica chromatography, elution gradient 50 to 70% EtOAc in petroleum ether. Pure fractions were evaporated to dryness to afford 3-chloro-4-(9-((4-chloropyridin-2-yl)methyl)-6-(1-methylcyclopropoxy)-9H-purin-8-yl)phenol (3.00 g, 36%) as a white solid. ¹H NMR (300 MHz, DMSO-d₆): 0.84 (2H, t), 1.02 (2H, t), 1.73 (3H, s), 5.41 (2H, s), 6.78 (1H, dd), 6.94 (1H, d), 7.21-7.32 (2H, m), 7.37 (1H, dd), 8.31 (1H, d), 8.56 (1H, s), 10.43 (1H, s). m/z: ES+[M+H]+ 442.

### Synthetic Example 18

### 8-(4-(2-(2,6-Diazaspiro[3.3]heptan-2-yl)ethoxy)-2-chlorophenyl)-9-benzyl-6-(1-methylcyclopropoxy)-9H-purine

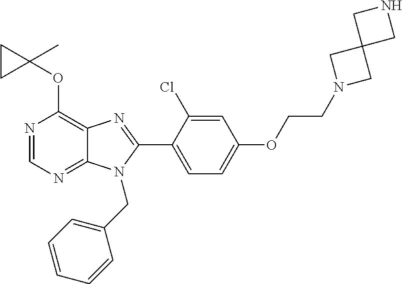 [SMILES: CC1(Oc2ncnc3c2nc(-c2ccc(OCCN4CC5(CNC5)C4)cc2Cl)n3Cc2ccccc2)CC1]

To tert-butyl 6-(2-(4-(9-benzyl-6-(1-methylcyclopropoxy)-9H-purin-8-yl)-3-chlorophenoxy)ethyl)-2,6-diazaspiro[3.3]heptane-2-carboxylate (150 mg, 0.24 mmol) was added TFA (3 mL) at rt. The resulting mixture was stirred at 25° C. for 30 minutes. The reaction mixture was diluted with DCM (50 mL) and neutralised with aqueous saturated NaHCO₃. The organic layer was further washed with saturated brine (3×10 mL), dried over Na₂SO₄, filtered and evaporated to afford the crude product. The crude product was purified by preparative HPLC (XBridge Prep OBD C18 column, 30*150 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness to afford 8-(4-(2-(2,6-diazaspiro[3.3]heptan-2-yl)ethoxy)-2-chlorophenyl)-9-benzyl-6-(1-methylcyclopropoxy)-9H-purine (29.3 mg, 23%) as a yellow solid. ¹H NMR (400 MHz, CDCl₃): 0.84 (2H, t), 1.16 (2H, t), 1.81 (3H, s), 2.83 (2H, t), 3.36-3.48 (4H, m), 3.83 (4H, s), 4.01 (2H, t), 5.35 (2H, s), 6.80 (1H, dd), 6.91-6.96 (2H, m), 7.04 (1H, d), 7.14-7.21 (4H, m), 8.68 (1H, s)-one H not observed. m/z: ES+[M+H]+ 531.

Tert-butyl 6-(2-(4-(9-benzyl-6-(1-methylcyclopropoxy)-9H-purin-8-yl)-3-chlorophenoxy)ethyl)-2,6-diazaspiro[3.3]heptane-2-carboxylate used as starting material was made as follows:

### 4-(2-Bromoethoxy)-2-chlorobenzaldehyde

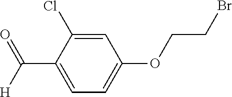 [SMILES: O=Cc1ccc(OCCBr)cc1Cl]

1,2-Dibromoethane (120 g, 639 mmol) was added to 2-chloro-4-hydroxybenzaldehyde (10 g, 63.9 mmol) and K₂CO₃ (22.07 g, 160 mmol) in MeCN (50 mL) at 25° C. The resulting mixture was stirred at 60° C. for 8 hours. The reaction mixture was filtered through a filtration paper and concentrated in vacuo. The crude product was purified by flash silica chromatography, elution gradient 0 to 40% EtOAc in petroleum ether. Pure fractions were evaporated to dryness to afford 4-(2-bromoethoxy)-2-chlorobenzaldehyde (13.00 g, 77%) as a white solid. ¹H NMR (300 MHz, DMSO-d₆): 3.82 (2H, t), 4.48 (2H, t), 7.11 (1H, dd), 7.23 (1H, d), 7.83 (1H, d), 10.19 (1H, s). m/z: ES+[M+H]+ 265 (³⁵Cl⁸¹Br/³⁷C⁷⁹Br peak).

### Tert-butyl 6-(2-(3-chloro-4-formylphenoxy)ethyl)-2,6-diazaspiro[3.3]heptane-2-carboxylate

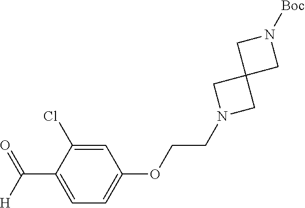 [SMILES: CC(C)(C)OC(=O)N1CC2(CN(CCOc3ccc(C=O)c(Cl)c3)C2)C1]

4-(2-Bromoethoxy)-2-chlorobenzaldehyde (200 mg, 0.76 mmol) was added to tert-butyl 2,6-diazaspiro[3.3]heptane-2-carboxylate (451 mg, 2.28 mmol), potassium carbonate (210 mg, 1.52 mmol) and sodium iodide (11.4 mg, 0.08 mmol) in DMF (5 mL) at RT. The resulting mixture was stirred at 80° C. for 2 hours. The reaction mixture was diluted with EtOAc (100 mL) and washed with saturated brine (3×15 mL). The organic layer was dried over Na₂SO₄, filtered and evaporated to afford the crude product. The crude product was purified by flash silica chromatography, elution gradient 0 to 100% EtOAc in petroleum ether. Pure fractions were evaporated to dryness to afford tert-butyl 6-(2-(3-chloro-4-formylphenoxy)ethyl)-2,6-diazaspiro[3.3]heptane-2-carboxylate (150 mg, 52%) as a brown solid. ¹H NMR (400 MHz, DMSO-d₆): 1.36 (9H, s), 2.7-2.73 (2H, m), 3.31-3.32 (4H, m), 3.81-3.92 (4H, m), 4.03-4.11 (2H, m), 7.03-7.08 (1H, m), 7.13-7.18 (1H, m), 7.79-7.84 (1H, m), 10.19 (1H, s). m/z: ES+[M+H]+ 381.

### Tert-butyl 6-(2-(4-(9-benzyl-6-(1-methylcyclopropoxy)-9H-purin-8-yl)-3-chlorophenoxy)ethyl)-2,6-diazaspiro[3.3]heptane-2-carboxylate

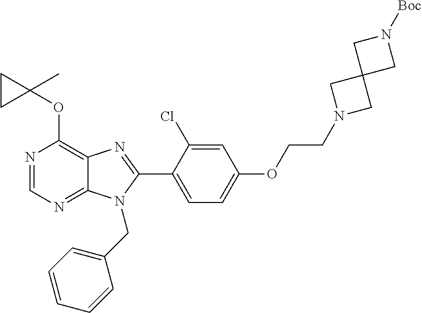 [SMILES: CC(C)(C)OC(=O)N1CC2(CN(CCOc3ccc(-c4nc5c(OC6(C)CC6)ncnc5n4Cc4ccccc4)c(Cl)c3)C2)C1]

Tert-butyl 6-(2-(3-chloro-4-formylphenoxy)ethyl)-2,6-diazaspiro[3.3]heptane-2-carboxylate (140 mg, 0.37 mmol) was added to N4-benzyl-6-(1-methylcyclopropoxy)pyrimidine-4,5-diamine (119 mg, 0.44 mmol) and iron(III) chloride (89 mg, 0.55 mmol) in IPA (5 mL) at rt. The resulting mixture was stirred at 55° C. for 2 hours. The reaction mixture was basified with aqueous 2M NaOH, diluted with EtOAc (100 mL) and washed sequentially with saturated brine (3×20 mL). The organic layer was dried over Na₂SO₄, filtered and evaporated to afford the crude product. The crude product was purified by flash silica chromatography, elution gradient 0 to 20% DCM in MeOH. Pure fractions were evaporated to dryness to afford tert-butyl 6-(2-(4-(9-benzyl-6-(1-methylcyclopropoxy)-9H-purin-8-yl)-3-chlorophenoxy)ethyl)-2,6-diazaspiro[3.3]heptane-2-carboxylate (190 mg, 82%) as a brown solid. ¹H NMR (400 MHz, DMSO-d₆): 0.85 (2H, t), 1.01 (2H, t), 1.36 (9H, s), 1.73 (3H, s), 2.71 (2H, t), 3.30-3.35 (4H, m), 3.81-3.91 (4H, m), 4.00-4.04 (2H, m), 5.21-5.31 (2H, m), 6.87-6.93 (2H, m), 7.00-7.08 (1H, m), 7.18-7.22 (4H, m), 7.40 (1H, d), 8.61 (1H, s). m/z: ES+[M+H]+ 631.

### Synthetic Example 19

### 1-(5-(9-Benzyl-6-(1-methylcyclopropoxy)-9H-purin-8-yl)-4-methylpyridin-2-yl)azetidin-3-amine

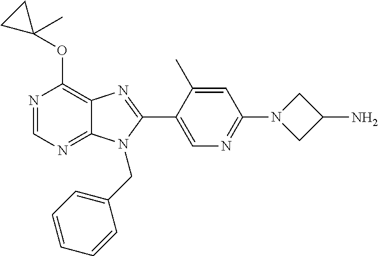 [SMILES: Cc1cc(N2CC(N)C2)ncc1-c1nc2c(OC3(C)CC3)ncnc2n1Cc1ccccc1]

HCl in dioxane (5 mL, 20.00 mmol) was added to tert-butyl (1-(5-(9-benzyl-6-(1-methylcyclopropoxy)-9H-purin-8-yl)-4-methylpyridin-2-yl)azetidin-3-yl)carbamate (200 mg, 0.37 mmol) in EtOAc (1 mL). The resulting mixture was stirred at rt for 2 hours. The solvent was removed under reduced pressure. The crude product was purified by preparative HPLC (XBridge Prep OBD C18 column, 30*150 mm, 5 μm), using decreasingly polar mixtures of water (containing 10 mmol/L NH₄HCO₃ and 0.1% aqueous ammonia) and MeCN as eluents.

Fractions containing the desired compound were evaporated to dryness to afford 1-(5-(9-benzyl-6-(1-methylcyclopropoxy)-9H-purin-8-yl)-4-methylpyridin-2-yl)azetidin-3-amine (51 mg, 31%) as a light yellow solid. ¹H NMR (400 MHz, DMSO-d₆): 0.80-0.92 (2H, m), 0.94-1.06 (2H, m), 1.73 (3H, s), 1.94 (3H, s), 3.59 (2H, dd), 3.83 (1H, tt), 4.15 (2H, dd), 5.31 (2H, s), 6.28 (1H, s), 6.85-6.94 (2H, m), 7.17-7.28 (3H, m), 7.97 (1H, s), 8.58 (1H, s). 2 H not observed. m/z: ES+[M+H]+ 442.

Tert-butyl (1-(5-(9-benzyl-6-(1-methylcyclopropoxy)-9H-purin-8-yl)-4-methylpyridin-2-yl)azetidin-3-yl)carbamate used as a starting material was made as follows:

### 9-Benzyl-8-(6-fluoro-4-methylpyridin-3-yl)-6-(1-methylcyclopropoxy)-9H-purine

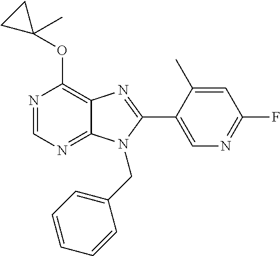 [SMILES: Cc1cc(F)ncc1-c1nc2c(OC3(C)CC3)ncnc2n1Cc1ccccc1]

6-Fluoro-4-methylnicotinaldehyde (1.54 g, 11.10 mmol) was added to N4-benzyl-6-(1-methylcyclopropoxy)pyrimidine-4,5-diamine (2.00 g, 7.40 mmol) in DMSO (30 mL). The resulting mixture was stirred at 100° C. for 16 hours. The reaction mixture was diluted with brine (200 mL) and extracted with EtOAc (4×150 mL). The organic layer was washed with brine (4×150 mL), dried over Na₂SO₄, filtered and evaporated to afford crude product. The crude product was purified by flash silica chromatography, elution gradient 0 to 40% EtOAc in pentane. Pure fractions were evaporated to dryness to afford 9-benzyl-8-(6-fluoro-4-methylpyridin-3-yl)-6-(1-methylcyclopropoxy)-9H-purine (1.90 g, 66%) as a yellow solid. ¹H NMR (400 MHz, DMSO-d6): 0.84-0.88 (2H, m), 1.02-1.05 (2H, m), 1.75 (3H, s), 1.99 (3H, d), 5.35 (2H, s), 6.89 (2H, t), 7.22 (4H, d), 8.25 (1H, s), 8.68 (1H, s). m/z: ES+[M+H]+ 390.

### Tert-butyl (1-(5-(9-benzyl-6-(1-methylcyclopropoxy)-9H-purin-8-yl)-4-methylpyridin-2-yl)azetidin-3-yl)carbamate

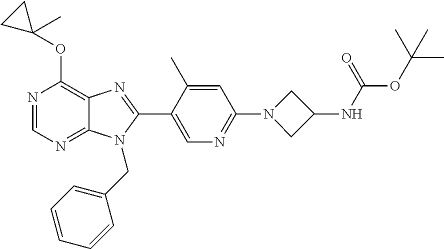 [SMILES: Cc1cc(N2CC(NC(=O)OC(C)(C)C)C2)ncc1-c1nc2c(OC3(C)CC3)ncnc2n1Cc1ccccc1]

Tert-butyl azetidin-3-ylcarbamate, HCl (193 mg, 0.92 mmol) was added to 9-benzyl-8-(6-fluoro-4-methylpyridin-3-yl)-6-(1-methylcyclopropoxy)-9H-purine (180 mg, 0.46 mmol) and DIEA (0.484 ml, 2.77 mmol) in DMSO (1 mL). The resulting mixture was stirred at 100° C. for 1 hour. The reaction mixture was quenched with saturated aq. NH₄Cl (50 mL) and extracted with EtOAc (40 mL×3). The organic layer was washed with brine (3×50 mL), dried over Na₂SO₄, filtered and evaporated to afford tert-butyl (1-(5-(9-benzyl-6-(1-methylcyclopropoxy)-9H-purin-8-yl)-4-methylpyridin-2-yl)azetidin-3-yl)carbamate (0.25 g) used in the next step directly without further purification. m/z: ES+[M+H]+ 542.

### Synthetic Example 20

### 9-Benzyl-8-(4-chloro-6-(2-(4-methylpiperazin-1-yl)ethoxy)pyridin-3-yl)-6-(1-methylcyclopropoxy)-9H-purine

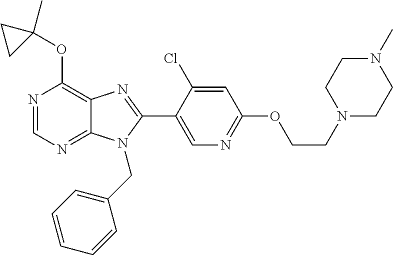 [SMILES: CN1CCN(CCOc2cc(Cl)c(-c3nc4c(OC5(C)CC5)ncnc4n3Cc3ccccc3)cn2)CC1]

RockPhos Pd G3 (18 mg, 0.02 mmol) was added to 9-benzyl-8-(6-bromo-4-chloropyridin-3-yl)-6-(1-methylcyclopropoxy)-9H-purine (100 mg, 0.21 mmol), 2-(4-methylpiperazin-1-yl)ethan-1-ol (30.6 mg, 0.21 mmol) and Cₛ₂CO₃ (208 mg, 0.64 mmol) in toluene (2 mL). The resulting mixture was stirred at 100° C. for 1 hour. The solvent was removed under reduced pressure. The crude product was purified by flash C18-flash chromatography, elution gradient 5 to 100% MeCN in water (0.01% NH₄HCO₃). Pure fractions were evaporated to dryness to afford 9-benzyl-8-(4-chloro-6-(2-(4-methylpiperazin-1-yl)ethoxy)pyridin-3-yl)-6-(1-methylcyclopropoxy)-9H-purine (16 mg, 14%) as a white solid. ¹H NMR (400 MHz, DMSO-d6): 0.86 (2H, m), 1.03 (2H, m), 1.74 (3H, s), 2.16 (3H, s), 2.25-2.42 (4H, m), 2.52 (4H, m), 2.70 (2H, t), 4.45 (2H, t), 5.35 (2H, s), 6.93 (2H, m), 7.17-7.27 (4H, m), 8.28 (1H, s), 8.65 (1H, s). m/z: ES+[M+H]+ 534.

9-Benzyl-8-(6-bromo-4-chloropyridin-3-yl)-6-(1-methylcyclopropoxy)-9H-purine used as a starting material was made as follows:

### 9-Benzyl-8-(6-bromo-4-chloropyridin-3-yl)-6-(1-methylcyclopropoxy)-9H-purine

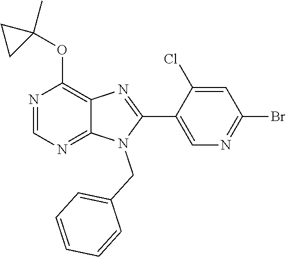 [SMILES: CC1(Oc2ncnc3c2nc(-c2cnc(Br)cc2Cl)n3Cc2ccccc2)CC1]

N4-Benzyl-6-(1-methylcyclopropoxy)pyrimidine-4,5-diamine (180 mg, 0.67 mmol, Synthetic Example 4 Intermediate) was added to 6-bromo-4-chloronicotinaldehyde (220 mg, 1.00 mmol) in DMSO (2 mL). The resulting mixture was stirred at 80° C. for 1 hour. AcOH (0.38 mL, 6.66 mmol) was added to the mixture and the resulting mixture was stirred at 80° C. for 1 day. The reaction mixture was evaporated to afford crude product. The residue was purified by flash C18-flash chromatography, elution gradient 0 to 100% MeCN in water (containing 0.1% NH₄HCO₃). Pure fractions were evaporated to dryness to afford 9-benzyl-8-(6-bromo-4-chloropyridin-3-yl)-6-(1-methylcyclopropoxy)-9H-purine (120 mg, 38%) as a brown solid. ¹H NMR (400 MHz, DMSO-d₆): 0.86 (2H, t), 1.02 (2H, t), 1.73 (3H, s), 5.38 (2H, s), 6.89-6.99 (2H, m), 7.15-7.30 (3H, m), 8.16 (1H, s), 8.53 (1H, s), 8.68 (1H, s). m/z: ES+[M+H]+ 470.

### Synthetic Example 21

### 9-Benzyl-8-(4-methyl-6-(2-(pyrrolidin-1-yl)ethoxy)pyridin-3-yl)-6-(1-methylcyclopropoxy)-9H-purine

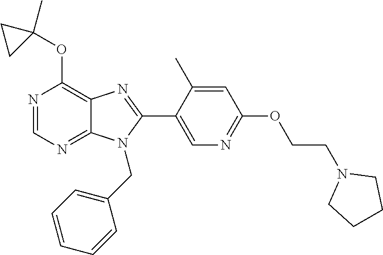 [SMILES: Cc1cc(OCCN2CCCC2)ncc1-c1nc2c(OC3(C)CC3)ncnc2n1Cc1ccccc1]

NaH (23 mg, 0.58 mmol) was added to 2-(pyrrolidin-1-yl)ethan-1-ol (89 mg, 0.77 mmol) in DMF (1 mL). The resulting mixture was stirred at rt for 5 minutes. Then 9-benzyl-8-(6-fluoro-4-methylpyridin-3-yl)-6-(1-methylcyclopropoxy)-9H-purine (150 mg, 0.39 mmol, Synthetic Example 19 Intermediate) was added to the mixture. The resulting mixture was stirred at rt for 1 hour. The crude product was purified by preparative HPLC (XBridge Shield RP18 OBD column, 30*150 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness to afford 9-benzyl-8-(4-methyl-6-(2-(pyrrolidin-1-yl)ethoxy)pyridin-3-yl)-6-(1-methylcyclopropoxy)-9H-purine (47 mg, 25%) as a light yellow solid. ¹H NMR (400 MHz, DMSO-d₆): 0.81-0.88 (2H, m), 1.01-1.03 (2H, m), 1.68 (4H, p), 1.73 (3H, s), 1.92 (3H, s), 2.78 (2H, t), 4.40 (2H, t), 5.33 (2H, s), 6.79 (1H, s), 6.84-6.91 (2H, m), 7.18-7.25 (3H, m), 8.12 (1H, s), 8.63 (1H, s). 4 H not observed. m/z: ES+[M+H]+ 485.

### Synthetic Example 22

### 9-Benzyl-8-(4-methyl-5-(2-(piperazin-1-yl)ethoxy)pyridin-3-yl)-6-(1-methylcyclopropoxy)-9H-purine

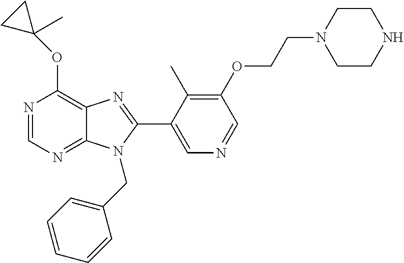 [SMILES: Cc1c(OCCN2CCNCC2)cncc1-c1nc2c(OC3(C)CC3)ncnc2n1Cc1ccccc1]

HCl in dioxane (3 mL, 3.00 mmol) was added to tert-butyl 4-(2-((5-(9-benzyl-6-(1-methylcyclopropoxy)-9H-purin-8-yl)-4-methylpyridin-3-yl)oxy)ethyl)piperazine-1-carboxylate (150 mg, 0.25 mmol) in EtOAc (2 mL). The resulting mixture was stirred at rt for 1 hour. The solvent was removed under reduced pressure. The crude product was purified by preparative HPLC (XBridge Prep OBD C18 column, 30*150 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness to afford 9-benzyl-8-(4-methyl-5-(2-(piperazin-1-yl)ethoxy)pyridin-3-yl)-6-(1-methylcyclopropoxy)-9H-purine (73 mg, 58%) as a brown solid. ¹H NMR (400 MHz, DMSO-d₆): 0.82-0.88 (2H, m), 0.99-1.08 (2H, m), 1.74 (3H, s), 1.78 (3H, s), 2.30-2.45 (4H, m), 2.60-2.77 (6H, m), 4.26 (2H, t), 5.30 (2H, s), 6.74-6.94 (2H, m), 7.17-7.21 (3H, m), 8.15 (1H, s), 8.44 (1H, s), 8.65 (1H, s). 1 H not observed. m/z: ES+[M+H]+ 500.

Tert-butyl 4-(2-((5-(9-benzyl-6-(1-methylcyclopropoxy)-9H-purin-8-yl)-4-methylpyridin-3-yl)oxy)ethyl)piperazine-1-carboxylate used as a starting material was made as follows:

### Tert-butyl 4-(2-((5-bromo-4-methylpyridin-3-yl)oxy)ethyl)piperazine-1-carboxylate

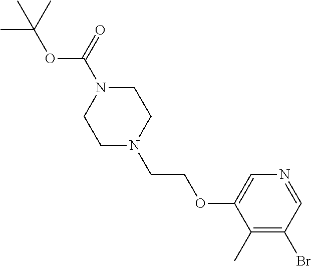 [SMILES: Cc1c(Br)cncc1OCCN1CCN(C(=O)OC(C)(C)C)CC1]

NaH (0.631 g, 15.79 mmol) was added to tert-butyl 4-(2-hydroxyethyl)piperazine-1-carboxylate (5.45 g, 23.68 mmol) in DMF (5 mL) at 0° C. The resulting mixture was stirred at rt for 20 minutes before the addition of 3-bromo-5-fluoro-4-methylpyridine (1.5 g, 7.89 mmol). The resulting mixture was stirred at rt for 1 hour. The reaction mixture was quenched with saturated aq. NH₄Cl (50 mL) and extracted with EtOAc (3×30 mL). The combined organics were dried over Na₂SO₄, filtered and evaporated to afford crude product. The crude product was purified by flash C18-flash chromatography, elution gradient 5 to 80% MeCN in water (0.1% NH₄HCO₃). Pure fractions were evaporated to dryness to afford tert-butyl 4-(2-((5-bromo-4-methylpyridin-3-yl)oxy)ethyl)piperazine-1-carboxylate (0.840 g, 27%) as a yellow solid. ¹H NMR (400 MHz, DMSO-d₆): 1.39 (9H, s), 2.26 (3H, s), 2.43-2.46 (4H, m), 2.76 (2H, t), 3.28-3.32 (4H, m), 4.24 (2H, t), 8.28 (1H, s), 8.31 (1H, s). m/z: ES+[M+H]+ 400.

### Tert-butyl 4-(2-((5-(methoxycarbonyl)-4-methylpyridin-3-yl)oxy)ethyl)piperazine-1-carboxylate

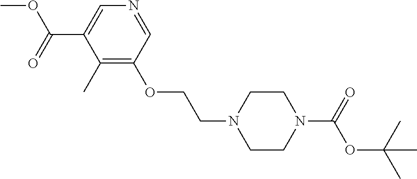 [SMILES: COC(=O)c1cncc(OCCN2CCN(C(=O)OC(C)(C)C)CC2)c1C]

Tert-butyl 4-(2-((5-bromo-4-methylpyridin-3-yl)oxy)ethyl)piperazine-1-carboxylate (80 mg, 2.00 mmol), PdCl₂(dppf) (146 mg, 0.20 mmol) and TEA (0.836 ml, 6.00 mmol) in MeOH (20 mL) were stirred under an atmosphere of carbon monoxide at 10 atm and 100° C. for 16 hours. The solvent was removed under reduced pressure. The crude product was purified by flash C18-flash chromatography, elution gradient 5 to 80% MeCN in water (0.1% NH₄HCO₃). Pure fractions were evaporated to dryness to afford tert-butyl 4-(2-((5-(methoxycarbonyl)-4-methylpyridin-3-yl)oxy)ethyl)piperazine-1-carboxylate (700 mg, 92%) as a yellow solid. ¹H NMR (300 MHz, DMSO-d₆): 1.37 (9H, s), 2.36 (3H, s), 2.41-2.45 (4H, m), 2.75 (2H, t), 3.26-3.30 (4H, m), 3.84 (3H, s), 4.24 (2H, t), 8.44 (1H, s), 8.51 (1H, s). m/z: ES+[M+H]+ 380.

### Tert-butyl 4-(2-((5-(hydroxymethyl)-4-methylpyridin-3-yl)oxy)ethyl)piperazine-1-carboxylate

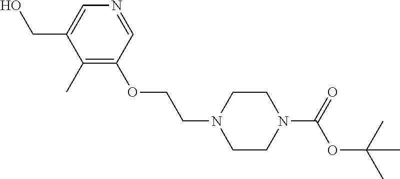 [SMILES: Cc1c(CO)cncc1OCCN1CCN(C(=O)OC(C)(C)C)CC1]

LiAlH₄ (1M in THF, 900 μL, 0.90 mmol) was added to tert-butyl 4-(2-((5-(methoxycarbonyl)-4-methylpyridin-3-yl)oxy)ethyl)piperazine-1-carboxylate (680 mg, 1.79 mmol) in THF (15 mL) at 0° C. The resulting mixture was stirred at 0° C. for 1 hour. The reaction mixture was poured into a 15% aqueous solution of NaOH. The organic layer was separated, dried over Na₂SO₄, filtered and evaporated to afford tert-butyl 4-(2-((5-(hydroxymethyl)-4-methylpyridin-3-yl)oxy)ethyl)piperazine-1-carboxylate (600 mg, 95%) as a yellow gum. The product was used in the next step directly without further purification. ¹H NMR (400 MHz, DMSO-d₆): 1.40 (9H, s), 2.14 (3H, s), 2.44-2.47 (4H, m), 2.76 (2H, t), 3.30-3.32 (4H, m), 4.19 (2H, t), 4.51 (2H, d), 5.15 (1H, t), 8.14 (1H, s), 8.19 (1H, s). m/z: ES+[M+H]+ 352.

### Tert-butyl 4-(2-((5-formyl-4-methylpyridin-3-yl)oxy)ethyl)piperazine-1-carboxylate

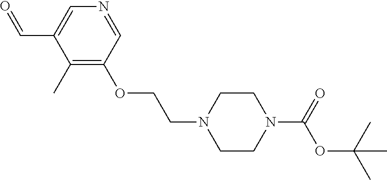 [SMILES: Cc1c(C=O)cncc1OCCN1CCN(C(=O)OC(C)(C)C)CC1]

Manganese(IV) oxide (866 mg, 5.78 mmol) was added to tert-butyl 4-(2-((5-(hydroxymethyl)-4-methylpyridin-3-yl)oxy)ethyl)piperazine-1-carboxylate (580 mg, 1.65 mmol) in EtOAc (10 mL). The resulting mixture was stirred at 80° C. for 16 hours. The mixture was filtered through a Celite pad. The crude product was purified by flash silica chromatography, elution gradient 0 to 10% MeOH in DCM. Pure fractions were evaporated to dryness to afford tert-butyl 4-(2-((5-formyl-4-methylpyridin-3-yl)oxy)ethyl)piperazine-1-carboxylate (0.420 g, 73%) as a yellow solid. ¹H NMR (300 MHz, DMSO-d₆): 1.37 (9H, s), 2.42-2.49 (7H, m), 2.76 (2H, t), 3.22-3.30 (4H, m), 4.26 (2H, t), 8.51 (1H, s), 8.57 (1H, s), 10.28 (1H, s). m/z: ES+[M+H]+ 350.

### Tert-butyl 4-(2-((5-(9-benzyl-6-(1-methylcyclopropoxy)-9H-purin-8-yl)-4-methylpyridin-3-yl)oxy)ethyl)piperazine-1-carboxylate

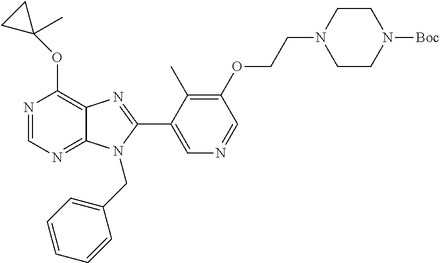 [SMILES: Cc1c(OCCN2CCN(C(=O)OC(C)(C)C)CC2)cncc1-c1nc2c(OC3(C)CC3)ncnc2n1Cc1ccccc1]

N4-Benzyl-6-(1-methylcyclopropoxy)pyrimidine-4,5-diamine (150 mg, 0.55 mmol, Synthetic Example 4 Intermediate) and tert-butyl 4-(2-((5-formyl-4-methylpyridin-3-yl)oxy)ethyl)piperazine-1-carboxylate (291 mg, 0.83 mmol) in DMSO (3 mL) were stirred at 100° C. for 24 hours. The crude product was purified by flash C18-flash chromatography, elution gradient 5 to 80% MeCN in water (containing 0.1% NH₄HCO₃). Pure fractions were evaporated to dryness to afford tert-butyl 4-(2-((5-(9-benzyl-6-(1-methylcyclopropoxy)-9H-purin-8-yl)-4-methylpyridin-3-yl)oxy)ethyl)piperazine-1-carboxylate (170 mg, 51%) as a yellow solid. ¹H NMR (300 MHz, DMSO-d₆): 0.81-0.85 (2H, m), 0.99-1.06 (2H, m), 1.38 (9H, s), 1.72 (3H, s), 1.76 (3H, s), 2.41-2.45 (4H, m), 2.75 (2H, t), 3.27-3.31 (4H, m), 4.26 (2H, t), 5.28 (2H, s), 6.74-6.89 (2H, m), 7.10-7.27 (3H, m), 8.14 (1H, s), 8.42 (1H, s), 8.64 (1H, s). m/z: ES+[M+H]+ 600.

### Synthetic Example 23

### 8-(2-chloro-4-(2-(piperazin-1-yl)ethoxy)phenyl)-9-((6-chloropyridin-2-yl)methyl)-6-(1-methylcyclopropoxy)-9H-purine

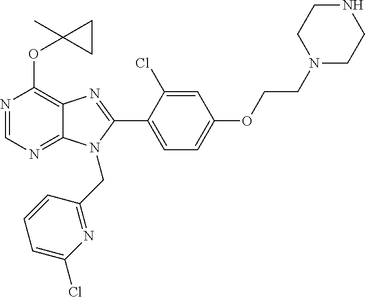 [SMILES: CC1(Oc2ncnc3c2nc(-c2ccc(OCCN4CCNCC4)cc2Cl)n3Cc2cccc(Cl)n2)CC1]

Tert-butyl 4-(2-(3-chloro-4-(6-(1-methylcyclopropoxy)-9H-purin-8-yl)phenoxy)ethyl)piperazine-1-carboxylate (200 mg, 0.38 mmol) was added into DMF (1 mL), followed by Cₛ₂CO₃ (246 mg, 0.76 mmol) and 2-chloro-6-(chloromethyl)pyridine (184 mg, 1.13 mmol). The resulting mixture was stirred at 100° C. for 2 hours.

The reaction mixture was poured into aq. saturated NaHCO₃ (100 mL), extracted with ethyl acetate (100 mL×3). The combined organic layers were washed with brine (100 mL), dried over Na₂SO₄, filtered and concentrated under reduced pressure to give a residue. To the above residue was added TFA (1 mL) at 25° C., and the mixture was stirred for 0.5 h. The solvent was removed under reduced pressure. The crude product was purified by flash C18-flash chromatography, elution gradient 0 to 100% MeCN in water. Fractions were evaporated to dryness to afford a mixture containing 8-(2-chloro-4-(2-(piperazin-1-yl)ethoxy)phenyl)-9-((6-chloropyridin-2-yl)methyl)-6-(1-methylcyclopropoxy)-9H-purine and 8-(2-chloro-4-(2-(piperazin-1-yl)ethoxy)phenyl)-7-((6-chloropyridin-2-yl)methyl)-6-(1-methylcyclopropoxy)-7H-purine as a pale yellow solid. The mixture was purified by preparative HPLC (YMC-Actus Triart C18 ExRS column, 30*150 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness to afford 8-(2-chloro-4-(2-(piperazin-1-yl)ethoxy)phenyl)-9-((6-chloropyridin-2-yl)methyl)-6-(1-methylcyclopropoxy)-9H-purine as a yellow oil (9 mg).

¹H NMR (400 MHz, DMSO-d₆): 0.85 (2H, t), 1.01 (2H, t), 1.73 (3H, s), 2.42-2.51 (4H, m), 2.65-2.72 (6H, m), 4.14 (2H, t), 5.37 (2H, s), 7.00 (1H, dd), 7.08 (1H, d), 7.19 (1H, d), 7.34 (1H, d), 7.42 (1H, d), 7.71 (1H, t), 8.57 (1H, s). One proton not observed. m/z: ES+[M+H]+ 554.

Tert-butyl 4-(2-(3-chloro-4-(6-(1-methylcyclopropoxy)-9H-purin-8-yl)phenoxy)ethyl)piperazine-1-carboxylate used as starting material was made as follows:

### 6-(1-methylcyclopropoxy)-9-(tetrahydro-2H-pyran-2-yl)-9H-purine

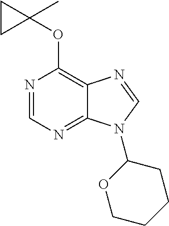 [SMILES: CC1(Oc2ncnc3c2ncn3C2CCCCO2)CC1]

A solution of sodium hydride (60% in mineral oil, 4.1 g, 102 mmol) in dry THF (60 mL) was cooled over ice, then a solution of 1-methylcyclopropan-1-ol (7.21 g, 100 mmol) in THF (20 mL) was added slowly. The reaction mixture stirred for 15 mins, under N2, then a solution of 6-chloro-9-(tetrahydro-2H-pyran-2-yl)-9H-purine (11.94 g, 50.0 mmol) in THF (20 mL) added slowly. The reaction warmed to RT and stirred for 2 hrs. The reaction mixture was then heated at 60° C. for 2 hrs. The reaction was quenched with the addition of ice-water/EtOAc and the aqueous phase was extracted (EtOAc x 2, 100 mL). The combined organics were passed through a hydrophobic frit and concentrated in vacuo. Purification on silica gel (330 g, 0-50% EtOAc in heptanes) yielded 6-(1-methylcyclopropoxy)-9-(tetrahydro-2H-pyran-2-yl)-9H-purine (7.65 g, 56%) as a yellow gum. ¹H NMR (500 MHz, CDCl₃): 0.79-0.84 (2H, m), 1.1-1.16 (2H, m), 1.62-1.69 (1H, m), 1.74-1.81 (5H, m), 2.04-2.14 (3H, m), 3.78 (1H, td), 4.13-4.21 (1H, m), 5.75 (1H, dd), 8.12 (1H, s), 8.61 (1H, s). m/z: ES+[M+H]+ 275.

### 8-bromo-6-(1-methylcyclopropoxy)-9-(tetrahydro-2H-pyran-2-yl)-9H-purine

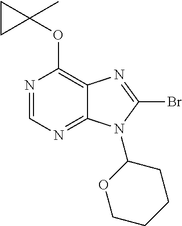 [SMILES: CC1(Oc2ncnc3c2nc(Br)n3C2CCCCO2)CC1]

To a solution of diisopropylamine (5.9 ml, 41.98 mmol) in dry THF (60 ml) under N2 and cooled over dry ice/acetone bath was added butyllithium (2.5M in hexanes) (15.6 mL, 39.0 mmol) slowly. The reaction mixture stirred for 5 mins, then a solution of 6-(1-methylcyclopropoxy)-9-(tetrahydro-2H-pyran-2-yl)-9H-purine (7.65 g, 27.9 mmol) in THF (30 mL) was added slowly. The reaction stirred for 15 mins. Finally, a solution of 1,2-dibromo-1,1,2,2-tetrachloroethane (18.14 g, 55.7 mmol) in THF (30 mL) was added and the reaction mixture was stirred for 3 hrs. The reaction was then quenched with the addition of 5 mL sat. aq. NH₄Cl and allowed to warm to RT slowly. The reaction mixture was concentrated in vacuo and re-dissolved in EtOAc (150 mL) and water (150 mL). The layers were separated and the aqueous layer was further extracted with EtOAc (150 mL×2). The organics were combined via hydrophobic frit and dried in vacuo. The crude product 8-bromo-6-(1-methylcyclopropoxy)-9-(tetrahydro-2H-pyran-2-yl)-9H-purine was used directly in the next step.

### 8-bromo-6-(1-methylcyclopropoxy)-9H-purine

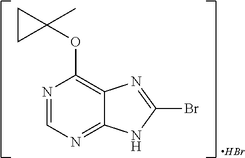

To 8-bromo-6-(1-methylcyclopropoxy)-9-(tetrahydro-2H-pyran-2-yl)-9H-purine (9.54 g, 27 mmol) was added 1,4-dioxane (40 mL) and hydrogen bromide (33% in acetic acid, 15 mL, 82.8 mmol) slowly over ice. The reaction stirred at RT for 5 mins and then the reaction mixture was then diluted with TBME and filtered. The solids washed with TBME and dried under vacuum over the weekend. 8-Bromo-6-(1-methylcyclopropoxy)-9H-purine-HBr salt (9.18 g, 97%) was isolated as a beige solid. ¹H NMR (500 MHz, CD₃OD): 0.91-0.97 (2H, m), 1.16-1.21 (2H, m), 1.81 (3H, s), 8.91 (1H, s). One proton not observed. m/z: ES+[M+H]+ 271 (⁸¹Br isotope).

### Tert-butyl 4-(2-(3-chloro-4-(6-(1-methylcyclopropoxy)-9H-purin-8-yl)phenoxy)ethyl)piperazine-1-carboxylate

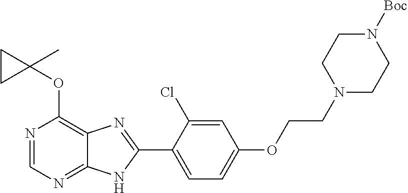 [SMILES: CC(C)(C)OC(=O)N1CCN(CCOc2ccc(-c3nc4c(OC5(C)CC5)ncnc4[nH]3)c(Cl)c2)CC1]

A degassed mixture of dioxane (42 mL) and water (8.4 mL) was added to a multineck flask containing 8-bromo-6-(1-methylcyclopropoxy)-9H-purine-HBr salt (3.50 g, 10.0 mmol), tert-butyl 4-(2-(3-chloro-4-(4,4,5,5-tetramethyl-1,3,2-dioxaborolan-2-yl)phenoxy)ethyl)piperazine-1-carboxylate (6.09 g, 13.05 mmol, Synthetic Example 5 intermediate), tetrakis(triphenylphosphine)palladium(0) (0.59 g, 0.51 mmol) and cesium carbonate (9.78 g, 30.0 mmol), under nitrogen. The reaction stirred at 120° C. for 3 hrs and then increased to 140° C. Further tetrakis(triphenylphosphine)palladium(0) (0.5 g, 0.43 mmol) was added. The mixture was heated at 140° C. overnight and then cooled to RT. Water (150 mL) was then added and the mixture was extracted with EtOAc (3×150 mL). The organics were combined via a hydrophobic frit and concentrated in vacuo. Purification on silica gel (220 g, 0-100% EtOAc in heptanes and then 0-50% EtOAc/EtOH 3:1 in EtOAc) yielded tert-butyl 4-(2-(3-chloro-4-(6-(1-methylcyclopropoxy)-9H-purin-8-yl)phenoxy)ethyl)piperazine-1-carboxylate (3.61 g, 68%) as a light yellow solid. ¹H NMR (500 MHz, DMSO-d₆): 0.84 (2H, s), 1.01 (2H, s), 1.39 (9H, s), 1.72 (3H, s), 2.43-2.46 (4H, m), 2.74 (2H, t), 3.32 (4H, s), 4.21 (2H, t), 7.10 (1H, dd), 7.25 (1H, d), 7.72 (1H, d), 8.52 (1H, s), 13.53 (1H, s). m/z: ES+[M+H]+ 529.

### Synthetic Example 24

### 9-Benzyl-8-(4-methyl-6-(2-(piperazin-1-yl)ethoxy)pyridin-3-yl)-6-(1-methylcyclobutoxy)-9H-purine

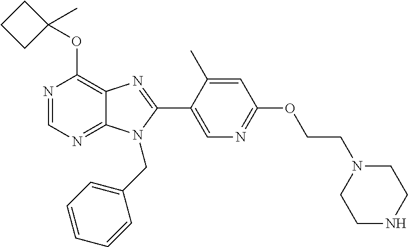 [SMILES: Cc1cc(OCCN2CCNCC2)ncc1-c1nc2c(OC3(C)CCC3)ncnc2n1Cc1ccccc1]

Sodium hydride (36 mg, 1.49 mmol) was added in one portion to 2,2,2-trifluoro-1-(4-(2-hydroxyethyl)piperazin-1-yl)ethan-1-one (81 mg, 0.36 mmol) and 9-benzyl-8-(6-fluoro-4-methylpyridin-3-yl)-6-(1-methylcyclobutoxy)-9H-purine (120 mg, 0.30 mmol) in THF (7 mL) at 0° C. under nitrogen. The resulting solution was stirred at 25° C. for 4 hours. The reaction mixture was quenched with saturated NH₄Cl (2 mL) and the solvent removed under reduced pressure. The crude product was purified by preparative HPLC (Xselect CSH C18 OBD column, 30*150 mm, 5 μm), using decreasingly polar mixtures of water (containing 10 mmol/L NH₄HCO₃ and 0.05% aqueous ammonia) and MeCN as eluents. Fractions containing the desired compound were evaporated to dryness to afford 9-benzyl-8-(4-methyl-6-(2-(piperazin-1-yl)ethoxy)pyridin-3-yl)-6-(1-methylcyclobutoxy)-9H-purine (65 mg, 43%) as a white solid. ¹H NMR (300 MHz, DMSO-d₆): 1.63-1.88 (5H, m), 1.92 (3H, s), 2.25-2.49 (8H, m), 2.6-2.8 (6H, m), 4.40 (2H, t), 5.31 (2H, s), 6.79 (1H, s), 6.83-7 (2H, m), 7.11-7.33 (3H, m), 8.12 (1H, s), 8.52 (1H, s). 1H not observed. m/z: ES+[M+H]+ 514.

9-Benzyl-8-(6-fluoro-4-methylpyridin-3-yl)-6-(1-methylcyclobutoxy)-9H-purine used as a starting material was made as follows.

### 9-Benzyl-8-(6-fluoro-4-methylpyridin-3-yl)-6-(1-methylcyclobutoxy)-9H-purine

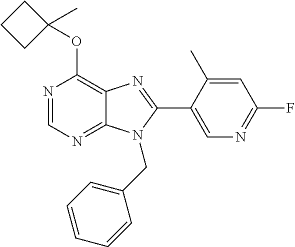 [SMILES: Cc1cc(F)ncc1-c1nc2c(OC3(C)CCC3)ncnc2n1Cc1ccccc1]

Pd(Ph₃P)₄ (46 mg, 0.04 mmol) was added in one portion to 9-benzyl-8-bromo-6-(1-methylcyclobutoxy)-9H-purine (150 mg, 0.40 mmol, Synthetic Example 12 intermediate), (6-fluoro-4-methylpyridin-3-yl)boronic acid (93 mg, 0.60 mmol) and cesium carbonate (262 mg, 0.80 mmol) in 1,4-dioxane (20 mL) and water (5 mL) at 25° C. under nitrogen. The resulting solution was stirred at 100° C. for 4 hours. The reaction mixture was diluted with water. The aqueous layer was extracted with EtOAc (3×20 mL). The organic layer was dried over Na₂SO₄, filtered and evaporated to afford crude product. The crude product was purified by flash silica chromatography, elution gradient 0 to 50% EtOAc in petroleum ether. Pure fractions were evaporated to dryness to afford 9-benzyl-8-(6-fluoro-4-methylpyridin-3-yl)-6-(1-methylcyclobutoxy)-9H-purine (150 mg, 93%) as a colourless solid. ¹H NMR (300 MHz, CDCl₃): 1.67-1.95 (5H, m), 2.00 (3H, s), 2.3-2.48 (2H, m), 2.48-2.73 (2H, m), 5.30 (2H, s), 6.75-7.00 (3H, m), 7.12-7.26 (3H, m), 8.06 (1H, s), 8.59 (1H, s). m/z: ES+[M+H]+ 404.

2,2,2-Trifluoro-1-(4-(2-hydroxyethyl)piperazin-1-yl)ethan-1-one used as a starting material was made as follows:

### 2,2,2-Trifluoro-1-(4-(2-hydroxyethyl)piperazin-1-yl)ethan-1-one

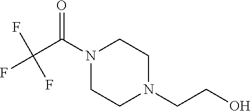 [SMILES: O=C(N1CCN(CCO)CC1)C(F)(F)F]

2,2,2-Trifluoroacetic anhydride (7.74 g, 36.87 mmol) was added dropwise to 2-(piperazin-1-yl)ethan-1-ol (4.00 g, 30.72 mmol) and triethylamine (4.66 g, 46.09 mmol) in DCM (100 mL) at 0° C. under nitrogen. The resulting solution was stirred at 25° C. for 4 hours. The solvent was removed under reduced pressure to get crude product. The crude product was purified by flash silica chromatography, elution gradient 0 to 20% MeOH in DCM. Pure fractions were evaporated to dryness to afford 2,2,2-trifluoro-1-(4-(2-hydroxyethyl)piperazin-1-yl)ethan-1-one (3.80 g, 55%) as a colourless oil. ¹H NMR (300 MHz, CDCl₃): 2.45-2.76 (7H, m), 3.53-3.92 (6H, m). m/z: ES+[M+H]+ 227.

### Synthetic Example 25

### 2-(3-Chloro-4-(6-(1-methylcyclopropoxy)-9-((4-methylpyridin-2-yl)methyl)-9H-purin-8-yl)phenoxy)-N,N-dimethylethan-1-amine

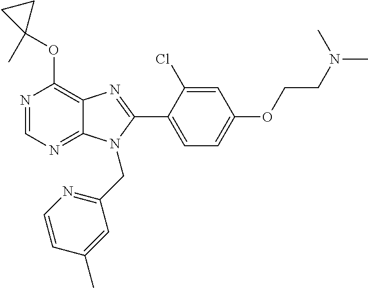 [SMILES: Cc1ccnc(Cn2c(-c3ccc(OCCN(C)C)cc3Cl)nc3c(OC4(C)CC4)ncnc32)c1]

Dimethylamine (0.48 mL, 2.84 mmol) was added to 8-(4-(2-bromoethoxy)-2-chlorophenyl)-6-(1-methylcyclopropoxy)-9-((4-methylpyridin-2-yl)methyl)-9H-purine (150 mg, 0.28 mmol) and potassium carbonate (196 mg, 1.42 mmol) in MeCN (2 mL) under air. The resulting mixture was stirred at 70° C. for 2 hours. The solvent was removed under reduced pressure. The crude product was purified by purified by preparative HPLC (XBridge BEH C18 OBD Prep Column, 5 μm silica, 19 mm diameter, 250 mm length), using decreasingly polar mixtures of water (containing 0.1% aq. NH₃ and 10 mmol/L NH₄HCO₃) and MeCN as eluents. Fractions containing the desired compound were evaporated to dryness to afford 2-(3-chloro-4-(6-(1-methylcyclopropoxy)-9-((4-methylpyridin-2-yl)methyl)-9H-purin-8-yl)phenoxy)-N,N-dimethylethan-1-amine (30 mg, 21%) as a white solid. 1H NMR (400 MHz, DMSO-d₆): 0.86 (2H, t), 1.03 (2H, t), 1.74 (3H, s), 2.19 (3H, s), 2.21 (6H, s), 2.62 (2H, t), 4.12 (2H, t), 5.35 (2H, s), 6.86 (1H, s), 6.96 (1H, dd), 7.03 (1H, d), 7.20 (1H, d), 7.37 (1H, d), 8.18 (1H, d), 8.57 (1H, s). m/z: ES+[M+H]+ 493.

8-(4-(2-Bromoethoxy)-2-chlorophenyl)-6-(1-methylcyclopropoxy)-9-((4-methylpyridin-2-yl)methyl)-9H-purine used as a starting material was made as follows.

### 8-(4-Bromo-2-chlorophenyl)-6-chloro-9H-purine

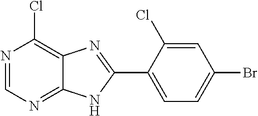 [SMILES: Clc1cc(Br)ccc1-c1nc2c(Cl)ncnc2[nH]1]

Iron(III) chloride (112.0 g, 691.74 mmol) was added to 6-chloropyrimidine-4,5-diamine (20.0 g, 138 mmol) and 4-bromo-2-chlorobenzaldehyde (29.8 g, 136 mmol) in IPA (400 mL) at 25° C. The resulting mixture was stirred at 80° C. for 12 hours. The reaction mixture was concentrated under reduced pressure to remove most of the solvent, then diluted with EtOAc, poured into ice water, and the formed precipitate isolated by filtration. The filter cake was washed with water until the water pH is 7. The solid was dissolved in THF, dried over anhydrous Na₂SO₄, filtered, concentrated to dryness to afford 8-(4-bromo-2-chlorophenyl)-6-chloro-9H-purine (32.0 g, 67%) as a yellow solid. ¹H NMR (300 MHz, DMSO-d₆): 7.68-7.78 (1H, m), 7.86-7.97 (2H, m), 8.60 (1H, s). 1H not observed. m/z: ES+[M+H]+ 343.

### 8-(4-Bromo-2-chlorophenyl)-6-chloro-9-((4-methylpyridin-2-yl)methyl)-9H-purine

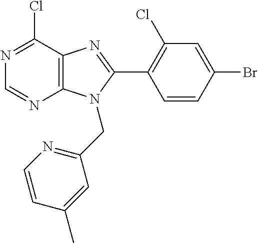 [SMILES: Cc1ccnc(Cn2c(-c3ccc(Br)cc3Cl)nc3c(Cl)ncnc32)c1]

8-(4-Bromo-2-chlorophenyl)-6-chloro-9H-purine (8.00 g, 23.26 mmol) was added to 2-(chloromethyl)-4-methylpyridine (4.94 g, 34.88 mmol) and K₂CO₃ (9.64 g, 69.77 mmol) in MeCN (20 mL). The resulting mixture was stirred at 80° C. for 4 hours. The reaction mixture was diluted with EtOAc (75 mL), and washed sequentially with water (25 mL×3). The organic layer was dried over Na₂SO₄, filtered and evaporated to afford crude product. The crude product was purified by flash silica chromatography, elution gradient 0 to 60% EtOAc in petroleum ether. Pure fractions were evaporated to dryness to afford 8-(4-bromo-2-chlorophenyl)-6-chloro-9-((4-methylpyridin-2-yl)methyl)-9H-purine (4.90 g, 47%) as a white solid. ¹H NMR (300 MHz, DMSO-d₆): 2.21 (3H, s), 5.47 (2H, s), 6.97 (1H, s), 7.06 (1H, d), 7.53 (1H, d), 7.69 (1H, dd), 7.96 (1H, d), 8.16 (1H, d), 8.85 (1H, s). m/z: ES+[M+H]+ 448.

### 8-(4-Bromo-2-chlorophenyl)-6-(1-methylcyclopropoxy)-9-((4-methylpyridin-2-yl)methyl)-9H-purine

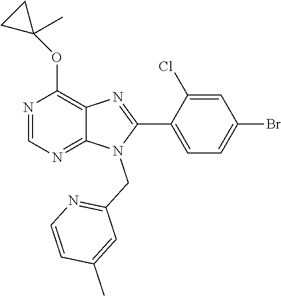 [SMILES: Cc1ccnc(Cn2c(-c3ccc(Br)cc3Cl)nc3c(OC4(C)CC4)ncnc32)c1]

NaH (2.45 g, 61.23 mmol) was added to 8-(4-bromo-2-chlorophenyl)-6-chloro-9-((4-methylpyridin-2-yl)methyl)-9H-purine (5.50 g, 12.2 mmol) and 1-methylcyclopropan-1-ol (2.21 g, 30.6 mmol) in DMF (100 mL) at 0° C. under nitrogen. The resulting mixture was stirred at 25° C. for 2 hours. The reaction mixture was quenched with saturated aq. NH₄Cl (150 mL), extracted with EtOAc (3×100 mL). The organic layer was dried over Na₂SO₄, filtered and evaporated under reduced pressure. The crude product was purified by flash silica chromatography, elution gradient 0 to 50% EtOAc in petroleum ether. Pure fractions were evaporated to dryness to afford 8-(4-bromo-2-chlorophenyl)-6-(1-methylcyclopropoxy)-9-((4-methylpyridin-2-yl)methyl)-9H-purine (4.50 g, 76%) as a pale yellow solid. ¹H NMR (300 MHz, DMSO-d₆): 0.78-0.89 (2H, m), 1.00-1.07 (2H, m), 1.74 (3H, s), 2.20 (3H, s), 5.37 (2H, s), 6.90 (1H, d), 7.04 (1H, dd), 7.45 (1H, d), 7.64 (1H, dd), 7.93 (1H, d), 8.16 (1H, dd), 8.60 (1H, s). m/z: ES+[M+H]+ 484.

### 3-Chloro-4-(6-(1-methylcyclopropoxy)-9-((4-methylpyridin-2-yl)methyl)-9H-purin-8-yl)phenol

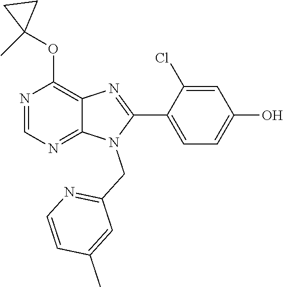 [SMILES: Cc1ccnc(Cn2c(-c3ccc(O)cc3Cl)nc3c(OC4(C)CC4)ncnc32)c1]

1,1′-Bis(diphenylphosphino)ferrocenedichloropalladium (II) dichloromethane adduct (253 mg, 0.31 mmol) was added to 8-(4-bromo-2-chlorophenyl)-6-(1-methylcyclopropoxy)-9-((4-methylpyridin-2-yl)methyl)-9H-purine (1.50 g, 3.09 mmol), 4,4,4′,4′,5,5,5′,5′-octamethyl-2,2′-bi(1,3,2-dioxaborolane) (864 mg, 3.40 mmol) and potassium acetate (607 mg, 6.19 mmol) in 1,4-dioxane (10 mL) at rt under nitrogen. The resulting mixture was stirred at 100° C. for 1 hour. The solvent was removed under reduced pressure. The solid was diluted with THF/water (10 mL). Sodium perborate (1.26 g, 15.47 mmol) was added and the mixture was stirred at RT for a further 30 minutes. The reaction mixture was diluted with EtOAc (150 mL), and washed sequentially with water (50 mL×3). The organic layer was dried over Na₂SO₄, filtered and evaporated to afford crude product. The crude product was purified by flash silica chromatography, elution gradient 0 to 20% MeOH in DCM. Pure fractions were evaporated to dryness to afford 3-chloro-4-(6-(1-methylcyclopropoxy)-9-((4-methylpyridin-2-yl)methyl)-9H-purin-8-yl)phenol (1.00 g, 77%) as a white solid. ¹H NMR (300 MHz, DMSO-d₆): 0.86 (2H, t), 1.02 (2H, t), 1.74 (3H, s), 2.20 (3H, s), 5.34 (2H, s), 6.75 (1H, dd), 6.84 (1H, s), 6.93 (1H, d), 7.04 (1H, d), 7.25 (1H, d), 8.18 (1H, d), 8.56 (1H, s). 1H not observed. m/z: ES+[M+H]+ 422.

### 8-(4-(2-Bromoethoxy)-2-chlorophenyl)-6-(1-methylcyclopropoxy)-9-((4-methylpyridin-2-yl)methyl)-9H-purine

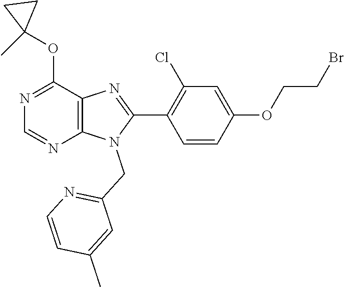 [SMILES: Cc1ccnc(Cn2c(-c3ccc(OCCBr)cc3Cl)nc3c(OC4(C)CC4)ncnc32)c1]

1,2-Dibromoethane (891 mg, 4.74 mmol) was added to K₂CO₃ (197 mg, 1.42 mmol) and 3-chloro-4-(6-(1-methylcyclopropoxy)-9-((4-methylpyridin-2-yl)methyl)-9H-purin-8-yl)phenol (400 mg, 0.95 mmol) in MeCN (20 mL) at rt. The resulting mixture was stirred at 60° C. for 18 hours. The reaction mixture was evaporated to dryness and redissolved in EtOAc (150 mL), and washed sequentially with water (125 mL×3) and saturated brine (100 mL×2). The organic layer was dried over Na₂SO₄, filtered and evaporated to afford crude product. The crude product was purified by flash silica chromatography, elution gradient 40 to 80% EtOAc in petroleum ether. Pure fractions were evaporated to dryness to afford 8-(4-(2-bromoethoxy)-2-chlorophenyl)-6-(1-methylcyclopropoxy)-9-((4-methylpyridin-2-yl)methyl)-9H-purine (230 mg, 46%) as a white solid. ¹H NMR (300 MHz, DMSO-d₆): 0.86 (2H, t), 1.03 (2H, t), 1.74 (3H, s), 2.5-2.52 (3H, m), 3.78-3.87 (2H, m), 4.39-4.45 (2H, m), 5.35 (2H, s), 6.87 (1H, d), 6.97-7.06 (2H, m), 7.23 (1H, d), 7.40 (1H, d), 8.18 (1H, d), 8.57 (1H, s). m/z: ES+[M+H]+ 528.

### Synthetic Example 26

### 1-(2-(3-Chloro-4-(6-(1-methylcyclopropoxy)-9-((4-methylpyridin-2-yl)methyl)-9H-purin-8-yl)phenoxy)ethyl)azetidin-3-ol

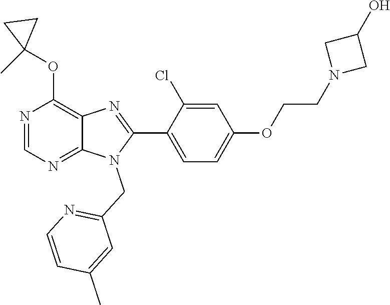 [SMILES: Cc1ccnc(Cn2c(-c3ccc(OCCN4CC(O)C4)cc3Cl)nc3c(OC4(C)CC4)ncnc32)c1]

Azetidin-3-ol (276 mg, 3.78 mmol) was added to 8-(4-(2-bromoethoxy)-2-chlorophenyl)-6-(1-methylcyclopropoxy)-9-((4-methylpyridin-2-yl)methyl)-9H-purine (400 mg, 0.76 mmol, Synthetic Example 25 intermediate) and K₂CO₃ (523 mg, 3.78 mmol) in MeCN (2 mL) under air. The resulting mixture was stirred at 70° C. for 2 hours. The solvent was removed under reduced pressure. The crude product was purified by preparative HPLC (XBridge Prep OBD C18 Column, 5 μm silica, 30 mm diameter, 150 mm length), using decreasingly polar mixtures of water (containing 0.1% aq. NH₃ and 10 mmol/L NH₄HCO₃) and MeCN as eluents. Fractions containing the desired compound were evaporated to dryness to afford 1-(2-(3-chloro-4-(6-(1-methylcyclopropoxy)-9-((4-methylpyridin-2-yl)methyl)-9H-purin-8-yl)phenoxy)ethyl)azetidin-3-ol (24 mg, 6%) as a white solid. ¹H NMR (400 MHz, DMSO-d₆): 0.86 (2H, t), 1.02 (2H, t), 1.74 (3H, s), 2.20 (3H, s), 2.69-2.84 (4H, m), 3.51-3.59 (2H, m), 4.00 (2H, t), 4.12-4.21 (1H, m), 5.27 (1H, d), 5.34 (2H, s), 6.86 (1H, s), 6.93 (1H, dd), 7.03 (1H, d), 7.16 (1H, d), 7.36 (1H, d), 8.18 (1H, d), 8.57 (1H, s). m/z: ES+[M+H]+ 521.

### Synthetic Example 27: Further Examples

The following examples in Table A were synthesised as stated in the notes following Table A.

TABLE A

| Synthetic | | | | LCMS |
|---|---|---|---|---|
| Example | Structure | Name | ¹H NMR | [M + H]+ |
| A1 | 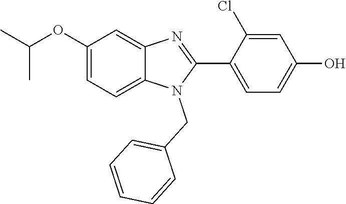 [SMILES: CC(C)Oc1ccc2c(c1)nc(-c1ccc(O)cc1Cl)n2Cc1ccccc1] | 4-(1-benzyl-5- isopropoxy- 1H- benzo[d] imidazol- 2-yl)-3- chlorophenol | (300 MHz, DMSO-d₆): 1.27 (6H, d), 4.60 (1H, p), 5.25 (2H, s), 6.85 (2H, td), 6.93- 7.04 (3H, m), 7.15-7.30 (4H, m), 7.30 (1H, t), 7.37 (1H, d), 10.41 (1H, s) | 393 |
| A2 | 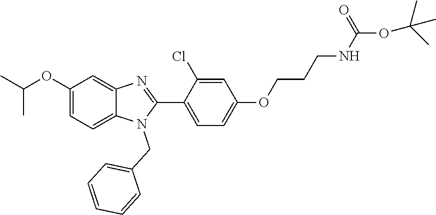 [SMILES: CC(C)Oc1ccc2c(c1)nc(-c1ccc(OCCCNC(=O)OC(C)(C)C)cc1Cl)n2Cc1ccccc1] | tert-butyl (3- (4-(1-benzyl- 5-isopropoxy- 1H- benzo[d] imidazol- 2-yl)-3- chlorophenoxy) propyl) carbamate | (400 MHz, CDCl₃): 1.38 (6H, d), 1.46 (9H, s), 2.03 (2H, q), 3.35 (2H, q), 4.07 (2H, t), 4.58 (1H, hept), 4.74 (1H, s), 5.22 (2H, s), 6.87 (2H, ddd), 6.95-7.02 (2H, m), 7.06 (1H, d), 7.09 (1H, d), 7.25 (3H, dd), 7.35 (1H, d), 7.39 (1H, d) | 550 |
| A3 | 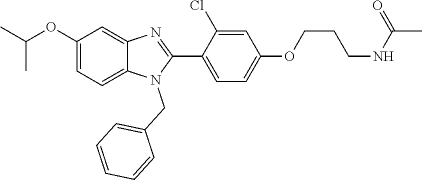 [SMILES: CC(=O)NCCCOc1ccc(-c2nc3cc(OC(C)C)ccc3n2Cc2ccccc2)c(Cl)c1] | N-(3-(4-(1- benzyl-5- isopropoxy- 1H- benzo[d] imidazol- 2-yl)-3- chlorophenoxy) propyl) acetamide | (300 MHz, CDCl₃): 1.36 (6H, d), 1.98 (3H, s), 1.96- 2.09 (2H, m), 3.44 (2H, q), 4.04 (2H, t), 4.56 (1H, p), 5.20 (2H, s), 5.99 (1H, s), 6.85 (2H, ddd), 6.92-7.00 (2H, m), 7.02 (1H, d), 7.07 (1H, d), 7.23 (3H, dd), 7.31 (1H, d), 7.36 (1H, d) | 492 |
| A4 | 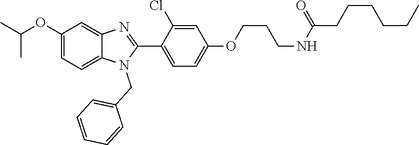 [SMILES: CCCCCCC(=O)NCCCOc1ccc(-c2nc3cc(OC(C)C)ccc3n2Cc2ccccc2)c(Cl)c1] | N-(3-(4-(1- benzyl-5- isopropoxy- 1H- benzo[d] imidazol- 2-yl)-3- chlorophenoxy) propyl) heptanamide | (300 MHz, CDCl₃): 0.81- 0.91 (2H, m), 1.28 (6H, d), 1.35 (6H, d), 1.61 (2H, p), 2.01 (2H, p), 2.17 (2H, t), 3.44 (2H, q), 4.03 (2H, t), 4.55 (1H, p), 5.19 (2H, s), 6.01 (1H, t), 6.84 (2H, ddd), 6.96 (2H, dd), 7.00-7.11 (2H, m), 7.22 (3H, dd), 7.28- 7.40 (2H, m) One proton not observed. | 562 |
| A5 | 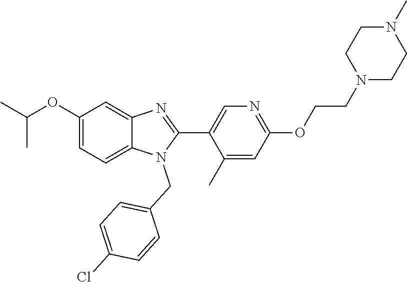 [SMILES: Cc1cc(OCCN2CCN(C)CC2)ncc1-c1nc2cc(OC(C)C)ccc2n1Cc1ccc(Cl)cc1] | 1-(4- chlorobenzyl)- 5- isopropoxy-2- (4-methyl-6- (2-(4- methyl- piperazin-1-yl) ethoxy)pyridin- 3-yl)-1H- benzo[d] imidazole | (400 MHz, DMSO-d₆): 1.28 (6H, d), 2.09 (3H, s), 2.13 (3H, s), 2.30 (4H, s), 2.48 (4H, s),2.68 (2H, t), 4.39 (2H, t), 4.60 (1H, hept), 5.30 (2H, s), 6.81-6.90 (2H, m), 6.93 (2H, d), 7.23 (1H, d), 7.28-7.34 (2H, m), 7.38 (1H, d), 8.12 (1H, s). | 534 |
| A6 | 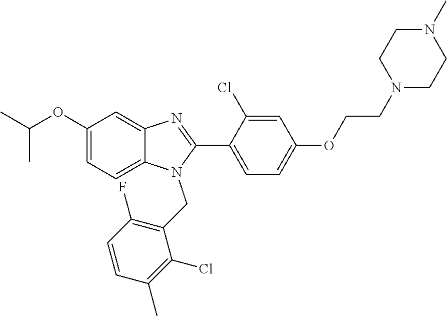 [SMILES: Cc1ccc(F)c(Cn2c(-c3ccc(OCCN4CCN(C)CC4)cc3Cl)nc3cc(OC(C)C)ccc32)c1Cl] | 2-(2-chloro-4- (2-(4- methyl- piperazin-1- yl)ethoxy) phenyl)-1-(2- chloro-6- fluoro-3- methylbenzyl)- 5- isopropoxy- 1H- benzo[d] imidazole | No NMR available | 585 |
| A7 | 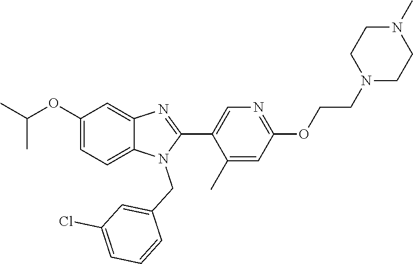 [SMILES: Cc1cc(OCCN2CCN(C)CC2)ncc1-c1nc2cc(OC(C)C)ccc2n1Cc1cccc(Cl)c1] | 1-(3- chlorobenzyl)- 5- isopropoxy-2- (4-methyl-6- (2-(4- methyl- piperazin-1- yl)ethoxy) pyridin- 3-yl)-1H- benzo[d] imidazole | (300 MHz, DMSO-d₆): 1.26 (6H, d), 2.05 (3H, s), 2.10 (3H, s), 2.27 (4H, s), 2.44 (4H, s), 2.65 (2H, t), 4.37 (2H, t), 4.59 (1H, p), 5.30 (2H, s), 6.75-6.89 (3H, m), 6.93 (1H, s), 7.17-7.29 (3H, m), 7.42 (1H, d), 8.10 (1H, s). | 534 |
| A8 | 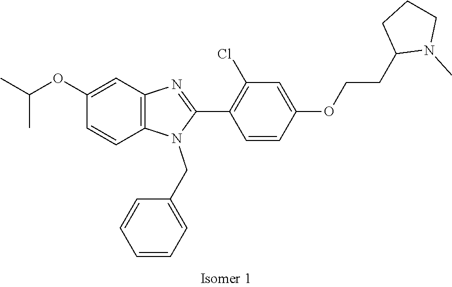 [SMILES: CC(C)Oc1ccc2c(c1)nc(-c1ccc(OCCC3CCCN3C)cc1Cl)n2Cc1ccccc1] | 1-benzyl-2-(2- chloro-4-(2- (1-methyl- pyrrolidin-2- yl)ethoxy) phenyl)-5- isopropoxy- 1H- benzo[d] imidazole (Isomer 1) | (500 MHz, CDCl₃): 1.36 (6H, d), 1.52-1.84 (3H, m), 1.96-2.04 (2H, m), 2.15- 2.22 (2H, m), 2.23-2.3 (1H, m), 2.35 (3H, s), 3.08 (1H, t), 3.96-4.12 (2H, m), 4.55 (1H, p), 5.20 (2H, s), 6.85 (2H, d), 6.94-7 (2H, m), 7.04 (1H, d), 7.06 (1H, d), 7.18-7.25 (3H, m), 7.32 (1H, d), 7.35 (1H, d). | 504 |
| A9 | 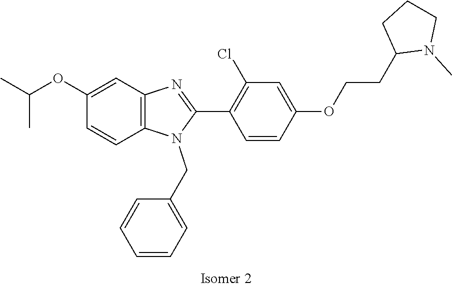 [SMILES: CC(C)Oc1ccc2c(c1)nc(-c1ccc(OCCC3CCCN3C)cc1Cl)n2Cc1ccccc1] | 1-benzyl-2-(2- chloro-4-(2- (1-methyl- pyrrolidin-2- yl)ethoxy) phenyl)-5- isopropoxy- 1H- benzo[d] imidazole (Isomer 2) | (500 MHz, CDCl₃): 1.36 (6H, d), 1.52-1.65 (1H, m), 1.71-1.83 (3H, m), 1.98- 2.04 (1H, m), 2.14-2.21 (2H, m), 2.22-2.29 (1H, m), 2.34 (3H, s), 3-3.13 (1H, m), 3.98-4.12 (2H, m), 4.55 (1H, p), 5.20 (2H, s), 6.85 (2H, d), 6.94-7 (2H, m), 7.04 (1H, d), 7.06 (1H, d), 7.19-7.25 (3H, m), 7.31 (1H, d), 7.35 (1H, d). | 504 |
| A10 | 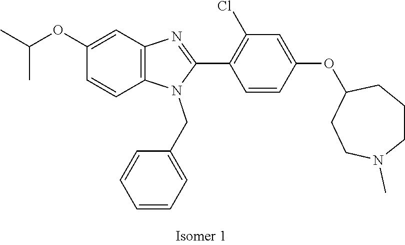 [SMILES: CC(C)Oc1ccc2c(c1)nc(-c1ccc(OC3CCCN(C)CC3)cc1Cl)n2Cc1ccccc1] | 1-benzyl-2-(2- chloro-4-((1- methylazepan- 4- yl)oxy) phenyl)-5- isopropoxy- 1H- benzo[d] imidazole (Isomer 1) | (500 MHz, CDCl₃): 1.36 (6H, d), 1.6-1.7 (1H, m), 1.82-1.93 (2H, m), 1.95- 2.03 (1H, m), 2.07-2.25 (2H, m), 2.37 (3H, s), 2.49- 2.66 (3H, m), 2.71 (1H, ddd), 4.51-4.65 (2H, m), 5.21 (2H, s), 6.81 (1H, dd), 6.84 (1H, dd), 6.95-6.99 (2H, m), 7.00 (1H, d), 7.05 (1H, d), 7.19-7.25 (3H, m), 7.32 (1H, d), 7.34 (1H, d). | 504 |
| A11 | 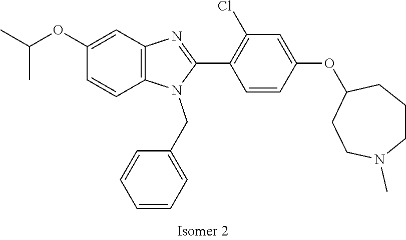 [SMILES: CC(C)Oc1ccc2c(c1)nc(-c1ccc(OC3CCCN(C)CC3)cc1Cl)n2Cc1ccccc1] | 1-benzyl-2-(2- chloro-4-((1- methylazepan- 4- yl)oxy) phenyl)-5- isopropoxy- 1H- benzo[d] imidazole (Isomer 2) | (500 MHz, CDCl₃): 1.36 (6H, d), 1.63-1.69 (1H, m), 1.82-1.93 (2H, m), 1.95- 2.03 (1H, m), 2.08-2.24 (2H, m), 2.37 (3H, s), 2.5- 2.66 (3H, m), 2.71 (1H, ddd), 4.51-4.62 (2H, m), 5.20 (2H, s), 6.80 (1H, dd), 6.84 (1H, dd), 6.96-6.99 (2H, m), 7.00 (1H, d), 7.05 (1H, d), 7.2-7.25 (3H, m), 7.31 (1H, d), 7.34 (1H, d). | 504 |
| A12 | 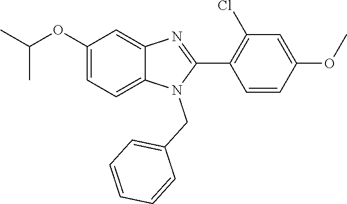 [SMILES: COc1ccc(-c2nc3cc(OC(C)C)ccc3n2Cc2ccccc2)c(Cl)c1] | 1-benzyl-2-(2- chloro-4- methoxy- phenyl)-5- isopropoxy- 1H- benzo[d] imidazole | (300 MHz, DMSO-d₆): 1.28 (6H, d), 3.86 (3H, s), 4.61 (1H, hept), 5.26 (2H, s), 6.84 (1H, dd), 6.97 (2H, dd), 7.06 (1H, dd), 7.15-7.26 (5H, m), 7.32 (1H, d), 7.50 (1H, d). | 407 |

[A3]3-(4-(1-benzyl-5-isopropoxy-1H-benzo[d]imidazol-2-yl)-3-chlorophenoxy)propan-1-amine (Synthetic Example 3) was acylated using acetyl chloride and DIEA in DCM.
[A4]3-(4-(1-benzyl-5-isopropoxy-1H-benzo[d]imidazol-2-yl)-3-chlorophenoxy)propan-1-amine (Synthetic Example 3) was acylated using heptanoyl chloride and DIEA in DCM.
[A5] made from a similar procedure as 2-(2-chloro-4-(2-(4-methylpiperazin-1-yl)ethoxy)phenyl)-1-(3-chlorobenzyl)-5-methoxy-1H-benzo[d]imidazole (Synthetic Example 2, intermediate), using N-(4-chlorobenzyl)-4-isopropoxy-2-nitroaniline and 4-methyl-6-(2-(4-methylpiperazin-1-yl)ethoxy)nicotinaldehyde as starting materials.
4-Methyl-6-(2-(4-methylpiperazin-1-yl)ethoxy)nicotinaldehyde was synthesised as follows:

Rockphos G3-Pd (113 mg, 0.12 mmol) was added slowly to 6-bromo-4-methylnicotinaldehyde (500 mg, 2.50 mmol), 2-(4-methylpiperazin-1-yl)ethan-1-ol (433 mg, 3.00 mmol) and Cₛ₂CO₃ (1629 mg, 5.00 mmol) in 1,4-dioxane (20 mL) at 20° C. under nitrogen. The resulting mixture was stirred at 100° C. for 18 hours. The reaction mixture was filtered through celite. The filtrate was concentrated under reduced pressure. The crude product was purified by flash C18-flash chromatography, elution gradient 5 to 80% MeCN in water (0.05% NH₄HCO₃). Pure fractions were evaporated to dryness to afford 4-methyl-6-(2-(4-methylpiperazin-1-yl)ethoxy)nicotinaldehyde (160 mg, 24%) as a yellow gum. ¹H NMR (300 MHz, CDCl₃): 2.33 (3H, s), 2.49-2.67 (11H, m), 2.81 (2H, t), 4.53 (2H, t), 6.61 (1H, s), 8.48 (1H, s), 10.05 (1H, s). One proton not observed. m/z: ES+[M+H]+ 264.

N-(4-chlorobenzyl)-4-isopropoxy-2-nitroaniline was made from a similar procedure to N4-benzyl-6-chloropyrimidine-4,5-diamine (Synthetic Example 1, intermediate) using 1-fluoro-4-isopropoxy-2-nitrobenzene and (4-chlorophenyl)methanamine. ¹H NMR (300 MHz, CDCl₃): 1.34 (6H, d), 4.47 (1H, q), 4.54 (2H, s), 6.71 (1H, d), 7.08 (1H, dd), 7.34 (3H, t), 7.70 (1H, d), 8.31 (1H, s). One proton not observable. m/z: ES+[M+H]+ 321.

1-Fluoro-4-isopropoxy-2-nitrobenzene was synthesised as follows:

DIAD (16.1 mL, 82.7 mmol) was added slowly to propan-2-ol (4.97 g, 82.7 mmol), 4-fluoro-3-nitrophenol (10 g, 63.6 mmol) and Ph₃P (25.04 g, 95.5 mmol) in THF (200 mL) at 0° C. under nitrogen. The resulting solution was stirred at rt for 18 hours. The solvent was removed under reduced pressure. The reaction mixture was diluted with EtOAc and petroleum ether (1:20, 100 mL). The solid was filtered out and the organic layer washed sequentially with saturated NH₄Cl (25 mL), saturated NaHCO₃ (25 mL) and saturated brine (2×25 mL). The organic layer was dried over Na₂SO₄, filtered and evaporated to afford crude product. The residue was purified by silica chromatography 0 to 20% EtOAc in petroleum ether to afford 1-fluoro-4-isopropoxy-2-nitrobenzene (10.00 g, 79%) as a yellow oil. ¹H NMR (400 MHz, DMSO-d₆): 1.27 (6H, d), 4.62-4.76 (1H, m), 7.31-7.39 (1H, m), 7.49 (1H, ddd), 7.57 (1H, dt).

[A6] made from a similar procedure as Synthetic Example A5, using N-(2-chloro-6-fluoro-3-methylbenzyl)-4-isopropoxy-2-nitroaniline and 2-chloro-4-(2-(4-methylpiperazin-1-yl)ethoxy)benzaldehyde as starting materials.

2-Chloro-4-(2-(4-methylpiperazin-1-yl)ethoxy)benzaldehyde was made following a procedure previous described in Synthetic Example 4.

N-(2-chloro-6-fluoro-3-methylbenzyl)-4-isopropoxy-2-nitroaniline was made using a similar procedure route as N-(4-chlorobenzyl)-4-isopropoxy-2-nitroaniline in Synthetic Example A5; using 1-fluoro-4-isopropoxy-2-nitrobenzene and (2-chloro-6-fluoro-3-methylphenyl)methanamine as starting materials. ¹H NMR (500 MHz, CDCl₃): 1.31 (6H, d), 2.36 (3H, s), 4.44 (1H, p), 4.65 (2H, dd), 6.94 (1H, t), 7.06 (1H, d), 7.11-7.21 (2H, m), 7.64 (1H, d), 8.27 (1H, t). m/z: ES+[M+H]+ 353.

[A7] made from a similar procedure as Synthetic Example A5, using N-(3-chlorobenzyl)-4-isopropoxy-2-nitroaniline and 4-methyl-6-(2-(4-methylpiperazin-1-yl)ethoxy)nicotinaldehyde as starting materials.

N-(3-chlorobenzyl)-4-isopropoxy-2-nitroaniline was made using a similar procedure route as N-(4-chlorobenzyl)-4-isopropoxy-2-nitroaniline in Synthetic Example A5; using (3-chlorophenyl)methanamine and 1-fluoro-4-isopropoxy-2-nitrobenzene as starting materials. ¹H NMR (300 MHz, DMSO-d₆): 1.20 (6H, d), 4.47 (1H, hept), 4.60 (2H, d), 6.83 (1H, d), 7.15-7.19 (1H, m), 7.37 (5H, m), 8.53 (1H, t). m/z: ES+[M+H]+ 321.

[A8] made from a similar procedure as 3-(4-(1-benzyl-5-isopropoxy-1H-benzo[d]imidazol-2-yl)-3-chlorophenoxy)propan-1-amine (Synthetic Example 3), using as starting materials benzyl-5-isopropoxy-1H-benzo[d]imidazol-2-yl)-3-chlorophenol and (rac)-2-(1-methylpyrrolidin-2-yl)ethan-1-ol, except that the deprotection step with TFA was omitted, to give a crude mixture containing (rac)-1-benzyl-2-(2-chloro-4-(2-(1-methylpyrrolidin-2-yl)ethoxy)phenyl)-5-isopropoxy-1H-benzo[d]imidazole and (rac)-1-benzyl-2-(2-chloro-4-((1-methylazepan-4-yl)oxy)phenyl)-5-isopropoxy-1H-benzo[d]imidazole. Examples A10 and A11 were obtained from purification of this crude mixture by chiral SFC (YMC SJ column, 20*250 mm, 5 micron) eluting with 10% MeOH (containing 0.1% NH₃) and 90% scCO₂ (60 mL/min, 120 bar, 40° C.) after evaporation of the solvents from pure fractions, to afford 1-benzyl-2-(2-chloro-4-((1-methylazepan-4-yl)oxy)phenyl)-5-isopropoxy-1H-benzo[d]imidazole, isomer 2 (Synthetic Example A11) (6.9 mg, 19%) and 1-benzyl-2-(2-chloro-4-((1-methylazepan-4-yl)oxy)phenyl)-5-isopropoxy-1H-benzo[d]imidazole, isomer 1 (Synthetic Example A10) (6.2 mg, 17%). Impure fractions containing a mixture of Examples A8 and A9 were further purified by chiral SFC (ChiralPak IC column, 20*250 mm, 5 micron) eluting with 45% MeOH (containing 0.1% NH₃) and 70% scCO₂ (60 mL/min, 120 bar, 40° C.) and evaporated to dryness to afford 1-benzyl-2-(2-chloro-4-(2-(1-methylpyrrolidin-2-yl)ethoxy)phenyl)-5-isopropoxy-1H-benzo[d]imidazole, isomer 1 (Synthetic Example A8) (9.4 mg, 26%) and 1-benzyl-2-(2-chloro-4-(2-(1-methylpyrrolidin-2-yl)ethoxy)phenyl)-5-isopropoxy-1H-benzo[d]imidazole, isomer 2 (Synthetic Example A9) (13.6 mg, 38%).

[A12] made from a similar procedure as 4-(1-benzyl-5-isopropoxy-1H-benzo[d]imidazol-2-yl)-3-chlorophenol, Synthetic Example A1, using N-benzyl-4-isopropoxy-2-nitroaniline and 2-chloro-4-methoxybenzaldehyde as starting materials.

The following examples in Table B were synthesised as stated in the notes following Table B.

TABLE B

| Syn- | | | | |
|---|---|---|---|---|
| thetic | | | | LCMS |
| Ex- | | | | [M + |
| ample | Structure | Name | ¹H NMR | H]+ |
| B1 | 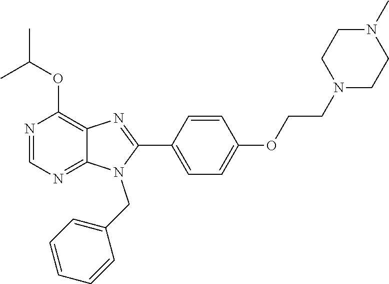 [SMILES: CC(C)Oc1ncnc2c1nc(-c1ccc(OCCN3CCN(C)CC3)cc1)n2Cc1ccccc1] | 9-benzyl-6- isopropoxy- 8- (4-(2-(4- methyl- piperazin- 1- yl)ethoxy) phenyl)- 9H-purine | (400 MHz, DMSO-d₆): 1.42 (6H, d), 2.16 (3H, s), 2.35 (8H, m), 2.69 (2H, t), 4.13 (2H, t), 5.56-5.65 (3H, m), 6.97-7.02 (2H, m), 7.03-7.07 (2H, m), 7.21-7.30 (3H, m), 7.64- 7.69 (2H, m), 8.50 (1H, s). | 487 |
| B2 | 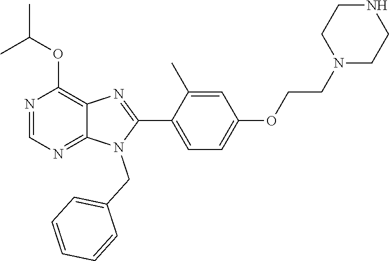 [SMILES: Cc1cc(OCCN2CCNCC2)ccc1-c1nc2c(OC(C)C)ncnc2n1Cc1ccccc1] | 9-benzyl-6- isopropoxy- 8- (2-methyl- 4-(2- (piperazin- 1- yl)ethoxy) phenyl)- 9H-purine | (500 MHz, CDCl₃): 1.49 (6H, d), 1.74 (1H, br s), 1.98 (3H, s), 2.51-2.64 (4H, m), 2.82 (2H, t), 2.88- 2.98 (4H, m), 4.15 (2H, t), 5.25 (2H, s), 5.69 (1H, hept), 6.76 (1H, dd), 6.81 (1H, d), 6.94 (2H, dd), 7.12 (1H, d), 7.15-7.24 (3H, m), 8.57 (1H, s). | 487 |
| B3 | 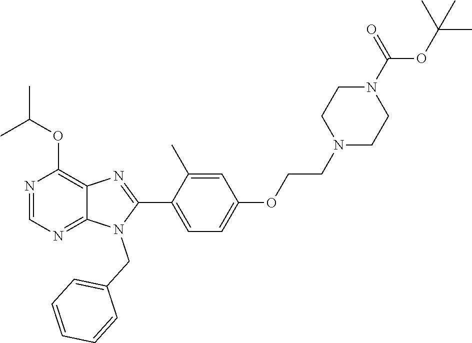 [SMILES: Cc1cc(OCCN2CCN(C(=O)OC(C)(C)C)CC2)ccc1-c1nc2c(OC(C)C)ncnc2n1Cc1ccccc1] | tert-butyl 4-(2- (4-(9- benzyl-6- isopropoxy- 9H- purin-8-yl)- 3-methyl- phenoxy) ethyl) piperazine- 1- carboxylate | (300 MHz, CDCl₃): 1.46- 1.53 (15H, m), 2.00 (3H, s), 2.58 (4H, s), 2.88 (2H, d), 3.50 (4H, s), 4.17 (2H, t), 5.26 (2H, s), 5.70 (1H, p), 6.72-6.85 (2H, m), 6.95 (2H, dd), 7.08-7.24 (4H, m), 8.58 (1H, s). | 587 |
| B4 | 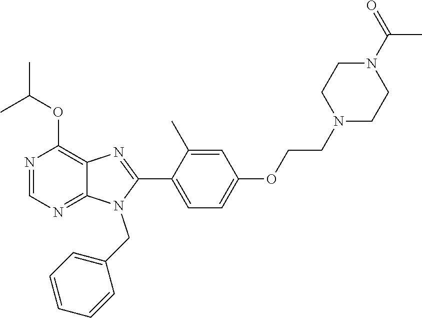 [SMILES: CC(=O)N1CCN(CCOc2ccc(-c3nc4c(OC(C)C)ncnc4n3Cc3ccccc3)c(C)c2)CC1] | 1-(4-(2- (4-(9- benzyl-6- isopropoxy- 9H- purin-8-yl)- 3-methyl- phenoxy) ethyl) piperazin- 1-yl)ethan- 1-one | (400 MHz, CD₃OD): 1.40- 1.52 (6H, m), 1.89 (3H, d), 2.04-2.18 (3H, m), 2.62 (4H, dt), 2.87 (2H, dd), 3.60 (4H, dt), 4.21 (2H, dd), 5.31 (2H, d), 5.70 (1H, ddd), 6.82-6.95 (4H, m), 7.13-7.26 (4H, m), 8.49-8.60 (1H, m). | 529 |
| B5 | 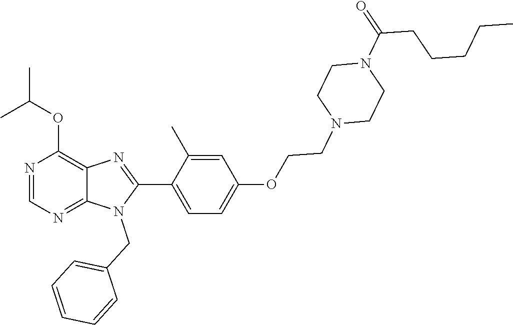 [SMILES: CCCCCC(=O)N1CCN(CCOc2ccc(-c3nc4c(OC(C)C)ncnc4n3Cc3ccccc3)c(C)c2)CC1] | 1-(4-(2- (4-(9- benzyl-6- isopropoxy- 9H- purin-8-yl)- 3-methyl- phenoxy) ethyl) piperazin- 1-yl)hexan- 1-one | (300 MHz, CDCl₃): 0.84- 0.95 (3H, m), 1.33 (4H, dt), 1.49 (6H, d), 1.64 (2H, t), 1.99 (3H, s), 2.25-2.40 (2H, m), 2.59 (4H, s), 2.86 (2H, t), 3.52 (2H, d), 3.68 (2H, s), 4.16 (2H, t), 5.25 (2H, s), 5.70 (1H, h), 6.72- 6.83 (2H, m), 6.90-6.98 (2H, m), 7.13 (1H, d), 7.15- 7.22 (3H, m), 8.57 (1H, s) | 585 |

[B1] made from a similar procedure as 9-benzyl-6-isopropoxy-8-(2-methyl-4-(2-(4-methylpiperazin-1-yl)ethoxy)phenyl)-9H-purine (Synthetic Example 1), using 2-(4-methylpiperazin-1-yl)ethan-1-ol and 4-(9-benzyl-6-isopropoxy-9H-purin-8-yl)phenol as starting materials.

4-(9-Benzyl-6-isopropoxy-9H-purin-8-yl)phenol was made using a similar procedure to 4-(9-benzyl-6-isopropoxy-9H-purin-8-yl)-3-methylphenol (Synthetic Example 1, intermediate) using 4-(9-benzyl-6-chloro-9H-purin-8-yl)phenol as the starting material. ¹H NMR (400 MHz, DMSO-d₆): 1.42 (6H, d), 1.99 (1H, s), 5.56 (2H, s), 6.83-6.88 (2H, m), 6.98-7.02 (2H, m), 7.26 (3H, dddd), 7.54-7.59 (2H, m), 8.48 (1H, s), 10.05 (1H, s). m/z: ES+[M+H]+ 361.

4-(9-Benzyl-6-chloro-9H-purin-8-yl)phenol was made using a similar procedure to 4-(9-benzyl-6-chloro-9H-purin-8-yl)-3-methylphenol (Synthetic Example 1, intermediate) using N4-benzyl-6-chloropyrimidine-4,5-diamine and 4-hydroxybenzaldehyde as starting materials. ¹H NMR (400 MHz, DMSO-d₆): 5.64 (2H, s), 6.87-6.92 (2H, m), 7.04-7.07 (2H, m), 7.24-7.31 (3H, m), 7.63-7.67 (2H, m), 8.75 (1H, s), 10.21 (1H, s). m/z: ES+[M+H]+ 337.

[B2] was made from tert-butyl 4-(2-(4-(9-benzyl-6-isopropoxy-9H-purin-8-yl)-3-methylphenoxy)ethyl)piperazine-1-carboxylate (40 mg, 0.07 mmol) using a similar procedure (BOC deprotection with TFA) as 9-benzyl-8-(2-chloro-4-(2-(piperazin-1-yl)ethoxy)phenyl)-6-((1,1,1-trifluoro-2-methylpropan-2-yl)oxy)-9H-purine (Synthetic Example 11) to afford 9-benzyl-6-isopropoxy-8-(2-methyl-4-(2-(piperazin-1-yl)ethoxy)phenyl)-9H-purine (18 mg, 53%) as a colourless dry film.

Tert-butyl 4-(2-(4-(9-benzyl-6-isopropoxy-9H-purin-8-yl)-3-methylphenoxy)ethyl)piperazine-1-carboxylate (Synthetic Example B3) was made using a similar procedure to 4-(1-benzyl-5-isopropoxy-1H-benzo[d]imidazol-2-yl)-3-chlorophenol (Synthetic Example A1, intermediate in Synthetic Example 3) using N-benzyl-6-isopropoxy-5-nitropyrimidin-4-amine and tert-butyl 4-(2-(4-formyl-3-methylphenoxy)ethyl)piperazine-1-carboxylate as starting materials.

Tert-butyl 4-(2-(4-formyl-3-methylphenoxy)ethyl)piperazine-1-carboxylate was made as follows:

4-Hydroxy-2-methylbenzaldehyde (2.0 g, 14.7 mmol), tert-butyl 4-(2-chloroethyl)piperazine-1-carboxylate (4.38 g, 17.6 mmol) and potassium carbonate (4.06 g, 29.4 mmol) in MeCN (20 mL) at 25° C. The resulting mixture was stirred at 80° C. for 2 hours. The reaction mixture was diluted with EtOAc (20 mL) and washed sequentially with water (3×20 mL). The organic layer was dried over Na₂SO₄, filtered and evaporated to afford crude product. Fractions were evaporated to dryness to afford tert-butyl 4-(2-(4-formyl-3-methylphenoxy)ethyl)piperazine-1-carboxylate (5.00 g, 98%) as a yellow gum. ¹H NMR (300 MHz, CDCl₃): 1.46 (9H, d), 2.60 (4H, d), 2.64 (3H, s), 2.88 (2H, s), 3.43-3.52 (4H, m), 4.21 (2H, s), 6.75 (1H, d), 6.81-6.87 (1H, m), 7.75 (1H, d), 10.12 (1H, s). m/z: ES+[M+H]+ 349.

N-Benzyl-6-isopropoxy-5-nitropyrimidin-4-amine was made as follows:

Sodium hydride (0.25 g, 6.35 mmol) was added portionwise to propan-2-ol (20 mL, 5.29 mmol) at 0° C. under nitrogen. The resulting solution was stirred at rt for 30 minutes. Then the solution was added dropwise into a solution of N-benzyl-6-chloro-5-nitropyrimidin-4-amine (1.4 g, 5.29 mmol) in propan-2-ol (20 mL, 5.29 mmol) and DMF (5 mL) at rt. The resulting solution was stirred at 60° C. for 18 hours. The reaction mixture was concentrated and diluted with EtOAc (250 mL), and washed sequentially with saturated NH₄Cl (50 mL), saturated NaHCO₃ (50 mL), and saturated brine (25 mL×3). The organic layer was dried over Na₂SO₄, filtered and evaporated to afford crude product. The crude product was purified by flash silica chromatography, elution gradient 0 to 10% EtOAc in petroleum ether. Pure fractions were evaporated to dryness to afford N-benzyl-6-isopropoxy-5-nitropyrimidin-4-amine (0.750 g, 49.2%) as a yellow oil which solidified on standing. ¹H NMR (300 MHz, DMSO-d₆): 1.32 (6H, d), 4.71 (2H, d), 5.42 (1H, h), 7.17-7.29 (1H, m), 7.32 (4H, d), 8.28 (1H, s), 8.89 (1H, t). m/z: ES+[M+H]+ 289.

[B4]9-benzyl-6-isopropoxy-8-(2-methyl-4-(2-(piperazin-1-yl)ethoxy)phenyl)-9H-purine (Synthetic Example B2) was acylated using acetyl chloride and DILA in DCM.

[B5]9-benzyl-6-isopropoxy-8-(2-methyl-4-(2-(piperazin-1-yl)ethoxy)phenyl)-9H-purine (Synthetic Example B2) was acylated using heptanoyl chloride and DIEA in DCM.

The following examples in Table C were synthesised using a similar procedure to that described in Synthetic Example 7, unless stated otherwise in the notes following Table C

TABLE C

| Synthetic | | | | LCMS |
|---|---|---|---|---|
| Example | Structure | Name | ¹H NMR | [M + H]+ |
| C1 | 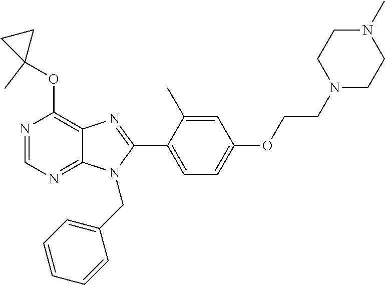 [SMILES: Cc1cc(OCCN2CCN(C)CC2)ccc1-c1nc2c(OC3(C)CC3)ncnc2n1Cc1ccccc1] | 9-benzyl-8-(2- methyl-4-(2-(4- methylpiperazin- 1- yl)ethoxy) phenyl)-6-(1- methylcyclopro- poxy)-9H-purine | (300 MHz, DMSO-d₆): 0.79- 0.85 (2H, m), 0.97-1.03 (2H, m), 1.71 (3H, s), 1.93 (3H, s), 2.13 (3H, s), 2.30 (4H, s), 2.46 (4H, s), 2.68 (2H, t), 4.10 (2H, t), 5.24 (2H, s), 6.83-6.89 (3H, m), 6.91 (1H, d), 7.17-7.22 (3H, m), 7.25 (1H, d), 8.58 (1H, s) | 513 |
| C2 | 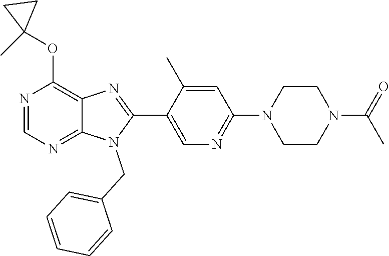 [SMILES: CC(=O)N1CCN(c2cc(C)c(-c3nc4c(OC5(C)CC5)ncnc4n3Cc3ccccc3)cn2)CC1] | 1-(4-(5-(9- benzyl-6-(1- methylcyclo- propoxy)- 9H-purin- 8-yl)-4- methylpyridin-2- yl)piperazin-1- yl)ethan-1-one | (400 MHz, DMSO-d₆): 0.82- 0.86 (2H, m), 1.00-1.04 (2H, m), 1.73 (3H, s), 1.98 (3H, s), 2.05 (3H, s), 3.50- 3.60 (6H, m), 3.60-3.70 (2H, m), 5.33 (2H, s), 6.82 (1H, s), 6.88-6.92 (2H, m), 7.20-7.26 (3H, m), 8.06 (1H, s), 8.59 (1H, s) | 498 |
| C3 | 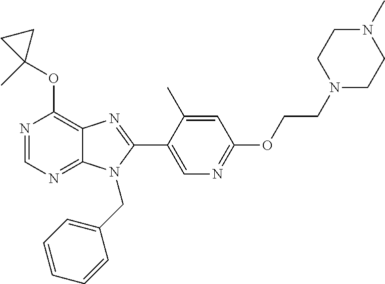 | 9-benzyl-8-(4- methyl-6-(2-(4- methylpiperazin- 1- yl)ethoxy) pyridin-3- yl)-6-(1- methylcyclo- propoxy)- 9H-purine | (300 MHz, DMSO-d₆): 0.80- 0.86 (2H, m), 0.98-1.03 (2H, m), 1.72 (3H, s), 1.90 (3H, s), 2.13 (3H, s), 2.24- 2.36 (4H, m), 2.44 (4H, br s), 2.66 (2H, t), 4.38 (2H, t), 5.31 (2H, s), 6.78 (1H, s), 6.84-6.89 (2H, m), 7.18- 7.21 (3H, m), 8.10 (1H, s), 8.61 (1H, s) | 514 |
| C4 | 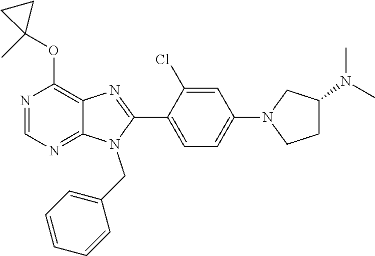 [SMILES: CN(C)[C@@H]1CCN(c2ccc(-c3nc4c(OC5(C)CC5)ncnc4n3Cc3ccccc3)c(Cl)c2)C1] | (R)-1-(4-(9- benzyl-6-(1- methylcyclo- propoxy)- 9H-purin- 8-yl)-3- chlorophenyl)- N,N- dimethyl- pyrrolidin- 3-amine | (300 MHz, DMSO-d₆): 0.79- 0.85 (2H, m), 0.99 (2H, d), 1.71 (3H, s), 1.83 (1H, q), 2.21 (7H, s), 2.81 (1H, s), 3.08 (1H, t), 3.22-3.29 (1H, m), 3.38-3.55 (2H, m), 5.28 (2H, s), 6.56 (1H, dd), 6.70 (1H, d), 6.87- 6.94 (2H, m), 7.16-7.27 (4H, m), 8.55 (1H, s). | 503 |
| C5 | 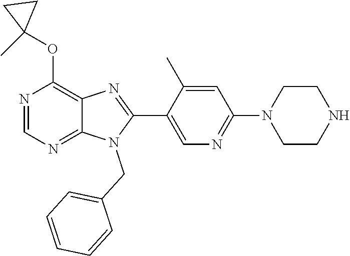 [SMILES: Cc1cc(N2CCNCC2)ncc1-c1nc2c(OC3(C)CC3)ncnc2n1Cc1ccccc1] | 9-benzyl-8-(4- methyl-6- (piperazin-1- yl)pyridin-3-yl)- 6-(1- methylcyclo- propoxy)- 9H-purine | (300 MHz, DMSO-d₆): 0.77- 0.87 (2H, m), 0.90-1.10 (2H, m), 1.71 (3H, s), 1.95 (3H, s), 2.75 (4H, t), 3.47 (4H, t), 5.31 (2H, s), 6.73 (1H, s), 6.84-6.93 (2H, m), 7.17-7.25 (3H, m), 8.01 (1H, s), 8.56 (1H, s). 1H not observed. | 456 |
| C6 | 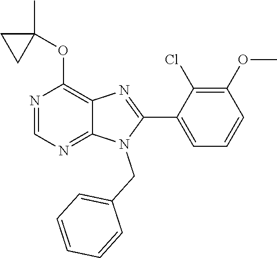 [SMILES: COc1cccc(-c2nc3c(OC4(C)CC4)ncnc3n2Cc2ccccc2)c1Cl] | 9-benzyl-8-(2- chloro-3- methoxyphenyl)- 6-(1- methylcyclo- propoxy)- 9H-purine | (300 MHz, DMSO-d₆): 0.83 (2H, t), 1.00 (2H, t), 1.71 (3H, s), 3.91 (3H, s), 5.25 (2H, s), 6.83-6.93 (2H, m), 7.03 (1H, dd), 7.12-7.23 (3H, m), 7.32-7.46 (2H, m), 8.61 (1H, s). | 421 |
| C7 | 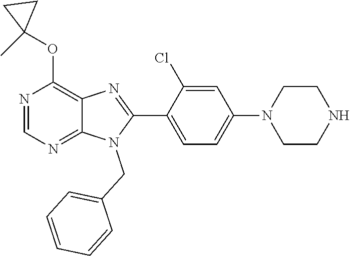 [SMILES: CC1(Oc2ncnc3c2nc(-c2ccc(N4CCNCC4)cc2Cl)n3Cc2ccccc2)CC1] | 9-benzyl-8-(2- chloro-4- (piperazin-1- yl)phenyl)-6-(1- methylcyclo- propoxy)- 9H-purine | (300 MHz, DMSO-d₆): 0.77- 0.87 (2H, m), 0.90-1.05 (2H, m), 1.71 (3H, s), 2.80 (4H, t), 3.18 (4H, t), 5.28 (2H, s), 6.85-6.98 (3H, m), 7.06 (1H, d), 7.19 (3H, dd), 7.26 (1H, d), 8.57 (1H, s). 1H not observed. | 475 |
| C8 | 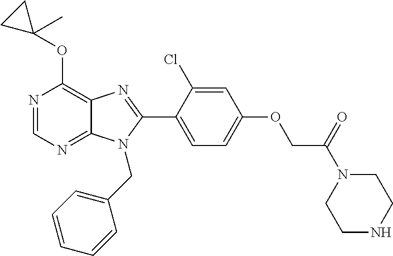 [SMILES: CC1(Oc2ncnc3c2nc(-c2ccc(OCC(=O)N4CCNCC4)cc2Cl)n3Cc2ccccc2)CC1] | 2-(4-(9-benzyl- 6-(1- methylcyclo- propoxy)- 9H-purin- 8-yl)-3- chlorophenoxy)- 1-(piperazin-1- yl)ethan-1-one | (400 MHz, DMSO-d₆): 0.81- 0.89 (2H, m), 0.99-1.06 (2H, m), 1.73 (3H, s), 2.65 (2H, d), 2.71 (2H, s), 3.37 (4H, s), 4.97 (2H, s), 5.28 (2H, s), 6.85-6.93 (2H, m), 7.01 (1H, dd), 7.15-7.28 (4H, m), 7.41 (1H, d), 8.61 (1H, s). 1H not observed. | 533 |
| C9 | 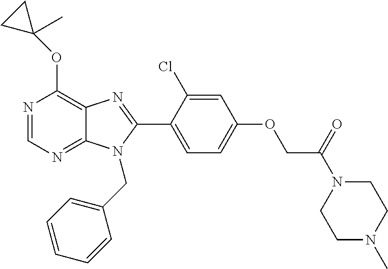 [SMILES: CN1CCN(C(=O)COc2ccc(-c3nc4c(OC5(C)CC5)ncnc4n3Cc3ccccc3)c(Cl)c2)CC1] | 2-(4-(9-benzyl- 6-(1- methylcyclo- propoxy)- 9H-purin- 8-yl)-3- chlorophenoxy)- 1-(4- methylpiperazin- 1-yl)ethan-1-one | (400 MHz, DMSO-d₆): 0.80- 0.88 (2H, m), 0.98-1.06 (2H, m), 1.73 (3H, s), 2.19 (3H, s), 2.27 (2H, t), 2.34 (2H, s), 3.45 (4H, s), 4.99 (2H, s), 5.28 (2H, s), 6.85- 6.94 (2H, m), 7.01 (1H, dd), 7.16-7.26 (4H, m), 7.41 (1H, d), 8.61 (1H, s). | 547 |

[C1] made from a similar procedure as 4-((8-(2-chloro-4-(2-(4-methylpiperazin-1-yl)ethoxy)phenyl)-6-(1-methylcyclopropoxy)-9H-purin-9-yl)methyl)-2-methylthiazole (Synthetic Example 7), using 2-methyl-4-(2-(4-methylpiperazin-1-yl)ethoxy)benzaldehyde and N-benzyl-6-(1-methylcyclopropoxy)-5-nitropyrimidin-4-amine (Synthetic Example 4, intermediate) as starting materials. Product obtained as a white solid (23 mg, 13%).

2-Methyl-4-(2-(4-methylpiperazin-1-yl)ethoxy)benzaldehyde was made by a similar procedure as 9-benzyl-8-(2-chloro-4-((1-methylpiperidin-4-yl)methoxy)phenyl)-6-(1-methylcyclopropoxy)-9H-purine (Synthetic Example 5), using 4-hydroxy-2-methylbenzaldehyde and 2-(4-methylpiperazin-1-yl)ethan-1-ol as starting materials. Product obtained as a yellow gum, (0.23 g, 30%). ¹H NMR (300 MHz, DMSO-d₆): 2.12 (3H, s), 2.23-2.45 (8H, m), 2.56 (3H, s), 2.67 (2H, t), 4.15 (2H, t), 6.89 (1H, d), 6.94 (1H, dd), 7.75 (1H, d), 10.05 (1H, s). m/z: ES+[M+H]+ 263.

[C2] made in a similar manner to that described in Synthetic Example 7 using benzyl-6-(1-methylcyclopropoxy)-5-nitropyrimidin-4-amine (150 mg, 0.50 mmol) and 6-(4-acetylpiperazin-1-yl)-4-methylnicotinaldehyde (247 mg, 1.00 mmol) to afford 1-(4-(5-(9-benzyl-6-(1-methylcyclopropoxy)-9H-purin-8-yl)-4-methylpyridin-2-yl)piperazin-1-yl)ethan-1-one (42 mg, 17%) as a light yellow solid.

6-(4-Acetylpiperazin-1-yl)-4-methylnicotinaldehyde used as starting material was made as follows:

### 6-(4-Acetylpiperazin-1-yl)-4-methylnicotinaldehyde

6-Chloro-4-methylnicotinaldehyde (0.50 g, 3.21 mmol), 1-(piperazin-1-yl)ethan-1-one (0.824 g, 6.43 mmol) and K₂CO₃ (1.33 g, 9.64 mmol) in DMA (5 mL) were stirred under an atmosphere of nitrogen at 100° C. for 3 hours. The reaction mixture was diluted with saturated NH₄Cl (50 mL) and extracted with EtOAc (50 mL×3). The organic layer was washed with brine (50 mL×3), dried over Na₂SO₄, filtered and evaporated to afford 6-(4-acetylpiperazin-1-yl)-4-methylnicotinaldehyde (0.45 g, 57%) as a yellow gum. The product was used in the next step directly without further purification. ¹H NMR (300 MHz, DMSO-d₆): 2.03 (3H, s), 2.45 (3H, s), 3.49-3.56 (4H, m), 3.72 (4H, ddd), 6.73 (1H, s), 8.46 (1H, s), 9.84 (1H, s). m/z: ES+[M+H]+ 248.

[C3] made in a similar manner to that described in Synthetic Example 7 using N-benzyl-6-(1-methylcyclopropoxy)-5-nitropyrimidin-4-amine (120 mg, 0.40 mmol) and 4-methyl-6-(2-(4-methylpiperazin-1-yl)ethoxy)nicotinaldehyde (210 mg, 0.80 mmol) to afford benzyl-8-(4-methyl-6-(2-(4-methylpiperazin-1-yl)ethoxy)pyridin-3-yl)-6-(1-methylcyclopropoxy)-9H-purine (32 mg, 16%) as a light yellow solid.

[C4] made via a similar method to 4-((8-(2-chloro-4-(2-(4-methylpiperazin-1-yl)ethoxy)phenyl)-6-(1-methylcyclopropoxy)-9H-purin-9-yl)methyl)-2-methylthiazole (Synthetic Example 7), using N-benzyl-6-(1-methylcyclopropoxy)-5-nitropyrimidin-4-amine (intermediate in Synthetic Example 4) and (R)-2-chloro-4-(3-(dimethylamino)pyrrolidin-1-yl)benzaldehyde.

(R)-2-chloro-4-(3-(dimethylamino)pyrrolidin-1-yl)benzaldehyde was obtained as follows:

2-chloro-4-fluorobenzaldehyde (0.3 g, 1.89 mmol), (R)-N,N-dimethylpyrrolidin-3-amine (0.432 g, 3.78 mmol) and K₂CO₃ (0.784 g, 5.68 mmol) in DMA (5 mL) were stirred under an atmosphere of nitrogen at 100° C. for 16 hours. The reaction mixture was filtered. The reaction mixture was diluted with saturated NH₄Cl (50 mL) and extracted with EtOAc (3×50 mL). The organic layer was washed with brine (3×50 mL), dried over Na₂SO₄, filtered and evaporated to afford a yellow solid. The product was used in the next step directly without further purification. m/z: ES+[M+H]+ 253

[C5] made in a similar manner to that described in Synthetic Example 7 using N-benzyl-6-(1-methylcyclopropoxy)-5-nitropyrimidin-4-amine (150 mg, 0.50 mmol) and tert-butyl 4-(5-formyl-4-methylpyridin-2-yl)piperazine-1-carboxylate (305 mg, 1.00 mmol) to afford 9-benzyl-8-(4-methyl-6-(piperazin-1-yl)pyridin-3-yl)-6-(1-methylcyclopropoxy)-9H-purine (31 mg, 14%) as a light yellow solid.

Tert-butyl 4-(5-formyl-4-methylpyridin-2-yl)piperazine-1-carboxylate used as a starting material was made as follows:

### Tert-butyl 4-(5-formyl-4-methylpyridin-2-yl)piperazine-1-carboxylate

6-Chloro-4-methylnicotinaldehyde (0.5 g, 3.21 mmol), tert-butyl piperazine-1-carboxylate (1.20 g, 6.43 mmol) and K₂CO₃ (1.33 g, 9.64 mmol) in DMA (5 mL) were stirred under an atmosphere of nitrogen at 100° C. for 16 hours. The reaction mixture was diluted with saturated aq. NH₄Cl (50 mL) and extracted with EtOAc (50 mL×3). The organic layer was washed with brine (50 mL×3), dried over Na₂SO₄, filtered and evaporated to afford tert-butyl 4-(5-formyl-4-methylpyridin-2-yl)piperazine-1-carboxylate (0.60 g, 61%) as a black gum. The product was used in the next step directly without further purification. ¹H NMR (300 MHz, DMSO-d₆): 1.43 (9H, s), 1.96 (3H, s), 2.36 (2H, t), 3.30 (2H, t), 3.41-3.44 (2H, m), 3.70-3.73 (2H, m), 6.74 (1H, s), 8.47 (1H, s), 9.85 (1H, s). m/z: ES+[M+H]+ 306.

[C6] made in a similar manner to that described in Synthetic Example 7 using N-benzyl-6-(1-methylcyclopropoxy)-5-nitropyrimidin-4-amine (150 mg, 0.50 mmol) and 2-chloro-3-methoxybenzaldehyde (256 mg, 1.50 mmol) to afford 9-benzyl-8-(2-chloro-3-methoxyphenyl)-6-(1-methylcyclopropoxy)-9H-purine (43 mg, 20%) as a light yellow solid.

[C7] was made using a similar procedure to 4-((8-(2-chloro-4-(2-(4-methylpiperazin-1-yl)ethoxy)phenyl)-6-(1-methylcyclopropoxy)-9H-purin-9-yl)methyl)-2-methylthiazole (Synthetic Example 7), using N-benzyl-6-(1-methylcyclopropoxy)-5-nitropyrimidin-4-amine (Intermediate in Synthetic Example 4) and tert-butyl 4-(3-chloro-4-formylphenyl)piperazine-1-carboxylate.

Tert-butyl 4-(3-chloro-4-formylphenyl)piperazine-1-carboxylate was obtained as follows:

2-Chloro-4-fluorobenzaldehyde (0.5 g, 3.15 mmol), tert-butyl piperazine-1-carboxylate (0.587 g, 3.15 mmol) and K₂CO₃ (1.31 g, 9.46 mmol) in acetonitrile (10 mL) were stirred under an atmosphere of nitrogen at 100° C. for 16 hours. The solvent was then removed under reduced pressure. The reaction mixture was diluted with saturated NH₄Cl (50 mL) and extracted with EtOAc (3×50 mL). The organic layers were washed with brine (100 mL), dried over Na₂SO₄, filtered and evaporated to afford tert-butyl 4-(3-chloro-4-formylphenyl)piperazine-1-carboxylate (0.80 g, 78%) as a yellow gum. The product was used in the next step directly without further purification. m/z: ES+[M+H]+ 325

[C8] was made using a similar procedure to 4-((8-(2-chloro-4-(2-(4-methylpiperazin-1-yl)ethoxy)phenyl)-6-(1-methylcyclopropoxy)-9H-purin-9-yl)methyl)-2-methylthiazole (Synthetic Example 7), using N-benzyl-6-(1-methylcyclopropoxy)-5-nitropyrimidin-4-amine (Intermediate in Synthetic Example 4) and tert-butyl 4-(2-(3-chloro-4-formylphenoxy)acetyl)piperazine-1-carboxylate.

Tert-butyl 4-(2-(3-chloro-4-formylphenoxy)acetyl)piperazine-1-carboxylate was obtained as follows:

K₂CO₃ (0.883 g, 6.39 mmol) was added to tert-butyl 4-(2-chloroacetyl)piperazine-1-carboxylate (1.01 g, 3.83 mmol) and 2-chloro-4-hydroxybenzaldehyde (0.5 g, 3.19 mmol) in DMF (5 mL). The resulting mixture was stirred at 80° C. for 1 hour. The reaction mixture was diluted with saturated brine (50 mL) and extracted with EtOAc (3×50 mL). The organic layers were dried over Na₂SO₄, filtered and evaporated to afford crude tert-butyl 4-(2-(3-chloro-4-formylphenoxy)acetyl)piperazine-1-carboxylate (1.2 g, 98%) as a yellow solid. The product was used in the next step directly without further purification. m/z: ES+[M-tBu]+327

[C9] Sodium triacetoxyborohydride (238.5 mg, 1.13 mmol) was added to 2-(4-(9-benzyl-6-(1-methylcyclopropoxy)-9H-purin-8-yl)-3-chlorophenoxy)-1-(piperazin-1-yl)ethan-1-one (Synthetic Example C8, 150 mg, 0.28 mmol) in 37% aqueous formaldehyde solution (2 mL). The resulting mixture was stirred at 25° C. for 1 hour. The solvent was removed under reduced pressure. The crude product was purified by flash C18-flash chromatography, elution gradient 5 to 100% MeCN in water (containing 0.1% NaHCO₃). Desired fractions were evaporated to dryness and purified further by preparative HPLC, column: XBridge Prep OBD C18 Column, 30*150 mm, 5 μm; using decreasingly polar mixtures of acetonitrile in water (0.1% aq. NH₃ and 10 mmol/L NH₄HCO₃). Fractions containing the desired compound were evaporated to dryness to afford 2-(4-(9-benzyl-6-(1-methylcyclopropoxy)-9H-purin-8-yl)-3-chlorophenoxy)-1-(4-methylpiperazin-1-yl)ethan-1-one (41 mg, 27%) as a brown solid.

The following examples in Table D were synthesised as described in the notes following Table D. Otherwise, a similar method to Synthetic Example D1 was used

TABLE D

| | | | | LC |
|---|---|---|---|---|
| | | | | MS |
| Syn | | | | [M + |
| Ex # | Structure | Name | ¹H NMR | H]+ |
| D1 | 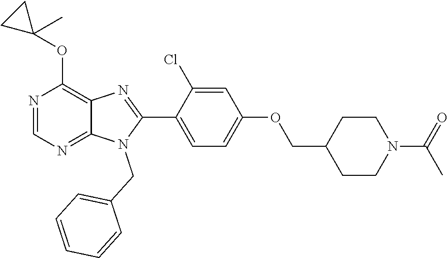 [SMILES: CC(=O)N1CCC(COc2ccc(-c3nc4c(OC5(C)CC5)ncnc4n3Cc3ccccc3)c(Cl)c2)CC1] | 1-(4-((4- (9-benzyl- 6-(1- methylcyclo- propoxy)- 9H-purin- 8-yl)-3- chloro- phenoxy) methyl) piperidin- 1-yl)ethan- 1-one | (400 MHz, DMSO-d₆): 0.80- 0.92 (2H, m), 0.94-1.10 (2H, m), 1.07-1.19 (1H, m), 1.26 (1H, qd), 1.73 (3H, s), 1.79 (2H, td), 1.95-2.05 (4H, m), 2.56 (1H, td), 3.06 (1H, td), 3.85 (1H, d), 3.96 (2H, d), 4.41 (1H, d), 5.29 (2H, s), 6.86- 6.95 (2H, m), 7.03 (1H, dd), 7.16-7.26 (4H, m), 7.41 (1H, d), 8.61 (1H, s). | 546 |
| D2 | 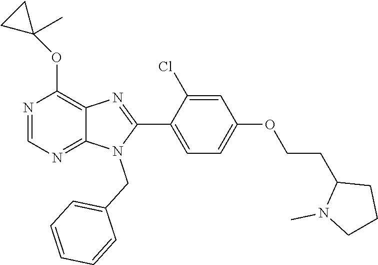 [SMILES: CN1CCCC1CCOc1ccc(-c2nc3c(OC4(C)CC4)ncnc3n2Cc2ccccc2)c(Cl)c1] | 9-benzyl- 8-(2- chloro- 4-(2-(1- methyl- pyrrolidin-2- yl)ethoxy) phenyl)- 6-(1- methylcyclo- propoxy)- 9H-purine | (400 MHz, DMSO-d₆): 0.80- 0.92 (2H, m), 0.94-1.06 (2H, m), 1.49 (1H, dtd), 1.58- 1.71 (3H, m), 1.73 (3H, s), 1.86-2.00 (1H, m), 2.01-2.14 (2H, m), 2.20 (1H, dt), 2.24 (3H, s), 2.95 (1H, ddd), 4.12 (2H, t), 5.29 (2H, s), 6.85- 6.94 (2H, m), 7.02 (1H, dd), 7.15-7.24 (4H, m), 7.40 (1H, d), 8.61 (1H, s). | 518 |
| D3 | 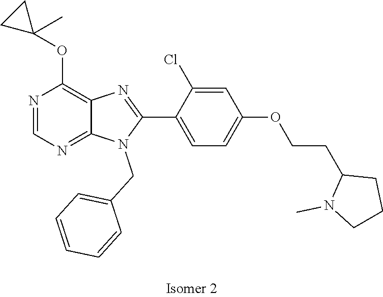 [SMILES: CN1CCCC1CCOc1ccc(-c2nc3c(OC4(C)CC4)ncnc3n2Cc2ccccc2)c(Cl)c1] | 9-benzyl- 8-(2- chloro- 4-(2-(1- methyl- pyrrolidin-2- yl)ethoxy) phenyl)- 6-(1- methylcyclo- propoxy)- 9H-purine (Isomer 2) | (500 MHz, DMSO-d₆): 0.81- 0.86 (2H, m), 1-1.04 (2H, m), 1.45-1.54 (1H, m), 1.59- 1.68 (3H, m), 1.73 (3H, s), 1.88-1.96 (1H, m), 2.03-2.1 (2H, m), 2.15-2.21 (1H, m), 2.23 (3H, s), 2.92-2.97 (1H, m), 4.11 (2H, t), 5.29 (2H, s), 6.88-6.91 (2H, m), 7.02 (1H, dd), 7.18-7.23 (4H, m), 7.40 (1H, d), 8.61 (1H, s). | 518 |
| D4 | 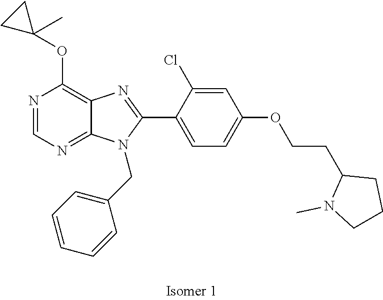 [SMILES: CN1CCCC1CCOc1ccc(-c2nc3c(OC4(C)CC4)ncnc3n2Cc2ccccc2)c(Cl)c1] | 9-benzyl- 8-(2- chloro- 4-(2-(1- methyl- pyrrolidin-2- yl)ethoxy) phenyl)- 6-(1- methylcyclo- propoxy)- 9H-purine (Isomer 1) | (500 MHz, DMSO-d₆): 0.81- 0.86 (2H, m), 0.99-1.05 (2H, m), 1.44-1.53 (1H, m), 1.59-1.67 (3H, m), 1.72 (3H, s), 1.88-1.97 (1H, m), 2.02-2.10 (2H, m), 2.19 (1H, dq), 2. 23 (3H, s), 2.9-2.97 (1H, m), 4.11 (2H, t), 5.28 (2H, s), 6.89 (2H, d), 7.01 (1H, dd), 7.16-7.23 (4H, m), 7.39 (1H, d), 8.61 (1H, s). | 518 |
| D5 | 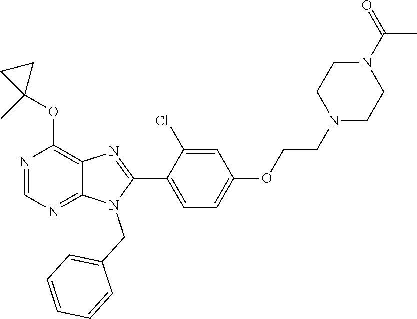 [SMILES: CC(=O)N1CCN(CCOc2ccc(-c3nc4c(OC5(C)CC5)ncnc4n3Cc3ccccc3)c(Cl)c2)CC1] | 1-(4-(2-(4-(9- benzyl-6-(1- methylcyclo- propoxy)- 9H-purin- 8-yl)-3- chloro- phenoxy) ethyl) piperazin-1- yl)ethan- 1-one | (400 MHz, DMSO-d₆): 0.82- 0.87 (2H, t), 1.00-1.05 (2H, t), 1.73 (3H, s), 1.99 (3H, s), 2.44 (2H, t), 2.53 (2H, d), 2.75 (2H, t), 3.43 (4H, q), 4.21 (2H, t), 5.29 (2H, s), 6.88-6.94 (2H, m), 7.05 (1H, dd), 7.18- 7.23 (3H, m), 7.27 (1H, d), 7.42 (1H, d), 8.62 (1H, s). | 561 |
| D6 | 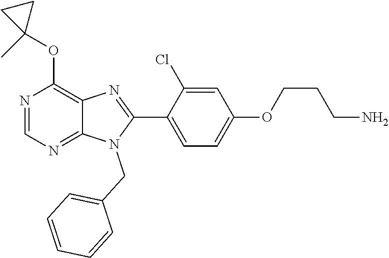 [SMILES: CC1(Oc2ncnc3c2nc(-c2ccc(OCCCN)cc2Cl)n3Cc2ccccc2)CC1] | 3-(4-(9- benzyl-6- (1- methyl- cyclopro- poxy)- 9H-purin- 8-yl)-3- chloro- phenoxy) propan-1- amine | (300 MHz, DMSO-d₆): 0.82- 0.87 (2H, t), 1.03 (2H, t), 1.73 (3H, s), 1.81 (2H, m), 1.94 (2H, m), 2.71 (2H, t), 4.14 (2H, t), 5.29 (2H, s), 6.88-6.94 (2H, m), 7.03 (1H, m), 7.21 (4H, m), 7.41 (1H, d), 8.62 (1H, s) | 464 |
| D7 | 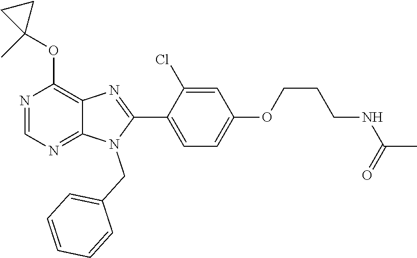 [SMILES: CC(=O)NCCCOc1ccc(-c2nc3c(OC4(C)CC4)ncnc3n2Cc2ccccc2)c(Cl)c1] | N-(3-(4- (9-benzyl- 6-(1- methylcyclo- propoxy)- 9H-purin- 8-yl)-3- chloro- phenoxy) propyl) acetamide | (300 MHz, DMSO-d₆): 0.80- 0.87 (2H, t), 1.02 (2H, t), 1.74 (3H, s), 1.81 (3H, s), 1.87 (2H, m), 3.21 (2H, m), 4.10 (2H, t), 5.30 (2H, s), 6.91 (2H, m), 7.03 (1H, m), 7.21 (4H, m), 7.43 (1H, d), 7.93 (1H, t), 8.62 (1H, s) | 506 |
| D8 | 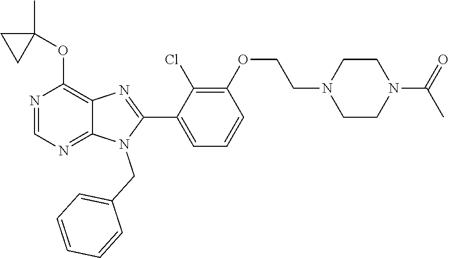 [SMILES: CC(=O)N1CCN(CCOc2cccc(-c3nc4c(OC5(C)CC5)ncnc4n3Cc3ccccc3)c2Cl)CC1] | 1-(4-(2-(3-(9- benzyl-6-(1- methylcyclo- propoxy)- 9H-purin- 8-yl)-2- chloro- phenoxy) ethyl) piperazin-1- yl)ethan- 1-one | (300 MHz, DMSO-d₆): 0.84 (2H, d), 1.01 (2H, q), 1.71 (3H, s), 1.96 (3H, s), 2.44 (2H, d), 2.53 (2H, d), 2.78 (2H, t), 3.40 (4H, q), 4.25 (2H, t), 5.25 (2H, s), 6.83-6.92 (2H, m), 7.03 (1H, dd), 7.12- 7.22 (3H, m), 7.35-7.43 (2H, m), 8.61 (1H, s) | 561 |
| D9 | 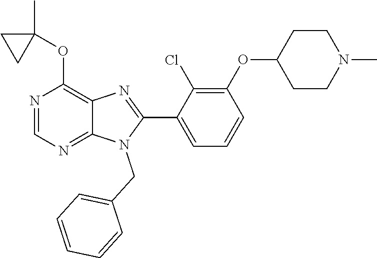 [SMILES: CN1CCC(Oc2cccc(-c3nc4c(OC5(C)CC5)ncnc4n3Cc3ccccc3)c2Cl)CC1] | 9-benzyl- 8-(2- chloro-3-((1- methyl- piperidin-4- yl)oxy) phenyl)-6- (1- methylcyclo- propoxy)- 9H-purine | (300 MHz, DMSO-d₆): 0.78- 0.87 (2H, m), 1.00 (2H, d), 1.72 (5H, s), 1.91 (2H, s), 2.16 (3H, s), 2.21 (2H, s), 2.59 (2H, s), 4.55 (1H, s), 5.25 (2H, s), 6.86 (2H, dd), 7.02 (1H, dd), 7.17 (3H, dd), 7.32- 7.45 (2H, m), 8.61 (1H, s). | 504 |
| D10 | 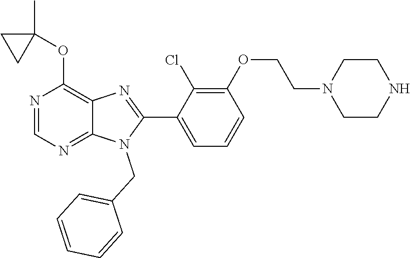 [SMILES: CC1(Oc2ncnc3c2nc(-c2cccc(OCCN4CCNCC4)c2Cl)n3Cc2ccccc2)CC1] | 9-benzyl- 8-(2- chloro-3-(2- (piperazin-1- yl)ethoxy) phenyl)- 6-(1- methylcyclo- propoxy)- 9H-purine | (300 MHz, DMSO-d₆): 0.78- 0.86 (2H, m), 0.96-1.06 (2H, m), 1.71 (3H, s), 2.41 (4H, s), 2.67 (6H, dd), 4.22 (2H, t), 5.25 (2H, s), 6.84-6.92 (2H, m), 7.02 (1H, dd), 7.14- 7.21 (3H, m), 7.38 (2H, d), 8.61 (1H, s). One proton not observed. | 519 |
| D11 | 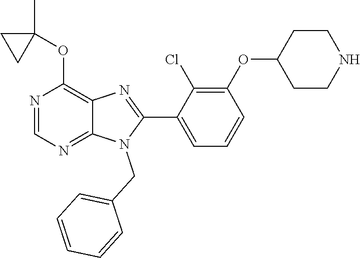 [SMILES: CC1(Oc2ncnc3c2nc(-c2cccc(OC4CCNCC4)c2Cl)n3Cc2ccccc2)CC1] | 9-benzyl- 8-(2- chloro-3- (piperidin-4- yloxy) phenyl)-6- (1- methylcyclo- propoxy)- 9H-purine | (300 MHz, DMSO-d₆): 0.79- 0.87 (2H, m), 0.98-1.05 (2H, m), 1.53 (2H, dd), 1.72 (3H, s), 1.88 (2H, s), 2.60 (2H, d), 2.94 (2H, dd), 4.59 (1H, d), 5.26 (2H, s), 6.86 (2H, dd), 7.02 (1H, dd), 7.17 (3H, dd), 7.32-7.46 (2H, m), 8.61 (1H, s). One proton not observed. | 490 |
| D12 | 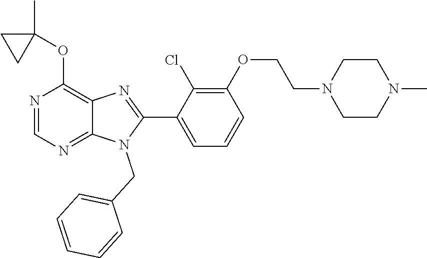 [SMILES: CN1CCN(CCOc2cccc(-c3nc4c(OC5(C)CC5)ncnc4n3Cc3ccccc3)c2Cl)CC1] | 9-benzyl- 8-(2- chloro- 3-(2-(4- methyl- piperazin-1- yl)ethoxy) phenyl)- 6-(1- methylcyclo- propoxy)- 9H-purine | (300 MHz, DMSO-d₆): 0.78- 0.87 (2H, m), 0.97-1.05 (2H, m), 1.71 (3H, s), 2.14 (3H, s), 2.32 (4H, s), 2.52 (4H, d), 2.74 (2H, d), 4.22 (2H, t), 5.25 (2H, s), 6.84-6.91 (2H, m), 7.02 (1H, dd), 7.14- 7.21 (3H, m), 7.38 (2H, d), 8.61 (1H, s). | 533 |
| D13 | 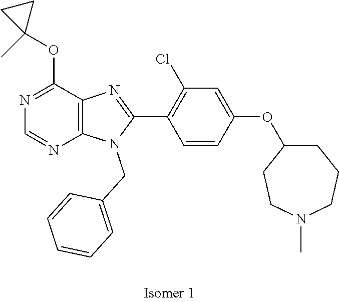 [SMILES: CN1CCCC(Oc2ccc(-c3nc4c(OC5(C)CC5)ncnc4n3Cc3ccccc3)c(Cl)c2)CC1] | 9-benzyl- 8-(2- chloro-4-((1- methyl- azepan-4- yl)oxy) phenyl)-6- (1- methylcyclo- propoxy)- 9H-purine (Isomer 1) | (400 MHz, DMSO-d₆): 0.81- 0.88 (2H, t), 1.02 (2H, t), 1.59 (1H, m), 1.70-1.89 (6H, m), 2.07 (2H, m), 2.26 (3H, s), 2.42-2.49 (2H, m), 2.53-2.65 (2H, m), 4.74 (1H, m), 5.29 (2H, s), 6.90 (2H, m), 6.98 (1H, m), 7.15-7.22 (4H, m), 7.38 (1H, d), 8.62 (1H, s) | 518 |
| D14 | 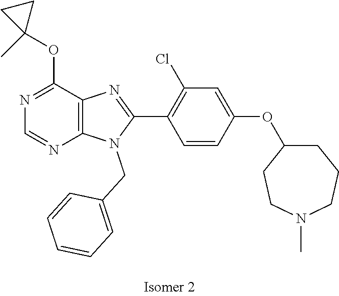 [SMILES: CN1CCCC(Oc2ccc(-c3nc4c(OC5(C)CC5)ncnc4n3Cc3ccccc3)c(Cl)c2)CC1] | 9-benzyl- 8-(2- chloro-4-((1- methyl- azepan-4- yl)oxy) phenyl)-6- (1- methylcyclo- propoxy)- 9H-purine (Isomer 2) | (400 MHz, DMSO-d₆): 0.81- 0.88 (2H, t), 1.02 (2H, t), 1.53- 1.64 (1H, m), 1.69-1.90 (6H, m), 2.00-2.14 (2H, m), 2.26 (3H, s), 2.45 (2H, m), 2.53-2.65 (2H, m), 4.75 (1H, m), 5.29 (2H, s), 6.87- 6.93 (2H, m), 6.98 (1H, m), 7.16-7.22 (4H, m), 7.38 (1H, d), 8.62 (1H, s) | 518 |
| D15 | 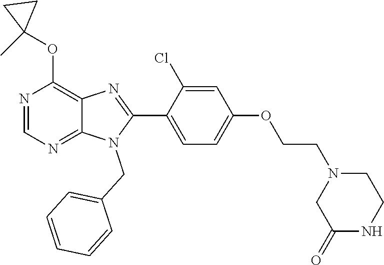 [SMILES: CC1(Oc2ncnc3c2nc(-c2ccc(OCCN4CCNC(=O)C4)cc2Cl)n3Cc2ccccc2)CC1] | 4-(2-(4- (9-benzyl- 6-(1- methylcyclo- propoxy)- 9H-purin- 8-yl)-3- chloro- phenoxy) ethyl) piperazin- 2-one | (400 MHz, DMSO-d₆): 0.78- 0.89 (2H, m), 0.97-1.07 (2H, m), 1.73 (3H, s), 2.65-2.73 (2H, m), 2.80 (2H, t), 3.06 (2H, s), 3.11-3.21 (2H, m), 4.22 (2H, t), 5.29 (2H, s), 6.87- 6.97 (2H, m), 7.04 (1H, dd), 7.17-7.24 (3H, m), 7.26 (1H, d), 7.41 (1H, d), 7.75 (1H, s), 8.61 (1H, s) | 533 |
| D16 | 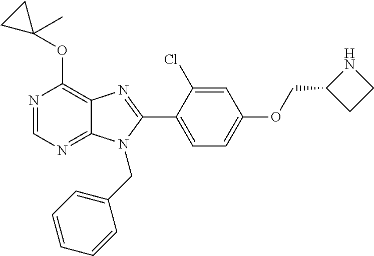 [SMILES: CC1(Oc2ncnc3c2nc(-c2ccc(OC[C@H]4CCN4)cc2Cl)n3Cc2ccccc2)CC1] | (R)-8-(4- (azetidin- 2-yl- methoxy)- 2-chloro- phenyl)-9- benzyl-6-(1- methylcyclo- propoxy)- 9H-purine | (400 MHz, DMSO-d₆): 0.78- 0.89 (2H, m), 1.01 (2H, d), 1.73 (3H, s), 2.09 (1H, p), 2.21- 2.34 (1H, m), 3.19-3.30 (1H, m), 3.51 (1H, q), 3.99- 4.16 (3H, m), 5.29 (2H, s), 6.91 (2H, dt), 7.03 (1H, dd), 7.18- 7.27 (4H, m), 7.40 (1H, d), 8.61 (1H, s). One proton not observed. | 476 |
| D17 | 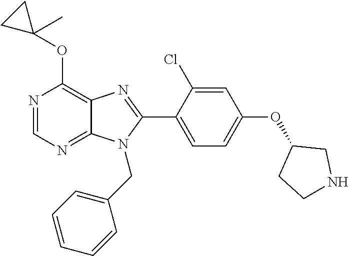 [SMILES: CC1(Oc2ncnc3c2nc(-c2ccc(O[C@H]4CCNC4)cc2Cl)n3Cc2ccccc2)CC1] | (S)-9- benzyl-8-(2- chloro-4- (pyrrolidin- 3-yloxy) phenyl)- 6-(1- methylcyclo- propoxy)- 9H-purine | (400 MHz, DMSO-d₆): 0.81- 0.88 (2H, m), 1.01 (2H, d), 1.73 (3H, s), 2.09 (1H, s), 2.20 (1H, s), 3.25 (3H, m), 3.39 (2H, m), 5.22 (1H, m), 5.29 (2H, s), 6.90 (2H, dd), 7.05 (1H, dd), 7.16-7.22 (3H, m), 7.25 (1H, d), 7.45 (1H, d), 8.62 (1H, s). | 476 |
| D18 | 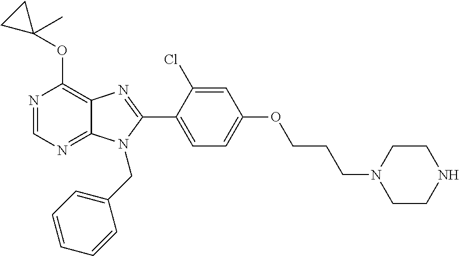 [SMILES: CC1(Oc2ncnc3c2nc(-c2ccc(OCCCN4CCNCC4)cc2Cl)n3Cc2ccccc2)CC1] | 9-benzyl- 8-(2- chloro-4-(3- (piperazin-1- yl)propoxy) phenyl)-6-(1- methylcyclo- propoxy)- 9H-purine | (400 MHz, DMSO-d₆): 0.85 (2H, d), 1.01 (2H, d), 1.73 (3H, s), 1.88 (2H, t), 2.29 (4H, s), 2.38 (2H, t), 2.68 (4H, t), 4.11 (2H, t), 5.29 (2H, s), 6.90 (2H, dd), 7.02 (1H, dd), 7.15- 7.25 (4H, m), 7.40 (1H, d), 8.61 (1H, s). One proton not observed. | 533 |
| D19 | 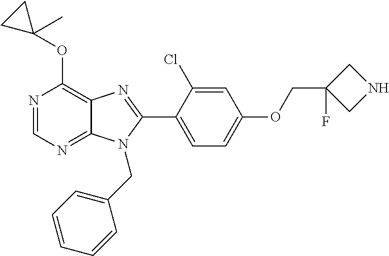 [SMILES: CC1(Oc2ncnc3c2nc(-c2ccc(OCC4(F)CNC4)cc2Cl)n3Cc2ccccc2)CC1] | 9-benzyl- 8-(2- chloro-4-((3- fluoro- azetidin-3- yl)methoxy) phenyl)-6-(1- methylcyclo- propoxy)- 9H-purine | (400 MHz, DMSO-d₆): 0.77- 0.89 (2H, m), 1.02 (2H, d), 1.73 (3H, s), 3.51 (2H, dd), 3.67 (2H, dd), 4.46 (2H, d), 5.30 (2H, s), 6.91 (2H, dd), 7.10 (1H, dd), 7.20 (3H, p), 7.33 (1H, d), 7.44 (1H, d), 8.62 (1H, s). One proton not observed. | 494 |
| D20 | 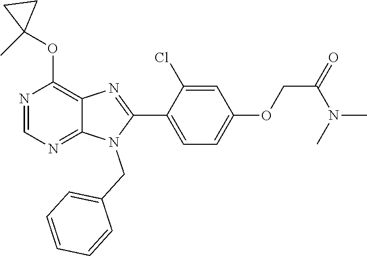 | 2-(4-(9- benzyl-6- (1- methylcyclo- propoxy)-9H- purin-8-yl)- 3-chloro- phenoxy)- N,N- dimethyl- acetamide | (400 MHz, DMSO-d₆): 0.85 (2H, d), 1.02 (2H, d), 1.73 (3H, s), 2.86 (3H, d), 3.00 (3H, d), 4.98 (2H, s), 5.29 (2H, s), 6.89 (2H, d), 7.01 (1H, dd), 7.15-7.27 (4H, m), 7.41 (1H, dd), 8.61 (1H, d). | 492 |
| D21 | 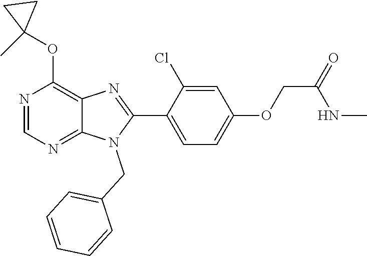 | 2-(4-(9- benzyl-6- (1- methylcyclo- propoxy)- 9H-purin- 8-yl)- 3-chloro- phenoxy)- N-methyl- acetamide | (400 MHz, DMSO-d₆): 0.85 (2H, d), 1.01 (2H, d), 1.73 (3H, s), 2.68 (3H, d), 4.60 (2H, s), 5.29 (2H, s), 6.90 (2H, dd), 7.06 (1H, dd), 7.15- 7.30 (4H, m), 7.45 (1H, d), 8.11 (1H, d), 8.62 (1H, s). | 478 |
| D22 | 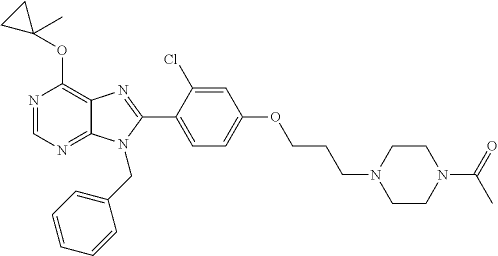 [SMILES: CC(=O)N1CCN(CCCOc2ccc(-c3nc4c(OC5(C)CC5)ncnc4n3Cc3ccccc3)c(Cl)c2)CC1] | 1-(4-(3-(4-(9- benzyl-6-(1- methylcyclo- propoxy)- 9H-purin- 8-yl)-3- chloro- phenoxy) propyl) piperazin-1- yl)ethan- 1-one | (300 MHz, DMSO-d₆): 0.71- 0.78 (2H, m), 1.08 (2H, d), 1.72 (3H, d), 1.95 (2H, s), 2.03 (3H, s), 2.45 (6H, d), 3.43 (2H, s), 3.57 (2H, s), 4.02 (2H, t), 5.27 (2H, s), 6.73 (1H, dd), 6.86 (2H, dd), 6.97 (1H, d), 7.00-7.20 (4H, m), 8.59 (1H, s). | 575 |
| D23 | 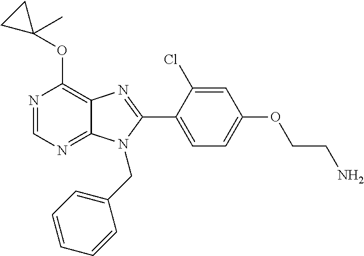 [SMILES: CC1(Oc2ncnc3c2nc(-c2ccc(OCCN)cc2Cl)n3Cc2ccccc2)CC1] | 2-(4-(9- benzyl-6- (1- methylcyclo- propoxy)- 9H-purin- 8-yl)-3- chloro- phenoxy) ethan-1- amine | (400 MHz, DMSO-d₆): 0.80- 0.88 (2H, m), 0.98-1.06 (2H, m), 1.73 (5H, s), 2.89 (2H, t), 4.02 (2H, t), 5.29 (2H, s), 6.87-6.95 (2H, m), 7.04 (1H, dd), 7.16-7.26 (4H, m), 7.41 (1H, d), 8.62 (1H, s). | 450 |
| D24 | 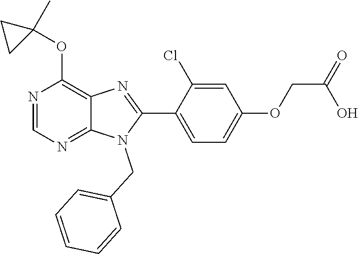 [SMILES: CC1(Oc2ncnc3c2nc(-c2ccc(OCC(=O)O)cc2Cl)n3Cc2ccccc2)CC1] | 2-(4-(9- benzyl-6- (1- methylcyclo- propoxy)- 9H-purin- 8-yl)-3- chloro- phenoxy) acetic acid | (400 MHz, DMSO-d₆): 0.64- 0.9 (2H, m), 0.9-1.09 (2H, m), 1.73 (3H, s), 4.37 (2H, s), 5.28 (2H, s), 6.83-6.94 (3H, m), 7.06 (1H, d), 7.15-7.26 (3H, m), 7.36 (1H, d), 8.60 (1H, s). One proton not observed. | 465 |
| D25 | 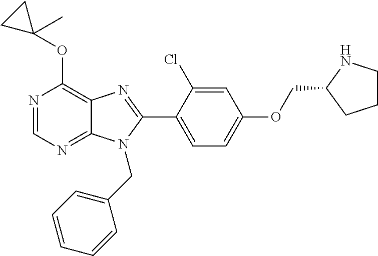 [SMILES: CC1(Oc2ncnc3c2nc(-c2ccc(OC[C@H]4CCCN4)cc2Cl)n3Cc2ccccc2)CC1] | (R)-9- benzyl-8-(2- chloro-4- (pyrrolidin-2- ylmethoxy) phenyl)-6-(1- methylcyclo- propoxy)- 9H-purine | (400 MHz, DMSO-d₆): 0.78- 0.90 (2H, m), 1.01 (2H, d), 1.37-1.51 (1H, m), 1.59-1.76 (5H, m), 1.85 (1H, m), 2.81 (2H, m), 3.41 (1H, m), 3.88- 3.93 (2H, m), 5.29 (2H, s), 6.90 (2H, dd), 7.02 (1H, dd), 7.21 (4H, m), 7.40 (1H, d), 8.61 (1H, s). One proton not observed. | 490 |
| D26 | 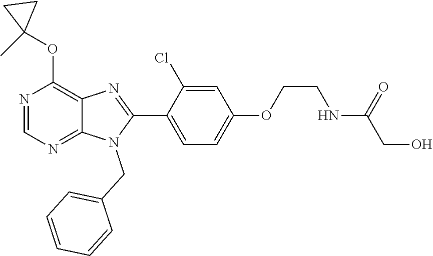 [SMILES: CC1(Oc2ncnc3c2nc(-c2ccc(OCCNC(=O)CO)cc2Cl)n3Cc2ccccc2)CC1] | N-(2-(4- (9-benzyl- 6-(1- methylcyclo- propoxy)- 9H-purin- 8-yl)-3- chloro- phenoxy) ethyl)-2- hydroxy- acetamide | (400 MHz, DMSO-d₆): 0.78- 0.89 (2H, m), 1.01 (2H, d), 1.73 (3H, s), 3.51 (2H, q), 3.83 (2H, d), 4.14 (2H, t), 5.29 (2H, s), 5.55 (1H, t), 6.90 (2H, dd), 7.04 (1H, dd), 7.20 (3H, p), 7.26 (1H, d), 7.42 (1H, d), 7.96 (1H, t), 8.62 (1H, s). | 508 |
| D27 | 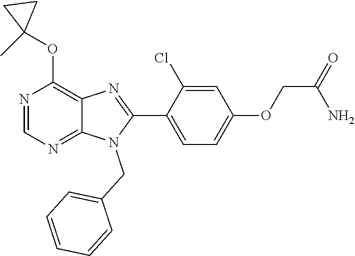 [SMILES: CC1(Oc2ncnc3c2nc(-c2ccc(OCC(N)=O)cc2Cl)n3Cc2ccccc2)CC1] | 2-(4-(9- benzyl-6- (1-methyl- cyclo- propoxy)- 9H- purin-8-yl)- 3- chloro- phenoxy) acetamide | (400 MHz, DMSO-d₆): 0.85 (2H, d), 1.02 (2H, s), 1.73 (3H, s), 4.57 (2H, s), 5.28 (2H, s), 6.86-6.93 (2H, m), 7.05 (1H, dd), 7.16-7.27 (4H, m), 7.41-7.49 (2H, m), 7.63 (1H, s), 8.62 (1H, s). | 464 |
| D28 | 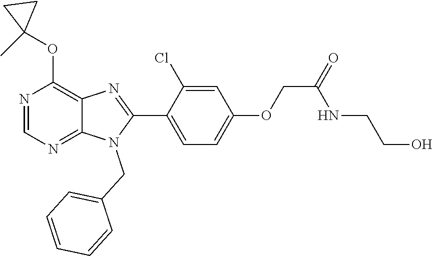 [SMILES: CC1(Oc2ncnc3c2nc(-c2ccc(OCC(=O)NCCO)cc2Cl)n3Cc2ccccc2)CC1] | 2-(4-(9- benzyl-6- (1- methylcyclo- propoxy)- 9H-purin- 8-yl)- 3-chloro- phenoxy)- N-(2- hydroxy- ethyl) acetamide | (400 MHz, DMSO-d₆): 0.80- 0.88 (2H, m), 1.01 (2H, d), 1.73 (3H, s), 3.23 (2H, q), 3.45 (2H, q), 4.62 (2H, s), 4.75 (1H, t), 5.29 (2H, s), 6.90 (2H, dt), 7.06 (1H, dd), 7.21 (3H, dq), 7.27 (1H, d), 7.45 (1H, d), 8.14 (1H, t), 8.62 (1H, s). | 508 |
| D29 | 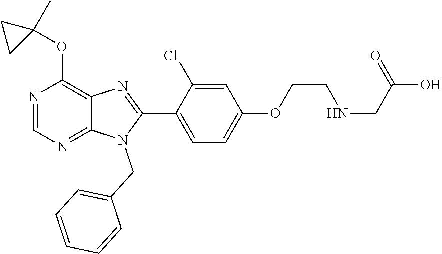 [SMILES: CC1(Oc2ncnc3c2nc(-c2ccc(OCCNCC(=O)O)cc2Cl)n3Cc2ccccc2)CC1] | (2-(4-(9- benzyl-6- (1- methylcyclo- propoxy)- 9H-purin- 8-yl)-3- chloro- phenoxy) ethyl) glycine | (400 MHz, DMSO-d₆): 0.84 (2H, s), 1.02 (2H, s), 1.73 (3H, s), 3.19 (2H, s), 3.36 (2H, s), 4.27 (2H, t), 5.29 (2H, s), 6.87-6.94 (2H, m), 7.06 (1H, dd), 7.17-7.23 (3H, m), 7.26 (1H, d), 7.45 (1H, d), 8.62 (1H, s). Two protons not observed. | 508 |
| D30 | 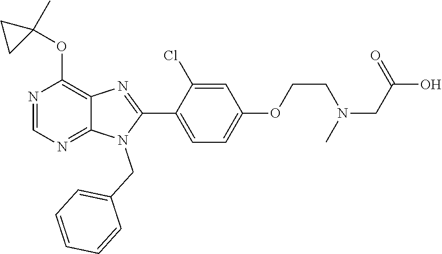 [SMILES: CN(CCOc1ccc(-c2nc3c(OC4(C)CC4)ncnc3n2Cc2ccccc2)c(Cl)c1)CC(=O)O] | N-(2-(4- (9-benzyl- 6-(1- methylcyclo- propoxy)- 9H-purin- 8-yl)-3- chloro- phenoxy) ethyl)-N- methyl- glycine | (300 MHz, DMSO-d₆): 0.72- 0.94 (2H, m), 1-1.13 (2H, m), 1.73 (3H, s), 2.45 (3H, s), 3.00 (2H, t), 3.32 (2H,s), 4.20 (2H, t), 5.29 (2H, s), 6.77-6.98 (2H, m), 6.98-7.13 (1H, m), 7.13-7.3 (4H, m), 7.41 (1H, d), 8.62 (1H, s). One proton not observed. | 522 |
| D31 | 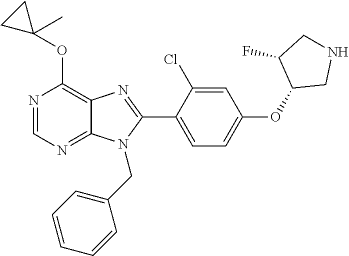 [SMILES: CC1(Oc2ncnc3c2nc(-c2ccc(O[C@H]4CNC[C@H]4F)cc2Cl)n3Cc2ccccc2)CC1] | 9-benzyl- 8-(2- chloro-4- (((3S,4R)- 4-fluoro- pyrrolidin- 3-yl)oxy) phenyl)- 6-(1- methylcyclo- propoxy)- 9H-purine | (400 MHz, DMSO-d₆): 0.78- 0.87 (2H, m), 0.99-1.06 (2H, m), 1.73 (3H, s), 2.85 (1H, d), 2.98 (1H, dd), 3.17 (2H, s), 4.88 (1H, m), 5.19- 5.38 (3H, m), 6.87-6.93 (2H, m), 7.12 (1H, dd), 7.17- 7.23 (3H, m), 7.33 (1H, d), 7.41 (1H, d), 8.62 (1H, s). One proton not observed. | 494 |
| D32 | 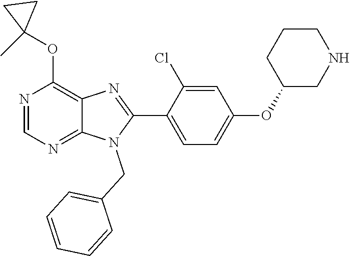 [SMILES: CC1(Oc2ncnc3c2nc(-c2ccc(O[C@@H]4CCCNC4)cc2Cl)n3Cc2ccccc2)CC1] | (R)-9- benzyl- 8-(2- chloro-4- (piperidin-3- yloxy) phenyl)-6- (1- methylcyclo- propoxy)- 9H-purine | (400 MHz, DMSO-d₆): 0.84 (2H, t), 1.01 (2H, t), 1.38-1.59 (2H, m), 1.64-1.69 (1H, m), 1.73 (3H, s), 2.02-2.06 (1H, m), 2.43-2.47 (1H, m), 2.50-2.56 (1H, m), 2.68-2.83 (1H, m), 3.06-3.13 (1H, m), 4.26-4.44 (1H, m), 5.29 (2H, s), 6.83-6.95 (2H, m), 7.02-7.04 (1H, m), 7.13-7.26 (4H, m), 7.38 (1H, d), 8.61 (1H, s). One proton not observed. | 490 |
| D33 | 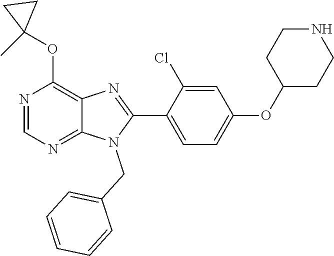 [SMILES: CC1(Oc2ncnc3c2nc(-c2ccc(OC4CCNCC4)cc2Cl)n3Cc2ccccc2)CC1] | 9-benzyl- 8-(2- chloro-4- (piperidin-4- yloxy) phenyl)-6- (1- methylcyclo- propoxy)- 9H-purine | (400 MHz, DMSO-d₆): 0.80- 0.87 (2H, m), 0.97-1.05 (2H, m), 1.38-1.53 (2H, m), 1.73 (3H, s), 1.92-1.96 (2H, m), 2.56-5.62 (2H, m), 2.95 (2H, m), 4.56 (1H, m), 5.29 (2H, s), 6.83-6.92 (2H, m), 7.02-7.05 (1H, m), 7.16-7.26 (4H, m), 7.37 (1H, d), 8.61 (1H, s). One proton not observed. | 490 |
| D34 | 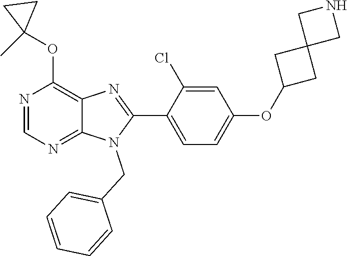 [SMILES: CC1(Oc2ncnc3c2nc(-c2ccc(OC4CC5(CNC5)C4)cc2Cl)n3Cc2ccccc2)CC1] | 8-(4-((2- azaspiro [3.3] heptan-6- yl)oxy)- 2-chloro- phenyl)-9- benzyl-6-(1- methylcyclo- propoxy)- 9H-purine | (400 MHz, DMSO-d₆): 0.85 (2H, t), 1.01 (2H, t), 1.73 (3H, s), 2.09-2.22 (2H, m), 2.67- 2.76 (2H, m), 3.44-3.56 (3H, m), 3.71-3.9 (2H, m), 4.68- 4.74 (1H, m), 5.26 (2H, s), 6.86-6.93 (3H, m), 7.10 (1H, d), 7.16-7.23 (3H, m), 7.38 (1H, d), 8.61 (1H, s). | 502 |
| D35 | 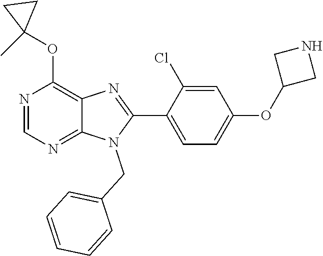 [SMILES: CC1(Oc2ncnc3c2nc(-c2ccc(OC4CNC4)cc2Cl)n3Cc2ccccc2)CC1] | 8-(4- (azetidin-3- yloxy)-2- chloro- phenyl)-9- benzyl-6-(1- methylcyclo- propoxy)- 9H-purine | (400 MHz, DMSO-d₆): 0.84 (2H, t), 1.01 (2H, t), 1.73 (3H, s), 3.52 (2H, t), 3.81 (2H, t), 5.05-5.15 (1H, m), 5.28 (2H, s), 6.75-6.95 (3H, m), 7.07 (1H, d), 7.11-7.26 (3H, m), 7.40 (1H, d), 8.61 (1H, s). | 462 |

[D1] was obtained from 1-(4-(hydroxymethyl)piperidin-1-yl)ethan-1-one and 4-(9-benzyl-6-(1-methylcyclopropoxy)-9H-purin-8-yl)-3-chlorophenol (Synthetic Example 5) using a Mitsunobu reaction conducted at rt for 18 hours using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5).

[D2] was obtained from 2-(1-methylpyrrolidin-2-yl)ethan-1-ol and 4-(9-benzyl-6-(1-methylcyclopropoxy)-9H-purin-8-yl)-3-chlorophenol (Synthetic Example 5) using a Mitsunobu reaction conducted at rt for 18 hours using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5).

[D3]9-benzyl-8-(2-chloro-4-(2-(1-methylpyrrolidin-2-yl)ethoxy)phenyl)-6-(1-methylcyclopropoxy)-9H-purine (Isomer 2), Synthetic Example D3 and 9-benzyl-8-(2-chloro-4-(2-(1-methylpyrrolidin-2-yl)ethoxy)phenyl)-6-(1-methylcyclopropoxy)-9H-purine (Isomer 1), Synthetic Example D4 were obtained from racemic 9-benzyl-8-(2-chloro-4-(2-(1-methylpyrrolidin-2-yl)ethoxy)phenyl)-6-(1-methylcyclopropoxy)-9H-purine [Synthetic Example D2] by separation of the two enantiomers by chiral SFC (Phenomenex Lux column iC5, 21.2*250 mm, 5 micron) eluting with 40% MeOH/MeCN (1:1; containing 0.1% NH₃) and 60% scCO₂ at a flow rate of 60 mL/min, BRP of 120 bar and column temperature of 40° C.; Synthetic Example D4 eluting first and Synthetic Example D3 eluting second in the purification

[D5] was obtained from 1-(4-(2-hydroxyethyl)piperazin-1-yl)ethan-1-one and 4-(9-benzyl-6-(1-methylcyclopropoxy)-9H-purin-8-yl)-3-chlorophenol (see Synthetic Example 5) using a Mitsunobu reaction conducted at rt for 21 hours using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5).

[D6] was obtained from tert-butyl (3-hydroxypropyl)carbamate and 4-(9-benzyl-6-(1-methylcyclopropoxy)-9H-purin-8-yl)-3-chlorophenol (see Synthetic Example 5) using a Mitsunobu reaction conducted at rt for 18 hours using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5). The BOC group of tert-butyl (3-(4-(9-benzyl-6-(1-methylcyclopropoxy)-9H-purin-8-yl)-3-chlorophenoxy)propyl)carbamate was then removed by reaction of HCl in MeOH.

Tert-butyl (3-(4-(9-benzyl-6-(1-methylcyclopropoxy)-9H-purin-8-yl)-3-chlorophenoxy)propyl)carbamate. m/z: ES+[M+H]+ 464.

[D7] was obtained from 3-(4-(9-benzyl-6-(1-methylcyclopropoxy)-9H-purin-8-yl)-3-chlorophenoxy)propan-1-amine (Synthetic Example D6) via an acylation using acetic anhydride and triethylamine.

[D8] was obtained from 1-(4-(2-hydroxyethyl)piperazin-1-yl)ethan-1-one and 3-(9-benzyl-6-(1-methylcyclopropoxy)-9H-purin-8-yl)-2-chlorophenol using a Mitsunobu reaction conducted at rt for 5 hours using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5).

3-(9-Benzyl-6-(1-methylcyclopropoxy)-9H-purin-8-yl)-2-chlorophenol was made from 2-chloro-3-hydroxybenzaldehyde and N-benzyl-6-(1-methylcyclopropoxy)-5-nitropyrimidin-4-amine (Synthetic Example 4, intermediate) using a similar method previously described in the synthesis of 2-(2-chloro-4-(2-(4-methylpiperazin-1-yl)ethoxy)phenyl)-1-(3-chlorobenzyl)-5-methoxy-1H-benzo[d]imidazole (Synthetic Example 2, intermediate). ¹H NMR (300 MHz, CD₃OD): 0.71-0.79 (2H, m), 0.96-1.04 (2H, m), 1.69 (3H, s), 5.25 (2H, s), 6.65 (1H, dd), 6.80 (2H, dd), 6.99-7.13 (5H, m), 8.51 (1H, s). One proton not observed. m/z: ES+[M+H]+ 407.

[D9] was obtained from 1-methylpiperidin-4-ol and 3-(9-benzyl-6-(1-methylcyclopropoxy)-9H-purin-8-yl)-2-chlorophenol (see Synthetic Example D8, intermediate) using a Mitsunobu reaction conducted at 0° C. for 5 hours using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5).

[D10] was obtained from tert-butyl 4-(2-hydroxyethyl)piperazine-1-carboxylate and 3-(9-benzyl-6-(1-methylcyclopropoxy)-9H-purin-8-yl)-2-chlorophenol (see Synthetic Example D8, intermediate) using a Mitsunobu reaction conducted at rt for 16 hours using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5). The BOC group of tert-butyl 4-(2-(3-(9-benzyl-6-(1-methylcyclopropoxy)-9H-purin-8-yl)-2-chlorophenoxy)ethyl)piperazine-1-carboxylate was then removed under HCl in MeOH.

Tert-butyl 4-(2-(3-(9-benzyl-6-(1-methylcyclopropoxy)-9H-purin-8-yl)-2-chlorophenoxy)ethyl)piperazine-1-carboxylate. m/z: ES+[M+H]+ 619.

[D 11] was obtained from tert-butyl 4-hydroxypiperidine-1-carboxylate and 3-(9-benzyl-6-(1-methylcyclopropoxy)-9H-purin-8-yl)-2-chlorophenol (see Synthetic Example D8, intermediate) using a Mitsunobu reaction conducted at rt for 16 hours using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5). The BOC group of tert-butyl 4-(3-(9-benzyl-6-(1-methylcyclopropoxy)-9H-purin-8-yl)-2-chlorophenoxy)piperidine-1-carboxylate was then removed under HCl in MeOH.

Tert-butyl 4-(3-(9-benzyl-6-(1-methylcyclopropoxy)-9H-purin-8-yl)-2-chlorophenoxy)piperidine-1-carboxylate m/z: ES+[M+H]+ 590.

[D12] was obtained from 2-(4-methylpiperazin-1-yl)ethan-1-ol and 3-(9-benzyl-6-(1-methylcyclopropoxy)-9H-purin-8-yl)-2-chlorophenol (see Synthetic Example D8, intermediate) using a Mitsunobu reaction conducted at rt for 3 hours using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5).

[D13]9-Benzyl-8-(2-chloro-4-((1-methylazepan-4-yl)oxy)phenyl)-6-(1-methylcyclopropoxy)-9H-purine (Isomer 1), Synthetic Example D13 and 9-benzyl-8-(2-chloro-4-((1-methylazepan-4-yl)oxy)phenyl)-6-(1-methylcyclopropoxy)-9H-purine (Isomer 2), Synthetic Example D14 were obtained from the racemic 9-benzyl-8-(2-chloro-4-((1-methylazepan-4-yl)oxy)phenyl)-6-(1-methylcyclopropoxy)-9H-purine, which was prepared using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5), except the racemate was purified by flash C18-flash chromatography, elution gradient 0 to 100% MeCN in water (0.5% formic acid). The enantiomers were separated by chiral HPLC (CHIRALPAK AD-H column, 2*25 cm, 5 μm eluting with a gradient of EtOH in hexane (containing 0.5% 2M NH₃ in MeOH) to afford the first eluting enantiomer [Synthetic Example D13] and the second eluting enantiomer [Synthetic Example D14]. The separated enantiomers were further purified by preparative HPLC (XBridge Prep OBD C18 column, 30*150 mm, 5 μm), using decreasingly polar mixtures of water (containing 10 mmol/L NH₄HCO₃ and 0.1% aqueous ammonia) and MeCN as eluents.

[D15] was obtained from 2-chloro-4-(2-(3-oxopiperazin-1-yl)ethoxy)benzaldehyde and N4-benzyl-6-(1-methylcyclopropoxy)pyrimidine-4,5-diamine (Synthetic Example 4, intermediate) by a cyclisation reaction similar to the previously described procedure to form 9-benzyl-8-(2-chloro-4-(2-(4-methylpiperazin-1-yl)ethoxy)phenyl)-6-(1-methylcyclopropoxy)-9H-purine (Synthetic Example 4).

2-Chloro-4-(2-(3-oxopiperazin-1-yl)ethoxy)benzaldehyde was made by N-alkylation using a similar procedure used in the synthesis of (S)-9-benzyl-8-(2-chloro-4-(2-(3-methylpiperazin-1-yl)ethoxy)phenyl)-6-(1-methylcyclopropoxy)-9H-purine (Synthetic Example R3), using piperazin-2-one hydrochloride and 4-(2-bromoethoxy)-2-chlorobenzaldehyde (Synthetic Example 18, intermediate) as starting material. ¹H NMR (300 MHz, DMSO-d₆): 2.64-2.71 (2H, m), 2.80 (2H, t), 3.05 (2H, s), 3.11-3.19 (2H, m), 4.26 (2H, t), 7.11 (1H, dd), 7.24 (1H, d), 7.75 (1H, s), 7.83 (1H, d), 10.20 (1H, s). m/z: ES+[M+H]+ 283.

[D16] was obtained from tert-butyl (R)-2-(hydroxymethyl)azetidine-1-carboxylate and 4-(9-benzyl-6-(1-methylcyclopropoxy)-9H-purin-8-yl)-3-chlorophenol (Synthetic Example 5, intermediate), using a Mitsunobu reaction conducted at rt for 4 hours using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5). The BOC group of tert-butyl (R)-2-((4-(9-benzyl-6-(1-methylcyclopropoxy)-9H-purin-8-yl)-3-chlorophenoxy)methyl)azetidine-1-carboxylate was then removed under HCl in dioxane.

Tert-butyl (R)-2-((4-(9-benzyl-6-(1-methylcyclopropoxy)-9H-purin-8-yl)-3-chlorophenoxy)methyl)azetidine-1-carboxylate. ¹H NMR (300 MHz, DMSO-d₆): 0.79-0.87 (2H, m), 1.01 (2H, d), 1.34 (9H, s), 1.73 (3H, s), 2.15 (1H, q), 2.33 (1H, t), 3.78 (2H, t), 4.18 (1H, dd), 4.32-4.53 (2H, m), 5.29 (2H, s), 6.91 (2H, dd), 7.08 (1H, dd), 7.20 (3H, qd), 7.28 (1H, d), 7.42 (1H, d), 8.62 (1H, s). m/z: ES+[M+H]+ 576.

[D17] was obtained from tert-butyl (R)-3-hydroxypyrrolidine-1-carboxylate and 4-(9-benzyl-6-(1-methylcyclopropoxy)-9H-purin-8-yl)-3-chlorophenol (Synthetic Example 5, intermediate), using a Mitsunobu reaction conducted at rt for 1 hour using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5).

The BOC group of tert-butyl (S)-3-(4-(9-benzyl-6-(1-methylcyclopropoxy)-9H-purin-8-yl)-3-chlorophenoxy)pyrrolidine-1-carboxylate was then removed under HCl in dioxane.

Tert-butyl (S)-3-(4-(9-benzyl-6-(1-methylcyclopropoxy)-9H-purin-8-yl)-3-chlorophenoxy)pyrrolidine-1-carboxylate, ¹H NMR (300 MHz, DMSO-d₆): 0.80-0.87 (m, 2H), 1.01 (d, 2H), 1.41 (d, 9H), 1.73 (s, 3H), 2.13 (d, 2H), 3.35-3.62 (m, 4H), 5.15 (s, 1H), 5.29 (s, 2H), 6.89 (dd, 2H), 7.04 (dd, 1H), 7.15-7.27 (m, 4H), 7.40 (d, 1H), 8.62 (s, 1H). m/z: ES+[M+H]+ 576.

[D18] was obtained from tert-butyl 4-(3-hydroxypropyl)piperazine-1-carboxylate and 4-(9-benzyl-6-(1-methylcyclopropoxy)-9H-purin-8-yl)-3-chlorophenol (Synthetic Example 5, intermediate), using a Mitsunobu reaction conducted at rt for 1 hour using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5).

The BOC group of tert-butyl 4-(3-(4-(9-benzyl-6-(1-methylcyclopropoxy)-9H-purin-8-yl)-3-chlorophenoxy)propyl)piperazine-1-carboxylate was then removed under HCl in dioxane. Tert-butyl 4-(3-(4-(9-benzyl-6-(1-methylcyclopropoxy)-9H-purin-8-yl)-3-chlorophenoxy)propyl)piperazine-1-carboxylate. ¹H NMR (300 MHz, DMSO-d₆): 0.85 (2H, d), 1.01 (2H, d), 1.39 (9H, s), 1.73 (3H, s), 1.90 (2H, dd), 2.33 (4H, t), 2.44 (2H, t), 3.26-3.32 (4H, m), 4.12 (2H, t), 5.29 (2H, s), 6.86-6.95 (2H, m), 7.02 (1H, dd), 7.20 (4H, ddd), 7.40 (1H, d), 8.61 (1H, s). m/z: ES+[M+H]+ 633.

[D19] was obtained from tert-butyl 3-fluoro-3-(hydroxymethyl)azetidine-1-carboxylate and 4-(9-benzyl-6-(1-methylcyclopropoxy)-9H-purin-8-yl)-3-chlorophenol (Synthetic Example 5, intermediate), using a Mitsunobu reaction conducted at rt for 4 hours using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5).

The BOC group of tert-butyl 3-((4-(9-benzyl-6-(1-methylcyclopropoxy)-9H-purin-8-yl)-3-chlorophenoxy)methyl)-3-fluoroazetidine-1-carboxylate was then removed under HCl in dioxane.

Tert-butyl 3-((4-(9-benzyl-6-(1-methylcyclopropoxy)-9H-purin-8-yl)-3-chlorophenoxy)methyl)-3-fluoroazetidine-1-carboxylate. ¹H NMR (300 MHz, DMSO-d₆): 0.80-0.88 (2H, m), 1.01 (2H, d), 1.40 (9H, s), 1.73 (3H, s), 3.99 (1H, s), 4.06-4.18 (3H, m), 4.47 (1H, s), 4.55 (1H, s), 5.29 (2H, s), 6.91 (2H, dd), 7.08 (1H, dd), 7.18-7.23 (3H, m), 7.30 (1H, d), 7.45 (1H, d), 8.62 (1H, s). m/z: ES+[M+H]+ 594.

[D20] was obtained from 2-hydroxy-N-methylacetamide and 4-(9-benzyl-6-(1-methylcyclopropoxy)-9H-purin-8-yl)-3-chlorophenol (Synthetic Example 5, intermediate), using a Mitsunobu reaction conducted at rt for 1 hour using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5).

[D21] was obtained from 2-hydroxy-N,N-dimethylacetamide and 4-(9-benzyl-6-(1-methylcyclopropoxy)-9H-purin-8-yl)-3-chlorophenol (Synthetic Example 5, intermediate), using a Mitsunobu reaction conducted at rt for 1 hour using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5).

[D22] was obtained from 9-benzyl-8-(2-chloro-4-(3-(piperazin-1-yl)propoxy)phenyl)-6-(1-methylcyclopropoxy)-9H-purine (Synthetic Example D18) via acylation using acetic anhydride and triethylamine.

[D23] was obtained from tert-butyl (2-hydroxyethyl)carbamate and 4-(9-benzyl-6-(1-methylcyclopropoxy)-9H-purin-8-yl)-3-chlorophenol (Synthetic Example 5, intermediate), using a Mitsunobu reaction conducted at rt for 3 hours using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5).

The BOC group of tert-butyl (2-(4-(9-benzyl-6-(1-methylcyclopropoxy)-9H-purin-8-yl)-3-chlorophenoxy)ethyl)carbamate was then removed under HCl in dioxane.

Tert-butyl (2-(4-(9-benzyl-6-(1-methylcyclopropoxy)-9H-purin-8-yl)-3-chlorophenoxy)ethyl)carbamate, ¹H NMR (300 MHz, DMSO-d₆): 0.79-0.86 (2H, m), 1.00 (2H, d), 1.37 (9H, s), 1.71 (3H, s), 3.28 (1H, s), 3.33 (1H, d), 4.03-4.10 (2H, m), 5.27 (2H, s), 6.86-6.93 (2H, m), 7.01 (1H, dd), 7.16-7.22 (4H, m), 7.39 (1H, d), 8.60 (1H, s). One proton not observed. m/z: ES+[M+H]+ 550.

[D24] obtained from alkylation of 4-(9-benzyl-6-(1-methylcyclopropoxy)-9H-purin-8-yl)-3-chlorophenol (Synthetic Example 5, intermediate) with ethyl 2-bromoacetate, using similar conditions in the synthesis of 3-(3-chloro-4-formylphenoxy)propanoic acid (Synthetic Example H11, intermediate). This was followed by ester hydrolysis of ethyl 2-(4-(9-benzyl-6-(1-methylcyclopropoxy)-9H-purin-8-yl)-3-chlorophenoxy)acetate with sodium hydroxide.

Ethyl 2-(4-(9-benzyl-6-(1-methylcyclopropoxy)-9H-purin-8-yl)-3-chlorophenoxy)acetate, ¹H NMR (300 MHz, DMSO-d₆): 0.85 (2H, d), 1.02 (2H, s), 1.20-1.25 (3H, m), 1.73 (3H, s), 4.16-4.25 (2H, m), 4.95 (2H, s), 5.29 (2H, s), 6.89 (2H, t), 7.05 (1H, dt), 7.17-7.24 (3H, m), 7.27 (1H, t), 7.37-7.49 (1H, m), 8.62 (1H, d). m/z: ES+[M+H]+ 493.

[D25] was obtained from tert-butyl (R)-2-(hydroxymethyl)pyrrolidine-1-carboxylate and 4-(9-benzyl-6-(1-methylcyclopropoxy)-9H-purin-8-yl)-3-chlorophenol (Synthetic Example 5, intermediate), using a Mitsunobu reaction conducted at rt for 2 hours using a similar procedure used in the synthesis of 9-benzyl-8-(2-chloro-4-((1-methylpiperidin-4-yl)methoxy)phenyl)-6-(1-methylcyclopropoxy)-9H-purine (Synthetic Example 5).

The BOC group of tert-butyl (R)-2-((4-(9-benzyl-6-(1-methylcyclopropoxy)-9H-purin-8-yl)-3-chlorophenoxy)methyl)pyrrolidine-1-carboxylate was then removed under HCl in dioxane. Tert-butyl (R)-2-((4-(9-benzyl-6-(1-methylcyclopropoxy)-9H-purin-8-yl)-3-chlorophenoxy)methyl)pyrrolidine-1-carboxylate, ¹H NMR (300 MHz, DMSO-d₆): 0.80-0.87 (2H, m), 1.01 (2H, d), 1.40 (9H, d), 1.76 (6H, d), 1.94 (1H, s), 3.26 (2H, s), 4.13 (1H, t), 4.75-4.80 (2H, m), 5.29 (2H, s), 6.86-6.94 (2H, m), 7.06 (1H, dd), 7.16-7.23 (3H, m), 7.26 (1H, s), 7.41 (1H, d), 8.61 (1H, s). m/z: ES+[M+H]+ 590.

[D26] was obtained from 2-(4-(9-benzyl-6-(1-methylcyclopropoxy)-9H-purin-8-yl)-3-chlorophenoxy)ethan-1-amine (Synthetic Example D23) by amide coupling with 2-hydroxyacetic acid as follows: 2-Hydroxyacetic acid (50.7 mg, 0.67 mmol), 1-(3-dimethylaminopropyl)-3-ethylcarbodiimide hydrochloride (63.9 mg, 0.33 mmol), DIEA (194 μL, 1.11 mmol) and HOBt (51.1 mg, 0.33 mmol) in DMF (2 mL) were stirred at rt for 15 minutes. Then 2-(4-(9-benzyl-6-(1-methylcyclopropoxy)-9H-purin-8-yl)-3-chlorophenoxy)ethan-1-amine (100 mg, 0.22 mmol) was added to the mixture. The mixture was stirred at rt for 2 hours. The reaction mixture was diluted with water (25 mL), and extracted with EtOAc (3×20 mL). The organic layer was dried over Na₂SO₄, filtered and evaporated. The residue was purified by preparative TLC (DCM: MeOH=10:1, then by preparative chromatography on silica (column: 19*150 mm, 5 μm); eluant: gradient from 5% to 35% EtOH in n-hexane. Fractions containing the desired compound were evaporated to dryness to afford N-(2-(4-(9-benzyl-6-(1-methylcyclopropoxy)-9H-purin-8-yl)-3-chlorophenoxy)ethyl)-2-hydroxyacetamide (19 mg, 17%) as a white solid.

[D27] was obtained from 2-(4-(9-benzyl-6-(1-methylcyclopropoxy)-9H-purin-8-yl)-3-chlorophenoxy)acetic acid (Synthetic Example D24) by amide coupling with ammonia as follows:

2-(4-(9-Benzyl-6-(1-methylcyclopropoxy)-9H-purin-8-yl)-3-chlorophenoxy)acetic acid (130 mg, 0.28 mmol), HATU (159 mg, 0.42 mmol) and DIEA (0.195 mL, 1.12 mmol) in DMA (3 mL) was stirred at rt for 15 minutes. Then conc. aq. ammonia (1.3 mL, 0.28 mmol) was added to the mixture and stirred at rt for 2 hours. The reaction mixture was concentrated and diluted with water (25 mL), and extracted with EtOAc (3×20 mL). The organic layer was dried over Na₂SO₄, filtered and evaporated to afford crude product. The crude product was purified by preparative HPLC (YMC-Actus Triart C18 ExRS column, 30*150 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness to afford 2-(4-(9-benzyl-6-(1-methylcyclopropoxy)-9H-purin-8-yl)-3-chlorophenoxy)acetamide (26 mg, 20%) as a white solid.

[D28] was obtained from 2-(4-(9-benzyl-6-(1-methylcyclopropoxy)-9H-purin-8-yl)-3-chlorophenoxy)acetic acid (Synthetic Example D24) by amide coupling with 2-aminoethan-1-ol following a similar procedure to the synthesis of 2-(4-(9-benzyl-6-(1-methylcyclopropoxy)-9H-purin-8-yl)-3-chlorophenoxy)acetamide (Synthetic Example D27).

[D29] was obtained from 2-(4-(9-benzyl-6-(1-methylcyclopropoxy)-9H-purin-8-yl)-3-chlorophenoxy)ethan-1-amine (Synthetic Example D23) as follows:

Sodium hydride (28 mg, 0.70 mmol) was added to 2-(4-(9-benzyl-6-(1-methylcyclopropoxy)-9H-purin-8-yl)-3-chlorophenoxy)ethan-1-amine (210 mg, 0.47 mmol) in DMF (2 mL) at 0° C. The resulting mixture was stirred at rt for 10 minutes. Then ethyl 2-bromoacetate (78 mg, 0.47 mmol) was added to the mixture. The resulting mixture was stirred at rt for 1 hour. Then more sodium hydride (18.7 mg, 0.47 mmol) was added. The mixture was stirred at rt for 10 minutes and then Mel (14.59 μL, 0.23 mmol) was added to the mixture. The resulting mixture was stirred at rt for 1 hour. The reaction mixture was quenched with water (0.1 mL). (2-(4-(9-Benzyl-6-(1-methylcyclopropoxy)-9H-purin-8-yl)-3-chlorophenoxy)ethyl)glycine was isolated as a by-product by purification by preparative HPLC (XSelect CSH Fluoro Phenyl column, 30*150 mm, 5 μm), using decreasingly polar mixtures of water (containing 0.1% formic acid) and MeCN as eluents. Fractions containing the corresponding compound were evaporated to dryness to afford (2-(4-(9-benzyl-6-(1-methylcyclopropoxy)-9H-purin-8-yl)-3-chlorophenoxy)ethyl)glycine (32 mg, 13%) as a pink solid.

[D30] was obtained from the cyclisation of (E)-N-(2-(4-(((4-(benzylamino)-6-(1-methylcyclopropoxy)pyrimidin-5-yl)imino)methyl)-3-chlorophenoxy)ethyl)-N-methylglycine
using a similar method to Synthetic Example 4.

(E)-N-(2-(4-(((4-(Benzylamino)-6-(1-methylcyclopropoxy)pyrimidin-5-yl)imino)methyl)-3-chlorophenoxy)ethyl)-N-methylglycine was made from reaction of N4-benzyl-6-(1-methylcyclopropoxy)pyrimidine-4,5-diamine (Synthetic Example 4, intermediate) and N-(2-(3-chloro-4-formylphenoxy)ethyl)-N-methylglycine using a similar method to Synthetic Example 4. m/z: ES+[M+H]+ 524.

N-(2-(3-Chloro-4-formylphenoxy)ethyl)-N-methylglycine was made from the ester hydrolysis of ethyl N-(2-(3-chloro-4-formylphenoxy)ethyl)-N-methylglycinate under basic LiOH conditions. ¹H NMR (300 MHz, DMSO-d₆): 2.44 (3H, s), 3.01 (2H, t), 3.33 (2H, s), 4.24 (2H, t), 7.08 (1H, m), 7.20 (1H, d), 7.83 (1H, d), 8.15 (1H, s), 10.19 (1H, d). m/z: ES+[M+H]+ 272.

Ethyl N-(2-(3-chloro-4-formylphenoxy)ethyl)-N-methylglycinate was made from ethyl 2-bromoacetate and 2-chloro-4-(2-(methylamino)ethoxy)benzaldehyde by N-alkylation as follows:

Ethyl 2-bromoacetate (0.504 g, 3.02 mmol) was added in one portion to 2-chloro-4-(2-(methylamino)ethoxy)benzaldehyde (0.43 g, 2.01 mmol) and triethylamine (0.611 g, 6.04 mmol) in THF (20 mL) at 25° C. under air. The resulting solution was stirred at 25° C. for 16 hours. The reaction mixture was diluted with water (200 mL). The aqueous layer was extracted with EtOAc (3×50 mL). The organic layer was dried over Na₂SO₄, filtered and evaporated to afford crude product. The crude product was purified by flash silica chromatography, elution gradient 0 to 20% MeOH in DCM. Pure fractions were evaporated to dryness to afford ethyl N-(2-(3-chloro-4-formylphenoxy)ethyl)-N-methylglycinate (0.450 g, 75%) as a pale yellow oil. ¹H NMR (300 MHz, DMSO-d₆): 1.28 (3H, t), 2.53 (3H, s), 3.04 (2H, t), 3.41 (2H, s), 4.06-4.22 (4H, m), 6.77-7.04 (2H, m), 7.88 (1H, d), 10.33 (1H, t). m/z: ES+[M+H]+ 300.

2-Chloro-4-(2-(methylamino)ethoxy)benzaldehyde was made by N-alkylation of methanamine with 4-(2-bromoethoxy)-2-chlorobenzaldehyde (Synthetic Example 18, intermediate) as follows.

4-(2-Bromoethoxy)-2-chlorobenzaldehyde (540 mg, 2.05 mmol) was added in one portion to methanamine (7.97 g, 102.5 mmol) aqueous solution and THF (1 mL) was then added. The resulting solution was stirred at rt for 6 hours. The solvent was removed under reduced pressure to get crude 2-chloro-4-(2-(methylamino)ethoxy)benzaldehyde (0.43 g), which was used directly for next step. m/z: ES+[M+H]+ 214.

[D31]K₂CO₃ (143 mg, 1.03 mmol) was added to 4-(9-benzyl-6-(1-methylcyclopropoxy)-9H-purin-8-yl)-3-chlorophenol (140 mg, 0.34 mmol, Synthetic Example 5 Intermediate) and tert-butyl (3R,4R)-3-fluoro-4-((methylsulfonyl)oxy)pyrrolidine-1-carboxylate (117 mg, 0.41 mmol) in DMA (4 mL) at rt. The resulting mixture was stirred at 120° C. for 2 days. The reaction mixture was concentrated, diluted with water (25 mL) and extracted with EtOAc (25 mL*3). The combined organic layers were washed with saturated brine (25 mL), dried over Na₂SO₄, filtered and evaporated to afford crude tert-butyl (3S,4R)-3-(4-(9-benzyl-6-(1-methylcyclopropoxy)-9H-purin-8-yl)-3-chlorophenoxy)-4-fluoropyrrolidine-1-carboxylate (150 mg, 73%). The product was used in the next step directly without further purification.

m/z: ES+[M+H]+ 594

HCl in dioxane (0.5 ml, 0.50 mmol) was added to tert-butyl (3S,4R)-3-(4-(9-benzyl-6-(1-methylcyclopropoxy)-9H-purin-8-yl)-3-chlorophenoxy)-4-fluoropyrrolidine-1-carboxylate (130 mg, 0.22 mmol) in EtOAc (1 mL) at rt. The resulting mixture was stirred at RT for 2 hours. The solvent was removed under reduced pressure. The crude product was purified by preparative HPLC Column: XSelect CSH Fluoro Phenyl, 30*150 mm, 5 m; using decreasingly polar mixtures of acetonitrile in water (0.1% formic acid). Fractions containing the desired compound were evaporated to dryness to afford 9-benzyl-8-(2-chloro-4-(((3S,4R)-4-fluoropyrrolidin-3-yl)oxy)phenyl)-6-(1-methylcyclopropoxy)-9H-purine (13 mg, 12%) as a white solid.

Tert-butyl (3R,4R)-3-fluoro-4-((methylsulfonyl)oxy)pyrrolidine-1-carboxylate was obtained as follows:

Methanesulfonic anhydride (509 mg, 2.92 mmol) was added to tert-butyl (3R,4R)-3-fluoro-4-hydroxypyrrolidine-1-carboxylate (200 mg, 0.97 mmol) in DCM (4 mL) at 0° C. The resulting mixture was stirred at RT for 1 hour. The reaction mixture was evaporated. Water (25 mL) was added. The reaction mixture was extracted sequentially with DCM (20 mL*3). The combined organic layers were washed with saturated brine (20 mL), dried over Na₂SO₄, filtered and evaporated to afford crude tert-butyl (3R,4R)-3-fluoro-4-((methylsulfonyl)oxy)pyrrolidine-1-carboxylate (0.150 g, 54%). The product was used in the next step directly without further purification.

[D32] was obtained from tert-butyl (S)-3-hydroxypiperidine-1-carboxylate and 4-(9-benzyl-6-(1-methylcyclopropoxy)-9H-purin-8-yl)-3-chlorophenol (Synthetic Example 5, intermediate), using a Mitsunobu reaction conducted at 50° C. for 3 hours using a similar procedure used in the synthesis of Synthetic Example 5, followed by BOC deprotection with HCl in dioxane.

[D33] was obtained from tert-butyl 4-hydroxypiperidine-1-carboxylate and 4-(9-benzyl-6-(1-methylcyclopropoxy)-9H-purin-8-yl)-3-chlorophenol (Synthetic Example 5, intermediate), using a Mitsunobu reaction conducted at 50° C. for 2 hours using a similar procedure used in the synthesis of Synthetic Example 5, followed by BOC deprotection with HCl in dioxane.

[D34] was obtained from tert-butyl 6-hydroxy-2-azaspiro[3.3]heptane-2-carboxylate and 4-(9-benzyl-6-(1-methylcyclopropoxy)-9H-purin-8-yl)-3-chlorophenol (Synthetic Example 5, intermediate), using a Mitsunobu reaction conducted at 50° C. for 2 hours using a similar procedure used in the synthesis of Synthetic Example 5, followed by BOC deprotection with TFA in DCM.

[D35] was obtained from tert-butyl 3-hydroxyazetidine-1-carboxylate and 4-(9-benzyl-6-(1-methylcyclopropoxy)-9H-purin-8-yl)-3-chlorophenol (Synthetic Example 5, intermediate), using a Mitsunobu reaction conducted at 70° C. for 6 hours using a similar procedure used in the synthesis of Synthetic Example 5, followed by BOC deprotection with HCl in dioxane.

The following examples in Table E were synthesised using a similar procedure to 4-(1-benzyl-5-isopropoxy-1H-benzo[d]imidazol-2-yl)-3-chlorophenol (Synthetic Example 7), unless specified differently in the notes at the end of the Table

TABLE E

| | | | | LCMS |
|---|---|---|---|---|
| Ex # | Structure | Name | ¹H NMR | [M + H]+ |
| | 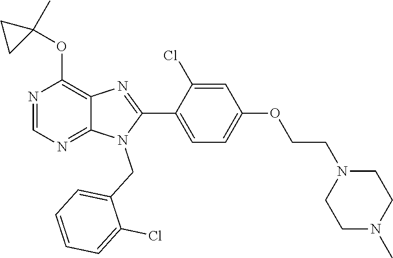 [SMILES: CN1CCN(CCOc2ccc(-c3nc4c(OC5(C)CC5)ncnc4n3Cc3ccccc3Cl)c(Cl)c2)CC1] | 8-(2-chloro-4- (2-(4- methyl- piperazin-1- yl)ethoxy) phenyl)-9-(2- chlorobenzyl)- 6-(1- methylcyclo- propoxy)-9H- purine | (300 MHz, DMSO-d₆): 0.82-0.90 (2H, t), 1.03 (2H, t), 1.74 (3H, s), 2.14 (3H, s), 2.30 (4H, m), 2.50 (4H, m), 2.68 (2H, t), 4.16 (2H, t), 5.40 (2H, s), 6.75 (1H, m), 6.99 (1H, m), 7.14 (1H, m), 7.19-7.29 (2H, m), 7.35-7.43 (2H, m), 8.61 (1H, s) | 567 |
| E2 | 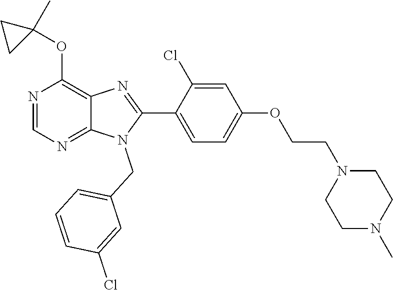 [SMILES: CN1CCN(CCOc2ccc(-c3nc4c(OC5(C)CC5)ncnc4n3Cc3cccc(Cl)c3)c(Cl)c2)CC1] | 8-(2-chloro-4- (2-(4- methyl- piperazin-1- yl)ethoxy) phenyl)-9-(3- chlorobenzyl)- 6-(1- methylcyclo- propoxy)-9H- purine | (300 MHz, DMSO-d₆): 0.86 (2H, t), 1.02 (2H, t), 1.74 (3H, s), 2.15 (3H, s), 2.32 (4H, m), 2.42 (4H, m), 2.71 (2H, t), 4.19 (2H, t), 5.30 (2H, s), 6.85 (1H, m), 6.95 (1H, m), 7.05 (1H, m), 7.20-7.31 (3H, m), 7.43 (1H, d), 8.64 (1H, s) | 567 |
| E3 | 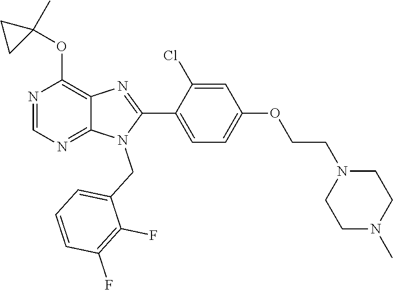 [SMILES: CN1CCN(CCOc2ccc(-c3nc4c(OC5(C)CC5)ncnc4n3Cc3cccc(F)c3F)c(Cl)c2)CC1] | 8-(2-chloro-4- (2-(4- methyl- piperazin-1- yl)ethoxy) phenyl)-9-(2,3- difluoro- benzyl)-6-(1- methylcyclo- propoxy)-9H- purine | (300 MHz, DMSO-d₆): 0.86 (2H, t), 0.99-1.07 (2H, t), 1.73 (3H, s), 2.15 (3H, s), 2.32 (4H, m), 2.42 (4H, m), 2.71 (2H, t), 4.18 (2H, t), 5.39 (2H, s), 6.74 (1H, m), 7.03 (2H, m), 7.22 (1H, d), 7.30 (1H, m), 7.43 (1H, d), 8.64 (1H, s) | 569 |
| E4 | 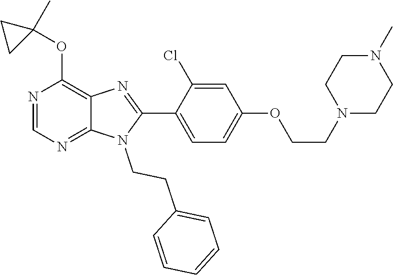 [SMILES: CN1CCN(CCOc2ccc(-c3nc4c(OC5(C)CC5)ncnc4n3CCc3ccccc3)c(Cl)c2)CC1] | 8-(2-chloro-4- (2-(4- methyl- piperazin-1- yl)ethoxy) phenyl)-6-(1- methylcyclo- propoxy)-9- phenethyl-9H- purine | (400 MHz, DMSO-d₆): 0.80-0.87 (2H, m), 0.98- 1.04 (2H, m), 1.73 (3H, s), 2.15 (3H, s), 2.32 (4H, s), 2.54 (4H, s), 2.71 (2H, t), 2.98 (2H, t), 4.19 (2H, t), 4.25 (2H, t), 6.82 (2H, dd), 6.94-7.04 (2H, m), 7.16 (3H, dd), 7.26 (1H, d), 8.61 (1H, s) | 547 |
| E5 | 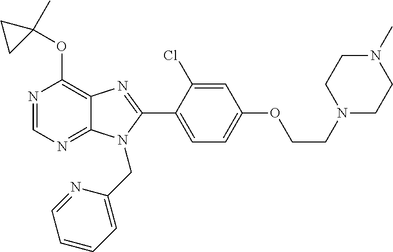 [SMILES: CN1CCN(CCOc2ccc(-c3nc4c(OC5(C)CC5)ncnc4n3Cc3ccccn3)c(Cl)c2)CC1] | 8-(2-chloro-4- (2-(4- methyl- piperazin-1- yl)ethoxy) phenyl)-6-(1- methylcyclo- propoxy)-9- (pyridin-2- ylmethyl)-9H- purine | (400 MHz, DMSO-d₆): 0.75-0.88 (2H, m), 0.95- 1.07 (2H, m), 1.71 (3H, s), 2.11 (3H, s), 2.28 (4H, s), 2.45 (3H, s), 2.51 (1H, t), 2.65 (2H, s), 4.12 (2H, t), 5.38 (2H, s), 6.95 (1H, dd), 7.04 (1H, d), 7.15-7.22 (2H, m), 7.37 (1H, d), 7.63 (1H, td), 8.31 (1H, dt), 8.54 (1H, s) | 534 |
| E6 | 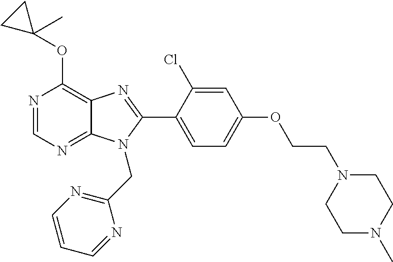 [SMILES: CN1CCN(CCOc2ccc(-c3nc4c(OC5(C)CC5)ncnc4n3Cc3ncccn3)c(Cl)c2)CC1] | 8-(2-chloro-4- (2-(4- methyl- piperazin-1- yl)ethoxy) phenyl)-6-(1- methylcyclo- propoxy)-9- (pyrimidin-2- ylmethyl)-9H- purine | (400 MHz, DMSO-d₆): 0.78-0.93 (2H, m), 0.96- 1.12 (2H, m), 1.75 (3H, s), 2.17 (3H, s), 2.28-2.41 (4H, m), 2.41-2.50 (4H, m), 2.68 (2H, t), 4.14 (2H, t), 5.49 (2H, s), 6.94 (1H, dd), 7.21 (1H, d), 7.33 (1H, d), 7.36 (1H, t), 8.20 (1H, s), 8.55 (1H, s), 8.64 (2H, d). 1H not observed. | 535 |
| E7 | 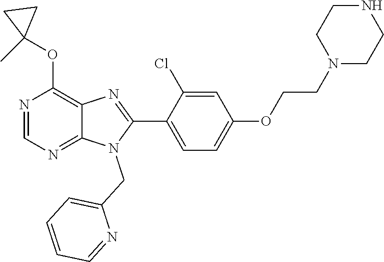 [SMILES: CC1(Oc2ncnc3c2nc(-c2ccc(OCCN4CCNCC4)cc2Cl)n3Cc2ccccn2)CC1] | 8-(2-chloro-4- (2-(piperazin- 1- yl)ethoxy) phenyl)-6-(1- methylcyclo- propoxy)-9- (pyridin-2- ylmethyl)-9H- purine | (300 MHz, DMSO-d₆): 0.79-0.89 (2H, m), 1.02 (2H, d), 1.73 (3H, s), 2.42 (4H, s), 2.66 (2H, t), 2.73 (4H, t), 4.14 (2H, t), 5.39 (2H, s), 6.96 (1H, dd), 7.06 (1H, d), 7.17-7.23 (2H, m), 7.37 (1H, d), 7.65 (1H, td), 8.31-8.35 (1H, m), 8.56 (1H, s). One proton not observed. | 520 |

[E1] was made following a similar procedure to 4-((8-(2-chloro-4-(2-(4-methylpiperazin-1-yl)ethoxy)phenyl)-6-(1-methylcyclopropoxy)-9H-purin-9-yl)methyl)-2-methylthiazole (Synthetic Example 7), using N-(2-chlorobenzyl)-6-(1-methylcyclopropoxy)-5-nitropyrimidin-4-amine and 2-chloro-4-(2-(4-methylpiperazin-1-yl)ethoxy)benzaldehyde as starting materials.

N-(2-chlorobenzyl)-6-(1-methylcyclopropoxy)-5-nitropyrimidin-4-amine was made following a similar procedure to 6-(1-methylcyclopropoxy)-N-((2-methylthiazol-4-yl)methyl)-5-nitropyrimidin-4-amine (Synthetic Example 7, intermediate) using 6-chloro-N-(2-chlorobenzyl)-5-nitropyrimidin-4-amine and 1-methylcyclopropan-1-ol as starting material. ¹H NMR (300 MHz, DMSO-d₆): 0.76 (2H, t), 0.91 (2H, t), 1.61 (3H, s), 4.73 (2H, d), 7.22-7.29 (3H, in), 7.39-7.47 (JH, in), 8.29 (JH, s), 8.84 (JH, t). m/z: ES+[M+H]+ 335.

6-Chloro-N-(2-chlorobenzyl)-5-nitropyrimidin-4-amine was made following a similar procedure to 6-chloro-N-((2-methylthiazol-4-yl)methyl)-5-nitropyrimidin-4-amine (Synthetic Example 7, intermediate). ¹H NMR (300 MHz, DMSO-d₆): 4.72 (2H, d), 7.23-7.33 (3H, in), 7.40-7.49 (JH, in), 8.42 (JH, s), 9.00 (JH, t). m/z: ES+[M+H]+ 299.

[E2] was made following a similar procedure to 4-((8-(2-chloro-4-(2-(4-methylpiperazin-1-yl)ethoxy)phenyl)-6-(1-methylcyclopropoxy)-9H-purin-9-yl)methyl)-2-methylthiazole (Synthetic Example 7), using N-(3-chlorobenzyl)-6-(1-methylcyclopropoxy)-5-nitropyrimidin-4-amine and 2-chloro-4-(2-(4-methylpiperazin-1-yl)ethoxy)benzaldehyde as starting materials.

N-(3-Chlorobenzyl)-6-(1-methylcyclopropoxy)-5-nitropyrimidin-4-amine was made following a similar procedure to 6-(1-methylcyclopropoxy)-N-((2-methylthiazol-4-yl)methyl)-5-nitropyrimidin-4-amine (Synthetic Example 7, intermediate), using 6-chloro-N-(3-chlorobenzyl)-5-nitropyrimidin-4-amine and 1-methylcyclopropan-1-ol as starting materials. ¹H NMR (300 MHz, DMSO-d₆): 0.73-0.8 (2H, m), 0.93 (2H, t), 1.63 (3H, s), 4.69 (2H, d), 7.26-7.4 (4H, m), 8.34 (1H, s), 8.88 (1H, t). m/z: ES+[M+H]+ 335.

6-Chloro-N-(3-chlorobenzyl)-5-nitropyrimidin-4-amine was made from (3-chlorophenyl)methanamine and 4,6-dichloro-5-nitropyrimidine following a similar procedure to 6-chloro-N-((2-methylthiazol-4-yl)methyl)-5-nitropyrimidin-4-amine (Synthetic Example 7, intermediate). ¹H NMR (300 MHz, DMSO-d₆): 4.69 (2H, d), 7.18-7.51 (4H, m), 8.46 (1H, d), 9.02 (1H, t). m/z: ES+[M+H]+ 299.

[E3] was made following a similar procedure to 4-((8-(2-chloro-4-(2-(4-methylpiperazin-1-yl)ethoxy)phenyl)-6-(1-methylcyclopropoxy)-9H-purin-9-yl)methyl)-2-methylthiazole (Synthetic Example 7), using N-(2,3-difluorobenzyl)-6-(1-methylcyclopropoxy)-5-nitropyrimidin-4-amine and 2-chloro-4-(2-(4-methylpiperazin-1-yl)ethoxy)benzaldehyde as starting material.

N-(2,3-Difluorobenzyl)-6-(1-methylcyclopropoxy)-5-nitropyrimidin-4-amine was made following a similar procedure to 6-(1-methylcyclopropoxy)-N-((2-methylthiazol-4-yl)methyl)-5-nitropyrimidin-4-amine (Synthetic Example 7, intermediate), using 6-chloro-N-(2,3-difluorobenzyl)-5-nitropyrimidin-4-amine and 1-methylcyclopropan-1-ol as starting materials. ¹H NMR (300 MHz, DMSO-d₆): 0.75 (2H, t), 0.90 (2H, t), 1.60 (3H, s), 4.75 (2H, d), 7.06-7.19 (2H, m), 7.22-7.34 (1H, m), 8.31 (1H, s), 8.84 (1H, t). m/z: ES+[M+H]+ 337.

6-Chloro-N-(2,3-difluorobenzyl)-5-nitropyrimidin-4-amine was using a similar procedure to 6-chloro-N-(2-chlorobenzyl)-5-nitropyrimidin-4-amine (Synthetic Example 7, intermediate), using 4,6-dichloro-5-nitropyrimidine and (2,3-difluorophenyl)methanamine as starting materials. ¹H NMR (300 MHz, DMSO-d₆): 4.73 (2H, d), 6.95-7.22 (2H, m), 7.22-7.42 (1H, m), 8.45 (1H, s), 8.82-9.15 (1H, m). m/z: ES+[M+H]+ 301.

[E4] was made following a similar procedure to 4-((8-(2-chloro-4-(2-(4-methylpiperazin-1-yl)ethoxy)phenyl)-6-(1-methylcyclopropoxy)-9H-purin-9-yl)methyl)-2-methylthiazole (Synthetic Example 7), using 6-(1-methylcyclopropoxy)-5-nitro-N-phenethylpyrimidin-4-amine and 2-chloro-4-(2-(4-methylpiperazin-1-yl)ethoxy)benzaldehyde as starting materials.

6-(1-Methylcyclopropoxy)-5-nitro-N-phenethylpyrimidin-4-amine was made following a similar procedure to 6-(1-methylcyclopropoxy)-N-((2-methylthiazol-4-yl)methyl)-5-nitropyrimidin-4-amine (Synthetic Example 7, intermediate), using 6-chloro-5-nitro-N-phenethylpyrimidin-4-amine and 1-methylcyclopropan-1-ol as starting materials. ¹H NMR (300 MHz, DMSO-d₆): 0.72-0.80 (2H, m), 0.89-0.96 (2H, m), 1.62 (3H, s), 2.86 (2H, t), 3.71 (2H, dt), 7.17-7.26 (3H, m), 7.26-7.33 (2H, m), 8.36 (1H, s), 8.41 (1H, t). m/z: ES+[M+H]+ 315.

6-Chloro-5-nitro-N-phenethylpyrimidin-4-amine was made using a similar procedure to 6-chloro-N-(3-chlorobenzyl)-5-nitropyrimidin-4-amine (Synthetic Example 7, intermediate), using 2-phenylethan-1-amine and 4,6-dichloro-5-nitropyrimidine as starting materials. ¹H NMR (300 MHz, DMSO-d₆): 2.86 (2H, t), 3.67-3.85 (2H, m), 7.13-7.33 (5H, m), 8.07 (1H, s), 9.56 (1H, t). m/z: ES+[M+H]+ 279.

[E5] was made following a similar procedure to 4-((8-(2-chloro-4-(2-(4-methylpiperazin-1-yl)ethoxy)phenyl)-6-(1-methylcyclopropoxy)-9H-purin-9-yl)methyl)-2-methylthiazole (Synthetic Example 7), using 6-(1-methylcyclopropoxy)-5-nitro-N-(pyridin-2-ylmethyl)pyrimidin-4-amine and 2-chloro-4-(2-(4-methylpiperazin-1-yl)ethoxy)benzaldehyde as starting materials.

6-(1-Methylcyclopropoxy)-5-nitro-N-(pyridin-2-ylmethyl)pyrimidin-4-amine was made following a similar procedure to 6-(1-methylcyclopropoxy)-N-((2-methylthiazol-4-yl)methyl)-5-nitropyrimidin-4-amine (Synthetic Example 7, intermediate), using 6-chloro-5-nitro-N-(pyridin-2-ylmethyl)pyrimidin-4-amine and 1-methylcyclopropan-1-ol as starting material. ¹H NMR (300 MHz, DMSO-d₆): 0.79 (2H, dd), 0.87-1.03 (2H, m), 1.64 (2H, s), 4.82 (2H, d), 7.27-7.43 (2H, m), 7.75-7.85 (1H, m), 8.33 (1H, s), 8.55 (1H, dd), 9.06 (1H, t). One proton not observed. m/z: ES+[M+H]+ 302.

6-Chloro-5-nitro-N-(pyridin-2-ylmethyl)pyrimidin-4-amine was made using a similar procedure to 6-chloro-N-(3-chlorobenzyl)-5-nitropyrimidin-4-amine (Synthetic Example 7, intermediate), using 4,6-dichloro-5-nitropyrimidine and pyridin-2-ylmethanamine as starting materials. m/z: ES+[M+H]+ 266.

[E6] was made following a similar procedure to 4-((8-(2-chloro-4-(2-(4-methylpiperazin-1-yl)ethoxy)phenyl)-6-(1-methylcyclopropoxy)-9H-purin-9-yl)methyl)-2-methylthiazole (Synthetic Example 7), using 6-(1-methylcyclopropoxy)-5-nitro-N-(pyrimidin-2-ylmethyl)pyrimidin-4-amine and 2-chloro-4-(2-(4-methylpiperazin-1-yl)ethoxy)benzaldehyde as starting materials and purified by preparative HPLC (Viridis BEH Prep 2-EP OBD column, 30*150 mm, 5 μm), using decreasingly polar mixtures of water (containing formic acid) and MeCN as eluents to isolate the desired compound as the formate salt.

6-(1-Methylcyclopropoxy)-5-nitro-N-(pyrimidin-2-ylmethyl)pyrimidin-4-amine was made following a similar procedure to 6-(1-methylcyclopropoxy)-N-((2-methylthiazol-4-yl)methyl)-5-nitropyrimidin-4-amine (Synthetic Example 7, intermediate) using 6-chloro-5-nitro-N-(pyrimidin-2-ylmethyl)pyrimidin-4-amine and 1-methylcyclopropan-1-ol as starting materials. ¹H NMR (300 MHz, DMSO-d₆): 0.61-0.69 (2H, m), 0.80-0.87 (2H, m), 1.52 (3H, s), 4.77 (2H, d), 7.30 (1H, t), 8.18 (1H, s), 8.66 (2H, d), 8.90 (1H, s). m/z: ES+[M+H]+ 303.

6-Chloro-5-nitro-N-(pyrimidin-2-ylmethyl)pyrimidin-4-amine was made using a similar procedure to 6-chloro-N-(2-chlorobenzyl)-5-nitropyrimidin-4-amine (Synthetic Example 7, intermediate), from 4,6-dichloro-5-nitropyrimidine and pyrimidin-2-ylmethanamine as starting material. ¹H NMR (300 MHz, DMSO-d₆): 4.87 (2H, d), 7.33 (1H, t), 8.70 (2H, d), 10.06 (1H, t), 12.36 (1H, s). m/z: ES+[M+H]+ 267.

[E7] was made following a similar procedure to 9-benzyl-8-(2-chloro-4-(2-(piperazin-1-yl)ethoxy)phenyl)-6-((1,1,1-trifluoro-2-methylpropan-2-yl)oxy)-9H-purine (Synthetic Example 11), using tert-butyl 4-(2-(3-chloro-4-(6-(1-methylcyclopropoxy)-9-(pyridin-2-ylmethyl)-9H-purin-8-yl)phenoxy)ethyl)piperazine-1-carboxylate as starting material.

Tert-butyl 4-(2-(3-chloro-4-(6-(1-methylcyclopropoxy)-9-(pyridin-2-ylmethyl)-9H-purin-8-yl)phenoxy)ethyl)piperazine-1-carboxylate was made following a similar procedure to 4-((8-(2-chloro-4-(2-(4-methylpiperazin-1-yl)ethoxy)phenyl)-6-(1-methylcyclopropoxy)-9H-purin-9-yl)methyl)-2-methylthiazole (Synthetic Example 7), using 6-(1-methylcyclopropoxy)-5-nitro-N-(pyridin-2-ylmethyl)pyrimidin-4-amine (previously described in Synthetic Example E5) and tert-butyl 4-(2-(3-chloro-4-formylphenoxy)ethyl)piperazine-1-carboxylate as starting materials.

The following examples in Table F were made in a similar manner to Synthetic Example 19, except stated otherwise in the notes at the bottom of the Table

TABLE F

| | | | | LCMS |
|---|---|---|---|---|
| Ex# | Structure | Name | ¹H NMR | [M + H]+ |
| F1 | 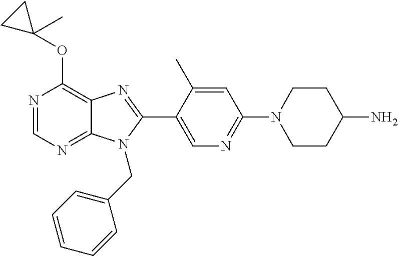 [SMILES: Cc1cc(N2CCC(N)CC2)ncc1-c1nc2c(OC3(C)CC3)ncnc2n1Cc1ccccc1] | 1-(5-(9-benzyl- 6-(1- methylcyclo- propoxy)-9H- purin-8-yl)-4- methylpyridin- 2-yl)piperidin- 4-amine | (400 MHz, DMSO-d₆): 0.80-0.87 (2H, m), 1.00- 1.03 (2H, m), 1.19-1.22 (2H, m), 1.73-1.78 (5H, m), 1.96 (3H, s), 2.95 (4H, t), 3.51 (1H, s), 4.25 (2H, d), 5.33 (2H, s), 6.78 (1H, s), 6.89 (2H, dd), 7.17-7.29 (3H, m), 8.01 (1H, s), 8.58 (1H, s) | 470 |
| F2 | 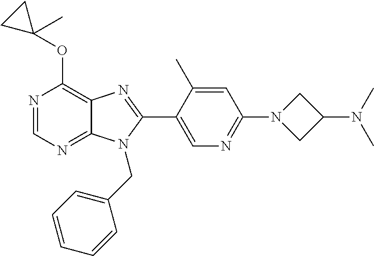 [SMILES: Cc1cc(N2CC(N(C)C)C2)ncc1-c1nc2c(OC3(C)CC3)ncnc2n1Cc1ccccc1] | 1-(5-(9-benzyl- 6-(1- methylcyclo- propoxy)-9H- purin-8-yl)-4- methylpyridin- 2-yl)-N,N- dimethyl- azetidin-3- amine | (400 MHz, DMSO-d₆): 0.81-0.87 (2H, m), 0.99- 1.05 (2H, m), 1.73 (3H, s), 1.93 (3H, s), 2.12 (6H, s), 3.19 (1H, ddd), 3.76 (2H, dd), 4.02 (2H, dd), 5.31 (2H, s), 6.31 (1H, s), 6.89 (2H, dd), 7.22 (3H, dd), 7.98 (1H, s), 8.58 (1H, s) | 470 |
| F3 | 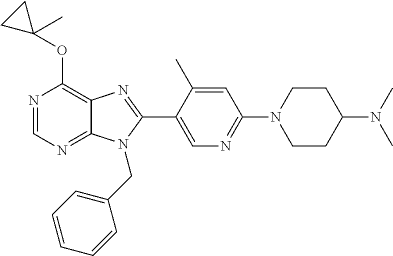 [SMILES: Cc1cc(N2CCC(N(C)C)CC2)ncc1-c1nc2c(OC3(C)CC3)ncnc2n1Cc1ccccc1] | 1-(5-(9-benzyl- 6-(1- methylcyclo- propoxy)-9H- purin-8-yl)-4- methylpyridin- 2-yl)-N,N- dimethyl- piperidin-4- amine | (400 MHz, DMSO-d₆): 0.81-0.87 (2H, m), 0.98- 1.06 (2H, m), 1.33 (2H, qd), 1.73 (3H, s), 1.80 (2H, d), 1.95 (3H, s), 2.18 (6H, s), 2.26-2.39 (1H, m), 2.86 (2H, t), 4.36 (2H, d), 5.33 (2H, s), 6.79 (1H, s), 6.86-6.92 (2H, m), 7.22 (3H, dd), 8.01 (1H, s), 8.58 (1H, s) | 498 |

[F1] was made following a similar procedure to 1-(5-(9-benzyl-6-(1-methylcyclopropoxy)-9H-purin-8-yl)-4-methylpyridin-2-yl)azetidin-3-amine (Synthetic Example 19), using tert-butyl piperidin-4-ylcarbamate (185 mg, 0.92 mmol) and 9-benzyl-8-(6-fluoro-4-methylpyridin-3-yl)-6-(1-methylcyclopropoxy)-9H-purine (180 mg, 0.46 mmol) to afford 1-(5-(9-benzyl-6-(1-methylcyclopropoxy)-9H-purin-8-yl)-4-methylpyridin-2-yl)piperidin-4-amine (22 mg, 13%) as a light yellow solid.

[F2] was made following a similar procedure to 1-(5-(9-benzyl-6-(1-methylcyclopropoxy)-9H-purin-8-yl)-4-methylpyridin-2-yl)azetidin-3-amine (Synthetic Example 19) using N,N-dimethyl-3-azetidinamine (089 mg, 0.51 mmol) and 9-benzyl-8-(6-fluoro-4-methylpyridin-3-yl)-6-(1-methylcyclopropoxy)-9H-purine (100 g, 0.26 mmol) to afford 1-(5-(9-benzyl-6-(1-methylcyclopropoxy)-9H-purin-8-yl)-4-methylpyridin-2-yl)-N,N-dimethylazetidin-3-amine (37 mg, 31%) as a light yellow solid, except that the BOC deprotection step was omitted.

[F3] was made following a similar procedure to 1-(5-(9-benzyl-6-(1-methylcyclopropoxy)-9H-purin-8-yl)-4-methylpyridin-2-yl)azetidin-3-amine (Synthetic Example 19) using N,N-dimethylpiperidin-4-amine (66 mg, 0.51 mmol) and 9-benzyl-8-(6-fluoro-4-methylpyridin-3-yl)-6-(1-methylcyclopropoxy)-9H-purine (100 mg, 0.26 mmol) to afford 1-(5-(9-benzyl-6-(1-methylcyclopropoxy)-9H-purin-8-yl)-4-methylpyridin-2-yl)-N,N-dimethylpiperidin-4-amine (33 mg, 26%) as a light yellow solid, except that the BOC deprotection step was omitted

Unless stated otherwise, the following examples in Table G were made by a similar method to Synthetic Example 21. Where BOC deprotection was required, this step was performed by a method similar to that described in Synthetic Example 19.

TABLE G

| | | | | LCMS |
|---|---|---|---|---|
| Ex# | Structure | Name | ¹H NMR | [M + H]+ |
| G1 | 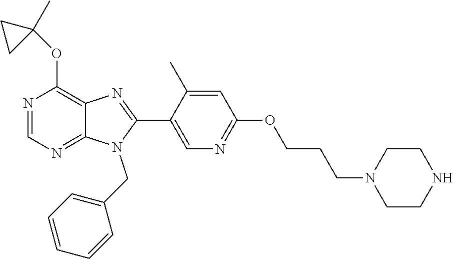 [SMILES: Cc1cc(OCCCN2CCNCC2)ncc1-c1nc2c(OC3(C)CC3)ncnc2n1Cc1ccccc1] | 9-benzyl-8-(4- methyl-6-(3- (piperazin-1- yl)propoxy) pyridin-3-yl)-6- (1-methylcyclo- propoxy)- 9H-purine | (400 MHz, DMSO-d₆): 0.81-0.89 (2H, m), 0.99-1.07 (2H, m), 1.73 (3H, s), 1.87 (2H, p), 1.92 (3H, s), 2.29 (4H, br s), 2.37 (2H, t), 2.67 (4H, t), 4.31 (2H, t), 5.32 (2H, s), 6.78 (1H, s), 6.88 (2H, dd), 7.16-7.26 (3H, m), 8.11 (1H, s), 8.63 (1H, s). 1H not observed. | 514 |
| G2 | 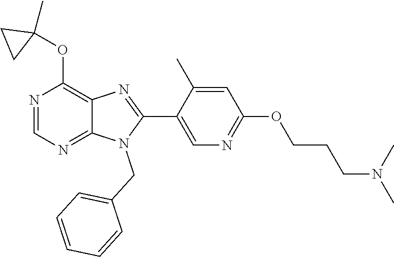 [SMILES: Cc1cc(OCCCN(C)C)ncc1-c1nc2c(OC3(C)CC3)ncnc2n1Cc1ccccc1] | 3-((5-(9-benzyl- 6-(1-methyl- cyclopropoxy)- 9H-purin-8- yl)-4- methylpyridin-2- yl)oxy)-N,N- dimethylpropan- 1-amine | (400 MHz, DMSO-d₆): 0.80-0.88 (2H, m), 1.01-1.04 (2H, m), 1.73 (3H, s), 1.85 (2H, p), 1.92 (3H, s), 2.14 (6H, s), 2.34 (2H, t), 4.31 (2H, t), 5.33 (2H, s), 6.78 (1H, s), 6.88 (2H, dd), 7.21 (3H, p), 8.11 (1H, s), 8.63 (1H, s) | 473 |
| G3 | 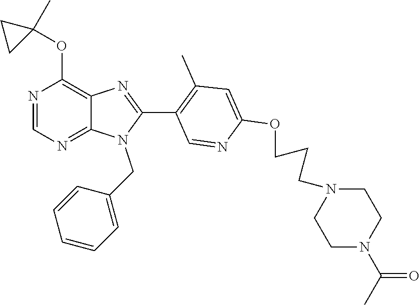 [SMILES: CC(=O)N1CCN(CCCOc2cc(C)c(-c3nc4c(OC5(C)CC5)ncnc4n3Cc3ccccc3)cn2)CC1] | 1-(4-(3-((5-(9- benzyl-6-(1- methylcyclo- propoxy)-9H- purin-8-yl)-4- methylpyridin-2- yl)oxy)propyl) piperazin-1- yl)ethan-1-one | (400 MHz, DMSO-d₆): 0.81-0.89 (2H, m), 0.99-1.06 (2H, m), 1.73 (3H, s), 1.90-1.93 (5H, m), 1.98 (3H, s), 2.31 (2H, br s), 2.38 (2H, br s), 2.43 (2H, t), 3.30-3.50 (4H, m), 4.33 (2H, t), 5.33 (2H, s), 6.78 (1H, s), 6.86-6.92 (2H, m), 7.20-7.22 (3H, m), 8.12 (1H, s), 8.63 (1H, s) | 556 |
| G4 | 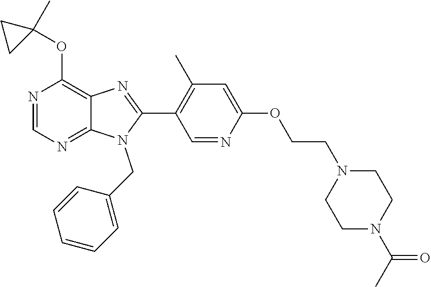 [SMILES: CC(=O)N1CCN(CCOc2cc(C)c(-c3nc4c(OC5(C)CC5)ncnc4n3Cc3ccccc3)cn2)CC1] | 1-(4-(2-((5-(9- benzyl-6-(1- methylcyclo- propoxy)-9H- purin-8-yl)-4- methylpyridin-2- yl)oxy)ethyl) piperazin-1-yl) ethan-1-one | (400 MHz, DMSO-d₆): 0.79-0.89 (2H, m), 0.97-1.08 (2H, m), 1.73 (3H, s), 1.93 (3H, s), 1.98 (3H, s), 2.42 (2H, t), 2.48 (2H, d), 2.72 (2H, t), 3.41 (4H, q), 4.43 (2H, t), 5.33 (2H, s), 6.80 (1H, s), 6.88 (2H, dd), 7.15-7.26 (3H, m), 8.12 (1H, s), 8.63 (1H, s) | 542 |
| G5 | 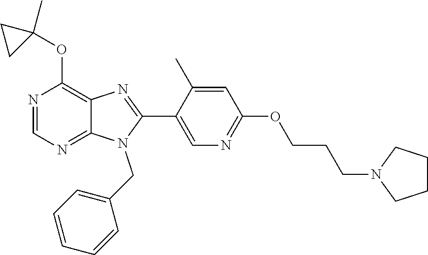 [SMILES: Cc1cc(OCCCN2CCCC2)ncc1-c1nc2c(OC3(C)CC3)ncnc2n1Cc1ccccc1] | 9-benzyl-8-(4- methyl-6-(3- (pyrrolidin-1- yl)propoxy) pyridin-3-yl)-6- (1-methylcyclo- propoxy)-9H- purine | (400 MHz, DMSO-d₆): 0.79-0.89 (2H, m), 0.98-1.08 (2H, m), 1.67 (4H, p), 1.73 (3H, s), 1.85-1.91 (2H, m), 1.92 (3H, s), 2.41-2.44 (4H, m), 4.33 (2H, t), 5.32 (2H, s), 6.78 (1H, s), 6.84- 6.93 (2H, m), 7.19- 7.22 (3H, m), 8.11 (1H, s), 8.63 (1H, s). 2H not observed. | 499 |
| G6 | 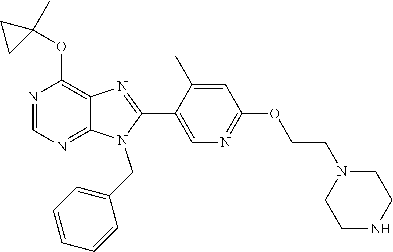 [SMILES: Cc1cc(OCCN2CCNCC2)ncc1-c1nc2c(OC3(C)CC3)ncnc2n1Cc1ccccc1] | 9-benzyl-8-(4- methyl-6-(2- (piperazin-1- yl)ethoxy)pyridin- 3-yl)-6-(1- methylcyclo- propoxy)-9H- purine | (400 MHz, DMSO-d₆): 0.80-0.88 (2H, m), 0.98-1.06 (2H, m), 1.73 (3H, s), 1.92 (3H, s), 2.39 (4H, t), 2.61- 2.71 (6H, m), 4.40 (2H, t), 5.32 (2H, s), 6.79 (1H, s), 6.84-6.92 (2H, m), 7.17-7.25 (3H, m), 8.11 (1H, s), 8.63 (1H, s). 1H not observed. | 500 |
| G7 | 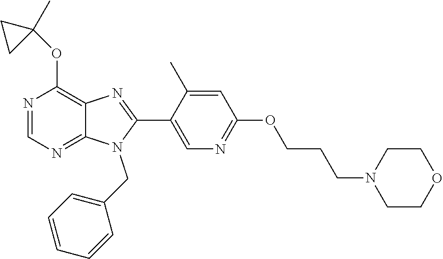 [SMILES: Cc1cc(OCCCN2CCOCC2)ncc1-c1nc2c(OC3(C)CC3)ncnc2n1Cc1ccccc1] | 4-(3-((5-(9- benzyl-6-(1- methylcyclo- propoxy)-9H- purin-8-yl)-4- methylpyridin-2- yl)oxy)propyl) morpholine | (400 MHz, DMSO-d₆): 0.80-0.88 (2H, m), 1.00-1.04 (2H, m), 1.73 (3H, s), 1.85-1.96 (5H, m), 2.28-2.46 (6H, m), 3.55-3.58 (4H, m), 4.33 (2H, t), 5.32 (2H, s), 6.78 (1H, s), 6.88 (2H, dd), 7.20-7.22 (3H, m), 8.11 (1H, s), 8.63 (1H, s) | 515 |
| G8 | 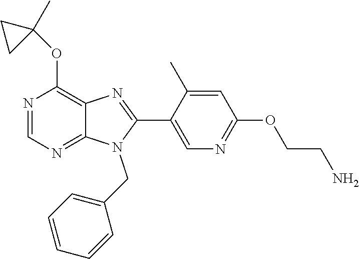 [SMILES: Cc1cc(OCCN)ncc1-c1nc2c(OC3(C)CC3)ncnc2n1Cc1ccccc1] | 2-((5-(9-benzyl- 6-(1-methyl- cyclopropoxy)- 9H-purin-8- yl)-4- methylpyridin-2- yl)oxy)ethan-1- amine | (400 MHz, DMSO-d₆): 0.80-0.89 (2H, m), 0.98-1.07 (2H, m), 1.73 (3H, s), 1.80 (2H, br s), 1.93 (3H, s), 2.88 (2H, t), 4.24 (2H, t), 5.33 (2H, s), 6.79 (1H, s), 6.85-6.98 (2H, m), 7.14-7.30 (3H, m), 8.11 (1H, s), 8.63 (1H, s) | 431 |
| G9 | 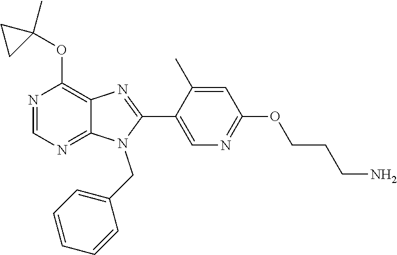 [SMILES: Cc1cc(OCCCN)ncc1-c1nc2c(OC3(C)CC3)ncnc2n1Cc1ccccc1] | 3-((5-(9-benzyl- 6-(1-methyl- cyclopropoxy)- 9H-purin-8- yl)-4- methylpyridin-2- yl)oxy)propan-1- amine | (400 MHz, DMSO-d₆): 0.80-0.88 (2H, m), 0.99-1.06 (2H, m), 1.73 (3H, s), 1.79 (2H, p), 1.93 (3H, s), 2.69 (2H, t), 4.35 (2H, t), 5.33 (2H, s), 6.78 (1H, s), 6.84-6.93 (2H, m), 7.17-7.26 (3H, m), 8.11 (1H, s), 8.62 (1H, s), 2H not observed. | 445 |
| G10 | 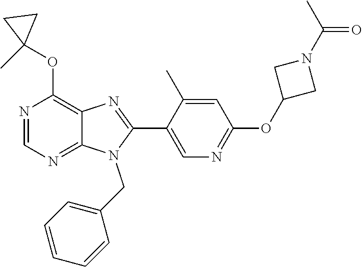 [SMILES: CC(=O)N1CC(Oc2cc(C)c(-c3nc4c(OC5(C)CC5)ncnc4n3Cc3ccccc3)cn2)C1] | 1-(3-((5-(9- benzyl-6-(1- methylcyclo- propoxy)-9H- purin-8-yl)-4- methylpyridin-2- yl)oxy)azetidin- 1-yl)ethan-1-one | (400 MHz, DMSO-d₆): 0.81-0.93 (2H, m), 1.01-1.04 (2H, m), 1.73 (3H, s), 1.80 (3H, s), 1.93 (3H, s), 3.78- 3.86 (1H, m), 4.07- 4.15 (1H, m), 4.23- 4.27 (1H, m), 4.50- 4.58 (1H, m), 5.32 (2H, s), 5.37 (1H, tt), 6.85-6.88 (3H, m), 7.17-7.25 (3H, m), 8.13 (1H, s), 8.63 (1H, s) | 485 |
| G11 | 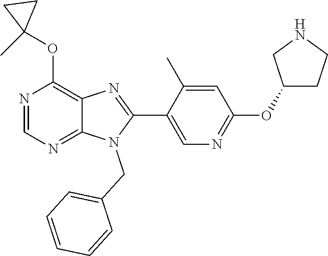 [SMILES: Cc1cc(O[C@H]2CCNC2)ncc1-c1nc2c(OC3(C)CC3)ncnc2n1Cc1ccccc1] | (S)-9-benzyl-8- (4-methyl-6- (pyrrolidin-3- yloxy)pyridin-3- yl)-6-(1-methyl- cyclopropoxy)- 9H-purine | (400 MHz, DMSO-d₆): 0.81-0.88 (2H, m), 1.01-1.04 (2H, m), 1.73 (3H, s), 1.74-1.80 (1H, m), 1.92 (3H, s), 2.01-2.05 (1H, m), 2.76-2.87 (2H, m), 2.88-2.93 (1H, m), 3.08 (1H, dd), 5.32 (2H, s), 5.36-5.45 (1H, m), 6.74 (1H, s), 6.83-6.92 (2H, m), 7.15-7.26 (3H, m), 8.11 (1H, s), 8.63 (1H, s). 1H not observed. | 457 |
| G12 | 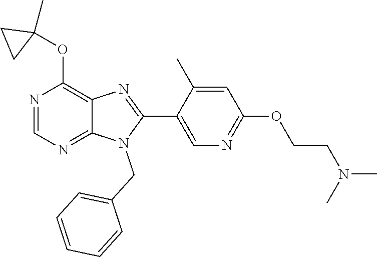 [SMILES: Cc1cc(OCCN(C)C)ncc1-c1nc2c(OC3(C)CC3)ncnc2n1Cc1ccccc1] | 2-((5-(9-benzyl- 6-(1-methyl- cyclopropoxy)- 9H-purin-8- yl)-4- methylpyridin-2- yl)oxy)-N,N- dimethylethan-1- amine | (400 MHz, DMSO-d₆): 0.80-0.88 (2H, m), 1.02 (2H, t), 1.73 (3H, s), 1.92 (3H, s), 2.21 (6H, s), 2.62 (2H, t), 4.38 (2H, t), 5.33 (2H, s), 6.78 (1H, s), 6.88 (2H, dd), 7.21 (3H, q), 8.12 (1H, s), 8.63 (1H, s). | 459 |
| G13 | 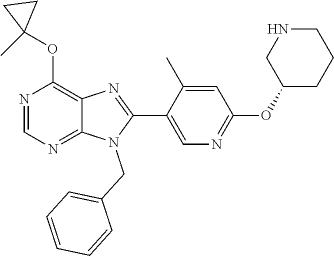 [SMILES: Cc1cc(O[C@H]2CCCNC2)ncc1-c1nc2c(OC3(C)CC3)ncnc2n1Cc1ccccc1] | (S)-9-benzyl-8- (4-methyl-6- (piperidin-3- yloxy)pyridin-3- yl)-6-(1-methyl- cyclopropoxy)- 9H-purine | (400 MHz, DMSO-d₆): 0.81-0.88 (2H, m), 0.98-1.07 (2H, m), 1.37-1.61 (2H, m), 1.63-1.72 (1H, m), 1.73 (3H, s), 1.93 (3H, s), 2.02-2.10 (1H, m), 2.26 (1H, br s), 2.52-2.55 (1H, m), 2.67- 2.77 (1H, m), 3.13 (1H, dd), 4.93-4.96 (1H, m), 5.33 (2H, s), 6.73 (1H, s), 6.84- 6.93 (2H, m), 7.16- 7.28 (3H, m), 8.09 (1H, s), 8.62 (1H, s). | 471 |
| | | | 1H not observed. | |
| G14 | 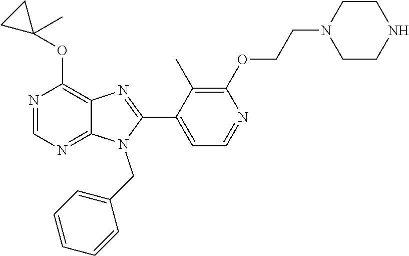 [SMILES: Cc1c(-c2nc3c(OC4(C)CC4)ncnc3n2Cc2ccccc2)ccnc1OCCN1CCNCC1] | 9-benzyl-8-(3- methyl-2-(2- (piperazin-1- yl)ethoxy)pyridin- 4-yl)-6-(1-methyl- cyclopropoxy)- 9H-purine | (400 MHz, DMSO-d₆): 0.81-0.88 (2H, m), 1.02 (2H, m), 1.73 (3H, s), 1.74 (3H, s) 2.39 (4H, br s), 2.65-2.68 (6H, m), 4.43 (2H, t), 5.30 (2H, s), 6.83- 6.90 (2H, m), 6.98 (1H, d), 7.20 (3H, dd), 8.10 (1H, d), 8.65 (1H, s). 1H not observed. | 500 |
| G15 | 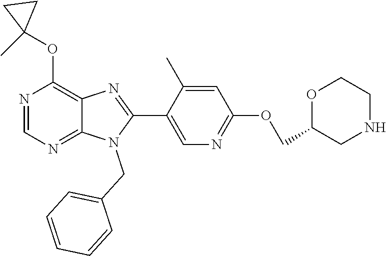 [SMILES: Cc1cc(OC[C@H]2CNCCO2)ncc1-c1nc2c(OC3(C)CC3)ncnc2n1Cc1ccccc1] | (R)-2-(((5-(9- benzyl-6-(1- methylcyclo- propoxy)-9H- purin-8-yl)-4- methylpyridin-2- yl)oxy)methyl) morpholine | (400 MHz, DMSO-d₆): 0.82-0.88 (2H, m), 0.99-1.06 (2H, m), 1.73 (3H, s), 1.92 (3H, s), 2.46-2.47 (1H, m), 2.65 (2H, dd), 2.84 (1H, dd), 3.45 (1H, td), 3.64-3.80 (2H, m), 4.17-4.31 (2H, m), 5.32 (2H, s), 6.81 (1H, s), 6.88 (2H, dd), 7.15-7.27 (3H, m), 8.11 (1H, s), 8.63 (1H, s). 1H not observed. | 487 |
| G16 | 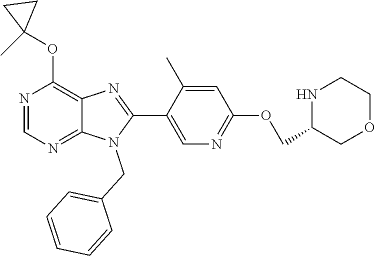 [SMILES: Cc1cc(OC[C@H]2COCCN2)ncc1-c1nc2c(OC3(C)CC3)ncnc2n1Cc1ccccc1] | (R)-3-(((5-(9- benzyl-6-(1- methylcyclo- propoxy)-9H- purin-8-yl)-4- methylpyridin-2- yl)oxy)methyl) morpholine | (400 MHz, DMSO-d₆): 0.80-0.92 (2H, m), 1.00-1.04 (2H, m), 1.73 (3H, s), 1.93 (3H, s), 2.56 (1H, s), 2.69- 2.83 (2H, m), 3.05 (1H, dtd), 3.22 (1H, dd), 3.38 (1H, td), 3.66 (1H, dt), 3.79 (1H, dd), 4.09-4.24 (2H, m), 5.33 (2H, s), 6.80 (1H, s), 6.84-6.92 (2H, m), 7.21 (3H, ddt), 8.11 (1H, s), 8.63 (1H, s) | 487 |
| G17 | 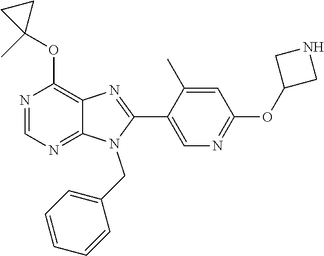 [SMILES: Cc1cc(OC2CNC2)ncc1-c1nc2c(OC3(C)CC3)ncnc2n1Cc1ccccc1] | 8-{6-[(azetidin-3- yl)oxy]-4- methylpyridin-3- yl}-9-benzyl-6- [(1-methyl- cyclopropyl) oxy]-9H-purine | (400 MHz, DMSO-d₆): 0.80-0.88 (2H, m), 0.98-1.06 (2H, m), 1.73 (3H, s), 1.91 (3H, s), 3.52 (2H, dd), 3.71- 3.80 (2H, m), 5.31 (2H, s), 5.37 (1H, p), 6.80 (1H, s), 6.82-6.89 (2H, m), 7.15-7.25 (3H, m), 8.09 (1H, s), 8.63 (1H, s). 1H not observed. | 443 |

[G1] made by a similar procedure to Synthetic Example 21 using tert-butyl 4-(3-hydroxypropyl)piperazine-1-carboxylate (94 mg, 0.39 mmol) and 9-benzyl-8-(6-fluoro-4-methylpyridin-3-yl)-6-(1-methylcyclopropoxy)-9H-purine (150 mg, 0.39 mmol), except that the reaction was quenched with saturated aq. NH₄Cl (20 mL), extracted with EtOAc (3×20 mL). The organic layer was dried over Na₂SO₄, filtered and evaporated to afford 0.22 g yellow solid which was used in the BOC deprotection step directly without further purification.

[G3] made via acetylation of 9-benzyl-8-(4-methyl-6-(3-(piperazin-1-yl)propoxy)pyridin-3-yl)-6-(1-methylcyclopropoxy)-9H-purine (Synthetic Example G1)

Ac₂O (24 μL, 0.25 mmol) was added to 9-benzyl-8-(4-methyl-6-(3-(piperazin-1-yl)propoxy)pyridin-3-yl)-6-(1-methylcyclopropoxy)-9H-purine (Synthetic Example G1, 130 mg, 0.25 mmol) and DIEA (133 μL, 0.76 mmol) in DCM (3 mL). The resulting mixture was stirred at rt for 1 hour. The solvent was removed under reduced pressure. The crude product was purified by preparative HPLC (XBridge Prep OBD column, 30*150 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness to afford 1-(4-(3-((5-(9-benzyl-6-(1-methylcyclopropoxy)-9H-purin-8-yl)-4-methylpyridin-2-yl)oxy)propyl)piperazin-1-yl)ethan-1-one (45 mg, 32%) as a light yellow solid.

[G6] made by a similar procedure to Synthetic Example 21 using tert-butyl 4-(2-hydroxyethyl)piperazine-1-carboxylate (177 mg, 0.77 mmol) and 9-benzyl-8-(6-fluoro-4-methylpyridin-3-yl)-6-(1-methylcyclopropoxy)-9H-purine (150 mg, 0.39 mmol), except that the reaction was quenched with saturated aq. NH₄Cl (20 mL), extracted with EtOAc (3×20 mL), the organic layer was dried over Na₂SO₄, filtered and evaporated to afford 0.22 g yellow solid which was used in the BOC deprotection step directly without further purification.

[G8] made by a similar procedure to Synthetic Example 21 using tert-butyl (2-hydroxyethyl)carbamate (248 mg, 1.54 mmol) and 9-benzyl-8-(6-fluoro-4-methylpyridin-3-yl)-6-(1-methylcyclopropoxy)-9H-purine (300 mg, 0.77 mmol), except that the reaction mixture was quenched with saturated NH₄Cl (20 mL), extracted with EtOAc (3×20 mL), the organic layer was dried over Na₂SO₄, filtered and evaporated to afford 0.4 g yellow solid which was used in the BOC deprotection step directly without further purification.

[G9] made by a similar procedure to Synthetic Example 21 using tert-butyl (3-hydroxypropyl)carbamate (135 mg, 0.77 mmol) and 9-benzyl-8-(6-fluoro-4-methylpyridin-3-yl)-6-(1-methylcyclopropoxy)-9H-purine (150 mg, 0.39 mmol), except that the reaction mixture was quenched with saturated NH₄Cl (20 mL), extracted with EtOAc (3×20 mL), the organic layer was dried over Na₂SO₄, filtered and evaporated to afford crude 0.22 g as a yellow solid, which was used in the BOC deprotection step directly without further purification.

[G11] made by a similar procedure to Synthetic Example 21 tert-butyl (S)-3-hydroxypyrrolidine-1-carboxylate (48.1 mg, 0.26 mmol) and 9-benzyl-8-(6-fluoro-4-methylpyridin-3-yl)-6-(1-methylcyclopropoxy)-9H-purine (100 mg, 0.26 mmol), except that the reaction mixture was quenched with saturated NH₄Cl (20 mL), extracted with EtOAc (3×20 mL), the organic layer was dried over Na₂SO₄, filtered and evaporated to afford crude 0.15 g yellow solid, which was used in the BOC deprotection step directly without further purification.

[G12] was made from 2-((5-(9-benzyl-6-(1-methylcyclopropoxy)-9H-purin-8-yl)-4-methylpyridin-2-yl)oxy)ethan-1-amine (Synthetic Example G8) as follows:

Formaldehyde (4 mL, 0.33 mmol. 37% aqueous solution) was added to 2-((5-(9-benzyl-6-(1-methylcyclopropoxy)-9H-purin-8-yl)-4-methylpyridin-2-yl)oxy)ethan-1-amine (140 mg, 0.33 mmol) in MeOH (2 mL). The resulting mixture was stirred at rt for 20 minutes. Then sodium triacetoxyborohydride (414 mg, 1.95 mmol) was added to the mixture. The resulting mixture was stirred at 60° C. for 16 hours. The solvent was removed under reduced pressure. The crude product was purified by preparative HPLC (XBridge Prep OBD C18 column, 30*150 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness to afford 2-((5-(9-benzyl-6-(1-methylcyclopropoxy)-9H-purin-8-yl)-4-methylpyridin-2-yl)oxy)-N,N-dimethylethan-1-amine (31 mg, 21%) as a light yellow solid.

[G13] made by a similar procedure to Synthetic Example 21 using tert-butyl (S)-3-hydroxypiperidine-1-carboxylate (116 mg, 0.58 mmol) and 9-benzyl-8-(6-fluoro-4-methylpyridin-3-yl)-6-(1-methylcyclopropoxy)-9H-purine (150 mg, 0.39 mmol), except that the reaction mixture was quenched with saturated NH₄Cl (20 mL), extracted with EtOAc (3×20 mL), the organic layer was dried over Na₂SO₄, filtered and evaporated to afford crude 0.20 g yellow solid, which was used in the BOC deprotection step directly without further purification.

[G14] made by a similar procedure to Synthetic Example 21 using tert-butyl 4-(2-hydroxyethyl)piperazine-1-carboxylate (237 mg, 1.03 mmol) and 9-benzyl-8-(2-fluoro-3-methylpyridin-4-yl)-6-(1-methylcyclopropoxy)-9H-purine (200 mg, 0.51 mmol), except that the reaction mixture was quenched with saturated NH₄Cl (20 mL), extracted with EtOAc (3×20 mL), the organic layer was dried over Na₂SO₄, filtered and evaporated to afford crude 0.30 g yellow solid, which was used in the BOC deprotection step directly without further purification.

9-Benzyl-8-(2-fluoro-3-methylpyridin-4-yl)-6-(1-methylcyclopropoxy)-9H-purine used as a starting material was obtained from a similar procedure as that described for 9-benzyl-8-(6-fluoro-4-methylpyridin-3-yl)-6-(1-methylcyclopropoxy)-9H-purine (Synthetic Example 19 intermediate) using N4-benzyl-6-(1-methylcyclopropoxy)pyrimidine-4,5-diamine (200 mg, 0.74 mmol) and 2-fluoro-3-methylisonicotinaldehyde (124 mg, 0.89 mmol) to afford to afford 9-benzyl-8-(2-fluoro-3-methylpyridin-4-yl)-6-(1-methylcyclopropoxy)-9H-purine (0.160 g, 55%) as a brown solid. ¹H NMR (400 MHz, DMSO-d₆): 0.86 (2H, t), 1.02 (2H, t), 1.73 (3H, s), 1.84 (3H, s), 5.35 (2H, s), 6.87 (2H, dd), 7.20 (3H, dd), 7.43 (1H, d), 8.20 (1H, d), 8.68 (1H, d). m/z: ES+[M+H]+ 390.

[G15] made by a similar procedure to Synthetic Example 21 using tert-butyl (R)-2-(hydroxymethyl)morpholine-4-carboxylate (117 mg, 0.54 mmol) and 9-benzyl-8-(6-fluoro-4-methylpyridin-3-yl)-6-(1-methylcyclopropoxy)-9H-purine (150 mg, 0.39 mmol), except that the reaction mixture was quenched with saturated NH₄Cl (20 mL), extracted with EtOAc (3×20 mL), the organic layer was dried over Na₂SO₄, filtered and evaporated to afford crude 0.15 g as a yellow solid, which was used in the BOC deprotection step directly without further purification.

[G16] made by a similar procedure to Synthetic Example 21 using tert-butyl (S)-3-(hydroxymethyl)morpholine-4-carboxylate (669 mg, 3.08 mmol) and 9-benzyl-8-(6-fluoro-4-methylpyridin-3-yl)-6-(1-methylcyclopropoxy)-9H-purine (300 mg, 0.77 mmol) as starting material. After the BOC deprotection step, the compound was purified by flash C18-flash chromatography, elution gradient 5 to 80% MeCN in water (containing 0.1% NH₄HCO₃), followed by preparative HPLC (XBridge Prep OBD C18 column, 30*150 mm, 5 μm), using decreasingly polar mixtures of water (containing 10 mmol/L NH₄HCO₃ and 0.1% aqueous ammonia) and MeCN as eluents. Fractions containing the BOC deprotected product were evaporated to dryness to afford (R)-3-(((5-(9-benzyl-6-(1-methylcyclopropoxy)-9H-purin-8-yl)-4-methylpyridin-2-yl)oxy)methyl)morpholine (21.0 mg, 5.6%) as a light yellow solid.

[G17] made by a similar procedure to Synthetic Example 21 using tert-butyl 3-hydroxyazetidine-1-carboxylate (67 mg, 0.39 mmol) and 9-benzyl-8-(6-fluoro-4-methylpyridin-3-yl)-6-(1-methylcyclopropoxy)-9H-purine (100 mg, 0.26 mmol), except that the reaction mixture was quenched with saturated NH₄C1 (20 mL), extracted with EtOAc (3×20 mL). The organic layer was dried over Na₂SO₄, filtered and evaporated to afford crude 0.12 g yellow solid, which was used in the BOC deprotection step directly without further purification.

The following examples in Table H were synthesised as stated in the notes at the bottom of the Table.

TABLE H

| | | | | LCMS |
|---|---|---|---|---|
| Ex# | Structure | Name | ¹H NMR | [M + H]+ |
| H1 | 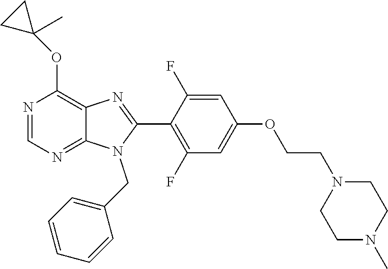 [SMILES: CN1CCN(CCOc2cc(F)c(-c3nc4c(OC5(C)CC5)ncnc4n3Cc3ccccc3)c(F)c2)CC1] | 9-benzyl-8-(2,6- difluoro-4-(2- (4-methyl- piperazin-1- yl)ethoxy) phenyl)-6-(1- methylcyclo- propoxy)-9H- purine | (400 MHz, DMSO-d₆): 0.81-0.88 (2H, t), 1.00- 1.07 (2H, t), 1.73 (3H, s), 2.15 (3H, s), 2.23-2.39 (4H, m), 2.49 (4H, m), 2.70 (2H, t), 4.18 (2H, t), 5.33 (2H, s), 6.89-7.00 (4H, m), 7.19-7.24 (3H, m), 8.64 (1H, s) | 535 |
| H2 | 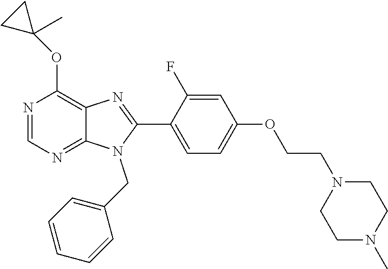 [SMILES: CN1CCN(CCOc2ccc(-c3nc4c(OC5(C)CC5)ncnc4n3Cc3ccccc3)c(F)c2)CC1] | 9-benzyl-8-(2- fluoro-4-(2-(4- methylpiperazin- 1-yl)ethoxy) phenyl)-6-(1- methylcyclo- propoxy)-9H- purine | (400 MHz, DMSO-d₆): 0.85 (2H, s), 1.03 (2H, s), 1.73 (3H, s), 2.15 (3H, s), 2.22-2.38 (4H, m), 2.42 (4H, m), 2.71 (2H, m), 4.17 (2H, s), 5.38 (2H, s), 6.91 (3H, m), 7.06 (1H, d), 7.21 (3H, s), 7.47 (1H, t), 8.60 (1H, s). | 517 |
| H3 | 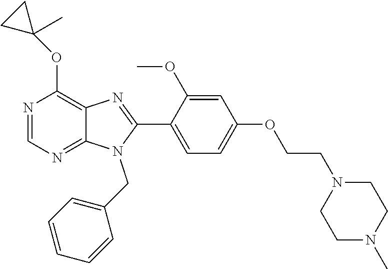 [SMILES: COc1cc(OCCN2CCN(C)CC2)ccc1-c1nc2c(OC3(C)CC3)ncnc2n1Cc1ccccc1] | 9-benzyl-8-(2- methoxy-4-(2- (4-methyl- piperazin-1- yl)ethoxy) phenyl)-6-(1- methylcyclo- propoxy)-9H- purine | (400 MHz, DMSO-d₆): 0.84 (2H, s), 1.01 (2H, s), 1.73 (3H, s), 2.16 (3H, s), 2.22-2.40 (4H, m), 2.67-2.74 (2H, m), 3.67 (3H, s), 4.16 (2H, s), 5.26 (2H, s), 6.62-6.67 (1H, d), 6.72 (1H, s), 6.87- 6.94 (2H, m), 7.20 (3H, m), 7.27 (1H, d), 8.55 (1H, s) 4 H not observed | 529 |
| H4 | 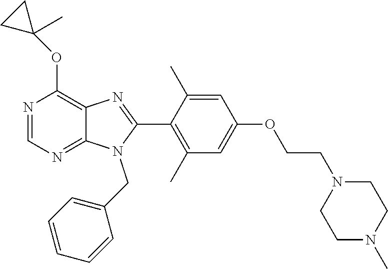 [SMILES: Cc1cc(OCCN2CCN(C)CC2)cc(C)c1-c1nc2c(OC3(C)CC3)ncnc2n1Cc1ccccc1] | 9-benzyl-8-(2,6- dimethyl-4-(2- (4- methylpiperazin- 1-yl)ethoxy) phenyl)-6-(1- methylcyclo- propoxy)-9H- purine | (400 MHz, DMSO-d₆): 0.78-0.88 (2H, m), 0.98- 1.08 (2H, m), 1.70 (6H, s), 1.73 (3H, s), 2.15 (3H, s), 2.23-2.41 (4H, m), 2.54 (4H, s), 2.70 (2H, t), 4.12 (2H, t), 5.09 (2H, s), 6.75 (2H, s), 6.85- 6.93 (2H, m), 7.14- 7.28 (3H, m), 8.65 (1H, s). | 527 |
| H5 | 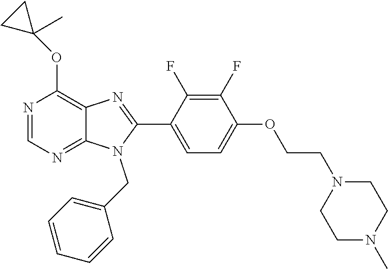 [SMILES: CN1CCN(CCOc2ccc(-c3nc4c(OC5(C)CC5)ncnc4n3Cc3ccccc3)c(F)c2F)CC1] | 9-benzyl-8-(2,3- difluoro-4-(2- (4- methylpiperazin- 1-yl)ethoxy) phenyl)-6-(1- methylcyclo- propoxy)-9H- purine | (400 MHz, DMSO-d₆): 0.81-0.88 (2H, m), 0.99- 1.06 (2H, m), 1.73 (3H, s), 2.14 (3H, s), 2.30 (4H, s), 2.47-2.52 (4H, m), 2.73 (2H, t), 4.27 (2H, t), 5.41 (2H, s), 6.88- 6.95 (2H, m), 7.21 (4H, td), 7.33 (1H, td), 8.62 (1H, s). | 535 |
| H6 | 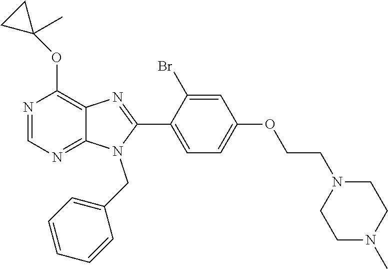 [SMILES: CN1CCN(CCOc2ccc(-c3nc4c(OC5(C)CC5)ncnc4n3Cc3ccccc3)c(Br)c2)CC1] | 9-benzyl-8-(2- bromo-4-(2-(4- methylpiperazin- 1-yl)ethoxy) phenyl)-6-(1- methylcyclo- propoxy)-9H- purine | (400 MHz, DMSO-d₆): 0.88 (2H, m), 1.02 (2H, s), 1.73 (3H, s), 2.14 (3H, s), 2.33 (4H, dt), 2.48 (4H, s), 2.70 (2H, t), 4.18 (2H, t), 5.27 (2H, s), 6.91 (2H, dd), 7.06 (1H, dd), 7.20 (3H, ddt), 7.32-7.42 (2H, m), 8.61 (1H, s). | 577 |
| H7 | 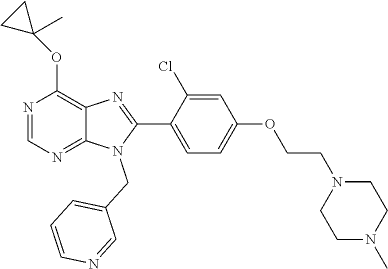 [SMILES: CN1CCN(CCOc2ccc(-c3nc4c(OC5(C)CC5)ncnc4n3Cc3cccnc3)c(Cl)c2)CC1] | 8-(2-chloro-4- (2-(4- methylpiperazin- 1-yl)ethoxy) phenyl)-6-(1- methylcyclo- propoxy)-9- (pyridin-3- ylmethyl)-9H- purine | (400 MHz, DMSO-d₆): 0.85 (2H, d), 0.98-1.07 (2H, m), 1.73 (3H, s), 2.14 (3H, s), 2.25-2.38 (4H, m), 2.45 (4H, d), 2.70 (2H, t), 4.18 (2H, t), 5.32 (2H, s), 7.06 (1H, dd), 7.19-7.28 (2H, m), 7.34 (1H, dt), 7.45 (1H, d), 8.16 (1H, d), 8.41 (1H, dd), 8.62 (1H, s) | 534 |
| H8 | 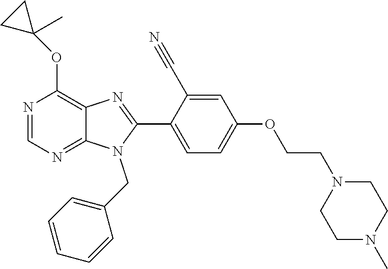 [SMILES: CN1CCN(CCOc2ccc(-c3nc4c(OC5(C)CC5)ncnc4n3Cc3ccccc3)c(C#N)c2)CC1] | 2-(9-benzyl-6- (1-methylcyclo- propoxy)-9H- purin-8-yl)-5- (2-(4- methylpiperazin- 1-yl)ethoxy) benzonitrile | (400 MHz, DMSO-d₆): 0.86 (2H, d), 1.03 (2H, d), 1.74 (3H, s), 2.14 (3H, s), 2.23-2.37 (4H, m), 2.47 (4H, d), 2.70 (2H, t), 4.22 (2H, t), 5.43 (2H, s), 6.88 (2H, dd), 7.17- 7.27 (3H, m), 7.39 (1H, dd), 7.62 (1H, d), 7.67 (1H, d), 8.65 (1H, s). | 524 |
| H9 | 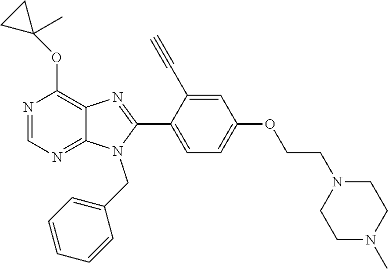 [SMILES: C#Cc1cc(OCCN2CCN(C)CC2)ccc1-c1nc2c(OC3(C)CC3)ncnc2n1Cc1ccccc1] | 9-benzyl-8-(2- ethynyl-4-(2-(4- methylpiperazin- 1-yl)ethoxy) phenyl)-6-(1- methylcyclo- propoxy)-9H- purine | (400 MHz, DMSO-d₆): 0.80-0.87 (2H, m), 0.98- 1.05 (2H, m), 1.73 (3H, s), 2.14 (3H, s), 2.31 (4H, s), 2.49 (4H, d), 2.69 (2H, t), 4.17 (2H, t), 4.22 (1H, s), 5.35 (2H, s), 6.85-6.90 (2H, m), 7.09 (1H, dd), 7.18 (3H, ddt), 7.24 (1H, d), 7.35 (1H, d), 8.59 (1H, s). | 523 |
| H10 | 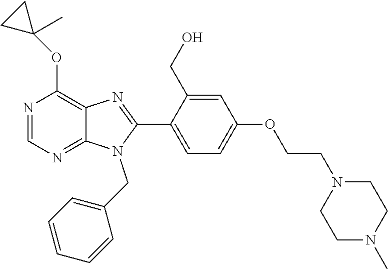 [SMILES: CN1CCN(CCOc2ccc(-c3nc4c(OC5(C)CC5)ncnc4n3Cc3ccccc3)c(CO)c2)CC1] | (2-(9-benzyl-6- (1-methylcyclo- propoxy)-9H- purin-8-yl)-5- (2-(4- methylpiperazin- 1-yl)ethoxy) phenyl) methanol | (400 MHz, DMSO-d₆): 0.80-0.89 (2H, m), 0.98- 1.09 (2H, m), 1.73 (3H, s), 2.14 (3H, s), 2.32 (4H, s), 2.47 (4H, d), 2.71 (2H, t), 4.14 (2H, t), 4.26 (2H, s), 5.19 (1H, s), 5.30 (2H, s), 6.86-6.97 (3H, m), 7.15-7.27 (5H, m), 8.58 (1H, s). | 529 |
| H11 | 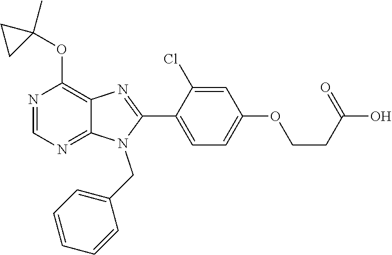 [SMILES: CC1(Oc2ncnc3c2nc(-c2ccc(OCCC(=O)O)cc2Cl)n3Cc2ccccc2)CC1] | 3-(4-(9-benzyl- 6-(1- methylcyclo- propoxy)-9H- purin-8-yl)-3- chlorophenoxy) propanoic acid | (400 MHz, DMSO-d₆): 0.80-0.92 (2H, m), 1.01 (2H, d), 1.73 (3H, s), 2.68 (2H, t), 4.27 (2H, t), 5.29 (2H, s), 6.90 (2H, dd), 7.02 (1H, dd), 7.16-7.25 (4H, m), 7.40 (1H, d), 8.62 (1H, s). One proton not observed. | 479 |
| H12 | 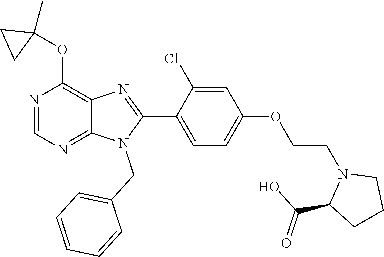 [SMILES: CC1(Oc2ncnc3c2nc(-c2ccc(OCCN4CCC[C@H]4C(=O)O)cc2Cl)n3Cc2ccccc2)CC1] | (2-(4-(9-benzyl- 6-(1-methyl- cyclopropoxy)- 9H-purin-8-yl)-3- chlorophenoxy) ethyl)-L-proline | (400 MHz, DMSO-d₆): 0.73 (2H, t), 0.88 (2H, t), 1.56-1.64 (4H, m), 1.7- 1.85 (2H, m), 1.98-2.1 (1H, m), 2.7-2.83 (1H, m), 3.08-3.19 (1H, m), 3.24-3.34 (2H, m), 3.45- 3.52 (1H, m), 4.19 (2H, t), 5.17 (2H, s), 6.77 (2H, dd), 6.92 (1H, dd), 7.06-7.13 (4H, m), 7.30 (1H, d), 8.48 (1H, s). One proton not observed. | 548 |
| H13 | 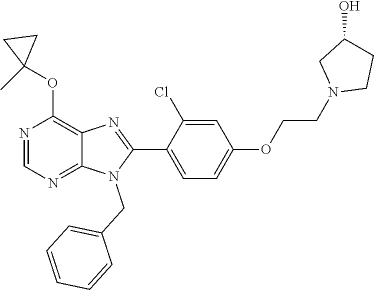 [SMILES: CC1(Oc2ncnc3c2nc(-c2ccc(OCCN4CC[C@@H](O)C4)cc2Cl)n3Cc2ccccc2)CC1] | (R)-1-(2-(4-(9- benzyl-6-(1- methylcyclo- propoxy)-9H- purin-8-yl)-3- chlorophenoxy) ethyl)pyrrolidin- 3-ol | (400 MHz, DMSO-d₆): 0.84 (2H, t), 1.01 (2H, t), 1.48-1.58 (1H, m), 1.73 (3H, s), 1.91-2.03 (1H, m), 2.37-2.43 (1H, m), 2.51- 2.55 (1H, m), 2.61-2.7 (1H, m), 2.73-2.82 (3H, m), 4.12-4.23 (3H, m), 4.69 (1H, d), 5.29 (2H, s), 6.85-6.92 (2H, m), 7.03 (1H, dd), 7.18- 7.22 (3H, m), 7.24 (1H, d), 7.40 (1H, d), 8.61 (1H, s). | 520 |
| H14 | 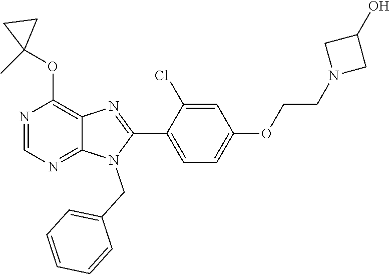 [SMILES: CC1(Oc2ncnc3c2nc(-c2ccc(OCCN4CC(O)C4)cc2Cl)n3Cc2ccccc2)CC1] | 1-(2-(4-(9- benzyl-6-(1- methylcyclo- propoxy)-9H- purin-8-yl)-3- chlorophenoxy) ethyl)azetidin- 3-ol | (400 MHz, DMSO-d₆): 0.84 (2H, t), 1.02 (2H, t), 1.73 (3H, s), 2.68-2.86 (4H, m), 3.51-3.62 (2H, m), 4.03 (2H, t), 4.11- 4.23 (1H, m), 5.21-5.34 (3H, m), 6.86-6.94 (2H, m), 6.97-7.05 (1H, m), 7.15-7.26 (4H, m), 7.40 (1H, d), 8.61 (1H, s). | 506 |
| H15 | 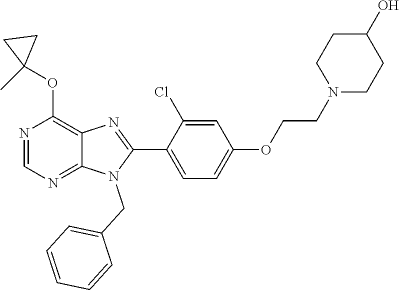 [SMILES: CC1(Oc2ncnc3c2nc(-c2ccc(OCCN4CCC(O)CC4)cc2Cl)n3Cc2ccccc2)CC1] | 1-(2-(4-(9- benzyl-6-(1- methylcyclo- propoxy)-9H- purin-8-yl)-3- chlorophenoxy) ethyl)piperidin- 4-ol | (400 MHz, DMSO-d₆): 0.84 (2H, t), 1.01 (2H, t), 1.34-1.43 (2H, m), 1.67- 1.75 (5H, m), 2.08-2.18 (2H, m), 2.68 (2H, t), 2.74- 2.83 (2H, m), 3.38-3.5 (1H, m), 4.16 (2H, t), 4.54 (1H, d), 5.29 (2H, s), 6.89-6.92 (2H, m), 7.03 (1H, dd), 7.18-7.22 (3H, m), 7.25 (1H, d), 7.40 (1H, d), 8.61 (1H, s). | 534 |
| H16 | 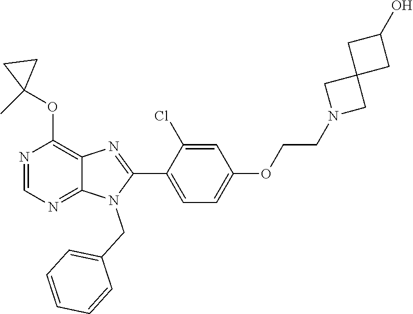 [SMILES: CC1(Oc2ncnc3c2nc(-c2ccc(OCCN4CC5(CC(O)C5)C4)cc2Cl)n3Cc2ccccc2)CC1] | 2-(2-(4-(9- benzyl-6-(1- methylcyclo- propoxy)-9H- purin-8-yl)-3- chlorophenoxy) ethyl)-2- azaspiro[3.3] heptan-6-ol | (400 MHz, DMSO-d₆): 0.85 (2H, t), 1.01 (2H, t), 1.73 (3H, s), 1.81-1.91 (2H, m), 2.27-2.35 (2H, m), 2.69 (2H, t), 3.09- 3.17 (4H, m), 3.89-3.95 (1H, m), 4.00 (2H, t), 4.92 (1H, t), 5.28 (2H, s), 6.85-6.93 (2H, m), 6.99 (1H, dd), 7.17-7.22 (4H, m), 7.39 (1H, d), 8.61 (1H, s). | 546 |
| H17 | 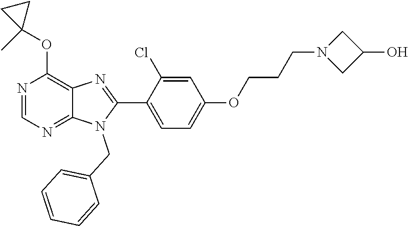 [SMILES: CC1(Oc2ncnc3c2nc(-c2ccc(OCCCN4CC(O)C4)cc2Cl)n3Cc2ccccc2)CC1] | 1-(3-(4-(9- benzyl-6-(1- methylcyclo- propoxy)-9H- purin-8-yl)-3- chlorophenoxy) propyl)azetidin- 3-ol | (400 MHz, DMSO-d₆): 0.84 (2H, t), 1.01 (2H, t), 1.70-1.77 (5H, m), 2.46- 2.5 (2H, m), 2.59-2.73 (2H, m), 3.43-3.56 (2H, m), 4.07 (2H, t), 4.10- 4.21 (1H, m), 5.12-5.39 (3H, m), 6.86-6.92 (2H, m), 7.00-7.08 (1H, m), 7.16-7.23 (4H, m), 7.39 (1H, d), 8.61 (1H, s). | 520 |
| H18 | 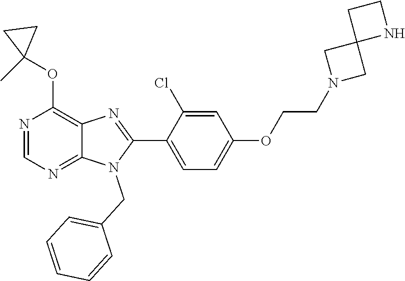 [SMILES: CC1(Oc2ncnc3c2nc(-c2ccc(OCCN4CC5(CCN5)C4)cc2Cl)n3Cc2ccccc2)CC1] | 8-(4-(2-(1,6- diazaspiro[3.3] heptan-6- yl)ethoxy)-2- chlorophenyl)- 9-benzyl-6-(1- methylcyclo- propoxy)-9H- purine | (300 MHz, CD₃OD): 0.87 (2H, t), 1.12 (2H, t), 1.81 (3H, s), 2.55 (2H, t), 2.90 (2H, t), 3.33-3.36 (2H, m), 3.44 (2H, t), 3.62-3.73 (2H, m), 4.10 (2H, t), 5.39 (2H, s), 6.87-6.94 (2H, m), 6.91-6.99 (1H, m), 7.16-7.21 (4H, m), 7.24 (1H, d), 8.62 (1H, s). One proton not observed. | 531 |
| H19 | 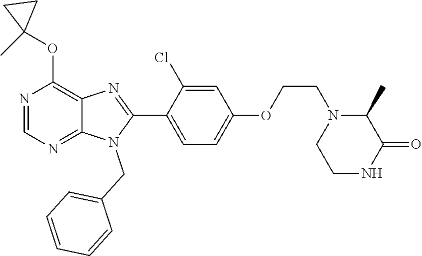 [SMILES: C[C@H]1C(=O)NCCN1CCOc1ccc(-c2nc3c(OC4(C)CC4)ncnc3n2Cc2ccccc2)c(Cl)c1] | (S)-4-(2-(4-(9- benzyl-6-(1- methylcyclo- propoxy)-9H- purin-8-yl)-3- chlorophenoxy) ethyl)-3- methylpiperazin- 2-one | (300 MHz, DMSO-d₆): 0.85 (2H, t), 1.01 (2H, t), 1.24 (3H, d), 1.73 (3H, s), 2.58-2.66 (1H, m), 2.77- 2.83 (1H, m), 2.94-3.03 (2H, m), 3.09-3.17 (3H, m), 4.18-4.23 (2H, m), 5.29 (2H, s), 6.87- 6.93 (2H, m), 7.04 (1H, dd), 7.19-7.22 (3H, m), 7.26 (1H, d), 7.41 (1H, d), 7.69 (1H, s), 8.62 (1H, s). | 547 |
| H20 | 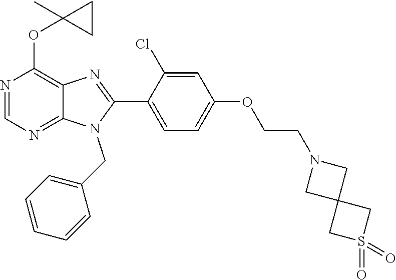 [SMILES: CC1(Oc2ncnc3c2nc(-c2ccc(OCCN4CC5(C4)CS(=O)(=O)C5)cc2Cl)n3Cc2ccccc2)CC1] | 6-(2-(4-(9- benzyl-6-(1- methylcyclo- propoxy)-9H- purin-8-yl)-3- chlorophenoxy) ethyl)-2-thia-6- azaspiro[3.3] heptane 2,2- dioxide | (400 MHz, DMSO-d₆): 0.85 (2H, t), 1.01 (2H, t), 1.73 (3H, s), 2.78 (2H, t), 3.39-3.45 (4H, m), 4.05 (2H, t), 4.32 (4H, s), 5.29 (2H, s), 6.88-6.92 (2H, m), 6.98-7.04 (1H, m), 7.19-7.22 (4H, m), 7.41 (1H, d), 8.61 (1H, s). | 580 |
| H21 | 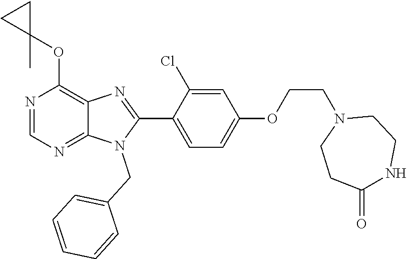 [SMILES: CC1(Oc2ncnc3c2nc(-c2ccc(OCCN4CCNC(=O)CC4)cc2Cl)n3Cc2ccccc2)CC1] | 1-(2-(4-(9- benzyl-6-(1- methylcyclo- propoxy)-9H- purin-8-yl)-3- chlorophenoxy) ethyl)-1,4- diazepan-5-one | (400 MHz, DMSO-d₆): 0.85 (2H, t), 1.01 (2H, t), 1.73 (3H, s), 2.38-2.44 (2H, m), 2.6-2.68 (4H, m), 2.85 (2H, t), 3.07-3.17 (2H, m), 4.19 (2H, t), 5.29 (2H, s), 6.89-6.93 (2H, m), 7.04 (1H, dd), 7.17-7.23 (3H, m), 7.25 (1H, d), 7.41 (1H, d), 7.54 (1H, t), 8.61 (1H, s). | 547 |
| H22 | 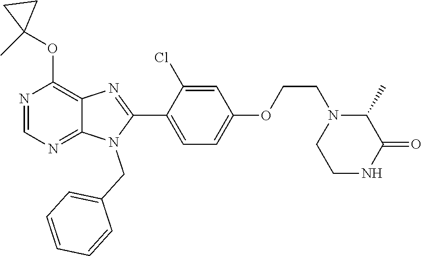 [SMILES: C[C@@H]1C(=O)NCCN1CCOc1ccc(-c2nc3c(OC4(C)CC4)ncnc3n2Cc2ccccc2)c(Cl)c1] | (R)-4-(2-(4-(9- benzyl-6-(1- methylcyclo- propoxy)-9H- purin-8-yl)-3- chlorophenoxy) ethyl)-3- methylpiperazin- 2-one | (400 MHz, DMSO-d₆): 0.84 (2H, t), 1.01 (2H, t), 1.24 (3H, d), 1.73 (3H, s), 2.57-2.67 (1H, m), 2.76- 2.84 (1H, m), 2.93-3.03 (2H, m), 3.06-3.16 (3H, m), 4.17-4.23 (2H, m), 5.29 (2H, s), 6.87- 6.92 (2H, m), 7.04 (1H, dd), 7.18-7.21 (3H, m), 7.26 (1H, d), 7.41 (1H, d), 7.67 (1H, s), 8.61 (1H, s) | 547 |
| H23 | 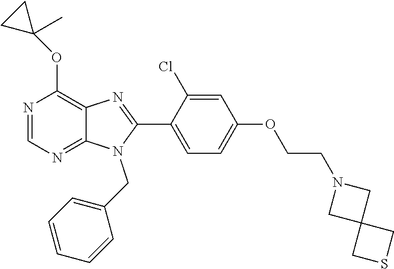 [SMILES: CC1(Oc2ncnc3c2nc(-c2ccc(OCCN4CC5(CSC5)C4)cc2Cl)n3Cc2ccccc2)CC1] | 6-(2-(4-(9- benzyl-6-(1- methylcyclo- propoxy)-9H- purin-8-yl)-3- chlorophenoxy) ethyl)-2-thia-6- azaspiro[3.3] heptane | (400 MHz, DMSO-d₆): 0.85 (2H, t), 1.03 (2H, t), 1.73 (3H, s), 2.70 (2H, t), 3.23-3.29 (8H, m), 4.03 (2H, t), 5.29 (2H, s), 6.87- 6.93 (2H, m), 6.98-7.03 (1H, m), 7.17-7.24 (4H, m), 7.37-7.44 (1H, m), 8.61 (1H, s) | 548 |

[H1] was prepared as follows:

2,6-Difluoro-4-(2-(4-methylpiperazin-1-yl)ethoxy)benzaldehyde (126 mg, 0.44 mmol) was added to N4-benzyl-6-(1-methylcyclopropoxy)pyrimidine-4,5-diamine (100 mg, 0.37 mmol, Synthetic Example 4 Intermediate) in DMSO (1 mL). The resulting mixture was stirred at 120° C. for 2 days. The solvent was removed under reduced pressure. The crude product was purified by preparative HPLC; Column: XBridge Prep OBD C18 Column, 30*150 mm, 5 μm, using decreasingly polar mixtures of water (containing 10 mmol/L NH₄HCO₃ and 0.1% conc. aqueous ammonia) and MeCN. Fractions containing the desired compound were evaporated to dryness to afford 9-benzyl-8-(2,6-difluoro-4-(2-(4-methylpiperazin-1-yl)ethoxy)phenyl)-6-(1-methylcyclopropoxy)-9H-purine (27 mg, 14%) as a red solid.

2,6-Difluoro-4-(2-(4-methylpiperazin-1-yl)ethoxy)benzaldehyde used as starting material was made as follows:

Tert-butyl 4-(2-(3,5-difluoro-4-formylphenoxy)ethyl)piperazine-1-carboxylate was made from a similar procedure as tert-butyl 4-(2-(3-chloro-4-formylphenoxy)ethyl)piperazine-1-carboxylate (Synthetic Example 4, intermediate), using 2,6-difluoro-4-hydroxybenzaldehyde and tert-butyl 4-(2-chloroethyl)piperazine-1-carboxylate as starting materials. ¹H NMR (400 MHz, DMSO-d₆): 1.40 (9H, s), 2.43 (4H, t), 2.73 (2H, t), 4.23 (2H, t), 6.91 (2H, d), 10.07 (1H, s). 4 protons not observed. m/z: ES+[M+H]+ 371.

2,6-Difluoro-4-(2-(4-methylpiperazin-1-yl)ethoxy)benzaldehyde was made from a similar procedure as 2-chloro-4-(2-(4-methylpiperazin-1-yl)ethoxy)benzaldehyde (Synthetic Example 4, intermediate), using tert-butyl 4-(2-(3,5-difluoro-4-formylphenoxy)ethyl)piperazine-1-carboxylate as starting material. ¹H NMR (300 MHz, DMSO-d₆): 2.12 (3H, s), 2.28 (4H, s), 2.43 (4H, s), 2.66 (2H, t), 4.19 (2H, t), 6.85-6.93 (2H, m), 10.05 (1H, s). m/z: ES+[M+H]+ 285.

[H2] made from a similar procedure as 9-benzyl-8-(2,6-difluoro-4-(2-(4-methylpiperazin-1-yl)ethoxy)phenyl)-6-(1-methylcyclopropoxy)-9H-purine (Synthetic Example H1), using N4-benzyl-6-(1-methylcyclopropoxy)pyrimidine-4,5-diamine (Synthetic Example 4, intermediate) and 2-fluoro-4-(2-(4-methylpiperazin-1-yl)ethoxy)benzaldehyde as starting materials.

2-Fluoro-4-(2-(4-methylpiperazin-1-yl)ethoxy)benzaldehyde made from a similar procedure as 2-chloro-4-(2-(4-methylpiperazin-1-yl)ethoxy)benzaldehyde (Synthetic Example 4, intermediate), using tert-butyl 4-(2-(3-fluoro-4-formylphenoxy)ethyl)piperazine-1-carboxylate as starting material. ¹H NMR (300 MHz, DMSO-d₆): 2.12 (3H, s), 2.29 (4H, s), 2.47 (4H, s), 2.68 (2H, t), 4.18 (2H, t), 6.93 (1H, m), 7.00 (1H, m), 7.75 (1H, t), 10.05 (1H, s). m/z: ES+[M+H]+ 267.

Tert-butyl 4-(2-(3-fluoro-4-formylphenoxy)ethyl)piperazine-1-carboxylate was made from a similar procedure as tert-butyl 4-(2-(3-chloro-4-formylphenoxy)ethyl)piperazine-1-carboxylate (Synthetic Example 4, intermediate), using 2-fluoro-4-hydroxybenzaldehyde and tert-butyl 4-(2-chloroethyl)piperazine-1-carboxylate as starting materials. ¹H NMR (400 MHz, DMSO-d₆): 1.40 (9H, s), 2.44 (4H, t), 2.74 (2H, t), 4.23 (2H, t), 6.96 (1H, m), 7.03 (1H, m), 7.78 (1H, t), 10.08 (1H, s). 4 protons not observed. m/z: ES+[M+H]+ 353.

[H3] made from a similar procedure as 9-benzyl-8-(2,6-difluoro-4-(2-(4-methylpiperazin-1-yl)ethoxy)phenyl)-6-(1-methylcyclopropoxy)-9H-purine (Synthetic Example H1) using N4-benzyl-6-(1-methylcyclopropoxy)pyrimidine-4,5-diamine (Synthetic Example 4, intermediate) and 2-methoxy-4-(2-(4-methylpiperazin-1-yl)ethoxy)benzaldehyde as starting materials.

2-Methoxy-4-(2-(4-methylpiperazin-1-yl)ethoxy)benzaldehyde made from a similar procedure as 2,6-difluoro-4-(2-(4-methylpiperazin-1-yl)ethoxy)benzaldehyde (Synthetic Example H1, intermediate), using tert-butyl 4-(2-(4-formyl-3-methoxyphenoxy)ethyl)piperazine-1-carboxylate as starting material. ¹H NMR (300 MHz, DMSO-d₆): 2.13 (3H, s), 2.30 (4H, s), 2.47 (4H, s), 2.68 (2H, t), 3.89 (3H, s), 4.18 (2H, t), 6.63 (1H, m), 6.68 (1H, d), 7.63 (1H, d), 10.16 (1H, s). m/z: ES+[M+H]+ 279.

Tert-butyl 4-(2-(4-formyl-3-methoxyphenoxy)ethyl)piperazine-1-carboxylate was made from a similar procedure as tert-butyl 4-(2-(3,5-difluoro-4-formylphenoxy)ethyl)piperazine-1-carboxylate (Synthetic Example H1, intermediate), using 4-hydroxy-2-methoxybenzaldehyde and tert-butyl 4-(2-chloroethyl)piperazine-1-carboxylate as starting materials. ¹H NMR (400 MHz, DMSO-d₆): 1.40 (9H, s), 2.45 (4H, t), 2.74 (2H, t), 3.91 (3H, s), 4.22 (2H, t), 6.64-6.68 (1H, m), 6.70 (1H, d), 7.65 (1H, d), 10.18 (1H, s). 4 protons not observed. m/z: ES+[M+H]+ 365.

[H4] made from a similar procedure as 9-benzyl-8-(2,6-difluoro-4-(2-(4-methylpiperazin-1-yl)ethoxy)phenyl)-6-(1-methylcyclopropoxy)-9H-purine (Synthetic Example H1) using N4-benzyl-6-(1-methylcyclopropoxy)pyrimidine-4,5-diamine (Synthetic Example 4, intermediate) and 2,6-dimethyl-4-(2-(4-methylpiperazin-1-yl)ethoxy)benzaldehyde as starting materials.

2,6-Dimethyl-4-(2-(4-methylpiperazin-1-yl)ethoxy)benzaldehyde was made from a similar procedure as 2,6-difluoro-4-(2-(4-methylpiperazin-1-yl)ethoxy)benzaldehyde (Synthetic Example H1, intermediate), using tert-butyl 4-(2-(4-formyl-3,5-dimethylphenoxy)ethyl)piperazine-1-carboxylate as starting material. ¹H NMR (300 MHz, DMSO-d₆): 2.12 (3H, s), 2.29 (4H, s), 2.44 (4H, s), 2.52 (6H, s), 2.66 (2H, t), 4.12 (2H, t), 6.72 (2H, s), 10.35 (1H, s). m/z: ES+[M+H]+ 277.

Tert-butyl 4-(2-(4-formyl-3,5-dimethylphenoxy)ethyl)piperazine-1-carboxylate was made from a similar procedure as tert-butyl 4-(2-(3,5-difluoro-4-formylphenoxy)ethyl)piperazine-1-carboxylate (Synthetic Example H1, intermediate), using 4-hydroxy-2,6-dimethylbenzaldehyde and tert-butyl 4-(2-chloroethyl)piperazine-1-carboxylate as starting materials. ¹H NMR (400 MHz, DMSO-d₆): 1.40 (9H, s), 2.44 (4H, t), 2.55 (6H, s), 2.72 (2H, t), 4.17 (2H, t), 6.74 (2H, s), 10.38 (1H, s). 4 protons not observed. m/z: ES+[M+H]+ 363.

[H5] made from a similar procedure as 9-benzyl-8-(2,6-difluoro-4-(2-(4-methylpiperazin-1-yl)ethoxy)phenyl)-6-(1-methylcyclopropoxy)-9H-purine (Synthetic Example H1) using N4-benzyl-6-(1-methylcyclopropoxy)pyrimidine-4,5-diamine (Synthetic Example 4, intermediate) and 2,3-difluoro-4-(2-(4-methylpiperazin-1-yl)ethoxy)benzaldehyde as starting materials.

2,3-Difluoro-4-(2-(4-methylpiperazin-1-yl)ethoxy)benzaldehyde made from a similar procedure as 2,6-difluoro-4-(2-(4-methylpiperazin-1-yl)ethoxy)benzaldehyde (Synthetic Example H1, intermediate), using tert-butyl 4-(2-(2,3-difluoro-4-formylphenoxy)ethyl)piperazine-1-carboxylate as starting material. ¹H NMR (300 MHz, DMSO-d₆): 2.14 (3H, s), 2.30 (4H, s), 2.50 (4H, s), 2.74 (2H, t), 4.32 (2H, t), 7.26 (1H, m), 7.67 (1H, m), 10.05 (1H, s). m/z: ES+[M+H]+ 285.

Tert-butyl 4-(2-(2,3-difluoro-4-formylphenoxy)ethyl)piperazine-1-carboxylate was made from a similar procedure as tert-butyl 4-(2-(3,5-difluoro-4-formylphenoxy)ethyl)piperazine-1-carboxylate (Synthetic Example H1, intermediate), using 2,3-difluoro-4-hydroxybenzaldehyde and tert-butyl 4-(2-chloroethyl)piperazine-1-carboxylate as starting materials. ¹H NMR (400 MHz, DMSO-d₆): 1.39 (9H, s), 2.44 (4H, s), 2.78 (2H, m), 4.34 (2H, m), 7.26 (1H, t), 7.67 (1H, t), 10.04 (1H, s). 4 protons not observed. m/z: ES+[M+H]+ 371.

[H6] made from a similar procedure as 9-benzyl-8-(2,6-difluoro-4-(2-(4-methylpiperazin-1-yl)ethoxy)phenyl)-6-(1-methylcyclopropoxy)-9H-purine (Synthetic Example H1) using N4-benzyl-6-(1-methylcyclopropoxy)pyrimidine-4,5-diamine (Synthetic Example 4, intermediate) and 2-bromo-4-(2-(4-methylpiperazin-1-yl)ethoxy)benzaldehyde as starting materials.

2-Bromo-4-(2-(4-methylpiperazin-1-yl)ethoxy)benzaldehyde made from a similar procedure as 2,6-difluoro-4-(2-(4-methylpiperazin-1-yl)ethoxy)benzaldehyde (Synthetic Example H1, intermediate), using tert-butyl 4-(2-(3-bromo-4-formylphenoxy)ethyl)piperazine-1-carboxylate as starting material. ¹H NMR (300 MHz, DMSO-d₆): 2.12 (3H, s), 2.29 (4H, s), 2.45 (4H, s), 2.67 (2H, t), 4.20 (2H, t), 7.11 (1H, m), 7.36 (1H, d), 7.79 (1H, d), 10.07 (1H, s). m/z: ES+[M+H]+ 327.

Tert-butyl 4-(2-(3-bromo-4-formylphenoxy)ethyl)piperazine-1-carboxylate was made from a similar procedure as tert-butyl 4-(2-(3,5-difluoro-4-formylphenoxy)ethyl)piperazine-1-carboxylate (Synthetic Example H1, intermediate), using 2-bromo-4-hydroxybenzaldehyde and tert-butyl 4-(2-chloroethyl)piperazine-1-carboxylate as starting materials ¹H NMR (400 MHz, DMSO-d₆): 1.40 (9H, s), 2.44 (4H, t), 2.74 (2H, t), 4.24 (2H, t), 7.14 (1H, m), 7.38 (1H, d), 7.81 (1H, d), 10.09 (1H, m). 4 protons not observed. m/z: ES+[M+H]+ 413.

[H7] was made following a similar procedure to 4-((8-(2-chloro-4-(2-(4-methylpiperazin-1-yl)ethoxy)phenyl)-6-(1-methylcyclopropoxy)-9H-purin-9-yl)methyl)-2-methylthiazole (Synthetic Example H1), using 6-(1-methylcyclopropoxy)-5-nitro-N-(pyridin-2-ylmethyl)pyrimidin-4-amine (Synthetic Example E5, intermediate) and 2-chloro-4-(2-(4-methylpiperazin-1-yl)ethoxy)benzaldehyde (Synthetic Example 4, intermediate) as starting materials.

[H8] was synthesized as follows:

Sodium triacetoxyborohydride (46.6 mg, 0.22 mmol) was added to 2-(9-benzyl-6-(1-methylcyclopropoxy)-9H-purin-8-yl)-5-(2-(piperazin-1-yl)ethoxy)benzonitrile, hydrochloride salt (30 mg, 0.05 mmol) in 37% aqueous formaldehyde solution (1 mL). The resulting mixture was stirred at room temperature for 1 hour. The solvent was removed under reduced pressure. The crude product was purified by preparative HPLC (XBridge Prep OBD C18 column, 30*150 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness to afford 2-(9-benzyl-6-(1-methylcyclopropoxy)-9H-purin-8-yl)-5-(2-(4-methylpiperazin-1-yl)ethoxy)benzonitrile (12.0 mg, 42%) as a light yellow solid.

2-(9-Benzyl-6-(1-methylcyclopropoxy)-9H-purin-8-yl)-5-(2-(piperazin-1-yl)ethoxy)benzonitrile, hydrochloride salt was made from a similar procedure as Synthetic Example 16, using tert-butyl 4-(2-(4-(9-benzyl-6-(1-methylcyclopropoxy)-9H-purin-8-yl)-3-cyanophenoxy)ethyl)piperazine-1-carboxylate as starting material and EtOAc as solvent. The crude product was used in next step without further purification. m/z: ES+[M+H]+ 510.

Tert-butyl 4-(2-(4-(9-benzyl-6-(1-methylcyclopropoxy)-9H-purin-8-yl)-3-cyanophenoxy)ethyl)piperazine-1-carboxylate was made from a similar procedure as Synthetic Example H1 using N4-benzyl-6-(1-methylcyclopropoxy)pyrimidine-4,5-diamine (Synthetic Example 4, intermediate) and tert-butyl 4-(2-(3-cyano-4-formylphenoxy)ethyl)piperazine-1-carboxylate as starting materials. ¹H NMR (400 MHz, DMSO-d₆): 0.87 (2H, d), 1.04 (2H, d), 1.40 (9H, s), 1.75 (3H, s), 2.45 (4H, t), 2.75 (2H, t), 3.30 (4H, s), 4.24 (2H, t), 5.44 (2H, s), 6.89 (2H, dd), 7.17-7.25 (3H, m), 7.41 (1H, dd), 7.57-7.79 (2H, m), 8.66 (1H, s). m/z: ES+[M+H]+ 610.

Tert-butyl 4-(2-(3-cyano-4-formylphenoxy)ethyl)piperazine-1-carboxylate was made from a similar procedure as tert-butyl 4-(2-(3,5-difluoro-4-formylphenoxy)ethyl)piperazine-1-carboxylate (Synthetic Example H1, intermediate), using 2-formyl-5-hydroxybenzonitrile and tert-butyl 4-(2-chloroethyl)piperazine-1-carboxylate as starting materials ¹H NMR (400 MHz, DMSO-d₆): 1.40 (9H, s), 2.45 (4H, d), 2.76 (2H, t), 3.31 (4H, s), 4.29 (2H, t), 7.48 (1H, dd), 7.66 (1H, d), 8.04 (1H, d), 10.01 (1H, s). m/z: ES+[M+H]+ 360.

[H9] was synthesized as follows:

TBAF (353 μL, 0.35 mmol, 1M in THF) was added to 9-benzyl-6-(1-methylcyclopropoxy)-8-(4-(2-(4-methylpiperazin-1-yl)ethoxy)-2-((triisopropylsilyl)ethynyl)phenyl)-9H-purine (200 mg, 0.29 mmol) in THF (3 mL) at room temperature. The resulting mixture was stirred at room temperature for 1 hour. The reaction mixture was concentrated and the resulting crude product was purified by preparative HPLC (XBridge Prep OBD C18 column, 30*150 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness to afford 9-benzyl-8-(2-ethynyl-4-(2-(4-methylpiperazin-1-yl)ethoxy)phenyl)-6-(1-methylcyclopropoxy)-9H-purine (0.024 g, 15%) as a brown solid.

9-Benzyl-6-(1-methylcyclopropoxy)-8-(4-(2-(4-methylpiperazin-1-yl)ethoxy)-2-((triisopropylsilyl)ethynyl)phenyl)-9H-purine was made from a similar procedure as Synthetic Example H1 using N4-benzyl-6-(1-methylcyclopropoxy)pyrimidine-4,5-diamine (Synthetic Example 4, intermediate) and 4-(2-(4-methylpiperazin-1-yl)ethoxy)-2-((triisopropylsilyl)ethynyl)benzaldehyde as starting materials. m/z: ES+[M+H]+ 679.

4-(2-(4-Methylpiperazin-1-yl)ethoxy)-2-((triisopropylsilyl)ethynyl)benzaldehyde was synthesized as follows:

Bis(triphenylphosphine)palladium(II) dichloride (644 mg, 0.92 mmol) was added to 2-bromo-4-(2-(4-methylpiperazin-1-yl)ethoxy)benzaldehyde (Synthetic Example H6 intermediate, 300 mg, 0.92 mmol), cuprous iodide (175 mg, 0.92 mmol) and (triisopropylsilyl)acetylene (334 mg, 1.83 mmol) in TEA (4 mL) at room temperature. The resulting mixture was stirred at room temperature for 1 hour. The reaction mixture was evaporated to dryness and redissolved in EtOAc (50 mL), and washed sequentially with water (50 mL×3) and saturated brine (50 mL x1). The organic layer was dried over Na₂SO₄, filtered and evaporated to afford crude product. The crude product was purified by flash silica chromatography, elution gradient 0 to 50% EtOAc in petroleum ether. Pure fractions were evaporated to dryness to afford 4-(2-(4-methylpiperazin-1-yl)ethoxy)-2-((triisopropylsilyl)ethynyl)benzaldehyde (0.300 g, 76%) as a pale yellow oil. ¹H NMR (400 MHz, DMSO-d₆): 1.11 (21H, s), 2.16 (3H, s), 2.34 (5H, s), 2.51 (3H, s), 2.69 (2H, s), 4.22 (2H, s), 7.14 (2H, s), 7.78 (1H, s), 10.31 (1H, s). m/z: ES+[M+H]+ 429.

[H10] was synthesized as follows:

LiAlH₄ (2.4 M in THF, 0.037 mL, 0.09 mmol) was added to methyl 2-(9-benzyl-6-(1-methylcyclopropoxy)-9H-purin-8-yl)-5-(2-(4-methylpiperazin-1-yl)ethoxy)benzoate (0.1 g, 0.18 mmol) in THF (3 mL) at 0° C. The resulting mixture was stirred at room temperature for 1 hour. The reaction mixture was quenched with water (5 mL) and the solvent removed under reduced pressure. The crude product was purified by preparative HPLC (XBridge Prep OBD C18 column, 30*150 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness to afford (2-(9-benzyl-6-(1-methylcyclopropoxy)-9H-purin-8-yl)-5-(2-(4-methylpiperazin-1-yl)ethoxy)phenyl)methanol (0.031 g, 32%) as a white solid.

Methyl 2-(9-benzyl-6-(1-methylcyclopropoxy)-9H-purin-8-yl)-5-(2-(4-methylpiperazin-1-yl)ethoxy)benzoate was synthesized as follows:

CH₃OH (5.0 mL) was added to 2-(9-benzyl-6-(1-methylcyclopropoxy)-9H-purin-8-yl)-5-(2-(4-methylpiperazin-1-yl)ethoxy)benzoic acid (700 mg, 1.29 mmol), HATU (736 mg, 1.93 mmol) and DIEA (676 μL, 3.87 mmol) in DMA (5 mL) at room temperature. The resulting mixture was stirred at room temperature for 1 hour. The reaction mixture was evaporated, EtOAc (100 mL) was added, and the organic layer washed sequentially with water (100 mL×3) and saturated brine (100 mL x1). The organic layer was dried over Na₂SO₄, filtered and evaporated to afford crude product. The crude product was purified by flash silica chromatography, elution gradient 0 to 10% MeOH in DCM. Pure fractions were evaporated to dryness to afford methyl 2-(9-benzyl-6-(1-methylcyclopropoxy)-9H-purin-8-yl)-5-(2-(4-methylpiperazin-1-yl)ethoxy)benzoate (0.600 g, 84%) as a brown gum. m/z: ES+[M+H]+ 557.

2-(9-Benzyl-6-(1-methylcyclopropoxy)-9H-purin-8-yl)-5-(2-(4-methylpiperazin-1-yl)ethoxy)benzoic acid was made from a similar procedure as Synthetic Example H1 using N4-benzyl-6-(1-methylcyclopropoxy)pyrimidine-4,5-diamine (Synthetic Example 4, intermediate) and methyl 2-formyl-5-(2-(4-methylpiperazin-1-yl)ethoxy)benzoate as starting materials. m/z: ES+[M+H]+ 543.

Methyl 2-formyl-5-(2-(4-methylpiperazin-1-yl)ethoxy)benzoate was synthesized as follows:

PdCl₂(dppf) (335 mg, 0.46 mmol), 2-bromo-4-(2-(4-methylpiperazin-1-yl)ethoxy)benzaldehyde (300 mg, 0.92 mmol; Synthetic Example H6, intermediate) and TEA (383 μL, 2.75 mmol) in CH30H (5 mL) were stirred under an atmosphere of carbon monoxide at 10 atm and 100° C. for 16 hours. The reaction mixture was concentrated and diluted with EtOAc (50 mL), and washed sequentially with water (50 mL×3) and saturated brine (50 mL×1). The organic layer was dried over Na₂SO₄, filtered and evaporated to afford the crude product. The crude product was purified by flash silica chromatography, elution gradient 0 to 10% MeOH in DCM. Pure fractions were evaporated to dryness to afford methyl 2-formyl-5-(2-(4-methylpiperazin-1-yl)ethoxy)benzoate (0.200 g, 71%) as a brown gum. m/z: ES+[M+H]+ 307.

[H11] was obtained from the cyclisation of 3-(3-chloro-4-formylphenoxy)propanoic acid and N4-benzyl-6-(1-methylcyclopropoxy)pyrimidine-4,5-diamine (Synthetic Example 4, intermediate synthesis), following the procedure described for the synthesis of 9-benzyl-8-(2,6-difluoro-4-(2-(4-methylpiperazin-1-yl)ethoxy)phenyl)-6-(1-methylcyclopropoxy)-9H-purine (Synthetic Example H1).

3-(3-Chloro-4-formylphenoxy)propanoic acid was made through a Williamson ether synthesis of 3-bromopropanoic acid and 2-chloro-4-hydroxybenzaldehyde as follows:

2-chloro-4-hydroxybenzaldehyde (1.0 g, 6.39 mmol), 3-bromopropanoic acid (1.47 g, 9.58 mmol) and K₂CO₃ (2.65 g, 19.2 mmol) in MeCN (10 mL) were stirred at 80° C. for 12 hours. The solvent was removed under reduced pressure. The crude product was purified by flash C18-flash chromatography, elution gradient 5 to 100% MeCN in water (0.05% formic acid). Pure fractions were evaporated to dryness to afford 3-(3-chloro-4-formylphenoxy)propanoic acid (0.40 g, 27%) as a yellow solid. ¹H NMR (300 MHz, DMSO-d₆): 2.72 (2H, t), 4.30 (2H, t), 7.08 (1H, dd), 7.19 (1H, d), 7.81 (1H, d), 10.18 (1H, d), 12.44 (1H, s). m/z: ES−[M−H]−227.

[H12] was obtained from the cyclisation of (2-(3-chloro-4-formylphenoxy)ethyl)-L-proline and N4-benzyl-6-(1-methylcyclopropoxy)pyrimidine-4,5-diamine (Synthetic Example 4, intermediate) in a similar procedure to Synthetic Example P2.

(2-(3-Chloro-4-formylphenoxy)ethyl)-L-proline was made from methyl (2-(3-chloro-4-formylphenoxy)ethyl)-L-prolinate through an ester hydrolysis reaction similar to the reaction used to make (E)-3-(4-(9-benzyl-6-(1-methylcyclopropoxy)-9H-purin-8-yl)-3-chlorophenyl)acrylic acid (Synthetic Example M4). ¹H NMR (400 MHz, DMSO-d₆): 1.5-1.79 (3H, m), 1.88-2.07 (1H, m), 2.5-2.64 (1H, m), 2.88-3.01 (1H, m), 3.04-3.2 (2H, m), 3.22-3.34 (1H, m), 4.16 (2H, t), 6.96 (1H, dd), 7.07 (1H, d), 7.72 (1H, d), 10.08 (1H, s). One proton not observed. m/z: ES+[M+H]+ 298.

Methyl (2-(3-chloro-4-formylphenoxy)ethyl)-L-prolinate was made by N-alkylation using a similar procedure used in the synthesis of (S)-9-benzyl-8-(2-chloro-4-(2-(3-methylpiperazin-1-yl)ethoxy)phenyl)-6-(1-methylcyclopropoxy)-9H-purine (Synthetic Example R3), using methyl L-prolinate hydrochloride and 4-(2-bromoethoxy)-2-chlorobenzaldehyde (Synthetic Example 18, intermediate) as starting material. ¹H NMR (300 MHz, DMSO-d₆): 1.79-1.99 (1H, m), 1.99-2.1 (2H, m), 2.36-2.48 (1H, m), 3.23-3.5 (1H, m), 3.58-3.78 (7H, m), 4.46-4.54 (2H, m), 7.11 (1H, dd), 7.24 (1H, d), 7.89 (1H, d), 10.23 (1H, s). m/z: ES+[M+H]+ 312.

[H13] was obtained from the cyclisation of (R)-2-chloro-4-(2-(3-hydroxypyrrolidin-1-yl)ethoxy)benzaldehyde and N4-benzyl-6-(1-methylcyclopropoxy)pyrimidine-4,5-diamine (Synthetic Example 4, intermediate) in a similar procedure to Synthetic Example P2.

(R)-2-Chloro-4-(2-(3-hydroxypyrrolidin-1-yl)ethoxy)benzaldehyde was made by N-alkylation using a similar procedure used in the synthesis of (S)-9-benzyl-8-(2-chloro-4-(2-(3-methylpiperazin-1-yl)ethoxy)phenyl)-6-(1-methylcyclopropoxy)-9H-purine (Synthetic Example R3), using (R)-pyrrolidin-3-ol and 4-(2-bromoethoxy)-2-chlorobenzaldehyde (Synthetic Example 18, intermediate) as starting material. ¹H NMR (400 MHz, DMSO-d₆): 1.49-1.57 (1H, m), 1.90-2.01 (1H, m), 2.38-2.42 (1H, m), 2.61-2.70 (1H, m), 2.74-2.84 (3H, m), 3.17 (1H, s), 4.14-4.24 (3H, m), 4.71 (1H, s), 7.09 (1H, dd), 7.19 (1H, d), 7.82 (1H, d), 10.19 (1H, s). m/z: ES+[M+H]+ 270.

[H14] was obtained from the cyclisation of 2-chloro-4-(2-(3-hydroxyazetidin-1-yl)ethoxy)benzaldehyde and N4-benzyl-6-(1-methylcyclopropoxy)pyrimidine-4,5-diamine (Synthetic Example 4, intermediate) in a similar procedure to Synthetic Example P2.

2-Chloro-4-(2-(3-hydroxyazetidin-1-yl)ethoxy)benzaldehyde was made by N-alkylation using a similar procedure used in the synthesis of (S)-9-benzyl-8-(2-chloro-4-(2-(3-methylpiperazin-1-yl)ethoxy)phenyl)-6-(1-methylcyclopropoxy)-9H-purine (Synthetic Example R3), using azetidin-3-ol and 4-(2-bromoethoxy)-2-chlorobenzaldehyde (Synthetic Example 18, intermediate) as starting material. ¹H NMR (400 MHz, DMSO-d₆): 2.80-2.94 (4H, m), 3.61-3.65 (2H, m), 4.10 (2H, t), 4.16-4.20 (1H, m), 7.02-7.11 (1H, m), 7.17 (1H, d), 7.82 (1H, d), 10.19 (1H, s). One proton not observed. m/z: ES+[M+H]+ 256.

[H15] was obtained from the cyclisation of 2-chloro-4-(2-(4-hydroxypiperidin-1-yl)ethoxy)benzaldehyde and N4-benzyl-6-(1-methylcyclopropoxy)pyrimidine-4,5-diamine (Synthetic Example 4, intermediate) in a similar procedure to Synthetic Example P2.

2-Chloro-4-(2-(4-hydroxypiperidin-1-yl)ethoxy)benzaldehyde was made by N-alkylation using a similar procedure used in the synthesis of (R)-1-(2-(3-Chloro-4-(9-((4-chloropyridin-2-yl)methyl)-6-(1-methylcyclopropoxy)-9H-purin-8-yl)phenoxy)ethyl)pyrrolidin-3-ol (Synthetic Example R2), using piperidin-4-ol and 4-(2-bromoethoxy)-2-chlorobenzaldehyde (Synthetic Example 18, intermediate) as starting material. ¹H NMR (300 MHz, DMSO-d₆): 1.35-1.92 (4H, m), 2.02-2.14 (2H, m), 3.02-3.2 (2H, m), 3.5-3.7 (2H, m), 4.28-4.32 (2H, m), 7.11 (1H, d), 7.23 (1H, s), 7.83 (1H, d), 8.19 (1H, s), 10.19 (1H, s). One proton not observed. m/z: ES+[M+H]+ 284.

[H16] was obtained from the cyclisation of 2-chloro-4-(2-(6-hydroxy-2-azaspiro[3.3]heptan-2-yl)ethoxy)benzaldehyde and N4-benzyl-6-(1-methylcyclopropoxy)pyrimidine-4,5-diamine (Synthetic Example 4, intermediate) in a similar procedure to Synthetic Example P2.

2-Chloro-4-(2-(6-hydroxy-2-azaspiro[3.3]heptan-2-yl)ethoxy)benzaldehyde was made by N-alkylation using a similar procedure used in the synthesis of (S)-9-benzyl-8-(2-chloro-4-(2-(3-methylpiperazin-1-yl)ethoxy)phenyl)-6-(1-methylcyclopropoxy)-9H-purin (Synthetic Example R3), using 2-azaspiro[3.3]heptan-6-ol and 4-(2-bromoethoxy)-2-chlorobenzaldehyde (Synthetic Example 18, intermediate) as starting material. ¹H NMR (400 MHz, DMSO-d₆): 1.80-1.93 (2H, m), 2.28-2.37 (2H, m), 2.78-2.82 (2H, m), 3.17-3.30 (4H, m), 3.88-3.96 (1H, m), 4.08 (2H, t), 4.95 (1H, d), 7.06-7.12 (1H, m), 7.14-7.20 (1H, m), 7.79-7.83 (1H, m), 10.19 (1H, s). m/z: ES+[M+H]+ 296.

[H17] was obtained from the cyclisation of 2-chloro-4-(3-(3-hydroxyazetidin-1-yl)propoxy)benzaldehyde and N4-benzyl-6-(1-methylcyclopropoxy)pyrimidine-4,5-diamine (Synthetic Example 4, intermediate) in a similar procedure to Synthetic Example P2.

2-Chloro-4-(3-(3-hydroxyazetidin-1-yl)propoxy)benzaldehyde was made in a N-alkylation using a similar procedure use in the synthesis of (S)-9-benzyl-8-(2-chloro-4-(2-(3-methylpiperazin-1-yl)ethoxy)phenyl)-6-(1-methylcyclopropoxy)-9H-purine (Synthetic Example R3), using azetidin-3-ol and 4-(3-bromopropoxy)-2-chlorobenzaldehyde as starting material. ¹H NMR (400 MHz, DMSO-d₆): 1.68-1.81 (2H, m), 2.62 (2H, t), 2.77-2.99 (2H, m), 3.5-3.65 (2H, m), 3.99-4.28 (4H, m), 7.01-7.11 (1H, m), 7.14-7.23 (1H, m), 7.79-7.83 (1H, m), 10.19 (1H, s). m/z: ES+[M+H]+ 270.

4-(3-Bromopropoxy)-2-chlorobenzaldehyde was formed in a similar manner to 4-(2-bromoethoxy)-2-chlorobenzaldehyde (Synthetic Example 18, intermediate) using 1,3-dibromopropane and 2-chloro-4-hydroxybenzaldehyde as starting material. ¹H NMR (400 MHz, DMSO-d₆): 2.18-2.33 (2H, m), 3.65 (2H, t), 4.20 (2H, t), 7.08 (1H, dd), 7.17 (1H, d), 7.81 (1H, d), 10.17 (1H, s). m/z: ES+[M+H]+ 276.9.

[H18] was obtained from tert-butyl 6-(2-(3-chloro-4-formylphenoxy)ethyl)-1,6-diazaspiro[3.3]heptane-1-carboxylate via a BOC deprotection similar to the procedure described in Synthetic Example R3 in the synthesis of (S)-9-benzyl-8-(2-chloro-4-(2-(3-methylpiperazin-1-yl)ethoxy)phenyl)-6-(1-methylcyclopropoxy)-9H-purine.

Tert-butyl 6-(2-(4-(9-benzyl-6-(1-methylcyclopropoxy)-9H-purin-8-yl)-3-chlorophenoxy)ethyl)-1,6-diazaspiro[3.3]heptane-1-carboxylate was made by cyclisation of tert-butyl 6-(2-(3-chloro-4-formylphenoxy)ethyl)-1,6-diazaspiro[3.3]heptane-1-carboxylate and N4-benzyl-6-(1-methylcyclopropoxy)pyrimidine-4,5-diamine (Synthetic Example 4, intermediate) in a similar procedure to Synthetic Example P2. ¹H NMR (400 MHz, DMSO-d₆): 0.78-0.86 (2H, m), 0.97-1.04 (2H, m), 1.41 (9H, s), 1.73 (3H, s), 2.36-2.4 (2H, m), 2.77-2.82 (2H, m), 3.42-3.45 (2H, m), 3.59-3.69 (4H, m), 4.02-4.08 (2H, m), 5.28 (2H, s), 6.82-6.94 (2H, m), 6.94-7.03 (1H, m), 7.17-7.24 (4H, m), 7.27-7.33 (1H, m), 8.61 (1H, s). m/z: ES+[M+H]+ 631.

Tert-butyl 6-(2-(3-chloro-4-formylphenoxy)ethyl)-1,6-diazaspiro[3.3]heptane-1-carboxylate was made by N-alkylation using a similar procedure used in the synthesis of (S)-9-benzyl-8-(2-chloro-4-(2-(3-methylpiperazin-1-yl)ethoxy)phenyl)-6-(1-methylcyclopropoxy)-9H-purine (Synthetic Example R3), using tert-butyl 1,6-diazaspiro[3.3]heptane-1-carboxylate and 4-(2-bromoethoxy)-2-chlorobenzaldehyde (Synthetic Example 18, intermediate) as starting material. ¹H NMR (400 MHz, DMSO-d₆): 1.40 (9H, s), 2.31-2.39 (2H, m), 2.74-2.87 (2H, m), 3.41-3.5 (2H, m), 3.5-3.77 (4H, m), 4.04-4.16 (2H, m), 7.07 (1H, dd), 7.18 (1H, d), 7.82 (1H, d), 10.19 (1H, s). m/z: ES+[M+H]+ 381.

[H19] was obtained from the cyclisation of (S)-2-chloro-4-(2-(2-methyl-3-oxopiperazin-1-yl)ethoxy)benzaldehyde and N4-benzyl-6-(1-methylcyclopropoxy)pyrimidine-4,5-diamine (Synthetic Example 4, intermediate) in a similar procedure to Synthetic Example P2.

(S)-2-chloro-4-(2-(2-methyl-3-oxopiperazin-1-yl)ethoxy)benzaldehyde was made by N-alkylation using a similar procedure used in the synthesis of (S)-9-benzyl-8-(2-chloro-4-(2-(3-methylpiperazin-1-yl)ethoxy)phenyl)-6-(1-methylcyclopropoxy)-9H-purine (Synthetic Example R3), using (S)-3-methylpiperazin-2-one and 4-(2-bromoethoxy)-2-chlorobenzaldehyde (Synthetic Example 18, intermediate) as starting material. ¹H NMR (300 MHz, DMSO-d₆): 1.22 (3H, d), 2.55-2.65 (1H, m), 2.72-2.84 (1H, m), 2.93-3.04 (2H, m), 3.13-3.18 (3H, m), 4.19-4.3 (2H, m), 7.07-7.13 (1H, m), 7.22 (1H, d), 7.67 (1H, s), 7.83 (1H, d), 10.20 (1H, d). m/z: ES+[M+H]+ 297.

[H20] was obtained from the oxidation of 6-(2-(4-(9-benzyl-6-(1-methylcyclopropoxy)-9H-purin-8-yl)-3-chlorophenoxy)ethyl)-2-thia-6-azaspiro[3.3]heptane (Synthetic Example H23) as follows:

Hydrogen peroxide (30% aqueous, 807 mg, 23.72 mmol) was added portionwise to a solution of 6-(2-(4-(9-benzyl-6-(1-methylcyclopropoxy)-9H-purin-8-yl)-3-chlorophenoxy)ethyl)-2-thia-6-azaspiro[3.3]heptane (130 mg, 0.24 mmol, Synthetic Example H23) in dry acetic acid (1.42 g, 23.72 mmol) at 0° C. for 3 hours. The reaction mixture was diluted with DCM (50 mL), and washed sequentially with saturated NaHCO₃ (100 mL), water (100 mL) and saturated brine (100 mL). The organic layer was dried over Na₂SO₄, filtered and evaporated to afford the crude product. The crude product was purified by preparative HPLC (YMC-Actus Triart C18 ExRS column, 30 mm×150 mm, 5 μm), using decreasingly polar mixtures of water (containing 10 mmol/L NH₄HCO₃ and 0.1% aqueous ammonia) and MeCN as eluents. The fractions containing the desired compound were evaporated to dryness to afford Synthetic Example H20 (4.0 mg, 3%) as a white solid.

6-(2-(4-(9-Benzyl-6-(1-methylcyclopropoxy)-9H-purin-8-yl)-3-chlorophenoxy)ethyl)-2-thia-6-azaspiro[3.3]heptane (Synthetic Example H23) was formed from the cyclisation of (E)-5-((4-(2-(2-thia-6-azaspiro[3.3]heptan-6-yl)ethoxy)-2-chlorobenzylidene)amino)-N-benzyl-6-(1-methylcyclopropoxy)pyrimidin-4-amine in a similar procedure to (E)-N-benzyl-5-((2-chloro-4-(2-(4-methylpiperazin-1-yl)ethoxy)benzylidene)amino)-6-(1-methylcyclopropoxy)pyrimidin-4-amine (Synthetic Example 4, intermediate).

(E)-5-((4-(2-(2-thia-6-azaspiro[3.3]heptan-6-yl)ethoxy)-2-chlorobenzylidene)amino)-N-benzyl-6-(1-methylcyclopropoxy)pyrimidin-4-amine was made using a similar method to tert-butyl (E)-4-(2-(3-chloro-4-(((4-((3-chlorobenzyl)amino)-6-(1-methylcyclopropoxy)pyrimidin-5-yl)imino)methyl)phenoxy)ethyl)piperazine-1-carboxylate (Synthetic Example J13, intermediate), using 4-(2-(2-thia-6-azaspiro[3.3]heptan-6-yl)ethoxy)-2-chlorobenzaldehyde and N4-benzyl-6-(1-methylcyclopropoxy)pyrimidine-4,5-diamine (Synthetic Example 4, intermediate). m/z: ES+[M+H]+ 550.

4-(2-(2-Thia-6-azaspiro[3.3]heptan-6-yl)ethoxy)-2-chlorobenzaldehyde was made by N-alkylation using a similar procedure use in the synthesis of (S)-9-benzyl-8-(2-chloro-4-(2-(3-methylpiperazin-1-yl)ethoxy)phenyl)-6-(1-methylcyclopropoxy)-9H-purine (Synthetic Example R3), using 2-thia-6-azaspiro[3.3]heptane, hemioxalate salt and 4-(2-bromoethoxy)-2-chlorobenzaldehyde (Synthetic Example 18, intermediate) as starting material. ¹H NMR (300 MHz, DMSO-d₆): 2.70 (2H, t), 3.23-3.27 (8H, m), 4.07 (2H, t), 7.06 (1H, dd), 7.17 (1H, d), 7.82 (1H, d), 10.19 (1H, s). m/z: ES+[M+H]+ 298.

[H21] was obtained from the reaction of 2-chloro-4-(2-(5-oxo-1,4-diazepan-1-yl)ethoxy)benzaldehyde and N4-benzyl-6-(1-methylcyclopropoxy)pyrimidine-4,5-diamine (Synthetic Example 4, intermediate) in a similar procedure to Synthetic Example P2. 2-Chloro-4-(2-(5-oxo-1,4-diazepan-1-yl)ethoxy)benzaldehyde was made by N-alkylation using a similar procedure used in the synthesis of (S)-9-benzyl-8-(2-chloro-4-(2-(3-methylpiperazin-1-yl)ethoxy)phenyl)-6-(1-methylcyclopropoxy)-9H-purine (Synthetic Example R3), using 1,4-diazepan-5-one and 4-(2-bromoethoxy)-2-chlorobenzaldehyde (Synthetic Example 18, intermediate) as starting material. ¹H NMR (300 MHz, DMSO-d₆): 2.39-2.46 (2H, m), 2.59-2.71 (4H, m), 2.84-2.92 (2H, m), 3.12 (2H, s), 4.24 (2H, t), 7.05-7.13 (1H, m), 7.22 (1H, d), 7.54 (1H, s), 7.83 (1H, d), 10.20 (1H, d). m/z: ES+[M+H]+ 297.

[H22] was obtained from the cyclisation of (R)-2-chloro-4-(2-(2-methyl-3-oxopiperazin-1-yl)ethoxy)benzaldehyde and N4-benzyl-6-(1-methylcyclopropoxy)pyrimidine-4,5-diamine (Synthetic Example 4, intermediate) in a similar procedure to Synthetic Example P2.

(R)-2-Chloro-4-(2-(2-methyl-3-oxopiperazin-1-yl)ethoxy)benzaldehyde was made by N-alkylation using a similar procedure used in the synthesis of (S)-9-benzyl-8-(2-chloro-4-(2-(3-methylpiperazin-1-yl)ethoxy)phenyl)-6-(1-methylcyclopropoxy)-9H-purine (Synthetic Example R3), using (R)-3-methylpiperazin-2-one and 4-(2-bromoethoxy)-2-chlorobenzaldehyde (Synthetic Example 18, intermediate) as starting material. ¹H NMR (300 MHz, DMSO-d₆): 1.23 (3H, d), 2.56-2.66 (1H, m), 2.74-2.87 (1H, m), 2.92-3.04 (2H, m), 3.06-3.17 (3H, m), 4.21-4.29 (2H, m), 7.10 (1H, dd), 7.22 (1H, d), 7.66 (1H, s), 7.83 (1H, d), 10.20 (1H, s). m/z: ES+[M+H]+ 297.

The following examples in Table J were synthesised as stated in the notes at the bottom of Table J.

TABLE J

| | | | | LCMS |
|---|---|---|---|---|
| Ex# | Structure | Name | ¹H NMR | [M + H]+ |
| J1 | 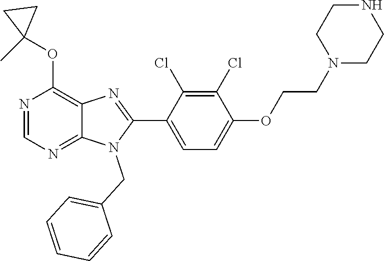 [SMILES: CC1(Oc2ncnc3c2nc(-c2ccc(OCCN4CCNCC4)c(Cl)c2Cl)n3Cc2ccccc2)CC1] | 9-benzyl-8-(2,3- dichloro-4-(2- (piperazin-1- yl)ethoxy) phenyl)-6-(1- methylcyclo- propoxy)-9H- purine | (300 MHz, DMSO-d₆): 0.85 (2H, t), 1.04 (2H, t), 1.67 (3H, s), 1.94-2.30 (1H, m), 2.49- 2.51 (4H, m), 2.65- 2.74 (6H, m), 4.28 (2H, t), 5.28 (2H, s), 6.88-6.92 (2H, m), 7.19- 7.28 (3H, m), 7.31 (1H, d), 7.45 (1H, d), 8.63 (1H, s). | 553 |
| J2 | 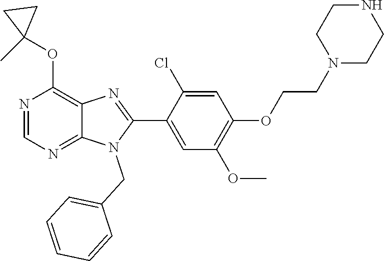 [SMILES: COc1cc(-c2nc3c(OC4(C)CC4)ncnc3n2Cc2ccccc2)c(Cl)cc1OCCN1CCNCC1] | 9-benzyl-8-(2- chloro-5- methoxy-4-(2- (piperazin-1- yl)ethoxy) phenyl)-6-(1- methylcyclo- propoxy)-9H- purine | (300 MHz, DMSO-d₆): 0.86 (2H, t), 1.03 (2H, t), 1.74 (3H, s), 2.35-2.48 (4H, m), 2.64- 2.72 (6H, m), 3.61 (3H, s), 4.17 (2H, t), 5.31 (2H, s), 6.9-6.96 (3H, m), 7.2-7.27 (4H, m), 8.63 (1H, s). One proton not seen. | 549 |
| J3 | 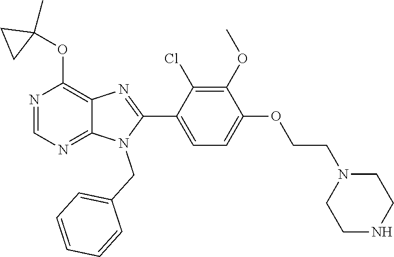 [SMILES: COc1c(OCCN2CCNCC2)ccc(-c2nc3c(OC4(C)CC4)ncnc3n2Cc2ccccc2)c1Cl] | 9-benzyl-8-(2- chloro-3- methoxy-4-(2- (piperazin-1- yl)ethoxy) phenyl)-6-(1- methylcyclo- propoxy)-9H- purine | (300 MHz, DMSO-d₆): 0.65-0.94 (2H, m), 0.94- 1.13 (2H, m), 1.72 (3H, s), 2.21-2.46 (4H, m), 2.58-2.86 (6H, m), 3.81 (3H, s), 4.21 (2H, t), 5.27 (2H, s), 6.71-7 (2H, m), 7-7.47 (5H, m), 8.61 (1H, s). One proton not seen. | 549 |
| J4 | 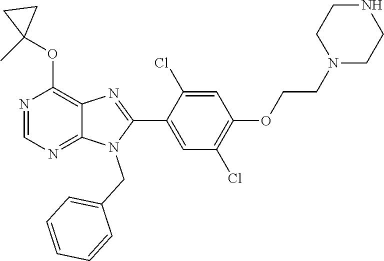 [SMILES: CC1(Oc2ncnc3c2nc(-c2cc(Cl)c(OCCN4CCNCC4)cc2Cl)n3Cc2ccccc2)CC1] | 9-benzyl-8-(2,5- dichloro-4-(2- (piperazin-1- yl)ethoxy) phenyl)-6-(1- methylcyclo- propoxy)-9H- purine | (300 MHz, DMSO-d₆): 0.79 (2H, t), 1.00 (2H, t), 1.71 (3H, s), 2.39-2.45 (4H,m), 2.68 (6H, m), 4.27 (2H, t), 5.29 (2H, s), 6.91 (2H, m), 7.19 (3H, m), 7.48 (1H, s), 7.57 (1H, s), 8.62 (1H, s). One proton not seen. | 553 |
| J5 | 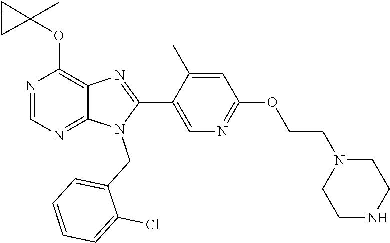 [SMILES: Cc1cc(OCCN2CCNCC2)ncc1-c1nc2c(OC3(C)CC3)ncnc2n1Cc1ccccc1Cl] | 9-(2- chlorobenzyl)-8- (4-methyl-6-(2- (piperazin-1- yl)ethoxy) pyridin- 3-yl)-6-(1- methylcyclo- propoxy)-9H- purine | (400 MHz, DMSO-d₆): 0.86 (2H, t), 1.03 (2H, t), 1.74 (3H, s), 1.99 (3H, s), 2.36-2.40 (4H, m), 2.60-2.71 (6H, m), 4.38 (2H, t), 5.44 (2H, s), 6.73- 6.81 (2H, m), 7.16 (1H, t), 7.27 (1H, td), 7.38 (1H, dd), 8.12 (1H, s), 8.62 (1H, s). 1H not observed. | 534 |
| J6 | 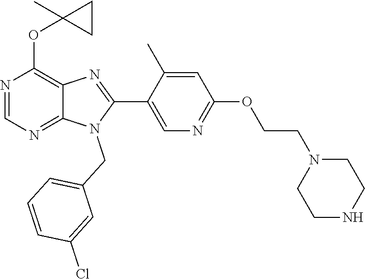 [SMILES: Cc1cc(OCCN2CCNCC2)ncc1-c1nc2c(OC3(C)CC3)ncnc2n1Cc1cccc(Cl)c1] | 9-(3- chlorobenzyl)-8- (4-methyl-6-(2- (piperazin-1- yl)ethoxy) pyridin- 3-yl)-6-(1- methylcyclo- propoxy)-9H- purine | (400 MHz, DMSO-d₆): 0.79-1.02 (4H, m), 1.73 (3H, s), 1.95 (3H, s), 2.31-2.40 (4H, m), 2.57-2.79 (6H, m), 4.30-4.51 (2H, m), 5.33 (2H, s), 6.73-7.05 (3H, m), 7.25-7.27 (2H, m), 8.10 (1H, s), 8.63 (1H, s). 1H not observed. | 534 |
| J7 | 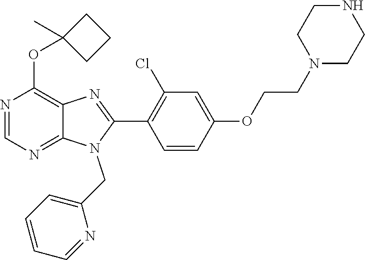 [SMILES: CC1(Oc2ncnc3c2nc(-c2ccc(OCCN4CCNCC4)cc2Cl)n3Cc2ccccn2)CCC1] | 8-(2-chloro-4-(2- (piperazin-1- yl)ethoxy) phenyl)-6-(1- methylcyclo- butoxy)- 9-(pyridin-2- ylmethyl)-9H- purine | (400 MHz, DMSO- d₆): 1.77 (5H, m), 2.28- 2.43 (6H, m), 2.46- 2.49 (2H, m), 2.66- 2.71 (6H, m), 4.14 (2H, t), 5.38 (2H, s), 6.97 (1H, dd), 7.05 (1H, d), 7.21 (2H, m), 7.38 (1H, d), 7.60-7.70 (1H, m), 8.34 (1H, d), 8.45 (1H, s). 1H not observed. | 534 |
| J8 | 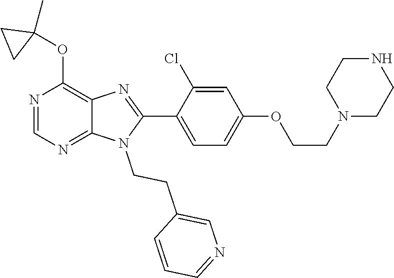 [SMILES: CC1(Oc2ncnc3c2nc(-c2ccc(OCCN4CCNCC4)cc2Cl)n3CCc2cccnc2)CC1] | 8-(2-chloro-4-(2- (piperazin-1- yl)ethoxy) phenyl)-6-(1- methylcyclo- propoxy)-9-(2- (pyridin-3- yl)ethyl)-9H- purine | (300 MHz, CDCl₃): 0.71 (2H, t), 1.03 (2H, t), 1.68 (3H, s), 2.39- 2.52 (4H, m), 2.71 (2H, t), 2.76-2.9 (4H, m), 3.16 (2H, t), 4.02 (2H, t), 4.43 (2H, t), 6.62-6.69 (2H, m), 6.79 (1H, d), 6.89 (1H, d), 6.92-6.99 (1H, m), 7.28-7.36 (1H, m), 8.23 (1H, d), 8.54 (1H, s). One proton not observed. | 534 |
| J9 | 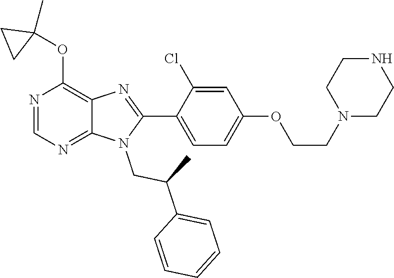 [SMILES: C[C@H](Cn1c(-c2ccc(OCCN3CCNCC3)cc2Cl)nc2c(OC3(C)CC3)ncnc21)c1ccccc1] | (S)-8-(2-chloro- 4-(2-(piperazin- 1- yl)ethoxy) phenyl)-6-(1- methylcyclo- propoxy)-9-(2- phenylpropyl)- 9H-purine | (400 MHz, CDCl₃): 0.83 (2H, t), 1.13-1.26 (5H, m), 1.82 (3H, s), 2.58-2.72 (4H, m), 2.87 (2H, t), 3.02 (4H, t), 3.4-3.49 (1H, m), 4.02-4.12 (1H, m), 4.17 (2H, t), 4.36-4.43 (1H, m), 6.61-6.92 (4H, m), 7.02 (1H, d), 7.11-7.18 (3H, m), 8.69 (1H, s). One proton not observed. | 547 |
| J10 | 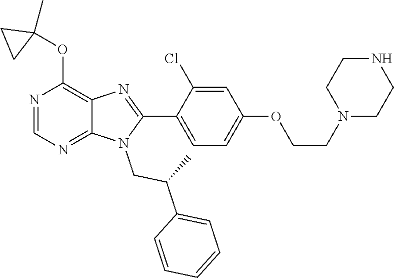 [SMILES: C[C@@H](Cn1c(-c2ccc(OCCN3CCNCC3)cc2Cl)nc2c(OC3(C)CC3)ncnc21)c1ccccc1] | (R)-8-(2-chloro- 4-(2-(piperazin- 1- yl)ethoxy) phenyl)-6-(1- methylcyclo- propoxy)-9-(2- phenylpropyl)- 9H-purine | (300 MHz, DMSO-d₆): 0.83 (2H, t), 0.99 (2H, t), 1.10 (3 H, d), 1.72 (3H, s), 2.39-2.47 (4H, m), 2.61-2.79 (6 H, m), 3.09-3.25 (2 H, m), 4.01-4.39 (4 H, m), 6.79-6.89 (2 H, m), 6.99-7.05 (2H, m), 7.09-7.20 (3 H, m), 7.26-7.29 (1 H, m), 8.62 (1 H, s). | 547 |
| J11 | 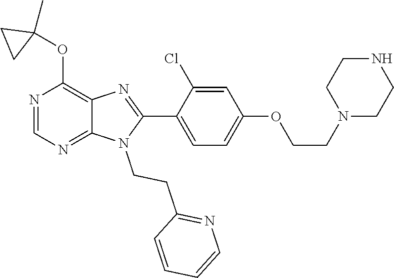 [SMILES: CC1(Oc2ncnc3c2nc(-c2ccc(OCCN4CCNCC4)cc2Cl)n3CCc2ccccn2)CC1] | 8-(2-chloro-4-(2- (piperazin-1- yl)ethoxy) phenyl)-6-(1- methylcyclo- propoxy)-9-(2- (pyridin-2- yl)ethyl)-9H- purine | (400 MHz, DMSO-d₆): 0.84 (2H, t), 1.00 (2H, t), 1.72 (3H, s), 2.38-2.43 (4H, m), 2.67- 2.70 (6H, m), 3.15 (2H, t), 4.19 (2H, t), 4.42 (2H, t), 6.94-7.02 (2H, m), 7.13-7.18 (2H, m), 7.26 (1H, s), 7.55-7.59 (1H, m), 8.31 (1H, d), 8.60 (1H, s). One proton not observed. | 534 |
| J12 | 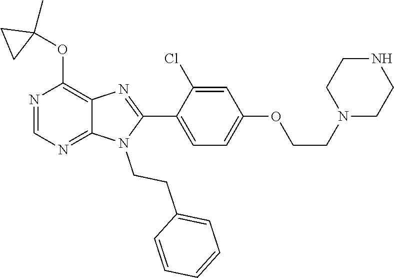 [SMILES: CC1(Oc2ncnc3c2nc(-c2ccc(OCCN4CCNCC4)cc2Cl)n3CCc2ccccc2)CC1] | 8-(2-chloro-4-(2- (piperazin-1- yl)ethoxy) phenyl)-6-(1- methylcyclo- propoxy)-9- phenethyl-9H- purine | (400 MHz, DMSO-d₆): 0.84 (2H, t), 1.01 (2H, t), 1.73 (3H, s), 2.35-2.46 (4H, m), 2.60-2.75 (7H, m), 2.98 (2H, t), 4.14-4.31 (4H, m), 6.79-6.84 (2H, m), 6.93-7.02 (2H, m), 7.13-7.19 (3H, m), 7.27 (1H, s), 8.61 (1H, s). | 533 |
| J13 | 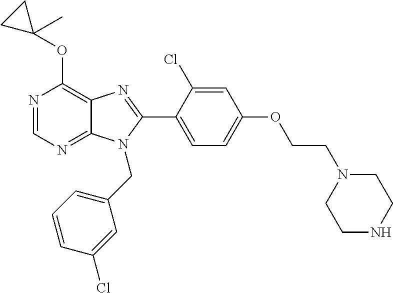 [SMILES: CC1(Oc2ncnc3c2nc(-c2ccc(OCCN4CCNCC4)cc2Cl)n3Cc2cccc(Cl)c2)CC1] | 8-(2-chloro-4-(2- (piperazin-1- yl)ethoxy) phenyl)-9-(3- chlorobenzyl)-6- (1-methyl- cyclopropoxy)- 9H-purine | (400 MHz, DMSO-d₆): 0.85 (2H, t), 1.02 (2H, t), 1.73 (3H, s), 2.39-2.43 (4H, m), 2.64- 2.73 (6H, m), 4.18 (2H, t), 5.29 (2H, s), 6.85 (1H, dt), 6.95 (1H, t), 7.05 (1H, dd), 7.19-7.32 (3H, m), 7.42 (1H, d), 8.63 (1H, s). One proton not observed. | 553 |

[J1] made from a similar procedure (BOC deprotection with TFA) as 9-benzyl-8-(2-chloro-4-(2-(piperazin-1-yl)ethoxy)phenyl)-6-((1,1,1-trifluoro-2-methylpropan-2-yl)oxy)-9H-purine (Synthetic Example 11) using tert-butyl 4-(2-(4-(9-benzyl-6-(1-methylcyclopropoxy)-9H-purin-8-yl)-2,3-dichlorophenoxy)ethyl)piperazine-1-carboxylate as starting material.

Tert-butyl 4-(2-(4-(9-benzyl-6-(1-methylcyclopropoxy)-9H-purin-8-yl)-2,3-dichlorophenoxy)ethyl)piperazine-1-carboxylate was made from a similar procedure as 9-benzyl-8-(2-chloro-4-(2-(4-methylpiperazin-1-yl)ethoxy)phenyl)-6-(1-methylcyclopropoxy)-9H-purine (Synthetic Example 4), using tert-butyl (E)-4-(2-(4-(((4-(benzylamino)-6-(1-methylcyclopropoxy)pyrimidin-5-yl)imino)methyl)-2,3-dichlorophenoxy)ethyl)piperazine-1-carboxylate as starting material. ¹H NMR (300 MHz, DMSO-d₆): 0.73 (2H, t), 0.90 (2H, t), 1.27 (9H, s), 1.61 (3H, s), 2.34-2.38 (4H, m), 2.69 (2H, t), 3.09-3.2 (4H, m), 4.17 (2H, t), 5.16 (2H, s), 6.67-6.89 (2H, m), 7.02-7.16 (3H, m), 7.17 (1H, d), 7.35 (1H, d), 8.51 (1H, s). m/z: ES+[M+H]+ 653.

Tert-butyl (E)-4-(2-(4-(((4-(benzylamino)-6-(1-methylcyclopropoxy)pyrimidin-5-yl)imino)methyl)-2,3-dichlorophenoxy)ethyl)piperazine-1-carboxylate made from a similar procedure as (E)-N-benzyl-5-((2-chloro-4-(2-(4-methylpiperazin-1-yl)ethoxy)benzylidene)amino)-6-(1-methylcyclopropoxy)pyrimidin-4-amine (Synthetic Example 4, intermediate), using tert-butyl 4-(2-(2,3-dichloro-4-formylphenoxy)ethyl)piperazine-1-carboxylate and N4-benzyl-6-(1-methylcyclopropoxy)pyrimidine-4,5-diamine (Synthetic Example 4, intermediate) as starting materials. ¹H NMR (300 MHz, DMSO-d₆): 0.77 (2H, t), 0.94 (2H, t), 1.39 (9H, s), 1.65 (3H, s), 2.50 (4H, d), 2.78-2.87 (2H, m), 3.24-3.32 (4H, m), 4.11 (1H, s), 4.24-4.43 (2H, m), 4.67 (2H, d), 7.21-7.31 (4H, m), 7.71-7.85 (1H, m), 8.02-8.18 (1H, m), 8.3-8.56 (1H, m), 9.35 (1H, s), 12.00 (1H, m). m/z: ES+[M+H]+ 655.

Tert-butyl 4-(2-(2,3-dichloro-4-formylphenoxy)ethyl)piperazine-1-carboxylate made from a similar procedure as tert-butyl 4-(2-(3-chloro-4-formylphenoxy)ethyl)piperazine-1-carboxylate (Synthetic Example 4, intermediate), using 2,3-dichloro-4-hydroxybenzaldehyde and tert-butyl 4-(2-chloroethyl)piperazine-1-carboxylate as starting materials. ¹H NMR (300 MHz, DMSO-d₆): 1.27 (9H, s), 2.34-2.40 (4H, m), 2.68 (2H, t), 3.16-3.21 (4H, m), 4.22 (2H, t), 7.24 (1H, d), 7.72 (1H, d), 10.08 (1H, s). m/z: ES+[M+H]+ 403.

[J2] made from a similar procedure as 9-benzyl-8-(2,3-dichloro-4-(2-(piperazin-1-yl)ethoxy)phenyl)-6-(1-methylcyclopropoxy)-9H-purine (Synthetic Example J1) using tert-butyl 4-(2-(4-(9-benzyl-6-(1-methylcyclopropoxy)-9H-purin-8-yl)-5-chloro-2-methoxyphenoxy)ethyl)piperazine-1-carboxylate as starting material.

The intermediates in the synthesis were made following a similar procedure as Synthetic Example J1:

Tert-butyl 4-(2-(5-chloro-4-formyl-2-methoxyphenoxy)ethyl)piperazine-1-carboxylate made from 2-chloro-4-hydroxy-5-methoxybenzaldehyde and tert-butyl 4-(2-chloroethyl)piperazine-1-carboxylate as starting materials. ¹H NMR (300 MHz, DMSO-d₆): 1.27 (9H, s), 2.25-2.34 (4H, m), 2.61-2.65 (2H, m), 3.17-3.21 (4H, m), 3.70 (3H, s), 4.11 (2H, t), 7.06 (1H, s), 7.19 (1H, s), 10.06 (1H, s). m/z: ES+[M+H]+ 399.

Tert-butyl (E)-4-(2-(4-(((4-(benzylamino)-6-(1-methylcyclopropoxy)pyrimidin-5-yl)imino)methyl)-5-chloro-2-methoxyphenoxy)ethyl)piperazine-1-carboxylate was made from tert-butyl 4-(2-(5-chloro-4-formyl-2-methoxyphenoxy)ethyl)piperazine-1-carboxylate and N4-benzyl-6-(1-methylcyclopropoxy)pyrimidine-4,5-diamine (Synthetic Example 4, intermediate) as starting materials. ¹H NMR (300 MHz, DMSO-d₆): 0.74 (2H, d), 0.90 (2H, d), 1.38 (12H, s), 2.45 (4H, s), 2.80 (2H, d), 3.15 (4H, s), 3.85 (3H, s), 4.09 (2H, s), 4.19 (2H, q), 7.16 (1H, s), 7.17-7.32 (5H, m), 7.83 (1H, s), 9.24 (1H, s), 11.94 (2H, s). m/z: ES+[M+H]+ 651.

Tert-butyl 4-(2-(4-(9-benzyl-6-(1-methylcyclopropoxy)-9H-purin-8-yl)-5-chloro-2-methoxyphenoxy)ethyl)piperazine-1-carboxylate made from tert-butyl (E)-4-(2-(4-(((4-(benzylamino)-6-(1-methylcyclopropoxy)pyrimidin-5-yl)imino)methyl)-5-chloro-2-methoxyphenoxy)ethyl)piperazine-1-carboxylate as starting material. ¹H NMR (300 MHz, DMSO-d₆): 0.85 (2H, t), 0.99 (2H, t), 1.39 (9H, s), 1.72 (3H, s), 2.33-2.49 (10H, m), 3.60 (3H, s), 4.1-4.35 (2H, m), 5.29 (2H, s), 6.73-7 (3H, m), 7.1-7.3 (4H, m), 8.61 (1H, s). m/z: ES+[M+H]+ 649.

[J3] made from a similar procedure as 9-benzyl-8-(2,3-dichloro-4-(2-(piperazin-1-yl)ethoxy)phenyl)-6-(1-methylcyclopropoxy)-9H-purine (Synthetic Example J1) using tert-butyl 4-(2-(4-(9-benzyl-6-(1-methylcyclopropoxy)-9H-purin-8-yl)-3-chloro-2-methoxyphenoxy)ethyl)piperazine-1-carboxylate as starting material.

Tert-butyl 4-(2-(4-(9-benzyl-6-(1-methylcyclopropoxy)-9H-purin-8-yl)-3-chloro-2-methoxyphenoxy)ethyl)piperazine-1-carboxylate made from a similar procedure as (E)-N-benzyl-5-((2-chloro-4-(2-(4-methylpiperazin-1-yl)ethoxy)benzylidene)amino)-6-(1-methylcyclopropoxy)pyrimidin-4-amine (Synthetic Example 4, intermediate) followed by a similar procedure as 9-benzyl-8-(2-chloro-4-(2-(4-methylpiperazin-1-yl)ethoxy)phenyl)-6-(1-methylcyclopropoxy)-9H-purine (Synthetic Example 4), using tert-butyl 4-(2-(3-chloro-4-formyl-2-methoxyphenoxy)ethyl)piperazine-1-carboxylate and N4-benzyl-6-(1-methylcyclopropoxy)pyrimidine-4,5-diamine (Synthetic Example 4, intermediate) as starting materials. ¹H NMR (300 MHz, DMSO-d₆): 0.73 (2H, t), 0.89 (2H, t), 1.27 (9H, s), 1.61 (3H, s), 2.28-2.36 (4H, m), 2.67 (2H, t), 3.14-3.2 (4H, m), 3.69 (3H, s), 4.11 (2H, t), 5.16 (2H, s), 6.75-6.8 (2H, m), 7.04-7.13 (5H, m), 8.50 (1H, s). m/z: ES+[M+H]+ 649.

Tert-butyl 4-(2-(3-chloro-4-formyl-2-methoxyphenoxy)ethyl)piperazine-1-carboxylate made from a similar procedure as tert-butyl 4-(2-(3-chloro-4-formylphenoxy)ethyl)piperazine-1-carboxylate (Synthetic Example 4, intermediate), using 2-chloro-4-hydroxy-3-methoxybenzaldehyde and tert-butyl 4-(2-chloroethyl)piperazine-1-carboxylate as starting materials ¹H NMR (300 MHz, DMSO-d₆): 1.39 (9H, s), 2.46 (4H, t), 2.78 (2H, t), 3.30 (4H, t), 3.82 (3H, s), 4.27 (2H, t), 7.27 (1H, d), 7.66 (1H, d), 10.19 (1H, s). m/z: ES+[M+H]+ 399.

[J4] made from a similar procedure as 9-benzyl-8-(2,3-dichloro-4-(2-(piperazin-1-yl)ethoxy)phenyl)-6-(1-methylcyclopropoxy)-9H-purine (Synthetic Example J1) using tert-butyl 4-(2-(4-(9-benzyl-6-(1-methylcyclopropoxy)-9H-purin-8-yl)-2,5-dichlorophenoxy)ethyl)piperazine-1-carboxylate as starting material.

Tert-butyl 4-(2-(4-(9-benzyl-6-(1-methylcyclopropoxy)-9H-purin-8-yl)-2,5-dichlorophenoxy)ethyl)piperazine-1-carboxylate made from a similar procedure as 9-benzyl-8-(2-chloro-4-(2-(4-methylpiperazin-1-yl)ethoxy)phenyl)-6-(1-methylcyclopropoxy)-9H-purine (Synthetic Example 4), using tert-butyl (E)-4-(2-(4-(((4-(benzylamino)-6-(1-methylcyclopropoxy)pyrimidin-5-yl)imino)methyl)-2,5-dichlorophenoxy)ethyl)piperazine-1-carboxylate as starting material. The product was used in next reaction without further purification.

Tert-butyl (E)-4-(2-(4-(((4-(benzylamino)-6-(1-methylcyclopropoxy)pyrimidin-5-yl)imino)methyl)-2,5-dichlorophenoxy)ethyl)piperazine-1-carboxylate made from a similar procedure as (E)-N-benzyl-5-((2-chloro-4-(2-(4-methylpiperazin-1-yl)ethoxy)benzylidene)amino)-6-(1-methylcyclopropoxy)pyrimidin-4-amine (Synthetic Example 4, intermediate), using tert-butyl 4-(2-(2,5-dichloro-4-formylphenoxy)ethyl)piperazine-1-carboxylate and N4-benzyl-6-(1-methylcyclopropoxy)pyrimidine-4,5-diamine (Synthetic Example 4, intermediate) as starting materials. ¹H NMR (300 MHz, DMSO-d₆): 0.75 (2H, t), 0.91 (2H, t), 1.38 (9H, s), 1.63 (3H, s), 3.25-3.32 (6H, m) 3.13-3.18 (4H, m), 4.29 (2H, t), 4.66 (2H, d), 7.29 (5H, d), 7.38 (1H, s), 8.03 (1H, s), 8.54 (1H, s), 9.29 (1H, s), 11.94 (1H, s). m/z: ES+[M+H]+ 655.

Tert-butyl 4-(2-(2,5-dichloro-4-formylphenoxy)ethyl)piperazine-1-carboxylate made from a similar procedure as tert-butyl 4-(2-(3-chloro-4-formylphenoxy)ethyl)piperazine-1-carboxylate (Synthetic Example 4, intermediate), using 2,5-dichloro-4-hydroxybenzaldehyde and tert-butyl 4-(2-chloroethyl)piperazine-1-carboxylate as starting materials. ¹H NMR (300 MHz, DMSO-d₆): 1.27 (9H, s), 2.31-2.37 (4H, m), 2.67 (2H, t), 3.13-3.19 (4H, m), 4.23 (2H, t), 7.35 (1H, s), 7.73 (1H, s), 10.03 (1H, s). m/z: ES+[M+H]+ 403.

[J5]9-(2-Chlorobenzyl)-8-(4-methyl-6-(2-(piperazin-1-yl)ethoxy)pyridin-3-yl)-6-(1-methylcyclopropoxy)-9H-purine was made by the following multi-step sequence:

Iron (334 mg, 5.97 mmol) was added to ammonium chloride (32 mg, 0.60 mmol) and N-(2-chlorobenzyl)-6-(1-methylcyclopropoxy)-5-nitropyrimidin-4-amine (200 mg, 0.60 mmol, Synthetic Example E1 intermediate) in ethanol: H20 (10:1; 0.40 mL) at 25° C. The resulting mixture was stirred at 60° C. for 1 hour. The reaction mixture was filtered, and the filtrate was concentrated under reduced pressure. The residue was added to a mixture of tert-butyl 4-(2-((5-formyl-4-methylpyridin-2-yl)oxy)ethyl)piperazine-1-carboxylate (251 mg, 0.72 mmol) in MeOH: acetic acid (20:1; 4 mL) and stirred at 25° C. for 16 hours. The solvent was removed under reduced pressure to afford crude tert-butyl (E)-4-(2-((5-(((4-((2-chlorobenzyl)amino)-6-(1-methylcyclopropoxy)pyrimidin-5-yl)imino)methyl)-4-methylpyridin-2-yl)oxy)ethyl)piperazine-1-carboxylate as a brown oil, which was used in the next step directly without further purification.

Tert-butyl 4-(2-((5-(9-(2-chlorobenzyl)-6-(1-methylcyclopropoxy)-9H-purin-8-yl)-4-methylpyridin-2-yl)oxy)ethyl)piperazine-1-carboxylate was obtained using a similar procedure as that described for 9-benzyl-8-(2-chloro-4-(2-(4-methylpiperazin-1-yl)ethoxy)phenyl)-6-(1-methylcyclopropoxy)-9H-purine (Synthetic Example 4), using tert-butyl (E)-4-(2-((5-(((4-((2-chlorobenzyl)amino)-6-(1-methylcyclopropoxy)pyrimidin-5-yl)imino)methyl)-4-methylpyridin-2-yl)oxy)ethyl)piperazine-1-carboxylate (350 mg, 0.55 mmol) to afford tert-butyl 4-(2-((5-(9-(2-chlorobenzyl)-6-(1-methylcyclopropoxy)-9H-purin-8-yl)-4-methylpyridin-2-yl)oxy)ethyl)piperazine-1-carboxylate (100 mg, 29%) as a brown oil. ¹H NMR (400 MHz, DMSO-d₆): 0.87 (2H, t), 1.03 (2H, t), 1.40 (9H, s), 1.75 (3H, s), 1.99 (3H, s), 2.42 (4H, t), 2.70 (2H, t), 3.27-3.32 (4H, m), 4.40 (2H, t), 5.43 (2H, s), 6.74-6.81 (2H, m), 7.13-7.18 (1H, m), 7.24-7.28 (1H, m), 7.36-7.4 (1H, m), 8.12 (1H, s), 8.62 (1H, s). m/z: ES+[M+H]+ 634.

Tert-butyl 4-(2-((5-(9-(2-chlorobenzyl)-6-(1-methylcyclopropoxy)-9H-purin-8-yl)-4-methylpyridin-2-yl)oxy)ethyl)piperazine-1-carboxylate underwent BOC deprotection as described for the synthesis of 8-(4-(2-(2,6-diazaspiro[3.3]heptan-2-yl)ethoxy)-2-chlorophenyl)-9-benzyl-6-(1-methylcyclopropoxy)-9H-purine (Synthetic Example 18) to afford 9-(2-chlorobenzyl)-8-(4-methyl-6-(2-(piperazin-1-yl)ethoxy)pyridin-3-yl)-6-(1-methylcyclopropoxy)-9H-purine (28 mg, 33%) as a yellow solid.

Tert-butyl 4-(2-((5-formyl-4-methylpyridin-2-yl)oxy)ethyl)piperazine-1-carboxylate used as a starting material was obtained as described below:

### Tert-butyl 4-(2-((5-bromo-4-methylpyridin-2-yl)oxy)ethyl)piperazine-1-carboxylate

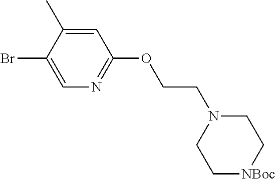 [SMILES: Cc1cc(OCCN2CCN(C(=O)OC(C)(C)C)CC2)ncc1Br]

Sodium hydride (144 mg, 3.60 mmol) was added portionwise to 5-bromo-2-fluoro-4-methylpyridine (570 mg, 3.00 mmol) and tert-butyl 4-(2-hydroxyethyl)piperazine-1-carboxylate (829 mg, 3.60 mmol) in THF (20 mL) at 0° C. over a period of 5 minutes under nitrogen. The resulting solution was stirred at 25° C. for 3 hours. The solvent was removed by distillation under vacuum. The reaction mixture was diluted with water (100 mL) and extracted with EtOAc (3×50 mL). The organic layer was dried over Na₂SO₄, filtered and evaporated to afford crude product. The crude product was purified by flash silica chromatography, elution gradient 0 to 30% EtOAc in petroleum ether. Pure fractions were evaporated to dryness to afford tert-butyl 4-(2-((5-bromo-4-methylpyridin-2-yl)oxy)ethyl)piperazine-1-carboxylate (1000 mg, 83%) as a colourless solid. ¹H NMR (300 MHz, CDCl₃): 1.48 (9H, s), 2.35 (3H, s), 2.56 (4H, t), 2.83 (2H, t), 3.49 (4H, t), 4.44 (2H, t), 6.68 (1H, s), 8.17 (1H, s). m/z: ES+[M+H]+ 400.

### Tert-butyl 4-(2-((5-formyl-4-methylpyridin-2-yl)oxy)ethyl)piperazine-1-carboxylate

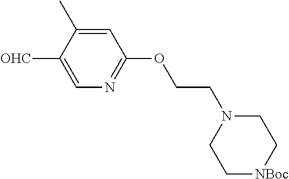 [SMILES: Cc1cc(OCCN2CCN(C(=O)OC(C)(C)C)CC2)ncc1C=O]

A solution of butyl lithium (0.96 mL, 2.40 mmol, 2.5M) in hexane was added portionwise to a stirred solution of tert-butyl 4-(2-((5-bromo-4-methylpyridin-2-yl)oxy)ethyl)piperazine-1-carboxylate (800 mg, 2.00 mmol) in THF (20 mL) at −78° C. over a period of 2 minutes under nitrogen. The resulting solution was stirred at −78° C. for 18 minutes, followed by addition of N,N-dimethylformamide (438 mg, 6.00 mmol). The reaction mixture was diluted with water (20 mL) and extracted with EtOAc (3×15 mL). The organic layer was dried over Na₂SO₄, filtered and evaporated to afford crude product. The crude product was purified by flash silica chromatography, elution gradient 0 to 50% EtOAc in petroleum ether. Fractions containing the desired product were evaporated to dryness to afford crude tert-butyl 4-(2-((5-formyl-4-methylpyridin-2-yl)oxy)ethyl)piperazine-1-carboxylate (300 mg) as a colourless solid which was used directly with no further purification. m/z: ES+[M+H]+ 350.

[J6] was obtained in a similar manner to that described for 9-(2-chlorobenzyl)-8-(4-methyl-6-(2-(piperazin-1-yl)ethoxy)pyridin-3-yl)-6-(1-methylcyclopropoxy)-9H-purine (Synthetic Example J5) except that imine formation step was carried out directly between N4-(3-chlorobenzyl)-6-(1-methylcyclopropoxy)pyrimidine-4,5-diamine (200 mg, 0.66 mmol) and tert-butyl 4-(2-((5-formyl-4-methylpyridin-2-yl)oxy)ethyl)piperazine-1-carboxylate (275 mg, 0.79 mmol, Synthetic Example J5 intermediate) in a similar manner to that described in the synthesis of 9-benzyl-8-(2-chloro-4-(2-(4-methylpiperazin-1-yl)ethoxy)phenyl)-6-(1-methylcyclopropoxy)-9H-purine (Synthetic Example 4).

N4-(3-Chlorobenzyl)-6-(1-methylcyclopropoxy)pyrimidine-4,5-diamine, used as a starting material, was made in a similar manner to N4-benzyl-6-(1-methylcyclopropoxy)pyrimidine-4,5-diamine (Synthetic Example 4, intermediate) from (3-chlorophenyl)methanamine and 4,6-dichloro-5-nitropyrimidine, except that DIEA was used in place of TEA for the first step.

After three steps, this afforded N4-(3-chlorobenzyl)-6-(1-methylcyclopropoxy)pyrimidine-4,5-diamine as a brown oil. ¹H NMR (300 MHz, DMSO-d₆): 0.68 (2H, t), 0.86 (2H, t), 1.60 (3H, s), 4.10 (2H, s), 4.59 (2H, d), 6.75 (1H, t), 7.24-7.38 (4H, m), 7.72 (1H, s). m/z: ES+[M+H]+ 305.

[J7] To a stirred solution of 6-(1-methylcyclobutoxy)-N4-(pyridin-2-ylmethyl)pyrimidine-4,5-diamine (150 mg, 0.53 mmol), 2-chloro-4-(2-(piperazin-1-yl)ethoxy)benzaldehyde (160 mg, 0.60 mmol, Synthetic Example 14 intermediate) in EtOH (3 mL) was added p-toluenesulfonic acid (136 mg, 0.79 mmol) at 60° C. for 2 h. The reaction mixture was diluted with DCM and saturated aq. NaHCO₃ was added. The reaction mixture was extracted with DCM (3×100 mL). The organic layer was dried over Na₂SO₄, filtered and evaporated to afford a white solid. The crude product was purified by preparative HPLC, column: YMC-Actus Triart C18, 30*150 mm, 5 μm; eluting with decreasingly polar mixtures of acetonitrile in water (containing 10 mmol/L NH₄HCO₃ and 0.1% aqueous ammonia). Fractions containing the desired compound were evaporated to dryness to afford 8-(2-chloro-4-(2-(piperazin-1-yl)ethoxy)phenyl)-6-(1-methylcyclobutoxy)-9-(pyridin-2-ylmethyl)-9H-purine (4 mg, 1%) as a white solid.

6-(1-Methylcyclobutoxy)-N4-(pyridin-2-ylmethyl)pyrimidine-4,5-diamine was obtained as follows:

Iron (531 mg, 9.51 mmol) was added to ammonium chloride (51 mg, 0.95 mmol) and 6-(1-methylcyclobutoxy)-5-nitro-N-(pyridin-2-ylmethyl)pyrimidin-4-amine (300 mg, 0.95 mmol) in ethanol/water (10:1; 10 mL) at 25° C. The resulting mixture was stirred at 60° C. for 1 hour. The solvent was removed under reduced pressure and the crude mixture was purified by flash silica chromatography, elution gradient 0 to 60% EtOAc in petroleum ether. Pure fractions were evaporated to dryness to afford 6-(1-methylcyclobutoxy)-N4-(pyridin-2-ylmethyl)pyrimidine-4,5-diamine (170 mg, 63%) as a white solid. ¹H NMR (400 MHz, DMSO-d₆): 1.61 (3H, s), 1.58-1.82 (2H, m), 2.11-2.22 (2H, m), 2.31-2.39 (2H, m), 4.14 (2H, s), 4.65 (2H, d), 6.76-6.79 (1H, m), 7.19-7.31 (2H, m), 7.61 (1H, s), 7.69-7.73 (1H, m), 8.46-8.53 (1H, m). m/z: ES+[M+H]+ 286.

6-(1-Methylcyclobutoxy)-5-nitro-N-(pyridin-2-ylmethyl)pyrimidin-4-amine was obtained as follows:

LHMDS (1M in THF, 9.4 mL, 9.4 mmol) was added dropwise to 6-chloro-5-nitro-N-(pyridin-2-ylmethyl)pyrimidin-4-amine (1 g, 3.76 mmol) and 1-methylcyclobutan-1-ol (0.648 g, 7.53 mmol) in THF (20 mL) at 0° C. under nitrogen. The resulting mixture was stirred at rt for 3 hours. The reaction mixture was concentrated and diluted with EtOAc (100 mL), and washed sequentially with water (100 mL*3) and saturated brine (100 mL). The organic layer was dried over Na₂SO₄, filtered and evaporated to afford crude product. The crude product was purified by flash silica chromatography, elution gradient 0 to 80% EtOAc in petroleum ether. Pure fractions were evaporated to dryness to afford 6-(1-methylcyclobutoxy)-5-nitro-N-(pyridin-2-ylmethyl)pyrimidin-4-amine (300 mg, 25%) as a colourless gum. ¹H NMR (400 MHz, DMSO-d₆): 1.63-1.84 (5H, m), 2.21-2.26 (2H, m), 2.32-2.39 (2H, m), 4.80 (2H, d), 7.23-7.37 (2H, m), 7.74-7.78 (1H, m), 8.22 (1H, s), 8.53 (1H, dd), 9.06 (1H, t). m/z: ES+[M+H]+ 316.

6-Chloro-5-nitro-N-(pyridin-2-ylmethyl)pyrimidin-4-amine was made via a similar procedure to N-benzyl-6-chloro-5-nitropyrimidin-4-amine (Synthetic Example 4 intermediate), starting from 4,6-dichloro-5-nitropyrimidine and pyridin-2-ylmethanamine. m/z: ES+[M+H]+ 266.

[J8] Obtained via a similar procedure to 9-benzyl-8-(2-chloro-4-(2-(4-methylpiperazin-1-yl)ethoxy)phenyl)-6-(1-methylcyclopropoxy)-9H-purine (Synthetic Example 4), using tert-butyl 4-(2-(3-chloro-4-formylphenoxy)ethyl)piperazine-1-carboxylate and 6-(1-methylcyclopropoxy)-N4-(2-(pyridin-3-yl)ethyl)pyrimidine-4,5-diamine, followed by BOC deprotection with t-butyldimethylsilyltrifluoromethanesulfonate (see Synthetic Example 6 for such deprotection).

6-(1-Methylcyclopropoxy)-N4-(2-(pyridin-3-yl)ethyl)pyrimidine-4,5-diamine was made in a similar method as N4-benzyl-6-(1-methylcyclopropoxy)pyrimidine-4,5-diamine (see Synthetic Example 4), starting from 4,6-dichloro-5-nitropyrimidine and 2-(pyridin-3-yl)ethan-1-amine.

¹H NMR (300 MHz, DMSO-d₆): 0.54 (2H, t), 0.72 (2H, t), 1.47 (3H, s), 2.87 (2H, t), 3.52-3.63 (2H, m), 3.88 (2H, s), 6.13 (1H, t), 7.06-7.17 (2H, m), 7.53-7.67 (2H, m), 8.38 (1H, d). m/z: ES+[M+H]+ 286.

[J9] Obtained via a similar procedure as 8-(2-chloro-4-(2-(piperazin-1-yl)ethoxy)phenyl)-6-(1-methylcyclopropoxy)-9-(2-(pyridin-3-yl)ethyl)-9H-purine (Synthetic Example J8), from tert-butyl 4-(2-(3-chloro-4-formylphenoxy)ethyl)piperazine-1-carboxylate and (S)-6-(1-methylcyclopropoxy)-N4-(2-phenylpropyl)pyrimidine-4,5-diamine.

(S)-6-(1-Methylcyclopropoxy)-N4-(2-phenylpropyl)pyrimidine-4,5-diamine was made in a similar method as N4-benzyl-6-(1-methylcyclopropoxy)pyrimidine-4,5-diamine (see Synthetic Example 4), starting from 4,6-dichloro-5-nitropyrimidine and (2S)-2-phenyl-1-propanamine.

¹H NMR (300 MHz, DMSO-d₆): 0.54 (2H, t), 0.71 (2H, t), 1.09 (3H, d), 1.46 (3H, s), 2.88-2.99 (1H, m), 3.25-3.5 (2H, m), 3.92 (2H, s), 6.05 (1H, t), 7.02-7.23 (5H, m), 7.63 (1H, s). m/z: ES+[M+H]+ 299.

[J10] Obtained via a similar procedure as 8-(2-chloro-4-(2-(piperazin-1-yl)ethoxy)phenyl)-6-(1-methylcyclopropoxy)-9-(2-(pyridin-3-yl)ethyl)-9H-purine (Synthetic Example J8), from tert-butyl 4-(2-(3-chloro-4-formylphenoxy)ethyl)piperazine-1-carboxylate and (R)-6-(1-methylcyclopropoxy)-N4-(2-phenylpropyl)pyrimidine-4,5-diamine.

(R)-6-(1-Methylcyclopropoxy)-N4-(2-phenylpropyl)pyrimidine-4,5-diamine was made in a similar method as N4-benzyl-6-(1-methylcyclopropoxy)pyrimidine-4,5-diamine (see Synthetic Example 4), starting from 4,6-dichloro-5-nitropyrimidine and (2R)-2-phenyl-1-propanamine.

¹H NMR (400 MHz, DMSO-d₆): 0.66 (2H, t), 0.84 (2H, t), 1.21 (3H, d), 1.58 (3H, s), 3.00-3.10 (1H, m), 3.39-3.47 (1H, m), 3.50-3.59 (1H, m), 4.02 (2H, s), 6.17 (1H, t), 7.16-7.21 (1H, m), 7.23-7.32 (4H, m), 7.74 (1H, s). m/z: ES+[M+H]+ 299.

[J11] Obtained via a similar procedure as 8-(2-chloro-4-(2-(piperazin-1-yl)ethoxy)phenyl)-6-(1-methylcyclopropoxy)-9-(2-(pyridin-3-yl)ethyl)-9H-purine (Synthetic Example J8), from tert-butyl 4-(2-(3-chloro-4-formylphenoxy)ethyl)piperazine-1-carboxylate and 6-(1-methylcyclopropoxy)-N4-(2-(pyridin-2-yl)ethyl)pyrimidine-4,5-diamine.

6-(1-Methylcyclopropoxy)-N4-(2-(pyridin-2-yl)ethyl)pyrimidine-4,5-diamine was made in a similar method as N4-benzyl-6-(1-methylcyclopropoxy)pyrimidine-4,5-diamine (see Synthetic Example 4), starting from 4,6-dichloro-5-nitropyrimidine and 2-(pyridin-2-yl)ethan-1-amine.

¹H NMR (400 MHz, DMSO-d₆): 0.67 (2H, t), 0.83 (2H, t), 1.59 (3H, s), 2.95-3.10 (2H, m), 3.69-3.74 (2H, m), 3.90-3.99 (2H, m), 6.21-6.28 (1H, m), 7.13-7.34 (2H, m), 7.61-7.82 (2H, m), 8.50 (1H, s). m/z: ES+[M+H]+ 286.

[J12] Obtained via a similar procedure as 8-(2-chloro-4-(2-(piperazin-1-yl)ethoxy)phenyl)-6-(1-methylcyclopropoxy)-9-(2-(pyridin-3-yl)ethyl)-9H-purine (Synthetic Example J8), from 6-(1-methylcyclopropoxy)-N4-phenethylpyrimidine-4,5-diamine and tert-butyl 4-(2-(3-chloro-4-formylphenoxy)ethyl)piperazine-1-carboxylate.

6-(1-Methylcyclopropoxy)-N4-phenethylpyrimidine-4,5-diamine was made in a similar method as N4-Benzyl-6-(1-methylcyclopropoxy)pyrimidine-4,5-diamine (see Synthetic Example 4), starting from 4,6-dichloro-5-nitropyrimidine and 2-phenylethan-1-amine. ¹H NMR (300 MHz, DMSO-d₆): 0.53-0.74 (4H, m), 1.47 (3H, s), 2.72 (2H, t), 3.38-3.49 (2H, m), 3.81-4.00 (2H, m), 6.13 (1H, t), 7.07-7.21 (5H, m), 7.64 (1H, s).

[J13] was obtained via a similar procedure as 8-(2-chloro-4-(2-(piperazin-1-yl)ethoxy)phenyl)-6-(1-methylcyclopropoxy)-9-(2-(pyridin-3-yl)ethyl)-9H-purine (Synthetic Example J8) from a cyclisation of tert-butyl (E)-4-(2-(3-chloro-4-(((4-((3-chlorobenzyl)amino)-6-(1-methylcyclopropoxy)pyrimidin-5-yl)imino)methyl)phenoxy)ethyl)piperazine-1-carboxylate, followed by a BOC deprotection of the resultant tert-butyl 4-(2-(3-chloro-4-(9-(3-chlorobenzyl)-6-(1-methylcyclopropoxy)-9H-purin-8-yl)phenoxy)ethyl)piperazine-1-carboxylate.

Tert-butyl 4-(2-(3-chloro-4-(9-(3-chlorobenzyl)-6-(1-methylcyclopropoxy)-9H-purin-8-yl)phenoxy)ethyl)piperazine-1-carboxylate. ¹H NMR (400 MHz, DMSO-d₆): 0.85 (2H, t), 1.02 (2H, t), 1.40 (9H, s), 1.73 (3H, s), 2.45 (4H, t), 2.74 (2H, t), 3.16-3.19 (4H, m), 4.20 (2H, t), 5.30 (2H, s), 6.85 (1H, dt), 6.9-7 (1H, m), 7.06 (1H, dd), 7.18-7.34 (3H, m), 7.43 (1H, d), 8.63 (1H, s). m/z: ES+[M+H]+ 653.

Tert-butyl (E)-4-(2-(3-chloro-4-(((4-((3-chlorobenzyl)amino)-6-(1-methylcyclopropoxy)pyrimidin-5-yl)imino)methyl)phenoxy)ethyl)piperazine-1-carboxylate was made as follows:

Tert-Butyl 4-(2-(3-chloro-4-formylphenoxy)ethyl)piperazine-1-carboxylate (Synthetic Example 4, intermediate; 1017 mg, 2.76 mmol) was added to N4-(3-chlorobenzyl)-6-(1-methylcyclopropoxy)pyrimidine-4,5-diamine (Synthetic Example J6 intermediate; 700 mg, 2.30 mmol) in MeOH:acetic acid (20:1; 15 mL) at rt. The resulting mixture was stirred at 25° C. for 2 hours. The solvent was removed under reduced pressure to afford yellow oil. The product was used in the next step directly without further purification. ¹H NMR (400 MHz, DMSO-d₆): 0.76 (2H, t), 0.94 (2H, t), 1.40 (9H, s), 1.65 (3H, s), 2.41-2.47 (4H, m), 2.73 (2H, t), 3.32 (4H, t), 4.14-4.27 (2H, m), 4.65 (2H, d), 7.01-7.06 (1H, m), 7.16 (1H, d), 7.24-7.38 (4H, m), 8.06 (1H, s), 8.43 (1H, d), 9.33 (1H, s). One proton not observed. m/z: ES+[M+H]+ 655.

The following examples in Table K were synthesised according to Synthetic Example 23 unless stated otherwise in the notes at the bottom of Table K.

TABLE K

| | | | | LCMS |
|---|---|---|---|---|
| Ex# | Structure | Name | ¹H NMR | [M + H]+ |
| K1 | 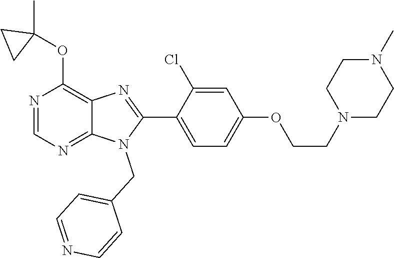 [SMILES: CN1CCN(CCOc2ccc(-c3nc4c(OC5(C)CC5)ncnc4n3Cc3ccncc3)c(Cl)c2)CC1] | 8-(2-chloro-4-(2- (4- methylpiperazin- 1-yl) ethoxy)phenyl)- 6-(1-methyl- cyclopropoxy)- 9-(pyridin-4- ylmethyl)-9H- purine | (400 MHz, DMSO- d₆): 0.79-0.88 (2H, m), 1.02 (2H, d), 1.74 (3H, s), 2.14 (3H, s), 2.15-2.36 (4H, m), 2.36-2.50 (4H, m), 2.69 (2H, t), 4.16 (2H, t), 5.32 (2H, s), 6.85-6.98 (2H, m), 7.03 (1H, dd), 7.24 (1H, d), 7.45 (1H, d), 8.30-8.48 (2H, m), 8.60 (1H, s). | 534 |
| K2 | 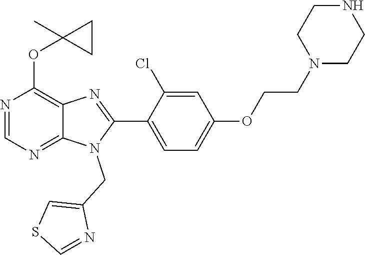 [SMILES: CC1(Oc2ncnc3c2nc(-c2ccc(OCCN4CCNCC4)cc2Cl)n3Cc2cscn2)CC1] | 4-((8-(2-chloro-4- (2-(piperazin-1- yl)ethoxy) phenyl)-6-(1- methylcyclo- propoxy)-9H- purin-9-yl) methyl)thiazole | (400 MHz DMSO- d₆): 0.72-0.87 (2H, m), 1.01 (2H, d), 1.73 (3H, s), 2.40 (4H, s), 2.67 (6H, d), 4.16 (2H, t), 5.41 (2H, s), 6.99-7.04 (1H, m), 7.16-7.33 (2H, m), 7.40 (1H, d), 8.59 (1H, s), 8.91 (1H, d). One proton not observed. | 526 |
| K3 | 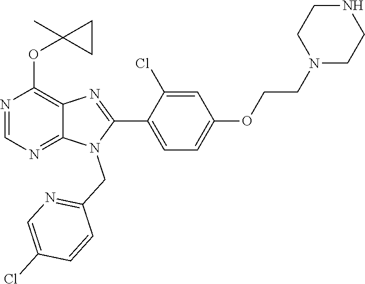 [SMILES: CC1(Oc2ncnc3c2nc(-c2ccc(OCCN4CCNCC4)cc2Cl)n3Cc2ccc(Cl)cn2)CC1] | 8-(2-chloro-4-(2- (piperazin-1- yl)ethoxy) phenyl)-9-((5- chloropyridin-2- yl)methyl)-6-(1- methylcyclo- propoxy)-9H- purine | (400 MHz, DMSO- d₆): 0.85 (2H, t), 1.02 (2H, t), 1.73 (3H, s), 2.34-2.45 (4H, m), 2.62-2.78 (6H, m), 4.15 (2H, t), 5.39 (2H, s), 6.98 (1H, dd), 7.16 (2H, d), 7.37 (1H, d), 7.80 (1H, dd), 8.39 (1H, d), 8.56 (1H, s). One proton not observed. | 554 |
| K4 | 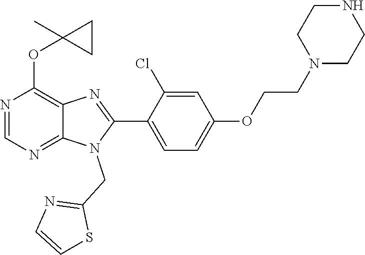 [SMILES: CC1(Oc2ncnc3c2nc(-c2ccc(OCCN4CCNCC4)cc2Cl)n3Cc2nccs2)CC1] | 2-((8-(2-chloro-4- (2-(piperazin-1- yl)ethoxy) phenyl)-6-(1- methylcyclo- propoxy)-9H- purin-9-yl) methyl)thiazole | (400 MHz, DMSO- d₆): 0.85 (2H, t), 1.02 (2H, t), 1.73 (3H, s), 2.38-2.44 (4H, m), 2.66-2.72 (6H, m), 4.17 (2H, t), 5.61 (2H, s), 7.04 (1H, dd), 7.26 (1H, d), 7.43 (1H, d), 7.63 (2H, s), 8.62 (1H, s). One proton not observed. | 526 |
| K5 | 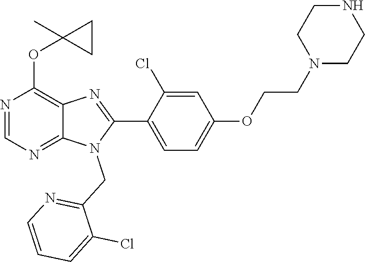 [SMILES: CC1(Oc2ncnc3c2nc(-c2ccc(OCCN4CCNCC4)cc2Cl)n3Cc2ncccc2Cl)CC1] | 8-(2-chloro-4-(2- (piperazin-1- yl)ethoxy) phenyl)-9-((3- chloropyridin-2- yl)methyl)-6-(1- methylcyclo- propoxy)- 9H-purine | (400 MHz, DMSO- d₆): 0.85 (2H, t), 1.03 (2H, t), 1.74 (3H, s), 2.34-2.42 (4H, m), 2.62-2.70 (6H, m), 4.13 (2H, t), 5.54 (2H, s), 6.92 (1H, dd), 7.20 (1H, d), 7.26- 7.32 (2H, m), 7.86 (1H, dd), 8.25 (1H, dd), 8.54 (1H, s). One proton not observed. | 554 |
| K6 | 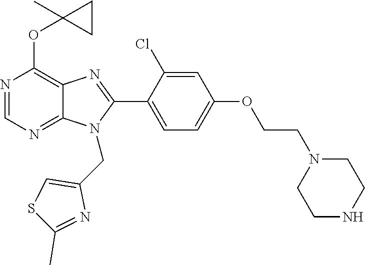 [SMILES: Cc1nc(Cn2c(-c3ccc(OCCN4CCNCC4)cc3Cl)nc3c(OC4(C)CC4)ncnc32)cs1] | 4-((8-(2-chloro-4- (2-(piperazin-1- yl)ethoxy) phenyl)-6-(1- methylcyclo- propoxy)-9H- purin-9- yl)methyl)-2- methylthiazole | (500 MHz, CDCl₃): 0.79-0.84 (2H, m), 1.13- 1.17 (2H, m), 1.79 (3H, s), 2.57 (7H, br s), 2.81 (2H, t), 2.93 (4H, t), 4.14 (2H, t), 5.41 (2H, s), 6.57 (1H, s), 6.85 (1H, dd), 7.03 (1H, d), 7.24-7.36 (1H, m), 8.65 (1H, s). One proton not observed. | 540 |
| K7 | 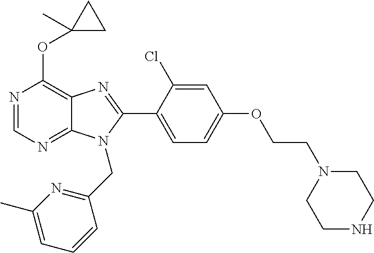 [SMILES: Cc1cccc(Cn2c(-c3ccc(OCCN4CCNCC4)cc3Cl)nc3c(OC4(C)CC4)ncnc32)n1] | 8-(2-chloro-4-(2- (piperazin-1- yl)ethoxy) phenyl)-6-(1- methylcyclo- propoxy)-9-((6- methylpyridin-2- yl)methyl)-9H- purine | (500 MHz, CDCl₃): 0.81-0.85 (2H, m), 1.14-1.19 (2H, m), 1.80 (3H, s), 2.40 (3H, s), 2.55 (4H, s), 2.80 (2H, t), 2.92 (4H, t), 4.12 (2H, t), 5.42 (2H, s), 6.62 (1H, d), 6.80 (1H, dd), 6.95 (1H, d), 7.01 (1H, d), 7.27-7.33 (1H, m), 7.39 (1H, t), 8.62 (1H, s). One proton not observed. | 534 |
| K8 | 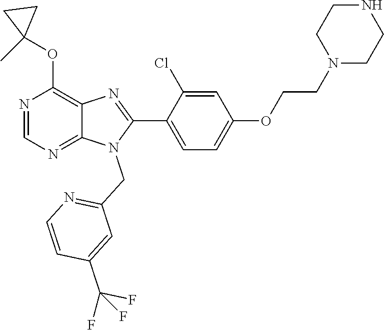 [SMILES: CC1(Oc2ncnc3c2nc(-c2ccc(OCCN4CCNCC4)cc2Cl)n3Cc2cc(C(F)(F)F)ccn2)CC1] | 8-(2-chloro-4-(2- (piperazin-1- yl)ethoxy) phenyl)-6-(1- methylcyclo- propoxy)-9-((4- (trifluoromethyl) pyridin-2- yl)methyl)-9H- purine | (400 MHz, DMSO- d₆): 0.86 (2H, t), 1.04 (2H, t), 1.74 (3H, s), 2.38-2.44 (4H, m), 2.61-2.75 (6H, m), 4.05-4.24 (2H, m), 5.48 (2H, s), 6.91- 7.00 (1H, m), 7.11- 7.22 (1H, m), 7.28- 7.35 (1H, m), 7.48- 7.53 (1H, m), 7.61 (1H, s), 8.53-8.69 (2H, m). One proton not observed. | 588 |
| K9 | 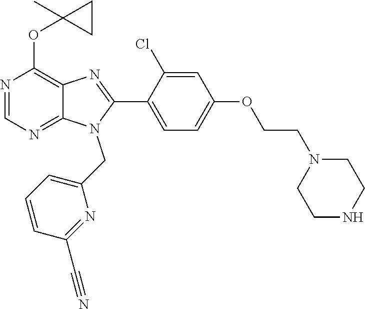 [SMILES: CC1(Oc2ncnc3c2nc(-c2ccc(OCCN4CCNCC4)cc2Cl)n3Cc2cccc(C#N)n2)CC1] | 6-((8-(2-chloro-4- (2-(piperazin-1- yl)ethoxy) phenyl)-6-(1- methylcyclo- propoxy)-9H- purin-9-yl)methyl) picolinonitrile | (500 MHz, CDCl₃): 0.81-0.86 (2H, m), 1.13-1.2 (2H, m), 1.80 (3H, s), 2.56 (4H, br s), 2.81 (2H, t), 2.92 (4H, t), 4.14 (2H, t), 5.48 (2H, s), 6.87 (1H, dd), 7.03 (1H, d), 7.27 (1H, d), 7.35 (1H, d), 7.53 (1H, d), 7.72 (1H, t), 8.60 (1H, s). One proton not observed. | 545 |
| K10 | 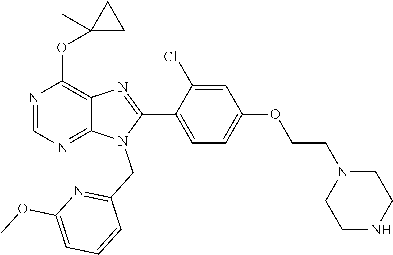 [SMILES: COc1cccc(Cn2c(-c3ccc(OCCN4CCNCC4)cc3Cl)nc3c(OC4(C)CC4)ncnc32)n1] | 8-(2-chloro-4-(2- (piperazin-1- yl)ethoxy) phenyl)-9-((6- methoxypyridin- 2-yl)methyl)-6- (1-methyl- cyclopropoxy)- 9H-purine | (500 MHz, CDCl₃): 0.78-0.86 (2H, m), 1.12-1.2 (2H, m), 1.80 (3H, s), 2.55 (4H, s), 2.80 (2H, t), 2.92 (4H, t), 3.63 (3H, s), 4.12 (2H, t), 5.35 (2H, s), 6.51 (2H, dd), 6.80 (1H, dd), 7.03 (1H, d), 7.32 (1H, d), 7.36-7.41 (1H, m), 8.62 (1H, s). One proton not observed. | 550 |
| K11 | 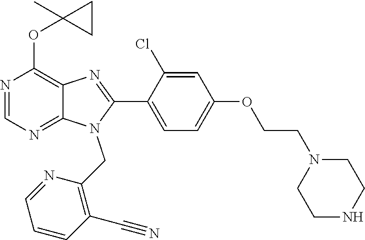 [SMILES: CC1(Oc2ncnc3c2nc(-c2ccc(OCCN4CCNCC4)cc2Cl)n3Cc2ncccc2C#N)CC1] | 2-((8-(2-chloro-4- (2-(piperazin-1- yl)ethoxy) phenyl)-6-(1- methylcyclo- propoxy)-9H- purin-9-yl)methyl) nicotinonitrile | (500 MHz, CDCl₃): 0.8- 0.86 (2H, m), 1.13- 1.19 (2H, m), 1.80 (3H, s), 2.64 (4H, br s), 2.82 (2H, t), 2.96- 3.05 (4H, m), 4.11 (2H, t), 5.69 (2H, s), 6.77 (1H, dd), 7.01 (1H, d), 7.23- 7.26 (1H, m), 7.30 (1H, d), 7.86 (1H, dd), 8.53 (1H, dd), 8.59 (1H, s). One proton not observed. | 545 |
| K12 | 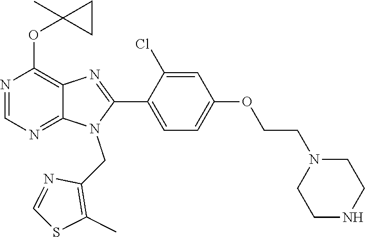 [SMILES: Cc1scnc1Cn1c(-c2ccc(OCCN3CCNCC3)cc2Cl)nc2c(OC3(C)CC3)ncnc21] | 4-((8-(2-chloro-4- (2-(piperazin-1- yl)ethoxy) phenyl)-6-(1- methylcyclo- propoxy)-9H- purin-9- yl)methyl)-5- methylthiazole | (500 MHz, CDCl₃): 0.76-0.85 (2H, m), 1.09- 1.18 (2H, m), 1.78 (3H, s), 2.20 (3H, s), 2.68 (4H, br s), 2.84 (2H, t), 3.05 (4H, br s), 4.13 (2H, t), 5.41 (2H, s), 6.82 (1H, dd), 7.02 (1H, d), 7.24 (1H, d), 8.36 (1H, s), 8.66 (1H, s). One proton not observed. | 540 |

[K1] NaH (65 mg, 2.71 mmol) was added to 8-(2-chloro-4-(2-(4-methylpiperazin-1-yl)ethoxy)phenyl)-6-(1-methylcyclopropoxy)-7H-purine (600 mg, 1.35 mmol) in DMF (10 mL). The resulting mixture was stirred at RT for 10 minutes. Then 4-(chloromethyl)pyridine (259 mg, 2.03 mmol) was added. Finally triethylamine (283 μL, 2.03 mmol) was added. The resulting mixture was stirred at 50° C. for 30 minutes. The reaction mixture was concentrated and diluted with EtOAc (100 mL), and washed sequentially with water (100 mL×3) and saturated brine (100 mL). The organic layer was dried over Na₂SO₄, filtered and evaporated to afford crude product. The crude product was purified by preparative Column: XBridge Prep OBD C18 Column, 30*150 mm, 5 m; using decreasingly polar mixtures of acetonitrile in water (containing 10 mmol/L NH₄HCO₃ and 0.1% aqueous ammonia). Fractions containing the desired compound were evaporated to afford 8-(2-chloro-4-(2-(4-methylpiperazin-1-yl)ethoxy)phenyl)-6-(1-methylcyclopropoxy)-9-(pyridin-4-ylmethyl)-9H-purine (7.9 mg, 11%) as a white solid.

8-(2-Chloro-4-(2-(4-methylpiperazin-1-yl)ethoxy)phenyl)-6-(1-methylcyclopropoxy)-7H-purine was made by a similar method as 4-((8-(2-chloro-4-(2-(4-methylpiperazin-1-yl)ethoxy)phenyl)-6-(1-methylcyclopropoxy)-9H-purin-9-yl)methyl)-2-methylthiazole (see Synthetic Example 7), starting from 6-(1-methylcyclopropoxy)-5-nitropyrimidin-4-amine (see Synthetic Example K₅) and 2-chloro-4-(2-(4-methylpiperazin-1-yl)ethoxy)benzaldehyde (see Synthetic Example 4). m/z: ES+[M+H]+ 443

[K2] Obtained via a similar procedure as 9-benzyl-8-(2-chloro-4-(2-(piperazin-1-yl)ethoxy)phenyl)-6-(1-methylcyclobutoxy)-9H-purine (Synthetic Example 12), using 4-((8-bromo-6-(1-methylcyclopropoxy)-9H-purin-9-yl)methyl)thiazole and tert-butyl 4-(2-(3-chloro-4-(4,4,5,5-tetramethyl-1,3,2-dioxaborolan-2-yl)phenoxy)ethyl)piperazine-1-carboxylate, followed by BOC deprotection using TFA.

4-((8-Bromo-6-(1-methylcyclopropoxy)-9H-purin-9-yl)methyl)thiazole was made as follows:

Cesium carbonate (726 mg, 2.23 mmol) was added dropwise to 4-(chloromethyl)thiazole hydrochloride (316 mg, 1.86 mmol) and 8-bromo-6-(1-methylcyclopropoxy)-9H-purine (500 mg, 1.86 mmol, obtained via free basing of the HBr salt, see Synthetic Example 23) in DMF (10 mL) at 0° C. under nitrogen. The resulting mixture was stirred at rt for 16 hours. The reaction mixture was poured into water (500 mL) and extracted with EtOAc (3×500 mL). The organic layer was dried over Na₂SO₄, filtered and evaporated to afford a brown liquid. The crude product was purified by flash silica chromatography, elution gradient 50 to 100% EtOAc in petroleum ether. Pure fractions were evaporated to dryness to afford 4-((8-bromo-6-(1-methylcyclopropoxy)-9H-purin-9-yl)methyl)thiazole (90 mg, 13%). ¹H NMR (300 MHz, DMSO-d₆): 0.71 (2H, t), 0.86 (2H, t), 1.58 (3H, s), 5.45 (2H, d), 7.49 (1H, d), 8.43 (1H, s), 8.91 (1H, d). m/z: ES+[M+H]+ 366.

[K3] Obtained via a similar procedure as 8-(2-chloro-4-(2-(piperazin-1-yl)ethoxy)phenyl)-9-((6-chloropyridin-2-yl)methyl)-6-(1-methylcyclopropoxy)-9H-purine (Synthetic Example 23), using tert-butyl 4-(2-(3-chloro-4-(6-(1-methylcyclopropoxy)-9H-purin-8-yl)phenoxy)ethyl)piperazine-1-carboxylate and 5-chloro-2-(chloromethyl)pyridine

[K4] Obtained via a similar procedure as 8-(2-chloro-4-(2-(piperazin-1-yl)ethoxy)phenyl)-9-((6-chloropyridin-2-yl)methyl)-6-(1-methylcyclopropoxy)-9H-purine (Synthetic Example 23), using tert-butyl 4-(2-(3-chloro-4-(6-(1-methylcyclopropoxy)-9H-purin-8-yl)phenoxy)ethyl)piperazine-1-carboxylate and 2-(chloromethyl)thiazole

[K5] Obtained via a similar sequence as 2-(2-chloro-4-(2-(piperazin-1-yl)ethoxy)phenyl)-3-((4-chloropyridin-2-yl)methyl)-7-(1-methylcyclopropoxy)-3H-imidazo[4,5-b]pyridine (Synthetic Example 14), starting from N-((3-chloropyridin-2-yl)methyl)-6-(1-methylcyclopropoxy)-5-nitropyrimidin-4-amine.

N-((3-Chloropyridin-2-yl)methyl)-6-(1-methylcyclopropoxy)-5-nitropyrimidin-4-amine used at the start of the sequence was made as follows:

### N-(4-methoxybenzyl)-6-(1-methylcyclopropoxy)-5-nitropyrimidin-4-amine

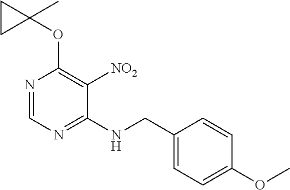 [SMILES: COc1ccc(CNc2ncnc(OC3(C)CC3)c2[N+](=O)[O-])cc1]

LHMDS (75 mL, 75 mmol) was added dropwise to 6-chloro-N-(4-methoxybenzyl)-5-nitropyrimidin-4-amine (10 g, 33.9 mmol; Bioorg. Med. Chem., 2010, 18, 3885-3897) and 1-methylcyclopropan-1-ol (3.67 g, 50.9 mmol) in THF (150 mL) at 0° C. under nitrogen. The resulting mixture was stirred at rt for 3 hours. The reaction mixture was then concentrated under reduced pressure and diluted with EtOAc (100 mL), and washed sequentially with water (100 mL*3) and saturated brine (100 mL). The organic layer was dried over Na₂SO₄, filtered and evaporated to afford crude product. The crude product was purified by flash silica chromatography, elution gradient 0 to 80% EtOAc in petroleum ether. Pure fractions were evaporated to dryness to afford N-(4-methoxybenzyl)-6-(1-methylcyclopropoxy)-5-nitropyrimidin-4-amine (9.0 g, 80%) as a colourless gum. ¹H NMR (400 MHz, DMSO-d₆): 0.70-0.80 (2H, t), 0.91 (2H, t), 1.61 (3H, s), 3.71 (3H, s), 4.61 (2H, d), 6.82-6.92 (2H, m), 7.19-7.31 (2H, m), 8.33 (1H, s), 8.78 (1H, t). m/z: ES+[M+H]+ 331.

### 6-(1-methylcyclopropoxy)-5-nitropyrimidin-4-amine

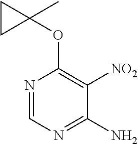

N-(4-methoxybenzyl)-6-(1-methylcyclopropoxy)-5-nitropyrimidin-4-amine (8.5 g, 25.7 mmol) was added into DCM (50 mL) and trifluoroacetic acid (50 mL). The resulting mixture was stirred at 80° C. for 3 hours. The reaction mixture was then cooled to RT and evaporated to afford crude product. The crude product was purified by flash C18-flash chromatography, elution gradient 0 to 100% MeCN in water (containing 0.05% TFA). Pure fractions were evaporated to dryness to afford 6-(1-methylcyclopropoxy)-5-nitropyrimidin-4-amine (4.25 g, 79%) as a yellow solid. ¹H NMR (300 MHz, DMSO-d₆): 0.76 (2H, t), 0.91 (2H, t), 1.62 (3H, s), 8.01 (2H, s), 8.25 (1H, s). m/z: ES+[M+H]+ 211.

### N-((3-chloropyridin-2-yl)methyl)-6-(1-methylcyclopropoxy)-5-nitropyrimidin-4-amine

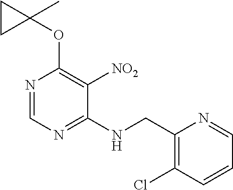 [SMILES: CC1(Oc2ncnc(NCc3ncccc3Cl)c2[N+](=O)[O-])CC1]

6-(1-methylcyclopropoxy)-5-nitropyrimidin-4-amine (300 mg, 1.43 mmol) and 3-chloro-2-(chloromethyl)pyridine (347 mg, 2.14 mmol) was added into DMF (6 mL) and Cs₂CO₃ (930 mg, 2.85 mmol). The resulting mixture was stirred at 100° C. for 1 hour. The reaction mixture was poured into aq. sat. NaHCO₃ (40 mL) and extracted with ethyl acetate (20 mL×3). The combined organic layers were washed with brine (20 mL×2), dried over Na₂SO₄, filtered and concentrated under reduced pressure to give the crude product. The crude product was purified by flash C18-flash chromatography, elution gradient 0 to 100% MeCN in water. Pure fractions were evaporated to dryness to afford N-((3-chloropyridin-2-yl)methyl)-6-(1-methylcyclopropoxy)-5-nitropyrimidin-4-amine (255 mg, 53%) as a pale yellow solid. m/z: ES+[M+H]+ 336.

The following examples in Table L were synthesised according to Synthetic Example L1 unless stated otherwise in the notes at the bottom of Table L.

TABLE L

| | | | | LCMS |
|---|---|---|---|---|
| Ex# | Structure | Name | ¹H NMR | [M + H]+ |
| L1 | 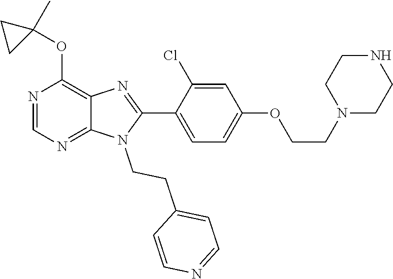 [SMILES: CC1(Oc2ncnc3c2nc(-c2ccc(OCCN4CCNCC4)cc2Cl)n3CCc2ccncc2)CC1] | 8-(2-chloro-4-(2- (piperazin-1- yl)ethoxy)phenyl)- 6-(1-methyl- cyclopropoxy)- 9-(2-(pyridin-4- yl)ethyl)-9H- purine | (400 MHz, CD₃OD): 0.87 (2H, t), 1.10 (2H, t), 1.80 (3H, s), 2.61-2.68 (4H, m), 2.87 (2H, t), 2.93 (4H, t), 3.14 (2H, t), 4.25 (2H, t), 4.48 (2H, t), 6.9-6.94 (2H, m), 6.98 (2H, d), 7.22 (1H, t), 8.27- 8.3 (2H, m), 8.60 (1H, s). 1 H not observed | 534 |
| L2 | 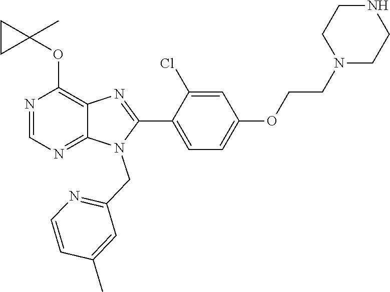 [SMILES: Cc1ccnc(Cn2c(-c3ccc(OCCN4CCNCC4)cc3Cl)nc3c(OC4(C)CC4)ncnc32)c1] | 8-(2-chloro-4-(2- (piperazin-1- yl)ethoxy)phenyl)- 6-(1-methyl- cyclopropoxy)- 9-((4- methylpyridin-2- yl)methyl)-9H- purine | (500 MHz, CDCl₃): 0.8-0.86 (2H, m), 1.13-1.2 (2H, m), 1.81 (3H, s), 2.21 (3H, s), 2.55 (4H, br s), 2.79 (2H, t), 2.86- 2.97 (4H, m), 4.11 (2H, t), 5.43 (2H, s), 6.65 (1H, s), 6.77 (1H, dd), 6.91 (1H, d), 7.01 (1H, d), 7.24 (1H, d), 8.24 (1H, d), 8.64 (1H, s). 1 H not observed | 534 |
| L3 | 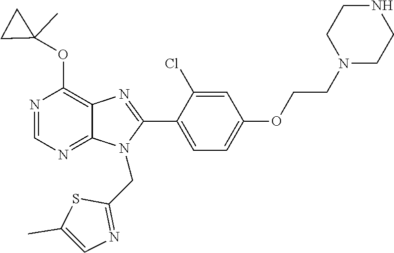 [SMILES: Cc1cnc(Cn2c(-c3ccc(OCCN4CCNCC4)cc3Cl)nc3c(OC4(C)CC4)ncnc32)s1] | 2-((8-(2-chloro-4- (2-(piperazin-1- yl)ethoxy)phenyl)- 6-(1-methyl- cyclopropoxy)- 9H-purin-9- yl)methyl)-5- methylthiazole | (500 MHz, CDCl₃): 0.79-0.85 (2H, m), 1.15 (2H, t), 1.79 (3H, s), 2.35 (3H, s), 2.56 (4H, s), 2.81 (2H, t), 2.93 (4H, t), 4.14 (2H, t), 5.55 (2H, s), 6.87 (1H, dd), 7.05 (1H, d), 7.24 (1H, s), 7.35 (1H, d), 8.67 (1H, s). 1 H not observed | 540 |
| L4 | 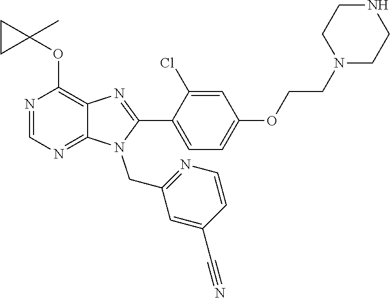 [SMILES: CC1(Oc2ncnc3c2nc(-c2ccc(OCCN4CCNCC4)cc2Cl)n3Cc2cc(C#N)ccn2)CC1] | 2-((8-(2-chloro-4- (2-(piperazin-1- yl)ethoxy)phenyl)- 6-(1-methyl- cyclopropoxy)- 9H-purin-9- yl)methyl) isonicotinonitrile | (500 MHz, CDCl₃): 0.8-0.87 (2H, m), 1.15-1.19 (2H, m), 1.81 (3H, s), 2.49- 2.62 (4H, m), 2.81 (2H, t), 2.9-2.96 (4H, m), 4.12 (2H, t), 5.50 (2H, s), 6.82 (1H, dd), 7.02 (1H, d), 7.12-7.16 (1H, m), 7.27 (1H, d), 7.35 (1H, dd), 8.59 (1H, dd), 8.62 (1H, s). 1H not observed. | 545 |
| L5 | 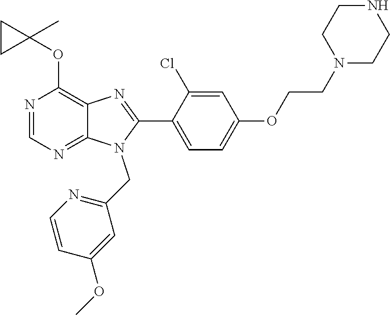 [SMILES: COc1ccnc(Cn2c(-c3ccc(OCCN4CCNCC4)cc3Cl)nc3c(OC4(C)CC4)ncnc32)c1] | 8-(2-chloro-4-(2- (piperazin-1- yl)ethoxy)phenyl)- 9-((4- methoxypyridin-2- yl)methyl)-6-(1- methylcyclopro- poxy)-9H-purine | (500 MHz, DMSO- d₆): 0.78-0.86 (2H, m), 1.14-1.19 (2H, m), 1.80 (3H, s), 2.51- 2.58 (4H, m), 2.79 (2H, t), 2.9-2.93 (4H, m), 3.72 (3H, s), 4.11 (2H, t), 5.41 (2H, s), 6.36 (1H, d), 6.62 (1H, dd), 6.79 (1H, dd), 7.01 (1H, d), 7.26- 7.28 (1H, m), 8.23 (1H, d), 8.64 (1H, s). 1H not observed. | 550 |
| L6 | 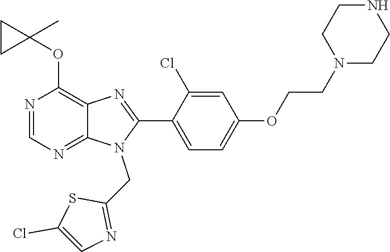 [SMILES: CC1(Oc2ncnc3c2nc(-c2ccc(OCCN4CCNCC4)cc2Cl)n3Cc2ncc(Cl)s2)CC1] | 5-chloro-2-((8-(2- chloro-4-(2- (piperazin-1- yl)ethoxy)phenyl)- 6-(1-methyl- cyclopropoxy)- 9H-purin-9- yl)methyl)thiazole | (500 MHz, DMSO- d₆): 0.81-0.87 (2H, m), 0.99-1.04 (2H, m), 1.72 (3H, s), 2.37- 2.46 (4H, m), 2.68 (2H, t), 2.7-2.75 (4H, m), 4.18 (2H, t), 5.54 (2H, s), 7.06 (1H, dd), 7.25 (1H, d), 7.44 (1H, d), 7.64 (1H, s), 8.61 (1H, s). 1H not observed. | 560 |
| L7 | 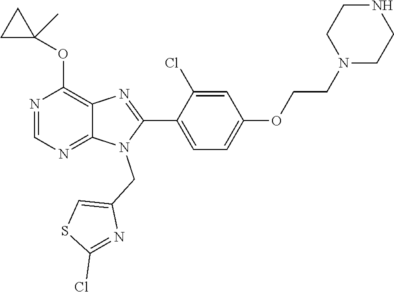 [SMILES: CC1(Oc2ncnc3c2nc(-c2ccc(OCCN4CCNCC4)cc2Cl)n3Cc2csc(Cl)n2)CC1] | 2-chloro-4-((8-(2- chloro-4-(2- (piperazin-1- yl)ethoxy)phenyl)- 6-(1-methyl- cyclopropoxy)- 9H-purin-9- yl)methyl)thiazole | (500 MHz, CDCl₃): 0.8-0.85 (2H, m), 1.13-1.18 (2H, m), 1.79 (3H, s), 2.59 (4H, s), 2.82 (2H, t), 2.95 (4H, t), 4.15 (2H, t), 5.38 (2H, s), 6.75 (1H, s), 6.88 (1H, dd), 7.04 (1H, d), 7.35 (1H, d), 8.64 (1H, s). 1H not observed. | 560 |
| L8 | 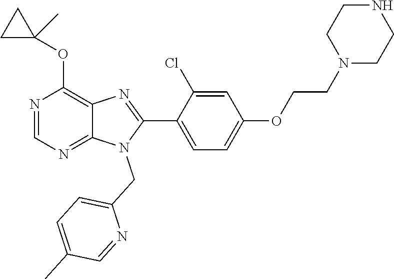 [SMILES: Cc1ccc(Cn2c(-c3ccc(OCCN4CCNCC4)cc3Cl)nc3c(OC4(C)CC4)ncnc32)nc1] | 8-(2-chloro-4-(2- (piperazin-1- yl)ethoxy)phenyl)- 6-(1-methyl- cyclopropoxy)- 9-((5- methylpyridin-2- yl)methyl)-9H- purine | (500 MHz, CDCl₃): 0.79-0.85 (2H, m), 1.14-1.18 (2H, m), 1.80 (3H, s), 2.24 (3H, s), 2.55 (4H, s), 2.80 (2H, t), 2.93 (4H, t), 4.11 (2H, t), 5.42 (2H, s), 6.75 (1H, d), 6.78 (1H, dd), 7.01 (1H, d), 7.25 (1H, s), 7.30 (1H, d), 8.23 (1H, s), 8.63 (1H, d). 1H not observed. | 534 |
| L9 | 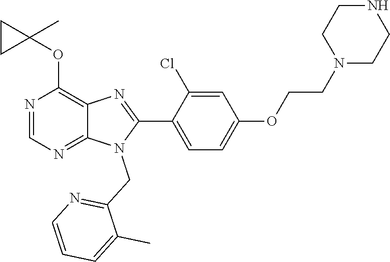 [SMILES: Cc1cccnc1Cn1c(-c2ccc(OCCN3CCNCC3)cc2Cl)nc2c(OC3(C)CC3)ncnc21] | 8-(2-chloro-4-(2- (piperazin-1- yl)ethoxy)phenyl)- 6-(1-methyl- cyclopropoxy)- 9-((3- methylpyridin-2- yl)methyl)-9H- purine | (500 MHz, DMSO- d₆): 0.87-0.92 (2H, m), 1.06-1.11 (2H, m), 1.78 (3H, s), 2.38- 2.44 (4H, m), 2.60 (3H, s), 2.66 (2H, t), 2.68-2.73 (4H, m), 4.13 (2H, t), 5.84 (2H, s), 6.98 (1H, dd), 7.07 (1H, d), 7.22 (1H, dd), 7.67 (1H, d), 7.94 (1H, d), 8.22 (1H, d), 8.87 (1H, s). 1H not observed. | 534 |
| L10 | 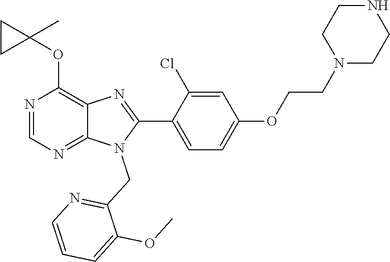 [SMILES: COc1cccnc1Cn1c(-c2ccc(OCCN3CCNCC3)cc2Cl)nc2c(OC3(C)CC3)ncnc21] | 8-(2-chloro-4-(2- (piperazin-1- yl)ethoxy)phenyl)- 9-((3- methoxypyridin-2- yl)methyl)-6-(1- methylcyclopro- poxy)-9H-purine | (500 MHz, CDCl₃): 0.78-0.85 (2H, m), 1.13-1.19 (2H, m), 1.80 (3H, s), 2.56 (4H, s), 2.79 (2H, t), 2.9-2.97 (4H, m), 3.72 (3H, s), 4.09 (2H, t), 5.50 (2H, s), 6.72 (1H, dd), 6.97 (1H, d), 7.00 (1H, dd), 7.06 (1H, dd), 7.28 (1H, d), 7.90 (1H, dd), 8.60 (1H, s). 1H not observed. | 550 |

[L1]8-(2-Chloro-4-(2-(piperazin-1-y1)ethoxy)pheny1)-6-(1-methylcyclopropoxy)-9-(2-(pyridin-4-y1)ethy1)-9H-purine was made as follows:

Diisopropyl azodicarboxylate (DIAD) (344 mg, 1.70 mmol) was added dropwise to tert-butyl 4-(2-(3-Chloro-4-(6-(1-methylcyclopropoxy)-9H-purin-8-y1)phenoxy)ethyl)piperazine-1-carboxylate (150 mg, 0.28 mmol), 2-(pyridin-4-y1)ethan-1-ol (52.4 mg, 0.43 mmol) and triphenylphosphine (223 mg, 0.85 mmol) in THF (3 mL) at 0° C. The resulting mixture was stirred at 0° C. for 1 hour. The reaction mixture was diluted with EtOAc (15 mL), and washed sequentially with water (15 mL X 3). The organic layer was dried over Na₂SO₄, filtered and evaporated to afford crude product. The crude product was purified by flash silica chromatography, elution gradient 0to 10% MeOH in DCM. Pure fractions were evaporated to dryness to afford tert-butyl 4-(2-(3-chloro-4-(6-(1-methylcyclopropoxy)-9-(2-(pyridin-4-yl)ethyl)-9H-purin-8- yl)phenoxy)ethyl)piperazine-1-carboxylate (90 mg, 50 %) as a yellow oil. TFA (0.5 mL) was then added to tert-butyl 4-(2-(3-chloro-4-(6-(1-methylcyclopropoxy)-9-(2-(pyridin-4-yl)ethyl)-9H-purin-8-yl)phenoxy)ethyl)piperazine-1-carboxylate (70 mg, 0.11 mmol) in DCM (2 mL) at 25° C. The resulting mixture was stirred at 25° C. for 1 hour. The solvent was removed under reduced pressure. The crude product was purified by preparative HPLC (XBridge Prep C18 OBD column, 30*150 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness to afford 8-(2-chloro-4-(2-(piperazin-1-yl)ethoxy)phenyl)-6-(1-methylcyclopropoxy)-9-(2-(pyridin-4-yl)ethyl)-9H-purine (21 mg, 36%) as a white solid.

[L2]Obtained from a similar procedure as 8-(2-chloro-4-(2-(piperazin-1-yl)ethoxy)phenyl)-6-(1-methylcyclopropoxy)-9-(2-(pyridin-4-yl)ethyl)-9H-purine (Synthetic Example L1), using tert-butyl 4-(2-(3-chloro-4-(6-(1-methylcyclopropoxy)-9H-purin-8-yl)phenoxy)ethyl)piperazine-1-carboxylate and (4-methylpyridin-2-yl)methanol, except that the Mitsunobu reaction carried out at 60° C.

[L4]was made from a similar procedure as 8-(2-chloro-4-(2-(piperazin-1-yl)ethoxy)phenyl)-6-(1-methylcyclopropoxy)-9-(2-(pyridin-4-yl)ethyl)-9H-purine (Synthetic Example L1), using tert-butyl 4-(2-(3-chloro-4-(6-(1-methylcyclopropoxy)-9H-purin-8-yl)phenoxy)ethyl)piperazine-1-carboxylate (157 mg, 0.30 mmol) and 2-(hydroxymethyl)isonicotinonitrile (80 mg, 0.59 mmol), except that the reagents were combined at ambient temperature and the reaction was stirred then at 60° C. for 2 hours. The crude product was purified by ion exchange chromatography, using an SCX column, and then preparative HPLC purification to afford the BOC protected product. BOC deprotection was carried out using HCl (1N in EtOAc) in EtOAc, followed by further addition of HCl (6N in IPA) and DCM, to afford 2-((8-(2-chloro-4-(2-(piperazin-1-yl)ethoxy)phenyl)-6-(1-methylcyclopropoxy)- 9H-purin-9-yl)methyl)isonicotinonitrile (46 mg, 58%) as a solid.

[L5]was made from a similar procedure to 2-((8-(2-chloro-4-(2-(piperazin-1-yl)ethoxy)phenyl)-6-(1-methylcyclopropoxy)-9H-purin-9- yl)methyl)isonicotinonitrile (Synthetic Example L4) using tert-butyl 4-(2-(3-chloro-4-(6-(1-methylcyclopropoxy)-9H-purin-8-yl)phenoxy)ethyl)piperazine-1-carboxylate (145 mg, 0.27 mmol) and (4-methoxypyridin-2-yl)methanol (76 mg, 0.55 mmol), except that BOC deprotection was carried out in DCM using 6N HCl in iPrOH.

[L6]was made from a similar procedure to 2-((8-(2-chloro-4-(2-(piperazin-1-yl)ethoxy)phenyl)-6-(1-methylcyclopropoxy)-9H-purin-9- yl)methyl)isonicotinonitrile (Synthetic Example L4) using tert-butyl 4-(2-(3-chloro-4-(6-(1-methylcyclopropoxy)-9H-purin-8-yl)phenoxy)ethyl)piperazine-1-carboxylate (151 mg, 0.29 mmol) and (5-chlorothiazol-2-yl)methanol (85 mg, 0.57 mmol), except that BOC deprotection was carried out in DCM using 6N HCl in iPrOH.

[L9]was made from a similar procedure to 2-((8-(2-chloro-4-(2-(piperazin-1-yl)ethoxy)phenyl)-6-(1-methylcyclopropoxy)-9H-purin-9- yl)methyl)isonicotinonitrile (Synthetic Example L4) using tert-butyl 4-(2-(3-chloro-4-(6-(1-methylcyclopropoxy)-9H-purin-8-yl)phenoxy)ethyl)piperazine-1-carboxylate (148 mg, 0.28 mmol) and (3-methylpyridin-2-yl)methanol (69 mg, 0.56 mmol), except that BOC deprotection was carried out in DCM using 6N HCl in iPrOH.

[L10]Obtained from a similar procedure as 8-(2-chloro-4-(2-(piperazin-1-yl)ethoxy)phenyl)-6-(1-methylcyclopropoxy)-9-(2-(pyridin-4-yl)ethyl)-9H- purine [Synthetic Example L1], using tert-butyl 4-(2-(3-chloro-4-(6-(1-methylcyclopropoxy)-9H-purin-8-yl)phenoxy)ethyl)piperazine-1-carboxylate and (3-methoxypyridin-2-yl)methanol, except that the Mitsunobu reaction was carried out at 60 OC

The following examples in Table M were synthesised according the notes at the bottom of Table M.

TABLE M

| | | | | LCMS |
|---|---|---|---|---|
| Ex# | Structure | Name | ¹H NMR | [M + H]⁺ |
| M1 | 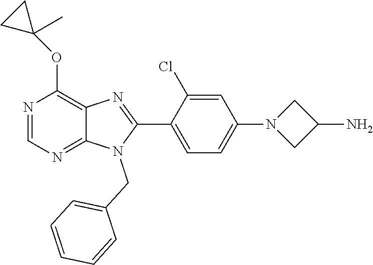 [SMILES: CC1(Oc2ncnc3c2nc(-c2ccc(N4CC(N)C4)cc2Cl)n3Cc2ccccc2)CC1] | 1-(4-(9-benzyl- 6-(1- methylcyclopro- poxy)-9H-purin- 8-yl)-3- chlorophenyl)aze- tidin-3-amine | (400 MHz, DMSO-d₆): 0.80-0.87 (2H, m), 0.98- 1.05 (2H, m), 1.72 (3H, s), 2.23 (2H, s), 3.49 (2H, dd), 3.82 (1H, p), 4.12 (2H, t), 5.28 (2H, s), 6.42 (1H, dd), 6.57 (1H, d), 6.90 (2H, dd), 7.16-7.27 (4H, m), 8.58 (1H, s). | 461 |
| M2 | 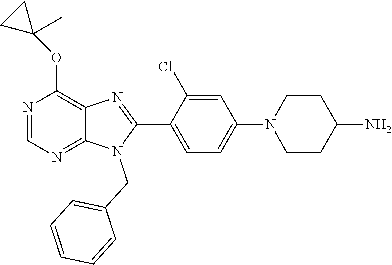 [SMILES: CC1(Oc2ncnc3c2nc(-c2ccc(N4CCC(N)CC4)cc2Cl)n3Cc2ccccc2)CC1] | 1-(4-(9-benzyl- 6-(1-methylcyclopro- poxy)-9H-purin- 8-yl)-3- chlorophenyl)pi- peridin-4-amine | (400 MHz, DMSO-d₆): 0.79-0.88 (2H, m), 0.98- 1.06 (2H, m), 1.23- 1.37 (2H, m), 1.73 (3H, s), 1.75-1.85 (2H, m), 2.81-2.93 (3H, m), 3.80 (2H, dd), 5.30 (2H, s), 6.87-7.00 (3H, m), 7.09 (1H, d), 7.17-7.29 (4H, m), 8.59 (1H, s). Two protons not observed. | 489 |
| M3 | 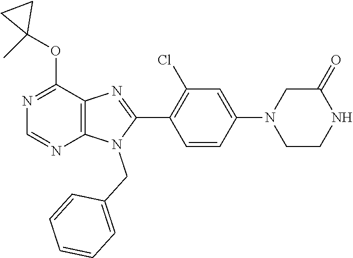 [SMILES: CC1(Oc2ncnc3c2nc(-c2ccc(N4CCNC(=O)C4)cc2Cl)n3Cc2ccccc2)CC1] | 4-(4-(9-benzyl- 6-(1- methylcyclopro- poxy)-9H-purin- 8-yl)-3- chlorophenyl)pi- perazin-2-one | (500 MHz, CDCl₃): 0.8- 0.85 (m, 2H), 1.13- 1.18 (m, 2H), 1.80 (s, 3H), 3.57 (s, 4H), 3.98 (s, 2H), 5.35 (s, 2H), 6.70 (dd, 1H), 6.92 (d, 1H), 6.93-6.96 (m, 2H), 7.14-7.21 (m, 4h), 8.65 (s, 1H). One proton not observed. | 489 |
| M4 | 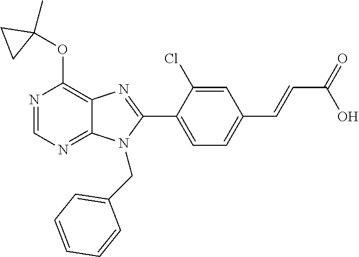 [SMILES: CC1(Oc2ncnc3c2nc(-c2ccc(/C=C/C(=O)O)cc2Cl)n3Cc2ccccc2)CC1] | (E)-3-(4-(9- benzyl-6-(1- methylcyclopro- poxy)-9H-purin- 8-yl)-3- chlorophenyl)a- crylic acid | (300 MHz, DMSO-d₆): 0.79-0.90 (2H, m), 1.02 (2H, d), 1.74 (3H, s), 5.33 (2H, s), 6.75 (1H, d), 6.90 (2H, dd), 7.12-7.26 (3H, m), 7.49- 7.61 (2H, m), 7.76 (1H, dd), 7.99 (1H, d), 8.65 (1H, s). One proton not observed. | 461 |
| M5 | 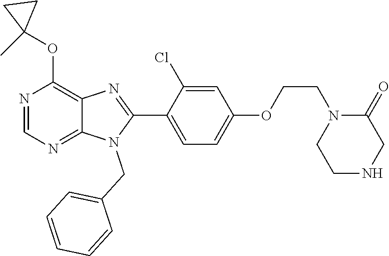 [SMILES: CC1(Oc2ncnc3c2nc(-c2ccc(OCCN4CCNCC4=O)cc2Cl)n3Cc2ccccc2)CC1] | 1-(2-(4-(9- benzyl-6-(1- methylcyclopro- poxy)-9H-purin- 8-yl)-3- chlorophenoxy)eth- yl)piperazin-2- one | (300 MHz, DMSO-d₆): 0.80-0.88 (2H, m), 0.99- 1.05 (2H, m), 1.73 (3H, s), 2.85 (2H, t), 3.22 (2H, s), 3.39 (2H, t), 3.65 (2H, t), 4.23 (2H, t), 5.29 (2H, s), 6.90 (2H, dd), 7.05 (1H, dd), 7.16-7.23 (3H, m), 7.27 (1H, d), 7.42 (1H, d), 8.62 (1H, s). One proton not observed. | 533 |

[M1] was synthesised from tert-butyl (1-(4-(9-benzyl-6-(1-methylcyclopropoxy)-9H-purin-8-yl)-3-chlorophenyl)azetidin-3-yl)carbamate via a BOC deprotection using a similar procedure as used in the synthesis of 2-(2-chloro-4-(2-(piperazin-1-yl)ethoxy)phenyl)-1-((4-chloropyridin-2-yl)methyl)-4-(1-methylcyclopropoxy)-1H-benzo[d]imidazole (Synthetic Example 2).

Tert-butyl (1-(4-(9-benzyl-6-(1-methylcyclopropoxy)-9H-purin-8-yl)-3-chlorophenyl)azetidin-3-yl)carbamate was made following a palladium catalysed cross coupling reaction, using 9-benzyl-8-(4-bromo-2-chlorophenyl)-6-(1-methylcyclopropoxy)-9H-purine and tert-butyl azetidin-3-ylcarbamate as starting materials, as follows:

9-Benzyl-8-(4-bromo-2-chlorophenyl)-6-(1-methylcyclopropoxy)-9H-purine (300 mg, 0.64 mmol), tert-butyl azetidin-3-ylcarbamate (165 mg, 0.96 mmol), Cs₂CO₃ (624 mg, 1.92 mmol) and rac-BINAP Pd G3 (127 mg, 0.13 mmol) in 1,4-dioxane (6 mL) were stirred under an atmosphere of nitrogen at 100° C. for 12 hours. The reaction mixture was concentrated and diluted with water (25 mL) and extracted with EtOAc (3×25 mL). The organic layer was dried over Na₂SO₄, filtered and evaporated to afford crude product. The crude product was purified by flash silica chromatography, elution gradient 0 to 40% EtOAc in pentane. Pure fractions were evaporated to dryness to afford tert-butyl (1-(4-(9-benzyl-6-(1-methylcyclopropoxy)-9H-purin-8-yl)-3-chlorophenyl)azetidin-3-yl)carbamate (0.220 g, 61%) as a yellow solid. ¹H NMR (300 MHz, DMSO-d₆): 0.79-0.88 (2H, m), 1.01 (2H, d), 1.40 (9H, s), 1.72 (3H, s), 3.71 (2H, dd), 4.17 (2H, t), 4.43 (1H, t), 5.28 (2H, s), 6.43 (1H, dd), 6.60 (1H, d), 6.87-6.93 (2H, m), 7.17-7.24 (4H, m), 7.45-7.70 (1H, m), 8.58 (1H, s). m/z: ES+[M+H]+ 561.

9-Benzyl-8-(4-bromo-2-chlorophenyl)-6-(1-methylcyclopropoxy)-9H-purine was made following a similar procedure to 4-((8-(2-chloro-4-(2-(4-methylpiperazin-1-yl)ethoxy)phenyl)-6-(1-methylcyclopropoxy)-9H-purin-9-yl)methyl)-2-methylthiazole (Synthetic Example 7), using N-benzyl-6-(1-methylcyclopropoxy)-5-nitropyrimidin-4-amine (Synthetic Example 4, intermediate). and 4-bromo-2-chlorobenzaldehyde as starting materials. ¹H NMR (300 MHz, DMSO-d₆): 0.77-0.90 (2H, m), 0.90-1.12 (2H, m), 1.73 (3H, s), 5.32 (2H, s), 6.87-6.96 (2H, m), 7.15-7.26 (3H, m), 7.48 (1H, d), 7.70 (1H, dd), 7.96 (1H, d), 8.65 (1H, s). m/z: ES+[M+H]+ 469.

[M2] was synthesised from tert-butyl (1-(4-(9-benzyl-6-(1-methylcyclopropoxy)-9H-purin-8-yl)-3-chlorophenyl)piperidin-4-yl)carbamate through a BOC deprotection similar to the procedure used in Synthetic Example S1 to make 1-(3-(4-(9-benzyl-6-(1-methylcyclopropoxy)-9H-purin-8-yl)-3-chlorophenoxy)propyl)piperazin-2-one.

Tert-butyl (1-(4-(9-benzyl-6-(1-methylcyclopropoxy)-9H-purin-8-yl)-3-chlorophenyl)piperidin-4-yl)carbamate was synthesised from 9-benzyl-8-(4-bromo-2-chlorophenyl)-6-(1-methylcyclopropoxy)-9H-purine and tert-butyl piperidin-4-ylcarbamate by a palladium catalysed cross coupling similar to the synthesis of tert-butyl (1-(4-(9-benzyl-6-(1-methylcyclopropoxy)-9H-purin-8-yl)-3-chlorophenyl)azetidin-3-yl)carbamate (Synthetic Example M1, intermediate). ¹H NMR (300 MHz, DMSO-d₆): 0.78-0.89 (2H, m), 1.01 (2H, d), 1.39 (9H, s), 1.47 (2H, d), 1.73 (3H, s), 1.80 (2H, d), 2.91 (2H, t), 3.50 (1H, s), 3.82 (2H, d), 5.29 (2H, s), 6.92 (4H, ddt), 7.09 (1H, d), 7.18-7.27 (4H, m), 8.58 (1H, s). m/z: ES+[M+H]+ 589.

[M3] was synthesised through a palladium catalysed cross coupling as follows:

9-Benzyl-8-(4-bromo-2-chlorophenyl)-6-(1-methylcyclopropoxy)-9H-purine (100 mg, 0.21 mmol), piperazin-2-one (32.0 mg, 0.32 mmol), cesium carbonate (208 mg, 0.64 mmol) and rac-BINAP-Pd-G3 (44 mg, 0.04 mmol) in 1,4-dioxane (2 mL) were stirred under an atmosphere of nitrogen at 100° C. for 12 hours. The reaction mixture was filtered through celite, and the solvents evaporated. The crude product was purified by preparative HPLC (Waters CSH C18 OBD column, 5p silica, 30 mm diameter, 100 mm length), using decreasingly polar mixtures of water (containing 1% aqueous ammonia) and MeCN as eluents. Fractions containing the desired compound were evaporated to dryness to afford 4-(4-(9-benzyl-6-(1-methylcyclopropoxy)-9H-purin-8-yl)-3-chlorophenyl)piperazin-2-one (22.9 mg, 22%) as a pale yellow gum.

[M4] was synthesised using an ester hydrolysis from methyl (E)-3-(4-(9-benzyl-6-(1-methylcyclopropoxy)-9H-purin-8-yl)-3-chlorophenyl)acrylate as follows:

A solution of LiOH (65.5 mg, 2.74 mmol) in water (1 mL) was added to methyl (E)-3-(4-(9-benzyl-6-(1-methylcyclopropoxy)-9H-purin-8-yl)-3-chlorophenyl)acrylate (130 mg, 0.27 mmol) in EtOH (3 mL) at rt. The resulting mixture was stirred at rt for 1 hour. The reaction mixture was concentrated and adjusted to pH=3 with 3 M HCl and extracted with EtOAc (3×10 mL). The organic layer was dried over Na₂SO₄, filtered and evaporated. The crude product was purified by preparative HPLC; XBridge Shield RP18 OBD column, 30*150 mm, 5 μm; using decreasingly polar mixtures of water (containing 10 mmol/L NH₄HCO₃ and 0.1% aqueous ammonia) and MeCN as eluents. The fractions containing the desired compound were evaporated to dryness to afford (E)-3-(4-(9-benzyl-6-(1-methylcyclopropoxy)-9H-purin-8-yl)-3-chlorophenyl)acrylic acid (33 mg, 26%) as a white solid.

Methyl (E)-3-(4-(9-benzyl-6-(1-methylcyclopropoxy)-9H-purin-8-yl)-3-chlorophenyl)acrylate was made through a palladium catalysed cross coupling reaction as follows: PdCl₂(PPh₃)₂ (37.4 mg, 0.05 mmol) was added to tri(o-tolyl)phosphine (16.2 mg, 0.05 mmol), 9-benzyl-8-(4-bromo-2-chlorophenyl)-6-(1-methylcyclopropoxy)-9H-purine (250 mg, 0.53 mmol, Synthetic Example M1), methyl acrylate (55.0 mg, 0.64 mmol) and K₂CO₃ (81 mg, 0.59 mmol) in DMF (3 mL) was stirred at 120° C. for 1 hour. The reaction mixture was diluted with EtOAc (20 mL), and washed sequentially with saturated brine (3×75 mL). The organic layer was dried over Na₂SO₄, filtered and evaporated to afford crude product. The residue was purified by preparative TLC (petroleum ether: EtOAc=2:1), to afford methyl (E)-3-(4-(9-benzyl-6-(1-methylcyclopropoxy)-9H-purin-8-yl)-3-chlorophenyl)acrylate (0.14 g, 55%) as a colourless gum. ¹H NMR (400 MHz, DMSO-d₆): 0.86 (2H, d), 1.02 (2H, d), 1.74 (3H, s), 3.76 (3H, s), 5.33 (2H, s), 5.76 (1H, s), 6.90 (2H, q), 7.15-7.24 (3H, m), 7.57 (1H, d), 7.73 (1H, d), 7.83 (1H, d), 8.09 (1H, s), 8.65 (1H, s). m/z: ES+[M+H]+ 475.

[M5] was made from 9-benzyl-8-(4-bromo-2-chlorophenyl)-6-(1-methylcyclopropoxy)-9H-purine via a RockPhos coupling and subsequent BOC deprotection.

The RockPhos coupling to form tert-butyl 4-(2-(4-(9-benzyl-6-(1-methylcyclopropoxy)-9H-purin-8-yl)-3-chlorophenoxy)ethyl)-3-oxopiperazine-1-carboxylate from 9-benzyl-8-(4-bromo-2-chlorophenyl)-6-(1-methylcyclopropoxy)-9H-purine (Synthetic Example M1 intermediate) and tert-butyl 4-(2-hydroxyethyl)-3-oxopiperazine-1-carboxylate followed a similar procedure to the synthesis of 4-methyl-6-(2-(4-methylpiperazin-1-yl)ethoxy)nicotinaldehyde (Synthetic Example A5, intermediate). The product of this reaction was used directly in the deprotection without further purification. m/z: ES+[M+H]+ 633.

The BOC deprotection of tert-butyl 4-(2-(4-(9-benzyl-6-(1-methylcyclopropoxy)-9H-purin-8-yl)-3-chlorophenoxy)ethyl)-3-oxopiperazine-1-carboxylate was done using similar conditions previously described in crude tert-butyl 4-(3-(4-(9-benzyl-6-(1-methylcyclopropoxy)-9H-purin-8-yl)-3-chlorophenoxy)propyl)-3-oxopiperazine-1-carboxylate (Synthetic Example S1, intermediate).

The following examples in Table N were synthesised according the notes at the bottom of Table N.

TABLE N

| | | | | LCMS |
|---|---|---|---|---|
| Ex# | Structure | Name | ¹H NMR | [M + H]⁺ |
| N1 | 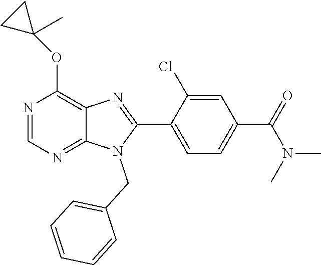 [SMILES: CN(C)C(=O)c1ccc(-c2nc3c(OC4(C)CC4)ncnc3n2Cc2ccccc2)c(Cl)c1] | 4-(9-benzyl-6- (1- methylcyclopro- poxy)-9H- purin-8-yl)-3- chloro-N,N- dimethylbenz- amide | (300 MHz, DMSO-d₆): 0.79- 0.88 (2H, m), 1.02 (2H, d), 1.74 (3H, s), 2.93 (3H, s), 3.01 (3H, s), 5.34 (2H, s), 6.89 (2H, dd), 7.18 (3H, dd), 7.45 (1H, dd), 7.55 (1H, d), 7.66 (1H, d), 8.66 (1H, s) | 462 |
| N2 | 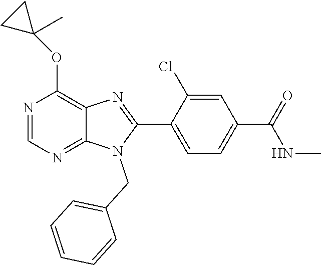 [SMILES: CNC(=O)c1ccc(-c2nc3c(OC4(C)CC4)ncnc3n2Cc2ccccc2)c(Cl)c1] | 4-(9-benzyl-6- (1- methylcyclopro- poxy)-9H- purin-8-yl)-3- chloro-N- methylbenza- mide | (400 MHz, DMSO-d₆): 0.86 (2H, d), 1.02 (2H, d), 1.74 (3H, s), 2. 82 (3H, d), 5.32 (2H, s), 6.83-6.93 (2H, m), 7.19 (3H, m), 7.63 (1H, d), 7.88 (1H, dd), 8.04 (1H, d), 8.66 (1H, s), 8.73 (1H, d) | 448 |
| N3 | 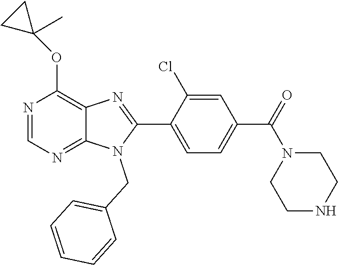 [SMILES: CC1(Oc2ncnc3c2nc(-c2ccc(C(=O)N4CCNCC4)cc2Cl)n3Cc2ccccc2)CC1] | (4-(9-benzyl- 6-(1- methylcyclopro- poxy)-9H- purin-8-yl)-3- chlorophenyl) (piperazin-1- yl)methanone | (400 MHz, DMSO-d₆): 0.80- 0.87 (2H, m), 1.01 (2H, d), 1.73 (3H, s), 2.72 (4H, d), 3.24 (2H, s), 3.57 (2H, s), 5.33 (2H, s), 6.83- 6.91 (2H, m), 7.11-7.22 (3H, m), 7.41 (1H, dd), 7.53 (1H, d), 7.61 (1H, d), 8.64 (1H, s). One proton not observed. | 503 |
| N4 | 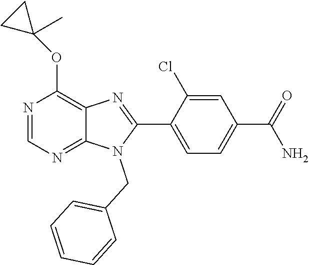 [SMILES: CC1(Oc2ncnc3c2nc(-c2ccc(C(N)=O)cc2Cl)n3Cc2ccccc2)CC1] | 4-(9-benzyl-6- (1- methylcyclopro- poxy)-9H- purin-8-yl)-3- chlorobenzamide | (300 MHz, DMSO-d₆): 0.85 (2H, t), 1.03 (2H, t), 1.73 (3H, s), 5.32 (2H, s), 6.88- 6.90 (2H, m), 7.18-7.21 (3H, m), 7.62 (1H, d), 7.72 (1H, s), 7.91-7. 93 (1H, m), 8.09 (1H, d), 8.24 (1H, s), 8.66 (1H, s). | 434 |
| N5 | 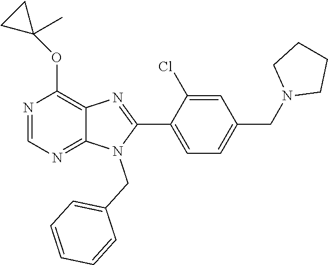 [SMILES: CC1(Oc2ncnc3c2nc(-c2ccc(CN4CCCC4)cc2Cl)n3Cc2ccccc2)CC1] | 9-benzyl-8-(2- chloro-4- (pyrrolidin-1- ylmethyl)phe- nyl)-6-(1- methylcyclopro- poxy)-9H- purine | (300 MHz, DMSO-d₆): 0.80- 0.89 (2H, m), 1.02 (2H, d), 1.73 (7H, d), 2.46 (4H, d), 3.66 (2H, s), 5.30 (2H, s), 6.76-6.97 (2H, m), 7.12-7.25 (3H, m), 7.37 (1H, dd), 7. 42 (1H, d), 7.55 (1H, d), 8.64 (1H, s) | 474 |
| N6 | 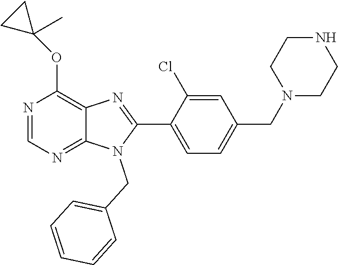 [SMILES: CC1(Oc2ncnc3c2nc(-c2ccc(CN4CCNCC4)cc2Cl)n3Cc2ccccc2)CC1] | 9-benzyl-8-(2- chloro-4- (piperazin-1- ylmethyl)phe- nyl)-6-(1- methylcyclopro- poxy)-9H- purine | (300 MHz, DMSO-d₆): 0.80- 0.88 (2H, m), 1.02 (2H, d), 1.73 (3H, s), 2.34 (4H, s), 2.75 (4H, t), 3.53 (2H, s), 5.30 (2H, s), 6.80- 6.89 (2H, m), 7.13-7.22 (3H, m), 7.33-7.46 (2H, m), 7.54 (1H, d), 8.64 (1H, s). One proton not observed. | 489 |
| N7 | 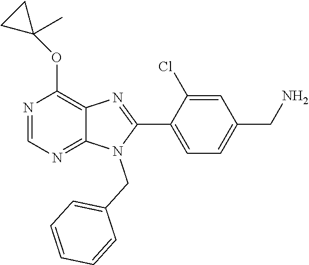 [SMILES: CC1(Oc2ncnc3c2nc(-c2ccc(CN)cc2Cl)n3Cc2ccccc2)CC1] | (4-(9-benzyl- 6-(1- methylcyclopro- poxy)-9H- purin-8-yl)-3- chlorophenyl) methanamine | (300 MHz, DMSO-d₆): 0.84 (2H, t), 1.02 (2H, t), 1.72 (3H, s), 3.96 (2H, s), 5.29 (2H, s), 6.89 (2H, t), 7.20 (3H, t), 7.47-7.54 (2H, m), 7.72 (1H, s), 8.63 (1H, s). Two protons not observed. | 420 |
| N8 | 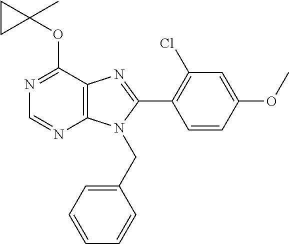 [SMILES: COc1ccc(-c2nc3c(OC4(C)CC4)ncnc3n2Cc2ccccc2)c(Cl)c1] | 9-benzyl-8-(2- chloro-4- methoxyphenyl)- 6-(1- methylcyclopro- poxy)-9H- purine | (500 MHz, DMSO-d₆): 0.81- 0.87 (2H, m), 0. 99- 1.04 (2H, m), 1.73 (3H, s), 3.86 (3H, s), 5.29 (2H, s), 6.87-6.92 (2H, m), 7.03 (1H, dd), 7.18-7.22 (3H, m), 7.23 (1H, d), 7.42 (1H, d), 8.61 (1H, s). | 421 |
| N9 | 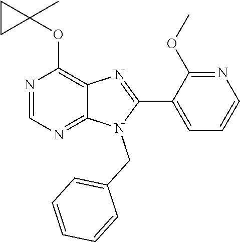 [SMILES: COc1ncccc1-c1nc2c(OC3(C)CC3)ncnc2n1Cc1ccccc1] | 9-benzyl-8-(2- methoxypyridin- 3-yl)-6-(1- methylcyclopro- poxy)-9H- purine | (500 MHz, CDCl₃): 0.79- 0.86 (2H, m), 1.12-1.18 (2H, m), 1.79 (3H, s), 3.86 (3H, s), 5.39 (2H, s), 6.89- 6.96 (3H, m), 7.16 (3H, dd), 7.66 (1H, dd), 8.30 (1H, dd), 8.66 (1H, s). | 388 |

[N1] was synthesised through amide coupling reaction from 4-(9-benzyl-6-(1-methylcyclopropoxy)-9H-purin-8-yl)-3-chlorobenzoic acid and dimethylamine through the following procedure:

T3P (658 mg, 1.03 mmol) was added to 4-(9-benzyl-6-(1-methylcyclopropoxy)-9H-purin-8-yl)-3-chlorobenzoic acid (150 mg, 0.34 mmol), dimethylamine (345 μL, 0.69 mmol, 2M in THF) and DIEA (181 μL, 1.03 mmol) in DMA (2 mL) at 0° C. for 15 minutes. The resulting mixture was stirred at rt for 1 hour. The solvent was removed under reduced pressure. The crude product was purified by preparative HPLC, XBridge Prep OBD C18 column, 30*150 mm, 5 μm; using decreasingly polar mixtures of water (containing 10 mmol/L NH₄HCO₃ and 0.1% aqueous ammonia) and MeCN as eluents. The fractions containing the desired compound were evaporated to dryness to afford 4-(9-benzyl-6-(1-methylcyclopropoxy)-9H-purin-8-yl)-3-chloro-N,N-dimethylbenzamide (0.042 g, 26%) as a white solid.

4-(9-Benzyl-6-(1-methylcyclopropoxy)-9H-purin-8-yl)-3-chlorobenzoic acid was made following a similar ester hydrolysis procedure to (E)-3-(4-(9-benzyl-6-(1-methylcyclopropoxy)-9H-purin-8-yl)-3-chlorophenyl)acrylic acid (Synthetic Example M4), using methyl 4-(9-benzyl-6-(1-methylcyclopropoxy)-9H-purin-8-yl)-3-chlorobenzoate as starting material. ¹H NMR (300 MHz, DMSO-d₆): 0.78-0.90 (2H, m), 1.03 (2H, d), 1.74 (3H, s), 5.33 (2H, s), 6.89 (2H, dd), 7.19 (3H, dd), 7.66 (1H, d), 7.96 (1H, dd), 8.07 (1H, d), 8.67 (1H, s). One proton not observed. m/z: ES+[M+H]+ 435.

Methyl 4-(9-benzyl-6-(1-methylcyclopropoxy)-9H-purin-8-yl)-3-chlorobenzoate was made via a cyclisation reaction similar to a procedure previously described in the synthesis of 4-(1-benzyl-5-isopropoxy-1H-benzo[d]imidazol-2-yl)-3-chlorophenol (Synthetic Example A1), using N-benzyl-6-(1-methylcyclopropoxy)-5-nitropyrimidin-4-amine (Synthetic Example 4, intermediate) and methyl 3-chloro-4-formylbenzoate as starting materials. ¹H NMR (300 MHz, DMSO-d₆): 0.78-0.92 (2H, m), 1.02 (2H, d), 1.74 (3H, s), 3.92 (3H, s), 5.34 (2H, s), 6.80-6.95 (2H, m), 7.19 (3H, m), 7.69 (1H, d), 7.99 (1H, dd), 8.09 (1H, d), 8.68 (1H, s). m/z: ES+[M+H]+ 449.

[N2] was synthesised through amide coupling reaction similar to the previously described procedure for the synthesis of 4-(9-benzyl-6-(1-methylcyclopropoxy)-9H-purin-8-yl)-3-chloro-N,N-dimethylbenzamide, using 4-(9-benzyl-6-(1-methylcyclopropoxy)-9H-purin-8-yl)-3-chlorobenzoic acid (Synthetic Example N1, intermediate) and methanamine as starting materials.

[N3] was synthesised through amide coupling reaction similar to the previously described procedure for the synthesis of 4-(9-benzyl-6-(1-methylcyclopropoxy)-9H-purin-8-yl)-3-chloro-N,N-dimethylbenzamide, using 4-(9-benzyl-6-(1-methylcyclopropoxy)-9H-purin-8-yl)-3-chlorobenzoic acid (Synthetic Example N1, intermediate) and piperazine as starting material.

[N4] was synthesised through amide coupling reaction similar to the previously described procedure for the synthesis of 4-(9-benzyl-6-(1-methylcyclopropoxy)-9H-purin-8-yl)-3-chloro-N,N-dimethylbenzamide, using 4-(9-benzyl-6-(1-methylcyclopropoxy)-9H-purin-8-yl)-3-chlorobenzoic acid (Synthetic Example N1, intermediate) and ammonium chloride as starting material.

[N5] was made via a substitution reaction from 4-(9-benzyl-6-(1-methylcyclopropoxy)-9H-purin-8-yl)-3-chlorobenzyl methanesulfonate and pyrrolidine as follows:

Pyrrolidine (34.2 mg, 0.48 mmol) was added to 4-(9-benzyl-6-(1-methylcyclopropoxy)-9H-purin-8-yl)-3-chlorobenzyl methanesulfonate (120 mg, 0.24 mmol) and K₂CO₃ (100 mg, 0.72 mmol) in DMA (2 mL) at rt. The resulting mixture was stirred at 80° C. for 1 hour. The reaction mixture was diluted with water (25 mL), and washed sequentially with EtOAc (3×25 mL) and saturated brine (25 mL). The organic layer was dried over Na₂SO₄, filtered and evaporated to afford crude product. The crude product was purified by preparative HPLC, XBridge Prep OBD C18 column, 30*150 mm, 5 m; using decreasingly polar mixtures of water (containing 10 mmol/L NH₄HCO₃ and 0.1% aqueous ammonia) and MeCN as eluents. The fractions containing the desired compound were evaporated to dryness to afford 9-benzyl-8-(2-chloro-4-(pyrrolidin-1-ylmethyl)phenyl)-6-(1-methylcyclopropoxy)-9H-purine (0.012 g, 10.88%) as a white solid.

4-(9-Benzyl-6-(1-methylcyclopropoxy)-9H-purin-8-yl)-3-chlorobenzyl methanesulfonate was made as follows:

Ms₂O (149 mg, 0.86 mmol) in DCM (2 mL) was added dropwise into the solution of (4-(9-benzyl-6-(1-methylcyclopropoxy)-9H-purin-8-yl)-3-chlorophenyl)methanol (300 mg, 0.71 mmol) and TEA (216 mg, 2.14 mmol) in DCM (10 mL) at rt. The resulting mixture was stirred at rt for 2 hour. The reaction mixture was redissolved in DCM (50 mL), and washed sequentially with water (2×10 mL) and saturated brine (10 mL). The organic layer was dried over Na₂SO₄, filtered and evaporated to afford crude 4-(9-benzyl-6-(1-methylcyclopropoxy)-9H-purin-8-yl)-3-chlorobenzyl methanesulfonate (300 mg). The crude product was used to next directly without future purification.

(4-(9-Benzyl-6-(1-methylcyclopropoxy)-9H-purin-8-yl)-3-chlorophenyl)methanol was made through an ester reduction of methyl 4-(9-benzyl-6-(1-methylcyclopropoxy)-9H-purin-8-yl)-3-chlorobenzoate as follows:

A solution of LiAlH₄ (0.61 mL, 1.23 mmol, 2.4 M in THF) was added dropwise to methyl 4-(9-benzyl-6-(1-methylcyclopropoxy)-9H-purin-8-yl)-3-chlorobenzoate (Synthetic Example N1, intermediate; 1.1 g, 2.45 mmol) in THF (10 mL) at 0° C. under nitrogen. The resulting mixture was stirred at rt for 1 hour. The reaction mixture was poured into water and 15% aq. NaOH. The organic layer was dried over Na₂SO₄, filtered and evaporated to afford (4-(9-benzyl-6-(1-methylcyclopropoxy)-9H-purin-8-yl)-3-chlorophenyl)methanol (0.90 g, 87%) as a yellow solid. The product was used in the next step directly without further purification. ¹H NMR (300 MHz, DMSO-d₆): 0.78-0.90 (2H, m), 0.97-1.08 (2H, m), 1.74 (3H, s), 4.61 (2H, d), 5.30 (2H, s), 5.50 (1H, t), 6.90 (2H, dt), 7.20 (3H, ddt), 7.40 (1H, dd), 7.48 (1H, d), 7.58 (1H, d), 8.63 (1H, s). m/z: ES+[M+H]+ 421.

[N6] was made via a reductive amination from 4-(9-benzyl-6-(1-methylcyclopropoxy)-9H-purin-8-yl)-3-chlorobenzaldehyde as follows:

Piperazine (103 mg, 1.19 mmol) was added to 4-(9-benzyl-6-(1-methylcyclopropoxy)-9H-purin-8-yl)-3-chlorobenzaldehyde (100 mg, 0.24 mmol) in DCM (4 mL) at rt. After 30 minutes, sodium triacetoxyborohydride (253 mg, 1.19 mmol) was added. The resulting mixture was stirred at rt for 1 hour. The reaction was quenched with MeOH (2 mL), concentrated under vacuum to afford a crude product. The crude product was purified by preparative HPLC, XBridge Prep OBD C18 column, 30*150 mm, 5 m; using decreasingly polar mixtures of water (containing 10 mmol/L NH₄HCO₃ and 0.1% aqueous ammonia) and MeCN as eluents. Fractions containing the desired compound were evaporated to dryness to afford 9-benzyl-8-(2-chloro-4-(piperazin-1-ylmethyl)phenyl)-6-(1-methylcyclopropoxy)-9H-purine (10 mg, 9%) as a white solid.

4-(9-Benzyl-6-(1-methylcyclopropoxy)-9H-purin-8-yl)-3-chlorobenzaldehyde was formed through the oxidation of (4-(9-benzyl-6-(1-methylcyclopropoxy)-9H-purin-8-yl)-3-chlorophenyl)methanol (Synthetic Example N5, intermediate) as follows:

Manganese(IV) oxide (640 mg, 4.28 mmol) was added to (4-(9-benzyl-6-(1-methylcyclopropoxy)-9H-purin-8-yl)-3-chlorophenyl)methanol (600 mg, 1.43 mmol) in EtOAc (15 mL) at rt. The resulting mixture was stirred at 80° C. for 16 hours. The reaction mixture was filtered through celite. The solvent was removed under reduced pressure. The crude product was purified by flash silica chromatography, elution gradient 0 to 50% EtOAc in pentane. Pure fractions were evaporated to dryness to afford 4-(9-benzyl-6-(1-methylcyclopropoxy)-9H-purin-8-yl)-3-chlorobenzaldehyde (0.450 g, 75%) as a yellow solid.

¹H NMR (300 MHz, DMSO-d₆): 0.81-0.89 (2H, m), 1.03 (2H, d), 1.74 (3H, s), 5.35 (2H, s), 6.85-6.93 (2H, m), 7.14-7.24 (3H, m), 7.77 (1H, d), 7.97 (1H, dd), 8.14 (1H, d), 8.68 (1H, s), 10.09 (1H, s). m/z: ES+[M+H]+ 419.

[N7] was made via the reduction of (E)-4-(9-benzyl-6-(1-methylcyclopropoxy)-9H-purin-8-yl)-3-chlorobenzaldehyde oxime as follows:

Zinc (24.9 mg, 0.38 mmol) was added to (E)-4-(9-benzyl-6-(1-methylcyclopropoxy)-9H-purin-8-yl)-3-chlorobenzaldehyde oxime (55 mg, 0.13 mmol) in acetic acid (0.5 mL) at rt. The resulting mixture was stirred at rt for 1 hour. The solids were filtered off and the filtrate was concentrated under reduced pressure. The crude product was purified by preparative HPLC, Xselect CSH C18 OBD column; 30*150 mm, 5 μm; using decreasingly polar mixtures of water (containing 0.1% formic acid) and MeCN as eluents. The fractions containing the desired compound were evaporated to dryness to afford (4-(9-benzyl-6-(1-methylcyclopropoxy)-9H-purin-8-yl)-3-chlorophenyl)methanamine (14 mg, 26%) as a white solid.

(E)-4-(9-benzyl-6-(1-methylcyclopropoxy)-9H-purin-8-yl)-3-chlorobenzaldehyde oxime was made as follows:

Na₂CO₃ (29.6 mg, 0.28 mmol) was added to 4-(9-benzyl-6-(1-methylcyclopropoxy)-9H-purin-8-yl)-3-chlorobenzaldehyde (Synthetic Example N6, intermediate) (65 mg, 0.16 mmol) and hydroxylammonium chloride solution (16.2 mg, 0.23 mmol) in water (1 mL)/MeOH (0.400 mL) at rt. The resulting mixture was stirred at rt for 3 hours. The solvent was removed under reduced pressure. The reaction mixture was diluted with EtOAc (10 mL), and washed sequentially with water (2×5 mL) and saturated brine (10 mL). The organic layer was dried over Na₂SO₄, filtered and evaporated to afford (E)-4-(9-benzyl-6-(1-methylcyclopropoxy)-9H-purin-8-yl)-3-chlorobenzaldehyde oxime (0.060 g, 89%) as yellow gum, which was used without any further purification. ¹H NMR (300 MHz, DMSO-d₆): 0.85 (2H, t), 1.03 (2H, t), 1.74 (3H, s), 5.34 (2H, s), 6.85-6.96 (3H, m), 7.18-7.24 (3H, m), 7.9-8.3 (2H, m), 8.66 (1H, s), 10.09 (1H, s), 11.72 (1H, s). m/z: ES+[M+H]+ 434.

[N8]9-Benzyl-8-(2-chloro-4-methoxyphenyl)-6-(1-methylcyclopropoxy)-9H-purine was made as follows:

9-Benzyl-8-bromo-6-(1-methylcyclopropoxy)-9H-purine (123 mg, 0.34 mmol, see Synthetic Example 5 intermediate), (2-chloro-4-methoxyphenyl)boronic acid (70 mg, 0.38 mmol), cesium carbonate (223 mg, 0.68 mmol) and tetrakis(triphenylphosphine)palladium(0) (19.8 mg, 0.02 mmol) were dissolved in 1,4-dioxane (1 mL) and water (0.2 mL) and sealed into a microwave tube. The reaction was heated to 110° C. for 2 hours in the microwave reactor and cooled to RT. The reaction mixture was diluted with EtOAc (50 mL), washed with water (50 mL), brine (50 mL), dried over MgSO₄, filtered and concentrated under reduced pressure. The crude sample was dissolved in DMSO, then purified by preparative HPLC (Waters CSH C18 OBD, 30×100 mm, 5 μm), using decreasingly polar mixtures of H20 (containing 0.3% ammonium hydroxide (aq)) and MeCN as eluents. After chromatography, fractions containing the desired compound were concentrated under reduced pressure to afford 9-benzyl-8-(2-chloro-4-methoxyphenyl)-6-(1-methylcyclopropoxy)-9H-purine (106 mg, 74%) as a colourless film. ¹H NMR (500 MHz, DMSO-d₆): 0.81-0.87 (2H, m), 0.99-1.04 (2H, m), 1.73 (3H, s), 3.86 (3H, s), 5.29 (2H, s), 6.87-6.92 (2H, m), 7.03 (1H, dd), 7.18-7.22 (3H, m), 7.23 (1H, d), 7.42 (1H, d), 8.61 (1H, s). m/z: ES+[M+H]+ 421.

[N9]9-benzyl-8-(2-methoxypyridin-3-yl)-6-(1-methylcyclopropoxy)-9H-purine was made as follows:

To a stirred solution of 3-(9-benzyl-6-(1-methylcyclopropoxy)-9H-purin-8-yl)pyridin-2(1H)-one (38.7 mg, 0.10 mmol) and potassium carbonate (28.2 mg, 0.20 mmol) in DMF (0.5 mL), was added iodomethane (17.2 mg, 0.12 mmol). The reaction stirred at RT for 21 hrs. The reaction mixture then filtered and concentrated in vacuo. The crude product was purified by preparative HPLC (Waters CSH C18 OBD column, 5 μm silica, 30 mm diameter, 100 mm length), using decreasingly polar mixtures of water (containing 1% of aq. ammonia)) and MeCN as eluents. Fractions containing the desired compound were evaporated to dryness to yield 9-benzyl-8-(2-methoxypyridin-3-yl)-6-(1-methylcyclopropoxy)-9H-purine (1.2 mg, 3%).

3-(9-Benzyl-6-(1-methylcyclopropoxy)-9H-purin-8-yl)pyridin-2(1H)-one was made starting from 9-benzyl-8-bromo-6-(1-methylcyclopropoxy)-9H-purine (Synthetic Example 5 intermediate) and (2-oxo-1,2-dihydropyridin-3-yl)boronic acid in a similar procedure as described for 4-(9-benzyl-6-(1-methylcyclopropoxy)-9H-purin-8-yl)-3-chlorophenol (see Synthetic Example 5). ¹H NMR (500 MHz, DMSO-d₆): 0.81-0.86 (2H, m), 0.98-1.03 (2H, m), 1.72 (3H, s), 5.54 (2H, s), 6.30 (1H, t), 6.9-6.96 (2H, m), 7.13-7.21 (3H, m), 7.64 (1H, dd), 7.72 (1H, dd), 8.58 (1H, s), 12.25 (1H, s). m/z: ES+[M+H]+ 374

The following examples in Table P were synthesised according the notes at the bottom of Table P.

TABLE P

| | | | | LCMS |
|---|---|---|---|---|
| Ex# | Structure | Name | ¹H NMR | [M + H]⁺ |
| P1 | 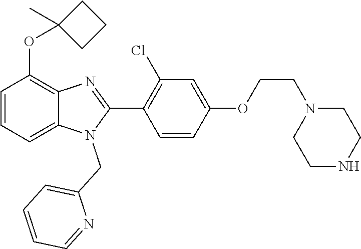 [SMILES: CC1(Oc2cccc3c2nc(-c2ccc(OCCN4CCNCC4)cc2Cl)n3Cc2ccccn2)CCC1] | 2-(2-chloro-4-(2- (piperazin-1- yl)ethoxy)phenyl)- 4-(1- methylcyclobutoxy)- 1-(pyridin-2- ylmethyl)-1H- benzo[d]imidazole | (300 MHz, DMSO-d₆): 1.60 (3H, s), 1.67-1.83 (2H, m), 2.17-2.29 (2H, m), 2.35-2.48 (6H, m), 2.63- 2.71 (6H, m), 4.16 (2H, t), 5.32 (2H, s), 6.55 (1H, dd), 6.90-7.13 (4H, m), 7.19-7.29 (2H, m), 7.44 (1H, d), 7.68 (1H, td), 8.35- 8.49 (1H, m). One proton not observed. | 532 |
| P2 | 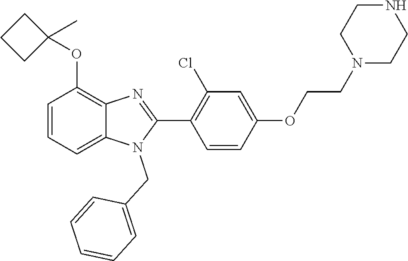 [SMILES: CC1(Oc2cccc3c2nc(-c2ccc(OCCN4CCNCC4)cc2Cl)n3Cc2ccccc2)CCC1] | 1-benzyl-2-(2- chloro-4-(2- (piperazin-1- yl)ethoxy)phenyl)- 4-(1- methylcyclobutoxy)- 1H- benzo[d]imidazole | (400 MHz, DMSO-d₆): 1.59 (3H, s), 1.7-1.79 (2H, m), 2.22 (2H, t), 2.36-2.47 (6H, m), 2.66-2.70 (6H, m), 4.18 (2H, t), 5.25 (2H, s), 6.55 (1H, d), 6.95-7.1 (5H, m), 7.18-7.28 (4H, m), 7.47 (1H, d). One proton not observed. | 531 |
| P3 | 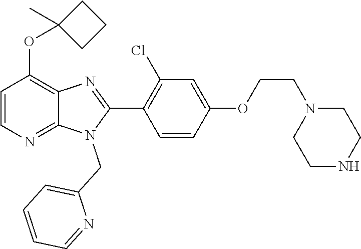 [SMILES: CC1(Oc2ccnc3c2nc(-c2ccc(OCCN4CCNCC4)cc2Cl)n3Cc2ccccn2)CCC1] | 2-(2-chloro-4-(2- (piperazin-1- yl)ethoxy)phenyl)- 7-(1- methylcyclobutoxy)- 3-(pyridin-2- ylmethyl)-3H- imidazo[4,5- b]pyridine | (400 MHz, CDCl₃) 1.75-1.85 (5H, m), 1.88-2 (2H, m), 2.29-2.42 (2H, m), 2.62- 2.81 (3H, m), 2.93-3.1 (4H, m), 3.2-3.39 (4H, m), 4.02-4.3 (2H, m), 5.52 (2H, s), 6.59 (1H, d), 6.80- 6.86 (2H, m), 6.96-7.04 (1H, m), 7.07-7.16 (1H, m), 7.31 (1H, d), 7.45-7.58 (1H, m), 8.23 (1H, d), 8.43 (1H, d). | 533 |
| P4 | 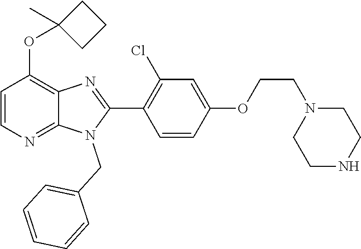 [SMILES: CC1(Oc2ccnc3c2nc(-c2ccc(OCCN4CCNCC4)cc2Cl)n3Cc2ccccc2)CCC1] | 3-benzyl-2-(2- chloro-4-(2- (piperazin-1- yl)ethoxy)phenyl)- 7-(1- methylcyclobutoxy)- 3H- imidazo[4,5- b]pyridine | (400 MHz, CDCl₃): 1.72 (5H, s), 1.83-1.95 (2H, m), 2.23-2.37 (3H, m), 2.55- 2.74 (4H, m), 2.83-2.91 (2H, m), 3.04-3.14 (3H, m), 4.12 (2H, t), 5.36 (2H, s), 6.54 (1H, d), 6.77 (1H, dd), 6.86-6.96 (2H, m), 7.02 (1H, d), 7.11-7.17 (3H, m), 7.20 (1H, d), 8.22 (1H, d). One proton not observed. | 532 |

p-Toluenesulfonic acid (58.3 mg, 0.34 mmol) was added to 3-(1-methylcyclobutoxy)-N1-(pyridin-2-ylmethyl)benzene-1,2-diamine (64.0 mg, 0.23 mmol) and 2-chloro-4-(2-(piperazin-1-yl)ethoxy)benzaldehyde (72.8 mg, 0.27 mmol, Synthetic Example 14 intermediate) in EtOH (2 mL) at 20° C. The resulting mixture was stirred at 25° C. for 2 hours. The reaction mixture was adjusted to pH=8 with saturated NaHCO₃. The reaction mixture was concentrated and diluted with EtOAc (20 mL) and washed sequentially with water (3×5 mL), saturated brine (3×5 mL). The top layer was dried over Na₂SO₄, filtered and evaporated to afford crude product. The crude product was purified by preparative HPLC (Column: XBridge Prep Phenyl OBD Column, 19*250 mm, 5 μm), using decreasingly polar mixtures of water (containing 10 mmol/L NH₄HCO₃ and 0.1% aqueous ammonia) and MeCN as eluents. Fractions containing the desired compound were evaporated to dryness and purified further by SFC (Column:YMC-Triart Diol-HILIC, 100*4.6 mm, 3 m), eluting with 90% scCO2, and MeOH (containing 1% 2M NH₃-MeOH). Fractions containing the desired compound were evaporated to dryness to afford 2-(2-chloro-4-(2-(piperazin-1-yl)ethoxy)phenyl)-4-(1-methylcyclobutoxy)-1-(pyridin-2-ylmethyl)-1H-benzo[d]imidazole (7.7 mg, 6.4%) as a white solid.

3-(1-Methylcyclobutoxy)-N1-(pyridin-2-ylmethyl)benzene-1,2-diamine was made following a similar procedure to N4-benzyl-2-isopropoxypyridine-3,4-diamine (Synthetic Example 9, intermediate), using 3-(1-methylcyclobutoxy)-2-nitro-N-(pyridin-2-ylmethyl)aniline as the starting material. ¹H NMR (400 MHz, CDCl₃): 1.28 (1H, d), 1.58 (3H, s), 1.63-1.87 (2H, m), 2.15 (2H, ddt), 2.4-2.54 (2H, m), 3.07 (4H, s), 6.33 (2H, d), 6.66 (1H, t), 7.24 (1H, d), 7.41 (1H, d), 7.72 (1H, td), 8.58-8.66 (1H, m). m/z: ES+[M+H]+ 284.

3-(1-Methylcyclobutoxy)-2-nitro-N-(pyridin-2-ylmethyl)aniline was made as follows:

1-Fluoro-3-(1-methylcyclobutoxy)-2-nitrobenzene (300 mg, 1.33 mmol),

pyridin-2-ylmethanamine (173 mg, 1.60 mmol) and potassium carbonate (552 mg, 4.00 mmol) was added in DMF (5 mL) at 20° C. The resulting mixture was stirred at 100° C. for 16 hours. The reaction mixture was concentrated and diluted with EtOAc (3×20 mL), and washed sequentially with water (75 mL), saturated brine (2×15 mL). The organic layer was dried over Na₂SO₄, filtered and evaporated to afford crude product. The residue was purified by preparative TLC (petroleum ether:EtOAc, 2:1), to afford 3-(1-methylcyclobutoxy)-2-nitro-N-(pyridin-2-ylmethyl)aniline (220 mg, 53%) as an orange oil. ¹H NMR (400 MHz, DMSO-d₆): 1.50 (3H, s), 1.6-1.85 (2H, m), 2.07-2.21 (2H, m), 2.21-2.39 (2H, m), 4.45 (2H, d), 6.18 (1H, dd), 6.23-6.38 (1H, m), 6.79 (1H, t), 7.13 (1H, t), 7.24-7.3 (1H, m), 7.32 (1H, d), 7.77 (1H, td), 8.54 (1H, dt). m/z: ES+[M+H]+ 314.

1-Fluoro-3-(1-methylcyclobutoxy)-2-nitrobenzene was made following a similar procedure to 9-benzyl-6-(1-methylcyclobutoxy)-9H-purine (Synthetic Example 12, intermediate), using 1-methylcyclobutan-1-ol and 1,3-difluoro-2-nitrobenzene as starting materials. ¹H NMR (300 MHz, CDCl₃): 1.21-1.34 (2H, m), 1.57 (3H, s), 2.12-2.23 (2H, m), 2.4-2.52 (2H, m), 6.65 (1H, ddd), 6.77 (1H, ddd), 7.29 (1H, ddd).

[P2] was made as follows:

Ferric chloride solution (43.1 mg, 0.27 mmol) was added to N1-benzyl-3-(1-methylcyclobutoxy)benzene-1,2-diamine (50 mg, 0.18 mmol) and 2-chloro-4-(2-(piperazin-1-yl)ethoxy)benzaldehyde (95 mg, 0.35 mmol, Synthetic Example 14 intermediate) in 2-propanol (5 mL) at 25° C. The resulting mixture was stirred at 80° C. for 30 minutes. The reaction mixture was basified with saturated aq. NaHCO₃. The reaction mixture was concentrated and diluted with EtOAc (25 mL) and washed sequentially with water (2×10 mL) and saturated brine (2×10 mL). The organic layer was dried over Na₂SO₄, filtered and evaporated to afford crude product. The crude product was purified by flash C18-flash chromatography, elution gradient 0 to 80% AcOH in water. Fractions were evaporated to dryness to afford crude product. The crude product was purified by preparative HPLC (XBridge Prep C18 OBD column, 19*250 mm, 5 μm silica) using decreasingly polar mixtures of water (containing 10 mmol/L NH₄HCO₃ and 0.1% aq. ammonia) and MeCN as eluents. Fractions containing the desired compound were evaporated to dryness to afford 1-benzyl-2-(2-chloro-4-(2-(piperazin-1-yl)ethoxy)phenyl)-4-(1-methylcyclobutoxy)-1H-benzo[d]imidazole (5.7 mg, 6%) as a white solid.

N1-Benzyl-3-(1-methylcyclobutoxy)benzene-1,2-diamine was made following a similar procedure to 3-(1-methylcyclobutoxy)-N1-(pyridin-2-ylmethyl)benzene-1,2-diamine (Synthetic Example P1, intermediate), using N-benzyl-3-(1-methylcyclobutoxy)-2-nitroaniline as the starting material. m/z: ES+[M+H]+ 283.

N-Benzyl-3-(1-methylcyclobutoxy)-2-nitroaniline was made following a similar procedure to 3-(1-methylcyclobutoxy)-2-nitro-N-(pyridin-2-ylmethyl)aniline (Synthetic Example P1, intermediate), using 1-fluoro-3-(1-methylcyclobutoxy)-2-nitrobenzene (Synthetic Example P1, intermediate) and phenylmethanamine as starting materials. ¹H NMR (400 MHz, DMSO-d₆): 1.48 (3H, s), 1.68-1.8 (2H, m), 2.05-2.18 (2H, m), 2.22-2.31 (2H, m), 4.36 (2H, d), 6.13 (1H, d), 6.24 (1H, d), 6.64 (1H, t), 7.06 (1H, t), 7.18-7.27 (1H, m), 7.3-7.34 (3H, m). One proton not observed. m/z: ES+[M+H]+ 313.

[P3] was synthesised as follows:

4-Methylbenzoic acid (32 mg, 0.24 mmol) was added to 4-(1-methylcyclobutoxy)-N2-(pyridin-2-ylmethyl)pyridine-2,3-diamine (45 mg, 0.16 mmol) and 2-chloro-4-(2-(piperazin-1-yl)ethoxy)benzaldehyde (85 mg, 0.32 mmol) in EtOH (2 mL) 25° C. The resulting mixture was stirred at 80° C. for 4 hours. The reaction mixture was adjusted to pH=8 with saturated aq. NaHCO₃. The reaction mixture was concentrated and diluted with EtOAc (20 mL), and washed sequentially with water (3×10 mL), saturated brine (3×5 mL). The top layer was dried over Na₂SO₄, filtered and evaporated to afford crude product. The crude product was purified by preparative HPLC (XBridge Prep OBD column, 30*150 mm, 5 μm), using decreasingly polar mixtures of water (containing 10 mmol/L NH₄HCO₃ and 0.1% aq. ammonia) and MeCN as eluents. Fractions containing the desired compound were evaporated to dryness to afford 2-(2-chloro-4-(2-(piperazin-1-yl)ethoxy)phenyl)-7-(1-methylcyclobutoxy)-3-(pyridin-2-ylmethyl)-3H-imidazo[4,5-b]pyridine (13.5 mg, 16%) as a white solid.

4-(1-Methylcyclobutoxy)-N2-(pyridin-2-ylmethyl)pyridine-2,3-diamine was made following a similar procedure to N4-benzyl-2-isopropoxypyridine-3,4-diamine (Synthetic Example 9, intermediate) using 4-(1-methylcyclobutoxy)-3-nitro-N-(pyridin-2-ylmethyl)pyridin-2-amine as the starting material. ¹H NMR (300 MHz, DMSO-d₆): 1.50 (3H, s), 1.6-1.86 (2H, m), 2.06-2.23 (2H, m), 2.30-2.40 (2H, m), 4.15 (2H, s), 4.62 (2H, d), 6.02-6.24 (2H, m), 7.15-7.37 (3H, m), 7.64-7.77 (1H, m), 8.45-8.56 (1H, m). m/z: ES+[M+H]+ 285.

4-(1-Methylcyclobutoxy)-3-nitro-N-(pyridin-2-ylmethyl)pyridin-2-amine was made following a similar procedure to 3-(1-methylcyclobutoxy)-2-nitro-N-(pyridin-2-ylmethyl)aniline (Synthetic Example P1, intermediate) using 2-chloro-4-(1-methylcyclobutoxy)-3-nitropyridine and pyridin-2-ylmethanamine as starting materials. ¹H NMR (300 MHz, DMSO-d₆, 43° C.): 1.57 (3H, s), 1.69-1.83 (2H, m), 2.17-2.4 (4H, m), 4.68 (2H, d), 6.27 (1H, d), 7.21-7.31 (2H, m), 7.68-7.87 (2H, m), 7.98 (1H, d), 8.51 (1H, dt). m/z: ES+[M+H]+ 315.

2-Chloro-4-(1-methylcyclobutoxy)-3-nitropyridine was made following a similar procedure to 9-benzyl-6-(1-methylcyclobutoxy)-9H-purine (Synthetic Example 12, intermediate), using 1-methylcyclobutan-1-ol and 2,4-dichloro-3-nitropyridine as starting materials. ¹H NMR (400 MHz, DMSO-d₆): 1.58 (3H, s), 1.64-1.87 (2H, m), 2.21-2.38 (4H, m), 7.23 (1H, d), 8.40 (1H, d).

[P4] made from a similar procedure as 1-benzyl-2-(2-chloro-4-(2-(piperazin-1-yl)ethoxy)phenyl)-4-(1-methylcyclobutoxy)-1H-benzo[d]imidazole (Synthetic Example P2), using N2-benzyl-4-(1-methylcyclobutoxy)pyridine-2,3-diamine (67.0 mg, 0.24 mmol) and 2-chloro-4-(2-(piperazin-1-yl)ethoxy)benzaldehyde (127 mg, 0.47 mmol).

N2-Benzyl-4-(1-methylcyclobutoxy)pyridine-2,3-diamine was made following a similar procedure to N4-benzyl-2-isopropoxypyridine-3,4-diamine (Synthetic Example 9, intermediate) using N-benzyl-4-(1-methylcyclobutoxy)-3-nitropyridin-2-amine as the starting material. ¹H NMR (400 MHz, DMSO-d₆): 1.49 (3H, s), 1.59-1.82 (2H, m), 1.98-2.23 (2H, m), 2.26-2.43 (2H, m), 4.14 (2H, s), 4.55 (2H, d), 5.98 (1H, t), 6.09 (1H, d), 7.16-7.23 (1H, m), 7.24-7.45 (5H, m). m/z: ES+[M+H]+ 284.

N-Benzyl-4-(1-methylcyclobutoxy)-3-nitropyridin-2-amine was prepared from a similar procedure as 3-(1-methylcyclobutoxy)-2-nitro-N-(pyridin-2-ylmethyl)aniline (Synthetic Example P1 intermediate), using 2-chloro-4-(1-methylcyclobutoxy)-3-nitropyridine (Synthetic Example P3 intermediate) and phenylmethanamine, except that cesium carbonate was used instead of potassium carbonate. ¹H NMR (400 MHz, DMSO-d₆): 1.55 (3H, s), 1.59-1.88 (2H, m), 2.16-2.26 (2H, m), 2.26-2.4 (2H, m), 4.59 (2H, d), 6.24 (1H, d), 7.12-7.25 (1H, m), 7.25-7.33 (4H, m), 7.64 (1H, t), 7.97 (1H, d). m/z: ES+[M+H]+ 314.

The following examples in Table Q were synthesised as stated in the notes at the bottom of Table Q.

TABLE Q

| | | | | LCMS |
|---|---|---|---|---|
| Ex# | Structure | Name | ¹H NMR | [M + H]⁺ |
| Q1 | 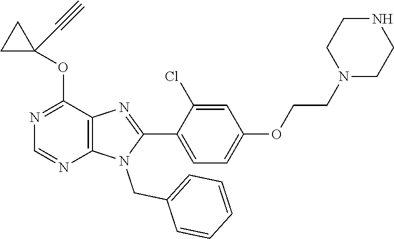 [SMILES: C#CC1(Oc2ncnc3c2nc(-c2ccc(OCCN4CCNCC4)cc2Cl)n3Cc2ccccc2)CC1] | 9-benzyl-8-(2- chloro-4-(2- (piperazin-1- yl)ethoxy)phenyl)- 6-(1- ethynylcyclopro- poxy)-9H-purine | (500 MHz, CDCl₃): 1.42- 1.44 (2H, m), 1.44- 1.46 (2H, m), 2.49 (1H, s), 2.59- 2.66 (4H, m), 2.84 (2H, t), 2.96-3.01 (4H, m), 4.15 (2H, t), 5.35 (2H, s), 6.82 (1H, dd), 6.93 (2H, dd), 7.06 (1H, d), 7.17 (4H, dd), 8.76 (1H, s). 1H not observed. | 529 |
| Q2 | 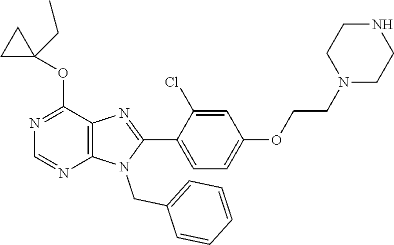 [SMILES: CCC1(Oc2ncnc3c2nc(-c2ccc(OCCN4CCNCC4)cc2Cl)n3Cc2ccccc2)CC1] | 9-benzyl-8-(2- chloro-4-(2- (piperazin-1- yl)ethoxy)phenyl)- 6-(1- ethylcyclopropoxy)- 9H-purine | (500 MHz, CDCl₃): 0.82- 0.85 (2H, m), 1.05 (3H, t), 1.09-1.13 (2H, m), 2.11 (2H, q), 2.56 (4H, s), 2.82 (2H, t), 2.93 (4H, t), 4.15 (2H, t), 5.34 (2H, s), 6.81 (1H, dd), 6.91- 6.96 (2H, m), 7.05 (1H, d), 7.14-7.2 (4H, m), 8.64 (1H, s). 1H not observed. | 533 |
| Q3 | 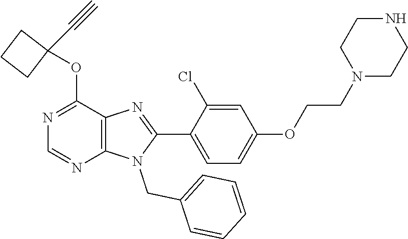 [SMILES: C#CC1(Oc2ncnc3c2nc(-c2ccc(OCCN4CCNCC4)cc2Cl)n3Cc2ccccc2)CCC1] | 9-benzyl-8-(2- chloro-4-(2- (piperazin-1- yl)ethoxy)phenyl)- 6-(1- ethynylcyclbu- toxy)-9H-purine | (500 MHz, CDCl₃): 1.97- 2.01 (1H, m), 2.04- 2.09 (1H, m), 2.61 (1H, d), 2.69 (4H, s), 2.71-2.76 (2H, m), 2.8-2.87 (4H, m), 3.03 (4H, s), 4.15 (2H, t), 5.34 (2H, s), 6.81 (1H, d), 6.92 (2H, d), 7.05 (1H, s), 7.15-7.19 (4H, m), 8.64 (1H, s). 1H not observed. | 543 |
| Q4 | 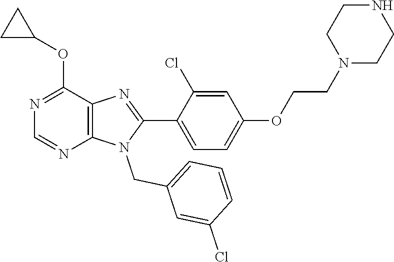 [SMILES: Clc1cccc(Cn2c(-c3ccc(OCCN4CCNCC4)cc3Cl)nc3c(OC4CC4)ncnc32)c1] | 8-(2-chloro-4-(2- (piperazin-1- yl)ethoxy)phenyl)- 9-(3- chlorobenzyl)-6- cyclopropoxy- 9H-purine | (500 MHz, CDCl₃): 0.86- 0.92 (2H, m), 0.96- 1.02 (2H, m), 2.61 (4H, s), 2.84 (2H, t), 2.94-3 (4H, m), 4.16 (2H, t), 4.61-4.71 (1H, m), 5.31 (2H, s), 6.81-6.87 (3H, m), 7.06 (1H, d), 7.11 (1H, t), 7.13-7.18 (2H, m), 8.66 (1H, s). 1H not observed. | 539 |
| Q5 | 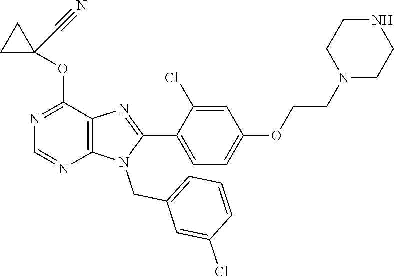 [SMILES: N#CC1(Oc2ncnc3c2nc(-c2ccc(OCCN4CCNCC4)cc2Cl)n3Cc2cccc(Cl)c2)CC1] | 1-((8-(2-chloro- 4-(2-(piperazin- 1- yl)ethoxy)phenyl)- 9-(3- chlorobenzyl)- 9H-purin-6- yl)oxy)cyclopro- pane-1-carbonitrile | (500 MHz, CDCl₃): 1.57 (2H, t), 1.69 (2H, t), 2.65 (4H, s), 2.85 (2H, t), 3.01 (4H, s), 4.16 (2H, t), 5.33 (2H, s), 6.81-6.87 (3H, m), 7.08 (1H, d), 7.09-7.19 (3H, m), 8.75 (1H, s). 1H not observed. | 564 |

[Q1] was made from a similar procedure as 2-(2-chloro-4-(2-(piperazin-1-yl)ethoxy)phenyl)-1-((4-chloropyridin-2-yl)methyl)-4-(1-methylcyclopropoxy)-1H-imidazo[4,5-c]pyridine (Synthetic Example 16), using tert-butyl 4-(2-(4-(9-benzyl-6-(1-ethynylcyclopropoxy)-9H-purin-8-yl)-3-chlorophenoxy)ethyl)piperazine-1-carboxylate as starting material and 1,4-dioxane as solvent.

Tert-butyl 4-(2-(4-(9-benzyl-6-(1-ethynylcyclopropoxy)-9H-purin-8-yl)-3-chlorophenoxy)ethyl)piperazine-1-carboxylate was made from a similar procedure as tert-butyl 4-(2-(4-(9-benzyl-6-(1-methylcyclopropoxy)-9H-purin-8-yl)-3-chlorophenoxy)ethyl)piperazine-1-carboxylate (Synthetic Example 6, intermediate), using tert-butyl 4-(2-(4-(9-benzyl-6-chloro-9H-purin-8-yl)-3-chlorophenoxy)ethyl)piperazine-1-carboxylate (Synthetic Example 6, intermediate) and 1-ethynylcyclopropan-1-ol as starting materials. The crude product was used in next reaction without further purification. m/z: ES+[M+H]+ 629.

[Q2] was made from a similar procedure as 2-(2-chloro-4-(2-(piperazin-1-yl)ethoxy)phenyl)-1-((4-chloropyridin-2-yl)methyl)-4-(1-methylcyclopropoxy)-1H-imidazo[4,5-c]pyridine (Synthetic Example 16), using tert-butyl 4-(2-(4-(9-benzyl-6-(1-ethylcyclopropoxy)-9H-purin-8-yl)-3-chlorophenoxy)ethyl)piperazine-1-carboxylate as starting material and 1,4-dioxane as solvent.

Tert-butyl 4-(2-(4-(9-benzyl-6-(1-ethylcyclopropoxy)-9H-purin-8-yl)-3-chlorophenoxy)ethyl)piperazine-1-carboxylate was made from a similar procedure as tert-butyl 4-(2-(4-(9-benzyl-6-(1-methylcyclopropoxy)-9H-purin-8-yl)-3-chlorophenoxy)ethyl)piperazine-1-carboxylate (Synthetic Example 6, intermediate), using tert-butyl 4-(2-(4-(9-benzyl-6-chloro-9H-purin-8-yl)-3-chlorophenoxy)ethyl)piperazine-1-carboxylate (Synthetic Example 6, intermediate) and 1-ethylcyclopropan-1-ol as starting materials. The crude product was used in next reaction without further purification. m/z: ES+[M+H]+ 633.

[Q3] was made from a similar procedure as tert-butyl 4-(2-(4-(9-benzyl-6-(1-methylcyclopropoxy)-9H-purin-8-yl)-3-chlorophenoxy)ethyl)piperazine-1-carboxylate (Synthetic Example 6, intermediate), using 4-(2-(4-(9-benzyl-6-chloro-9H-purin-8-yl)-3-chlorophenoxy)ethyl)piperazin-1-ium chloride and 1-ethynylcyclobutan-1-ol as starting materials, and DMF as solvent.

4-(2-(4-(9-Benzyl-6-chloro-9H-purin-8-yl)-3-chlorophenoxy)ethyl)piperazin-1-ium chloride was made from a similar procedure as 2-(2-chloro-4-(2-(piperazin-1-yl)ethoxy)phenyl)-1-((4-chloropyridin-2-yl)methyl)-4-(1-methylcyclopropoxy)-1H-imidazo[4,5-c]pyridine (Synthetic Example 16), using tert-butyl 4-(2-(4-(9-benzyl-6-chloro-9H-purin-8-yl)-3-chlorophenoxy)ethyl)piperazine-1-carboxylate (Synthetic Example 6, intermediate) as starting material and 1,4-dioxane as solvent. The crude product was used in next reaction without further purification. m/z: ES+[M+H]+ 483.

[Q4] was made from a similar procedure as 2-(2-chloro-4-(2-(piperazin-1-yl)ethoxy)phenyl)-1-((4-chloropyridin-2-yl)methyl)-4-(1-methylcyclopropoxy)-1H-imidazo[4,5-c]pyridine (Synthetic Example 16), using tert-butyl 4-(2-(3-chloro-4-(9-(3-chlorobenzyl)-6-cyclopropoxy-9H-purin-8-yl)phenoxy)ethyl)piperazine-1-carboxylate as starting material and 1,4-dioxane as solvent.

Tert-butyl 4-(2-(3-chloro-4-(9-(3-chlorobenzyl)-6-cyclopropoxy-9H-purin-8-yl)phenoxy)ethyl)piperazine-1-carboxylate was made from a similar procedure as tert-butyl 4-(2-(4-(9-benzyl-6-(1-methylcyclopropoxy)-9H-purin-8-yl)-3-chlorophenoxy)ethyl)piperazine-1-carboxylate (Synthetic Example 6, intermediate), using tert-butyl 4-(2-(3-chloro-4-(6-chloro-9-(3-chlorobenzyl)-9H-purin-8-yl)phenoxy)ethyl)piperazine-1-carboxylate and cyclopropanol as starting materials. The crude product was used in next reaction without further purification. m/z: ES+[M+H]+ 639.

Tert-butyl 4-(2-(3-chloro-4-(6-chloro-9-(3-chlorobenzyl)-9H-purin-8-yl)phenoxy)ethyl)piperazine-1-carboxylate was made from a similar procedure as tert-butyl 4-(2-(3-chloro-4-(6-chloro-9-((4-chloropyridin-2-yl)methyl)-9H-purin-8-yl)phenoxy)ethyl)piperazine-1-carboxylate (Synthetic Example 10, intermediate), using tert-butyl 4-(2-(3-chloro-4-(6-chloro-9H-purin-8-yl)phenoxy)ethyl)piperazine-1-carboxylate (Synthetic Example 10, intermediate) and 1-chloro-3-(chloromethyl)benzene as starting materials. ¹H NMR (500 MHz, CDCl₃): 1.47 (9H, s), 2.52-2.57 (4H, m), 2.86 (2H, t), 3.47 (4H, s), 4.18 (2H, t), 5.35 (2H, s), 6.82-6.9 (3H, m), 7.09-7.14 (2H, m), 7.19 (2H, d), 8.81 (1H, s). m/z: ES+[M+H]+ 617.

[Q5] was made from a similar procedure as 2-(2-chloro-4-(2-(piperazin-1-yl)ethoxy)phenyl)-1-((4-chloropyridin-2-yl)methyl)-4-(1-methylcyclopropoxy)-1H-imidazo[4,5-c]pyridine (Synthetic Example 16), using tert-butyl 4-(2-(3-chloro-4-(9-(3-chlorobenzyl)-6-(1-cyanocyclopropoxy)-9H-purin-8-yl)phenoxy)ethyl)piperazine-1-carboxylate as starting material and acetonitrile as solvent, except that the reaction mixture was kept at 0° C.

Tert-butyl 4-(2-(3-chloro-4-(9-(3-chlorobenzyl)-6-(1-cyanocyclopropoxy)-9H-purin-8-yl)phenoxy)ethyl)piperazine-1-carboxylate was made from a similar procedure as tert-butyl 4-(2-(4-(9-benzyl-6-(1-methylcyclopropoxy)-9H-purin-8-yl)-3-chlorophenoxy)ethyl)piperazine-1-carboxylate (Synthetic Example 6, intermediate), using tert-butyl 4-(2-(3-chloro-4-(6-chloro-9-(3-chlorobenzyl)-9H-purin-8-yl)phenoxy)ethyl)piperazine-1-carboxylate (Synthetic Example Q4, intermediate) and 1-hydroxycyclopropane-1-carbonitrile as starting materials. The crude product was used in next reaction without further purification. m/z: ES+[M+H]+ 664.

The following examples in Table R were synthesised as stated in the notes at the bottom of Table R.

TABLE R

| | | | | LCMS |
|---|---|---|---|---|
| Ex# | Structure | Name | ¹H NMR | [M + H]⁺ |
| R1 | 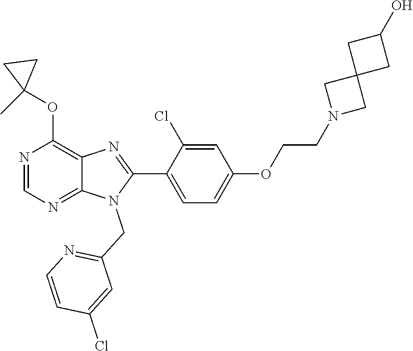 | 2-(2-(3-chloro-4- (9-((4- chloropyridin-2- yl)methyl)-6-(1- methylcyclopro- poxy)-9H-purin-8- yl)phenoxy)ethyl)- 2-azaspiro[3.3] heptan-6-ol | (400 MHz, DMSO-d₆): 0.85 (2H, t), 1.02 (2H, t), 1.73 (3H, s), 1.8-1.98 (2H, m), 2.21- 2.4 (2H, m), 2. 67 (2H, t), 3.12 (4H, d), 3.78-4.05 (3H, m), 4.94 (1H, d), 5.40 (2H, s), 6. 93 (1H, dd), 7.15 (1H, d), 7.26 (1H, d), 7.3-7. 44 (2H, m), 8.30 (1H, d), 8.57 (1H, s). | 581 |
| R2 | 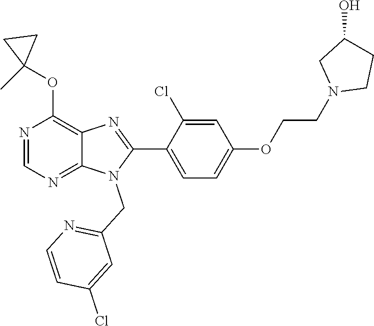 | (R)-1-(2-(3- chloro-4-(9-((4- chloropyridin-2- yl)methyl)-6-(1- methylcyclopro- poxy)-9H-purin-8- yl)phenoxy)ethyl) pyrrolidin-3-ol | (300 MHz, DMSO-d₆): 0.84 (2H, t), 0.99 (2H, t), 1.46-1. 58 (1H, m), 1.73 (3H, s), 1.88-2.04 (1H, m), 2.37-2.42 (1H, m), 2.59-2.69 (1H, m), 2.73-2.82 (3H, m), 4.10-4.38 (3H, m), 4.66-4.72 (1H, m), 5.40 (2H, s), 6.96-7.00 (1H, m), 7.11-7.21 (1H, m), 7.22-7.29 (1H, m), 7.34-7.41 (2H, m), 8.31 (1H, d), 8.57 (1H, s). One proton not observed. | 555 |
| R3 | 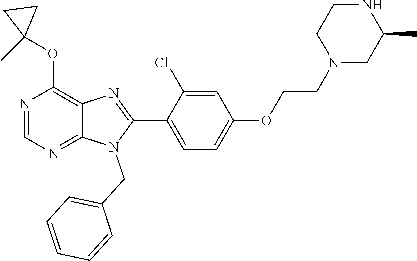 | (S)-9-benzyl-8- (2-chloro-4-(2- (3- methylpiperazin- 1- yl)ethoxy)phenyl)- 6-(1- methylcyclopro- poxy)-9H-purine | (400 MHz, DMSO-d₆): 0.84 (2H, t), 0. 91 (3H, d), 1.03 (2H, t), 1.63 (1H, t), 1.73 (3H, s), 1.9-2.03 (1H, m), 2.6-2.7 (4H, m), 2.72-2.83 (3H, m), 4.18 (2H, t), 5.29 (2H, s), 6.82-6.96 (2H, m), 7-7.07 (1H, m), 7.16- 7.23 (3H, m), 7.25 (1H, d), 7.40 (1H, d), 8.61 (1H, s). One proton not observed. | 533 |
| R4 | 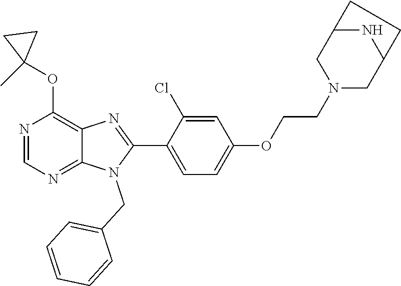 | 8-(4-(2-(3,8- diazabicyclo[3.2.1] octan-3- yl)ethoxy)-2- chlorophenyl)-9- benzyl-6-(1- methylcyclopro- poxy)-9H-purine | (400 MHz, DMSO-d₆): 0.84 (2H, t), 1.02 (2H, t), 1.48-1.56 (2H, m), 1.63-1.71 (2H, m), 1.73 (3H, s), 2.22 (2H, d), 2.57-2.7 (4H, m), 3.19-3.35 (2H, m), 4.16 (2H, t), 5.29 (2H, s), 6.85-6.94 (2H, m), 6.99-7.06 (1H, m), 7.14-7.23 (3H, m), 7.25 (1H, d), 7.38 (1H, d), 8.61 (1H, s). One proton not observed. | 545 |
| R5 | 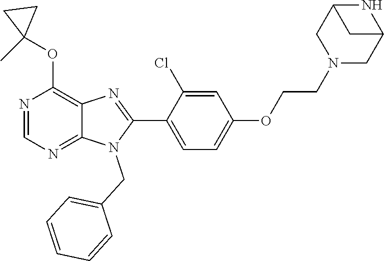 | 3-(2-(4-(9- benzyl-6-(1- methylcyclopro- poxy)-9H-purin-8- yl)-3- chlorophenoxy)eth- yl)-3,6- diazabicyclo[3.1.1] heptane | (400 MHz, DMSO-d₆): 0.84 (2H, t), 1.01 (2H, t), 1.73 (3H, s), 1.74-1.76 (1H, m), 2.17- 2.26 (1H, m), 2.74 (2H, d), 2.94 (2H, t), 3.10 (2H, d), 3.43 (2H, d), 4.23 (2H, t), 5.29 (2H, s), 6.87-6.95 (2H, m), 7.02-7.08 (1H, m), 7.17-7.23 (3H, m), 7.27 (1H, d), 7.41 (1H, d), 8.61 (1H, s). One proton not observed. | 531 |
| R6 | 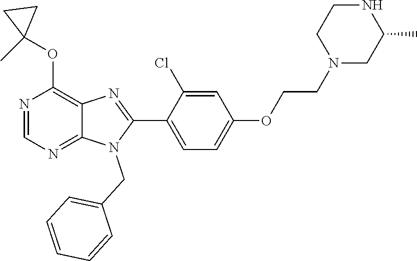 | (R)-9-benzyl-8- (2-chloro-4-(2- (3- methylpiperazin- 1- yl)ethoxy)phenyl)- 6-(1- methylcyclopro- poxy)-9H-purine | (400 MHz, DMSO-d₆): 0.84 (2H, t), 0.91 (3H, d), 1.03 (2H, t), 1.63 (1H, t), 1.73 (3H, s), 1.9-2.03 (1H, m), 2.6-2.7 (4H, m), 2.72-2.83 (3H, m), 3.51 (1H, s), 4.18 (2H, t), 5.29 (2H, s), 6.82- 6.96 (2H, m), 7-7.07 (1H, m), 7.16-7.23 (3H, m), 7.25 (1H, d), 7.40 (1H, d), 8.61 (1H, s). | 533 |
| R7 | 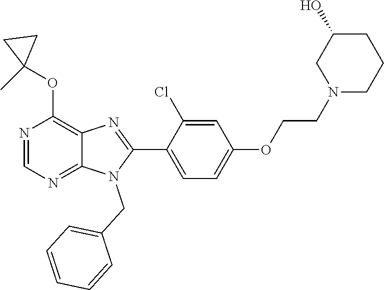 | (R)-1-(2-(4-(9- benzyl-6-(1- methylcyclopro- poxy)-9H-purin-8- yl)-3- chlorophenoxy)eth- yl)piperidin-3-ol | (400 MHz, CDCl₃): 0.85 (2H, t), 1.16 (2H, t), 1.6-1.74 (3H, m), 1.81 (3H, s), 1.91-2.02 (1H, m), 2.66-3.07 (6H, m), 3.95-4 (1H, m), 4.25 (2H, t), 5.35 (2H, s), 6.79-6.87 (1H, m), 6.92-6.97 (2H, m), 7.07 (1H, d), 7.14-7. 23 (4H, m), 8.68 (1H, s). One proton not observed. | 534 |
| R8 | 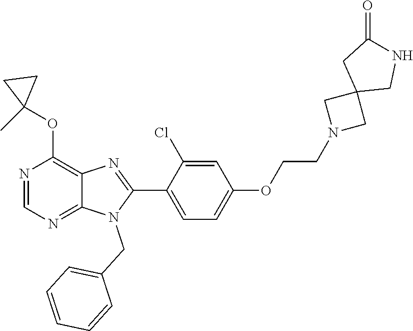 | 2-(2-(4-(9- benzyl-6-(1- methylcyclopro- poxy)-9H-purin-8- yl)-3- chlorophenoxy)eth- yl)-2,6- diazaspiro[3.4] octan-7-one | (400 MHz, DMSO-d₆): 0.84 (2H, t), 1.01 (2H, t), 1.73 (3H, s), 2.34 (2H, s), 2.75 (2H, t), 3.21 (4H, t), 3.37 (2H, s), 4.05 (2H, t), 5.29 (2H, s), 6.87- 6.93 (2H, m), 7.01 (1H, dd), 7.18-7.23 (4H, m), 7.40 (1H, d), 7.54 (1H, s), 8.61 (1H, s). | 559 |
| R9 | 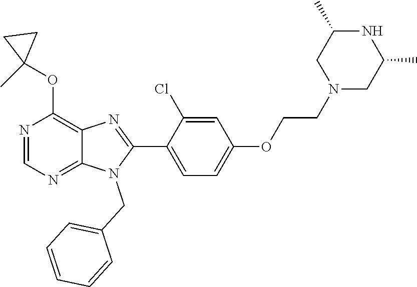 | (cis)-9-benzyl-8- (2-chloro-4-(2- (3,5- dimethylpiperazin- 1- yl)ethoxy)phenyl)- 6-(1- methylcyclopro- poxy)-9H-purine | (400 MHz, DMSO-d₆): 0.83 (2H, t), 0.93-1.06 (8H, m), 1.73 (3H, s), 2.15-2.28 (2H, m), 2.43-2.49 (2H, m), 2.66-2.74 (2H, m), 2.97 (2H, t), 3. 54 (1H, s), 4.09 (2H, t), 5.29 (2H, s), 6.87-6.93 (2H, m), 6.99-7.04 (1H, m), 7.17-7.22 (3H, m), 7.24 (1H, d), 7.41 (1H, d), 8.61 (1H, s). | 547 |
| | Relative | | | |
| | stereochemistry | | | |
| R10 | 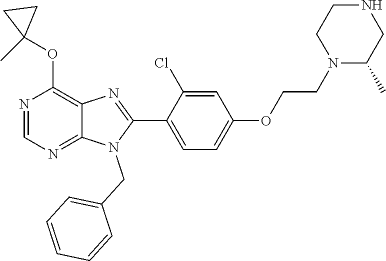 | (S)-9-benzyl-8- (2-chloro-4-(2- (2- methylpiperazin- 1- yl)ethoxy)phenyl)- 6-(1- methylcyclopro- poxy)-9H-purine | (400 MHz, DMSO-d₆): 0.83 (2H, t), 0.96 (3H, d), 1.01 (2H, t), 1.73 (3H, s), 2.21-2.38 (3H, m), 2.55-2.65 (2H, m), 2.67-2.81 (3H, m), 2.96-3.06 (1H, m), 4.15 (2H, t), 5.29 (2H, s), 6.87- 6.93 (2H, m), 7-7.05 (1H, m), 7.17-7.22 (3H, m), 7.25 (1H, d), 7.40 (1H, d), 8.61 (1H, s). One proton not observed. | 533 |
| R11 | 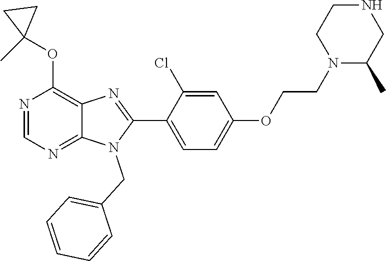 | (R)-9-benzyl-8- (2-chloro-4-(2- (2- methylpiperazin- 1- yl)ethoxy)phenyl)- 6-(1- methylcyclopro- poxy)-9H-purine | (300 MHz, DMSO-d₆): 0.83 (2H, t), 0.96 (3H, d), 1.01 (2H, t), 1.73 (3H, s), 2.21-2.38 (3H, m), 2.55-2.65 (2H, m), 2.68-2.8 (3H, m), 2.98-3.06 (1H, m), 4.15 (2H, t), 5.29 (2H, s), 6.85-6.92 (2H, m), 7-7.05 (1H, m), 7.16-7.22 (3H, m), 7.25 (1H, d), 7.40 (1H, d), 8.61 (1H, s). One proton not observed. | 533 |
| R12 | 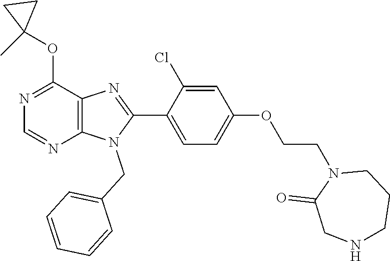 | 1-(2-(4-(9- benzyl-6-(1- methylcyclopro- poxy)-9H-purin-8- yl)-3- chlorophenoxy)eth- yl)-1,4- diazepan-2-one | (400 MHz, DMSO- d₆): 0.85 (2H, t), 1. 01 (2H, t), 1.52-1.65 (2H, m), 1.73 (3H, s), 2.79-2.89 (2H, m), 3.48- 3.6 (2H, m), 3.63- 3.75 (2H, m), 3.67 (2H, t), 4.15 (2H, t), 5.29 (2H, s), 6.85- 6.94 (2H, m), 6.99-7.07 (1H, m), 7.15-7.22 (3H, m), 7.25 (1H, d), 7.41 (1H, d), 8. 61 (1H, s). One proton not observed. | 547 |
| R13 | 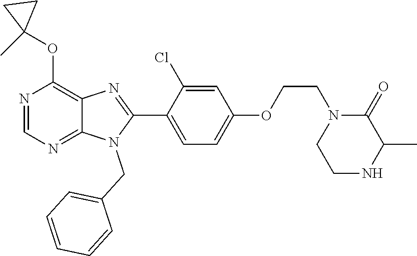 Isomer 1 | 1-(2-(4-(9- benzyl-6-(1- methylcyclopro- poxy)-9H-purin-8- yl)-3- chlorophenoxy)eth- yl)-3- methylpiperazin- 2-one (Isomer 1) | (400 MHz, DMSO-d₆): 0.84 (2H, t), 1. 03 (2H, t), 1.18 (3H, d), 1.73 (3H, s), 2.66 (1H, s), 2.74-2.85 (1H, m), 2.93-3.01 (1H, m), 3.21-3.3 (1H, m), 3.33-3.36 (1H, m), 3.43-3.51 (1H, m), 3.59-3.68 (2H, m), 4. 22 (2H, t), 5.29 (2H, s), 6.84-6.96 (2H, m), 7.02-7.07 (1H, m), 7.16-7.23 (3H, m), 7.26 (1H, d), 7.41 (1H, d), 8.61 (1H, s). | 547 |
| R14 | 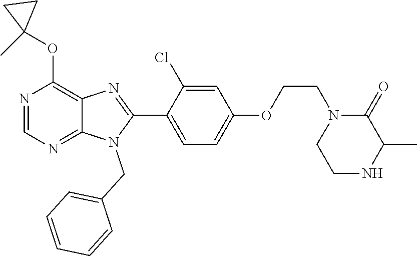 Isomer 2 | 1-(2-(4-(9- benzyl-6-(1- methylcyclopro- poxy)-9H-purin-8- yl)-3- chlorophenoxy)eth- yl)-3- methylpiperazin- 2-one (Isomer 2) | (400 MHz, DMSO-d₆): 0.84 (2H, t), 1.02 (2H, t), 1.18 (3H, d), 1.73 (3H, s), 2.63 (1H, s), 2.75-2.84 (1H, m), 2.93-3.01 (1H, m), 3.22-3.3 (1H, m), 3.33-3.36 (1H, m), 3.42-3.51 (1H, m), 3.57-3.69 (2H, m), 4.22 (2H, t), 5.29 (2H, s), 6.87-6.94 (2H, m), 7.02-7.08 (1H, m), 7.16-7.23 (3H, m), 7.26 (1H, d), 7.41 (1H, d), 8.61 (1H, s). | 547 |
| R15 | 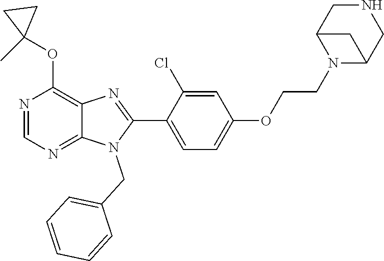 | 6-(2-(4-(9- benzyl-6-(1- methylcyclopro- poxy)-9H-purin-8- yl)-3- chlorophenoxy)eth- yl)-3,6- diazabicyclo[3.1.1] heptane | (400 MHz, DMSO-d₆): 0.83 (2H, t), 1.02 (2H, t), 1.72-1.74 (4H, m), 2.33-2.41 (1H, m), 2.65-2.75 (1H, m), 2.84-2.9 (2H, m), 3.24-3.3 (2H, m), 3.38-3.46 (2H, m), 3.52-3.65 (2H, m), 4.10 (2H, t), 5.29 (2H, s), 6.88-6.93 (2H, m), 6.98-7.03 (1H, m), 7.17-7.22 (4H, m), 7.40 (1H, d), 8 61 (1H, s). | 531 |
| R16 | 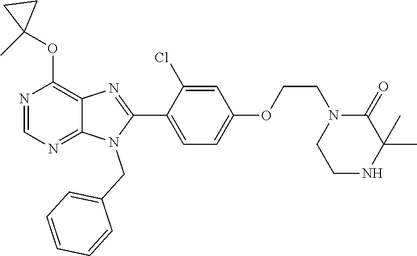 | 1-(2-(4-(9- benzyl-6-(1- methylcyclopro- poxy)-9H-purin-8- yl)-3- chlorophenoxy)eth- yl)-3,3- dimethylpiperazin- 2-one | (400 MHz, DMSO-d₆): 0.83 (2H, t), 1.02 (2H, t), 1.18 (6H, s), 1.73 (3H, s), 2.38 (1H, s), 2.88 (2H, t), 3.40 (2H, t), 3. 62 (2H, t), 4.21 (2H, t), 5.29 (2H, s), 6.87-6.94 (2H, m), 7.02-7.07 (1H, m), 7.17-7.22 (3H, m), 7.26 (1H, d), 7.41 (1H, d), 8.62 (1H, s). | 561 |
| R17 | 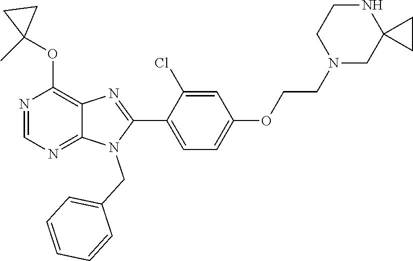 | 8-(4-(2-(4,7- diazaspiro[2.5] octan-7- yl)ethoxy)-2- chlorophenyl)-9- benzyl-6-(1- methylcyclopro- poxy)-9H-purine | (400 MHz DMSO-d₆): 0.28-0.45 (4H, m), 0.85 (2H, t), 1.01 (2H, t), 1.73 (3H, s), 2. 01 (1H, s), 2.28 (2H, s), 2.37-2.46 (2H, m), 2.66 (2H, t), 2.73 (2H, t), 4.17 (2H, t), 5.28 (2H, s), 6.85-6.93 (2H, m), 7.03 (1H, dd), 7.16-7.22 (3H, m), 7.25 (1H, d), 7.40 (1H, d), 8.61 (1H, s). | 545 |
| R18 | 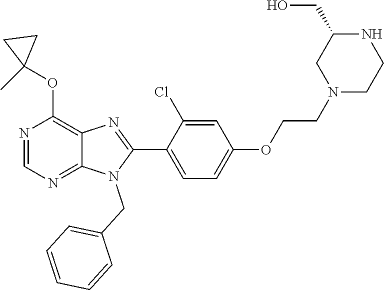 | (R)-(4-(2-(4-(9- benzyl-6-(1- methylcyclopro- poxy)-9H-purin-8- yl)-3- chlorophenoxy)eth- yl)piperazin-2- yl)methanol | (400 MHz, DMSO-d₆): 0.85 (2H, t), 1.01 (2H, t), 1.66-1.75 (4H, m), 1.96-2.07 (2H, m), 2.6-2.71 (4H, m), 2.72-2.79 (1H, m), 2.8-2.86 (2H, m), 3.27 (2H, t), 4.18 (2H, t), 4.51 (1H, t), 5.29 (2H, s), 6.88-6.92 (2H, m), 7.01- 7.05 (1H, m), 7.16- 7.28 (4H, m), 7.40 (1H, d), 8.61 (1H, s). | 549 |
| R19 | 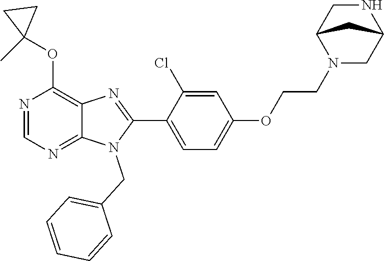 | 8-(4-(2-((1S,4S)- 2,5- diazabicyclo[2.2.1] heptan-2- yl)ethoxy)-2- chlorophenyl)-9- benzyl-6-(1- methylcyclopro- poxy)-9H-purine | (400 MHz, DMSO-d₆): 0.85 (2H, t), 1.01 (2H, t), 1.40 (1H, d), 1.60 (1H, d), 1.73 (3H, s), 2.32-2.43 (1H, m), 2.57-2.68 (1H, m), 2.78-3.02 (4H, m), 3.34-3.52 (3H, m), 4.11 (2H, t), 5.29 (2H, s), 6.74-6.98 (2H, m), 6.99-7.05 (1H, m), 7.11-7.29 (4H, m), 7.40 (1H, d), 8.61 (1H, s). | 531 |
| R20 | 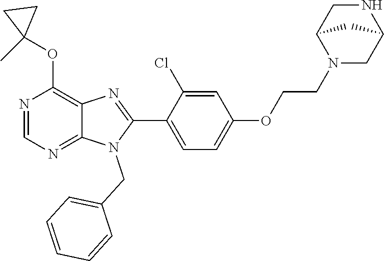 | 8-(4-(2-((1R,4R)- 2,5- diazabicyclo[2.2.1] heptan-2- yl)ethoxy)-2- chlorophenyl)-9- benzyl-6-(1- methylcyclopro- poxy)-9H-purine | (400 MHz, DMSO-d₆): 0.84 (2H, t), 1.01 (2H, t), 1.41 (1H, d), 1.60 (1H, d), 1.73 (3H, s), 2.33-2.41 (1H, m), 2.6-2.69 (1H, m), 2.78-2.97 (4H, m), 3.28-3.4 (2H, m), 3.54 (1H, s), 4.10 (2H, t), 5.29 (2H, s), 6.85-6.96 (2H, m), 6.98-7.06 (1H, m), 7.16-7.3 (4H, m), 7.40 (1H, d), 8.61 (1H, s). | 531 |
| R21 | 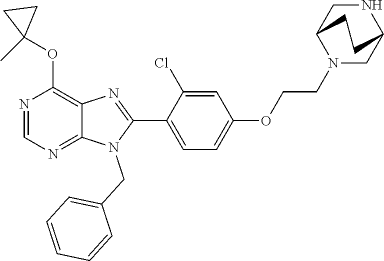 | (1S,4S)-2-(2-(4- (9-benzyl-6-(1- methylcyclopro- poxy)-9H-purin-8- yl)-3- chlorophenoxy)eth- yl)-2,5- diazabicyclo[2.2.2] octane | (400 MHz, DMSO-d₆): 0.84 (2H, t), 1. 01 (2H, t), 1.48-1.65 (2H, m), 1.63-1.81 (4H, m), 1.82-2.00 (1H, m), 2.57-2.73 (2H, m), 2.75-2.81 (1H, m), 2.84-2.96 (4H, m), 3.20 (1H, d), 3.86 (1H, s), 4.13 (2H, t), 5.29 (2H, s), 6.81-6.93 (2H, m), 6.98- 7.11 (1H, m), 7.15- 7.29 (4H, m), 7.40 (1H, d), 8.61 (1H, s). | 545 |
| R22 | 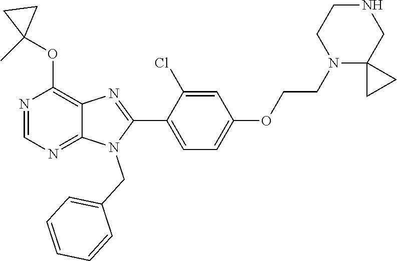 | 8-(4-(2-(4,7- diazaspiro[2.5] octan-4- yl)ethoxy)-2- chlorophenyl)-9- benzyl-6-(1- methylcyclopro- poxy)-9H-purine | (400 MHz, DMSO-d₆): 0.39 (2H, t), 0.51 (2H, t), 0.85 (2H, t), 1.01 (2H, t), 1.73 (3H, s), 2.69 (2H, s), 2.83 (2H, t), 3.04 (2H, t), 3.22- 3.44 (3H, m), 4.03 (2H, t), 5.28 (2H, s), 6.87-6.94 (2H, m), 6.97-7.02 (1H, m), 7.17- 7.22 (4H, m), 7.38 (1H, d), 8.62 (1H, s). | 545 |
| R23 | 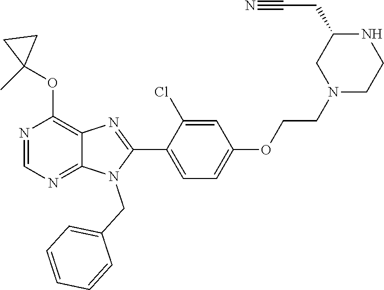 | (S)-2-(4-(2-(4-(9- benzyl-6-(1- methylcyclopro- poxy)-9H-purin-8- yl)-3- chlorophenoxy)eth- yl)piperazin-2- yl)acetonitrile | (400 MHz, DMSO-d₆): 0.84 (2H, t), 1.03 (2H, t), 1.72 (3H, s), 1.85-1.96 (1H, m), 2.01- 2.12 (1H, m), 2.2- 2.4 (1H, m), 2.55- 2.6 (2H, m), 2.62-2.77 (4H, m), 2.79-2.91 (3H, m), 4.18 (2H, t), 5.29 (2H, s), 6.86- 6.96 (2H, m), 7.00-7.08 (1H, m), 7.16-7.23 (3H, m), 7.25 (1H, d), 7.40 (1H, d), 8.61 (1H, s). | 558 |

[R1] was made by alkylation of 3-chloro-4-(9-((4-chloropyridin-2-yl)methyl)-6-(1-methylcyclopropoxy)-9H-purin-8-yl)phenol (Synthetic Example 17 intermediate) with 2-(2-chloroethyl)-2-azaspiro[3.3]heptan-6-ol following a similar procedure as Synthetic Example 17, except that the reaction was heated at 80° C.

2-(2-Chloroethyl)-2-azaspiro[3.3]heptan-6-ol was synthesised as follows:

1-Bromo-2-chloroethane (456 mg, 3.18 mmol) was added to 2-azaspiro[3.3]heptan-6-ol (300 mg, 2.65 mmol) and K₂CO₃ (733 mg, 5.30 mmol) in MeCN (5 mL). The resulting mixture was stirred at 80° C. for 2 hours. The solvent was removed under reduced pressure. The crude product was purified by flash silica chromatography, elution gradient 0 to 30% EtOAc in petroleum ether. Pure fractions were evaporated to dryness to afford 2-(2-chloroethyl)-2-azaspiro[3.3]heptan-6-ol (200 mg, 43%) as a yellow solid. ¹H NMR (300 MHz, CDCl₃): 2.00-2.06 (2H, m), 2.49-2.56 (2H, m), 2.73-2.77 (2H, m), 3.29 (4H, d), 3.43-3.47 (2H, m), 4.15-4.23 (1H, m). One proton not observed. m/z: ES+[M+H]+ 176.

[R2] was synthesised as follows:

(R)-Pyrrolidin-3-ol (34.3 mg, 0.39 mmol) was added to 8-(4-(2-bromoethoxy)-2-chlorophenyl)-9-((4-chloropyridin-2-yl)methyl)-6-(1-methylcyclopropoxy)-9H-purine (180 mg, 0.33 mmol), sodium iodide (246 mg, 1.64 mmol) and DIEA (0.172 mL, 0.98 mmol) in DMF (5 mL). The resulting mixture was stirred at 35° C. for 12 hours. The resulting mixture was evaporated to dryness to afford crude product. The crude product was purified by preparative HPLC, XBridge Prep OBD C18 column, 30*150 mm, 5 μm; using decreasingly polar mixtures of water (containing 10 mmol/L NH₄HCO₃ and 0.1% aqueous ammonia) and MeCN as eluents. Fractions containing the desired compound were evaporated to dryness to afford (R)-1-(2-(3-chloro-4-(9-((4-chloropyridin-2-yl)methyl)-6-(1-methylcyclopropoxy)-9H-purin-8-yl)phenoxy)ethyl)pyrrolidin-3-ol (47 mg, 26%) as a white solid.

8-(4-(2-Bromoethoxy)-2-chlorophenyl)-9-((4-chloropyridin-2-yl)methyl)-6-(1-methylcyclopropoxy)-9H-purine was synthesised as follows:

3-Chloro-4-(9-((4-chloropyridin-2-yl)methyl)-6-(1-methylcyclopropoxy)-9H-purin-8-yl)phenol (800 mg, 1.81 mmol, Synthetic Example 17 intermediate) was added to 1,2-dibromoethane (3.40 g, 18.1 mmol) and K₂CO₃ (875 mg, 6.33 mmol) in MeCN (30 mL) at 25° C. The resulting mixture was stirred at 60° C. for 16 hours. The reaction mixture was poured into water (300 mL) and extracted with EtOAc (3×300 mL). The organic layer was dried over Na₂SO₄, filtered and evaporated to afford crude product. The crude product was purified by flash silica chromatography, elution gradient 50 to 70% EtOAc in petroleum ether. Pure fractions were evaporated to dryness to afford 8-(4-(2-bromoethoxy)-2-chlorophenyl)-9-((4-chloropyridin-2-yl)methyl)-6-(1-methylcyclopropoxy)-9H-purine (700 mg, 70%) as a green oil. ¹H NMR (300 MHz, DMSO-d₆): 0.86 (2H, t), 1.03 (2H, t), 1.74 (3H, s), 3.83 (2H, t), 4.43 (2H, t), 5.41 (2H, s), 7.02 (1H, dd), 7.24 (1H, d), 7.28 (1H, d), 7.37-7.42 (2H, m), 8.31 (1H, d), 8.58 (1H, s). m/z: ES+[M+H]+ 548.

[R3] was made by an N-alkylation reaction of 9-benzyl-8-(4-(2-bromoethoxy)-2-chlorophenyl)-6-(1-methylcyclopropoxy)-9H-purine with tert-butyl (S)-2-methylpiperazine-1-carboxylate using a similar procedure as Synthetic Example R2, except that the reaction was run in DMF at 35° C. to give tert-butyl (S)-4-(2-(4-(9-benzyl-6-(1-methylcyclopropoxy)-9H-purin-8-yl)-3-chlorophenoxy)ethyl)-2-methylpiperazine-1-carboxylate. ¹H NMR (300 MHz, DMSO-d₆): 0.83 (2H, t), 1.00 (2H, t), 1.11 (3H, d), 1.38 (9H, s), 1.71 (3H, s), 1.92-2.04 (1H, m), 2.04-2.22 (1H, m), 2.59-2.8 (3H, m), 2.8-3.05 (2H, m), 3.54-3.72 (1H, m), 3.93-4.12 (1H, m), 4.19 (2H, t), 5.27 (2H, s), 6.83-6.96 (2H, m), 7.03 (1H, dd), 7.13-7.21 (3H, m), 7.25 (1H, d), 7.40 (1H, d), 8.60 (1H, s). m/z: ES+[M+H]+ 633.

BOC deprotection of tert-butyl (S)-4-(2-(4-(9-benzyl-6-(1-methylcyclopropoxy)-9H-purin-8-yl)-3-chlorophenoxy)ethyl)-2-methylpiperazine-1-carboxylate using TFA in DCM provided Synthetic Example R3.

9-Benzyl-8-(4-(2-bromoethoxy)-2-chlorophenyl)-6-(1-methylcyclopropoxy)-9H-purine was made through a Williamson ether synthesis reaction from 4-(9-benzyl-6-(1-methylcyclopropoxy)-9H-purin-8-yl)-3-chlorophenol (Synthetic Example 5 intermediate) and 1,2-dibromoethane as follows:

1,2-Dibromoethane (11.5 g, 61.4 mmol) was added to 4-(9-benzyl-6-(1-methylcyclopropoxy)-9H-purin-8-yl)-3-chlorophenol (5.0 g, 12.3 mmol) and K₂CO₃ (2.55 g, 18.4 mmol) in MeCN (20 mL) at 25° C. The resulting mixture was stirred at 80° C. for 4 hours. The reaction mixture was diluted with EtOAc (50 mL), and washed sequentially with water (3×50 mL). The organic layer was dried over Na₂SO₄, filtered and evaporated to afford crude product. The crude product was purified by flash silica chromatography, elution gradient 0 to 100% EtOAc in petroleum ether. Pure fractions were evaporated to dryness to afford 9-benzyl-8-(4-(2-bromoethoxy)-2-chlorophenyl)-6-(1-methylcyclopropoxy)-9H-purine (3.00 g, 47%) as a white solid. ¹H NMR (300 MHz, CDCl₃): 0.87 (2H, t), 1.17 (2H, t), 1.82 (3H, s), 3.69 (2H, t), 4.36 (2H, t), 5.37 (2H, s), 6.84 (1H, dd), 6.9-6.99 (2H, m), 7.09 (1H, d), 7.14-7.26 (4H, m), 8.70 (1H, s). m/z: ES+[M+H]+ 515.

[R4] was made using a similar procedure described in Synthetic Example R3 for the synthesis of (S)-9-benzyl-8-(2-chloro-4-(2-(3-methylpiperazin-1-yl)ethoxy)phenyl)-6-(1-methylcyclopropoxy)-9H-purine, by N-alkylation reaction of 9-benzyl-8-(4-(2-bromoethoxy)-2-chlorophenyl)-6-(1-methylcyclopropoxy)-9H-purine (Synthetic Example R3, intermediate) with tert-butyl 3,8-diazabicyclo[3.2.1]octane-8-carboxylate and the BOC deprotection of the resultant tert-butyl (S)-4-(2-(4-(9-benzyl-6-(1-methylcyclopropoxy)-9H-purin-8-yl)-3-chlorophenoxy)ethyl)-2-methylpiperazine-1-carboxylate.

Tert-butyl (S)-4-(2-(4-(9-benzyl-6-(1-methylcyclopropoxy)-9H-purin-8-yl)-3-chlorophenoxy)ethyl)-2-methylpiperazine-1-carboxylate: ¹H NMR (300 MHz, DMSO-d₆): 0.82 (2H, t), 1.00 (2H, t), 1.37 (9H, s), 1.6-1.79 (7H, m), 2.24 (2H, d), 2.62-2.72 (4H, m), 3.94-4.09 (2H, m), 4.18 (2H, t), 5.27 (2H, s), 6.79-6.93 (2H, m), 7.02 (1H, dd), 7.14-7.22 (3H, m), 7.24 (1H, d), 7.40 (1H, d), 8.60 (1H, s). m/z: ES+[M+H]+ 645.

[R5] was made using a similar procedure described in Synthetic Example R3 for the synthesis of (S)-9-benzyl-8-(2-chloro-4-(2-(3-methylpiperazin-1-yl)ethoxy)phenyl)-6-(1-methylcyclopropoxy)-9H-purine, by N-alkylation of 9-benzyl-8-(4-(2-bromoethoxy)-2-chlorophenyl)-6-(1-methylcyclopropoxy)-9H-purine (Synthetic Example R3, intermediate) with tert-butyl 3,6-diazabicyclo[3.1.1]heptane-6-carboxylate and the BOC deprotection of the resultant tert-butyl (1R,5S)-3-(2-(4-(9-benzyl-6-(1-methylcyclopropoxy)-9H-purin-8-yl)-3-chlorophenoxy)ethyl)-3,6-diazabicyclo[3.1.1]heptane-6-carboxylate.

Tert-butyl (1R,5S)-3-(2-(4-(9-benzyl-6-(1-methylcyclopropoxy)-9H-purin-8-yl)-3-chlorophenoxy)ethyl)-3,6-diazabicyclo[3.1.1]heptane-6-carboxylate. ¹H NMR (300 MHz, DMSO-d₆): 0.84 (2H, t), 1.00 (2H, t), 1.33 (9H, s), 1.46-1.58 (1H, m), 1.72 (3H, s), 2.15-2.34 (1H, m), 2.75-2.87 (2H, m), 2.92 (2H, t), 3.01-3.25 (2H, m), 3.94 (2H, d), 4.19 (2H, t), 5.27 (2H, s), 6.8-6.95 (2H, m), 7.02 (1H, dd), 7.11-7.26 (4H, m), 7.41 (1H, d), 8.60 (1H, s). m/z: ES+[M+H]+ 631.

[R6] was made using a similar procedure described in Synthetic Example R3 for the synthesis of (S)-9-benzyl-8-(2-chloro-4-(2-(3-methylpiperazin-1-yl)ethoxy)phenyl)-6-(1-methylcyclopropoxy)-9H-purine, by N-alkylation of 9-benzyl-8-(4-(2-bromoethoxy)-2-chlorophenyl)-6-(1-methylcyclopropoxy)-9H-purine (Synthetic Example R3, intermediate) with tert-butyl (R)-2-methylpiperazine-1-carboxylate and the BOC deprotection of the resultant tert-butyl (R)-4-(2-(4-(9-benzyl-6-(1-methylcyclopropoxy)-9H-purin-8-yl)-3-chlorophenoxy)ethyl)-2-methylpiperazine-1-carboxylate.

Tert-butyl (R)-4-(2-(4-(9-benzyl-6-(1-methylcyclopropoxy)-9H-purin-8-yl)-3-chlorophenoxy)ethyl)-2-methylpiperazine-1-carboxylate. ¹H NMR (300 MHz, DMSO-d₆): 0.83 (2H, t), 1.00 (2H, t), 1.11 (3H, d), 1.38 (9H, s), 1.71 (3H, s), 1.92-2.04 (1H, m), 2.04-2.22 (1H, m), 2.59-2.8 (3H, m), 2.8-3.05 (2H, m), 3.54-3.72 (1H, m), 3.93-4.12 (1H, m), 4.19 (2H, t), 5.27 (2H, s), 6.83-6.96 (2H, m), 7.03 (1H, dd), 7.13-7.21 (3H, m), 7.25 (1H, d), 7.40 (1H, d), 8.60 (1H, s). m/z: ES+[M+H]+ 633.

[R7] was made by a similar procedure previously described in Synthetic Example R3 in the synthesis of tert-butyl (S)-4-(2-(4-(9-benzyl-6-(1-methylcyclopropoxy)-9H-purin-8-yl)-3-chlorophenoxy)ethyl)-2-methylpiperazine-1-carboxylate, by N-alkylation using 9-benzyl-8-(4-(2-bromoethoxy)-2-chlorophenyl)-6-(1-methylcyclopropoxy)-9H-purine (Synthetic Example R3, intermediate) and (R)-piperidin-3-ol as starting materials.

[R8] was made by a similar procedure previously described in Synthetic Example R3 in the synthesis of tert-butyl (S)-4-(2-(4-(9-benzyl-6-(1-methylcyclopropoxy)-9H-purin-8-yl)-3-chlorophenoxy)ethyl)-2-methylpiperazine-1-carboxylate, by N-alkylation using 9-benzyl-8-(4-(2-bromoethoxy)-2-chlorophenyl)-6-(1-methylcyclopropoxy)-9H-purine (Synthetic Example R3, intermediate) and 2,6-diazaspiro[3.4]octan-7-one as starting materials.

[R9] was made using a similar procedure described in Synthetic Example R3 in the synthesis of (S)-9-benzyl-8-(2-chloro-4-(2-(3-methylpiperazin-1-yl)ethoxy)phenyl)-6-(1-methylcyclopropoxy)-9H-purine, by N-alkylation of 9-benzyl-8-(4-(2-bromoethoxy)-2-chlorophenyl)-6-(1-methylcyclopropoxy)-9H-purine (Synthetic Example R3, intermediate) with tert-butyl (cis)-2,6-dimethylpiperazine-1-carboxylate and the BOC deprotection of the resultant tert-butyl (cis)-4-(2-(4-(9-benzyl-6-(1-methylcyclopropoxy)-9H-purin-8-yl)-3-chlorophenoxy)ethyl)-2,6-dimethylpiperazine-1-carboxylate.

Tert-butyl (cis)-4-(2-(4-(9-benzyl-6-(1-methylcyclopropoxy)-9H-purin-8-yl)-3-chlorophenoxy)ethyl)-2,6-dimethylpiperazine-1-carboxylate. ¹H NMR (300 MHz, DMSO-d₆): 0.85 (2H, t), 0.97-1.11 (8H, m), 1.39 (9H, s), 1.73 (3H, s), 2.50-2.62 (2H, m), 2.66-2.72 (2H, m), 3.00-3.08 (2H, m), 3.70-7.80 (2H, m), 4.11 (2H, t), 5.29 (2H, s), 6.90-6.93 (2H, m), 7.03 (1H, d), 7.16-7.28 (4H, m), 7.42 (1H, d), 8.62 (1H, s). m/z: ES+[M+H]+ 647.

[R10] was made using a similar procedure described in Synthetic Example R3 in the synthesis of (S)-9-benzyl-8-(2-chloro-4-(2-(3-methylpiperazin-1-yl)ethoxy)phenyl)-6-(1-methylcyclopropoxy)-9H-purine, by N-alkylation of 9-benzyl-8-(4-(2-bromoethoxy)-2-chlorophenyl)-6-(1-methylcyclopropoxy)-9H-purine (Synthetic Example R3, intermediate) with tert-butyl (S)-3-methylpiperazine-1-carboxylate and the BOC deprotection of the resultant tert-butyl (S)-4-(2-(4-(9-benzyl-6-(1-methylcyclopropoxy)-9H-purin-8-yl)-3-chlorophenoxy)ethyl)-3-methylpiperazine-1-carboxylate.

Tert-butyl (S)-4-(2-(4-(9-benzyl-6-(1-methylcyclopropoxy)-9H-purin-8-yl)-3-chlorophenoxy)ethyl)-3-methylpiperazine-1-carboxylate. ¹H NMR (300 MHz, DMSO-d₆): 0.86 (2H, t), 0.96-1.09 (5H, m), 1.40 (9H, s), 1.73 (3H, s), 2.21-2.47 (2H, m), 2.56-2.73 (2H, m), 2.78-2.87 (1H, m), 2.97-3.15 (2H, m), 3.49-3.7 (2H, m), 4.12-4.3 (2H, m), 5.29 (2H, s), 6.85-6.97 (2H, m), 6.99-7.08 (1H, m), 7.15-7.33 (4H, m), 7.42 (1H, d), 8.62 (1H, s). m/z: ES+[M+H]+ 633.

[R11] was made using a similar procedure described in Synthetic Example R3 in the synthesis of (S)-9-benzyl-8-(2-chloro-4-(2-(3-methylpiperazin-1-yl)ethoxy)phenyl)-6-(1-methylcyclopropoxy)-9H-purine, by N-alkylation of 9-benzyl-8-(4-(2-bromoethoxy)-2-chlorophenyl)-6-(1-methylcyclopropoxy)-9H-purine (Synthetic Example R3, intermediate) with tert-butyl (R)-3-methylpiperazine-1-carboxylate and the BOC deprotection of the resultant tert-butyl (R)-4-(2-(4-(9-benzyl-6-(1-methylcyclopropoxy)-9H-purin-8-yl)-3-chlorophenoxy)ethyl)-3-methylpiperazine-1-carboxylate.

Tert-butyl (R)-4-(2-(4-(9-benzyl-6-(1-methylcyclopropoxy)-9H-purin-8-yl)-3-chlorophenoxy)ethyl)-3-methylpiperazine-1-carboxylate. ¹H NMR (300 MHz, DMSO-d₆): 0.85 (2H, t), 0.96-1.08 (5H, m), 1.40 (9H, s), 1.74 (3H, s), 2.21-2.43 (2H, m), 2.64-2.85 (3H, m), 2.94-3.1 (2H, m), 3.5-3.64 (2H, m), 4.18 (2H, t), 5.29 (2H, s), 6.87-6.93 (2H, m), 7-7.06 (1H, m), 7.18-7.22 (3H, m), 7.26 (1H, d), 7.41 (1H, d), 8.62 (1H, s). m/z: ES+[M+H]+ 633.

[R12] The BOC deprotection of tert-butyl 4-(2-(4-(9-benzyl-6-(1-methylcyclopropoxy)-9H-purin-8-yl)-3-chlorophenoxy)ethyl)-3-oxo-1,4-diazepane-1-carboxylate followed a procedure previously described in Synthetic Example R3 in the synthesis of (S)-9-benzyl-8-(2-chloro-4-(2-(3-methylpiperazin-1-yl)ethoxy)phenyl)-6-(1-methylcyclopropoxy)-9H-purine.

Tert-butyl 4-(2-(4-(9-benzyl-6-(1-methylcyclopropoxy)-9H-purin-8-yl)-3-chlorophenoxy)ethyl)-3-oxo-1,4-diazepane-1-carboxylate was made by reaction of 9-benzyl-8-(4-(2-bromoethoxy)-2-chlorophenyl)-6-(1-methylcyclopropoxy)-9H-purine (Synthetic Example R3, intermediate) with tert-butyl 3-oxo-1,4-diazepane-1-carboxylate as follows:

Sodium hydride (8.56 mg, 0.21 mmol) was added to 9-benzyl-8-(4-(2-bromoethoxy)-2-chlorophenyl)-6-(1-methylcyclopropoxy)-9H-purine (100 mg, 0.19 mmol) and tert-butyl 3-oxo-1,4-diazepane-1-carboxylate (75 mg, 0.35 mmol) in DMF (3 mL) under nitrogen. The resulting mixture was stirred at 20° C. for 2 hours. The reaction mixture was concentrated and diluted with EtOAc (10 mL), and washed sequentially with water (3×15 mL). The organic layer was dried over Na₂SO₄, filtered and evaporated to afford crude product. The crude product was purified by flash silica chromatography, elution gradient 0 to 10% EtOH in DCM. Pure fractions were evaporated to dryness to afford tert-butyl 4-(2-(4-(9-benzyl-6-(1-methylcyclopropoxy)-9H-purin-8-yl)-3-chlorophenoxy)ethyl)-3-oxo-1,4-diazepane-1-carboxylate (70.0 mg, 56%) as a white solid. ¹H NMR (300 MHz, DMSO-d₆): 0.83 (2H, t), 0.99 (2H, t), 1.28 (9H, s), 1.7-1.78 (5H, m), 3.09-3.13 (2H, m), 3.39-3.44 (2H, m), 3.51-3.63 (2H, m), 3.68 (2H, t), 4.10 (2H, t), 5.27 (2H, s), 6.7-6.96 (2H, m), 7.00 (1H, dd), 7.14-7.22 (4H, m), 7.35-7.43 (1H, m), 8.60 (1H, s). m/z: ES+[M+H]+ 647.

[R13] Racemic 1-(2-(4-(9-benzyl-6-(1-methylcyclopropoxy)-9H-purin-8-yl)-3-chlorophenoxy)ethyl)-3-methylpiperazin-2-one was made using similar procedure described in Synthetic Example R12 in the synthesis of 1-(2-(4-(9-benzyl-6-(1-methylcyclopropoxy)-9H-purin-8-yl)-3-chlorophenoxy)ethyl)-1,4-diazepan-2-one, by N-alkylation of 9-benzyl-8-(4-(2-bromoethoxy)-2-chlorophenyl)-6-(1-methylcyclopropoxy)-9H-purine (Synthetic Example R12, intermediate) with tert-butyl (R)-2-methyl-3-oxopiperazine-1-carboxylate and the BOC deprotection of the resultant tert-butyl (R)-4-(2-(4-(9-benzyl-6-(1-methylcyclopropoxy)-9H-purin-8-yl)-3-chlorophenoxy)ethyl)-2-methyl-3-oxopiperazine-1-carboxylate.

Tert-butyl (R)-4-(2-(4-(9-benzyl-6-(1-methylcyclopropoxy)-9H-purin-8-yl)-3-chlorophenoxy)ethyl)-2-methyl-3-oxopiperazine-1-carboxylate. ¹H NMR (300 MHz, DMSO-d₆): 0.85 (2H, t), 1.01 (2H, t), 1.29 (3H, d), 1.41 (9H, s), 1.73 (3H, s), 3.19-3.23 (1H, m), 3.37-3.45 (1H, m), 3.5-3.65 (2H, m), 3.72-3.82 (1H, m), 3.87 (1H, d), 4.23 (2H, t), 4.27-4.35 (1H, m), 5.28 (2H, s), 6.88-6.92 (2H, m), 7.04 (1H, dd), 7.16-7.22 (3H, m), 7.26 (1H, d), 7.41 (1H, d), 8.61 (1H, s). m/z: ES+[M+H]+ 647.

The two enantiomers of racemic 1-(2-(4-(9-benzyl-6-(1-methylcyclopropoxy)-9H-purin-8-yl)-3-chlorophenoxy)ethyl)-3-methylpiperazin-2-one were purified by preparative chiral-HPLC on a CHIRALPAK IC-3 column, eluting isocratically with 25% DCM in hexane (modified with TEA) as eluent. The fractions containing the desired compound were evaporated to dryness to afford 1-(2-(4-(9-benzyl-6-(1-methylcyclopropoxy)-9H-purin-8-yl)-3-chlorophenoxy)ethyl)-3-methylpiperazin-2-one, isomer 2 (Synthetic Example R14, 30.0 mg, 27%) as a white solid and 1-(2-(4-(9-benzyl-6-(1-methylcyclopropoxy)-9H-purin-8-yl)-3-chlorophenoxy)ethyl)-3-methylpiperazin-2-one, isomer 1 (Synthetic Example R13 (30.0 mg, 27%).

[R15] was made using a similar procedure described in Synthetic Example R3 in the synthesis of (S)-9-benzyl-8-(2-chloro-4-(2-(3-methylpiperazin-1-yl)ethoxy)phenyl)-6-(1-methylcyclopropoxy)-9H-purine, by N-alkylation of 9-benzyl-8-(4-(2-bromoethoxy)-2-chlorophenyl)-6-(1-methylcyclopropoxy)-9H-purine (Synthetic Example R3, intermediate) with tert-butyl 3,6-diazabicyclo[3.1.1]heptane-3-carboxylate and the BOC deprotection of the resultant tert-butyl 6-(2-(4-(9-benzyl-6-(1-methylcyclopropoxy)-9H-purin-8-yl)-3-chlorophenoxy)ethyl)-3,6-diazabicyclo[3.1.1]heptane-3-carboxylate.

Tert-butyl 6-(2-(4-(9-benzyl-6-(1-methylcyclopropoxy)-9H-purin-8-yl)-3-chlorophenoxy)ethyl)-3,6-diazabicyclo[3.1.1]heptane-3-carboxylate. ¹H NMR (300 MHz, DMSO-d₆): 0.85 (2H, s), 1.03 (2H, s), 1.40 (1H, d), 1.44 (9H, s), 1.73 (3H, s), 2.35-2.46 (1H, m), 2.71 (2H, t), 3.27 (2H, s), 3.58 (4H, t), 4.10 (2H, t), 5.29 (2H, s), 6.91 (2H, dd), 7.01 (1H, dd), 7.21 (4H, dq), 7.41 (1H, d), 8.62 (1H, s). m/z: ES+[M+H]+ 631.

[R16] was made using a similar procedure described in Synthetic Example R12 in the synthesis of tert-butyl 4-(2-(4-(9-benzyl-6-(1-methylcyclopropoxy)-9H-purin-8-yl)-3-chlorophenoxy)ethyl)-3-oxo-1,4-diazepane-1-carboxylate (Synthetic Example R12, intermediate), by N-alkylation of 9-benzyl-8-(4-(2-bromoethoxy)-2-chlorophenyl)-6-(1-methylcyclopropoxy)-9H-purine (Synthetic Example R3, intermediate) with benzyl 2,2-dimethyl-3-oxopiperazine-1-carboxylate to afford benzyl 4-(2-(4-(9-benzyl-6-(1-methylcyclopropoxy)-9H-purin-8-yl)-3-chlorophenoxy)ethyl)-2,2-dimethyl-3-oxopiperazine-1-carboxylate. ¹H NMR (300 MHz, DMSO-d₆): 0.86 (2H, t), 1.01 (2H, t), 1.56 (6H, d), 1.73 (3H, s), 3.47-3.55 (2H, m), 3.62-3.69 (2H, m), 3.73 (2H, t), 4.23 (2H, t), 5.09 (2H, s), 5.28 (2H, s), 6.83-6.93 (2H, m), 7.04 (1H, dd), 7.14-7.23 (3H, m), 7.27 (1H, d), 7.34-7.43 (6H, m), 8.62 (1H, s). m/z: ES+[M+H]+ 695.

This step was followed by a Cbz deprotection of the resultant benzyl 4-(2-(4-(9-benzyl-6-(1-methylcyclopropoxy)-9H-purin-8-yl)-3-chlorophenoxy)ethyl)-2,2-dimethyl-3-oxopiperazine-1-carboxylate as follows:

Palladium on charcoal (10%) (18.37 mg, 0.17 mmol) was added to benzyl 4-(2-(4-(9-benzyl-6-(1-methylcyclopropoxy)-9H-purin-8-yl)-3-chlorophenoxy)ethyl)-2,2-dimethyl-3-oxopiperazine-1-carboxylate (120 mg, 0.17 mmol) in MeOH (3.0 mL) under hydrogen. The resulting mixture was stirred at 50° C. for 5 hours. The reaction mixture was filtered through filter paper. The solvent was removed under reduced pressure. The crude product was purified by preparative HPLC Column: XBridge Prep OBD C18 Column, 30*150 mm, 5 μm), using decreasingly polar mixtures of water (containing 10 mmol/L NH₄HCO_{3+0.1}% NH₃.H₂O) and MeCN as eluents. Fractions containing the desired compound were evaporated to dryness to afford 1-(2-(4-(9-benzyl-6-(1-methylcyclopropoxy)-9H-purin-8-yl)-3-chlorophenoxy)ethyl)-3,3-dimethylpiperazin-2-one (70 mg, 72%) as a white solid.

[R17] was made using a similar procedure described in Synthetic Example R3 in the synthesis of (S)-9-benzyl-8-(2-chloro-4-(2-(3-methylpiperazin-1-yl)ethoxy)phenyl)-6-(1-methylcyclopropoxy)-9H-purine, by N-alkylation of 9-benzyl-8-(4-(2-bromoethoxy)-2-chlorophenyl)-6-(1-methylcyclopropoxy)-9H-purine (Synthetic Example R3, intermediate) with tert-butyl 4,7-diazaspiro[2.5]octane-4-carboxylate and the BOC deprotection of the resultant tert-butyl 7-(2-(4-(9-benzyl-6-(1-methylcyclopropoxy)-9H-purin-8-yl)-3-chlorophenoxy)ethyl)-4,7-diazaspiro[2.5]octane-4-carboxylate.

Tert-butyl 7-(2-(4-(9-benzyl-6-(1-methylcyclopropoxy)-9H-purin-8-yl)-3-chlorophenoxy)ethyl)-4,7-diazaspiro[2.5]octane-4-carboxylate. ¹H NMR (300 MHz, DMSO-d₆): 0.70 (2H, t), 0.8-0.88 (4H, m), 1.03 (2H, t), 1.40 (9H, s), 1.73 (3H, s), 2.29-2.35 (2H, m), 2.40-2.50 (2H, m), 2.66-2.72 (2H, m), 2.90 (2H, s), 4.18 (2H, t), 5.29 (2H, s), 6.87-6.94 (2H, m), 7.03 (1H, dd), 7.17-7.24 (3H, m), 7.26 (1H, d), 7.41 (1H, d), 8.62 (1H, s). m/z: ES+[M+H]+ 645.

[R18] was made using a similar procedure described in Synthetic Example R3 in the synthesis of (S)-9-benzyl-8-(2-chloro-4-(2-(3-methylpiperazin-1-yl)ethoxy)phenyl)-6-(1-methylcyclopropoxy)-9H-purine, by N-alkylation of 9-benzyl-8-(4-(2-bromoethoxy)-2-chlorophenyl)-6-(1-methylcyclopropoxy)-9H-purine (Synthetic Example R3, intermediate) with tert-butyl (R)-2-(hydroxymethyl)piperazine-1-carboxylate and the BOC deprotection of the resultant tert-butyl (R)-4-(2-(4-(9-benzyl-6-(1-methylcyclopropoxy)-9H-purin-8-yl)-3-chlorophenoxy)ethyl)-2-(hydroxymethyl)piperazine-1-carboxylate.

Tert-butyl (R)-4-(2-(4-(9-benzyl-6-(1-methylcyclopropoxy)-9H-purin-8-yl)-3-chlorophenoxy)ethyl)-2-(hydroxymethyl)piperazine-1-carboxylate. ¹H NMR (300 MHz, DMSO-d₆): 0.86 (2H, t), 1.02 (2H, t), 1.40 (9H, s), 1.74 (3H, s), 1.93-2.14 (2H, m), 2.72 (2H, t), 2.81-2.96 (2H, m), 3.01-3.12 (1H, m), 3.36-3.42 (1H, m), 3.55-3.76 (2H, m), 3.90 (1H, s), 4.20 (2H, t), 4.69 (1H, t), 5.30 (2H, s), 6.83-6.95 (2H, m), 7.02-7.1 (1H, m), 7.15-7.31 (4H, m), 7.41 (1H, d), 8.62 (1H, s). m/z: ES+[M+H]+ 649.

[R19] was obtained using a similar method to (S)-9-benzyl-8-(2-chloro-4-(2-(3-methylpiperazin-1-yl)ethoxy)phenyl)-6-(1-methylcyclopropoxy)-9H-purine tert-butyl (1S,4S)-2,5-diazabicyclo[2.2.1]heptane-2-carboxylate (Synthetic Example R3), starting from 9-benzyl-8-(4-(2-bromoethoxy)-2-chlorophenyl)-6-(1-methylcyclopropoxy)-9H-purine and tert-butyl (1S,4S)-2,5-diazabicyclo[2.2.1]heptane-2-carboxylate.

[R20] was obtained using a similar method to (S)-9-benzyl-8-(2-chloro-4-(2-(3-methylpiperazin-1-yl)ethoxy)phenyl)-6-(1-methylcyclopropoxy)-9H-purine tert-butyl (1R,4R)-2,5-diazabicyclo[2.2.1]heptane-2-carboxylate (Synthetic Example R3), starting from 9-benzyl-8-(4-(2-bromoethoxy)-2-chlorophenyl)-6-(1-methylcyclopropoxy)-9H-purine and tert-butyl (1R,4R)-2,5-diazabicyclo[2.2.1]heptane-2-carboxylate.

[R21] was made using a similar procedure described in Synthetic Example R3 in the synthesis of (S)-9-benzyl-8-(2-chloro-4-(2-(3-methylpiperazin-1-yl)ethoxy)phenyl)-6-(1-methylcyclopropoxy)-9H-purine, by N-alkylation of 9-benzyl-8-(4-(2-bromoethoxy)-2-chlorophenyl)-6-(1-methylcyclopropoxy)-9H-purine (Synthetic Example R3, intermediate) with tert-butyl (1S,4S)-2,5-diazabicyclo[2.2.2]octane-2-carboxylate and the BOC deprotection of tert-butyl (1S,4S)-5-(2-(4-(9-benzyl-6-(1-methylcyclopropoxy)-9H-purin-8-yl)-3-chlorophenoxy)ethyl)-2,5-diazabicyclo[2.2.2]octane-2-carboxylate.

Tert-butyl (1S,4S)-5-(2-(4-(9-benzyl-6-(1-methylcyclopropoxy)-9H-purin-8-yl)-3-chlorophenoxy)ethyl)-2,5-diazabicyclo[2.2.2]octane-2-carboxylate. ¹H NMR (300 MHz, DMSO-d₆): 0.85 (2H, t), 1.02 (2H, t), 1.32-1.5 (10H, m), 1.63-1.81 (5H, m), 1.86-1.99 (1H, m), 2.82-3.07 (5H, m), 3.13-3.27 (1H, m), 3.44-3.67 (1H, m), 3.75-3.95 (1H, m), 4.13 (2H, t), 5.29 (2H, s), 6.7-6.98 (2H, m), 6.98-7.14 (1H, m), 7.17-7.28 (4H, m), 7.41 (1H, d), 8.62 (1H, s). m/z: ES+[M+H]+ 645.

[R22] was made using a similar procedure described in Synthetic Example R3 in the synthesis of (S)-9-benzyl-8-(2-chloro-4-(2-(3-methylpiperazin-1-yl)ethoxy)phenyl)-6-(1-methylcyclopropoxy)-9H-purine, by N-alkylation of 9-benzyl-8-(4-(2-bromoethoxy)-2-chlorophenyl)-6-(1-methylcyclopropoxy)-9H-purine (Synthetic Example R3, intermediate) with tert-butyl 4,7-diazaspiro[2.5]octane-7-carboxylate and the BOC deprotection of tert-butyl 4-(2-(4-(9-benzyl-6-(1-methylcyclopropoxy)-9H-purin-8-yl)-3-chlorophenoxy)ethyl)-4,7-diazaspiro[2.5]octane-7-carboxylate.

Tert-butyl 4-(2-(4-(9-benzyl-6-(1-methylcyclopropoxy)-9H-purin-8-yl)-3-chlorophenoxy)ethyl)-4,7-diazaspiro[2.5]octane-7-carboxylate. ¹H NMR (300 MHz, DMSO-d₆): 0.50 (2H, t), 0.53-0.63 (2H, t), 0.85 (2H, t), 0.97-1.1 (2H, t), 1.40 (9H, s), 1.73 (3H, s), 2.86-2.94 (2H, m), 3.01-3.14 (2H, m), 3.14-3.23 (2H, m), 3.35-3.42 (2H, m), 4.07 (2H, t), 5.29 (2H, s), 6.83-6.94 (2H, m), 7.01 (1H, dd), 7.12-7.27 (4H, m), 7.40 (1H, d), 8.62 (1H, s). m/z: ES+[M+H]+ 645.

[R23] was made using a similar procedure described in Synthetic Example R3 in the synthesis of (S)-9-benzyl-8-(2-chloro-4-(2-(3-methylpiperazin-1-yl)ethoxy)phenyl)-6-(1-methylcyclopropoxy)-9H-purine, by N-alkylation of 9-benzyl-8-(4-(2-bromoethoxy)-2-chlorophenyl)-6-(1-methylcyclopropoxy)-9H-purine (Synthetic Example R3, intermediate) with tert-butyl (S)-2-(cyanomethyl)piperazine-1-carboxylate and the BOC deprotection of tert-butyl (S)-4-(2-(4-(9-benzyl-6-(1-methylcyclopropoxy)-9H-purin-8-yl)-3-chlorophenoxy)ethyl)-2-(cyanomethyl)piperazine-1-carboxylate.

Tert-butyl (S)-4-(2-(4-(9-benzyl-6-(1-methylcyclopropoxy)-9H-purin-8-yl)-3-chlorophenoxy)ethyl)-2-(cyanomethyl)piperazine-1-carboxylate. ¹H NMR (300 MHz, DMSO-d₆): 0.86 (2H, t), 1.03 (2H, t), 1.42 (9H, s), 1.74 (3H, s), 2.15-2.36 (2H, m), 2.7-2.85 (4H, m), 2.83-3.01 (4H, m), 3.74-3.80 (1H, m), 4.20 (2H, t), 5.29 (2H, s), 6.88-6.93 (2H, m), 7.04 (1H, dd), 7.17-7.28 (4H, m), 7.42 (1H, d), 8.62 (1H, s). m/z: ES+[M+H]+ 658.

The following examples in Table S were synthesised as stated in the notes at the bottom of Table S.

TABLE S

| Ex | | | | LCMS |
|---|---|---|---|---|
| # | Structure | Name | ¹H NMR | [M + H]⁺ |
| S1 | 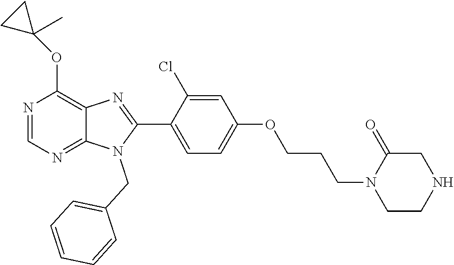 [SMILES: CC1(Oc2ncnc3c2nc(-c2ccc(OCCCN4CCNCC4=O)cc2Cl)n3Cc2ccccc2)CC1] | 1 -(3-(4-(9-benzyl- 6-(1- methylcyclopro- poxy)-9H-purin-8- yl)-3- chlorophenoxy)pro- pyl)piperazin-2- one | (400 MHz, DMSO-d₆): 0.80- 0.88 (2H, m), 1.01 (2H, d), 1.72 (3H, s), 1.96 (2H, p), 2.87 (2H, t), 3.20 (2H, s), 3.25 (2H, t), 3.43 (2H, t), 4.08 (2H, t), 5.29 (2H, s), 6.89 (2H, dd), 7.02 (1H, dd), 7.20 (4H, dd), 7.41 (1H, d), 8.61 (1H, s). One proton not observed. | 547 |
| S2 | 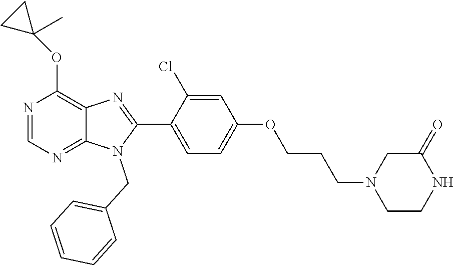 [SMILES: CC1(Oc2ncnc3c2nc(-c2ccc(OCCCN4CCNC(=O)C4)cc2Cl)n3Cc2ccccc2)CC1] | 4-(3-(4-(9-benzyl- 6-(1- methylcyclopropo- xy)-9H-purin-8- yl)-3- chlorophenoxy)pro- pyl)piperazin-2- one | (300 MHz, DMSO-d₆): 0.85 (2H, d), 1.01 (2H, d), 1.73 (3H, s), 1.96 (2H, p), 2.87 (2H, t), 3.20 (2H, s), 3.25 (2H, d), 3.43 (2H, s), 4.09 (2H, t), 5.29 (2H, s), 6.89 (2H, dd), 7.02 (1H, dd), 7.14- 7.29 (4H, m), 7.41 (1H, d), 8.61 (1H, s). One proton not observed. | 547 |
| S3 | 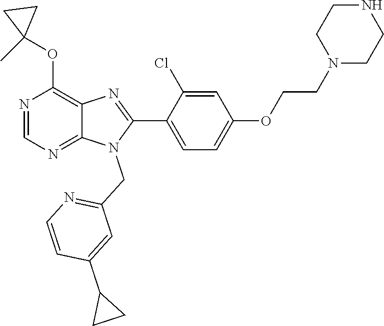 [SMILES: CC1(Oc2ncnc3c2nc(-c2ccc(OCCN4CCNCC4)cc2Cl)n3Cc2cc(C3CC3)ccn2)CC1] | 8-(2-chloro-4-(2- (piperazin-1- yl)ethoxy)phenyl)- 9-((4- cyclopropylpyridin- 2-yl)methyl)-6- (1- methylcyclopropo- xy)-9H-purine | (400 MHz, DMSO-d₆): 0.65 (2H, t), 0.85 (2H, t), 0.94-1.07 (4H, m), 1.73 (3H, s), 1.77-1.85 (1H, m), 2.34-2.39 (4H, m), 2.59-2.74 (6H, m), 4.14 (2H, t), 5.32 (2H, s), 6.74 (1H, s), 6.85-6.90 (1H, m), 6.92-6.98 (1H, m), 7.11-7.19 (1H, m), 7.28-7.34 (1H, m), 8.08-8.13 (1H, m), 8.56 (1H, s). One proton not observed. | 560 |
| S4 | 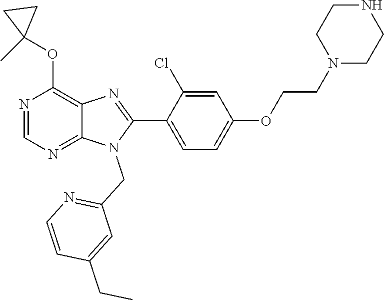 [SMILES: CCc1ccnc(Cn2c(-c3ccc(OCCN4CCNCC4)cc3Cl)nc3c(OC4(C)CC4)ncnc32)c1] | 8-(2-chloro-4-(2- (piperazin-1- yl)ethoxy)phenyl)- 9-((4-ethylpyridin- 2-yl)methyl)-6-(1- methylcyclopropo- xy)-9H-purine | (400 MHz, DMSO-d₆): 0.84 (2H, t), 1.00-1. 03 (2H, m), 1.07 (3H, t), 1.74 (3H, s), 2.33-2.43 (4H, m), 2.44-2.49 (2H, m), 2.65 (2H, t), 2.69 (4H, t), 4.13 (2H, t), 5.36 (2H, s), 6.87 (1H, d), 6.94 (1H, dd), 7.05 (1H, d), 7.19 (1H, d), 7.32 (1H, d), 8.19 (1H, d), 8.57 (1H, s). One proton not observed. | 548 |
| S5 | 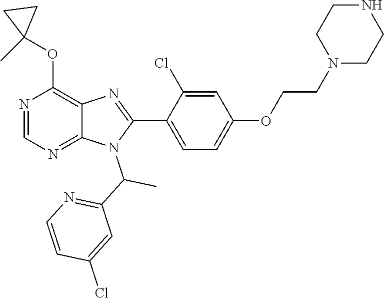 [SMILES: CC(c1cc(Cl)ccn1)n1c(-c2ccc(OCCN3CCNCC3)cc2Cl)nc2c(OC3(C)CC3)ncnc21] | 8-(2-chloro-4-(2- (piperazin-1- yl)ethoxy)phenyl)- 9-(1-(4- chloropyridin-2- yl)ethyl)-6-(1- methylcyclopropo- xy)-9H-purine (Isomer 2) | (400 MHz, DMSO-d₆): 0.84 (2H, t), 1.01 (2H, t), 1.71 (3H, s), 2.03 (3H, d), 2.36-2.43 (4H, m), 2.63-2.72 (6H, m), 4.17 (2H, t), 5.45-5.49 (1H, m), 7.04 (1H, dd), 7.26 (1H, d), 7.41-7.45 (3H, m), 8.38 (1H, d), 8.49 (1H, s). One proton not observed. | 568 |
| | Isomer 2 | | | |
| S6 | 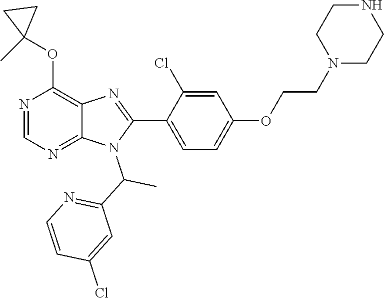 [SMILES: CC(c1cc(Cl)ccn1)n1c(-c2ccc(OCCN3CCNCC3)cc2Cl)nc2c(OC3(C)CC3)ncnc21] | 8-(2-chloro-4-(2- (piperazin-1- yl)ethoxy)phenyl)- 9-(1-(4- chloropyridin-2- yl)ethyl)-6-(1- methylcyclopropo- xy)-9H-purine (Isomer 1) | (400 MHz, DMSO-d₆): 0.84 (2H, t), 1.00 (2H, t), 1.71 (3H, s), 2.03 (3H, d), 2.36-2.43 (4H, m), 2.63-2.71 (6H, m), 4.17 (2H, t), 5.45-5.49 (1H, m), 7.04 (1H, dd), 7.26 (1H, d), 7.39-7.46 (3H, m), 8.38 (1H, d), 8.49 (1H, s). One proton not observed. | 568 |
| | Isomer 1 | | | |
| S7 | 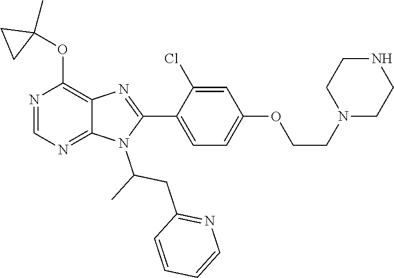 [SMILES: CC(Cc1ccccn1)n1c(-c2ccc(OCCN3CCNCC3)cc2Cl)nc2c(OC3(C)CC3)ncnc21] | 8-(2-chloro-4-(2- (piperazin-1- yl)ethoxy)phenyl)- 6-(1- methylcyclopropo- xy)-9-(1-(pyridin- 2-yl)propan-2-yl)- 9H-purine (Isomer 2) | (400 MHz, DMSO-d₆, 80° C.): 0.77 (2H, t), 0.97 (2H, t), 1.73-1.88 (6H, m), 2.45-2.61 (4H, m), 2.65-2.71 (6H, m), 3.27- 3.30 (1H, m), 3.68-3.71 (1H, m), 4.17 (2H, t), 4.48-4.6 (1H, m), 6.78- 6.81 (2H, m), 6.92 (1H, d), 7.08-7.19 (2H, m), 7.46-7.53 (1H, m), 8.28 (1H, d), 8.58 (1H, s). One proton not observed. | 548 |
| | Isomer 2 | | | |
| S8 | 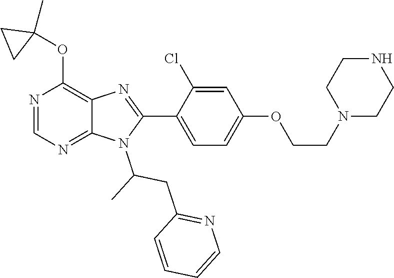 [SMILES: CC(Cc1ccccn1)n1c(-c2ccc(OCCN3CCNCC3)cc2Cl)nc2c(OC3(C)CC3)ncnc21] | 8-(2-chloro-4-(2- (piperazin-1- yl)ethoxy)phenyl)- 6-(1- methylcyclopropo- xy)-9-(1-(pyridin- 2-yl)propan-2-yl)- 9H-purine (Isomer 1) | (400 MHz, DMSO-d₆, 80° C.): 0.80 (2H, t), 0.98 (2H, t), 1.68-1.72 (6H, m), 2.38-2.42 (4H, m), 2.65-2.72 (6H, m), 3.26 (1H, dd), 3.68 (1H, dd), 4.17 (2H, t), 4.48- 4.59 (1H, m), 6.78 (2H, d), 6.92 (1H, dd), 7.09- 7.14 (1H, m), 7.16 (1H, d), 7.46-7.52 (1H, m), 8.28 (1H, d), 8.58 (1H, s). One proton not observed. | 548 |
| | Isomer 1 | | | |

[S1] was obtained by nucleophilic displacement of 9-benzyl-8-(4-(3-bromopropoxy)-2-chlorophenyl)-6-(1-methylcyclopropoxy)-9H-purine with tert-butyl 3-oxopiperazine-1-carboxylate, followed by BOC deprotection as follows:

Sodium hydride (16 mg, 0.40 mmol) was added to tert-butyl 3-oxopiperazine-1-carboxylate (120 mg, 0.60 mmol) and KI (6.6 mg, 0.04 mmol) in DMF (1 mL) at 0° C. The resulting mixture was stirred at rt for 15 minutes. Then 9-benzyl-8-(4-(3-bromopropoxy)-2-chlorophenyl)-6-(1-methylcyclopropoxy)-9H-purine (105 mg, 0.20 mmol) in DMF (1 mL) was added to the mixture. The resulting mixture was stirred at rt for 1 hour. The reaction mixture was quenched with saturated aq. NH₄Cl (15 mL) and extracted with EtOAc (3×20 mL). The organic layer was dried over Na₂SO₄, filtered and evaporated to afford crude tert-butyl 4-(3-(4-(9-benzyl-6-(1-methylcyclopropoxy)-9H-purin-8-yl)-3-chlorophenoxy)propyl)-3-oxopiperazine-1-carboxylate (0.2 g) as a yellow solid. The product was used in the next step directly without further purification.

HCl in dioxane (0.3 mL, 1.20 mmol, 4N) was added to tert-butyl 4-(3-(4-(9-benzyl-6-(1-methylcyclopropoxy)-9H-purin-8-yl)-3-chlorophenoxy)propyl)-3-oxopiperazine-1-carboxylate (180 mg, 0.28 mmol) in EtOAc (1 mL). The resulting mixture was stirred at rt for 2 hours. The solvent was removed under reduced pressure. The crude product was purified by preparative HPLC, Xselect CSH C18 OBD column, 30*150 mm, 5 μm, using decreasingly polar mixtures of water (0.1% formic acid) and MeCN as eluents. Fractions containing the desired compound were evaporated to dryness and purified again by preparative HPLC, YMC-Actus Triart C18 ExRS column, 30*150 mm, 5 μm, using decreasingly polar mixtures of water (containing 10 mmol/L NH₄HCO₃ and 0.1% aqueous ammonia) and MeCN as eluents. Fractions containing the desired compound were evaporated to dryness to afford 1-(3-(4-(9-benzyl-6-(1-methylcyclopropoxy)-9H-purin-8-yl)-3-chlorophenoxy)propyl)piperazin-2-one (13 mg, 8.5%) as a light yellow solid.

9-Benzyl-8-(4-(3-bromopropoxy)-2-chlorophenyl)-6-(1-methylcyclopropoxy)-9H-purine was synthesised as follows:

4-(9-Benzyl-6-(1-methylcyclopropoxy)-9H-purin-8-yl)-3-chlorophenol (300 mg, 0.74 mmol, Synthetic Example 5), 1,3-dibromopropane (223 mg, 1.11 mmol) and K₂CO₃ (306 mg, 2.21 mmol) in MeCN (6 mL) were stirred at 80° C. for 3 hours. The reaction mixture was diluted with water (25 mL) and extracted with EtOAc (3×25 mL). The organic layer was dried over Na₂SO₄, filtered and evaporated to afford a yellow gum. The residue was purified by preparative silica chromatography (eluting with pentane: EtOAc=1:1), to afford 9-benzyl-8-(4-(3-bromopropoxy)-2-chlorophenyl)-6-(1-methylcyclopropoxy)-9H-purine (0.17 g, 44%) as a yellow solid. ¹H NMR (300 MHz, DMSO-d₆): 0.84 (2H, t), 1.00 (2H, d), 1.72 (3H, s), 2.27 (2H, p), 3.67 (2H, t), 4.18 (2H, t), 5.28 (2H, s), 6.88 (2H, dt), 7.04 (1H, dt), 7.13-7.29 (4H, m), 7.41 (1H, dd), 8.61 (1H, s). m/z: ES+[M+H]+ 527.

[S2] was obtained by nucleophilic displacement of 9-benzyl-8-(4-(3-bromopropoxy)-2-chlorophenyl)-6-(1-methylcyclopropoxy)-9H-purine with tert-butyl 2-oxopiperazine-1-carboxylate and BOC deprotection, using a similar procedure as Synthetic Example S1, except that KI was omitted.

[S3] was obtained via BOC deprotection of tert-butyl 4-(2-(3-chloro-4-(9-((4-cyclopropylpyridin-2-yl)methyl)-6-(1-methylcyclopropoxy)-9H-purin-8-yl)phenoxy)ethyl)piperazine-1-carboxylate, using TFA.

The synthesis of tert-butyl 4-(2-(3-chloro-4-(9-((4-cyclopropylpyridin-2-yl)methyl)-6-(1-methylcyclopropoxy)-9H-purin-8-yl)phenoxy)ethyl)piperazine-1-carboxylate is described below:

A solution of cyclopropylzinc bromide (1M, 1.4 mL, 1.4 mmol) was added to a solution of tert-butyl 4-(2-(3-chloro-4-(9-((4-chloropyridin-2-yl)methyl)-6-(1-methylcyclopropoxy)-9H-purin-8-yl)phenoxy)ethyl)piperazine-1-carboxylate (300 mg, 0.46 mmol, Synthetic Example 10 intermediate) and tetrakis(triphenylphosphine)palladium (106 mg, 0.09 mmol) in THF (10 mL) at 0° C. over a period of 3 minutes under nitrogen. The resulting mixture was stirred at 60° C. for 16 hours. The reaction mixture was poured into saturated aq. NH₄Cl (50 mL) and extracted with EtOAc (3×50 mL). The organic layers were dried over Na₂SO₄, filtered and evaporated to afford a brown solid. The crude product was purified by flash silica chromatography, elution gradient 0 to 100% EtOAc in petroleum ether. Pure fractions were evaporated to dryness to afford tert-butyl 4-(2-(3-chloro-4-(9-((4-cyclopropylpyridin-2-yl)methyl)-6-(1-methylcyclopropoxy)-9H-purin-8-yl)phenoxy)ethyl)piperazine-1-carboxylate (210 mg, 69%) as a brown solid. ¹H NMR (400 MHz, DMSO-d₆): 0.66 (2H, t), 0.84 (2H, t), 0.95-1.05 (4H, m), 1.40 (9H, s), 1.74 (3H, s), 1.89-1.92 (1H, m), 2.38-2.46 (4H, m), 2.69-2.76 (2H, m), 3.33-3.45 (4H, m), 4.09-4.23 (2H, m), 5.26-5.42 (2H, m), 6.69-6.75 (1H, m), 6.84-6.91 (1H, m), 6.92-6.99 (1H, m), 7.24-7.33 (2H, m), 8.01-8.13 (1H, m), 8.57 (1H, s). m/z: ES+[M+H]+ 660

[S4] obtained via a similar procedure to 8-(2-chloro-4-(2-(piperazin-1-yl)ethoxy)phenyl)-9-((4-cyclopropylpyridin-2-yl)methyl)-6-(1-methylcyclopropoxy)-9H-purine [Synthetic Example S3], starting from tert-butyl 4-(2-(3-chloro-4-(9-((4-chloropyridin-2-yl)methyl)-6-(1-methylcyclopropoxy)-9H-purin-8-yl)phenoxy)ethyl)piperazine-1-carboxylate (300 mg, 0.46 mmol, Synthetic Example 10 intermediate) and diethylzinc, followed by Boc deprotection with TFA.

[S5] Racemic 8-(2-chloro-4-(2-(piperazin-1-yl)ethoxy)phenyl)-9-(1-(4-chloropyridin-2-yl)ethyl)-6-(1-methylcyclopropoxy)-9H-purine was obtained via a similar procedure to 8-(2-chloro-4-(2-(piperazin-1-yl)ethoxy)phenyl)-6-(1-methylcyclopropoxy)-9-(2-(pyridin-4-yl)ethyl)-9H-purine [Synthetic Example LI], starting from tert-butyl 4-(2-(3-chloro-4-(6-(1-methylcyclopropoxy)-9H-purin-8-yl)phenoxy)ethyl)piperazine-1-carboxylate and rac-1-(4-chloropyridin-2-yl)ethan-1-ol. Separation of enantiomers [Synthetic Example S5] and [Synthetic Example S6] was achieved by preparative chiral-HPLC on a CHIRALPAK IG-3, 4.6*50 mm, 3 μm column, eluting isocratically with 70% hexane in EtOH (modified with 0.1% DEA) as eluent.

[S7] Racemic 8-(2-chloro-4-(2-(piperazin-1-yl)ethoxy)phenyl)-6-(1-methylcyclopropoxy)-9-(1-(pyridin-2-yl)propan-2-yl)-9H-purine was obtained via a similar procedure to 9-benzyl-8-(2-chloro-4-(2-(4-methylpiperazin-1-yl)ethoxy)phenyl)-6-(1-methylcyclopropoxy)-9H-purine (Synthetic Example 4), using tert-butyl 4-(2-(3-chloro-4-formylphenoxy)ethyl)piperazine-1-carboxylate and 6-(1-methylcyclopropoxy)-N4-(1-(pyridin-2-yl)propan-2-yl)pyrimidine-4,5-diamine, followed by BOC deprotection with HCl. Separation of enantiomers [Synthetic Example S7] and [Synthetic Example S8] was achieved by preparative HPLC (Column: CHIRALPAK IA-3, 4.6*50 mm, 3 μm), eluting isocratically with 80% hexane in EtOH (modified with 0.1% DEA) as eluent.

6-(1-Methylcyclopropoxy)-N4-(1-(pyridin-2-yl)propan-2-yl)pyrimidine-4,5-diamine was made in a similar method as N4-benzyl-6-(1-methylcyclopropoxy)pyrimidine-4,5-diamine (Synthetic Example 4 intermediate), starting from 4,6-dichloro-5-nitropyrimidine and rac-1-(pyridin-2-yl)propan-2-amine. ¹H NMR (300 MHz, DMSO-d₆): 0.64-0.69 (2H, m), 0.83 (2H, t), 1.11 (3H, d), 1.59 (3H, s), 2.80 (1H, dd), 3.08 (1H, dd), 4.05 (2H, s), 4.47-4.59 (1H, m), 6.01 (1H, d), 7.16-7.22 (1H, m), 7.27 (1H, d), 7.64-7.74 (2H, m), 8.46-8.5 (1H, m). m/z: ES+[M+H]+ 300.

The compounds of Tables II and III were synthesized using methods similar to those describe herein.

All references cited herein, including patents, patent applications, papers, text books, and the like, and the references cited therein, to the extent that they are not already, are hereby incorporated herein by reference in their entirety for all purposes.

What is claimed:

1. A method of inserting a polynucleotide of interest into a genome of a eukaryotic cell, the method comprising:
a. adding an inhibitor of the microhomology-mediated end joining (MMEJ) pathway to a composition comprising the eukaryotic cell,
b. adding a Cas effector protein to the composition,
c. adding the polynucleotide of interest to the composition,
wherein the polynucleotide of interest is inserted into the genome by homology directed repair (HDR) or single-stranded template repair (SSTR),
wherein the inhibitor of the MMEJ pathway is an inhibitor of PolQ, selected from a compound of formula (I): 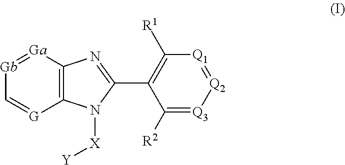
or any stereoisomer thereof or pharmaceutically acceptable salt thereof;
wherein,
R¹ and R² are each, independently, H, halo, C₁-C₃ alkyl, C₁-C₃ alkoxy, C₁-C₃ haloalkyl, C₁-C₃ hydroxyalkyl, —CN, C₂-C₄ alkyne, or C₂-C₆ alkoxyalkyl;
Q¹, Q², and Q³ are, independently N, C-L-R, or CR^{x}, wherein no more than one of Q¹, Q², and Q³ is C-L-R;
L is a bond, —O—; —C(O)—; —O(CH₂)ₚC(O)—; —C(O)NR^{y}—; —O(CH₂)ₚC(O)NR^{y}—; —O(CH₂)ₚNR^{y}; —NR—; —(CH₂)ₚ—; —(CH₂)ₚNR^{y}—; —(CH₂)ₚO—; —(CH₂)ₚC(O)—; —(CH₂)ₚC(₂)—; —O(CH₂)ₚ—;
p is, independently, 1, 2, or 3
R is H, R^{a}, R^{b}, R^{c}, or R^{d};
R^{a} is a 3-10 membered heterocycle optionally substituted with 1 to 4 substituents selected from amino, carboxy, halo, hydroxy, oxo, —CN, —S(O)₂OH, C1-C4 alkylamino, C1-C5 alkoxy, C2-C5 alkoxyalkyl, 4-6 membered heterocycle, and C1-C7 alkyl, wherein the C1-C7 alkyl is optionally substituted with 1 to 4 substituents selected from amino, carboxy, halo, hydroxy, oxo, —CN, C2-C8 ester, and C₁-C₅ alkoxy;
R^{b} is a C₁-C₇ alkyl, wherein one or two methylene groups from the C₁-C₇ alkyl are optionally replaced with NR^{e} or O and one or two single bonds in a C₂-C₇ alkyl chain are optionally replaced with a double or triple bond(s), wherein the C₁-C₇ alkyl is optionally substituted with 1 to 4 substituents selected from: halo, oxo, hydroxy, carboxyl, amino, —CN, C₂-C₄ alkynyl, C₂-C₆ carbamate, C₁-C₈ amide, C₁-C₄ sulfonyl, C₁-C₄ sulfonamide, C₁-C₄ alkylamino, C₁-C₅ alkoxy, C₃-C₆ carbocycle, and 3-10 membered heterocycle,
wherein the C₃-C₆ carbocycle is optionally substituted with 1 to 4 substituents selected from hydroxy, halo, and carboxy;
wherein the 3-10 membered heterocycle is optionally substituted with 1 to 4 substituents selected from amino, carboxy, halo, hydroxy, oxo, —CN, —S(O)₂₀H, C₁-C₄ alkylamino, C₁-C₅ alkoxy, C₂-C₅ alkoxyalkyl, 4-6 membered heterocycle, and C₁-C₇ alkyl, wherein the C₁-C₇ alkyl is optionally substituted with 1 to 4 substituents selected from amino, carboxy, halo, hydroxy, oxo, —CN, C₂-C₈ ester, and C₁-C₅ alkoxy;
R^{c} is a C₃-C₆ carbocycle optionally substituted with 1 to 4 substituents selected from hydroxy halo, and carboxy;
R^{d} is C₁-C₄ sulfonyl or C₁-C₄ sulfonamide;
R^{y} is H, C₁-C₃ alkyl, or C₁-3 haloalkyl;
R^{x} is H, halo, hydroxy, —CN, —NH₂, C₁-C₃ alkoxy, C₁-C₃ alkyl, or C₁₋₃ haloalkyl;
R^{e} is H, halo, C₁-C₈ alkyl, or C₁-C₈ haloalkyl;
X is a C₁-C₄ alkylene;
Y is a phenyl or 5-6 membered heteroaryl, wherein the phenyl or heteroaryl is optionally substituted with 1-3 substituents selected from halo, C₁-C₃ alkyl, C₁-C₃ alkoxy, —CN, C₁-C₃ haloalkyl, and cyclopropyl;
G is N or CH;
Gₐ and G_{b} are N, CH, or CR⁵ wherein one, and only one, of Gₐ and G_{b} is N or CH and one, and only one, of Gₐ and G_{b} is CR⁵;
R5 is 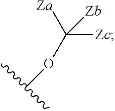
Zₐ and Z_{b} are, independently, C₁-C₃ alkyl or C₁-C₃ haloalkyl, or Zₐ and Z_{b} form a 3-6 membered carbocycle or heterocycle; and
Z_{c} is H, —CN, C₁-C₃ alkyl, C₁-C₃ haloalkyl, or C₂-C₄ alkyne, or combinations thereof.

2. The method of claim 1, wherein (a) further comprises adding an inhibitor of the non-homologous end joining (NHEJ) pathway.

3. The method of claims 1 or 2, further comprising: (d) adding a polynucleotide comprising:
an RNA guide sequence; a Cas-binding region; a DNA template sequence, or combinations thereof to the composition.

4. The method of any of claims 1-3, wherein the Cas effector protein is added in (b) by adding a Cas polynucleotide encoding the Cas effector protein.

5. The method of any of claims 1-4, wherein one or more of (i) the polynucleotide of interest, (ii) the polynucleotide of (d), or (iii) the Cas polynucleotide are encoded on a vector.

6. The method of any of claims 1-4, wherein (i) the polynucleotide of interest, (ii) the polynucleotide of step (d), and (iii) the Cas polynucleotide are encoded on a single vector.

7. The method of any of claims 1-6, wherein the polynucleotide of interest is added as DNA.

8. The method of any of claims 1-6, wherein the polynucleotide of step (d) is added as DNA.

9. The method any of claims 1-6, wherein the polynucleotide of step (d) is added as RNA.

10. The method of any of claims 1-6, wherein the Cas effector polynucleotide is added as DNA.

11. The method any of claims 1-6, wherein the Cas polynucleotide is added as RNA.

12. The method of any of claims 1-6, wherein the Cas polynucleotide is added as mRNA.

13. The method of claim 5 or 6, wherein the vector is a viral vector.

14. The method of claim 13, wherein the viral vector is a retrovirus, a lentivirus, an adenovirus, or an adeno-associated virus (AAV).

15. The method of claim 3, wherein the Cas effector protein and the polynucleotide of (d) are added in the form of a ribonucleoprotein (RNP).

16. The method of any of claims 1-15, wherein the Cas effector protein, the polynucleotide of interest, and the polynucleotide of (d) are added to the cell by microinjection, electroporation, or via a lipid nanoparticle, liposome, exosome, gold nanoparticle, or a DNA nanoclew.

17. The method of claims 5 or 9, wherein the vector is added to the composition by transfecting the eukaryotic cell.

18. The method of any of claims 1-17, wherein the Cas effector protein is a Cas9 nuclease, a Cas12a nuclease, or a Cas12f nuclease.

19. The method of claim 18, wherein the Cas effector protein is a Cas9 nuclease.

20. The method of claim 19, wherein the Cas9 nuclease is a Cas9 nuclease fused to a reverse transcriptase, a Cas9 nuclease fused to a DNA polymerase, a Cas9 nuclease fused to DN1S, a Cas9 nickase, a Cas9 fused to a Geminin degron domain, or a Cas9 nuclease fused to CTIP.

21. The method of any of claims 1-20, wherein the polynucleotide of interest is added via a vector.

22. The method of claim 21, wherein the vector is a viral vector.

23. The method of claim 22, wherein the viral vector is a retrovirus, a lentivirus, an adenovirus, or an adeno-associated virus (AAV).

24. The method of any of claims 1-23, wherein the polynucleotide of interest comprises a gene of interest.

25. The method of any of claims 1-23, wherein the polynucleotide of interest is 1 to 50 base pairs in length.

26. The method of any of claims 1-23, wherein the polynucleotide of interest is 50 to 5000 base pairs in length.

27. The method of any of claims 1-23, wherein the polynucleotide of interest is single stranded.

28. The method of any of claims 1-23, wherein the polynucleotide of interest is double stranded.

29. The method of any of claims 1-23, wherein the polynucleotide of interest is a hybrid polynucleotide comprising single-stranded and double-stranded regions.

30. The method of claim 29, wherein the hybrid polynucleotide comprises double-stranded sequences at the 5′ and 3′ ends and an internal single-stranded sequence.

31. The method of any of claims 1-28, wherein the polynucleotide of interest is double stranded with blunt ends.

32. The method of any of claims 1-30, wherein the polynucleotide of interest is double stranded with a 3′ overhang.

33. The method of any of claims 1-30, wherein the polynucleotide of interest is double stranded with a 5′ overhang.

34. The method of any of claims 1-29, wherein the polynucleotide of interest is a circular polynucleotide.

35. The method of any of claims 1-34, wherein the polynucleotide of interest comprises a chemical modification which enhances the stability, activity, distribution, or uptake of the polynucleotide.

36. The method of any of claims 1-35, wherein the inhibitor of Po1Q is a compound of formula (I), or any stereoisomer thereof or pharmaceutically acceptable salt thereof, wherein Q¹, Q², or Q³ is C-L-R^{b} and R^{b} is substituted with a 3-10 membered heterocycle, such as, but not limited to a N-heterocycle, such as, but not limited to a 4-7 membered N-heterocycle, wherein the heterocycle is substituted with C1-C7 alkyl, oxo, and/or halo, and combinations thereof.

37. The method of claim 36, wherein the inhibitor of Po1Q is a compound listed in Tables I, II, III, a pharmaceutically acceptable salt thereof, or combinations thereof.

38. The method of claim 1, wherein the inhibitor of Po1Q is 9-Benzyl-8-(2-chloro-4-(2-(4-methylpiperazin-1-yl)ethoxy)phenyl)-6-(1-methylcyclopropoxy)-9H-purine (Compound 1), or a pharmaceutically acceptable salt thereof.

39. The method of any of claims 1-38, wherein the concentration of the inhibitor of the MMEJ pathway in the composition is about 0.01 mM to about 1 mM.

40. The method of any of claims 1-38, wherein the concentration of the inhibitor of the MMEJ pathway in the composition is about 0.1 mM to about 100 mM.

41. The method of any of claims 2-38, wherein the inhibitor of the NHEJ pathway is an inhibitor of DNA-dependent protein kinase (DNA-PK).

42. The method of claim 41, wherein the inhibitor of DNA-PK is M3814, M9831/VX984, Nu7441, Nu7026, KU0060648, AZD7648, or combinations thereof.

43. The method of claim 42, wherein the inhibitor of DNA-PK is AZD7648.

44. The method of claim 41, wherein the inhibitor of DNA-PK is a peptide.

45. The method of any of claims 2-44, wherein the concentration of the inhibitor of the NHEJ pathway in the composition is about 0.01 M to about 1 mM.

46. The method of any of claims 2-44, wherein the concentration of the inhibitor of the NHEJ pathway in the composition is about 0.1 mM to about 100 mM.

47. The method of any of claims 1-46, wherein the inhibitor of the MMEJ pathway is added to the composition 0 minutes to about 48 hours before the Cas effector protein is added to the composition.

48. The method of any of claims 1-46, wherein the inhibitor of the MMEJ pathway is added to the composition 0 minutes to about 24 hours before the Cas effector protein is added to the composition.

49. The method of any of claims 1-46, wherein the inhibitor of the MMEJ pathway is added to the composition 0 minutes to about 6 hours before the Cas effector protein is added to the composition.

50. The method of any of claims 1-46, wherein the inhibitor of the MMEJ pathway is added to the composition 0 minutes to about 1 hour after the Cas effector protein is added to the composition.

51. The method of any of claims 2-50, wherein the inhibitor of the NHEJ pathway is added to the composition 0 minutes to about 48 hours before the Cas effector protein is added to the composition.

52. The method of any of claims 2-50, wherein the inhibitor of the NHEJ pathway is added to the composition 0 minutes to about 24 hours before the Cas effector protein is added to the composition.

53. The method of any of claims 2-50, wherein the inhibitor of the NHEJ pathway is added to the composition 0 minutes to about 6 hours before the Cas effector protein is added to the composition.

54. The method of any of claims 2-50, wherein the inhibitor of the NHEJ pathway is added to the composition 0 minutes to about 1 hour after the Cas effector protein is added to the composition.

55. The method of any of claims 2-54, wherein the inhibitor of the MMEJ pathway and the inhibitor of the NHEJ pathway are added to the composition at the same time.

56. The method of any of claims 2-54, wherein the inhibitor of the MMEJ pathway and the inhibitor of the NHEJ pathway are added to the composition at different times.

57. The method of any of claims 2-54, wherein the inhibitor of the MMEJ pathway, the inhibitor of the NHEJ pathway, and the Cas effector protein as added to the composition at the same time.

58. The method of any of claims 1-57, wherein the inhibitor of the MMEJ pathway is in the composition for about 1 to about 300 hours.

59. The method of any of claims 1-57, wherein the inhibitor of the MMEJ pathway is in the composition for about 10 to about 100 hours.

60. The method of any of claims 1-57, wherein the inhibitor of the MMEJ pathway is added at least once, at least twice, or at least three times.

61. The method of any of claims 2-60, wherein the inhibitor of the NHEJ pathway is in the composition for about 1 to about 300 hours.

62. The method of any of claims 2-60, wherein the inhibitor of the NHEJ pathway is in the composition for about 10 to about 100 hours.

63. The method of any of claims 2-60, wherein the inhibitor of the NHEJ pathway is added at least once, at least twice, or at least three times.

64. The method of any of claims 1-63 wherein the composition comprising the eukaryotic cells is a cell culture.

65. The method of claim 64, wherein the cell culture is an in vitro cell culture or an ex vivo cell culture.

66. The method of any of claims 1-65, wherein the eukaryotic cell is in vivo.

67. The method of claim 64, wherein the cell culture comprises a cell extract.

68. The method of any of claims 1-67, wherein the eukaryotic cell is a lymphocyte.

69. The method of claim 68, wherein the lymphocyte comprises a chimeric antigen receptor (CAR) or a T cell receptor (TCR).

70. The method of any of claims 1-67, wherein the eukaryotic cell is a pluripotent stem cell.

71. The method of claim 70, wherein the pluripotent stem cell is an induced pluripotent stem cell.

72. The method of claim 64, wherein the cell culture is a mammalian cell culture.

73. A method of inserting a polynucleotide of interest into a genome of a eukaryotic cell, the method comprising:
a. adding an inhibitor of the microhomology-mediated end joining (MMEJ) pathway to a composition comprising the eukaryotic cell,
b. adding the polynucleotide of interest to the composition,
wherein the genome comprises a genomically integrated Cas polynucleotide, and
wherein the polynucleotide of interest is inserted into the genome by homology directed repair (HDR) or single-stranded template repair (SSTR),
wherein the inhibitor of the MMEJ pathway is an inhibitor of PolQ, selected from a compound of formula (I): 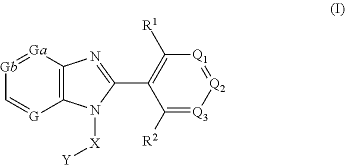
or any stereoisomer thereof or pharmaceutically acceptable salt thereof;
wherein,
R¹ and R² are each, independently, H, halo, C₁-C₃ alkyl, C₁-C₃ alkoxy, C₁-C₃ haloalkyl, C₁-C₃ hydroxyalkyl, —CN, C₂-C₄ alkyne, or C₂-C₆ alkoxyalkyl;
Q¹, Q², and Q³ are, independently N, C-L-R, or CR^{x}, wherein no more than one of Q¹, Q², and Q³ is C-L-R;
L is a bond, —O—; —C(O)—; —O(CH₂)ₚC(O)—; —C(O)NR^{y}—; —O(CH₂)ₚC(O)NR^{y}—; —O(CH₂)ₚNR^{y}; —NR^{y}—; —(CH₂)ₚ—; —(CH₂)ₚNR^{y}—; —(CH₂)ₚO—; —(CH₂)ₚC(O)—; —(CH₂)ₚC(O)O—; —O(CH₂)ₚ—;
p is, independently, 1, 2, or 3
R is H, R^{a}, R^{b}, R^{c}, or R^{d};
R^{a} is a 3-10 membered heterocycle optionally substituted with 1 to 4 substituents selected from amino, carboxy, halo, hydroxy, oxo, —CN, —S(O)₂OH, C₁-C₄ alkylamino, C₁-C₅ alkoxy, C₂-C₅ alkoxyalkyl, 4-6 membered heterocycle, and C₁-C₇ alkyl, wherein the C₁-C₇ alkyl is optionally substituted with 1 to 4 substituents selected from amino, carboxy, halo, hydroxy, oxo, —CN, C₂-C₈ ester, and C₁-C₅ alkoxy;
R^{b} is a C₁-C₇ alkyl, wherein one or two methylene groups from the C₁-C₇ alkyl are optionally replaced with NR^{e} or O and one or two single bonds in a C₂-C₇ alkyl chain are optionally replaced with a double or triple bond(s), wherein the C₁-C₇ alkyl is optionally substituted with 1 to 4 substituents selected from: halo, oxo, hydroxy, carboxyl, amino, —CN, C₂-C₄ alkynyl, C₂-C₆ carbamate, C₁-C₈ amide, C₁-C₄ sulfonyl, C₁-C₄ sulfonamide, C₁-C₄ alkylamino, C₁-C₅ alkoxy, C₃-C₆ carbocycle, and 3-10 membered heterocycle,
wherein the C₃-C₆ carbocycle is optionally substituted with 1 to 4 substituents selected from hydroxy, halo, and carboxy;
wherein the 3-10 membered heterocycle is optionally substituted with 1 to 4 substituents selected from amino, carboxy, halo, hydroxy, oxo, —CN, —S(O)₂₀H, C₁-C₄ alkylamino, C₁-C₅ alkoxy, C₂-C₅ alkoxyalkyl, 4-6 membered heterocycle, and C₁-C₇ alkyl, wherein the C₁-C₇ alkyl is optionally substituted with 1 to 4 substituents selected from amino, carboxy, halo, hydroxy, oxo, —CN, C₂-C₈ ester, and C₁-C₅ alkoxy;
R^{c} is a C₃-C₆ carbocycle optionally substituted with 1 to 4 substituents selected from hydroxy halo, and carboxy;
R^{d} is C₁-C₄ sulfonyl or C₁-C₄ sulfonamide;
R^{y} is H, C₁-C₃ alkyl, or C₁-3 haloalkyl;
R^{x} is H, halo, hydroxy, —CN, —NH₂, C₁-C₃ alkoxy, C₁-C₃ alkyl, or C₁-3 haloalkyl;
R^{e} is H, halo, C₁-C₈ alkyl, or C₁-C₈ haloalkyl;
X is a C₁-C₄ alkylene;
Y is a phenyl or 5-6 membered heteroaryl, wherein the phenyl or heteroaryl is optionally substituted with 1-3 substituents selected from halo, C₁-C₃ alkyl, C₁-C₃ alkoxy, —CN, C₁-C₃ haloalkyl, and cyclopropyl;
G is N or CH;
Gₐ and G_{b} are N, CH, or CR⁵ wherein one, and only one, of Gₐ and G_{b} is N or CH and one, and only one, of Gₐ and G_{b} is CR⁵;
R⁵ is 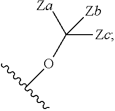
Zₐ and Z_{b} are, independently, C₁-C₃ alkyl or C₁-C₃ haloalkyl, or Zₐ and Z_{b} form a 3-6 membered carbocycle or heterocycle; and
Z_{c} is H, —CN, C₁-C₃ alkyl, C₁-C₃ haloalkyl, or C₂-C₄ alkyne, or combinations thereof.

74. The method of claim 73, wherein (a) further comprises adding an inhibitor of the non-homologous end joining (NHEJ) pathway to the composition.

75. The method of claims 73 or 74, further comprising: (c) adding a polynucleotide comprising: an RNA guide sequence; a Cas-binding region; a DNA template sequence, or combinations thereof to the composition.

76. The method of claim 75, wherein (i) the polynucleotide of interest and (ii) the polynucleotide of (c) are encoded on a vector.

77. The method of any of claims 73-76, wherein the polynucleotide of interest is added as DNA.

78. The method of any of claims 75-77, wherein the polynucleotide of (c) is added as DNA.

79. The method of any of claims 75-77, wherein the polynucleotide of (c) is added as RNA.

80. The method of claim 76, wherein the vector is a viral vector.

81. The method of claim 80, wherein the viral vector is a retrovirus, a lentivirus, an adenovirus, or an adeno-associated virus (AAV).

82. The method of claim 76, wherein the vector is added to the composition by transfecting the eukaryotic cell.

83. The method of any of claims 73-82, wherein the genomically integrated Cas polynucleotide is inducible.

84. The method of any of claims 73-83, wherein the Cas effector protein is a Cas9 nuclease, a Cas12a nuclease, or a Cas12f nuclease.

85. The method of claim 84, wherein the Cas effector protein is a Cas9 nuclease.

86. The method of claim 85, wherein the Cas9 nuclease is a Cas9 nuclease fused to a reverse transcriptase, a Cas9 nuclease fused to a DNA polymerase, a Cas9 nuclease fused to DN1S, a Cas9 nickase, a Cas9 fused to a Geminin degron domain or a Cas9 nuclease fused to CTIP.

87. The method of any of claims 73-86, wherein the polynucleotide of interest is added via a vector.

88. The method of claim 87, wherein the vector is a viral vector.

89. The method of claim 88, wherein the viral vector is a retrovirus, a lentivirus, an adenovirus, or an adeno-associated virus (AAV).

90. The method of any of claims 73-89, wherein the polynucleotide of interest comprises a gene of interest.

91. The method of any of claims 73-90, wherein the polynucleotide of interest is 1 to 50 base pairs in length.

92. The method of any of claims 73-90, wherein the polynucleotide of interest is 50 to 5000 base pairs in length.

93. The method of any of claims 73-90, wherein the polynucleotide of interest is single stranded.

94. The method of any of claims 73-90, wherein the polynucleotide of interest is double stranded.

95. The method of any of claims 73-90, wherein the polynucleotide of interest is a hybrid polynucleotide comprising single-stranded and double-stranded regions.

96. The method of claim 95, wherein the hybrid polynucleotide comprises double-stranded sequences at the 5′ and 3′ ends and an internal single-stranded sequence

97. The method of any of claims 73-90, wherein the polynucleotide of interest is double stranded with blunt ends.

98. The method of any of claims 73-90, wherein the polynucleotide of interest is double stranded with a 3′ overhang.

99. The method of any of claims 73-90, wherein the polynucleotide of interest is double stranded with a 5′ overhang.

100. The method of any of claims 73-90, wherein the polynucleotide is a circular polynucleotide.

101. The method of any of claims 73-100, wherein the polynucleotide comprises a chemical modification which enhances the stability, activity, distribution, or uptake of the polynucleotide.

102. The method of any of claims 73-101, wherein the inhibitor of Po1Q is a compound of formula (I), or any stereoisomer thereof or pharmaceutically acceptable salt thereof, wherein Q¹, Q², or Q³ is C-L-R^{b} and R^{b} is substituted with a 3-10 membered heterocycle, such as, but not limited to a N-heterocycle, such as, but not limited to a 4-7 membered N-heterocycle, wherein the heterocycle is substituted with C1-C7 alkyl, oxo, and/or halo, and combinations thereof.

103. The method of claim 102, wherein the inhibitor of Po1Q is a compound listed in Tables I, II, III, a pharmaceutically acceptable salt thereof, or combinations thereof.

104. The method of claim 102, wherein the inhibitor of Po1Q is 9-Benzyl-8-(2-chloro-4-(2-(4-methylpiperazin-1-yl)ethoxy)phenyl)-6-(1-methylcyclopropoxy)-9H-purine (Compound 1), or a salt thereof.

105. The method of any of claims 73-104, wherein the concentration of the inhibitor of the MMEJ pathway in the composition is about 0.01 mM to about 1 mM.

106. The method of any of claims 73-104, wherein the concentration of the inhibitor of the MMEJ pathway in the composition is about 0.1 mM to about 100 mM.

107. The method of any of claims 74-106, wherein the inhibitor of the NHEJ pathway is an inhibitor of DNA-dependent protein kinase (DNA-PK).

108. The method of claim 107, wherein the inhibitor of DNA-PK is M3814, M9831/VX984, Nu7441, Nu7026, KU0060648, AZD7648, or combinations thereof.

109. The method of claim 107, wherein the inhibitor of DNA-PK is a peptide.

110. The method of claim 109, wherein the inhibitor of DNA-PK is AZD7648.

111. The method of any of claims 74-110, wherein the concentration of the inhibitor of the NHEJ pathway in the composition is about 0.01 M to about 1 mM.

112. The method of any of claims 74-110, wherein the concentration of the inhibitor of the NHEJ pathway in the composition is about 0.1 mM to about 100 mM.

113. The method of any of claims 73-112, wherein the inhibitor of the MMEJ pathway is added to the composition 0 minutes to about 48 hours before induction of the genomically integrated Cas polynucleotide.

114. The method of any of claims 73-112, wherein the inhibitor of the MMEJ pathway is added to the composition 0 minutes to about 24 hours before induction of the genomically integrated Cas polynucleotide.

115. The method of any of claims 73-112, wherein the inhibitor of the MMEJ pathway is added to the composition 0 minutes to about 6 hours before induction of the genomically integrated Cas polynucleotide.

116. The method of any of claims 73-115, wherein the inhibitor of the NHEJ pathway is added to the composition 0 minutes to about 24 hours before the induction of the genomically integrated Cas polynucleotide.

117. The method of any of claims 74-115, wherein the inhibitor of the NHEJ pathway is added to the composition 0 minutes to about 24 hours before the induction of the genomically integrated Cas polynucleotide.

118. The method of any of claims 74-115, wherein the inhibitor of the NHEJ pathway is added to the composition 0 minutes to about 6 hours before induction of the genomically integrated Cas polynucleotide.

119. The method of any of claims 74-118, wherein the inhibitor of the MMEJ pathway and the inhibitor of the NHEJ pathway are added to the composition at the same time.

120. The method of any of claims 74-118, wherein the inhibitor of the MMEJ pathway and the inhibitor of the NHEJ pathway are added to the composition at different times.

121. The method of any of claims 74-120, wherein the inhibitor of the MMEJ pathway and the inhibitor of the NHEJ pathway are added to the composition at the same time as induction of the genomically integrated Cas polynucleotide.

122. The method of any of claims 73-121, wherein the inhibitor of the MMEJ pathway is in the composition for about 1 to about 300 hours.

123. The method of any of claims 73-121, wherein the inhibitor of the MMEJ pathway is in the composition for about 10 to about 100 hours.

124. The method of any of claims 73-123, wherein the inhibitor of the MMEJ pathway is added at least once, at least twice, or at least three times.

125. The method of any of claims 74-124, wherein the inhibitor of the NHEJ pathway is in the composition for about 1 to about 300 hours.

126. The method of any of claims 74-124, wherein the inhibitor of the NHEJ pathway is in the composition for about 10 to about 100 hours.

127. The method of any of claims 74-126, wherein the inhibitor of the NHEJ pathway is added at least once, at least twice, or at least three times.

128. The method of any of claims 73-127 wherein the composition comprising the eukaryotic cells is a cell culture.

129. The method of claim 128, wherein the cell culture is an in vitro cell culture or an ex vivo cell culture.

130. The method of any of claims 73-129, wherein the eukaryotic cell is in vivo.

131. The method of claim 130, wherein the cell culture comprises a cell extract.

132. The method of any of claims 73-131, wherein the eukaryotic cell is a lymphocyte.

133. The method of claim 132, wherein the lymphocyte comprises a chimeric antigen receptor or a T Cell receptor (TCR).

134. The method of any of claims 73-131, wherein the eukaryotic cell is a pluripotent stem cell.

135. The method of claim 134, wherein the pluripotent stem cell is an induced pluripotent stem cell.

136. The method of claim 131, wherein the cell culture is a mammalian cell culture.

137. A method of inserting a polynucleotide into a genome of a eukaryotic cell, the method comprising:
a. adding an inhibitor of the microhomology-mediated end joining (MMEJ) pathway to a composition comprising the eukaryotic cell,
b. transfecting the eukaryotic cell with:
i. a vector encoding a Cas effector protein,
ii. a vector comprising a polynucleotide of interest,
iii. a vector comprising a polynucleotide comprising: an RNA guide sequence; a Cas-binding region; a DNA template sequence, or combinations thereof,
wherein the vector of (i), (ii) and (iii) can be on the same vector or different vectors, and wherein the polynucleotide of interest is inserted into the genome by homology directed repair (HDR) or single-stranded template repair (SSTR),
wherein the inhibitor of the MMEJ pathway is an inhibitor of PolQ, selected from a compound of formula (I): 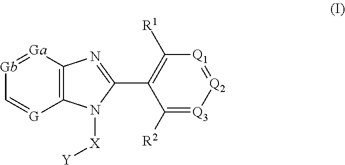
or any stereoisomer thereof or pharmaceutically acceptable salt thereof;
wherein,
R¹ and R² are each, independently, H, halo, C₁-C₃ alkyl, C₁-C₃ alkoxy, C₁-C₃ haloalkyl, C₁-C₃ hydroxyalkyl, —CN, C₂-C₄ alkyne, or C₂-C₆ alkoxyalkyl;
Q¹, Q², and Q³ are, independently N, C-L-R, or CR^{x}, wherein no more than one of Q¹, Q², and Q³ is C-L-R;
L is a bond, —O—; —C(O)—; —O(CH₂)ₚC(O)—; —C(O)NR^{y}—; —O(CH₂)ₚC(O)NR^{y}—; —O(CH₂)ₚNR^{y}; —NR^{y}—; —(CH₂)ₚ—; —(CH₂)ₚNR^{y}—; —(CH₂)ₚO—; —(CH₂)ₚC(O)—; —(CH₂)ₚC(O)O—; —O(CH₂)ₚ—;
p is, independently, 1, 2, or 3
R is H, R^{a}, R^{b}, R^{c}, or R^{d};
R^{a} is a 3-10 membered heterocycle optionally substituted with 1 to 4 substituents selected from amino, carboxy, halo, hydroxy, oxo, —CN, —S(O)₂OH, C₁-C₄ alkylamino, C₁-C₅ alkoxy, C₂-C₅ alkoxyalkyl, 4-6 membered heterocycle, and C₁-C₇ alkyl, wherein the C₁-C₇ alkyl is optionally substituted with 1 to 4 substituents selected from amino, carboxy, halo, hydroxy, oxo, —CN, C₂-C₈ ester, and C₁-C₅ alkoxy;
R^{b} is a C₁-C₇ alkyl, wherein one or two methylene groups from the C₁-C₇ alkyl are optionally replaced with NR^{e} or O and one or two single bonds in a C₂-C₇ alkyl chain are optionally replaced with a double or triple bond(s), wherein the C₁-C₇ alkyl is optionally substituted with 1 to 4 substituents selected from: halo, oxo, hydroxy, carboxyl, amino, —CN, C₂-C₄ alkynyl, C₂-C₆ carbamate, C₁-C₈ amide, C₁-C₄ sulfonyl, C₁-C₄ sulfonamide, C₁-C₄ alkylamino, C₁-C₅ alkoxy, C₃-C₆ carbocycle, and 3-10 membered heterocycle,
wherein the C₃-C₆ carbocycle is optionally substituted with 1 to 4 substituents selected from hydroxy, halo, and carboxy;
wherein the 3-10 membered heterocycle is optionally substituted with 1 to 4 substituents selected from amino, carboxy, halo, hydroxy, oxo, —CN, —S(O)₂₀H, C₁-C₄ alkylamino, C₁-C₅ alkoxy, C₂-C₅ alkoxyalkyl, 4-6 membered heterocycle, and C₁-C₇ alkyl, wherein the C₁-C₇ alkyl is optionally substituted with 1 to 4 substituents selected from amino, carboxy, halo, hydroxy, oxo, —CN, C₂-C₈ ester, and C₁-C₅ alkoxy;
R^{c} is a C₃-C₆ carbocycle optionally substituted with 1 to 4 substituents selected from hydroxy halo, and carboxy;
R^{d} is C₁-C₄ sulfonyl or C₁-C₄ sulfonamide;
R^{y} is H, C₁-C₃ alkyl, or C₁-3 haloalkyl;
R^{x} is H, halo, hydroxy, —CN, —NH₂, C₁-C₃ alkoxy, C₁-C₃ alkyl, or C₁-3 haloalkyl;
R^{e} is H, halo, C₁-C₈ alkyl, or C₁-C₈ haloalkyl;
X is a C₁-C₄ alkylene;
Y is a phenyl or 5-6 membered heteroaryl, wherein the phenyl or heteroaryl is optionally substituted with 1-3 substituents selected from halo, C₁-C₃ alkyl, C₁-C₃ alkoxy, —CN, C₁-C₃ haloalkyl, and cyclopropyl;
G is N or CH;
G^{a} and G^{b} are N, CH, or CR⁵ wherein one, and only one, of G^{a} and G^{b} is N or CH and one, and only one, of G^{a} and G^{b} is CR⁵;
R⁵ is 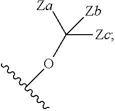
Zₐ and Z_{b} are, independently, C₁-C₃ alkyl or C₁-C₃ haloalkyl, or Zₐ and Z_{b} form a 3-6 membered carbocycle or heterocycle; and
Z_{c} is H, —CN, C₁-C₃ alkyl, C₁-C₃ haloalkyl, or C₂-C₄ alkyne, or combinations thereof.

138. The method of claim 137, further comprising adding an inhibitor of the non-homologous end joining (NHEJ) pathway to the composition comprising the eukaryotic cell.

139. The method of claims 137 or 138, wherein the Cas effector protein is encoded by a Cas polynucleotide.

140. The method of any of claims 137-139, wherein (i) the Cas effector protein and (ii) the polynucleotide of interest are encoded on a vector.

141. The method of any of claims 137-139, wherein the Cas effector protein and the polynucleotide of (iii) are encoded on a vector.

142. The method of any of claims 137-139, wherein the Cas effector protein, the polynucleotide of interest, and the polynucleotide of (iii) are encoded on a single vector.

143. The method of claims 137 or 138, wherein the Cas effector protein and the polynucleotide of (iii) are added in the form of a ribonucleoprotein (RNP).

144. A method of increasing the efficiency of homology directed repair (HDR) and single-stranded template repair (SSTR) gene insertions in a eukaryotic cell, the method comprising adding an inhibitor of the microhomology-mediated end joining (MMEJ) pathway when performing CRISPR/Cas mediated gene insertions in the eukaryotic cell, wherein the inhibitor of the MMEJ pathway is an inhibitor of PolQ, selected from a compound of formula (I), any stereoisomer thereof, pharmaceutically acceptable salt thereof, or combination thereof.

145. The method of claim 144, further comprising adding an inhibitor of the non-homologous end joining (NHEJ) pathway.

146. The method of claims 144 or 145, wherein the CRISPR/Cas-mediated gene insertion is a CRISPR/Cas9-mediated gene insertion.

147. A method of reducing microhomology-mediated end joining (MMEJ) pathway recombination during CRISPR/Cas mediated gene insertion in a cell, the method comprising adding an inhibitor of the microhomology-mediated end joining (MMEJ) pathway to the cell when performing Cas-mediated gene insertions, wherein the inhibitor of the MMEJ pathway is an inhibitor of PolQ, selected from a compound of formula (I), any stereoisomer thereof, pharmaceutically acceptable salt thereof, or combination thereof.

148. The method of claim 147, further comprising reducing non-homologous end joining (NHEJ) recombination during CRISPR/Cas-mediated gene insertion in a cell comprising adding an inhibitor of the non-homologous end joining (NHEJ) pathway to the cell.

149. The method of claims 147 or 148, wherein the CRISPR/Cas-mediated gene insertions are CRISPR/Cas9-mediated gene insertions.

150. A composition comprising:
a. a Cas effector protein or a vector encoding a Cas effector protein; and
b. an inhibitor of the microhomology-mediated end joining (MMEJ) pathway, wherein the inhibitor of the MMEJ pathway is an inhibitor of PolQ, selected from a compound of formula (I), any stereoisomer thereof, pharmaceutically acceptable salt thereof, or combination thereof.

151. The composition of claim 150, further comprising an inhibitor of the non-homologous end joining (NHEJ) pathway.

152. The composition of claims 150 or 151, further comprising a polynucleotide comprising: at least one RNA guide sequence; a Cas-binding region; a DNA template sequence, or combinations thereof.

153. The composition of any of claims 150-152, wherein the Cas effector protein is a Cas9 nuclease, a Cas12a nuclease, or a Cas12f nuclease.

154. The method of claim 153, wherein the Cas effector protein is a Cas9 nuclease.

155. The method of claim 154, wherein the Cas9 nuclease is a Cas9 nuclease fused to a reverse transcriptase, a Cas9 fused to a DNA polymerase, a Cas9 fused to DN1S, a Cas9 nickase, a Cas9 fused to a Geminin degron domain, or a Cas9 nuclease fused to CTIP.

156. The composition of any of claims 150-155, wherein the vector encoding a Cas effector protein is a viral vector.

157. The composition of any of claims 150-155, wherein the polynucleotide comprising at least one guide RNA sequence, a Cas-binding region, a DNA template sequence, or combinations thereof, is encoded on a vector.

158. The composition of claim 157, wherein the vector encoding the polynucleotide comprising at least one guide RNA sequence, a Cas-binding region, a DNA template sequence, or combinations thereof, is a viral vector.

159. The composition of claims 150 or 151, wherein the Cas effector protein and the polynucleotide comprising at least one guide RNA sequence, a Cas-binding region, a DNA template sequence, or combinations thereof, are in the form of a ribonucleoprotein (RNP).

160. The composition of any of claims 150-159, further comprising a pharmaceutically acceptable carrier, diluent, or excipient.

161. A kit comprising:
a. a Cas effector protein or a vector encoding a Cas effector protein; and
b. an inhibitor of the microhomology-mediated end joining (MMEJ) pathway, wherein the inhibitor of the MMEJ pathway is an inhibitor of PolQ, selected from a compound of formula (I), any stereoisomer thereof, pharmaceutically acceptable salt thereof, or combination thereof.

162. The kit of claim 161, further comprising an inhibitor of the non-homologous end joining (NHEJ) pathway.

163. The kit of claims 161 or 162, further comprising a polynucleotide comprising: at least one RNA guide sequence; a Cas-binding region; a DNA template sequence, or combinations thereof.

164. The kit of any of claims 161-163, wherein the Cas effector protein is a Cas9 nuclease, a Cas12a nuclease, or a Cas12f nuclease.

165. The kit of claim 164, wherein the Cas effector protein is a Cas9 nuclease.

166. The kit of claim 165, wherein the Cas9 nuclease is a Cas9 nuclease fused to a reverse transcriptase, a Cas9 fused to a DNA polymerase, a Cas9 fused to DN1S, a Cas9 nickase, a Cas9 fused to a Geminin degron domain, or a Cas9 nuclease fused to CTIP.

167. The kit of any of claims 161-166, wherein the vector encoding a Cas effector protein is a viral vector.

168. The kit of any of claims 161-167, wherein the guide polynucleotide is encoded on a vector.

169. The kit of claim 168, wherein the vector encoding the guide polynucleotide is a viral vector.

170. The kit of claims 161 or 162, wherein the Cas effector protein and the guide polynucleotide are in the form of a ribonucleoprotein (RNP).

171. The method, compound, or kit of any of claims 1-170 wherein Y is a phenyl or 5-6 membered heteroaryl, wherein the phenyl or heteroaryl is optionally substituted with 1-3 substituents selected from halo, C1-C3 alkyl, C1-C3 alkoxy, —CN, and C1-C3 haloalkyl.

172. The method, compound, or kit of any of claims 1-170 wherein the Po1Q wherein the inhibitor of Po1Q is a compound listed in Table I, a pharmaceutically acceptable salt thereof, or combinations thereof.

173. The method, compound, or kit of any of claims 1-170 wherein the Po1Q wherein the inhibitor of Po1Q is a compound listed in Tables II, III, a pharmaceutically acceptable salt thereof, or combinations thereof.
